(19) United States
(12) Patent Application Publication
(10) Pub. No.: US 2026/0176265 A1
(43) Pub. Date: Jun. 25, 2026

(54) PYRIDO[2,3-D]PYRIMIDIN-2-AMINE DERIVATIVES AS EGFR INHIBITORS FOR THE TREATMENT OF CANCER

(71) Applicant: Cancer Research Technology Limited, London (GB)

(72) Inventors: Michael J. Waring, Newcastle upon Tyne (GB); Hannah L. Stewart, Newcastle upon Tyne (GB); Matthew P. Martin, Newcastle upon Tyne (GB); Manon Sturbaut, Newcastle upon Tyne (GB); Steven Howard, Cambridge (GB); David Geoffrey Twigg, Cambridge (GB); John Walter Liebeschuetz, Cambridge (GB)

(21) Appl. No.: 19/123,183

(22) PCT Filed: Oct. 19, 2023
(86) PCT No.: PCT/GB2023/052719

§ 371 Date: Apr. 22, 2025

(30) Foreign Application Priority Data
Nov. 2, 2022 (GB) 2216305.9
Jan. 17, 2023 (GB) 2300686.9
Apr. 28, 2023 (GB) 2306377.9

(51) Int. Cl. C07D 471/04; A61K 31/519; A61K 31/5377; A61P 35/00

(52) U.S. Cl. CPC C07D 471/04; A61K 31/519; A61K 31/5377; A61P 35/00

(57) ABSTRACT

Provided are compounds of the Formula I, and salts, hydrates and solvates thereof:

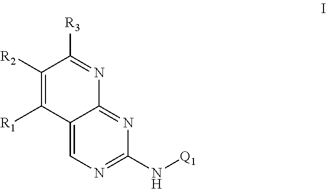

wherein R₁, R₂, R₃ and Q₁ are each as defined in the specification. The compounds are inhibitors of EGFR, including mutated forms of EGFR (for example EGFR comprising a T790M mutation, a deletion in exon 19 (e.g. A740-A750), an exon 20 insertion, a mutation at L858R and/or a C797S mutation) that confer resistance to existing EGFR inhibitors.

### INTRODUCTION

The present invention relates to novel therapeutic compounds. More specifically, the present invention relates to novel therapeutic compounds that are inhibitors of epidermal growth factor receptor (EGFR). The present invention also relates to pharmaceutical compositions comprising the novel therapeutic compounds defined herein, to processes for synthesising these compounds and to their use for the treatment of diseases and/or conditions in which EGFR activity is implicated including, but not limited to, the treatment and/or prevention of proliferative disorders (e.g. cancer).

### BACKGROUND OF THE INVENTION

EGFR (Epidermal Growth Factor Receptor) is a receptor tyrosine kinase in the erbB family (which also includes erbB2, erbB3 and erbB4). It controls a number of downstream signalling pathways in cells by binding a ligand, such as the epidermal growth factor (EGF). Binding of the ligand induces homodimerisation or heterodimerisation with other family members, which brings about autophosphorylation mediated by the EGFR kinase domain. This process triggers a signal transduction cascade.

EGFR signalling plays a key role in cellular proliferation, survival and suppression of apoptosis. These pathways can become disordered by overexpression or amplification of ligands or receptor, or through genetic alterations in EGFR. Aberrant EGFR signalling can be a key driver for oncogenic transformation, and tumour cell proliferation, invasion and metastasis. As an example, non-small cell lung cancer (NSCLC) patients frequently have activating mutations in EGFR, most commonly an in-frame deletion of Exon19, an L858R point mutation and missense mutations in Exon21 (Cancer Discovery, 2016, 6, 601).

Inhibitors of EGFR kinase activity, such as erlotinib, gefitinib, afatinib and osimertinib are effective treatments for EGFR mutated non-small cell lung cancer (Lancet Oncol. 2010, 11, 121; Lancet Oncol. 2016, 17, 577; J. Oncol. Pharm. Pract. 2020, 26, 1452; Lancet Oncol. 2011, 12, 735). Critical to the successful treatment is the selectivity of drugs for the mutated forms of EGFR relative to the wild-type since wild-type inhibition is associated with dose limiting side effects, such as skin rash and diarrhoea.

In almost all patients, treatment with first- and second-generation inhibitors, such as erlotinib, gefitinib and afatinib, leads to drug resistance after an average of 10-12 months (Lancet Oncol. 2010, 1, 121; Lancet Oncol. 2016 17, 577; Lancet Oncol. 2011, 12, 735). Most commonly, this resistance is due to a secondary mutation in the EGFR kinase domain T790M (J. Thorac. Oncol. 2009, 4, 1), which reduces the receptor's affinity for first- and second-generation drugs and increases its affinity for ATP (Proc. Natl. Acad. Sci. 2008, 105, 2070).

Third-generation EGFR inhibitors, such as osimertinib, have been developed to inhibit the T790M mutated forms of EGFR and have been shown to be active in both resistance mutant and activating mutant tumours and have become widely used in both first- and second-line treatment (N. Engl. J. Med. 2015, 372, 1689; N. Engl. J. Med. 2018, 378, 113). Osimertinib is a covalent inhibitor of EGFR that targets C797 (J. Med. Chem. 2014, 57, 8249). Resistance to third-generation therapies such as osimertinib develops with a duration of ca. 10 months. Commonly this resistance is attributable to mutations in the kinase domain that hinder the covalent binding, such as C797S (Brit. J. Cancer 2019, 121, 725).

There are no current treatments for cancers that are resistant to third-generation inhibitors. Hence, there is a high unmet need for inhibitors of activated forms of EGFR that harbour mutations, such as C797S that hinder the covalent binding of osimertinib, alongside other mutations such as Exon19Del and L858R, both with and without T790M, but with selectivity over the wild-type form.

The present invention was devised with the foregoing in mind.

### SUMMARY OF THE INVENTION

In one aspect, the present invention provides a compound of Formula I as defined herein, and/or a pharmaceutically acceptable salt, hydrate or solvate thereof.

In another aspect, the present invention provides a pharmaceutical composition which comprises a compound of Formula I as defined herein, or a pharmaceutically acceptable salt, hydrate or solvate thereof, and one or more pharmaceutically acceptable excipients.

In another aspect, the present invention provides a compound of Formula I as defined herein, or a pharmaceutically acceptable salt, hydrate or solvate thereof, or a pharmaceutical composition as defined herein, for use in therapy.

In another aspect, the present invention provides a compound of Formula I as defined herein, or a pharmaceutically acceptable salt, hydrate or solvate thereof, or a pharmaceutical composition as defined herein, for use in the treatment of a disease or condition in which EGFR activity is implicated.

In another aspect, the present invention provides a compound of Formula I as defined herein, or a pharmaceutically acceptable salt, hydrate or solvate thereof, or a pharmaceutical composition as defined herein, for use in the treatment of a disease or condition associated with aberrant activity of EGFR.

In another aspect, the present invention provides a compound of Formula I as defined herein, or a pharmaceutically acceptable salt, hydrate or solvate thereof, or a pharmaceutical composition as defined herein, for use in the treatment of proliferative disorders (e.g. cancer).

In another aspect, the present invention provides a compound of Formula I as defined herein, or a pharmaceutically acceptable salt, hydrate or solvate thereof, or a pharmaceutical composition as defined herein, for use in the treatment of cancer.

In another aspect, the present invention provides the use of a compound of Formula I as defined herein, or a pharmaceutically acceptable salt, hydrate or solvate thereof, in the manufacture of a medicament for use in the treatment of a disease or condition in which EGFR activity is implicated.

In another aspect, the present invention provides the use of a compound of Formula I as defined herein, or a pharmaceutically acceptable salt, hydrate or solvate thereof, in the manufacture of a medicament for use in the treatment of a disease or condition associated with aberrant activity of EGFR.

In another aspect, the present invention provides the use of a compound of Formula I as defined herein, or a pharmaceutically acceptable salt, hydrate or solvate thereof, in the manufacture of a medicament for use in the treatment of proliferative disorders (e.g. cancer or benign neoplasms).

In another aspect, the present invention the use of a compound of Formula I as defined herein, or a pharmaceutically acceptable salt, hydrate or solvate thereof, in the manufacture of a medicament for use in the treatment of a cancer.

In another aspect, the present invention provides a method of treating a disease or condition in which EGFR activity is implicated, said method comprising administering to a subject in need thereof an effective amount of a compound of Formula I as defined herein, or a pharmaceutically acceptable salt, hydrate or solvate thereof, or a pharmaceutical composition as defined herein.

In another aspect, the present invention provides a method of treating a disease or condition associated with aberrant activity of EGFR, said method comprising administering to a subject in need thereof an effective amount of a compound of Formula I as defined herein, or a pharmaceutically acceptable salt, hydrate or solvate thereof, or a pharmaceutical composition as defined herein.

In another aspect, the present invention provides a method of treating a proliferative disorder (e.g. cancer or benign neoplasms), said method comprising administering to a subject in need thereof an effective amount of a compound of Formula I as defined herein, or a pharmaceutically acceptable salt, hydrate or solvate thereof, or a pharmaceutical composition as defined herein.

In another aspect, the present invention provides a method of treating cancer, said method comprising administering to a subject in need thereof an effective amount of a compound of Formula I as defined herein, or a pharmaceutically acceptable salt, hydrate or solvate thereof, or a pharmaceutical composition as defined herein.

In another aspect, the present invention provides a compound of formula I, or a pharmaceutically acceptable salt, hydrate or solvate thereof, for use in the treatment of an EGFR positive cancer, optionally selected from head and neck cancer, brain cancer, breast cancer, colon cancer and/or lung cancer.

In another aspect, the present invention provides a compound of formula I, or a pharmaceutically acceptable salt, hydrate or solvate thereof, for use in the treatment of EGFR positive non-small cell lung cancer.

In another aspect, the present invention provides a compound of formula I, or a pharmaceutically acceptable salt, hydrate or solvate thereof, for use in the treatment of a cancer expressing a mutated form of EGFR (for example EGFR comprising a T790M mutation, a deletion in exon 19 (such as A740-A750), an exon 20 insertion, a mutation at L858R and/or a C797S mutation).

In another aspect, the present invention provides a compound of formula I or a pharmaceutically acceptable salt, hydrate or solvate thereof, for use in the treatment of non-small cell lung cancer expressing a mutated form of EGFR (for example EGFR comprising a T790M mutation, a deletion in exon 19 (such as A740-A750), an exon 20 insertion, a mutation at L858R and/or a C797S mutation).

In another aspect, the present invention provides a compound of formula I, or a pharmaceutically acceptable salt, hydrate or solvate thereof, for use in the treatment of a cancer resistant to treatment with a third generation EFGR inhibitor, e.g. osimertinib, lazertinib (YH25448), EGF816, olmutinib, PF-06747775, avitinib and/or rociletinib.

In another aspect, the present invention provides a compound of formula I, or a pharmaceutically acceptable salt, hydrate or solvate thereof, for use in the treatment of non-small cell lung cancer resistant to treatment with osimertinib.

In another aspect, the present invention provides the use of a compound of formula I, or a pharmaceutically acceptable salt, hydrate or solvate thereof, in the manufacture of a medicament for use in the treatment of an EGFR positive cancer, optionally selected from head and neck cancer, brain cancer, breast cancer, colon cancer and/or lung cancer.

In another aspect, the present invention provides the use of a compound of formula I, or a pharmaceutically acceptable salt, hydrate or solvate thereof, in the manufacture of a medicament for use in the treatment of EGFR positive non-small cell lung cancer.

In another aspect, the present invention provides the use of a compound of formula I, or a pharmaceutically acceptable salt, hydrate or solvate thereof, in the manufacture of a medicament for use in the treatment of a cancer expressing a mutated form of EGFR (for example EGFR comprising a T790M mutation, a deletion in exon 19 (such as A740-A750), an exon 20 insertion, a mutation at L858R and/or a C797S mutation).

In another aspect, the present invention provides the use of a compound of formula I or a pharmaceutically acceptable salt, hydrate or solvate thereof, in the manufacture of a medicament for use in the treatment of non-small cell lung cancer expressing a mutated form of EGFR (for example EGFR comprising a T790M mutation, a deletion in exon 19 (such as A740-A750), an exon 20 insertion, a mutation at L858R and/or a C797S mutation).

In another aspect, the present invention provides the use of a compound of formula I, or a pharmaceutically acceptable salt, hydrate or solvate thereof, in the manufacture of a medicament for use in the treatment of a cancer resistant to treatment with a third generation EFGR inhibitor, e.g. osimertinib, lazertinib (YH25448), EGF816, olmutinib, PF-06747775, avitinib and/or rociletinib.

In another aspect, the present invention provides the use of a compound of formula I, or a pharmaceutically acceptable salt, hydrate or solvate thereof, in the manufacture of a medicament for use in the treatment of non-small cell lung cancer resistant to treatment with osimertinib.

In another aspect, the present invention provides a method of treating an EGFR positive cancer, optionally selected from head and neck cancer, brain cancer, breast cancer, colon cancer and/or lung cancer, said method comprising administering to a subject in need thereof an effective amount of a compound of Formula I as defined herein, or a pharmaceutically acceptable salt, hydrate or solvate thereof, or a pharmaceutical composition as defined herein.

In another aspect, the present invention provides a method of treating EGFR positive non-small cell lung cancer, said method comprising administering to a subject in need thereof an effective amount of a compound of Formula I as defined herein, or a pharmaceutically acceptable salt, hydrate or solvate thereof, or a pharmaceutical composition as defined herein.

In another aspect, the present invention provides a method of treating a cancer expressing a mutated form of EGFR (for example EGFR comprising a T790M mutation, a deletion in exon 19 (such as A740-A750), an exon 20 insertion, a mutation at L858R and/or a C797S mutation), said method comprising administering to a subject in need thereof an effective amount of a compound of Formula I as defined herein, or a pharmaceutically acceptable salt, hydrate or solvate thereof, or a pharmaceutical composition as defined herein.

In another aspect, the present invention provides a method of treating non-small cell lung cancer expressing a mutated form of EGFR (for example EGFR comprising a T790M mutation, a deletion in exon 19 (such as A740-A750), an exon 20 insertion, a mutation at L858R and/or a C797S mutation), said method comprising administering to a subject in need thereof an effective amount of a compound of Formula I as defined herein, or a pharmaceutically acceptable salt, hydrate or solvate thereof, or a pharmaceutical composition as defined herein.

In another aspect, the present invention provides a method of treating a cancer resistant to treatment with a third generation EFGR inhibitor, e.g. osimertinib, lazertinib (YH25448), EGF816, olmutinib, PF-06747775, avitinib and/or rociletinib, said method comprising administering to a subject in need thereof an effective amount of a compound of Formula I as defined herein, or a pharmaceutically acceptable salt, hydrate or solvate thereof, or a pharmaceutical composition as defined herein.

In another aspect, the present invention provides a method of treating non-small cell lung cancer resistant to treatment with osimertinib, said method comprising administering to a subject in need thereof an effective amount of a compound of Formula I as defined herein, or a pharmaceutically acceptable salt, hydrate or solvate thereof, or a pharmaceutical composition as defined herein.

In another aspect, the present invention provides a combination treatment comprising a compound of Formula I, or a pharmaceutically acceptable salt, hydrate or solvate thereof, as defined herein, with one or more additional therapeutic agents.

In another aspect, the present invention provides processes for preparing compounds of Formula I as defined herein, or a pharmaceutically acceptable salt, hydrate or solvate thereof, as defined herein, with one or more additional therapeutic agents.

Preferred, suitable, and optional features of any one particular aspect of the present invention are also preferred, suitable, and optional features of any other aspect.

### DETAILED DESCRIPTION OF THE INVENTION

### Definitions

Unless otherwise stated, the following terms used in the specification and claims have the following meanings set out below.

It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

The compounds and intermediates described herein may be named according to either the IUPAC (International Union for Pure and Applied Chemistry) or CAS (Chemical Abstracts Service) nomenclature systems. It should be understood that unless expressly stated to the contrary, the terms “compounds of Formula I”, “compounds of the invention” and the more general term “compounds” refer to and include any and all compounds described by and/or with reference to Formula I herein. It should also be understood that these terms encompasses all stereoisomers, i.e. cis and trans isomers, as well as optical isomers, i.e. R and S enantiomers, of such compounds, in substantially pure form and/or any mixtures of the foregoing in any ratio. This understanding extends to pharmaceutical compositions and methods of treatment that employ or comprise one or more compounds of the Formula I, either by themselves or in combination with additional agents.

Unless specified otherwise, atoms are referred to herein by their chemical symbol as appearing in the IUPAC periodic table of the Elements. For example, “C” refers to a carbon atom.

The term “(m-nC)” or “(m-nC) group” used alone or as a prefix, refers to any group having m to n carbon atoms.

In this specification the term “alkyl” includes both straight and branched chain alkyl groups. References to individual alkyl groups such as “propyl” are specific for the straight chain version only and references to individual branched chain alkyl groups such as “isopropyl” are specific for the branched chain version only. For Example, “(1-6C)alkyl” includes (1-4C)alkyl, (1-3C)alkyl, propyl, isopropyl and t-butyl. A similar convention applies to other radicals, for example “phenyl(1-6C)alkyl” includes phenyl(1-4C)alkyl, benzyl, 1-phenylethyl and 2-phenylethyl.

An “alkylene” group is an alkyl group that is positioned between and serves to connect two other chemical groups. Thus, “(1-6C)alkylene” means a linear saturated divalent hydrocarbon radical of one to six carbon atoms or a branched saturated divalent hydrocarbon radical of three to six carbon atoms, for example, methylene, ethylene, propylene, 2-methylpropylene, pentylene, and the like.

“(3-6C)cycloalkyl” means a hydrocarbon ring containing from 3 to 6 carbon atoms, for example, cyclopropyl, cyclobutyl, cyclopentyl, cyclohexyl, cycloheptyl or bicyclo[2.2.1]heptyl.

The term “halo” or “halogeno” refers to fluoro, chloro, bromo and iodo.

As used herein by themselves or in conjunction with another term or terms, “haloalkyl” and “haloalkyl group” refer to alkyl groups in which one or more hydrogen atoms are replaced by halogen atoms. Representative examples include, but are not limited to, —CF₃, —CHF₂, —CH₂F, —CF₂CF₃, —CHFCF₃, and —CH₂CF_{3c} Suitably, a haloalkyl group is selected from —CHF₂ and —CF₃, suitably —CF₃.

As used herein by themselves or in conjunction with another term or terms, “haloalkoxy” and “haloalkoxy group” refer to alkoxy groups (i.e. O-alkyl groups) in which one or more hydrogen atoms are replaced by halogen atoms. Representative examples include, but are not limited to, —OCF₃, —OCHF₂, —OCH₂F, and —OCF₂CF_{3c} Suitably, a haloalkyoxy group is selected from —OCHF₂ and —OCF₃, suitably —OCF₃.

The term “heterocyclyl”, “heterocyclic” or “heterocycle” means a non-aromatic saturated or partially saturated monocyclic, fused, bridged, or spiro bicyclic heterocyclic ring system(s). Monocyclic heterocyclic rings contain from about 3 to 12 (suitably from 3 to 7) ring atoms, with from 1 to 5 (suitably 1, 2 or 3) heteroatoms selected from nitrogen, oxygen or sulfur in the ring. Bicyclic heterocycles contain from 7 to 17 member atoms, suitably 7 to 12 member atoms, in the ring. Bicyclic heterocyclic(s) rings may be fused, spiro, or bridged ring systems. Examples of heterocyclic groups include cyclic ethers such as, but not limited to, oxiranyl, oxetanyl, tetrahydrofuranyl, dioxanyl, and substituted cyclic ethers. Heterocycles containing nitrogen include, for example, azetidinyl, pyrrolidinyl, piperidinyl, piperazinyl, tetrahydrotriazinyl, tetrahydropyrazolyl, and the like. Typical sulfur containing heterocycles include tetrahydrothienyl, dihydro-1,3-dithiol, tetrahydro-2H-thiopyran, and hexahydrothiepine. Other heterocycles include dihydrooxathiolyl, tetrahydrooxazolyl, tetrahydro-oxadiazolyl, tetrahydrod ioxazolyl, tetrahydrooxathiazolyl, hexahydrotriazinyl, tetrahydrooxazinyl, morpholinyl, thiomorpholinyl, tetrahydropyrimidinyl, dioxolinyl, octahydrobenzofuranyl, octahydrobenzimidazolyl, and octahydrobenzothiazolyl. For heterocycles containing sulfur, the oxidized sulfur heterocycles containing SO or SO₂ groups are also included. Examples include the sulfoxide and sulfone forms of tetrahydrothienyl and thiomorpholinyl such as, but not limited to, tetrahydrothiene 1,1-dioxide and thiomorpholinyl 1,1-dioxide. A suitable value for a heterocyclyl group which bears 1 or 2 oxo (=O) or thioxo (=S) substituents is, for example, 2-oxopyrrolidinyl, 2-thioxopyrrolidinyl, 2-oxoimidazolidinyl, 2-thioxoimidazolidinyl, 2-oxopiperidinyl, 2,5-dioxopyrrolidinyl, 2,5-dioxoimidazolidinyl or 2,6-dioxopiperidinyl. Particular heterocyclyl groups are saturated monocyclic 3 to 7 membered heterocyclyls containing 1, 2 or 3 heteroatoms selected from nitrogen, oxygen or sulfur, for example azetidinyl, tetrahydrofuranyl, tetrahydropyranyl, pyrrolidinyl, morpholinyl, tetrahydrothienyl, tetrahydrothienyl 1,1-dioxide, thiomorpholinyl, thiomorpholinyl 1,1-dioxide, piperidinyl, homopiperidinyl, piperazinyl or homopiperazinyl. As the skilled person would appreciate, any heterocycle may be linked to another group via any suitable atom, such as via a carbon or nitrogen atom. However, reference herein to piperidino or morpholino refers to a piperidin-1-yl or morpholin-4-yl ring that is linked via the ring nitrogen.

By “bridged ring systems” is meant ring systems in which two rings share more than two atoms, see for example Advanced Organic Chemistry, by Jerry March, 4^{th} Edition, Wiley Interscience, pages 131-133, 1992. Examples of bridged heterocyclyl ring systems include, aza-bicyclo[2.2.1]heptane, 2-oxa-5-azabicyclo[2.2.1]heptane, aza-bicyclo[2.2.2]octane, aza-bicyclo[3.2.1]octane and quinuclidine.

By “spiro bicyclic ring systems” we mean that the two ring systems share one common spiro carbon atom, i.e. the heterocyclic ring is linked to a further carbocyclic or heterocyclic ring through a single common spiro carbon atom. Examples of spiro ring systems include 6-azaspiro[3.4]octane, 2-oxa-6-azaspiro[3.4]octane, 2-azaspiro[3.3]heptanes, 2-oxa-6-azaspiro[3.3]heptanes, 7-oxa-2-azaspiro[3.5]nonane, 6-oxa-2-azaspiro[3.4]octane, 2-oxa-7-azaspiro[3.5]nonane and 2-oxa-6-azaspiro[3.5]nonane.

The term “heteroaryl” or “heteroaromatic” means an aromatic mono-, bi-, or polycyclic ring incorporating one or more (for example 1-4, particularly 1, 2 or 3) heteroatoms selected from nitrogen, oxygen or sulfur. The term heteroaryl includes both monovalent species and divalent species. Examples of heteroaryl groups are monocyclic and bicyclic groups containing from five to twelve ring members, and more usually from five to ten ring members.

The heteroaryl group can be, for example, a 5- or 6-membered monocyclic ring or a 9- or 10-membered bicyclic ring, for example a bicyclic structure formed from fused five and six membered rings or two fused six membered rings. Each ring may contain up to about four heteroatoms typically selected from nitrogen, sulfur and oxygen. Typically, the heteroaryl ring will contain up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general, the number of basic nitrogen atoms present in the heteroaryl group, including any amino group substituents of the ring, will be less than five.

Examples of heteroaryl include furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, benzofuranyl, indolyl, isoindolyl, benzothienyl, benzoxazolyl, benzimidazolyl, benzothiazolyl, benzothiazolyl, indazolyl, purinyl, benzofurazanyl, quinolyl, isoquinolyl, quinazolinyl, quinoxalinyl, cinnolinyl, pteridinyl, naphthyridinyl, carbazolyl, phenazinyl, benzisoquinolinyl, pyridopyrazinyl, thieno[2,3b]-furanyl-, 2H-furo[3,2b]-pyranyl-, 5H-pyrido[2,3-d]-ooxazinyl-, 1H-pyrazolo[4,3-d]-oxazolyl, 4H-imidazo[4,5d]thiazolyl, pyrazino[2,3d]pyridazinyl, -imidazo[2,1b]thiazolyl, -imidazo[1,2b][1,2,4]-triazinyl. “Heteroaryl” also covers partially aromatic bi- or polycyclic ring systems wherein at least one ring is an aromatic ring and one or more of the other ring(s) is a nonaromatic, saturated or partially saturated ring, provided at least one ring contains one or more heteroatoms selected from nitrogen, oxygen or -sulfur-. Examples of partially aromatic heteroaryl groups include for example, tetrahydroisoquinolinyl, tetrahydroquinolinyl, 2-oxo-1,2,3,4-tetrahydroquinolinyl, dihydrobenzthienyl, dihydrobenzfuranyl, 2,3-dihydro-benzo[1,4]dioxinyl, benzo[1,3]dioxolyl, 2,2-dioxo-1,3-dihydro-2-benzothienyl, 4,5,6,7-tetrahydrobenzofuranyl, indolinyl, 1,2,3,4-tetrahydro-1,8-naphthyridinyl, 1,2,3,4-tetrahydropyrido[2,3-b]pyrazinyl, 3,4-dihydro-2H-pyrido[3,2-b][1,4]oxazinyl and 6,8-dihydro-5H-[1,2,4]triazolo[4,3-a]pyrazinyl.

Examples of five membered heteroaryl groups include but are not limited to pyrrolyl, furanyl, thienyl, imidazolyl, furazanyl, oxazolyl, oxadiazolyl, oxatriazolyl, isoxazolyl, thiazolyl, isothiazolyl, pyrazolyl, triazolyl and tetrazolyl groups.

Examples of six membered heteroaryl groups include but are not limited to pyridyl, pyrazinyl, pyridazinyl, pyrimidinyl and triazinyl.

A bicyclic heteroaryl group may be, for example, a group selected from: a benzene ring fused to a 5- or 6-membered ring containing 1, 2 or 3 ring heteroatoms; a pyridine ring fused to a 5- or 6-membered ring containing 1, 2 or 3 ring heteroatoms; a pyrimidine ring fused to a 5- or 6-membered ring containing 1 or 2 ring heteroatoms; a pyrrole ring fused to a 5- or 6-membered ring containing 1, 2 or 3 ring heteroatoms; a pyrazole ring fused to a 5- or 6-membered ring containing 1 or 2 ring heteroatoms; a pyrazine ring fused to a 5- or 6-membered ring containing 1 or 2 ring heteroatoms; an imidazole ring fused to a 5- or 6-membered ring containing 1 or 2 ring heteroatoms; an oxazole ring fused to a 5- or 6-membered ring containing 1 or 2 ring heteroatoms; an isoxazole ring fused to a 5- or 6-membered ring containing 1 or 2 ring heteroatoms; a thiazole ring fused to a 5- or 6-membered ring containing 1 or 2 ring heteroatoms; an isothiazole ring fused to a 5- or 6-membered ring containing 1 or 2 ring heteroatoms; a thiophene ring fused to a 5- or 6-membered ring containing 1, 2 or 3 ring heteroatoms; a furan ring fused to a 5- or 6-membered ring containing 1, 2 or 3 ring heteroatoms; a cyclohexyl ring fused to a 5- or 6-membered heteroaromatic ring containing 1, 2 or 3 ring heteroatoms; and a cyclopentyl ring fused to a 5- or 6-membered heteroaromatic ring containing 1, 2 or 3 ring heteroatoms.

Particular examples of bicyclic heteroaryl groups containing a six membered ring fused to a five membered ring include but are not limited to benzfuranyl, benzthiophenyl, benzimidazolyl, benzoxazolyl, benzisoxazolyl, benzthiazolyl, benzisothiazolyl, isobenzofuranyl, indolyl, isoindolyl, indolizinyl, indolinyl, isoindolinyl, purinyl (e.g., adeninyl, guaninyl), indazolyl, benzodioxolyl and pyrazolopyridinyl groups.

Particular examples of bicyclic heteroaryl groups containing two fused six membered rings include but are not limited to quinolinyl, isoquinolinyl, chromanyl, thiochromanyl, chromenyl, isochromenyl, chromanyl, isochromanyl, benzodioxanyl, quinolizinyl, benzoxazinyl, benzodiazinyl, pyridopyridinyl, quinoxalinyl, quinazolinyl, cinnolinyl, phthalazinyl, naphthyridinyl and pteridinyl groups.

The term “aryl” means a cyclic or polycyclic aromatic ring having from 5 to 12 carbon atoms. The term aryl includes both monovalent species and divalent species. Examples of aryl groups include, but are not limited to, phenyl, biphenyl, naphthyl and the like. In particular embodiment, an aryl is phenyl.

This specification also makes use of several composite terms to describe groups comprising more than one functionality. Such terms will be understood by a person skilled in the art. For Example heterocyclyl(m-nC)alkyl comprises (m-nC)alkyl substituted by heterocyclyl.

The term “aryl(1-2C)alkyl” means an aryl group covalently attached to a (1-2C)alkylene group, both of which are defined herein. Examples of aryl-(1-2C)alkyl groups include benzyl, phenylethyl, and the like.

“Heteroaryl(1-3C)alkyl” means a heteroaryl group covalently attached to a (1-3C)alkylene group, both of which are defined herein. Examples of heteroaryl-alkyl groups include pyridin-3-ylmethyl, 2-(benzofuran-2-yl)ethyl, and the like.

“Heterocyclyl(1-2C)alkyl” means a heterocyclyl group covalently attached to a (1-2C)alkylene group, both of which are defined herein.

“(3-6C)cycloalkyl-(1-2C)alkyl” means a (3-6C)cycloalkyl group covalently attached to a (1-2C)alkylene group, both of which are defined herein.

The term “optionally substituted” refers to either groups, structures, or molecules that are substituted and those that are not substituted. The term “wherein a/any CH, CH₂, CH₃ group or heteroatom (i.e. NH) within a R¹ group is optionally substituted” suitably means that (any) one of the hydrogen radicals of the R¹ group is substituted by a relevant stipulated group.

Where optional substituents are chosen from “one or more” groups it is to be understood that this definition includes all substituents being chosen from one of the specified groups or the substituents being chosen from two or more of the specified groups.

A wavy bond ( [symbol] ) is used herein to show a point of attachment.

The phrase “compound of the invention” means those compounds which are disclosed herein, both generically and specifically.

As used herein by itself or in conjunction with another term or terms, “pharmaceutically acceptable” refers to materials that are generally chemically and/or physically compatible with other ingredients (such as, for example, with reference to a formulation), and/or are generally physiologically compatible with the recipient (such as, for example, a subject) thereof.

As used herein by themselves or in conjunction with another term or terms, “subject(s)” and “patient(s)”, suitably refer to mammals, in particular humans.

### Compounds of the Invention

In a first aspect, a compound of formula I shown below, or a pharmaceutically acceptable salt thereof:

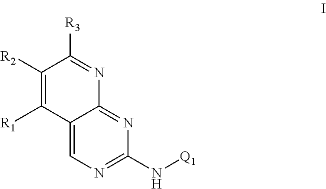

wherein:
R₁ is selected from hydrogen, (2C)alkynyl, (2C)alkenyl or (1-2C)alkyl;
R₂ is selected from hydrogen, halo, cyano, (1-2C)alkyl or a group:

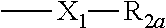

wherein:
   X¹ is selected from the group consisting of —O—, —C(O)—, —C(O)—O—, —O—C(O)—, —S(O)₀₋₂—, —C(O)—N(R_{2b})—, —N(R_{2b})—C(O)—, —NR_{2b}—, —SO₂N(R_{2b})—, or —N(R_{2b})SO₂—,
   where R₁₄ is independently selected from the group consisting of hydrogen or (1-2C)alkyl; and
   R₂ₐ is selected from hydrogen or (1-2C)alkyl, phenyl;
R₃ is hydrogen, halo, cyano or a group of the formula

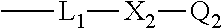

wherein:
L₁ is absent or (1-3C)alkylene;
X₂ is absent or is selected from the group consisting of —O—, —C(O)—, —NR₃ₐ—, —C(O)—O—, —O—C(O)—, —S(O)₀₋₂—, —C(O)—N(R₃ₐ)—, —N(R₃ₐ)—C(O)—, —O—C(O)—N(R₃ₐ)—, —N(R_{3b})—C(O)—O—, —N(R_{3b})—C(O)—NR₃ₐ—, —SO₂N(R₃ₐ)—, or —N(R₃ₐ)SO₂—, where R₃ₐ and R_{3b} are independently selected from the group consisting of hydrogen or (1-4C)alkyl;
Q₂ is selected from the group consisting of hydrogen, (1-6C)alkyl, (3-8C)cycloalkyl, (3-8C)cycloalkyl(1-4C)alkyl, aryl, aryl(1-4C)alkyl, heterocyclyl, heterocyclyl(1-4C)alkyl, heteroaryl and heteroaryl(1-4C)alkyl, wherein any alkyl, cycloalkyl, cycloalkyl-alkyl, aryl, arylalkyl, heterocyclyl, heterocyclyl-alkyl, heteroaryl or heteroaryl-alkyl in Q₂ is optionally substituted with one or more R_{3c} groups; and
each R_{3c} group present is independently selected from the group consisting of hydroxy, cyano, oxo, halogen, (1-6C)alkyl, (1-6C)alkoxy, (3-6C)cycloalkyl, or (3-6C)cycloalkyl(1-2C)alkyl, —NR_{3d}R₃ₑ, —C(O)—R_{3d}, —C(O)—OR_{3d}, —O—C(O)—R_{3d}, —C(O)—NR_{3d}R₃ₑ, —N(R₃ₑ)C(O)—R_{3d}, —S(O)₀₋₂R_{3d}—, —S(O)₂NR_{3d}R₃ₑ, —N(R₃ₑ)—S(O)₂R_{3d}, phenyl, 5 or 6-membered heteroaryl, or 4 to 6-membered heterocyclyl,
wherein R_{3d} and R₃ₑ are each independently hydrogen, (1-6C)alkyl, (3-6C)cycloalkyl, or (3-6C)cycloalkyl(1-2C)alkyl;
and wherein any alkyl, alkoxy, cycloalkyl, cycloalkyl-alkyl, phenyl, heteroaryl or heterocyclyl group present in a R₃, group, or any alkyl cycloalkyl, or cycloalkyl-alkyl group present in a R_{3d} or R₃ₑ group, is optionally further substituted by one or more substituents independently selected from hydroxy, cyano, halogen, —OR_{3f}, —NR_{3f}R_{3g} and —C(O)—R_{3f}, wherein R_{3f} and R_{3g} are both independently selected from hydrogen and (1-2C)alkyl; and
Q₁ is selected from hydrogen or:
   (i) a group of the formula:

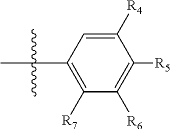

wherein:
   R₄ and R₆ are each independently selected from hydrogen, halo, cyano, nitro, or a group of the formula:

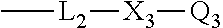

wherein:
L₂ is absent or (1-3C)alkylene;
X₃ is absent or is selected from the group consisting of —O—, —C(O)—, —NR₄ₐ—, —C(O)—O—, —O—C(O)—, —S(O)₀₋₂—, —C(O)—N(R₄ₐ)—, —N(R₄ₐ)—C(O)—, —O—C(O)—N(R₄ₐ)—, —N(R₄ₐ)—C(O)—O—, —N(R_{4b})—C(O)—NR₄ₐ—, —SO₂N(R₄ₐ)— or —N(R₄ₐ)SO₂—, where R₄ₐ and R_{4b} are independently selected from the group consisting of hydrogen or (1-4C)alkyl;
Q₃ is selected from the group consisting of hydrogen, (1-6C)alkyl, (3-8C)cycloalkyl, (3-8C)cycloalkyl(1-4C)alkyl, (2-6C)alkenyl, (2-6C)alkynyl, aryl, aryl(1-4C)alkyl, heterocyclyl, heterocyclyl(1-4C)alkyl, heteroaryl and heteroaryl(1-4C)alkyl, wherein any alkyl, cycloalkyl, cycloalkylalkyl, aryl, arylalkyl, heterocyclyl, heterocyclylalkyl, heteroaryl or heteroarylalkyl in Q₃ is optionally substituted with one or more R_{4c} groups; and
each R_{4c} group present is independently selected from the group consisting of hydroxy, cyano, oxo, halogen, (1-6C)alkyl, (1-6C)alkoxy, (3-6C)cycloalkyl, or (3-6C)cycloalkyl(1-2C)alkyl, —NR_{4d}R₄ₑ, —C(O)—R_{4d}, —C(O)—OR_{4d}, —O—C(O)—R_{4d}, —C(O)—NR_{4d}R₄ₑ, —N(R₄ₑ)C(O)—R_{4d}, —S(O)₀₋₂R_{4d}—, —S(O)₂NR_{4d}R₄ₑ, —N(R₄ₑ)—S(O)₂R_{4d}, phenyl, 5 or 6-membered heteroaryl, or 4 to 6-membered heterocyclyl,
wherein R_{4d} and R₄ₑ are each independently hydrogen, (1-6C)alkyl, (3-6C)cycloalkyl, or (3-6C)cycloalkyl(1-2C)alkyl;
and wherein any alkyl, alkoxy, cycloalkyl, cycloalkyl-alkyl, phenyl, heteroaryl or heterocyclyl group present in a R_{4c} group, or any alkyl, cycloalkyl or cycloalkyl-alkyl group present in a R_{4d} or R₄ₑ group, is optionally further substituted by one or more substituents independently selected from hydroxy, cyano, halogen, (1-2C)alkyl, —OR₄, —NR_{4f}R_{4g} and —C(O)—R_{4f}, wherein R_{4f} and R_{4g} are both independently selected from hydrogen and (1-2C)alkyl; R₅ is:
a) hydrogen, halo, cyano;
b) a group of the formula:

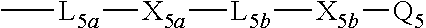

wherein:
L₅ₐ is absent or (1-3C)alkylene;
X₅ₐ is absent or is selected from the group consisting of —O—, —C(O)—, —NR₅ₐ₁—, —C(O)—O—, —O—C(O)—, —S(O)₀₋₂—, —C(O)—N(R₅ₐ₁)—, —N(R₅ₐ₁)—C(O)—, —O—C(O)—N(R₅ₐ₁), —N(R₅ₐ₁)—C(O)—O—, —N(R₅ₐ₂)—C(O)—NR₅ₐ₁—, —SO₂N(R₅ₐ₁)— or —N(R₅ₐ₁)SO₂—, where R₅ₐ₁ and R₅ₐ₂ are independently selected from the group consisting of hydrogen or (1-4C)alkyl;
L_{5b} is absent or (1-4C)alkylene;
X_{5b} is absent or is selected from the group consisting of —O—, —C(O)—, —NR_{5b1}—, —C(O)—O—, —O—C(O)—, —S(O)₀₋₂—, —C(O)—N(R_{5b1})—, —N(R_{5b1})—C(O)—, —O—C(O)—N(R_{5b1})—, —N(R_{5b1})—C(O)—O—, —N(R_{5b2})—C(O)—NR_{5b1}—, —SO₂N(R_{5b1})— or —N(R_{5b1})SO₂—, where R_{5b1} and R_{5b2} are independently selected from the group consisting of hydrogen or (1-4C)alkyl; Q₅ is selected from the group consisting of hydrogen, (1-6C)alkyl, (3-8C)cycloalkyl, (3-8C)cycloalkyl(1-4C)alkyl, phenyl, phenyl(1-2C)alkyl, 3 to 8-membered heterocyclyl, [3 to 8-membered heterocyclyl](1-2C)alkyl, 5 or 6-membered heteroaryl and 5 or 6-membered heteroaryl(1-2C)alkyl and wherein Q₅ is optionally substituted with one or more R_{5c} groups;
and each R₅, group present is independently selected from the group consisting of hydroxy, cyano, oxo, halogen, (1-6C)alkyl, (1-6C)alkoxy, (3-8C)cycloalkyl (including spiro-fused (3-8C)cycloalkyl), or (3-8C)cycloalkyl(1-2C)alkyl, —NR_{5d}R₅ₑ, —C(O)—R_{5d}, —C(O)—OR_{5d}, —O—C(O)—R_{5d}, —C(O)—NR_{5d}R₅ₑ, —N(R₅ₑ)C(O)—R_{5d}, —S(O)₀₋₂R_{5d}—, —S(O)₂NR_{5d}R₅ₑ, —N(R₅ₑ)—S(O)₂R_{5d}, phenyl, 5 or 6-membered heteroaryl, or 4 to 8-membered heterocyclyl,
wherein R_{5d} and R₅ₑ are each independently hydrogen, (1-6C)alkyl, (3-6C)cycloalkyl, or (3-6C)cycloalkyl(1-2C)alkyl; and wherein any alkyl, alkoxy, cycloalkyl, cycloalkyl-alkyl, phenyl, heteroaryl or heterocyclyl group present in a R₅, group, or any alkyl, cycloalkyl or cycloalkyl-alkyl group present in a R_{5d} or R₅ₑ group, is optionally further substituted by one or more substituents independently selected from hydroxy, cyano, halo, R_{5f}, —OR_{5f}, —NR_{5f}R_{5g} and —C(O)—R_{5f}, wherein R_{5f} and R_{5g} are both independently selected from hydrogen, (1-4C)alkyl, hydroxy(1-4C)alkyl, halo(1-4C)alkyl, or (1-2C)alkoxy(1-4C)alkyl;
R₇ is selected from hydrogen, halogen, hydroxy, methyl, hydroxymethyl, methoxy, —CF₃, —OCF₃ or cyano;
or R₄ and R₅, or R₅ and R₆ are linked to form a fused phenyl, 5- or 6-membered heteroaryl or 5 to 8-membered heterocyclic ring, which is optionally substituted by one or more substituent groups selected from hydroxy, cyano, halo, (1-2C)alkyl, (1-2C)haloalkyl, (1-2C)alkoxy or (1-2C)haloalkoxy;
(ii) a group of the formula:

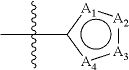

wherein:
   A₁ is selected from N, NR_{1N}, O, S or CR₇;
   A₂ is selected from N, NR_{1N}, O, S or CR₄;
   A₃ is selected from N, NR_{1N}, O, S or CR₅;
   A₄ is selected from N, NR_{1N}, O, S or CR₇;
   R_{1N} is selected from hydrogen, (1-4C)alkyl, (2-4C)alkanoyl, phenyl, benzyl, (3-8C)cycloalkyl, (3-8C)cycloalkyl-(1-2C)alkyl, a carbon-linked 4 to 7 membered heterocyclyl, or 4 to 7 membered heterocyclyl(1-2C)alkyl, and wherein any alkyl, alkanoyl, cycloalkyl, cycloalkylalkyl, phenyl, benzyl, heterocyclyl, heterocyclylalkyl group is optionally substituted with one or more R_{5c} groups defined above;
   R₄, R₅ and R₇ are as defined above;
   and with the proviso that:
      (i) only one of A₁, A₂, A₃ and A₄ can be O or S;
      (ii) only one of A₁, A₂, A₃ and A₄ can be NR_{1N};
      (iii) 1 to 4 of A₁, A₂, A₃ and A₄ can be N;
(iii) a group of the formula:

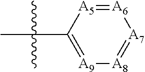

wherein:
A₅ is selected from N or CH;
A₆ is selected from N or CR₄;
A₇ is selected from N or CR₅;
A₈ is selected from N or CR₅;
A₉ is selected from N or CR₇;
R₄, R₅, R₆ and R₇ are as defined above;
and with the proviso that one or two of A₅, A₆, A₇, A₈ and A₉ can be N;
(iv) a group of the formula:

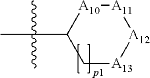

wherein:
p1 is 0 or 1;
A₁₀ is selected from NH, CH₂ or CHR₇;
A₁₁ is selected from NR_{4N}, CH₂ or CHR₄;
A₁₂ is selected from NR_{5N}, CH₂ or CHR₅;
A₁₃ is selected from NR_{6N}, CH₂ or CHR₆;
R₄, R₅, R₆ and R₇ are as defined above;
R_{4N} and R_{6N} are each independently selected from hydrogen or a group of the formula:

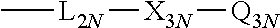

wherein:
L_{2N} is absent or (1-3C)alkylene;
X_{3N} is absent or is selected from the group consisting of:
   (i) —C(O)—, —S(O)₀₋₂— or —C(O)—N(R₄ₐ)— when L₂ is absent; or
   (ii) —O—, —C(O)—, —NR₄ₐ—, —C(O)—O—, —O—C(O)—, —S(O)₀₋₂—, —C(O)—N(R₄ₐ)—, —N(R₄ₐ)—C(O)—, —O—C(O)—N(R₄ₐ)—, —N(R₄)—C(O)—O—, —N(R_{4b})—C(O)—NR₄ₐ—, —SO₂N(R₄ₐ)— or —N(R₄ₐ)SO₂— when L₂ is (1-3C)alkylene;
   wherein R₄ₐ and R_{4b} are as defined above;
Q_{3N} is selected from the group consisting of hydrogen, (1-6C)alkyl, (3-8C)cycloalkyl, (3-8C)cycloalkyl(1-4C)alkyl, (2-6C)alkenyl, (2-6C)alkynyl, aryl, aryl(1-4C)alkyl, heterocyclyl, heterocyclyl(1-4C)alkyl, heteroaryl and heteroaryl(1-4C)alkyl, wherein any alkyl, cycloalkyl, cycloalkylalkyl, aryl, arylalkyl, heterocyclyl, heterocyclylalkyl, heteroaryl or heteroarylalkyl in Q₃ is optionally substituted with one or more R_{4c} groups; and each R₄ₑ group present is as defined above;
R_{5N} is:
   a) hydrogen;
   b) a group of the formula:

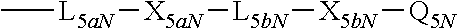

wherein:
L_{5aN} is absent or (1-3C)alkylene;
X_{5aN} is absent or is selected from the group consisting of:
(i) —C(O)—, —S(O)₀₋₂—, —C(O)—N(R₅ₐ₁)— when L_{5aN} is absent; or
(ii) —O—, —C(O)—, —NR₅ₐ₁—, —C(O)—O—, —O—C(O)—, —S(O)₀₋₂—, —C(O)—N(R₅ₐ₁), —N(R₅ₐ₁)C(O)—, —O—C(O)—N(R₅ₐ₁), —N(R₅ₐ)C(O)—O—, —N(R₅ₐ₂)—C(O)—NR₅ₐ₁—, —SO₂N(R₅ₐ)— or —N(R₅ₐ₁)SO₂— when L_{5aN} is present;
where R₅ₐ₁ and R₅ₐ₂ are as defined above;
L_{5bN} is absent or (1-40)alkylene;
X_{5bN} is absent or is selected from the group consisting of —O—, —C(O)—, —NR_{5b1}—, —C(O)—O—, —O—C(O)—, —S(O)₀₋₂—, —C(O)—N(R_{5b})—, —N(R_{5b})—C(O)—, —O—C(O)—N(R_{5b})—, —N(R_{5b1})—C(O)—O—, —N(R_{5b2})—C(O)—NR_{5b1}—, —SO₂N(R_{5b1})— or —N(R_{5b1})SO₂—, where R_{5b1} and R_{5b2} are independently selected from the group consisting of hydrogen or (1-4C)alkyl;
Q_{5N} is selected from the group consisting of:
(i) hydrogen;
(ii) (1-6C)alkyl, (3-8C)cycloalkyl, (3-8C)cycloalkyl(1-4C)alkyl, phenyl, phenyl(1-4C)alkyl, 5- or 6-membered heteroaryl, 5- or 6-membered heteroaryl(1-2C)alkyl;
(iii) a carbon-linked 4 to 8 membered heterocyclyl (when -L_{5aN}, X_{5aN}, L_{5bN}, and X_{5bN} are absent) or a 4 to 8 membered heterocyclyl (when one or more of -L_{5aN}, X_{5aN}, L_{5bN}, and X_{5bN} are present), or [4 to 8 membered heterocyclyl](1-2C)alkyl;
and wherein Q_{5N} is optionally substituted with one or more R_{5c} groups as defined above;
or R_{4N} and R_{5N}, or R_{5N} and R_{6N} are linked to form a fused phenyl, 5- or 6-membered heteroaryl or 5 to 8-membered heterocyclic ring, which is optionally substituted by one or more substituent groups selected from hydroxy, cyano, halo, (1-2C)alkyl, (1-2C)haloalkyl, (1-2C)alkoxy or (1-2C)haloalkoxy;
and with the proviso that one or two of A₁₀, A₁₁, A₁₂ and A₁₃ can be N;
(v) a group of the formula:

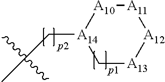

wherein:
p1, A₁₀, A₁₁, A₁₂, and A₁₃ are as defined above;
p2 is 1 to 6;
A₁₄ is C or N;
and with the proviso that one of A₁₀, A₁₁, A₁₂ and A₁₃ can be N.

In a further aspect, the present invention provides a compound of formula I shown below, or a pharmaceutically acceptable salt thereof:

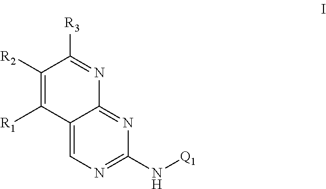

wherein:
R₁ is selected from hydrogen, (2C)alkynyl, (2C)alkenyl or (1-2C)alkyl;
R₂ is selected from hydrogen, halo, cyano, (1-2C)alkyl or a group:

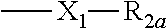

wherein:
   X¹ is selected from the group consisting of —O—, —C(O)—, —C(O)—O—, —O—C(O)—, —S(O)₀₋₂—, —C(O)—N(R_{2b})—, —N(R_{2b})—C(O)—, —NR_{2b}—, —SO₂N(R_{2b})—, or —N(R_{2b})SO₂—, where R_{2b} is independently selected from the group consisting of hydrogen or (1-2C)alkyl; and
   R₂ₐ is selected from hydrogen or (1-2C)alkyl, phenyl;
R₃ is hydrogen, halo, cyano or a group of the formula

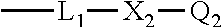

wherein:
   L₁ is absent or (1-3C)alkylene;
   X₂ is absent or is selected from the group consisting of —O—, —C(O)—, —NR₃ₐ—, —C(O)—O—, —O—C(O)—, —S(O)₀₋₂—, —C(O)—N(R₃ₐ)—, —N(R₃ₐ)—C(O)—, —O—C(O)—N(R₃ₐ)—, —N(R_{3b})—C(O)—O—, —N(R_{3b})—C(O)—NR₃ₐ—, —SO₂N(R₃ₐ)—, or —N(R₃ₐ)SO₂—, where R₃ₐ and R_{3b} are independently selected from the group consisting of hydrogen or (1-4C)alkyl;
   Q₂ is selected from the group consisting of hydrogen, (1-6C)alkyl, (3-8C)cycloalkyl, (3-8C)cycloalkyl(1-4C)alkyl, aryl, aryl(1-4C)alkyl, heterocyclyl, heterocyclyl(1-4C)alkyl, heteroaryl and heteroaryl(1-4C)alkyl, wherein any alkyl, cycloalkyl, cycloalkyl-alkyl, aryl, arylalkyl, heterocyclyl, heterocyclyl-alkyl, heteroaryl or heteroaryl-alkyl in Q₂ is optionally substituted with one or more R_{3c} groups; and
   each R₃ₑ group present is independently selected from the group consisting of hydroxy, cyano, oxo, halogen, (1-6C)alkyl, (1-6C)alkoxy, (3-6C)cycloalkyl, or (3-6C)cycloalkyl(1-2C)alkyl, —NR_{3d}R₃ₑ, —C(O)—R_{3d}, —C(O)—OR_{3d}, —O—C(O)—R_{3d}, —C(O)—NR_{3d}R₃ₑ, —N(R₃ₑ)C(O)—R_{3d}, —S(O)₀₋₂R_{3d}—, —S(O)₂NR_{3d}R₃ₑ, —N(R₃ₑ)—S(O)₂R_{3d}, phenyl, 5 or 6-membered heteroaryl, or 4 to 6-membered heterocyclyl,
   wherein R_{3d} and R₃ₑ are each independently hydrogen, (1-6C)alkyl, (3-6C)cycloalkyl, or (3-6C)cycloalkyl(1-2C)alkyl;
   and wherein any alkyl, alkoxy, cycloalkyl, cycloalkyl-alkyl, phenyl, heteroaryl or heterocyclyl group present in a R_{3c} group, or any alkyl cycloalkyl, or cycloalkyl-alkyl group present in a R_{3d} or R₃ₑ group, is optionally further substituted by one or more substituents independently selected from hydroxy, cyano, halogen, —OR₃ₐ, —NR_{3f}R_{3g} and —C(O)—R_{3f}, wherein R₃ₜ and Rag are both independently selected from hydrogen and (1-2C)alkyl; and
Q₁ is selected from hydrogen or:
   (i) a group of the formula:

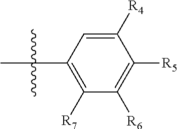

wherein:
   R₄, R₅ and R₇ are each independently selected from hydrogen, halo, cyano, nitro, or a group of the formula:

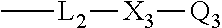

wherein:
L₂ is absent or (1-3C)alkylene;
X₃ is absent or is selected from the group consisting of —O—, —C(O)—, —NR₄ₐ—, —C(O)—O—, —O—C(O)—, —S(O)₀₋₂—, —C(O)—N(R₄ₐ)—, —N(R₄ₐ)—C(O)—, —O—C(O)—N(R₄ₐ)—, —N(R₄ₐ)—C(O)—O—, —N(R_{4b})—C(O)—NR₄ₐ—, —SO₂N(R₄ₐ)— or —N(R₄ₐ)SO₂—, where R₄ₐ and R_{4b} are independently selected from the group consisting of hydrogen or (1-4C)alkyl;
Q₃ is selected from the group consisting of hydrogen, (1-6C)alkyl, (3-8C)cycloalkyl, (3-8C)cycloalkyl(1-4C)alkyl, (2-6C)alkenyl, (2-6C)alkynyl, aryl, aryl(1-4C)alkyl, heterocyclyl, heterocyclyl(1-4C)alkyl, heteroaryl and heteroaryl(1-4C)alkyl, wherein any alkyl, cycloalkyl, cycloalkylalkyl, aryl, arylalkyl, heterocyclyl, heterocyclylalkyl, heteroaryl or heteroarylalkyl in Q₃ is optionally substituted with one or more R_{4c} groups; and
each R_{4c} group present is independently selected from the group consisting of hydroxy, cyano, oxo, halogen, (1-6C)alkyl, (1-6C)alkoxy, (3-6C)cycloalkyl, or (3-6C)cycloalkyl(1-2C)alkyl, —NR_{4d}R₄ₑ, —C(O)—R_{4d}, —C(O)—OR_{4d}, —O—C(O)—R_{4d}, —C(O)—NR_{4d}R₄ₑ, —N(R₄ₑ)C(O)—R_{4d}, —S(O)₀₋₂R_{4d}—, —S(O)₂NR_{4d}R₄ₑ, —N(R₄ₑ)—S(O)₂R_{4d}, phenyl, 5 or 6-membered heteroaryl, or 4 to 6-membered heterocyclyl,
wherein R_{4d} and R₄ₑ are each independently hydrogen, (1-6C)alkyl, (3-6C)cycloalkyl, or (3-6C)cycloalkyl(1-2C)alkyl;
and wherein any alkyl, alkoxy, cycloalkyl, cycloalkyl-alkyl, phenyl, heteroaryl or heterocyclyl group present in a R_{4c} group, or any alkyl, cycloalkyl or cycloalkyl-alkyl group present in a R_{4d} or R₄ₑ group, is optionally further substituted by one or more substituents independently selected from hydroxy, cyano, halogen, (1-2C)alkyl, —OR_{4f}, —NR_{4f}R_{4g}, —C(O)OR_{4f}, —OC(O)R_{4f}, —C(O)NR_{4f}R_{4g}, —NR_{4g}C(O)R_{4f}, —S(O)₀₋₂—R_{4f} and —C(O)—R_{4f}, wherein R_{4f} and R_{4g} are both independently selected from hydrogen and (1-2C)alkyl; R₅ is:
a) hydrogen, halo, cyano;
b) a group of the formula:

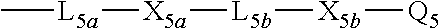

wherein:
L₅ₐ is absent or (1-3C)alkylene;
X₅ₐ is absent or is selected from the group consisting of —O—, —C(O)—, —NR₅ₐ₁—, —C(O)—O—, —O—C(O)—, —S(O)₀₋₂—, —C(O)—N(R₅ₐ₁)—, —N(R₅ₐ₁)—C(O)—, —O—C(O)—N(R₅ₐ), —N(R₅ₐ₁)—C(O)—O—, —N(R₅ₐ₂)—C(O)—NR₅ₐ₁—, —SO₂N(R₅ₐ₁)— or —N(R₅ₐ₁)SO₂—, where R₅ₐ₁ and R₅ₐ₂ are independently selected from the group consisting of hydrogen or (1-4C)alkyl;
L_{5b} is absent or (1-4C)alkylene;
X_{5b} is absent or is selected from the group consisting of —O—, —C(O)—, —NR_{5b1}—, —C(O)—O—, —O—C(O)—, —S(O)₀₋₂—, —C(O)—N(R_{5b1})—, —N(R_{5b1})—C(O)—, —C(O)—O—N(R_{5b1})—, —N(R_{5b1})—O—C(O)—, —N(R_{5b2})—C(O)—NR_{5b1}—, —SO₂N(R_{5b1})— or —N(R_{5b1})SO₂—, where R_{5b1} and R_{5b2} are independently selected from the group consisting of hydrogen or (1-4C)alkyl;
Q₅ is selected from the group consisting of hydrogen, (1-6C)alkyl, (3-8C)cycloalkyl, (3-8C)cycloalkyl(1-4C)alkyl, phenyl, phenyl(1-2C)alkyl, 3 to 8-membered heterocyclyl, [3 to 8-membered heterocyclyl](1-2C)alkyl, 5 or 6-membered heteroaryl and 5 or 6-membered heteroaryl(1-2C)alkyl and wherein Q₅ is optionally substituted with one or more R_{5c} groups;
and each R_{5c} group present is independently selected from the group consisting of hydroxy, cyano, oxo, halogen, (1-6C)alkyl, (1-6C)alkoxy, (3-8C)cycloalkyl (including spiro-fused (3-8C)cycloalkyl), or (3-8C)cycloalkyl(1-2C)alkyl, —NR_{5d}R₅ₑ, —C(O)—R_{5d}, —C(O)—OR_{5d}, —O—C(O)—R_{5d}, —C(O)—NR_{5d}R₅ₑ, —N(R₅ₑ)C(O)—R_{5d}, —S(O)₀₋₂R_{5d}—, —S(O)₂NR_{5d}R₅ₑ, —N(R₅ₑ)—S(O)₂R_{5d}, phenyl, 5 or 6-membered heteroaryl, or 4 to 8-membered heterocyclyl,
wherein R_{5d} and R₅ₑ are each independently hydrogen, (1-6C)alkyl, (3-6C)cycloalkyl, or (3-6C)cycloalkyl(1-2C)alkyl;
and wherein any alkyl, alkoxy, cycloalkyl, cycloalkyl-alkyl, phenyl, heteroaryl or heterocyclyl group present in a R_{5c} group, or any alkyl, cycloalkyl or cycloalkyl-alkyl group present in a R_{5d} or R₅ₑ group, is optionally further substituted by one or more substituents independently selected from hydroxy, cyano, halo, R_{5f}, —OR_{5f}, —NR_{5f}R_{5g} and —C(O)—R_{5f}, wherein R_{5f} and R_{5g} are both independently selected from hydrogen, (1-4C)alkyl, hydroxy(1-4C)alkyl, halo(1-4C)alkyl, or (1-2C)alkoxy(1-4C)alkyl;
or R₄ and R₅, or R₅ and R₆, or R₆ and R₇ are linked to form a fused phenyl, 5- or 6-membered heteroaryl or 5 to 8-membered heterocyclic ring, which is optionally substituted by one or more substituent groups selected from hydroxy, cyano, halo, (1-2C)alkyl, (1-2C)haloalkyl, (1-2C)alkoxy or (1-2C)haloalkoxy;
(ii) a group of the formula:

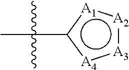

wherein:
A₁ is selected from N, NR_{1N}, O, S or CR₇;
A₂ is selected from N, NR_{1N}, O, S or CR₄;
A₃ is selected from N, NR_{1N}, O, S or CR₅;
A₄ is selected from N, NR_{1N}, O, S or CR₇;
R_{1N} is selected from hydrogen, (1-4C)alkyl, (2-4C)alkanoyl, phenyl, benzyl, (3-8C)cycloalkyl, (3-8C)cycloalkyl-(1-2C)alkyl, a carbon-linked 4 to 7 membered heterocyclyl, or 4 to 7 membered heterocyclyl(1-2C)alkyl, and wherein any alkyl, alkanoyl, cycloalkyl, cycloalkylalkyl, phenyl, benzyl, heterocyclyl, heterocyclylalkyl group is optionally substituted with one or more R_{5c} groups defined above;
R₄, R₅ and R₇ are as defined above;
and with the proviso that:
   (i) only one of A₁, A₂, A₃ and A₄ can be O or S;
   (ii) only one of A₁, A₂, A₃ and A₄ can be NR_{1N};
   (iii) 1 to 4 of A₁, A₂, A₃ and A₄ can be N;
(iii) a group of the formula:

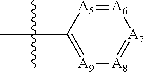

wherein:
A₅ is selected from N or CH;
A₉ is selected from N or CR₄;
A₇ is selected from N or CR₅;
A₈ is selected from N or CR₅;
A₉ is selected from N or CR₇;
R₄, R₅, R₆ and R₇ are as defined above;
and with the proviso that one or two of A₅, A₆, A₇, A₈ and A₉ can be N;
(iv) a group of the formula:

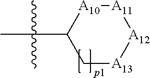

wherein:
p1 is 0 or 1;
A₁₀ is selected from NH, CH₂ or CHR₇;
A₁₁ is selected from NR_{4N}, CH₂ or CHR₄;
A₁₂ is selected from NR_{5N}, CH₂ or CHR₅;
A₁₃ is selected from NR_{6N}, CH₂ or CHR₆;
R₄, R₅, R₆ and R₇ are as defined above;
R_{4N} and R_{6N} are each independently selected from hydrogen or a group of the formula:

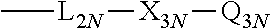

wherein:
L_{2N} is absent or (1-3C)alkylene;
X_{3N} is absent or is selected from the group consisting of:
   (i) —C(O)—, —S(O)₀₋₂— or —C(O)—N(R₄ₐ)— when L₂ is absent; or
   (ii) —O—, —C(O)—, —NR₄ₐ—, —C(O)—O—, —O—C(O)—, —S(O)₀₋₂—, —C(O)—N(R₄ₐ)—, —N(R₄ₐ)—C(O)—, —O—C(O)—N(R₄ₐ)—, —N(R₄ₐ)—C(O)—O—, —N(R_{4b})—C(O)—NR₄ₐ—, —SO₂N(R₄ₐ)— or —N(R₄ₐ)SO₂— when L₂ is (1-3C)alkylene;
   wherein R₄ₐ and R_{4b} are as defined above;
Q_{3N} is selected from the group consisting of hydrogen, (1-6C)alkyl, (3-8C)cycloalkyl, (3-8C)cycloalkyl(1-4C)alkyl, (2-6C)alkenyl, (2-6C)alkynyl, aryl, aryl(1-4C)alkyl, heterocyclyl, heterocyclyl(1-4C)alkyl, heteroaryl and heteroaryl(1-4C)alkyl, wherein any alkyl, cycloalkyl, cycloalkylalkyl, aryl, arylalkyl, heterocyclyl, heterocyclylalkyl, heteroaryl or heteroarylalkyl in Q₃ is optionally substituted with one or more R_{4c} groups; and each R_{4c} group present is as defined above; R_{5N} is
   a) hydrogen;
   b) a group of the formula:

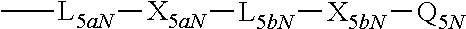

wherein:
L_{5aN} is absent or (1-3C)alkylene;
X_{5aN} is absent or is selected from the group consisting of:
(i) —C(O)—, —S(O)₀₋₂—, —C(O)—N(R₅ₐ₁)— when L_{5aN} is absent; or
(ii) —O—, —C(O)—, —NR₅ₐ₁—, —C(O)—O—, —O—C(O)—, —S(O)O-2—, —C(O)—N(R₅ₐ₁)—, —N(R₅ₐ₁)—C(O)—, —O—C(O)—N(R₅ₐ₁)—, —N(R₅ₐ)—C(O)—O—, —N(R₅ₐ₂)—C(O)—NR₅ₐ₁—, —SO₂N(R₅ₐ) or —N(R₅ₐ₁)SO₂— when L_{5aN} is present;
where R₅ₐ₁ and R₅ₐ₂ are as defined above;
L_{5bN} is absent or (1-40)alkylene;
X_{5bN} is absent or is selected from the group consisting of —O—, —C(O)—, —NR_{5b1}—, —C(O)—O—, —O—C(O)—, —S(O)₀₋₂—, —C(O)—N(R_{5ba})—, —N(R_{5b1})—C(O)—, —O—C(O)—N(R_{5b1})—, —N(R_{5b1})—C(O)—O—, —N(R_{5b2})—C(O)—NR_{5b1}—, —SO₂N(R_{5b1})— or —N(R_{5b1})SO₂—, where R_{5b1} and R_{5b2} are independently selected from the group consisting of hydrogen or (1-4C)alkyl;
Q_{5N} is selected from the group consisting of:
(i) hydrogen;
(ii) (1-6C)alkyl, (3-8C)cycloalkyl, (3-8C)cycloalkyl(1-4C)alkyl, phenyl, phenyl(1-4C)alkyl, 5- or 6-membered heteroaryl, 5- or 6-membered heteroaryl(1-2C)alkyl;
(iii) a carbon-linked 4 to 8 membered heterocyclyl (when -L_{5aN}, X_{5aN}, L_{5bN}, and X_{5bN} are absent) or a 4 to 8 membered heterocyclyl (when one or more of -L_{5aN}, X_{5aN}, L_{5bN}, and X_{5bN} are present), or [4 to 8 membered heterocyclyl](1-2C)alkyl;
and wherein Q_{5N} is optionally substituted with one or more R_{5c} groups as defined above;
or R_{4N} and R_{5N}, or R_{5N} and R_{6N} are linked to form a fused phenyl, 5- or 6-membered heteroaryl or 5 to 8-membered heterocyclic ring, which is optionally substituted by one or more substituent groups selected from hydroxy, cyano, halo, (1-2C)alkyl, (1-2C)haloalkyl, (1-2C)alkoxy or (1-2C)haloalkoxy;
and with the proviso that one or two of A₁₀, A₁₁, A₁₂ and A₁₃ can be N;
(v) a group of the formula:

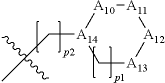

wherein:
p1, A₁₀, A₁₁, A₁₂, and A₁₃ are as defined above;
p2 is 1 to 6;
A₁₄ is C or N;
and with the proviso that one of A₁₀, A₁₁, A₁₂ and A₁₃ can be N.

In a particular group of compounds of the invention, the compounds have the structural formula I above, wherein R₃ is not hydrogen, i.e. R₃ is halo, cyano or a group of the formula

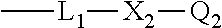

wherein:
L₁ is absent or (1-3C)alkylene;
X₂ is absent or is selected from the group consisting of —O—, —C(O)—, —NR₃ₐ—, —C(O)—O—, —O—C(O)—, —S(O)₀₋₂—, —C(O)—N(R₃ₐ)—, —N(R₃ₐ)—C(O)—, —O—C(O)—N(R₃ₐ)—, —N(R_{3b})—C(O)—O—, —N(R_{3b})—C(O)—NR₃ₐ—, —SO₂N(R₃ₐ)—, or —N(R₃ₐ)SO₂—, where R₃ₐ and R_{3b} are independently selected from the group consisting of hydrogen or (1-4C)alkyl;
Q₂ is selected from the group consisting of hydrogen, (1-6C)alkyl, (3-8C)cycloalkyl, (3-8C)cycloalkyl(1-4C)alkyl, aryl, aryl(1-4C)alkyl, heterocyclyl, heterocyclyl(1-4C)alkyl, heteroaryl and heteroaryl(1-4C)alkyl, wherein any alkyl, cycloalkyl, cycloalkyl-alkyl, aryl, arylalkyl, heterocyclyl, heterocyclyl-alkyl, heteroaryl or heteroaryl-alkyl in Q₂ is optionally substituted with one or more R_{3c} groups; and each R_{3c} group present is independently selected from the group consisting of hydroxy, cyano, oxo, halogen, (1-6C)alkyl, (1-6C)alkoxy, (3-6C)cycloalkyl, (3-6C)cycloalkyl(1-2C)alkyl, —NR_{3d}R₃ₑ, —C(O)—R_{3d}, —C(O)—OR_{3d}, —O—C(O)—R_{3d}, —C(O)—NR_{3d}R₃ₑ, —N(R₃ₑ)C(O)—R_{3d}, —S(O)₀₋₂R_{3d}—, —S(O)₂NR_{3d}R₃ₑ, —N(R₃ₑ)—S(O)₂R_{3d}, phenyl, 5 or 6-membered heteroaryl, or 4 to 6-membered heterocyclyl,
wherein R_{3d} and R₃ₑ are each independently hydrogen, (1-6C)alkyl, (3-6C)cycloalkyl, or (3-6C)cycloalkyl(1-2C)alkyl;
and wherein any alkyl, alkoxy, cycloalkyl, cycloalkyl-alkyl, phenyl, heteroaryl or heterocyclyl group present in a R₃ₑ group, or any alkyl cycloalkyl, or cycloalkyl-alkyl group present in a R_{3d} or R₃ₑ group, is optionally further substituted by one or more substituents independently selected from hydroxy, cyano, halogen, —OR_{3f}, —NR_{3f}R₃ₐ and —C(O)—R_{3f}, wherein R₃ᵣ and R₃ₐ are both independently selected from hydrogen and (1-2C)alkyl.

Particular compounds of the invention include, for example, compounds of the formula I, or pharmaceutically acceptable salts, hydrates and/or solvates thereof, wherein, unless otherwise stated, each of R_{N}, R_{NA}, R₁, V₁, R₂, Q₁ and L each have any of the meanings defined hereinbefore or are as defined in any one of paragraphs (1) to (40) hereinafter:—
(1) R₁ is selected from ethynyl or ethenyl;
(2) R₁ is ethynyl;
(3) R₁ is ethenyl;
(4) R₂ is selected from hydrogen, halo, cyano, (1-2C)alkyl or a group:

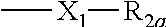

wherein:
   X¹ is selected from the group consisting of —O—, —C(O)—, —C(O)—O—, —O—C(O)—, —S(O)₀₋₂—, —C(O)—N(R_{2b})—, —N(R_{2b})—C(O)— or —NR_{2b}—;
   where R_{2b} is independently selected from the group consisting of hydrogen or (1-2C)alkyl; and
   R₂ₐ is selected from hydrogen, (1-2C)alkyl or phenyl.
(5) R₂ is selected from hydrogen, halo, cyano or (1-2C)alkyl.
(6) R₂ is selected from hydrogen or methyl.
(7) R₃ is hydrogen, halo, cyano or a group of the formula

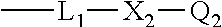

wherein:
L₁ is absent or (1-2C)alkylene;
X₂ is absent or is selected from the group consisting of —O—, —C(O)—, —NR₃ₐ—, —C(O)—O—, —O—C(O)—, —S(O)₀₋₂—, —C(O)—N(R₃ₐ)—, —N(R₃ₐ)—C(O)—, —O—C(O)—N(R₃ₐ)—, —N(R₃ₐ)—C(O)—O—, —N(R_{3b})—C(O)—NR₃ₐ—, —SO₂N(R₃ₐ)—, or —N(R₃ₐ)SO₂—, where R₃ₐ
and R_{3b} are independently selected from the group consisting of hydrogen or (1-2C)alkyl;
Q₂ is selected from the group consisting of hydrogen, (1-4C)alkyl, (3-6C)cycloalkyl, (3-6C)cycloalkyl(1-2C)alkyl, phenyl, phenyl(1-4C)alkyl, 3- to 10-membered heterocyclyl, [3- to 10-membered heterocyclyl](1-2C)alkyl, 5- to 10-membered heteroaryl and [5- to 10-membered heteroaryl](1-2C)alkyl, wherein any alkyl, cycloalkyl, cycloalkyl-alkyl, phenyl, phenylalkyl, heterocyclyl, heterocyclyl-alkyl, heteroaryl or heteroaryl-alkyl in Q₂ is optionally substituted with one or more R₃, groups; and
each R_{3c} group present is independently selected from the group consisting of hydroxy, cyano, oxo, halogen, (1-4C)alkyl, (1-4C)alkoxy, (3-6C)cycloalkyl, or (3-6C)cycloalkyl(1-2C)alkyl, —NR_{3d}R₃ₑ, —C(O)—R_{3d}, —C(O)—OR_{3d}, —O—C(O)—R_{3d}, —C(O)—NR_{3d}R₃ₑ, —N(R₃ₑ)C(O)—R_{3d}, —S(O)₀₋₂R_{3d}—, —S(O)₂NR_{3d}R₃ₑ, —N(R₃ₑ)—S(O)₂R_{3d}, phenyl, 5 or 6-membered heteroaryl, or 4 to 6-membered heterocyclyl,
wherein R_{3d} and R₃ₑ are each independently hydrogen, (1-4C)alkyl, (3-6C)cycloalkyl, or (3-6C)cycloalkyl(1-2C)alkyl;
and wherein any alkyl, alkoxy, cycloalkyl, cycloalkyl-alkyl, phenyl, heteroaryl or heterocyclyl group present in a R_{3c} group, or any alkyl, cycloalkyl, or cycloalkyl-alkyl group present in a R_{3d} or R₃ₑ group, is optionally further substituted by one or more substituents independently selected from hydroxy, cyano, halogen, (1-2C)alkyl, —OR_{3f}, —NR_{3f}R_{3g} and —C(O)—R_{3f}, wherein R_{3f} and R_{3g} are both independently selected from hydrogen and (1-2C)alkyl; R₃ is hydrogen, halo, cyano, or a group of the formula

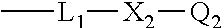

wherein:
L₁ is absent or (1-2C)alkylene;
X₂ is absent or is selected from the group consisting of —O—, —C(O)—, —NR₃ₐ—, —C(O)—O—, —O—C(O)—, —S(O)₀₋₂—, —C(O)—N(R₃ₐ)—, —N(R₃ₐ)—C(O)—, —N(R_{3b})—C(O)—O—, —N(R_{3b})—C(O)—NR₃ₐ—, —SO₂N(R₃ₐ)— or —N(R₃ₐ)SO₂—, where R₃ₐ is selected from the group consisting of hydrogen or (1-2C)alkyl;
Q₂ is selected from the group consisting of hydrogen, (1-4C)alkyl, (3-6C)cycloalkyl, (3-6C)cycloalkyl(1-2C)alkyl, phenyl, phenyl(1-4C)alkyl, 3- to 10-membered heterocyclyl, [3- to 10-membered heterocyclyl](1-2C)alkyl, 5- to 10-membered heteroaryl and [5- to 10-membered heteroaryl](1-2C)alkyl, wherein any alkyl, cycloalkyl, cycloalkyl-alkyl, phenyl, phenylalkyl, heterocyclyl, heterocyclyl-alkyl, heteroaryl or heteroaryl-alkyl in Q₂ is optionally substituted with one or more R₃, groups; and
each R_{3c} group present is independently selected from the group consisting of hydroxy, cyano, oxo, halogen, (1-4C)alkyl, (1-4C)alkoxy, (3-6C)cycloalkyl, or (3-6C)cycloalkyl(1-2C)alkyl, —NR_{3d}R₃ₑ, —C(O)—R_{3d}, —C(O)—OR_{3d}, —O—C(O)—R_{3d}, —C(O)—NR_{3d}R₃ₑ, —N(R₃ₑ)C(O)—R_{3d}, —S(O)₀₋₂R_{3d}—, —S(O)₂NR_{3d}R₃ₑ, N(R₃ₑ)—S(O)₂R_{3d}, phenyl, 5 or 6-membered heteroaryl, or 4 to 6-membered heterocyclyl,
wherein R_{3d} and R₃ₑ are each independently hydrogen or (1-2C)alkyl; and wherein any alkyl, alkoxy, cycloalkyl, cycloalkyl-alkyl, phenyl, heteroaryl or heterocyclyl group present in a R_{3c} group, or any alkyl group present in a R_{3d} or R₃ₑ group, is optionally further substituted by one or more substituents independently selected from hydroxy, cyano, halogen, (1-2C)alkyl, or —OR_{3f}, wherein R_{3f} is selected from hydrogen and (1-2C)alkyl.
(8) R₃ is halo, cyano, or a group of the formula

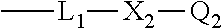

wherein:
L₁ is absent or (1-2C)alkylene;
X₂ is absent or is selected from the group consisting of —O—, —C(O)—, —NR₃ₐ—, —C(O)—O—, —O—C(O)—, —S(O)₀₋₂—, —C(O)—N(R₃ₐ)—, —N(R₃ₐ)—C(O)—, —O—C(O)—N(R₃ₐ)—, —N(R₃ₐ)—C(O)—O—, —N(R_{3b})—C(O)—NR₃ₐ—, —SO₂N(R₃ₐ)—, or —N(R₃ₐ)SO₂—, where R₃ₐ and R_{3b} are independently selected from the group consisting of hydrogen or (1-2C)alkyl;
Q₂ is selected from the group consisting of hydrogen, (1-4C)alkyl, (3-6C)cycloalkyl, (3-6C)cycloalkyl(1-2C)alkyl, phenyl, phenyl(1-4C)alkyl, 3- to 10-membered heterocyclyl, [3- to 10-membered heterocyclyl](1-2C)alkyl, 5- to 10-membered heteroaryl and [5- to 10-membered heteroaryl](1-2C)alkyl, wherein any alkyl, cycloalkyl, cycloalkyl-alkyl, phenyl, phenylalkyl, heterocyclyl, heterocyclyl-alkyl, heteroaryl or heteroaryl-alkyl in Q₂ is optionally substituted with one or more R₃, groups; and
each R_{3c} group present is independently selected from the group consisting of hydroxy, cyano, oxo, halogen, (1-4C)alkyl, (1-4C)alkoxy, (3-6C)cycloalkyl, or (3-6C)cycloalkyl(1-2C)alkyl, —NR_{3d}R₃ₑ, —C(O)—R_{3d}, —C(O)—OR_{3d}, —O—C(O)—R_{3d}, —C(O)—NR_{3d}R₃ₑ, —N(R₃ₑ)C(O)—R_{3d}, —S(O)₀₋₂R_{3d}—, —S(O)₂NR_{3d}R₃ₑ, —N(R₃ₑ)—S(O)₂R_{3d}, phenyl, 5 or 6-membered heteroaryl, or 4 to 6-membered heterocyclyl, wherein R_{3d} and R₃ₑ are each independently hydrogen, (1-4C)alkyl, (3-6C)cycloalkyl, or (3-6C)cycloalkyl(1-2C)alkyl;
and wherein any alkyl, alkoxy, cycloalkyl, cycloalkyl-alkyl, phenyl, heteroaryl or heterocyclyl group present in a R_{3c} group, or any alkyl, cycloalkyl, or cycloalkyl-alkyl group present in a R_{3d} or R₃ₑ group, is optionally further substituted by one or more substituents independently selected from hydroxy, cyano, halogen, (1-2C)alkyl, —OR_{3f}, —NRa_{f}R_{3g} and —C(O)—R_{3f}, wherein R_{3f} and R_{3g} are both independently selected from hydrogen and (1-2C)alkyl; R₃ is hydrogen, halo, cyano, or a group of the formula

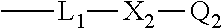

wherein:
L₁ is absent or (1-2C)alkylene;
X₂ is absent or is selected from the group consisting of —O—, —C(O)—, —NR₃ₐ—, —C(O)—O—, —O—C(O)—, —S(O)₀₋₂—, —C(O)—N(R₃ₐ)—, —N(R₃ₐ)—C(O)—, —N(R_{3b})—C(O)—O—, —N(R_{3b})—C(O)—NR₃ₐ—, —SO₂N(R₃ₐ)— or —N(R₃ₐ)SO₂—, where R₃ₐ is selected from the group consisting of hydrogen or (1-2C)alkyl;
Q₂ is selected from the group consisting of hydrogen, (1-4C)alkyl, (3-6C)cycloalkyl, (3-6C)cycloalkyl(1-2C)alkyl, phenyl, phenyl(1-4C)alkyl, 3- to 10-membered heterocyclyl, [3- to 10-membered heterocyclyl](1-2C)alkyl, 5- to 10-membered heteroaryl and [5- to 10-membered heteroaryl](1-2C)alkyl, wherein any alkyl, cycloalkyl, cycloalkyl-alkyl, phenyl, phenylalkyl, heterocyclyl, heterocyclyl-alkyl, heteroaryl or heteroaryl-alkyl in Q₂ is optionally substituted with one or more R_{3c} groups; and
each R_{3c} group present is independently selected from the group consisting of hydroxy, cyano, oxo, halogen, (1-4C)alkyl, (1-4C)alkoxy, (3-6C)cycloalkyl, or (3-6C)cycloalkyl(1-2C)alkyl, —NR_{3d}R₃ₑ, —C(O)—R_{3d}, —C(O)—OR_{3d}, —O—C(O)—R_{3d}, —C(O)—NR_{3d}R₃ₑ, —N(R₃ₑ)C(O)—R_{3d}, —S(O)₀₋₂R_{3d}—, —S(O)₂NR_{3d}R₃ₑ, —N(R₃ₑ)—S(O)₂R_{3d}, phenyl, 5 or 6-membered heteroaryl, or 4 to 6-membered heterocyclyl,
wherein R_{3d} and R₃ₑ are each independently hydrogen or (1-2C)alkyl;
and wherein any alkyl, alkoxy, cycloalkyl, cycloalkyl-alkyl, phenyl, heteroaryl or heterocyclyl group present in a R_{3c} group, or any alkyl group present in a R_{3d} or R₃ₑ group, is optionally further substituted by one or more substituents independently selected from hydroxy, cyano, halogen, (1-2C)alkyl, or —OR_{3f}, wherein R_{3f} is selected from hydrogen and (1-2C)alkyl.
(9) R₃ is hydrogen, cyano, or a group of the formula

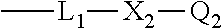

wherein:
L₁ is absent or (1-2C)alkylene;
X₂ is absent or is selected from the group consisting of —O—, —C(O)—, —NR₃ₐ—, —C(O)—O—, —O—C(O)—, —S(O)₀₋₂—, —C(O)—N(R₃ₐ)—, —N(R₃ₐ)—C(O)—, —N(R_{3b})—C(O)—O—, —N(R_{3b})—C(O)—NR₃ₐ—, —SO₂N(R₃ₐ)— or —N(R₃ₐ)SO₂—, where R₃ₐ is selected from the group consisting of hydrogen or (1-2C)alkyl;
Q₂ is selected from the group consisting of hydrogen, (1-4C)alkyl, (3-6C)cycloalkyl, (3-6C)cycloalkyl(1-2C)alkyl, phenyl, phenyl(1-4C)alkyl, 4- to 6-membered heterocyclyl, [4- to 6-membered heterocyclyl](1-2C)alkyl, 5- to 10-membered heteroaryl and [5- to 10-membered heteroaryl](1-2C)alkyl, wherein any alkyl, cycloalkyl, cycloalkyl-alkyl, phenyl, phenylalkyl, heterocyclyl, heterocyclyl-alkyl, heteroaryl or heteroaryl-alkyl in Q₂ is optionally substituted with one or more R₃ₑ groups; and
each R₃ₑ group present is independently selected from the group consisting of hydroxy, cyano, oxo, halogen, (1-4C)alkyl, (1-4C)alkoxy, —NR_{3d}R₃ₑ, —C(O)—R_{3d}, —C(O)—OR_{3d}, —O—C(O)—R_{3d}, —C(O)—NR_{3d}R₃ₑ, —N(R₃ₑ)C(O)—R_{3d}, —S(O)₀₋₂R_{3d}—, —S(O)₂NR_{3d}R₃ₑ, —N(R₃ₑ)—S(O)₂R_{3d}, phenyl, 5 or 6-membered heteroaryl, or 4 to 6-membered heterocyclyl,
wherein R_{3d} and R₃ₑ are each independently hydrogen or (1-2C)alkyl; and wherein any alkyl, alkoxy, cycloalkyl, cycloalkyl-alkyl, phenyl, heteroaryl or heterocyclyl group present in a R₃ₑ group is optionally further substituted by one or more substituents independently selected from hydroxy, cyano, halogen, (1-2C)alkyl, or —OR_{3f}, wherein R_{3f} is selected from hydrogen and (1-2C)alkyl;
(10) R₃ is hydrogen, cyano, or a group of the formula

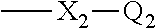

wherein:
X₂ is absent or is selected from the group consisting of —O—, —C(O)—, —NR₃ₐ—, —C(O)—O—, —O—C(O)—, —S(O)₀₋₂—, —C(O)—N(R₃ₐ)—, —N(R₃ₐ)—C(O)—, —N(R_{3b})—C(O)—O—, —N(R_{3b})—C(O)—NR₃ₐ—, —SO₂N(R₃ₐ)— or —N(R₃ₐ)SO₂—, where R₃ₐ is selected from the group consisting of hydrogen or (1-2C)alkyl;
Q₂ is selected from the group consisting of hydrogen, (1-4C)alkyl, (3-6C)cycloalkyl, (3-6C)cycloalkyl(1-2C)alkyl, phenyl, phenyl(1-4C)alkyl, 4- to 6-membered heterocyclyl, [4- to 6-membered heterocyclyl](1-2C)alkyl, 5- to 10-membered heteroaryl and [5- to 10-membered heteroaryl](1-2C)alkyl, wherein any alkyl, cycloalkyl, cycloalkyl-alkyl, phenyl, phenylalkyl, heterocyclyl, heterocyclyl-alkyl, heteroaryl or heteroaryl-alkyl in Q₂ is optionally substituted with one or more R₃, groups; and each R_{3c} group present is independently selected from the group consisting of hydroxy, cyano, oxo, halogen, (1-4C)alkyl, (1-4C)alkoxy, —NR_{3d}R₃ₑ, —C(O)—R_{3d}, —S(O)₀₋₂R_{3d}—, —S(O)₀₋₂R_{3d}—, phenyl, 5 or 6-membered heteroaryl, or 4 to 6-membered heterocyclyl,
wherein R_{3d} and R₃ₑ are each independently hydrogen or (1-2C)alkyl; and wherein any alkyl, alkoxy, phenyl, heteroaryl or heterocyclyl group present in a R₃, group, is optionally further substituted by one or more substituents independently selected from hydroxy, cyano, halogen, (1-2C)alkyl, or —OR_{3f}, wherein R_{3f} is selected from hydrogen and (1-2C)alkyl;
(11) R₃ is hydrogen, cyano, or a group of the formula

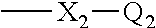

wherein:
X₂ is absent or is selected from the group consisting of —O—, —C(O)—, —NR₃ₐ—, —C(O)—O—, —S(O)₀₋₂—, —C(O)—N(R₃ₐ)—, —N(R₃ₐ)—C(O)—, —N(R_{3b})—C(O)—O—, —N(R_{3b})—C(O)—NR₃ₐ—, —SO₂N(R₃ₐ)— or —N(R₃,)SO₂—, where R₃ₐ is selected from the group consisting of hydrogen or methyl;
Q₂ is selected from the group consisting of hydrogen, (1-3C)alkyl, (3-6C)cycloalkyl, (3-6C)cycloalkyl(1-2C)alkyl, phenyl, phenyl(1-4C)alkyl, 4- to 6-membered heterocyclyl, [4- to 6-membered heterocyclyl](1-2C)alkyl, 5- to 10-membered heteroaryl and [5- to 10-membered heteroaryl](1-2C)alkyl, wherein any alkyl, cycloalkyl, cycloalkyl-alkyl, phenyl, phenylalkyl, heterocyclyl, heterocyclyl-alkyl, heteroaryl or heteroaryl-alkyl in Q₂ is optionally substituted with one or more R₃ₑ groups; and
each R₃ₑ group present is independently selected from the group consisting of hydroxy, cyano, oxo, halogen, (1-4C)alkyl, (1-4C)alkoxy, —C(O)—R_{3d}, —C(O)—OR_{3d}, —O—C(O)—R_{3d}, —C(O)—NR_{3d}R₃ₑ, —N(R₃ₑ)C(O)—R_{3d}, —S(O)₀₋₂R_{3d}—, phenyl, 5 or 6-membered heteroaryl, or 4 to 6-membered heterocyclyl,
wherein R_{3d} and R₃ₑ are each independently hydrogen or (1-2C)alkyl; and wherein any alkyl, alkoxy, phenyl, heteroaryl or heterocyclyl group present in a R₃, group, is optionally further substituted by one or more substituents independently selected from hydroxy or halogen
(12) R₃ is hydrogen, cyano, or a group of the formula

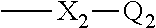

wherein:
X₂ is absent or is selected from the group consisting of —O—, —NR₃ₐ—, —C(O)—O—, —C(O)—N(R₃ₐ)—, —N(R₃ₐ)—C(O)—, —N(R_{3b})—C(O)—NR₃ₐ— or —N(R₃ₐ)SO₂—, where R₃ₐ is selected from the group consisting of hydrogen or methyl;
Q₂ is selected from the group consisting of hydrogen, (1-3C)alkyl, (3-6C)cycloalkyl, (3-6C)cycloalkyl(1-2C)alkyl, phenyl, phenyl(1-4C)alkyl, 4- to 6-membered heterocyclyl, [4- to 6-membered heterocyclyl](1-2C)alkyl, 5- to 10-membered heteroaryl and [5- to 10-membered heteroaryl](1-2C)alkyl, wherein any alkyl, cycloalkyl, cycloalkyl-alkyl, phenyl, phenylalkyl, heterocyclyl, heterocyclyl-alkyl, heteroaryl or heteroaryl-alkyl in Q₂ is optionally substituted with one or more R₃ₑ groups; and
each R_{3c} group present is independently selected from the group consisting of hydroxy, cyano, oxo, halogen, (1-4C)alkyl, (1-4C)alkoxy, —C(O)—R_{3d}, —S(O)₀₋₂R_{3d}—, or 5 or 6-membered heteroaryl;
wherein R_{3d} and R₃ₑ are each independently hydrogen or (1-2C)alkyl; and wherein any alkyl, alkoxy, phenyl, heteroaryl or heterocyclyl group present in a R_{3c} group, is optionally further substituted by one or more substituents independently selected from hydroxy or halogen; (13) R₃ is hydrogen, cyano, or a group of the formula

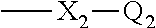

wherein:
   X₂ is absent or is selected from the group consisting of —O—, —NR₃ₐ—, —C(O)—O—,
   —C(O)—N(R₃ₐ)—, —N(R₃ₐ)—C(O)—, —N(R_{3b})—C(O)—NR₃ₐ— or —N(R₃ₐ)SO₂—, where R₃ₐ is selected from the group consisting of hydrogen or methyl;
   Q₂ is selected from the group consisting of hydrogen, (1-3C)alkyl, (3-6C)cycloalkyl, (3-6C)cycloalkyl(1-2C)alkyl, phenyl, phenyl(1-4C)alkyl, 4- to 6-membered heterocyclyl, [4- to 6-membered heterocyclyl](1-2C)alkyl, 5- to 10-membered heteroaryl and [5- to 10-membered heteroaryl](1-2C)alkyl, wherein any alkyl, cycloalkyl, cycloalkyl-alkyl, phenyl, phenylalkyl, heterocyclyl, heterocyclyl-alkyl, heteroaryl or heteroaryl-alkyl in Q₂ is optionally substituted with one or more R₃, groups; and each R_{3c} group present is independently selected from the group consisting of hydroxy, cyano, oxo, halogen, (1-4C)alkyl, (1-4C)alkoxy, —C(O)—R_{3d}, —S(O)₀₋₂R_{3d}—, or 5 or 6-membered heteroaryl;
   wherein R_{3d} and R₃ₑ are each independently hydrogen or methyl;
   and wherein any alkyl, alkoxy, phenyl, heteroaryl or heterocyclyl group present in a R_{3c} group, is optionally further substituted by one or more substituents independently selected from hydroxy or halogen.
(14) R₃ is hydrogen, cyano, or a group of the formula

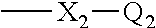

wherein:
X₂ is absent or is selected from the group consisting of —O—, —N(R₃ₐ)—C(O)—, —N(R_{3b})—C(O)—NR₃ₐ— or —N(R₃ₐ)SO₂—, where R₃ₐ is selected from the group consisting of hydrogen or methyl;
Q₂ is selected from the group consisting of hydrogen, (1-3C)alkyl, (3-6C)cycloalkyl, (3-6C)cycloalkyl(1-2C)alkyl, phenyl, phenyl(1-4C)alkyl, 4- to 6-membered heterocyclyl, [4- to 6-membered heterocyclyl](1-2C)alkyl, 5- to 10-membered heteroaryl and [5- to 10-membered heteroaryl](1-2C)alkyl, wherein any alkyl, cycloalkyl, cycloalkyl-alkyl, phenyl, phenylalkyl, heterocyclyl, heterocyclyl-alkyl, heteroaryl or heteroaryl-alkyl in Q₂ is optionally substituted with one or more R_{3c} groups; and
each R₃ₑ group present is independently selected from the group consisting of hydroxy, cyano, oxo, halogen, (1-4C)alkyl, (1-4C)alkoxy, —C(O)—R_{3d} or —S(O)₀. 2R_{3d}—;
wherein R_{3d} and R₃ₑ are each independently hydrogen or methyl;
and wherein any alkyl, alkoxy, phenyl, heteroaryl or heterocyclyl group present in a R_{3c} group, is optionally further substituted by one or more substituents independently selected from hydroxy or halogen.
(15) Q₁ is selected from hydrogen or:
   (i) a group of the formula:

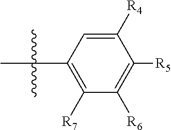

wherein:
R₄ and R₆ are each independently selected from hydrogen, halo, cyano, or a group of the formula:

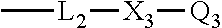

wherein:
L₂ is absent or (1-3C)alkylene;
X₃ is absent or is selected from the group consisting of —O—, —C(O)—, —NR₄ₐ—, —C(O)—O—, —O—C(O)—, —S(O)₀₋₂—, —C(O)—N(R₄ₐ)—, —N(R₄ₐ)—C(O)—, —O—C(O)—N(R₄ₐ)—, —N(R₄ₐ)—C(O)—O—, —N(R_{4b})—C(O)—NR₄ₐ—, —SO₂N(R₄ₐ)— or —N(R₄ₐ)SO₂—, where R₄ₐ and R_{4b} are independently selected from the group consisting of hydrogen or (1-2C)alkyl;
Q₃ is selected from the group consisting of hydrogen, (1-4C)alkyl, (3-6C)cycloalkyl, (3-6C)cycloalkyl(1-2C)alkyl, phenyl, phenyl(1-2C)alkyl, 3 to 8-membered heterocyclyl, [3 to 8-membered heterocyclyl](1-2C)alkyl, 5 or 6-membered heteroaryl and 5 or 6-membered heteroaryl(1-2C)alkyl, wherein any alkyl, cycloalkyl, cycloalkylalkyl, aryl, arylalkyl, heterocyclyl, heterocyclylalkyl, heteroaryl or heteroarylalkyl in Q₃ is optionally substituted with one or more R_{4c} groups; and
each R_{4c} group present is independently selected from the group consisting of hydroxy, cyano, oxo, halogen, (1-4C)alkyl, (1-4C)alkoxy, (3-6C)cycloalkyl, or (3-6C)cycloalkyl(1-2C)alkyl, —NR_{4d}R₄ₑ, —C(O)—R_{4d}, —C(O)—OR_{4d}, —O—C(O)—R_{4d}, —C(O)—NR_{4d}R₄ₑ, —N(R₄ₑ)C(O)—R_{4d}, —S(O)₀₋₂R_{4d}—, —S(O)₂NR_{4d}R₄ₑ, or —N(R₄ₑ)—S(O)₂R_{4d},
wherein R_{4d} and R₄ₑ are each independently hydrogen or (1-4C)alkyl; and wherein any alkyl, alkoxy, cycloalkyl, cycloalkyl-alkyl group present in a R_{4c} group, or any alkyl group present in a R_{4d} or R₄ₑ group, is optionally further substituted by one or more substituents independently selected from hydroxy, cyano, halogen, (1-2C)alkyl, —OR_{4f}, —NR_{4f}R_{4g} and —C(O)—R_{4f}, wherein R₄ᵣ and R_{4g} are both independently selected from hydrogen and (1-2C)alkyl; R₅ is:
a) hydrogen, halo, cyano; or
b) a group of the formula:

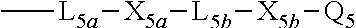

wherein:
L₅ₐ is absent or (1-3C)alkylene;
X₅ₐ is absent or is selected from the group consisting of —O—, —C(O)—, —NR₅ₐ₁—, —C(O)—O—, —O—C(O)—, —S(O)₀₋₂—, —C(O)—N(R₅ₐ₁)—, —N(R₅ₐ₁)—C(O)—, —O—C(O)—N(R₅ₐ), —N(R₅ₐ₁)C(O)—O—, —N(R₅ₐ₂)—C(O)—NR₅ₐ₁—, —SO₂N(R₅ₐ)— or —N(R₅ₐ₁)SO₂—, where R₅ₐ₁ and R₅ₐ₂ are independently selected from the group consisting of hydrogen or (1-4C)alkyl;
L_{5b} is absent or (1-4C)alkylene;
X_{5b} is absent or is selected from the group consisting of —O—, —C(O)—, —NR_{5b1}—, —C(O)—O—, —O—C(O)—, —S(O)₀₋₂—, —C(O)—N(R_{5b})—, —N(R_{5b1})—C(O)—, —O—C(O)—N(R_{5b})—, —N(R_{5b1})—C(O)—O—, —N(R_{5b2})—C(O)—NR_{5b1}—, —SO₂N(R_{5b1})— or —N(R_{5b1})SO₂—, where R_{5b1} and R_{5b2} are independently selected from the group consisting of hydrogen or (1-4C)alkyl;
Q₅ is selected from the group consisting of hydrogen, (1-6C)alkyl, (3-8C)cycloalkyl, (3-8C)cycloalkyl(1-4C)alkyl, phenyl, phenyl(1-2C)alkyl, 3 to 8-membered heterocyclyl, [3 to 8-membered heterocyclyl](1-2C)alkyl, 5 or 6-membered heteroaryl and 5 or 6-membered heteroaryl(1-2C)alkyl and wherein Q₅ is optionally substituted with one or more R_{5c} groups;
and each R_{5c} group present is independently selected from the group consisting of hydroxy, cyano, oxo, halogen, (1-4C)alkyl, (1-4C)alkoxy, (3-6C)cycloalkyl (including spiro-fused (3-6C)cycloalkyl), or (3-6C)cycloalkyl(1-2C)alkyl, —NR_{5d}R₅ₑ, —C(O)—R_{5d}, —C(O)—OR_{5d}, —O—C(O)—R_{5d}, —C(O)—NR_{5d}R₅ₑ, —N(R₅ₑ)C(O)—R_{5d}, —S(O)O-2R_{5d}—, —S(O)₂NR_{5d}R₅ₑ, —N(R₅ₑ)—S(O)₂R_{5d}, phenyl, 5 or 6-membered heteroaryl, or 4 to 8-membered heterocyclyl,
wherein R_{5d} and R₅ₑ are each independently hydrogen, (1-6C)alkyl, (3-6C)cycloalkyl, or (3-6C)cycloalkyl(1-2C)alkyl;
and wherein any alkyl, alkoxy, cycloalkyl, cycloalkyl-alkyl, phenyl, heteroaryl or heterocyclyl group present in a R_{5c} group, or any alkyl, cycloalkyl or cycloalkyl-alkyl group present in a R_{5d} or R₅ₑ group, is optionally further substituted by one or more substituents independently selected from hydroxy, cyano, halo, R_{5f}, —OR_{5f}, —NR_{5f}R_{5g} and —C(O)—R_{5f}, wherein R_{5f} and R_{5g} are both independently selected from hydrogen, (1-4C)alkyl, hydroxy(1-4C)alkyl, halo(1-4C)alkyl, or (1-2C)alkoxy(1-4C)alkyl;
R₇ is selected from hydrogen, halogen, hydroxy, methyl, hydroxymethyl, methoxy, or cyano;
or R₄ and R₅, or R₅ and R₆ are linked to form a fused phenyl, 5- or 6-membered heteroaryl or 5 to 8-membered heterocyclic ring, which is optionally substituted by one or more substituent groups selected from hydroxy, cyano, halo, (1-2C)alkyl, (1-2C)haloalkyl, (1-2C)alkoxy or (1-2C)haloalkoxy;
(ii) a group of the formula:

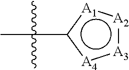

wherein:
A₁ is selected from N, NR_{1N}, O, S or CR₇;
A₂ is selected from N, NR_{1N}, O, S or CR₄;
A₃ is selected from N, NR_{1N}, O, S or CR₅;
A₄ is selected from N, NR_{1N}, O, S or CR₇;
R_{1N} is selected from hydrogen, (1-4C)alkyl, (2-4C)alkanoyl, phenyl, benzyl, (3-6C)cycloalkyl, (3-6C)cycloalkyl-(1-2C)alkyl, a carbon-linked 4 to 7 membered heterocyclyl, or 4 to 7 membered heterocyclyl(1-2C)alkyl, and wherein any alkyl, alkanoyl, cycloalkyl, cycloalkyl-alkyl, phenyl, benzyl, heterocyclyl, heterocyclyl-alkyl group is optionally substituted with one or more R_{5c} groups defined above;
R₄, R₅ and R₇ are as defined above;
and with the proviso that:
   (i) only one of A₁, A₂, A₃ and A₄ can be O or S;
   (ii) only one of A₁, A₂, A₃ and A₄ can be NR_{1N}; and
   (iii) 1 to 4 of A₁, A₂, A₃ and A₄ can be N;
(iii) a group of the formula:

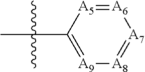

wherein:
A₅ is selected from N or CH;
A₆ is selected from N or CR₄;
A₇ is selected from N or CR₅;
A₈ is selected from N or CR₆;
A₉ is selected from N or CR₇;
R₄, R₅, R₆ and R₇ are as defined above;
and with the proviso that one or two of A₅, A₆, A₇, A₈ and A₉ can be N;
(iv) a group of the formula:

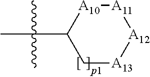

wherein:
p1 is 0 or 1;
A₁₀ is selected from NH, CH₂ or CHR₇;
A₁₁ is selected from NR_{4N}, CH₂ or CHR₄;
A₁₂ is selected from NR_{5N}, CH₂ or CHR₅;
A₁₃ is selected from NR_{5N}, CH₂ or CHR_{O};
R₄, R₅, R₆ and R₇ are as defined above;
R_{4N} and R_{6N} are each independently selected from hydrogen or a group of the formula:

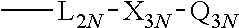

wherein:
L₂N is absent or (1-3C)alkylene;
X_{3N} is absent or is selected from the group consisting of:
   (i) —C(O)—, —S(i)s-2- or —C(O)—N(R₄)— when L₂ is absent; or
   (ii) —O—, —C(O)—, —NR₄ₐ—, —C(O)—O—, —O—C(O)—, —S(O)₀₋₂—, —C(O)—N(R₄ₐ)—, —N(R₄ₐ)—C(O)—, —O—C(O)—N(R₄ₐ)—, —N(R₄)—C(O)—O—, —N(R_{4b})—C(O)—NR₄ₐ—, —SO₂N(R₄ₐ)— or —N(R₄ₐ)SO₂— when L₂ is (1-3C)alkylene;
   wherein R₄ₐ and R_{4b} are as defined above;
Q_{3N} is selected from the group consisting of hydrogen, (1-4C)alkyl, (3-6C)cycloalkyl, (3-6C)cycloalkyl(1-2C)alkyl, phenyl, phenyl(1-4C)alkyl, heterocyclyl, heterocyclyl(1-2C)alkyl, heteroaryl and heteroaryl(1-2C)alkyl, wherein any alkyl, cycloalkyl, cycloalkylalkyl, aryl, arylalkyl, heterocyclyl, heterocyclylalkyl, heteroaryl or heteroarylalkyl in Q₃ is optionally substituted with one or more R₄ₑ groups; and each R₄ₑ group present is as defined above; R_{5N} is:
   a) hydrogen;
   b) a group of the formula:

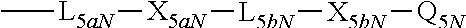

wherein:
L_{5aN} is absent or (1-3C)alkylene;
X_{5aN} is absent or is selected from the group consisting of:
(i) —C(O)—, —S(O)₀₋₂—, —C(O)—N(R₅ₐ₁)— when L_{5aN} is absent; or
(ii) —O—, —C(O)—, —NR₅ₐ₁—, —C(O)—O—, —O—C(O)—, —S(O)₀₋₂—, —C(O)—N(R₅ₐ₁), —N(R₅ₐ₁)C(O)—, —O—C(O)—N(R₅ₐ₁)—, —N(R₅ₐ)C(O)—O—, —N(R₅ₐ₂)—C(O)—NR₅aj-, —SO₂N(R_{5,1})— or —N(R₅ₐ₁)SO₂— when L_{5aN} is present;
where R₅ₐ₁ and R₅ₐ₂ are as defined above;
L_{5bN} is absent or (1-40)alkylene;
X_{5bN} is absent or is selected from the group consisting of —O—, —C(O)—, —NR_{5b1}—, —C(O)—O—, —O—C(O)—, —S(O)₀₋₂—, —C(O)—N(R_{5b})—, —N(R_{5b})—C(O)—, —O—C(O)—N(R_{5b})—, —N(R_{5b1})—C(O)—O—, —N(R_{5b2})—C(O)—NR_{5b}—, —SO₂N(R_{5b1})— or —N(R_{5b1})SO₂—, where R_{5b1} and R_{5b2} are independently selected from the group consisting of hydrogen or (1-4C)alkyl;
Q_{5N} is selected from the group consisting of:
(i) hydrogen;
(ii) (1-6C)alkyl, (3-8C)cycloalkyl, (3-8C)cycloalkyl(1-4C)alkyl, phenyl, phenyl(1-4C)alkyl, 5- or 6-membered heteroaryl, 5- or 6-membered heteroaryl(1-2C)alkyl;
(iii) a carbon-linked 4 to 8 membered heterocyclyl (when -L_{5aN}, X_{5aN}, L_{5bN}, and X_{5bN} are absent) or a 4 to 8 membered heterocyclyl (when one or more of -L_{5aN}, X_{5aN}, L_{5bN}, and X_{5bN} are present), or [4 to 8 membered heterocyclyl](1-2C)alkyl;
and wherein Q_{5N} is optionally substituted with one or more R_{5c} groups as defined above;
or R_{4N} and R_{5N}, or R_{5N} and R_{6N} are linked to form a fused phenyl, 5- or 6-membered heteroaryl or 5 to 8-membered heterocyclic ring, which is optionally substituted by one or more substituent groups selected from hydroxy, cyano, halo, (1-2C)alkyl, (1-2C)haloalkyl, (1-2C)alkoxy or (1-2C)haloalkoxy;
and with the proviso that one or two of A₁₀, A₁₁, A₁₂ and A₁₃ can be N;
(v) a group of the formula:

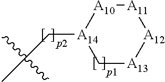

wherein:
p1, A₁₀, A₁₁, A₁₂, and A₁₃ are as defined above;
p2 is 1 to 5;
A₁₄ is C or N;
and with the proviso that one of A₁₀, A₁₁, A₁₂ and A₁₃ can be N.
(16) Q₁ is selected from hydrogen or:
   (i) a group of the formula:

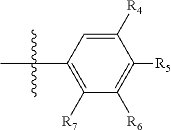

wherein:
R₄ and R₆ are each independently selected from hydrogen, halo, cyano, or a group of the formula:

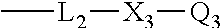

wherein:
L₂ is absent or (1-3C)alkylene;
X₃ is absent or is selected from the group consisting of —O—, —NR₄ₐ—, —C(O)—O—, —O—C(O)—, —S(O)₀₋₂—, —C(O)—N(R₄ₐ)—, —N(R₄ₐ)—C(O)—, —SO₂N(R₄ₐ)— or —N(R₄ₐ)SO₂—, where R₄ₐ and R_{4b} are independently selected from the group consisting of hydrogen or (1-2C)alkyl;
Q₃ is selected from the group consisting of hydrogen, (1-4C)alkyl, (3-6C)cycloalkyl, (3-6C)cycloalkyl(1-2C)alkyl, phenyl, phenyl(1-2C)alkyl, 3 to 8-membered heterocyclyl, [3 to 8-membered heterocyclyl](1-2C)alkyl, 5 or 6-membered heteroaryl and 5 or 6-membered heteroaryl(1-2C)alkyl, wherein any alkyl, cycloalkyl, cycloalkylalkyl, aryl, arylalkyl, heterocyclyl, heterocyclyl-alkyl, heteroaryl or heteroaryl-alkyl in Q₃ is optionally substituted with one or more R_{4c} groups; and each R_{4c} group present is independently selected from the group consisting of hydroxy, cyano, oxo, halogen, (1-4C)alkyl, (1-4C)alkoxy, (3-6C)cycloalkyl, (3-6C)cycloalkyl(1-2C)alkyl, —NR_{4d}R₄ₑ, —C(O)—R_{4d}, —C(O)—OR_{4d}, —O—C(O)—R_{4d}, —C(O)—NR_{4d}R₄ₑ, —N(R₄ₑ)C(O)—R_{4d}, —S(O)₀₋₂R_{4d}—, —S(O)₂NR_{4d}R₄ₑ, or —N(R₄ₑ)—S(O)₂R_{4d},
wherein R_{4d} and R₄ₑ are each independently hydrogen or (1-4C)alkyl; and wherein any alkyl, alkoxy, cycloalkyl, cycloalkyl-alkyl group present in a R₄ₑ group, or any alkyl group present in a R_{4d} or R₄ₑ group, is optionally further substituted by one or more substituents independently selected from hydroxy, cyano, halogen, R_{4f}, —OR_{4f}, —NR_{4f}R_{4g} and —C(O)—R_{4f}, wherein R_{4f} and R_{4g} are both independently selected from hydrogen or (1-2C)alkyl;
R₅ is:
   a) hydrogen, halo, cyano; or
   b) a group of the formula:

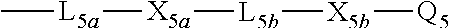

wherein:
L₅ₐ is absent or (1-3C)alkylene;
X₅ₐ is absent or is selected from the group consisting of —O—, —C(O)—, —NR₅ₐ₁—, —C(O)—O—, —O—C(O)—, —S(O)₀₋₂—, —C(O)—N(R₅ₐ₁)—, —N(R₅ₐ₁)—C(O)—, —SO₂N(R₅ₐ₁)— or —N(R₅ₐ₁)SO₂—, where R₅ₐ₁ and R₅ₐ₂ are independently selected from the group consisting of hydrogen or (1-4C)alkyl;
L_{5b} is absent or (1-4C)alkylene;
X_{5b} is absent or is selected from the group consisting of —O—, —C(O)—, —NR_{5b1}—, —C(O)—O—, —O—C(O)—, —S(O)₀₋₂—, —C(O)—N(R_{5b1})—, —N(R_{5b1})—C(O)—, —O—C(O)—N(R_{5b1})—, —N(R_{5b1})—C(O)—O—, —N(R_{5b2})—C(O)—NR_{5b1}—, —SO₂N(R_{5b1})— or —N(R_{5b1})SO₂—, where R_{5b1} and R_{5b2} are independently selected from the group consisting of hydrogen or (1-4C)alkyl;
Q₅ is selected from the group consisting of hydrogen, (1-6C)alkyl, (3-8C)cycloalkyl, (3-8C)cycloalkyl(1-4C)alkyl, phenyl, phenyl(1-2C)alkyl, 3 to 8-membered heterocyclyl, [3 to 8-membered heterocyclyl](1-2C)alkyl, 5 or 6-membered heteroaryl and 5 or 6-membered heteroaryl(1-2C)alkyl and wherein Q₅ is optionally substituted with one or more R_{5c} groups;
and each R₅, group present is independently selected from the group consisting of hydroxy, cyano, oxo, halogen, (1-6C)alkyl, (1-6C)alkoxy, (3-8C)cycloalkyl (including spiro-fused (3-8C)cycloalkyl), or (3-8C)cycloalkyl(1-2C)alkyl, —NR_{5d}R₅ₑ, —C(O)—R_{5d}, —C(O)—OR_{5d}, —O—C(O)—R_{5d}, —C(O)—NR_{5d}R₅ₑ, —N(R₅ₑ)C(O)—R_{5d}, —S(O)₀₋₂R_{5d}—, —S(O)₂NR_{5d}R₅ₑ, —N(R₅ₑ)—S(O)₂R_{5d}, phenyl, 5 or 6-membered heteroaryl, or 4 to 8-membered heterocyclyl,
wherein R_{5d} and R₅ₑ are each independently hydrogen, (1-6C)alkyl, (3-6C)cycloalkyl, or (3-6C)cycloalkyl(1-2C)alkyl;
and wherein any alkyl, alkoxy, cycloalkyl, cycloalkyl-alkyl, phenyl, heteroaryl or heterocyclyl group present in a R_{5c} group, or any alkyl, cycloalkyl or cycloalkyl-alkyl group present in a R_{5d} or R₅ₑ group, is optionally further substituted by one or more substituents independently selected from hydroxy, cyano, halo, R_{5f}, —OR_{5f}, —NR_{5f}R_{5g} and —C(O)—R_{5f}, wherein R_{5f} and R_{5g} are both independently selected from hydrogen, (1-4C)alkyl, hydroxy(1-4C)alkyl, halo(1-4C)alkyl, or (1-2C)alkoxy(1-4C)alkyl;
R₇ is selected from hydrogen, hydroxymethyl, or methoxy;
or R₄ and R₅, or R₅ and R₆ are linked to form a fused phenyl, 5- or 6-membered heteroaryl or 5 to 7-membered heterocyclic ring, which is optionally substituted by one or more substituent groups selected from hydroxy, cyano, halo, (1-2C)alkyl, (1-2C)haloalkyl, (1-2C)alkoxy or (1-2C)haloalkoxy;
(ii) a group of the formula:

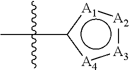

wherein:
A₁ is selected from N, NR_{1N}, O, S or CR₇;
A₂ is selected from N, NR_{1N}, O, S or CR₄;
A₃ is selected from N, NR_{1N}, O, S or CR₅;
A₄ is selected from N, NR_{N}, O, S or CR₇;
R_{1N} is selected from hydrogen, (1-4C)alkyl, (2-4C)alkanoyl, phenyl, benzyl, (3-8C)cycloalkyl, (3-8C)cycloalkyl-(1-2C)alkyl, a carbon-linked 4 to 7 membered heterocyclyl, or 4 to 7 membered heterocyclyl(1-2C)alkyl, and wherein any alkyl, alkanoyl, cycloalkyl, cycloalkyl-alkyl, phenyl, benzyl, heterocyclyl, heterocyclyl-alkyl group is optionally substituted with one or more R_{5c} groups defined above;
R₄, R₅ and R₇ are as defined above;
and with the proviso that:
   (i) only one of A₁, A₂, A₃ and A₄ can be O or S;
   (ii) only one of A₁, A₂, A₃ and A₄ can be NR_{1N};
   (iii) 1 to 3 of A₁, A₂, A₃ and A₄ can be N;
(iii) a group of the formula:

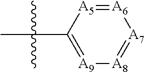

wherein:
A₅ is selected from N or CH;
A₆ is selected from N or CR₄;
A₇ is selected from N or CR₅;
A₈ is selected from N or CR₆;
A₉ is selected from N or CR₇;
R₄, R₅, R₆ and R₇ are as defined above;
and with the proviso that only one of A₅, A₆, A₇, A₈ and A₉ can be N;
(iv) a group of the formula:

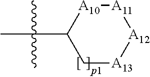

wherein:
p1 is 0 or 1;
A₁₀ is selected from NH, CH₂ or CHR₇;
A₁₁ is selected from NR_{4N}, CH₂ or CHR₄;
A₁₂ is selected from NR_{5N}, CH₂ or CHRs;
A₁₃ is selected from NR_{6N}, CH₂ or CHR₆;
R₄, R₅, R₆ and R₇ are as defined above;
R_{4N} and R_{6N} are each independently selected from hydrogen or a group of the formula:

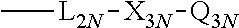

wherein:
L_{2N} is absent or (1-3C)alkylene;
X_{3N} is absent or is selected from the group consisting of:
   (i) —C(O)—, —S(O)₀₋₂— or —C(O)—N(R₄ₐ)— when L₂ is absent; or
   (ii) —O—, —C(O)—, —NR₄ₐ—, —C(O)—O—, —O—C(O)—, —S(O)₀₋₂—, —C(O)—N(R₄ₐ)—, —N(R₄ₐ)—C(O)—, —O—C(O)—N(R₄ₐ)—, —N(R₄ₐ)—C(O)—O—, —N(R_{4b})—C(O)—NR₄ₐ—, —SO₂N(R₄ₐ)— or —N(R₄ₐ)SO₂— when L₂ is (1-3C)alkylene;
   wherein R₄ₐ and R_{4b} are as defined above;
Q_{3N} is selected from the group consisting of hydrogen, (1-6C)alkyl, (3-6C)cycloalkyl, (3-6C)cycloalkyl(1-2C)alkyl, heterocyclyl, heterocyclyl(1-2C)alkyl, heteroaryl and heteroaryl(1-2C)alkyl, wherein any alkyl, cycloalkyl, cycloalkylalkyl, heterocyclyl, heterocyclylalkyl, heteroaryl or heteroarylalkyl in Q₃ is optionally substituted with one or more R₄ₑ groups; and each R_{4c} group present is as defined above; R_{5N} is:
   a) hydrogen;
   b) a group of the formula:

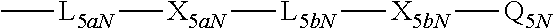

wherein:
L_{5aN} is absent or (1-30)alkylene;
X_{5aN} is absent or is selected from the group consisting of:
(i) —C(O)—, —S(O)₀₋₂—, —C(O)—N(R₅ₐ₁)— when L_{5aN} is absent; or
(ii) —O—, —C(O)—, —NR₅ₐ₁—, —C(O)—O—, —O—C(O)—, —S(O)₀₋₂—, —C(O)—N(R₅ₐ₁)—, —N(R₅ₐ₁)—C(O)—, —O—C(O)—N(R₅ₐ₁)—, —N(R₅ₐ₁)—C(O)—O—, —N(R₅ₐ₂)—C(O)—NR₅ₐ₁—, —SO₂N(R₅ₐ) or —N(R₅ₐ₁)SO₂— when L_{5aN} is present;
where R₅ₐ₁ and R₅ₐ₂ are as defined above;
L_{5bN} is absent or (1-2C)alkylene;
X_{5bN} is absent or is selected from the group consisting of —O—, —C(O)—, —NR_{5b1}—, —C(O)—O—, —O—C(O)—, —S(O)₀₋₂—, —C(O)—N(R_{5b1})—, —N(R_{5b1})—C(O)—, —SO₂N(R_{5b1})— or —N(R_{5b1})SO₂—, where R_{5b}, and R_{5b2} are independently selected from the group consisting of hydrogen or (1-4C)alkyl;
Q_{5N} is selected from the group consisting of:
(i) hydrogen;
(ii) (1-4C)alkyl, (3-6C)cycloalkyl, (3-6C)cycloalkyl(1-1C)alkyl, phenyl, phenyl(1-2C)alkyl, 5- or 6-membered heteroaryl, 5- or 6-membered heteroaryl(1-2C)alkyl;
(iii) a carbon-linked 4 to 8 membered heterocyclyl (when -L_{5aN}, X_{5aN}, L_{5bN}, and X_{5bN} are absent) or a 4 to 8 membered heterocyclyl (when one or more of -L_{5aN}, X_{5aN}, L_{5bN}, and X_{5bN} are present), or [4 to 8 membered heterocyclyl](1-2C)alkyl;
and wherein Q_{5N} is optionally substituted with one or more R₅, groups as defined above;
and with the proviso that one or two of A₁₀, A₁₁, A₁₂ and A₁₃ can be N;
(v) a group of the formula:

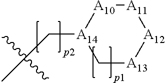

wherein:
p1, A₁₀, A₁₁, A₁₂, and A₁₃ are as defined above;
p2 is 1 to 5; A₁₄ is C or N;
and with the proviso that one of A₁₀, A₁₁, A₁₂ and A₁₃ can be N.
(17) Q₁ is selected from hydrogen or:
   (i) a group of the formula:

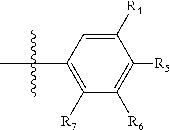

wherein:
R₄ and R₆ are each independently selected from hydrogen, or a group of the formula:

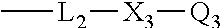

wherein:
L₂ is absent or (1-3C)alkylene;
X₃ is absent or is selected from the group consisting of —O—, —NR₄ₐ—, —S(O)₀₋₂—, —C(O)—N(R₄ₐ)—, —N(R₄ₐ)—C(O)—, —SO₂N(R₄ₐ)— or —N(R₄ₐ)SO₂—, where R₄ₐ and R_{4b} are independently selected from the group consisting of hydrogen or (1-2C)alkyl;
Q₃ is selected from the group consisting of hydrogen, (1-4C)alkyl, (3-6C)cycloalkyl, (3-8C)cycloalkyl(1-4C)alkyl, phenyl, 3 to 8-membered heterocyclyl, 5 or 6-membered heteroaryl or 5 or 6-membered heteroaryl(1-4C)alkyl wherein any alkyl, cycloalkyl, cycloalkylalkyl, phenyl, heterocyclyl, or heteroaryl in Q₃ is optionally substituted with one or more R₄ₑ groups; and
each R_{4c} group present is independently selected from the group consisting of hydroxy, cyano, oxo, halogen, (1-4C)alkyl, (1-4C)alkoxy, (3-6C)cycloalkyl, (3-6C)cycloalkyl(1-2C)alkyl, —NR_{4d}R₄ₑ, —C(O)—R_{4d}, —C(O)—OR_{4d}, —O—C(O)—R_{4d}, —C(O)—NR_{4d}R₄ₑ, —N(R₄ₑ)C(O)—R_{4d}, —S(O)₀₋₂R_{4d}—, —S(O)₂NR_{4d}R₄ₑ, or —N(R₄ₑ)—S(O)₂R_{4d},
wherein R_{4d} and R₄ₑ are each independently hydrogen or (1-4C)alkyl;
R₅ is:
   a) hydrogen, halo, cyano; or
   b) a group of the formula:

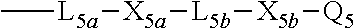

wherein:
L₅ₐ is absent or (1-3C)alkylene;
X₅ₐ is absent or is selected from the group consisting of —O—, —C(O)—, —NR₅ₐ₁—, —S(O)₀₋₂—, —C(O)—N(R₅ₐ₁)—, —N(R₅ₐ₁)—C(O)—, —SO₂N(R₅ₐ₁)— or —N(R₅ₐ₁)SO₂—, where R₅ₐ₁ and R₅ₐ₂ are independently selected from the group consisting of hydrogen or (1-2C)alkyl;
L_{5b} is absent or (1-4C)alkylene;
X_{5b} is absent or is selected from the group consisting of —O—, —C(O)—, —NR_{5b1}—, —S(O)₀₋₂—, —C(O)—N(R_{5b1})—, —N(R_{5b1})—C(O)—, —SO₂N(R_{5b1})— or —N(R_{5b1})SO₂—, where R_{5b1} and R_{5b2} are independently selected from the group consisting of hydrogen or (1-2C)alkyl;
Q₅ is selected from the group consisting of hydrogen, (1-6C)alkyl, (3-8C)cycloalkyl, phenyl, 3 to 8-membered heterocyclyl, or 5 or 6-membered heteroaryl and wherein Q₅ is optionally substituted with one or more R_{5c} groups;
and each R_{5c} group present is independently selected from the group consisting of hydroxy, cyano, halogen, (1-6C)alkyl, (1-6C)alkoxy, (3-8C)cycloalkyl (including spiro-fused (3-8C)cycloalkyl), (3-8C)cycloalkyl(1-2C)alkyl, —NR_{5d}R₅ₑ, —C(O)—Rd, —C(O)—NR_{5d}R₅ₑ, —N(R₅ₑ)C(O)—R_{5d}, —S(O)₀₋₂R_{5d}—, —S(O)₂NR_{5d}R₅ₑ, —N(R₅ₑ)—S(O)₂R_{5d}, phenyl, 5 or 6-membered heteroaryl, or 4 to 8-membered heterocyclyl,
wherein R_{5d} and R₅ₑ are each independently hydrogen, (1-6C)alkyl, (3-6C)cycloalkyl, or (3-6C)cycloalkyl(1-2C)alkyl;
and wherein any alkyl, alkoxy, cycloalkyl, cycloalkyl-alkyl, phenyl, heteroaryl or heterocyclyl group present in a R₅, group, or any alkyl, cycloalkyl or cycloalkyl-alkyl group present in a R_{5d} or R₅ₑ group, is optionally further substituted by one or more substituents independently selected from hydroxy, cyano, halo, R_{5f}, —OR_{5f}, —NR_{5f}R_{5g} and —C(O)—R_{5f}, wherein R_{5f} and R_{5g} are both independently selected from hydrogen, (1-4C)alkyl, hydroxy(1-4C)alkyl, halo(1-4C)alkyl, or (1-2C)alkoxy(1-4C)alkyl;
R₁ is selected from hydrogen, hydroxymethyl, or methoxy;
or R₄ and R₅, or R₅ and R₆ are linked to form a fused phenyl, 5- or 6-membered heteroaryl or 5 to 7-membered heterocyclic ring, which is optionally substituted by one or more substituent groups selected from hydroxy, cyano, halo, (1-2C)alkyl, (1-2C)haloalkyl, (1-2C)alkoxy or (1-2C)haloalkoxy;
(ii) a group of the formula:

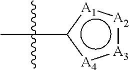

wherein:
A₁ is selected from N, NR_{1N}, O, S or CR₇;
A₂ is selected from N, NR_{1N}, O, S or CR₄;
A₃ is selected from N, NR_{1N}, O, S or CR₅;
A₄ is selected from N, NR_{1N}, O, S or CR₇;
R_{1N} is selected from hydrogen, (1-4C)alkyl, (2-4C)alkanoyl, phenyl, benzyl, (3-8C)cycloalkyl, (3-8C)cycloalkyl-(1-2C)alkyl, a carbon-linked 4 to 7 membered heterocyclyl, or 4 to 7 membered heterocyclyl(1-2C)alkyl, and wherein any alkyl, alkanoyl, cycloalkyl, cycloalkyl-alkyl, phenyl, benzyl, heterocyclyl, heterocyclyl-alkyl group is optionally substituted with one or more R_{5c} groups defined above;
R₄, R₅ and R₇ are as defined above;
and with the proviso that:
   (i) only one of A₁, A₂, A₃ and A₄ can be O or S;
   (ii) only one of A₁, A₂, A₃ and A₄ can be NR_{1N};
   (iii) 1 to 3 of A₁, A₂, A₃ and A₄ can be N;
(iii) a group of the formula:

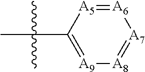

wherein:
A₅ is selected from N or CH;
A₆ is selected from N or CR₄;
A₇ is selected from N or CR₅;
A₈ is selected from N or CR₅;
A₉ is selected from N or CR₇;
R₄, R₅, R₆ and R₇ are as defined above;
and with the proviso that only one of A₅, A₆, A₇, A₈ and A₉ can be N;
(iv) a group of the formula:

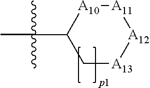

wherein:
p1 is 0 or 1;
A₁₀ is selected from NH, CH₂ or CHR₇;
A₁₁ is selected from NR_{4N}, CH₂ or CHR₄;
A₁₂ is selected from NR_{5N}, CH₂ or CHR₅;
A₁₃ is selected from NR_{6N}, CH₂ or CHR_{O};
R₄, R₅, R₆ and R₇ are as defined above;
R_{4N} and R_{6N} are each independently selected from hydrogen or a group of the formula:

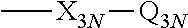

wherein:
X_{3N} is absent or is selected from the group consisting of: —C(O)—, —S(O)₀₋₂— or —C(O)—N(R₄ₐ)—;
   wherein R₄ₐ is as defined above;
Q_{3N} is selected from the group consisting of hydrogen, (1-4C)alkyl, (3-6C)cycloalkyl, (3-6C)cycloalkyl(1-2C)alkyl, heterocyclyl, heteroaryl and, wherein any alkyl, cycloalkyl, cycloalkylalkyl, heterocyclyl or heteroaryl in Q₃ is optionally substituted with one or more R₄ₑ groups; and each R₄ₑ group present is as defined above; R_{5N} is:
   a) hydrogen;
   b) a group of the formula:

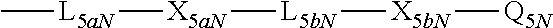

wherein:
L_{5aN} is absent or (1-30)alkylene;
X_{5aN} is absent or is selected from the group consisting of:
(i) —C(O)—, —S(O)₀₋₂—, —C(O)—N(R₅ₐ₁)— when L_{5aN} is absent; or
(ii) —O—, —C(O)—, —NR₅ₐ₁—, —C(O)—O—, —O—C(O)—, —S(O)—2—, —C(O)—N(R₅ₐ₁)—, —N(R₅ₐ₁)—C(O)—, —O—C(O)—N(R₅ₐ₁)—, —N(R₅ₐ₁)—C(O)—O—, —N(R₅ₐ₂)—C(O)—NR₅ₐ₁—, —SO₂N(R₅ₐ₁)— or —N(R₅ₐ₁)SO₂— when L_{5aN} is present;
where R₅ₐ₁ and R₅ₐ₂ are as defined above;
L_{5bN} is absent or (1-2C)alkylene;
X_{5bN} is absent or is selected from the group consisting of —O—, —C(O)—, —NR_{5b1}—, —S(O)₀₋₂—, —C(O)—N(R_{5b1})—, —N(R_{5b1})—C(O)—, —SO₂N(R_{5b1})— or —N(R_{5b1})SO₂—, where R_{5b1} and R_{5b2} are independently selected from the group consisting of hydrogen or (1-2C)alkyl;
Q_{5N} is selected from the group consisting of:
(i) hydrogen;
(ii) (1-4C)alkyl, (3-6C)cycloalkyl, (3-6C)cycloalkyl(1-1C)alkyl, phenyl, phenyl(1-2C)alkyl, 5- or 6-membered heteroaryl, 5- or 6-membered heteroaryl(1-2C)alkyl;
and wherein Q_{5N} is optionally substituted with one or more R_{5c} groups as defined above;
and with the proviso that one or two of A₁₀, A₁₁, A₁₂ and A₁₃ can be N;
(v) a group of the formula:

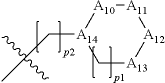

wherein:
p1, A₁₀, A₁₁, A₁₂, and A₁₃ are as defined above;
p2 is 1 to 4;
A₁₄ is C or N;
and with the proviso that one of A₁₀, A₁₁, A₁₂ and A₁₃ can be N.
(18) Q₁ is selected from hydrogen or:
   (i) a group of the formula:

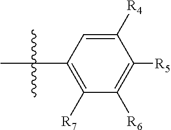

wherein:
R₄ and R₆ are each independently selected from hydrogen, or a group of the formula:

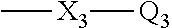

wherein:
X₃ is absent or is selected from the group consisting of —O—, —NR₄ₐ—, —C(O)—N(R₄ₐ)—, —N(R₄ₐ)—C(O)—, —SO₂N(R₄ₐ)— or —N(R₄ₐ)SO₂—, where R₄ₐ and R_{4b} are independently selected from the group consisting of hydrogen or (1-2C)alkyl;
Q₃ is selected from the group consisting of hydrogen, (1-4C)alkyl, (3-6C)cycloalkyl, (3-6C)cycloalkyl(1-4C)alkyl, phenyl, 3 to 8-membered heterocyclyl, 5 or 6-membered heteroaryl or 5 or 6-membered heteroaryl(1-4C)alkyl wherein any alkyl, cycloalkyl, cycloalkylalkyl, phenyl, heterocyclyl or heteroaryl in Q₃ is optionally substituted with one or more R₄ₑ groups; and
each R_{4c} group present is independently selected from the group consisting of hydroxy, cyano, oxo, halogen, (1-4C)alkyl, (1-4C)alkoxy, (3-6C)cycloalkyl, (3-6C)cycloalkyl(1-2C)alkyl, —NR_{4d}R₄ₑ, —C(O)—R_{4d}, —C(O)—OR_{4d}, —O—C(O)—R_{4d}, —C(O)—NR_{4d}R₄ₑ, —N(R₄ₑ)C(O)—R_{4d}, —S(O)₀₋₂R_{4d}—, —S(O)₂NR_{4d}R₄ₑ, or —N(R₄ₑ)—S(O)₂R_{4d},
wherein R_{4d} and R₄ₑ are each independently hydrogen or (1-2C)alkyl;
R₅ is:
   a) hydrogen, halo, cyano; or
   b) a group of the formula:

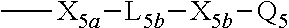

wherein:
X₅ₐ is absent or is selected from the group consisting of —O—, —C(O)—, —NR₅ₐ₁—, —S(O)₀₋₂—, —C(O)—N(R₅ₐ₁)—, —N(R₅ₐ₁)—C(O)—, —SO₂N(R₅ₐ₁)— or —N(R₅ₐ₁)SO₂—, where R₅ₐ₁ and R₅ₐ₂ are independently selected from the group consisting of hydrogen or (1-2C)alkyl;
L_{5b} is absent or (1-4C)alkylene;
X_{5b} is absent or is selected from the group consisting of —O—, —C(O)—, —NR_{5b1}—, —S(O)₀₋₂—, —C(O)—N(R_{5b1})—, —N(R_{5b1})—C(O)—, —SO₂N(R_{5b1})— or —N(R_{5b1})SO₂—, where R_{5b1} and R_{5b2} are independently selected from the group consisting of hydrogen or (1-2C)alkyl;
Q₅ is selected from the group consisting of hydrogen, (1-6C)alkyl, (3-8C)cycloalkyl, phenyl, 3 to 8-membered heterocyclyl, or 5 or 6-membered heteroaryl and wherein Q₅ is optionally substituted with one or more R_{5c} groups;
and each R₅, group present is independently selected from the group consisting of hydroxy, cyano, halogen, (1-6C)alkyl, (1-6C)alkoxy, (3-8C)cycloalkyl (including spiro-fused (3-8C)cycloalkyl), (3-8C)cycloalkyl(1-2C)alkyl, —NR_{5d}R₅ₑ, —C(O)—R_{5d}, —C(O)—NR_{5d}R₅ₑ, —N(R₅ₑ)C(O)—R_{5d}, —S(O)₀₋₂R_{5d}—, —S(O)₂NR_{5d}R₅ₑ, —N(R₅ₑ)—S(O)₂R_{5d}, phenyl, 5 or 6-membered heteroaryl, or 4 to 8-membered heterocyclyl,
wherein R_{5d} and R₅ₑ are each independently hydrogen, (1-6C)alkyl, (3-6C)cycloalkyl, or (3-6C)cycloalkyl(1-2C)alkyl;
and wherein any alkyl, alkoxy, cycloalkyl, cycloalkyl-alkyl, phenyl, heteroaryl or heterocyclyl group present in a R_{5c} group, or any alkyl, cycloalkyl or cycloalkyl-alkyl group present in a R_{5d} or R₅ₑ group, is optionally further substituted by one or more substituents independently selected from hydroxy, cyano, halo, R_{5f}, —OR_{5f}, —NR_{5f}R_{5g} and —C(O)—R_{5f}, wherein R_{5f} and R_{5g} are both independently selected from hydrogen, (1-4C)alkyl, hydroxy(1-4C)alkyl, halo(1-4C)alkyl, or (1-2C)alkoxy(1-4C)alkyl; R₇ is selected from hydrogen, hydroxymethyl, or methoxy;
or R₄ and R₅, or R₅ and R₆ are linked to form a fused phenyl, 5- or 6-membered heteroaryl or 5 to 7-membered heterocyclic ring, which is optionally substituted by one or more substituent groups selected from hydroxy, cyano, halo, (1-2C)alkyl, (1-2C)haloalkyl, (1-2C)alkoxy or (1-2C)haloalkoxy;
(ii) a group of the formula:

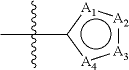

wherein:
A₁ is selected from N, NR_{1N}, O, S or CR₇;
A₂ is selected from N, NR_{1N}, O, S or CR₄;
A₃ is selected from N, NR_{1N}, O, S or CR₅;
A₄ is selected from N, NR_{1N}, O, S or CR₇;
R_{1N} is selected from hydrogen, (1-4C)alkyl, (2-4C)alkanoyl, (3-6C)cycloalkyl, (3-6C)cycloalkyl-(1-2C)alkyl, a carbon-linked 4 to 7 membered heterocyclyl, or 4 to 7 membered heterocyclyl(1-2C)alkyl, and wherein any alkyl, alkanoyl, cycloalkyl, cycloalkyl-alkyl, heterocyclyl, heterocyclyl-alkyl group is optionally substituted with one or more R_{5c} groups defined above;
R₄, R₅ and R₇ are as defined above;
and with the proviso that:
   (i) only one of A₁, A₂, A₃ and A₄ can be O or S;
   (ii) only one of A₁, A₂, A₃ and A₄ can be NR_{1N};
   (iii) 1 to 3 of A₁, A₂, A₃ and A₄ can be N;
(iii) a group of the formula:

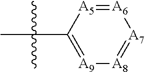

wherein:
A₅ is selected from N or CH;
A₆ is selected from N or CR₄;
A₇ is selected from N or CR₅;
A₉ is selected from N or CR₆;
A₉ is selected from N or CR₇;
R₄, R₅, R₆ and R₇ are as defined above;
and with the proviso that only one of A₅, A₈, A₇, A₈ and A₉ can be N;
(iv) a group of the formula:

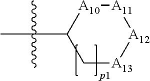

wherein:
p1 is 0 or 1;
A₁₀ is selected from CH₂ or CHR₇;
A₁₁ is selected from CH₂ or CHR₄;
A₁₂ is selected from NR_{5N}, CH₂ or CHRs;
A₁₃ is selected from CH₂ or CHRs;
R₄, R₅, R₆ and R₇ are as defined above;
R_{5N} is:
   a) hydrogen;
   b) a group of the formula:

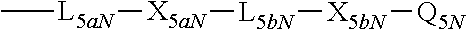

wherein:
L_{5aN} is absent or (1-3C)alkylene;
X_{5aN} is absent or is selected from the group consisting of:
(i) —C(O)—, —S(O)₀₋₂—, —C(O)—N(R₅ₐ₁)— when L_{5aN} is absent; or
(ii) —O—, —C(O)—, —NR₅ₐ₁—, —C(O)—O—, —O—C(O)—, —S(O)₀₋₂—, —C(O)—N(R₅ₐ₁)—, —N(R₅ₐ₁)—C(O)—, —O—C(O)—N(R₅ₐ₁)—, —N(R₅ₐ₁)—C(O)—O—, —N(R₅ₐ₂)—C(O)—NR₅ₐ₁—, —SO₂N(R₅ₐ₁)— or —N(R₅ₐ₁)SO₂— when L_{5aN} is present;
where R₅ₐ₁ and R₅ₐ₂ are as defined above;
L_{5bN} is absent or (1-20)alkylene;
X_{5bN} is absent or is selected from the group consisting of —O—, —C(O)—, —NR_{5b1}—, —S(O)₀₋₂—, —C(O)—N(R_{5b1})—, —N(R_{5b1})—C(O)—, —SO₂N(R_{5b1})— or —N(R_{5b1})SO₂—, where R_{5b1} and R_{5b2} are independently selected from the group consisting of hydrogen or (1-2C)alkyl;
Q_{5N} is selected from the group consisting of:
(i) hydrogen;
(ii) (1-4C)alkyl, (3-6C)cycloalkyl, (3-6C)cycloalkyl(1-1C)alkyl, phenyl, phenyl(1-2C)alkyl, 5- or 6-membered heteroaryl, 5- or 6-membered heteroaryl(1-2C)alkyl;
and wherein Q_{5N} is optionally substituted with one or more R_{5c} groups as defined above;
and with the proviso that one or two of A₁₀, A₁₁, A₁₂ and A₁₃ can be N;
(v) a group of the formula:

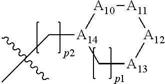

wherein:
p1, A₁₀, A₁₁, A₁₂, and A₁₃ are as defined above;
p2 is 1 to 4;
A₁₄ is C or N;
and with the proviso that one of A₁₀, A₁₁, A₁₂ and A₁₃ can be N.
(19) Q₁ is selected from hydrogen or:
   (i) a group of the formula:

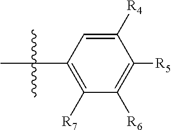

wherein:
R₄ and R₆ are each independently selected from hydrogen, or a group of the formula:

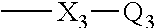

wherein:
X₃ is absent or is selected from the group consisting of —O—, —NR₄ₐ—, —C(O)—N(R₄ₐ)—, —N(R₄ₐ)—C(O)—, —SO₂N(R₄ₐ)— or —N(R₄ₐ)SO₂—, where R₄ₐ and R_{4b} are independently selected from the group consisting of hydrogen or methyl;
Q₃ is selected from the group consisting of hydrogen, (1-4C)alkyl, (3-6C)cycloalkyl, (3-6C)cycloalkyl(1-4C)alkyl, phenyl, 3 to 8-membered heterocyclyl, 5 or 6-membered heteroaryl or 5 or 6-membered heteroaryl(1-4C)alkyl wherein any alkyl, cycloalkyl, cycloalkylalkyl, phenyl, heterocyclyl or heteroaryl in Q₃ is optionally substituted with one or more R₄ₑ groups; and
each R_{4c} group present is independently selected from the group consisting of hydroxy, cyano, oxo, halogen, methyl, methoxy, (3-6C)cycloalkyl, —NR_{4d}R₄ₑ, —C(O)—R_{4d} or —C(O)—OR_{4d}, wherein R_{4d} and R₄ₑ are each independently hydrogen or methyl;
R₅ is:
   a) hydrogen, halo, cyano; or
   b) a group of the formula:

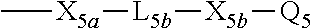

wherein:
X₅ₐ is absent or is selected from the group consisting of —O—, —C(O)—, —NR₅ₐ₁— or —N(R₅ₐ₁)—C(O)—, where R₅ₐ₁ is independently selected from the group consisting of hydrogen or methyl;
L_{5b} is absent or (1-4C)alkylene;
X_{5b} is absent or is selected from the group consisting of —O—, —C(O)— or —NR_{5b1}—, where R_{5b}, and R_{5b2} are independently selected from the group consisting of hydrogen or (1-2C)alkyl;
Q₅ is selected from the group consisting of hydrogen, (1-6C)alkyl or 3 to 8-membered heterocyclyl and wherein Q₅ is optionally substituted with one or more R_{5c} groups;
and each R₅, group present is independently selected from the group consisting of hydroxy, cyano, halogen, methyl, methoxy
R₇ is selected from hydrogen or methoxy;
(ii) a group of the formula:

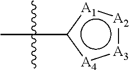

wherein:
A₁ is selected from N, NR_{1N}, O, S or CR₇;
A₂ is selected from N, NR_{1N}, O, S or CR₄;
A₃ is selected from N, NR_{1N}, O, S or CR₅;
A₄ is selected from N, NR_{1N}, O, S or CR₇;
R_{1N} is selected from hydrogen, (1-4C)alkyl, (2-4C)alkanoyl, (3-6C)cycloalkyl, (3-6C)cycloalkyl-(1-2C)alkyl, a carbon-linked 4 to 7 membered heterocyclyl, or 4 to 7 membered heterocyclyl(1-2C)alkyl, and wherein any alkyl, alkanoyl, cycloalkyl, cycloalkyl-alkyl, heterocyclyl, heterocyclyl-alkyl group is optionally substituted with one or more R₅, groups defined above; R₄, R₅ and R₇ are as defined above;
and with the proviso that:
   (i) only one of A₁, A₂, A₃ and A₄ can be O or S;
   (ii) only one of A₁, A₂, A₃ and A₄ can be NR_{1N};
   (iii) 1 to 3 of A₁, A₂, A₃ and A₄ can be N;
(iii) a group of the formula:

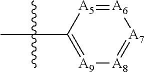

wherein:
A₅ is selected from N or CH;
A₉ is selected from N or CR₄;
A₇ is selected from N or CR₅;
A₉ is selected from N or CR₆;
A₉ is selected from N or CR₇;
R₄, R₅, R₆ and R₇ are as defined above;
and with the proviso that only one of A₅, A₆, A₇, A₈ and A₉ can be N;
(iv) a group of the formula:

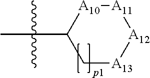

wherein:
p1 is 0 or 1;
A₁₀ is selected from CH₂ or CHR₇;
A₁₁ is selected from CH₂ or CHR₄;
A₁₂ is selected from NR_{5N}, CH₂ or CHR₅;
A₁₃ is selected from CH₂ or CHR₆;
R₄, R₅, R₆ and R₇ are as defined above;
R_{5N} is:
   a) hydrogen;
   b) a group of the formula:

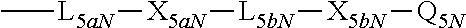

wherein:
L_{5aN} is absent or (1-30)alkylene;
X_{5aN} is absent or is selected from the group consisting of:
(i) —C(O)—, —S(O)₀₋₂— or —C(O)—N(R₅ₐ₁)— when L_{5aN} iS absent; or
(ii) —O—, —C(O)—, —NR₅ₐ₁—, —C(O)—O—, —O—C(O)—, —S(O)O-2—, —C(O)—N(R₅ₐ₁)—, —N(R₅ₐ₁)—C(O)—, —O—C(O)—N(R₅ₐ₁)—, —N(R₅ₐ₁)—C(O)—O— or —N(R₅ₐ₂)—C(O)—NR₅ₐ₁— when L_{5aN} is present;
where R₅ₐ₁ and R₅ₐ₂ are as defined above;
L_{5bN} is absent or (1-2C)alkylene;
X_{5bN} is absent or is selected from the group consisting of —O—, —C(O)—, —NR_{5b1}—, —S(O)₀₋₂—, —C(O)—N(R_{5b1})— or —N(R_{5b1})—C(O)—,
where R_{5b1} and R_{5b2} are independently selected from the group consisting of hydrogen or (1-2C)alkyl;
Q_{5N} is selected from the group consisting of:
(i) hydrogen;
(ii) (1-4C)alkyl or (3-6C)cycloalkyl;
and wherein Q_{5N} is optionally substituted with one or more R_{5c} groups as defined above;
and with the proviso that one or two of A₁₀, A₁₁, A₁₂ and A₁₃ can be N;
(v) a group of the formula:

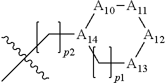

wherein:
p1, A₁₀, A₁₁, A₁₂, and A₁₃ are as defined above;
p2 is 1 to 4;
A₁₄ is C or N;
and with the proviso that one of A₁₀, A₁₁, A₁₂ and A₁₃ can be N.
(20) Q₁ is selected from hydrogen or:
   (i) a group of the formula:

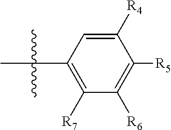

wherein:
one of R₄ and R₆ is hydrogen, and the other is hydrogen or a group of the formula:

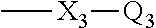

wherein:
X₃ is absent or is selected from the group consisting of —O—, —NR₄ₐ—, —C(O)—N(R₄ₐ)—, —N(R₄ₐ)—C(O)—, —SO₂N(R₄ₐ)— or —N(R₄ₐ)SO₂—, where R₄ₐ and R_{4b} are independently selected from the group consisting of hydrogen or methyl;
Q₃ is selected from the group consisting of hydrogen, (1-4C)alkyl, (3-6C)cycloalkyl, (3-6C)cycloalkyl(1-4C)alkyl, phenyl, 3 to 8-membered heterocyclyl, 5 or 6-membered heteroaryl or 5 or 6-membered heteroaryl(1-4C)alkyl wherein any alkyl, cycloalkyl, cycloalkylalkyl, phenyl, heterocyclyl or heteroaryl in Q₃ is optionally substituted with one or more R₄ₑ groups; and
each R_{4c} group present is independently selected from the group consisting of hydroxy, cyano, oxo, halogen, methyl, methoxy, (3-6C)cycloalkyl, —NR_{4d}R₄ₑ, —C(O)—R_{4d} or —C(O)—OR_{4d},
wherein R_{4d} and R₄ₑ are each independently hydrogen or methyl;
R₅ is hydrogen or a group of the formula:

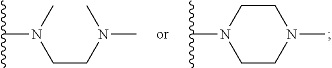

R₁ is selected from hydrogen or methoxy;
(ii) a group of the formula:

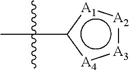

wherein:
A₁ is selected from N, NR_{1N}, O, S or CR₇;
A₂ is selected from N, NR_{1N}, O, S or CR₄;
A₃ is selected from N, NR_{1N}, O, S or CR₅;
A₄ is selected from N, NR_{1N}, O, S or CR₇;
R_{1N} is selected from hydrogen, (1-4C)alkyl, (2-4C)alkanoyl, (3-6C)cycloalkyl, (3-6C)cycloalkyl-(1-2C)alkyl, a carbon-linked 4 to 7 membered heterocyclyl, or 4 to 7 membered heterocyclyl(1-2C)alkyl, and wherein any alkyl, alkanoyl, cycloalkyl, cycloalkyl-alkyl, heterocyclyl, heterocyclyl-alkyl group is optionally substituted with one or more R_{5c} groups defined above;
R₄, R₅ and R₇ are as defined above;
and with the proviso that:
   (i) only one of A₁, A₂, A₃ and A₄ can be O or S;
   (ii) only one of A₁, A₂, A₃ and A₄ can be NR_{1N};
   (iii) 1 to 3 of A₁, A₂, A₃ and A₄ can be N;
(iii) a group of the formula:

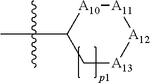

wherein:
p1 is 0 or 1;
A₁₀ is selected from CH₂ or CHR₇;
A₁₁ is selected from CH₂ or CHR₄;
A₁₂ is selected from NR_{5N}, CH₂ or CHR₅;
A₁₃ is selected from CH₂ or CHR₆;
R₄, R₅, R₆ and R₇ are as defined above;
R_{5N} is:
   a) hydrogen;
   b) a group of the formula:

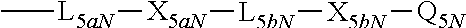

wherein:
L_{5aN} is absent or (1-30)alkylene;
X_{5aN} is absent or is selected from the group consisting of:
(i) —C(O)—, —S(O)₀₋₂— or —C(O)—N(R₅ₐ₁)— when L_{5aN} is absent; or
(ii) —O—, —C(O)—, —NR₅ₐ₁—, —C(O)—O—, —O—C(O)—, —S(O)₀₋₂—, —C(O)—N(R₅ₐ₁)—, —N(R₅ₐ₁)—C(O)—, —O—C(O)—N(R₅ₐ₁)—, —N(R₅ₐ₁)—C(O)—O— or —N(R₅ₐ₂)—C(O)—NR₅ₐ₁— when L_{5aN} is present;
where R₅ₐ₁ and R₅ₐ₂ are as defined above;
L_{5bN} is absent or (1-20)alkylene;
X_{5bN} is absent or is selected from the group consisting of —O—, —C(O)—, —NR_{5b1}—, —S(O)₀₋₂—, —C(O)—N(R_{5b1})— or —N(R_{5b1})—C(O)—, where R_{5b}, and R_{5b2} are independently selected from the group consisting of hydrogen or (1-2C)alkyl;
Q_{5N} is selected from the group consisting of:
(i) hydrogen;
(ii) (1-4C)alkyl or (3-6C)cycloalkyl;
and wherein Q_{5N} is optionally substituted with one or more R_{5c} groups as defined above;
and with the proviso that one or two of A₁₀, A₁₁, A₁₂ and A₁₃ can be N;
(iv) a group of the formula:

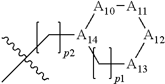

wherein:
p1, A₁₀, A₁₁, A₁₂, and A₁₃ are as defined above;
p2 is 1 to 4;
A₁₄ is C or N;
and with the proviso that one of A₁₀, A₁₁, A₁₂ and A₁₃ can be N.
(21) R₃ is halo, cyano, or a group of the formula

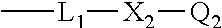

wherein:
L₁ is absent or (1-2C)alkylene;
X₂ is absent or is selected from the group consisting of —O—, —C(O)—, —NR₃ₐ—, —C(O)—O—, —O—C(O)—, —S(O)₀₋₂—, —C(O)—N(R₃ₐ)—, —N(R₃ₐ)—C(O)—, —O—C(O)—N(R₃ₐ)—, —N(R₃ₐ)—C(O)—O—, —N(R_{3b})—C(O)—NR₃ₐ—, —SO₂N(R₃ₐ)—, or —N(R₃ₐ)SO₂—, where R₃ₐ and R_{3b} are independently selected from the group consisting of hydrogen or (1-2C)alkyl;
Q₂ is selected from the group consisting of hydrogen, (1-4C)alkyl, (3-6C)cycloalkyl, (3-6C)cycloalkyl(1-2C)alkyl, phenyl, phenyl(1-4C)alkyl, 3- to 10-membered heterocyclyl, [3- to 10-membered heterocyclyl](1-2C)alkyl, 5- to 10-membered heteroaryl and [5- to 10-membered heteroaryl](1-2C)alkyl, wherein any alkyl, cycloalkyl, cycloalkyl-alkyl, phenyl, phenylalkyl, heterocyclyl, heterocyclyl-alkyl, heteroaryl or heteroaryl-alkyl in Q₂ is optionally substituted with one or more R₃, groups; and each R₃, group present is independently selected from the group consisting of hydroxy, cyano, oxo, halogen, (1-4C)alkyl, (1-4C)alkoxy, (3-6C)cycloalkyl, or (3-6C)cycloalkyl(1-2C)alkyl, —NR_{3d}R₃ₑ, —C(O)—R_{3d}, —C(O)—OR_{3d}, —O—C(O)—R_{3d}, —C(O)—NR_{3d}R₃ₑ, —N(R₃ₑ)C(O)—R_{3d}, —S(O)₀₋₂R_{3d}—, —S(O)₂NR_{3d}R₃ₑ, N(R₃ₑ)—S(O)₂R_{3d}, phenyl, 5 or 6-membered heteroaryl, or 4 to 6-membered heterocyclyl, wherein R_{3d} and R₃ₑ are each independently hydrogen, (1-4C)alkyl, (3-6C)cycloalkyl, or (3-6C)cycloalkyl(1-2C)alkyl;
and wherein any alkyl, alkoxy, cycloalkyl, cycloalkyl-alkyl, phenyl, heteroaryl or heterocyclyl group present in a R₃ₑ group, or any alkyl, cycloalkyl, or cycloalkyl-alkyl group present in a R_{3d} or R₃ₑ group, is optionally further substituted by one or more substituents independently selected from hydroxy, cyano, halogen, (1-2C)alkyl, —OR_{3f}, —NR_{3f}R_{3g} and —C(O)—R_{3f}, wherein R_{3f} and R_{3g} are both independently selected from hydrogen and (1-2C)alkyl;
(22) R₃ is halo, cyano, or a group of the formula

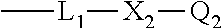

wherein:
L₁ is absent or (1-2C)alkylene;
X₂ is absent or is selected from the group consisting of —O—, —C(O)—, —NR₃ₐ—, —C(O)—O—, —O—C(O)—, —S(O)₀₋₂—, —C(O)—N(R₃ₐ)—, —N(R₃ₐ)—C(O)—, —N(R_{3b})—C(O)—O—, —N(R_{3b})—C(O)—NR₃ₐ—, —SO₂N(R₃ₐ)— or —N(R₃ₐ)SO₂—, where R₃ₐ is selected from the group consisting of hydrogen or (1-2C)alkyl;
Q₂ is selected from the group consisting of hydrogen, (1-4C)alkyl, (3-6C)cycloalkyl, (3-6C)cycloalkyl(1-2C)alkyl, phenyl, phenyl(1-4C)alkyl, 3- to 10-membered heterocyclyl, [3- to 10-membered heterocyclyl](1-2C)alkyl, 5- to 10-membered heteroaryl and [5- to 10-membered heteroaryl](1-2C)alkyl, wherein any alkyl, cycloalkyl, cycloalkyl-alkyl, phenyl, phenylalkyl, heterocyclyl, heterocyclyl-alkyl, heteroaryl or heteroaryl-alkyl in Q₂ is optionally substituted with one or more R₃ₑ groups; and
each R₃ₑ group present is independently selected from the group consisting of hydroxy, cyano, oxo, halogen, (1-4C)alkyl, (1-4C)alkoxy, (3-6C)cycloalkyl, or (3-6C)cycloalkyl(1-2C)alkyl, —NR_{3d}R₃ₑ, —C(O)—R_{3d}, —C(O)—OR_{3d}, —O—C(O)—R_{3d}, —C(O)—NR_{3d}R₃ₑ, —N(R₃ₑ)C(O)—R_{3d}, —S(O)₀₋₂R_{3d}—, —S(O)₂NR_{3d}R₃ₑ, —N(R₃ₑ)—S(O)₂R_{3d}, phenyl, 5 or 6-membered heteroaryl, or 4 to 6-membered heterocyclyl,
wherein R_{3d} and R₃ₑ are each independently hydrogen or (1-2C)alkyl; and wherein any alkyl, alkoxy, cycloalkyl, cycloalkyl-alkyl, phenyl, heteroaryl or heterocyclyl group present in a R₃ₑ group, or any alkyl group present in a R_{3d} or R₃ₑ group, is optionally further substituted by one or more substituents independently selected from hydroxy, cyano, halogen, (1-2C)alkyl, or —OR_{3f}, wherein R_{3f} is selected from hydrogen and (1-2C)alkyl.
(23) R₃ is cyano, or a group of the formula

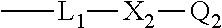

wherein:
L₁ is absent or (1-2C)alkylene;
X₂ is absent or is selected from the group consisting of —O—, —C(O)—, —NR₃ₐ—, —C(O)—O—, —O—C(O)—, —S(O)₀₋₂—, —C(O)—N(R₃ₐ)—, —N(R₃ₐ)—C(O)—, —N(R_{3b})—C(O)—O—, —N(R_{3b})—C(O)—NR₃ₐ—, —SO₂N(R₃ₐ)— or —N(R₃ₐ)SO₂—, where R₃ₐ is selected from the group consisting of hydrogen or (1-2C)alkyl;
Q₂ is selected from the group consisting of hydrogen, (1-4C)alkyl, (3-6C)cycloalkyl, (3-6C)cycloalkyl(1-2C)alkyl, phenyl, phenyl(1-4C)alkyl, 4- to 6-membered heterocyclyl, [4- to 6-membered heterocyclyl](1-2C)alkyl, 5- to 10-membered heteroaryl and [5- to 10-membered heteroaryl](1-2C)alkyl, wherein any alkyl, cycloalkyl, cycloalkyl-alkyl, phenyl, phenylalkyl, heterocyclyl, heterocyclyl-alkyl, heteroaryl or heteroaryl-alkyl in Q₂ is optionally substituted with one or more R₃ₑ groups; and
each R₃ₑ group present is independently selected from the group consisting of hydroxy, cyano, oxo, halogen, (1-4C)alkyl, (1-4C)alkoxy, —NR_{3d}R₃ₑ, —C(O)—R_{3d}, —C(O)—OR_{3d}, —O—C(O)—R_{3d}, —C(O)—NR_{3d}R₃ₑ, —N(R₃ₑ)C(O)—R_{3d}, —S(O)₀₋₂R_{3d}—, —S(O)₂NR_{3d}R₃ₑ, —N(R₃ₑ)—S(O)₂R_{3d}, phenyl, 5 or 6-membered heteroaryl, or 4 to 6-membered heterocyclyl,
wherein R_{3d} and R₃ₑ are each independently hydrogen or (1-2C)alkyl; and wherein any alkyl, alkoxy, cycloalkyl, cycloalkyl-alkyl, phenyl, heteroaryl or heterocyclyl group present in a R_{3c} group is optionally further substituted by one or more substituents independently selected from hydroxy, cyano, halogen, (1-2C)alkyl, or —OR_{3f}, wherein R₃ₐ is selected from hydrogen and (1-2C)alkyl;
(24) R₃ is cyano, or a group of the formula

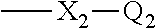

wherein:
X₂ is absent or is selected from the group consisting of —O—, —C(O)—, —NR₃ₐ—, —C(O)—O—, —O—C(O)—, —S(O)₀₋₂—, —C(O)—N(R₃ₐ)—, —N(R₃ₐ)—C(O)—, —N(R_{3b})—C(O)—O—, —N(R_{3b})—C(O)—NR₃ₐ—, —SO₂N(R₃ₐ)— or —N(R₃ₐ)SO₂—, where R₃ₐ is selected from the group consisting of hydrogen or (1-2C)alkyl;
Q₂ is selected from the group consisting of hydrogen, (1-4C)alkyl, (3-6C)cycloalkyl, (3-6C)cycloalkyl(1-2C)alkyl, phenyl, phenyl(1-4C)alkyl, 4- to 6-membered heterocyclyl, [4- to 6-membered heterocyclyl](1-2C)alkyl, 5- to 10-membered heteroaryl and [5- to 10-membered heteroaryl](1-2C)alkyl, wherein any alkyl, cycloalkyl, cycloalkyl-alkyl, phenyl, phenylalkyl, heterocyclyl, heterocyclyl-alkyl, heteroaryl or heteroaryl-alkyl in Q₂ is optionally substituted with one or more R₃, groups; and
each R_{3c} group present is independently selected from the group consisting of hydroxy, cyano, oxo, halogen, (1-4C)alkyl, (1-4C)alkoxy, —NR_{3d}R₃ₑ, —C(O)—R_{3d}, —S(O)₀₋₂R_{3d}—, —S(O)₀₋₂R_{3d}—, phenyl, 5 or 6-membered heteroaryl, or 4 to 6-membered heterocyclyl,
wherein R_{3d} and R₃ₑ are each independently hydrogen or (1-2C)alkyl; and wherein any alkyl, alkoxy, phenyl, heteroaryl or heterocyclyl group present in a R_{3c} group, is optionally further substituted by one or more substituents independently selected from hydroxy, cyano, halogen, (1-2C)alkyl, or —OR_{3f}, wherein R_{3f} is selected from hydrogen and (1-2C)alkyl;
(25) R₃ is cyano, or a group of the formula

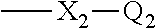

wherein:
X₂ is absent or is selected from the group consisting of —O—, —C(O)—, —NR₃ₐ—, —C(O)—O—, —S(O)₀₋₂—, —C(O)—N(R₃ₐ)—, —N(R₃ₐ)—C(O)—, —N(R_{3b})—C(O)—O—, —N(R_{3b})—C(O)—NR₃ₐ—, —SO₂N(R₃ₐ)— or —N(R₃,)SO₂—, where R₃ₐ is selected from the group consisting of hydrogen or methyl;
Q₂ is selected from the group consisting of hydrogen, (1-3C)alkyl, (3-6C)cycloalkyl, (3-6C)cycloalkyl(1-2C)alkyl, phenyl, phenyl(1-4C)alkyl, 4- to 6-membered heterocyclyl, [4- to 6-membered heterocyclyl](1-2C)alkyl, 5- to 10-membered heteroaryl and [5- to 10-membered heteroaryl](1-2C)alkyl, wherein any alkyl, cycloalkyl, cycloalkyl-alkyl, phenyl, phenylalkyl, heterocyclyl, heterocyclyl-alkyl, heteroaryl or heteroaryl-alkyl in Q₂ is optionally substituted with one or more R_{3c} groups; and each R₃, group present is independently selected from the group consisting of hydroxy, cyano, oxo, halogen, (1-4C)alkyl, (1-4C)alkoxy, —C(O)—R_{3d}, —C(O)—OR_{3d}, —O—C(O)—R_{3d}, —C(O)—NR_{3d}R₃ₑ, —N(R₃ₑ)C(O)—R_{3d}, —S(O)₀₋₂R_{3d}—, phenyl, 5 or 6-membered heteroaryl, or 4 to 6-membered heterocyclyl,
wherein R_{3d} and R₃ₑ are each independently hydrogen or (1-2C)alkyl; and wherein any alkyl, alkoxy, phenyl, heteroaryl or heterocyclyl group present in a R_{3c} group, is optionally further substituted by one or more substituents independently selected from hydroxy or halogen;
(26) R₃ is cyano, or a group of the formula

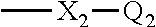

wherein:
X₂ is absent or is selected from the group consisting of —O—, —NR₃ₐ—, —C(O)—O—, —C(O)—N(R₃ₐ)—, —N(R₃ₐ)—C(O)—, —N(R_{3b})—C(O)—NR₃ₐ— or —N(R₃ₐ)SO₂—, where R₃ₐ is selected from the group consisting of hydrogen or methyl;
Q₂ is selected from the group consisting of hydrogen, (1-3C)alkyl, (3-6C)cycloalkyl, (3-6C)cycloalkyl(1-2C)alkyl, phenyl, phenyl(1-4C)alkyl, 4- to 6-membered heterocyclyl, [4- to 6-membered heterocyclyl](1-2C)alkyl, 5- to 10-membered heteroaryl and [5- to 10-membered heteroaryl](1-2C)alkyl, wherein any alkyl, cycloalkyl, cycloalkyl-alkyl, phenyl, phenylalkyl, heterocyclyl, heterocyclyl-alkyl, heteroaryl or heteroaryl-alkyl in Q₂ is optionally substituted with one or more R_{3c} groups; and
each R_{3c} group present is independently selected from the group consisting of hydroxy, cyano, oxo, halogen, (1-4C)alkyl, (1-4C)alkoxy, —C(O)—R_{3d}, —S(O)₀₋₂R_{3d}—, or 5 or 6-membered heteroaryl;
wherein R_{3d} and R₃ₑ are each independently hydrogen or (1-2C)alkyl; and wherein any alkyl, alkoxy, phenyl, heteroaryl or heterocyclyl group present in a R_{3c} group, is optionally further substituted by one or more substituents independently selected from hydroxy or halogen;
(27) R₃ is cyano, or a group of the formula

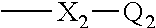

wherein:
X₂ is absent or is selected from the group consisting of —O—, —NRaa-, —C(O)—O—, —C(O)—N(R₃ₐ)—, —N(R₃ₐ)—C(O)—, —N(R_{3b})—C(O)—NR₃ₐ— or —N(R₃ₐ)SO₂—, where R₃ₐ is selected from the group consisting of hydrogen or methyl;
Q₂ is selected from the group consisting of hydrogen, (1-3C)alkyl, (3-6C)cycloalkyl, (3-6C)cycloalkyl(1-2C)alkyl, phenyl, phenyl(1-4C)alkyl, 4- to 6-membered heterocyclyl, [4- to 6-membered heterocyclyl](1-2C)alkyl, 5- to 10-membered heteroaryl and [5- to 10-membered heteroaryl](1-2C)alkyl, wherein any alkyl, cycloalkyl, cycloalkyl-alkyl, phenyl, phenylalkyl, heterocyclyl, heterocyclyl-alkyl, heteroaryl or heteroaryl-alkyl in Q₂ is optionally substituted with one or more R₃, groups; and
each R₃, group present is independently selected from the group consisting of hydroxy, cyano, oxo, halogen, (1-4C)alkyl, (1-4C)alkoxy, —C(O)—R_{3d}, —S(O)₀₋₂R_{3d}—, or 5 or 6-membered heteroaryl;
wherein R_{3d} and R₃ₑ are each independently hydrogen or methyl;
and wherein any alkyl, alkoxy, phenyl, heteroaryl or heterocyclyl group present in a R_{3c} group, is optionally further substituted by one or more substituents independently selected from hydroxy or halogen;
(28) R₃ is cyano, or a group of the formula

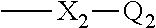

wherein:
X₂ is absent or is selected from the group consisting of —O—, —N(R₃ₐ)—C(O)—, —N(R_{3b})—C(O)—NR₃ₐ— or —N(R₃ₐ)SO₂—, where R₃ₐ is selected from the group consisting of hydrogen or methyl;
Q₂ is selected from the group consisting of hydrogen, (1-3C)alkyl, (3-6C)cycloalkyl, (3-6C)cycloalkyl(1-2C)alkyl, phenyl, phenyl(1-4C)alkyl, 4- to 6-membered heterocyclyl, [4- to 6-membered heterocyclyl](1-2C)alkyl, 5- to 10-membered heteroaryl and [5- to 10-membered heteroaryl](1-2C)alkyl, wherein any alkyl, cycloalkyl, cycloalkyl-alkyl, phenyl, phenylalkyl, heterocyclyl, heterocyclyl-alkyl, heteroaryl or heteroaryl-alkyl in Q₂ is optionally substituted with one or more R_{3c} groups; and
each R_{3c} group present is independently selected from the group consisting of hydroxy, cyano, oxo, halogen, (1-4C)alkyl, (1-4C)alkoxy, —C(O)—R_{3d} or —S(O)o. 2R_{3d}—;
wherein R_{3d} and R₃ₑ are each independently hydrogen or methyl;
and wherein any alkyl, alkoxy, phenyl, heteroaryl or heterocyclyl group present in a R₃ₑ group, is optionally further substituted by one or more substituents independently selected from hydroxy or halogen;
(29) R₃ is a group of the formula:

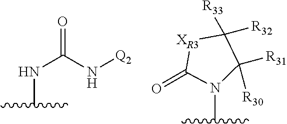

wherein:
Q₂ is as defined in any one of paragraphs (7) to (14) above;
X_{R3} is O, NH or N(1-2C)alkyl;
R₃₀, R₃₁, R₃₂ and R₃₃ are each independently selected from hydrogen, (1-6C)alkyl, (3-6C)cycloalkyl, (3-6C)cycloalkyl(1-2C)alkyl, 4- to 6-membered heterocyclyl or [4- to 6-membered heterocyclyl](1-2C)alkyl, and wherein any alkyl, cycloalkyl, cycloalkyl-alkyl, heterocyclyl and heterocyclyl-alkyl is optionally substituted with one or more R₃ₑ substituents;
or either R₃₀ and R₃₁ or R₃₂ and R₃₃ are linked such that, together with the carbon atom to which they are attached, they form a spiro fused 3 to 6 membered cycloalkyl or heterocyclyl ring, which is optionally substituted with one or more R_{3c} substituents;
wherein each R₃, present is independently selected from hydroxy, cyano, oxo, halogen, (1-4C)alkyl, (1-4C)alkoxy, (3-6C)cycloalkyl, (3-6C)cycloalkyl(1-2C)alkyl, —NR_{3d}R₃ₑ, —C(O)—R_{3d}, —C(O)—OR_{3d}, —O—C(O)—R_{3d}, —C(O)—NR_{3d}R₃ₑ, —N(R₃ₑ)C(O)—R_{3d}, —S(O)₀₋₂R_{3d}—, —S(O)₂NR_{3d}R₃ₑ, —N(R₃ₑ)—S(O)₂R_{3d} wherein R_{3d} and R₃ₑ are each independently hydrogen, (1-4C)alkyl, (3-6C)cycloalkyl, or (3-6C)cycloalkyl(1-2C)alkyl;
(30) R₃ is a group of the formula:

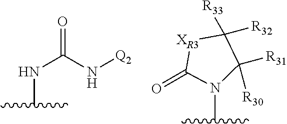

wherein:
Q₂ is selected from hydrogen, (1-6C)alkyl, (3-6C)cycloalkyl, (3-6C)cycloalkyl(1-2C)alkyl, 4- to 6-membered heterocyclyl or [4- to 6-membered heterocyclyl](1-2C)alkyl, each of which is optionally substituted by one or more R_{K}o substituents;
X_{R3} is O, NH or N(1-2C)alkyl;
R₃₀, R₃₁, R₃₂ and R₃₃ are each independently selected from hydrogen, (1-6C)alkyl, (3-6C)cycloalkyl, (3-6C)cycloalkyl(1-2C)alkyl, 4- to 6-membered heterocyclyl or [4- to 6-membered heterocyclyl](1-2C)alkyl, and wherein any alkyl, cycloalkyl, cycloalkyl-alkyl, heterocyclyl and heterocyclyl-alkyl is optionally substituted with one or more R_{3c} substituents;
or either R₃₀ and R₃₁ or R₃₂ and R₃₃ are linked such that, together with the carbon atom to which they are attached, they form a spiro fused 3 to 6 membered cycloalkyl or heterocyclyl ring, which is optionally substituted with one or more R_{3c} substituents;
wherein each R_{3c} present is independently selected from hydroxy, cyano, oxo, halogen, (1-4C)alkyl, (1-4C)alkoxy, (3-6C)cycloalkyl, (3-6C)cycloalkyl(1-2C)alkyl, —NR_{3d}R₃ₑ, —C(O)—Rad, —C(O)—OR_{3d}, —O—C(O)—R_{3d}, —C(O)—NR_{3d}R₃ₑ, —N(R₃ₑ)C(O)—R_{3d}, —S(O)₀₋₂R_{3d}—, —S(O)₂NR_{3d}R₃ₑ, —N(R₃ₑ)—S(O)₂R_{3d}:
wherein R_{3d} and R₃ₑ are each independently hydrogen, (1-4C)alkyl, (3-6C)cycloalkyl, or (3-6C)cycloalkyl(1-2C)alkyl;
(31) R₃ is a group of the formula:

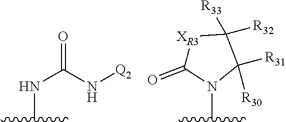

wherein:
Q₂ is selected from (1-6C)alkyl, (3-6C)cycloalkyl or 4- to 6-membered heterocyclyl each of which is optionally substituted by one or more R_{3K} substituents;
X_{R3} is O, NH or N(CH₃);
R₃₀, R₃₁, R₃₂ and R₃₃ are each independently selected from hydrogen, (1-6C)alkyl, (3-6C)cycloalkyl, (3-6C)cycloalkyl(1-2C)alkyl, 4- to 6-membered heterocyclyl or [4- to 6-membered heterocyclyl](1-2C)alkyl, and wherein any alkyl, cycloalkyl, cycloalkyl-alkyl, heterocyclyl and heterocyclyl-alkyl is optionally substituted with one or more Ra, substituents;
or either R₃₀ and R₃₁ or R₃₂ and R₃₃ are linked such that, together with the carbon atom to which they are attached, they form a spiro fused 3 to 6 membered cycloalkyl or heterocyclyl ring, which is optionally substituted with one or more R₃ₑ substituents;
wherein each R₃ₑ present is independently selected from hydroxy, cyano, oxo, halogen, (1-4C)alkyl, (1-4C)alkoxy, —NR_{3d}R₃ₑ, —C(O)—R_{3d}, —C(O)—OR_{3d}, —O—C(O)—R_{3d}, —C(O)—NR_{3d}R₃ₑ, or —N(R₃ₑ)C(O)—R_{3d};
wherein R_{3d} and R₃ₑ are each independently hydrogen or (1-4C)alkyl;
(32) R₃ is a group of the formula:

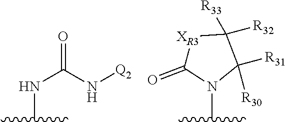

wherein:
Q₂ is selected from (1-6C)alkyl, (3-6C)cycloalkyl or 4- to 6-membered heterocyclyl each of which is optionally substituted by one or more R_{3K} substituents;
X_{R3} is O, NH or N(CH₃);
R₃₀, R₃₁, R₃₂ and R₃₃ are each independently selected from hydrogen or (1-6C)alkyl optionally substituted with one or more R₃ₑ substituents;
or either R₃₀ and R₃₁ or R₃₂ and R₃₃ are linked such that, together with the carbon atom to which they are attached, they form a spiro fused 3 to 6 membered cycloalkyl or heterocyclyl ring, which is optionally substituted with one or more R₃ₑ substituents;
wherein each R₃ₑ present is independently selected from hydroxy, cyano, halogen, (1-4C)alkyl, or (1-4C)alkoxy;
(33) R₃ is a group of the formula:

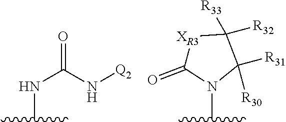

wherein:
Q₂ is selected from (1-6C)alkyl, (3-6C)cycloalkyl or 4- to 6-membered heterocyclyl each of which is optionally substituted by one or more R_{3c} substituents;
X_{R3} is O, NH or N(CH₃);
R₃₀, R₃₁, R₃₂ and R₃₃ are each independently selected from hydrogen or (1-6C)alkyl optionally substituted with one or more R₃, substituents;
or either R₃₀ and R₃₁ or R₃₂ and R₃₃ are linked such that, together with the carbon atom to which they are attached, they form a spiro fused 3 to 6 membered cycloalkyl ring, which is optionally substituted with one or more R_{3c} substituents; wherein each R_{3c} present is independently selected from hydroxy, cyano, halogen, (1-4C)alkyl, or (1-4C)alkoxy;
(34) R₃ is a group of the formula:

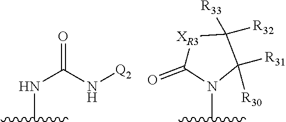

wherein:
Q₂ is selected from (1-6C)alkyl, (3-6C)cycloalkyl or 4- to 6-membered heterocyclyl each of which is optionally substituted by one or more R_{3c} substituents;
X_{R3} is O, NH or N(CH₃);
R₃₂ and R₃₃ are hydrogen;
R₃₀ and R₃₁ are each independently selected from hydrogen or (1-6C)alkyl optionally substituted with one or more R_{3c} substituents;
or R₃₀ and R₃₁ are linked such that, together with the carbon atom to which they are attached, they form a spiro fused 3 to 6 membered cycloalkyl ring, which is optionally substituted with one or more R_{3c} substituents;
wherein each R₃ₑ present is independently selected from hydroxy, cyano, halogen, (1-4C)alkyl, or (1-4C)alkoxy; or
(35) R₃ is a group of the formula:

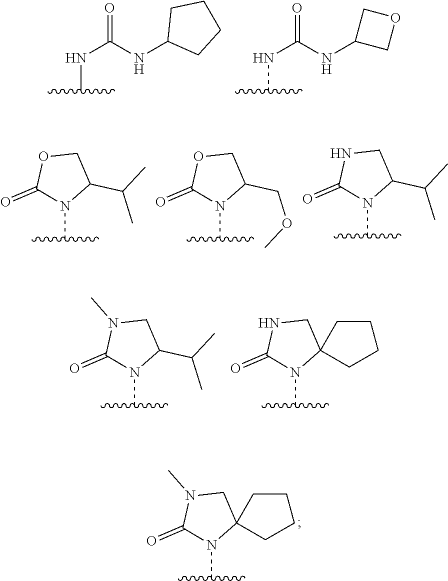

(36) Q, is selected from hydrogen or:
   (i) a group of the formula:

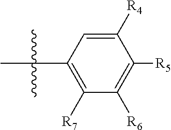

wherein:
R₄, R₅ and R₇ are each independently selected from hydrogen, halo, cyano, or a group of the formula:

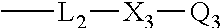

wherein:
L₂ is absent or (1-3C)alkylene;
X₃ is absent or is selected from the group consisting of —O—, —NR₄ₐ—, —C(O)—O—, —O—C(O)—, —S(O)₀₋₂—, —C(O)—N(R₄ₐ)—, —N(R₄ₐ)—C(O)—, —SO₂N(R₄ₐ)— or —N(R₄ₐ)SO₂—, where R₄ₐ and R_{4b} are independently selected from the group consisting of hydrogen or (1-2C)alkyl;
Q₃ is selected from the group consisting of hydrogen, (1-4C)alkyl, (3-6C)cycloalkyl, (3-6C)cycloalkyl(1-2C)alkyl, phenyl, phenyl(1-2C)alkyl, 3 to 8-membered heterocyclyl, [3 to 8-membered heterocyclyl](1-2C)alkyl, 5 or 6-membered heteroaryl and 5 or 6-membered heteroaryl(1-2C)alkyl, wherein any alkyl, cycloalkyl, cycloalkylalkyl, aryl, arylalkyl, heterocyclyl, heterocyclyl-alkyl, heteroaryl or heteroaryl-alkyl in Q₃ is optionally substituted with one or more R₄ₑ groups; and each R_{4c} group present is independently selected from the group consisting of hydroxy, cyano, oxo, halogen, (1-4C)alkyl, (1-4C)alkoxy, (3-6C)cycloalkyl, (3-6C)cycloalkyl(1-2C)alkyl, —NR_{4d}R₄ₑ, —C(O)—R_{4d}, —C(O)—OR_{4d}, —O—C(O)—R_{4d}, —C(O)—NR_{4d}R₄ₑ, —N(R₄ₑ)C(O)—R_{4d}, —S(O)₀₋₂R_{4d}—, —S(O)₂NR_{4d}R₄ₑ, or —N(R₄ₑ)—S(O)₂R_{4d},
wherein R_{4d} and R₄ₑ are each independently hydrogen or (1-4C)alkyl; and wherein any alkyl, alkoxy, cycloalkyl, cycloalkyl-alkyl group present in a R₄ₑ group, or any alkyl group present in a R_{4d} or R₄ₑ group, is optionally further substituted by one or more substituents independently selected from hydroxy, cyano, halogen, R_{4f}, —OR_{4f}, —NR_{4f}R_{4g}—C(O)OR_{4f}, —OC(O)R_{4f}, —C(O)NR_{4f}R_{4g}, —NR_{4g}C(O)R_{4f}, —S(O)O-2—R_{4f} and —C(O)—R_{4f}, wherein R_{4f} and R_{4g} are both independently selected from hydrogen or (1-2C)alkyl;
R₅ is:
   a) hydrogen, halo, cyano; or
   b) a group of the formula:

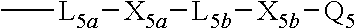

wherein:
L₅ₐ is absent or (1-3C)alkylene;
X₅ₐ is absent or is selected from the group consisting of —O—, —C(O)—, —NR₅ₐ₁—, —C(O)—O—, —O—C(O)—, —S(O)₀₋₂—, —C(O)—N(R₅ₐ₁)—, —N(R₅ₐ₁)—C(O)—, —SO₂N(R₅ₐ₁)— or —N(R₅ₐ₁)SO₂—, where R₅ₐ₁ and R₅ₐ₂ are independently selected from the group consisting of hydrogen or (1-4C)alkyl;
L_{5b} is absent or (1-4C)alkylene;
X_{5b} is absent or is selected from the group consisting of —O—, —C(O)—, —NR_{5b1}—, —C(O)—O—, —O—C(O)—, —S(O)₀₋₂—, —C(O)—N(R_{5b1})—, —N(R_{5b1})—C(O)—, —O—C(O)—N(R_{5b1})—, —N(R_{5b1})—C(O)—O—, —N(R_{5b2})—C(O)—NR_{5b1}—, —SO₂N(R_{5b1})— or —N(R_{5b1})SO₂—, where R_{5b1} and R_{5b2} are independently selected from the group consisting of hydrogen or (1-4C)alkyl;
Q₅ is selected from the group consisting of hydrogen, (1-6C)alkyl, (3-8C)cycloalkyl, (3-8C)cycloalkyl(1-4C)alkyl, phenyl, phenyl(1-2C)alkyl, 3 to 8-membered heterocyclyl, [3 to 8-membered heterocyclyl](1-2C)alkyl, 5 or 6-membered heteroaryl and 5 or 6-membered heteroaryl(1-2C)alkyl and wherein Q₅ is optionally substituted with one or more R_{5c} groups;
and each R₅. group present is independently selected from the group consisting of hydroxy, cyano, oxo, halogen, (1-6C)alkyl, (1-6C)alkoxy, (3-8C)cycloalkyl (including spiro-fused (3-8C)cycloalkyl), or (3-8C)cycloalkyl(1-2C)alkyl, —NR_{5d}R₅ₑ, —C(O)—R_{5d}, —C(O)—OR_{5d}, —O—C(O)—R_{5d}, —C(O)—NR_{5d}R₅ₑ, —N(R₅ₑ)C(O)—R_{5d}, —S(O)₀₋₂R_{5d}—, —S(O)₂NR_{5d}R₅ₑ, —N(R₅ₑ)—S(O)₂R_{5d}, phenyl, 5 or 6-membered heteroaryl, or 4 to 8-membered heterocyclyl,
wherein R_{5d} and R₅ₑ are each independently hydrogen, (1-6C)alkyl, (3-6C)cycloalkyl, or (3-6C)cycloalkyl(1-2C)alkyl;
and wherein any alkyl, alkoxy, cycloalkyl, cycloalkyl-alkyl, phenyl, heteroaryl or heterocyclyl group present in a R_{5c} group, or any alkyl, cycloalkyl or cycloalkyl-alkyl group present in a R_{5d} or R₅ₑ group, is optionally further substituted by one or more substituents independently selected from hydroxy, cyano, halo, R_{5f}, —OR_{5f}, —NR_{5f}R_{5g} and —C(O)—R_{5f}, wherein R₅ᵣ and R₅ₑ are both independently selected from hydrogen, (1-4C)alkyl, hydroxy(1-4C)alkyl, halo(1-4C)alkyl, or (1-2C)alkoxy(1-4C)alkyl;
or R₄ and R₅, R₅ and R₆, or R₆ and R₇ are linked to form a fused phenyl, 5- or 6-membered heteroaryl or 5 to 7-membered heterocyclic ring, which is optionally substituted by one or more substituent groups selected from hydroxy, cyano, halo, (1-2C)alkyl, (1-2C)haloalkyl, (1-2C)alkoxy or (1-2C)haloalkoxy;
(ii) a group of the formula:

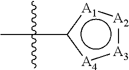

wherein:
A₁ is selected from N, NR_{1N}, O, S or CR₇;
A₂ is selected from N, NR_{1N}, O, S or CR₄;
A₃ is selected from N, NR_{1N}, O, S or CR₅;
A₄ is selected from N, NR_{1N}, O, S or CR₇;
R_{1N} is selected from hydrogen, (1-4C)alkyl, (2-4C)alkanoyl, phenyl, benzyl, (3-8C)cycloalkyl, (3-8C)cycloalkyl-(1-2C)alkyl, a carbon-linked 4 to 7 membered heterocyclyl, or 4 to 7 membered heterocyclyl(1-2C)alkyl, and wherein any alkyl, alkanoyl, cycloalkyl, cycloalkyl-alkyl, phenyl, benzyl, heterocyclyl, heterocyclyl-alkyl group is optionally substituted with one or more R_{5c} groups defined above;
R₄, R₅ and R₇ are as defined above;
and with the proviso that:
   (i) only one of A₁, A₂, A₃ and A₄ can be O or S;
   (ii) only one of A₁, A₂, A₃ and A₄ can be NR_{1N};
   (iii) 1 to 3 of A₁, A₂, A₃ and A₄ can be N;
(iii) a group of the formula:

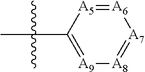

wherein:
A₉ is selected from N or CH;
A₆ is selected from N or CR₄;
A₇ is selected from N or CR₅;
A₉ is selected from N or CR₆;
A₉ is selected from N or CR₇;
R₄, R₅, R₆ and R₇ are as defined above;
and with the proviso that only one of A₅, A₆, A₇, A₈ and A₉ can be N;
(iv) a group of the formula:

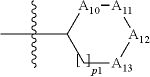

wherein:
p1 is 0 or 1;
A₁₀ is selected from NH, CH₂ or CHR₇;
A₁₁ is selected from NR_{4N}, CH₂ or CHR₄;
A₁₂ is selected from NR_{5N}, CH₂ or CHR₅;
A₁₃ is selected from NR_{6N}, CH₂ or CHR₆;
R₄, R₅, R₆ and R₇ are as defined above;
R_{4N} and R_{6N} are each independently selected from hydrogen or a group of the formula:

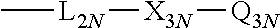

wherein:
L_{2N} is absent or (1-3C)alkylene;
X_{3N} is absent or is selected from the group consisting of:
   (i) —C(O)—, —S(O)₀₋₂— or —C(O)—N(R₄ₐ)— when L₂ is absent; or
   (ii) —O—, —C(O)—, —NR₄ₐ—, —C(O)—O—, —O—C(O)—, —S(O)₀₋₂—, —C(O)—N(R₄ₐ)—, —N(R₄ₐ)—C(O)—, —O—C(O)—N(R₄ₐ)—, —N(R₄)—C(O)—O—, —N(R_{4b})—C(O)—NR₄ₐ—, —SO₂N(R₄ₐ)— or —N(R₄ₐ)SO₂— when L₂ is (1-3C)alkylene;
   wherein R₄ₐ and R_{4b} are as defined above;
Q_{3N} is selected from the group consisting of hydrogen, (1-6C)alkyl, (3-6C)cycloalkyl, (3-6C)cycloalkyl(1-2C)alkyl, heterocyclyl, heterocyclyl(1-2C)alkyl, heteroaryl and heteroaryl(1-2C)alkyl, wherein any alkyl, cycloalkyl, cycloalkylalkyl, heterocyclyl, heterocyclylalkyl, heteroaryl or heteroarylalkyl in Q₃ is optionally substituted with one or more R₄ₑ groups; and each R₄ₑ group present is as defined above; R_{5N} is:
   a) hydrogen;
   b) a group of the formula:

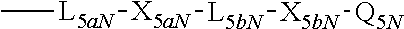

wherein:
L_{5aN} is absent or (1-3C)alkylene;
X_{5aN} is absent or is selected from the group consisting of:
(i) —C(O)—, —S(O)₀₋₂—, —C(O)—N(R₅ₐ₁)— when L_{5aN} is absent; or
(ii) —O—, —C(O)—, —NR₅ₐ₁—, —C(O)—O—, —O—C(O)—, —S(O)₀₋₂—, —C(O)—N(R₅ₐ₁)—, —N(R₅ₐ₁)—C(O)—, —O—C(O)—N(R₅ₐ₁)—, —N(R₅ₐ₁)—C(O)—O—, —N(R₅ₐ₂)—C(O)—NR₅ₐ₁—, —SO₂N(R₅ₐ) or —N(R₅ₐ₁)SO₂— when L_{5aN} is present;
where R₅,1 and R₅ₐ₂ are as defined above;
L_{5bN} is absent or (1-2C)alkylene;
X_{5bN} is absent or is selected from the group consisting of —O—, —C(O)—, —NR_{5b1}—, —C(O)—O—, —O—C(O)—, —S(O)₀₋₂—, —C(O)—N(R_{5b1})—, —N(R_{5b1})—C(O)—, —SO₂N(R_{5b1})— or —N(R_{5b1})SO₂—, where R_{5b1} and R_{5b2} are independently selected from the group consisting of hydrogen or (1-4C)alkyl;
Q_{5N} is selected from the group consisting of:
(i) hydrogen;
(ii) (1-4C)alkyl, (3-6C)cycloalkyl, (3-6C)cycloalkyl(1-1C)alkyl, phenyl, phenyl(1-2C)alkyl, 5- or 6-membered heteroaryl, 5- or 6-membered heteroaryl(1-2C)alkyl;
(iii) a carbon-linked 4 to 8 membered heterocyclyl (when -L_{5aN}, X_{5aN}, L_{5bN}, and X_{5bN} are absent) or a 4 to 8 membered heterocyclyl (when one or more of -L_{5aN}, X_{5aN}, L_{5bN}, and X_{5bN} are present), or [4 to 8 membered heterocyclyl](1-2C)alkyl;
and wherein Q_{5N} is optionally substituted with one or more R_{5c} groups as defined above;
and with the proviso that one or two of A₁₀, A₁₁, A₁₂ and A₁₃ can be N;
(v) a group of the formula:

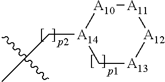

wherein:
p1, A₁₀, A₁₁, A₁₂, and A₁₃ are as defined above;
p2 is 1 to 5;
A₁₄ is C or N;
and with the proviso that one of A₁₀, A₁₁, A₁₂ and A₁₃ can be N.
(37) Q₁ is selected from hydrogen or:
   (i) a group of the formula:

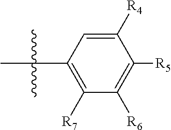

wherein:
R₄, R₆ and R₇ are each independently selected from hydrogen, or a group of the formula:

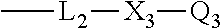

wherein:
L₂ is absent or (1-3C)alkylene;
X₃ is absent or is selected from the group consisting of —O—, —NR₄ₐ—, —S(O)₀₋₂—, —C(O)—N(R₄ₐ)—, —N(R₄ₐ)—C(O)—, —SO₂N(R₄ₐ)— or —N(R₄ₐ)SO₂—, where R₄ₐ and R_{4b} are independently selected from the group consisting of hydrogen or (1-2C)alkyl;
Q₃ is selected from the group consisting of hydrogen, (1-4C)alkyl, (3-6C)cycloalkyl, (3-8C)cycloalkyl(1-4C)alkyl, phenyl, 3 to 8-membered heterocyclyl, 5 or 6-membered heteroaryl or 5 or 6-membered heteroaryl(1-4C)alkyl wherein any alkyl, cycloalkyl, cycloalkylalkyl, phenyl, heterocyclyl, or heteroaryl in Q₃ is optionally substituted with one or more R_{4c} groups; and
each R_{4c} group present is independently selected from the group consisting of hydroxy, cyano, oxo, halogen, (1-4C)alkyl, (1-4C)alkoxy, (3-6C)cycloalkyl, (3-6C)cycloalkyl(1-2C)alkyl, —NR_{4d}R₄ₑ, —C(O)—R_{4d}, —C(O)—OR_{4d}, —O—C(O)—R_{4d}, —C(O)—NR_{4d}R₄ₑ, —N(R₄ₑ)C(O)—R_{4d}, —S(O)₀₋₂R_{4d}—, —S(O)₂NR_{4d}R₄ₑ, or —N(R₄ₑ)—S(O)₂R_{4d},
wherein R_{4d} and R₄ₑ are each independently hydrogen or (1-4C)alkyl; and wherein any alkyl, alkoxy, cycloalkyl, cycloalkyl-alkyl, phenyl, heteroaryl or heterocyclyl group present in a R_{4c} group, or any alkyl group present in a R_{4d} or R₄ₑ group, is optionally further substituted by one or more substituents independently selected from hydroxy, cyano, halogen, (1-2C)alkyl, —OR_{4f}, —NR_{4f}R_{4g}, —S(O)₀₋₂—R_{4f} and —C(O)—R_{4f}, wherein R_{4f} and R_{4g} are both independently selected from hydrogen and (1-2C)alkyl;
R₅ is:
   a) hydrogen, halo, cyano; or
   b) a group of the formula:

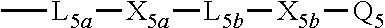

wherein:
La is absent or (1-3C)alkylene;
X₅ₐ is absent or is selected from the group consisting of —O—, —C(O)—, —NR₅ₐ₁—, —S(O)₀₋₂—, —C(O)—N(R₅ₐ₁)—, —N(R₅ₐ₁)—C(O)—, —SO₂N(R₅ₐ₁)— or —N(R₅ₐ₁)SO₂—, where R₅ₐ₁ and R₅ₐ₂ are independently selected from the group consisting of hydrogen or (1-2C)alkyl;
L_{5b} is absent or (1-4C)alkylene;
X_{5b} is absent or is selected from the group consisting of —O—, —C(O)—, —NR_{5b}—, —S(O)₀₋₂—, —C(O)—N(R_{5b1})—, —N(R_{5b1})—C(O)—, —SO₂N(R_{5b1})— or —N(Rbl)SO₂—, where R_{5b1} and R_{5b2} are independently selected from the group consisting of hydrogen or (1-2C)alkyl;
Q₅ is selected from the group consisting of hydrogen, (1-6C)alkyl, (3-8C)cycloalkyl, phenyl, 3 to 8-membered heterocyclyl, or 5 or 6-membered heteroaryl and wherein Q₅ is optionally substituted with one or more R_{5c} groups;
and each R_{5c} group present is independently selected from the group consisting of hydroxy, cyano, halogen, (1-6C)alkyl, (1-6C)alkoxy, (3-8C)cycloalkyl (including spiro-fused (3-8C)cycloalkyl), (3-8C)cycloalkyl(1-2C)alkyl, —NR_{5d}R₅ₑ, —C(O)—R_{5d}, —C(O)—NR_{5d}R₅ₑ, —N(R₅ₑ)C(O)—R_{5d}, —S(O)₀₋₂R_{5d}—, —S(O)₂NR_{5d}R₅ₑ, —N(R₅ₑ)—S(O)₂R_{5d}, phenyl, 5 or 6-membered heteroaryl, or 4 to 8-membered heterocyclyl,
wherein R_{5d} and R₅ₑ are each independently hydrogen, (1-6C)alkyl, (3-6C)cycloalkyl, or (3-6C)cycloalkyl(1-2C)alkyl;
and wherein any alkyl, alkoxy, cycloalkyl, cycloalkyl-alkyl, phenyl, heteroaryl or heterocyclyl group present in a R_{5c} group, or any alkyl, cycloalkyl or cycloalkyl-alkyl group present in a R_{5d} or R₅ₑ group, is optionally further substituted by one or more substituents independently selected from hydroxy, cyano, halo, R_{5f}, —OR_{5f}, —NR_{5f}R_{5g} and —C(O)—R_{5f}, wherein R_{5f} and R_{5g} are both independently selected from hydrogen, (1-4C)alkyl, hydroxy(1-4C)alkyl, halo(1-4C)alkyl, or (1-2C)alkoxy(1-4C)alkyl;
or R₄ and R₅, R₅ and R₆, or R₆ and R₇ are linked to form a fused phenyl, 5- or 6-membered heteroaryl or 5 to 7-membered heterocyclic ring, which is optionally substituted by one or more substituent groups selected from hydroxy, cyano, halo, (1-2C)alkyl, (1-2C)haloalkyl, (1-2C)alkoxy or (1-2C)haloalkoxy;
(ii) a group of the formula:

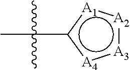

wherein:
A₁ is selected from N, NR_{1N}, O, S or CR₇;
A₂ is selected from N, NR_{1N}, O, S or CR₄;
A₃ is selected from N, NR_{1N}, O, S or CR₅;
A₄ is selected from N, NR_{1N}, O, S or CR₇;
R_{1N} is selected from hydrogen, (1-4C)alkyl, (2-4C)alkanoyl, phenyl, benzyl, (3-8C)cycloalkyl, (3-8C)cycloalkyl-(1-2C)alkyl, a carbon-linked 4 to 7 membered heterocyclyl, or 4 to 7 membered heterocyclyl(1-2C)alkyl, and wherein any alkyl, alkanoyl, cycloalkyl, cycloalkyl-alkyl, phenyl, benzyl, heterocyclyl, heterocyclyl-alkyl group is optionally substituted with one or more R_{5C} groups defined above;
R₄, R₅ and R₇ are as defined above;
and with the proviso that:
   (i) only one of A₁, A₂, A₃ and A₄ can be O or S;
   (ii) only one of A₁, A₂, A₃ and A₄ can be NR_{1N};
   (iii) 1 to 3 of A₁, A₂, A₃ and A₄ can be N;
(iii) a group of the formula:

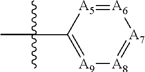

wherein:
A₅ is selected from N or CH;
A₉ is selected from N or CR₄;
A₇ is selected from N or CR₅;
A₈ is selected from N or CR₅;
A₉ is selected from N or CR₇;
R₄, R₅, R₆ and R₇ are as defined above;
and with the proviso that only one of A₅, A₆, A₇, A₈ and A₉ can be N;
(iv) a group of the formula:

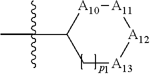

wherein:
p1 is 0 or 1;
A₁₀ is selected from NH, CH₂ or CHR₇;
A₁₁ is selected from NR_{4N}, CH₂ or CHR₄;
A₁₂ is selected from NR_{5N}, CH₂ or CHR₅;
A₁₃ is selected from NR_{6N}, CH₂ or CHRo;
R₄, R₅, R₆ and R₇ are as defined above;
R_{4N} and R_{6N} are each independently selected from hydrogen or a group of the formula:

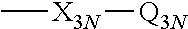

wherein:
X_{3N} is absent or is selected from the group consisting of: —C(O)—, —S(O)₀₋₂— or —C(O)—N(R₄ₐ)—;
   wherein R₄ₐ is as defined above;
Q_{3N} is selected from the group consisting of hydrogen, (1-4C)alkyl, (3-6C)cycloalkyl, (3-6C)cycloalkyl(1-2C)alkyl, heterocyclyl, heteroaryl and, wherein any alkyl, cycloalkyl, cycloalkylalkyl, heterocyclyl or heteroaryl in Q₃ is optionally substituted with one or more R₄ₑ groups; and each R₄, group present is as defined above;
R_{5N} is:
   a) hydrogen;
   b) a group of the formula:

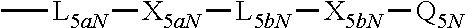

wherein:
L_{5aN} is absent or (1-3C)alkylene;
X_{5aN} is absent or is selected from the group consisting of:
(iii) —C(O)—, —S(O)₀₋₂—, —C(O)—N(R₅ₐ₁)— when L_{5aN} is absent; or
(iv) —O—, —C(O)—, —NR₅ₐ₁—, —C(O)—O—, —O—C(O)—, —S(O)₀₋₂—, —C(O)—N(R₅ₐ₁)—, —N(R₅ₐ₁)—C(O)—, —O—C(O)—N(R₅ₐ₁)—, —N(R₅ₐ₁)—C(O)—O—, —N(R₅ₐ₂)—C(O)—NR₅ₐ₁—, —SO₂N(R₅ₐ₁)— or —N(Rsa1)SO₂— when L_{5aN} is present;
where R₅ₐ₁ and R₅ₐ₂ are as defined above;
L_{5bN} is absent or (1-20)alkylene;
X_{5bN} is absent or is selected from the group consisting of —O—, —C(O)—, —NR_{5b1}—, —S(O)₀₋₂—, —C(O)—N(R_{5b1})—, —N(R_{5b1})—C(O)—, —SO₂N(R_{5b1})— or —N(R_{5b1})SO₂—, where R_{5b1} and R_{5b2} are independently selected from the group consisting of hydrogen or (1-2C)alkyl;
Q_{5N} is selected from the group consisting of:
(i) hydrogen;
(ii) (1-4C)alkyl, (3-6C)cycloalkyl, (3-6C)cycloalkyl(1-1C)alkyl, phenyl, phenyl(1-2C)alkyl, 5- or 6-membered heteroaryl, 5- or 6-membered heteroaryl(1-2C)alkyl;
and wherein Q_{5N} is optionally substituted with one or more R_{5c} groups as defined above;
and with the proviso that one or two of A₁₀, A₁₁, A₁₂ and A₁₃ can be N;
   (v) a group of the formula:

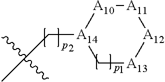

wherein:
p1, A₁₀, A₁₁, A₁₂, and A₁₃ are as defined above;
p2 is 1 to 4;
A₁₄ is C or N;
and with the proviso that one of A₁₀, A₁₁, A₁₂ and A₁₃ can be N.
(38) Q₁ is selected from hydrogen or:
   (i) a group of the formula:

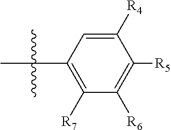

wherein:
R₄, R₅ and R₇ are each independently selected from hydrogen, or a group of the formula:

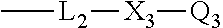

wherein:
L₂ is absent or (1-2C)alkylene;
X₃ is absent or is selected from the group consisting of —O—, —NR₄ₐ—, —C(O)—N(R₄ₐ)—, —N(R₄ₐ)—C(O)—, —SO₂N(R₄ₐ)— or —N(R₄ₐ)SO₂—, where R₄ₐ and R_{4b} are independently selected from the group consisting of hydrogen or (1-2C)alkyl;
Q₃ is selected from the group consisting of hydrogen, (1-4C)alkyl, (3-6C)cycloalkyl, (3-6C)cycloalkyl(1-4C)alkyl, phenyl, 3 to 8-membered heterocyclyl, 5 or 6-membered heteroaryl or 5 or 6-membered heteroaryl(1-4C)alkyl wherein any alkyl, cycloalkyl, cycloalkylalkyl, phenyl, heterocyclyl or heteroaryl in Q₃ is optionally substituted with one or more R₄ₑ groups; and
each R_{4c} group present is independently selected from the group consisting of hydroxy, cyano, oxo, halogen, (1-4C)alkyl, (1-4C)alkoxy, (3-6C)cycloalkyl, (3-6C)cycloalkyl(1-2C)alkyl, —NR_{4d}R₄ₑ, —C(O)—R_{4d}, —C(O)—OR_{4d}, —O—C(O)—R_{4d}, —C(O)—NR_{4d}R₄ₑ, —N(R₄ₑ)C(O)—R_{4d}, —S(O)₀₋₂R_{4d}—, —S(O)₂NR_{4d}R₄ₑ, or —N(R₄ₑ)—S(O)₂R_{4d},
wherein R_{4d} and R₄ₑ are each independently hydrogen or (1-2C)alkyl; and wherein any alkyl, alkoxy, cycloalkyl, cycloalkyl-alkyl, phenyl, heteroaryl or heterocyclyl group present in a R_{4c} group, or any alkyl group present in a R_{4d} or R₄ₑ group, is optionally further substituted by one or more substituents independently selected from hydroxy, cyano, halogen, (1-2C)alkyl, —OR_{4f}, —NR_{4f}R_{4g}, —S(O)₀₋₂—R_{4f} and —C(O)—R_{4f}, wherein R_{4f} and R_{4g} are both independently selected from hydrogen and (1-2C)alkyl;
R₅ is:
   a) hydrogen, halo, cyano; or
   b) a group of the formula:

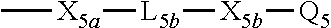

wherein:
X₅ₐ is absent or is selected from the group consisting of —O—, —C(O)—, —NR₅ₐ₁—, —S(O)₀₋₂—, —C(O)—N(R₅ₐ₁)—, —N(R₅ₐ)—C(O)—, —SO₂N(R₅ₐ₁)— or —N(R₅ₐ₁)SO₂—, where R₅ₐ₁ and R₅ₐ₂ are independently selected from the group consisting of hydrogen or (1-2C)alkyl;
L_{5b} is absent or (1-4C)alkylene;
X_{5b} is absent or is selected from the group consisting of —O—, —C(O)—, —NR_{5b}—, —S(O)₀₋₂—, —C(O)—N(R_{5b1})—, —N(R_{5b1})—C(O)—, —SO₂N(R_{5b1})— or —N(R_{5b1})SO₂—, where R_{5b1} and R_{5b2} are independently selected from the group consisting of hydrogen or (1-2C)alkyl;
Q₅ is selected from the group consisting of hydrogen, (1-6C)alkyl, (3-8C)cycloalkyl, phenyl, 3 to 8-membered heterocyclyl, or 5 or 6-membered heteroaryl and wherein Q₅ is optionally substituted with one or more R_{5c} groups;
and each R_{5c} group present is independently selected from the group consisting of hydroxy, cyano, halogen, (1-6C)alkyl, (1-6C)alkoxy, (3-8C)cycloalkyl (including spiro-fused (3-8C)cycloalkyl), (3-8C)cycloalkyl(1-2C)alkyl, —NR_{5d}R₅ₑ, —C(O)—R_{5d}, —C(O)—NR_{5d}R₅ₑ, —N(R₅ₑ)C(O)—R_{5d}, —S(O)₀₋₂R_{5d}—, —S(O)₂NR_{5d}R₅ₑ, —N(R₅ₑ)—S(O)₂R_{5d}, phenyl, 5 or 6-membered heteroaryl, or 4 to 8-membered heterocyclyl, wherein R_{5d} and R₅ₑ are each independently hydrogen, (1-6C)alkyl, (3-6C)cycloalkyl, or (3-6C)cycloalkyl(1-2C)alkyl;
and wherein any alkyl, alkoxy, cycloalkyl, cycloalkyl-alkyl, phenyl, heteroaryl or heterocyclyl group present in a R_{5c} group, or any alkyl, cycloalkyl or cycloalkyl-alkyl group present in a R_{5d} or R₅ₑ group, is optionally further substituted by one or more substituents independently selected from hydroxy, cyano, halo, R_{5f}, —OR_{5f}, —NR_{5f}R_{5g} and —C(O)—R_{5f}, wherein R_{5f} and R_{5g} are both independently selected from hydrogen, (1-4C)alkyl, hydroxy(1-4C)alkyl, halo(1-4C)alkyl, or (1-2C)alkoxy(1-4C)alkyl;
or R₄ and R₅, R₅ and R₆, or R₆ and R₇ are linked to form a fused phenyl, 5- or 6-membered heteroaryl or 5 to 7-membered heterocyclic ring, which is optionally substituted by one or more substituent groups selected from hydroxy, cyano, halo, (1-2C)alkyl, (1-2C)haloalkyl, (1-2C)alkoxy or (1-2C)haloalkoxy;
(ii) a group of the formula:

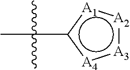

wherein:
A₁ is selected from N, NR_{1N}, O, S or CR₇;
A₂ is selected from N, NR_{1N}, O, S or CR₄;
A₂ is selected from N, NR_{1N}, O, S or CR₅;
A₄ is selected from N, NR_{1N}, O, S or CR₇;
R_{1N} is selected from hydrogen, (1-4C)alkyl, (2-4C)alkanoyl, (3-6C)cycloalkyl, (3-6C)cycloalkyl-(1-2C)alkyl, a carbon-linked 4 to 7 membered heterocyclyl, or 4 to 7 membered heterocyclyl(1-2C)alkyl, and wherein any alkyl, alkanoyl, cycloalkyl, cycloalkyl-alkyl, heterocyclyl, heterocyclyl-alkyl group is optionally substituted with one or more R_{5c} groups defined above;
R₄, R₅ and R₇ are as defined above;
and with the proviso that:
   (i) only one of A₁, A₂, A₃ and A₄ can be O or S;
   (ii) only one of A₁, A₂, A₃ and A₄ can be NR_{1N};
   (iii) 1 to 3 of A₁, A₂, A₃ and A₄ can be N;
(iii) a group of the formula:

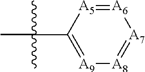

wherein:
A₅ is selected from N or CH;
A₆ is selected from N or CR₄;
A₇ is selected from N or CR₅;
A₈ is selected from N or CR₆;
A₉ is selected from N or CR₇;
R₄, R₅, R₆ and R₇ are as defined above;
and with the proviso that only one of A₅, A₆, A₇, A₈ and A₉ can be N;
(iv) a group of the formula:

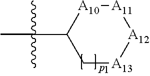

wherein:
p1 is 0 or 1;
A₁₀ is selected from CH₂ or CHR₇;
A₁₁ is selected from CH₂ or CHR₄;
A₁₂ is selected from NR_{5N}, CH₂ or CHR₅;
A₁₃ is selected from CH₂ or CHR₆;
R₄, R₅, R₆ and R₇ are as defined above;
R_{5N} is:
   a) hydrogen;
   b) a group of the formula:

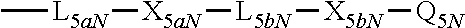

wherein:
L_{5aN} is absent or (1-30)alkylene;
X_{5aN} is absent or is selected from the group consisting of:
(i) —C(O)—, —S(O)₀₋₂—, —C(O)—N(RS₁)— when L_{5aN} is absent; or
(ii) —O—, —C(O)—, —NR₅ₐ₁, —C(O)—O—, —O—C(O)—, —S(O)O-2—, —C(O)—N(R₅ₐ₁)—, —N(R₅ₐ₁)—C(O)—, —O—C(O)—N(R₅ₐ₁)—, —N(R₅ₐ₁)—C(O)—O—, —N(R₅ₐ₂)—C(O)—NR₅ₐ₁—, —SO₂N(R₅ₐ₁)— or —N(R₅ₐ₁)SO₂— when L_{5aN} is present;
where R₅ₐ₁ and R₅ₐ₂ are as defined above;
L_{5bN} is absent or (1-2C)alkylene;
X_{5bN} is absent or is selected from the group consisting of —O—, —C(O)—, —NR_{5b1}—, —S(O)₀₋₂—, —C(O)—N(R_{5b1})—, —N(R_{5b1})—C(O)—, —SO₂N(R_{5b1})— or —N(R_{5b1})SO₂—, where R_{5b1} and R_{5b2} are independently selected from the group consisting of hydrogen or (1-2C)alkyl;
Q_{5N} is selected from the group consisting of:
(i) hydrogen;
(ii) (1-4C)alkyl, (3-6C)cycloalkyl, (3-6C)cycloalkyl(1-1C)alkyl, phenyl, phenyl(1-2C)alkyl, 5- or 6-membered heteroaryl, 5- or 6-membered heteroaryl(1-2C)alkyl;
and wherein Q_{5N} is optionally substituted with one or more R_{5c} groups as defined above;
and with the proviso that one or two of A₁₀, A₁₁, A₁₂ and A₁₃ can be N;
(v) a group of the formula:

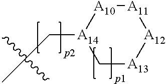

wherein:
p1, A₁₀, A₁₁, A₁₂, and A₁₃ are as defined above;
p2 is 1 to 4;
A₁₄ is C or N;
and with the proviso that one of A₁₀, A₁₁, A₁₂ and A₁₃ can be N.
(39) Q₁ is selected from hydrogen or:
   (i) a group of the formula:

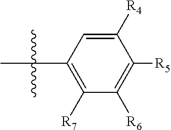

wherein:
R₄, R₆ and R₇ are each independently selected from hydrogen, or a group of the formula:

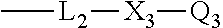

wherein:
L₂ is absent or (1-2C)alkylene;
X₃ is absent or is selected from the group consisting of —O—, —NR₄ₐ—, —C(O)—N(R₄ₐ)—, —N(R₄ₐ)—C(O)—, —SO₂N(R₄ₐ)— or —N(R₄ₐ)SO₂—, where R₄ₐ and R_{4b} are independently selected from the group consisting of hydrogen or methyl;
Q₃ is selected from the group consisting of hydrogen, (1-4C)alkyl, (3-6C)cycloalkyl, (3-6C)cycloalkyl(1-4C)alkyl, phenyl, 3 to 8-membered heterocyclyl, 5 or 6-membered heteroaryl or 5 or 6-membered heteroaryl(1-4C)alkyl wherein any alkyl, cycloalkyl, cycloalkylalkyl, phenyl, heterocyclyl or heteroaryl in Q₃ is optionally substituted with one or more R_{4c} groups; and
each R_{4c} group present is independently selected from the group consisting of hydroxy, cyano, oxo, halogen, methyl, methoxy, (3-6C)cycloalkyl, —NR_{4d}R₄ₑ, —C(O)—R_{4d} or —C(O)—OR_{4d},
wherein R_{4d} and R₄ₑ are each independently hydrogen or methyl; and wherein any alkyl, alkoxy, cycloalkyl, cycloalkyl-alkyl, phenyl, heteroaryl or heterocyclyl group present in a R_{4c} group, or any methyl group present in a R_{4d} or R₄ₑ group, is optionally further substituted by one or more substituents independently selected from hydroxy, cyano, halogen, (1-2C)alkyl, —OR_{4f}, —NR_{4f}R_{4g}, —S(O)₀₋₂—R_{4f} and —C(O)—R_{4f}, wherein R_{4f} and R_{4g} are both independently selected from hydrogen and (1-2C)alkyl;
R₅ is:
   a) hydrogen, halo, cyano; or
   b) a group of the formula:

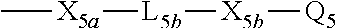

wherein:
X₅ₐ is absent or is selected from the group consisting of —O—, —C(O)—, —NR₅ₐ₁— or —N(R₅ₐ₁)—C(O)—, where R₅ₐ₁ is independently selected from the group consisting of hydrogen or methyl; L_{5b} is absent or (1-4C)alkylene;
X_{5b} is absent or is selected from the group consisting of —O—, —C(O)— or —NR_{5b1}—, where R_{5b}, and R_{5b2} are independently selected from the group consisting of hydrogen or (1-2C)alkyl;
Q₅ is selected from the group consisting of hydrogen, (1-6C)alkyl or 3 to 8-membered heterocyclyl and wherein Q₅ is optionally substituted with one or more R_{5c} groups;
and each R₅, group present is independently selected from the group consisting of hydroxy, cyano, halogen, methyl, methoxy
(ii) a group of the formula:

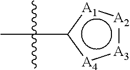

wherein:
A₁ is selected from N, NR_{1N}, O, S or CR₇;
A₂ is selected from N, NR_{1N}, O, S or CR₄;
A₃ is selected from N, NR_{1N}, O, S or CR₅;
A₄ is selected from N, NR_{1N}, O, S or CR₇;
R_{1N} is selected from hydrogen, (1-4C)alkyl, (2-4C)alkanoyl, (3-6C)cycloalkyl, (3-6C)cycloalkyl-(1-2C)alkyl, a carbon-linked 4 to 7 membered heterocyclyl, or 4 to 7 membered heterocyclyl(1-2C)alkyl, and wherein any alkyl, alkanoyl, cycloalkyl, cycloalkyl-alkyl, heterocyclyl, heterocyclyl-alkyl group is optionally substituted with one or more R_{5c} groups defined above;
R₄, R₅ and R₇ are as defined above;
and with the proviso that:
   (i) only one of A₁, A₂, A₃ and A₄ can be O or S;
   (ii) only one of A₁, A₂, A₃ and A₄ can be NR_{1N};
   (iii) 1 to 3 of A₁, A₂, A₃ and A₄ can be N;
(iii) a group of the formula:

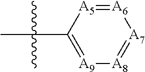

wherein:
A₅ is selected from N or CH;
As is selected from N or CR₄;
A₇ is selected from N or CR₅;
A₉ is selected from N or CR₅;
A₉ is selected from N or CR₇;
R₄, R₅, R₆ and R₇ are as defined above;
and with the proviso that only one of A₅, A₆, A₇, A₈ and A₉ can be N;
(iv) a group of the formula:

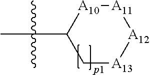

wherein:
p1 is 0 or 1;
A₁₀ is selected from CH₂ or CHR₇;
A₁₁ is selected from CH₂ or CHR₄;
A₁₂ is selected from NR_{5N}, CH₂ or CHR₅;
A₁₃ is selected from CH₂ or CHR₆;
R₄, R₅, R₆ and R₇ are as defined above;
R_{5N} is
   a) hydrogen;
   b) a group of the formula:

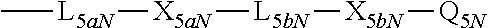

wherein:
L_{5aN} is absent or (1-3C)alkylene;
X_{5aN} is absent or is selected from the group consisting of:
(i) —C(O)—, —S(O)₀₋₂— or —C(O)—N(R₅ₐ₁)— when L_{5aN} is absent; or
(ii) —O—, —C(O)—, —NR₅ₐ₁—, —C(O)—O—, —O—C(O)—, —S(O)₀₋₂—, —C(O)—N(R₅ₐ₁)—, —N(R₅ₐ₁)—C(O)—, —O—C(O)—N(R₅ₐ₁)—, —N(R₅ₐ₁)—C(O)—O— or —N(R₅ₐ₂)—C(O)—NR₅ₐ₁— when L_{5aN} is present;
where R₅ₐ₁ and R₅ₐ₂ are as defined above;
L_{5bN} is absent or (1-2C)alkylene;
X_{5bN} is absent or is selected from the group consisting of —O—, —C(O)—, —NRSb—, —S(O)₀₋₂—, —C(O)—N(R_{5b})— or —N(R_{5b})—C(O)—, where R_{5b}, and R_{5b2} are independently selected from the group consisting of hydrogen or (1-2C)alkyl;
Q_{5N} is selected from the group consisting of:
(i) hydrogen;
(ii) (1-4C)alkyl or (3-6C)cycloalkyl;
and wherein Q_{5N} is optionally substituted with one or more R_{5c} groups as defined above;
and with the proviso that one or two of A₁₀, A₁₁, A₁₂ and A₁₃ can be N;
(v) a group of the formula:

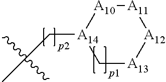

wherein:
p1, A₁₀, A₁₁, A₁₂, and A₁₃ are as defined above;
p2 is 1 to 4;
A₁₄ is C or N;
and with the proviso that one of A₁₀, A₁₁, A₁₂ and A₁₃ can be N.
(40) Q₁ is selected from hydrogen or:
   (i) a group of the formula:

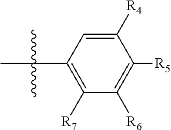

wherein:
two of R₄, R₆ and R₇ are hydrogen, and the other is hydrogen or a group of the formula:

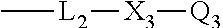

wherein:
L₂ is absent or (1-2C)alkylene;
X₃ is absent or is selected from the group consisting of —O—, —NR₄ₐ—, —C(O)—N(R₄ₐ)—, —N(R₄ₐ)—C(O)—, —SO₂N(R₄ₐ)— or —N(R₄ₐ)SO₂—, where R₄ₐ and R_{4b} are independently selected from the group consisting of hydrogen or methyl;
Q₃ is selected from the group consisting of hydrogen, (1-4C)alkyl, (3-6C)cycloalkyl, (3-6C)cycloalkyl(1-4C)alkyl, phenyl, 3 to 8-membered heterocyclyl, 5 or 6-membered heteroaryl or 5 or 6-membered heteroaryl(1-4C)alkyl wherein any alkyl, cycloalkyl, cycloalkylalkyl, phenyl, heterocyclyl or heteroaryl in Q₃ is optionally substituted with one or more R₄ₑ groups; and
each R_{4c} group present is independently selected from the group consisting of hydroxy, cyano, oxo, halogen, methyl, methoxy, (3-6C)cycloalkyl, —NR_{4d}R₄ₑ, —C(O)—R_{4d} or —C(O)—OR_{4d},
wherein R_{4d} and R₄ₑ are each independently hydrogen or methyl;
and wherein any methyl, methoxy or cycloalkyl, group present in a R₄ₑ group, or any methyl group present in a R_{4d} or R₄ₑ group, is optionally further substituted by one or more substituents independently selected from hydroxy, cyano, halogen, —OR_{4f}, —NR_{4f}R_{4g}, —S(O)₀₋₂—R₄ᵣ and —C(O)—R_{4f}, wherein R_{4f} and R_{4g} are both independently selected from hydrogen and (1-2C)alkyl;
R₅ is hydrogen or a group of the formula:

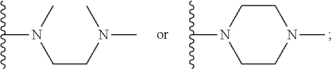

and
(ii) a group of the formula:

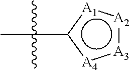

wherein:
A₁ is selected from N, NR_{1N}, O, S or CR₇;
A₂ is selected from N, NR_{1N}, O, S or CR₄;
A₃ is selected from N, NR_{1N}, O, S or CR₅;
A₄ is selected from N, NR_{1N}, O, S or CR₇;
R_{1N} is selected from hydrogen, (1-4C)alkyl, (2-4C)alkanoyl, (3-6C)cycloalkyl, (3-6C)cycloalkyl-(1-2C)alkyl, a carbon-linked 4 to 7 membered heterocyclyl, or 4 to 7 membered heterocyclyl(1-2C)alkyl, and wherein any alkyl, alkanoyl, cycloalkyl, cycloalkyl-alkyl, heterocyclyl, heterocyclyl-alkyl group is optionally substituted with one or more R_{5c} groups defined above; R₄, R₅ and R₇ are as defined above;
and with the proviso that:
   (i) only one of A₁, A₂, A₃ and A₄ can be O or S;
   (ii) only one of A₁, A₂, A₃ and A₄ can be NR_{1N};
   (iii) 1 to 3 of A₁, A₂, A₃ and A₄ can be N;
(iii) a group of the formula:

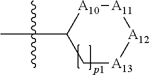

wherein:
p1 is 0 or 1;
A₁₀ is selected from CH₂ or CHR₇;
A₁₁ is selected from CH₂ or CHR₄;
A₁₂ is selected from NR_{5N}, CH₂ or CHR₅;
A₁₃ is selected from CH₂ or CHR₆;
R₄, R₅, R₆ and R₇ are as defined above;
R_{5N} is:
   a) hydrogen;
   b) a group of the formula:

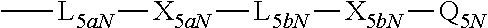

wherein:
L_{5aN} is absent or (1-3C)alkylene;
X_{5aN} is absent or is selected from the group consisting of:
(i) —C(O)—, —S(O)₀₋₂— or —C(O)—N(R₅ₐ₁)— when L_{5aN} is absent; or
(ii) —O—, —C(O)—, —NR₅ₐ₁—, —C(O)—O—, —O—C(O)—, —S(O)O-2—, —C(O)—N(R₅ₐ₁)—, —N(R₅ₐ₁)—C(O)—, —O—C(O)—N(R₅ₐ₁)—, —N(R₅ₐ₁)—C(O)—O— or —N(R₅ₐ₂)—C(O)—NR₅ₐ₁— when L_{5aN} is present;
where R₅ₐ₁ and R₅ₐ₂ are as defined above;
L_{5bN} is absent or (1-2C)alkylene;
X_{5bN} is absent or is selected from the group consisting of —O—, —C(O)—, —NR_{5b1}—, —S(O)₀₋₂—, —C(O)—N(R_{5b1})— or —N(R_{5b1})—C(O)—, where R_{5b}, and R_{5b2} are independently selected from the group consisting of hydrogen or (1-2C)alkyl;
Q_{5N} is selected from the group consisting of:
(i) hydrogen;
(ii) (1-4C)alkyl or (3-6C)cycloalkyl;
and wherein Q_{5N} is optionally substituted with one or more R_{5c} groups as defined above;
and with the proviso that one or two of A₁₀, A₁₁, A₁₂ and A₁₃ can be N;
(iv) a group of the formula:

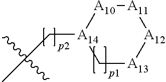

wherein:
p1, A₁₀, A₁₁, A₁₂, and A₁₃ are as defined above;
p2 is 1 to 4;
A₁₄ is C or N;
and with the proviso that one of A₁₀, A₁₁, A₁₂ and A₁₃ can be N.

Suitably, in any of the definitions of formula I set out herein, a heteroaryl is a 5- or 6-membered heteroaryl ring comprising one, two or three heteroatoms selected from N, O or S.

More suitably, in any of the definitions of formula I set out herein, a heteroaryl is a 5- or 6-membered heteroaryl ring comprising one or two N atoms.

Suitably, in any of the definitions of formula I set out herein, a heterocyclyl group is a 4-, 5-, 6- or 7-membered heterocyclyl ring comprising one, two or three heteroatoms selected from N, O or S. Most suitably, a heterocyclyl group is a 4-, 5- or 6-membered ring comprising one or two heteroatoms selected from N, O or S [e.g. morpholinyl (e.g. 4-morpholinyl), piperidinyl, piperazinyl or pyrrolidinyl].

Suitably, in any of the definitions of formula I set out herein, R₁ is as defined in formula I above or is as defined in any one of paragraphs (1), (2) or (3) above. In a particular group of compounds of the invention, R₁ is as defined in paragraph (1) above. In another particular group of compounds of the invention, R₁ is as defined in paragraph (2) above. In another particular group of compounds of the invention, R₁ is as defined in paragraph (3) above.

Suitably, in any of the definitions of formula I set out herein, R₂ is as defined in formula I above or is as defined in any one of paragraphs (4), (5) or (6) above. In a particular group of compounds of the invention, R₂ is as defined in paragraph (4) above. In another particular group of compounds of the invention, R₂ is as defined in paragraph (5) above. In another particular group of compounds of the invention, R₂ is as defined in paragraph (6) above.

Suitably, in any of the definitions of formula I set out herein, R₃ is as defined in formula I above or is as defined in any one of paragraphs (7), (8), (9), (10), (11), (12), (13) or (14) above. In a particular group of compounds of the invention, R₃ is as defined in paragraph (7) above. In another particular group of compounds of the invention, R₃ is as defined in paragraph (8) above. In another particular group of compounds of the invention, R₃ is as defined in paragraph (9) above. In a particular group of compounds of the invention, R₃ is as defined in paragraph (10) above. In another particular group of compounds of the invention, R₃ is as defined in paragraph (11) above. In another particular group of compounds of the invention, R₃ is as defined in paragraph (12) above. In another particular group of compounds of the invention, R₃ is as defined in paragraph (13) above. In another particular group of compounds of the invention, R₃ is as defined in paragraph (14) above.

More suitably, in any of the definitions of formula I set out herein, R₃ is as defined in formula I above or is as defined in any one of paragraphs (21), (22), (23), (24), (25), (26), (27), (28), (29), (30), (31), (32), (33), (34) or (35) above. In a particular group of compounds of the invention, R₃ is as defined in paragraph (21) above. In another particular group of compounds of the invention, R₃ is as defined in paragraph (22) above. In another particular group of compounds of the invention, R₃ is as defined in paragraph (23) above. In a particular group of compounds of the invention, R₃ is as defined in paragraph (24) above. In another particular group of compounds of the invention, R₃ is as defined in paragraph (25) above. In another particular group of compounds of the invention, R₃ is as defined in paragraph (26) above. In another particular group of compounds of the invention, R₃ is as defined in paragraph (27) above. In another particular group of compounds of the invention, R₃ is as defined in paragraph (28) above. In another particular group of compounds of the invention, R₃ is as defined in paragraph (29) above. In another particular group of compounds of the invention, R₃ is as defined in paragraph (30) above. In another particular group of compounds of the invention, R₃ is as defined in paragraph (31) above. In another particular group of compounds of the invention, R₃ is as defined in paragraph (32) above. In another particular group of compounds of the invention, R₃ is as defined in paragraph (33) above. In another particular group of compounds of the invention, R₃ is as defined in paragraph (34) above. In another particular group of compounds of the invention, R₃ is as defined in paragraph (35) above.

Suitably, in any of the definitions of formula I set out herein, Q₁ is as defined in formula I above or is as defined in any one of paragraphs (15), (16), (17), (18), (19) or (20) above. In a particular group of compounds of the invention, Q, is as defined in paragraph (15) above.

In another particular group of compounds of the invention, Q, is as defined in paragraph (16) above. In another particular group of compounds of the invention, Q, is as defined in paragraph (17) above. In another particular group of compounds of the invention, Q₁ is as defined in paragraph (18) above. In another particular group of compounds of the invention, Q₁ is as defined in paragraph (19) above. In another particular group of compounds of the invention, Q, is as defined in paragraph (20) above.

Suitably, in any of the definitions of formula I set out herein, Q, may be as defined in formula I above or is as defined in any one of paragraphs (36), (37), (38), (39), or (40) above. In a particular group of compounds of the invention, Q, is as defined in paragraph (36) above. In another particular group of compounds of the invention, Q, is as defined in paragraph (37) above. In another particular group of compounds of the invention, Q, is as defined in paragraph (38) above. In another particular group of compounds of the invention, Q₁ is as defined in paragraph (39) above. In another particular group of compounds of the invention, Q₁ is as defined in paragraph (40) above.

In a particular group of compounds of formula I defined herein:
R₁ is as defined in formula I above;
R₂ is as defined in paragraph (4) above;
R₃ is as defined in any one of paragraphs (7), (8), (9), (10), (11), (12), (13) or (14) above;
Q₁ is as defined in paragraph (15) above.

In a particular group of compounds of formula I defined herein:
R₁ is as defined in formula I above;
R₂ is as defined in paragraph (4) above;
R₃ is as defined in any one of paragraphs (21), (22), (23), (24), (25), (26), (27), (28), (29), (30), (31), (32), (33), (34) or (35) above;
Q₁ is as defined in paragraph (15) above.

In a particular group of compounds of formula I defined herein:
R₁ is as defined in formula I above;
R₂ is as defined in paragraph (4) above;
R₃ is as defined in any one of paragraphs (10), (11), (12), (13) or (14) above;
Q₁ is as defined in paragraph (16) above.

In a particular group of compounds of formula I defined herein:
R₁ is as defined in formula I above;
R₂ is as defined in paragraph (4) above;
R₃ is as defined in any one of paragraphs (21), (22), (23), (24), (25), (26), (27), (28), (29), (30), (31), (32), (33), (34) or (35) above;
Q₁ is as defined in paragraph (16) above.

In a particular group of compounds of formula I defined herein:
R₁ is as defined in formula I above;
R₂ is as defined in paragraph (4) above;
R₃ is as defined in any one of paragraphs (10), (11), (12), (13) or (14) above;
Q₁ is as defined in paragraph (17) above.

In a particular group of compounds of formula I defined herein:
R₁ is as defined in formula I above;
R₂ is as defined in paragraph (4) above;
R₃ is as defined in any one of paragraphs (21), (22), (23), (24), (25), (26), (27), (28), (29), (30), (31), (32), (33), (34) or (35) above;
Q₁ is as defined in paragraph (17) above.

In a particular group of compounds of formula I defined herein:
R₁ is as defined in formula I above;
R₂ is as defined in paragraph (4) above;
R₃ is as defined in any one of paragraphs (10), (11), (12), (13) or (14) above;
Q₁ is as defined in paragraph (18) above.

In a particular group of compounds of formula I defined herein:
R₁ is as defined in formula I above;
R₂ is as defined in paragraph (4) above;
R₃ is as defined in any one of (21), (22), (23), (24), (25), (26), (27), (28), (29), (30), (31), (32), (33), (34) or (35) above;
Q₁ is as defined in paragraph (18) above.

In a particular group of compounds of formula I defined herein:
R₁ is as defined in formula I above;
R₂ is as defined in paragraph (4) above;
R₃ is as defined in any one of paragraphs (10), (11), (12), (13) or (14) above;
Q₁ is as defined in paragraph (19) above.

In a particular group of compounds of formula I defined herein:
R₁ is as defined in formula I above;
R₂ is as defined in paragraph (4) above;
R₃ is as defined in any one of paragraphs (21), (22), (23), (24), (25), (26), (27), (28), (29), (30), (31), (32), (33), (34) or (35) above;
Q₁ is as defined in paragraph (19) above.

In a particular group of compounds of formula I defined herein:
R₁ is as defined in formula I above;
R₂ is as defined in paragraph (4) above;
R₃ is as defined in any one of paragraphs (10), (11), (12), (13) or (14) above;
Q₁ is as defined in paragraph (20) above.

In a particular group of compounds of formula I defined herein:
R₁ is as defined in formula I above;
R₂ is as defined in paragraph (4) above;
R₃ is as defined in any one of paragraphs (21), (22), (23), (24), (25), (26), (27), (28), (29), (30), (31), (32), (33), (34) or (35) above;
Q₁ is as defined in paragraph (20) above.

In a particular group of compounds of formula I defined herein:
R₁ is as defined in formula I above;
R₂ is as defined in paragraph (5) above;
R₃ is as defined in any one of paragraphs (7), (8), (9), (10), (11), (12), (13) or (14) above;
Q₁ is as defined in paragraph (15) above.

In a particular group of compounds of formula I defined herein:
R₁ is as defined in formula I above;
R₂ is as defined in paragraph (5) above;
R₃ is as defined in any one of paragraphs (21), (22), (23), (24), (25), (26), (27), (28), (29), (30), (31), (32), (33), (34) or (35) above;
Q₁ is as defined in paragraph (15) above.

In a particular group of compounds of formula I defined herein:
R₁ is as defined in formula I above;
R₂ is as defined in paragraph (5) above;
R₃ is as defined in any one of paragraphs (10), (11), (12), (13) or (14) above;
Q₁ is as defined in paragraph (16) above.

In a particular group of compounds of formula I defined herein:
R₁ is as defined in formula I above;
R₂ is as defined in paragraph (5) above;
R₃ is as defined in any one of paragraphs (21), (22), (23), (24), (25), (26), (27), (28), (29), (30), (31), (32), (33), (34) or (35) above;
Q₁ is as defined in paragraph (16) above.

In a particular group of compounds of formula I defined herein:
R₁ is as defined in formula I above;
R₂ is as defined in paragraph (5) above;
R₃ is as defined in any one of paragraphs (10), (11), (12), (13) or (14) above;
Q₁ is as defined in paragraph (17) above.

In a particular group of compounds of formula I defined herein:
R₁ is as defined in formula I above;
R₂ is as defined in paragraph (5) above;
R₃ is as defined in any one of paragraphs (21), (22), (23), (24), (25), (26), (27), (28), (29), (30), (31), (32), (33), (34) or (35) above;
Q₁ is as defined in paragraph (17) above.

In a particular group of compounds of formula I defined herein:
R₁ is as defined in formula I above;
R₂ is as defined in paragraph (5) above;
R₃ is as defined in any one of paragraphs (10), (11), (12), (13) or (14) above;
Q₁ is as defined in paragraph (18) above.

In a particular group of compounds of formula I defined herein:
R₁ is as defined in formula I above;
R₂ is as defined in paragraph (5) above;
R₃ is as defined in any one of paragraphs (21), (22), (23), (24), (25), (26), (27), (28), (29), (30), (31), (32), (33), (34) or (35) above;
Q₁ is as defined in paragraph (18) above.

In a particular group of compounds of formula I defined herein:
R₁ is as defined in formula I above;
R₂ is as defined in paragraph (5) above;
R₃ is as defined in any one of paragraphs (10), (11), (12), (13) or (14) above;
Q₁ is as defined in paragraph (19) above.

In a particular group of compounds of formula I defined herein:
R₁ is as defined in formula I above;
R₂ is as defined in paragraph (5) above;
R₃ is as defined in any one of paragraphs (21), (22), (23), (24), (25), (26), (27), (28), (29), (30), (31), (32), (33), (34) or (35) above;
Q₁ is as defined in paragraph (19) above.

In a particular group of compounds of formula I defined herein:
R₁ is as defined in formula I above;
R₂ is as defined in paragraph (5) above;
R₃ is as defined in any one of paragraphs (10), (11), (12), (13) or (14) above;
Q₁ is as defined in paragraph (20) above.

In a particular group of compounds of formula I defined herein:
R₁ is as defined in formula I above;
R₂ is as defined in paragraph (5) above;
R₃ is as defined in any one of paragraphs (21), (22), (23), (24), (25), (26), (27), (28), (29), (30), (31), (32), (33), (34) or (35) above;
Q₁ is as defined in paragraph (20) above.

In a particular group of compounds of formula I defined herein:
R₁ is as defined in formula I above;
R₂ is as defined in paragraph (4) above;
R₃ is as defined in any one of paragraphs (7), (8), (9), (10), (11), (12), (13) or (14) above;
Q₁ is as defined in paragraph (36) above.

In a particular group of compounds of formula I defined herein:
R₁ is as defined in formula I above;
R₂ is as defined in paragraph (4) above;
R₃ is as defined in any one of paragraphs (21), (22), (23), (24), (25), (26), (27), (28), (29), (30), (31), (32), (33), (34) or (35) above;
Q₁ is as defined in paragraph (36) above.

In a particular group of compounds of formula I defined herein:
R₁ is as defined in formula I above;
R₂ is as defined in paragraph (4) above;
R₃ is as defined in any one of paragraphs (10), (11), (12), (13) or (14) above;
Q₁ is as defined in paragraph (36) above.

In a particular group of compounds of formula I defined herein:
R₁ is as defined in formula I above;
R₂ is as defined in paragraph (4) above;
R₃ is as defined in any one of paragraphs (21), (22), (23), (24), (25), (26), (27), (28), (29), (30), (31), (32), (33), (34) or (35) above;
Q₁ is as defined in paragraph (36) above.

In a particular group of compounds of formula I defined herein:
R₁ is as defined in formula I above;
R₂ is as defined in paragraph (4) above;
R₃ is as defined in any one of paragraphs (10), (11), (12), (13) or (14) above;
Q₁ is as defined in paragraph (37) above.

In a particular group of compounds of formula I defined herein:
R₁ is as defined in formula I above;
R₂ is as defined in paragraph (4) above;
R₃ is as defined in any one of paragraphs (21), (22), (23), (24), (25), (26), (27), (28), (29), (30), (31), (32), (33), (34) or (35) above;
Q₁ is as defined in paragraph (37) above.

In a particular group of compounds of formula I defined herein:
R₁ is as defined in formula I above;
R₂ is as defined in paragraph (4) above;
R₃ is as defined in any one of paragraphs (10), (11), (12), (13) or (14) above;
Q₁ is as defined in paragraph (38) above.

In a particular group of compounds of formula I defined herein:
R₁ is as defined in formula I above;
R₂ is as defined in paragraph (4) above;
R₃ is as defined in any one of (21), (22), (23), (24), (25), (26), (27), (28), (29), (30), (31), (32), (33), (34) or (35) above;
Q₁ is as defined in paragraph (38) above.

In a particular group of compounds of formula I defined herein:
R₁ is as defined in formula I above;
R₂ is as defined in paragraph (4) above;
R₃ is as defined in any one of paragraphs (10), (11), (12), (13) or (14) above;
Q₁ is as defined in paragraph (39) above.

In a particular group of compounds of formula I defined herein:
R₁ is as defined in formula I above;
R₂ is as defined in paragraph (4) above;
R₃ is as defined in any one of paragraphs (21), (22), (23), (24), (25), (26), (27), (28), (29), (30), (31), (32), (33), (34) or (35) above;
Q₁ is as defined in paragraph (39) above.

In a particular group of compounds of formula I defined herein:
R₁ is as defined in formula I above;
R₂ is as defined in paragraph (4) above;
R₃ is as defined in any one of paragraphs (10), (11), (12), (13) or (14) above;
Q₁ is as defined in paragraph (40) above.

In a particular group of compounds of formula I defined herein:
R₁ is as defined in formula I above;
R₂ is as defined in paragraph (4) above;
R₃ is as defined in any one of paragraphs (21), (22), (23), (24), (25), (26), (27), (28), (29), (30), (31), (32), (33), (34) or (35) above;
Q₁ is as defined in paragraph (40) above.

In a particular group of compounds of formula I defined herein:
R₁ is as defined in formula I above;
R₂ is as defined in paragraph (5) above;
R₃ is as defined in any one of paragraphs (7), (8), (9), (10), (11), (12), (13) or (14) above;
Q₁ is as defined in paragraph (36) above.

In a particular group of compounds of formula I defined herein:
R₁ is as defined in formula I above;
R₂ is as defined in paragraph (5) above;
R₃ is as defined in any one of paragraphs (21), (22), (23), (24), (25), (26), (27), (28), (29), (30), (31), (32), (33), (34) or (35) above;
Q₁ is as defined in paragraph (36) above.

In a particular group of compounds of formula I defined herein:
R₁ is as defined in formula I above;
R₂ is as defined in paragraph (5) above;
R₃ is as defined in any one of paragraphs (10), (11), (12), (13) or (14) above;
Q₁ is as defined in paragraph (36) above.

In a particular group of compounds of formula I defined herein:
R₁ is as defined in formula I above;
R₂ is as defined in paragraph (5) above;
R₃ is as defined in any one of paragraphs (21), (22), (23), (24), (25), (26), (27), (28), (29), (30), (31), (32), (33), (34) or (35) above;
Q₁ is as defined in paragraph (36) above.

In a particular group of compounds of formula I defined herein:
R₁ is as defined in formula I above;
R₂ is as defined in paragraph (5) above;
R₃ is as defined in any one of paragraphs (10), (11), (12), (13) or (14) above;
Q₁ is as defined in paragraph (37) above.

In a particular group of compounds of formula I defined herein:
R₁ is as defined in formula I above;
R₂ is as defined in paragraph (5) above;
R₃ is as defined in any one of paragraphs (21), (22), (23), (24), (25), (26), (27), (28), (29), (30), (31), (32), (33), (34) or (35) above;
Q₁ is as defined in paragraph (37) above.

In a particular group of compounds of formula I defined herein:
R₁ is as defined in formula I above;
R₂ is as defined in paragraph (5) above;
R₃ is as defined in any one of paragraphs (10), (11), (12), (13) or (14) above;
Q₁ is as defined in paragraph (38) above.

In a particular group of compounds of formula I defined herein:
R₁ is as defined in formula I above;
R₂ is as defined in paragraph (5) above;
R₃ is as defined in any one of paragraphs (21), (22), (23), (24), (25), (26), (27), (28), (29), (30), (31), (32), (33), (34) or (35) above;
Q₁ is as defined in paragraph (38) above.

In a particular group of compounds of formula I defined herein:
R₁ is as defined in formula I above;
R₂ is as defined in paragraph (5) above;
R₃ is as defined in any one of paragraphs (10), (11), (12), (13) or (14) above;
Q₁ is as defined in paragraph (39) above.

In a particular group of compounds of formula I defined herein:
R₁ is as defined in formula I above;
R₂ is as defined in paragraph (5) above;
R₃ is as defined in any one of paragraphs (21), (22), (23), (24), (25), (26), (27), (28), (29), (30), (31), (32), (33), (34) or (35) above;
Q₁ is as defined in paragraph (39) above.

In a particular group of compounds of formula I defined herein:
R₁ is as defined in formula I above;
R₂ is as defined in paragraph (5) above;
R₃ is as defined in any one of paragraphs (10), (11), (12), (13) or (14) above;
Q₁ is as defined in paragraph (40) above.

In a particular group of compounds of formula I defined herein:
R₁ is as defined in formula I above;
R₂ is as defined in paragraph (5) above;
R₃ is as defined in any one of paragraphs (21), (22), (23), (24), (25), (26), (27), (28), (29), (30), (31), (32), (33), (34) or (35) above;
Q₁ is as defined in paragraph (40) above.

In a particular group of compounds of the invention, the compound is a compound of formula I defined herein in which the compound has the formula Ia shown below, or a pharmaceutically acceptable salt thereof:

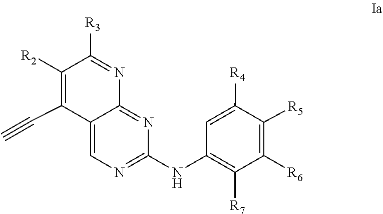

wherein R₂, R₃, R₄, R₅, R₆ and R₇ each have any one of the definitions set out herein.

Suitably, in any of the definitions of formula Ia set out herein, R₂ is as defined in formula I above or is as defined in any one of paragraphs (4), (5) or (6) above. In a particular group of compounds of formula Ia, R₂ is as defined in paragraph (4) above. In another particular group of compounds of formula Ia, R₂ is as defined in paragraph (5) above. In another particular group of compounds of formula Ia, R₂ is as defined in paragraph (6) above.

Suitably, in any of the definitions of formula Ia set out herein, R₃ is as defined in formula I above or is as defined in any one of paragraphs (7), (8), (9), (10), (11), (12), (13) or (14) above. In a particular group of compounds of formula Ia, R₃ is as defined in paragraph (7) above. In another particular group of compounds of formula Ia, R₃ is as defined in paragraph (8) above. In another particular group of compounds of formula Ia, R₃ is as defined in paragraph (9) above. In a particular group of compounds of formula Ia, R₃ is as defined in paragraph (10) above. In another particular group of compounds of formula Ia, R₃ is as defined in paragraph (11) above. In another particular group of compounds of formula Ia, R₃ is as defined in paragraph (12) above. In another particular group of compounds of formula Ia, R₃ is as defined in paragraph (13) above. In another particular group of compounds of formula 1a, R₃ is as defined in paragraph (14) above.

More suitably, in any of the definitions of formula Ia set out herein, R₃ is as defined in formula I above or is as defined in any one of paragraphs (21), (22), (23), (24), (25), (26), (27), (28), (29), (30), (31), (32), (33), (34) or (35) above. In a particular group of compounds of formula Ia, R₃ is as defined in paragraph (21) above. In another particular group of compounds of formula Ia, R₃ is as defined in paragraph (22) above. In another particular group of compounds of formula Ia, R₃ is as defined in paragraph (23) above. In a particular group of compounds of formula Ia, R is as defined in paragraph (24) above. In another particular group of compounds of formula Ia, R₃ is as defined in paragraph (25) above. In another particular group of compounds of formula Ia, R₃ is as defined in paragraph (26) above. In another particular group of compounds of formula Ia, R₃ is as defined in paragraph (27) above.

In another particular group of compounds of formula Ia, R₃ is as defined in paragraph (28) above. In another particular group of compounds of formula Ia, R₃ is as defined in paragraph (29) above. In another particular group of compounds of formula Ia, R₃ is as defined in paragraph (30) above. In another particular group of compounds of formula Ia, R₃ is as defined in paragraph (31) above. In another particular group of compounds of formula Ia, R₃ is as defined in paragraph (32) above. In another particular group of compounds of formula Ia, R₃ is as defined in paragraph (33) above. In another particular group of compounds of formula 1a, R₃ is as defined in paragraph (34) above. In another particular group of compounds of formula Ia, R₃ is as defined in paragraph (35) above.

Suitably, in any of the definitions of formula Ia set out herein, R₄ and R₆ are as defined for formula I above or are as defined in any one of paragraphs (15), (16), (17), (18), (19) or (20) above. In a particular group of compounds of formula Ia, R₄ and R₆ are as defined in paragraph (15) above. In another particular group of compounds of formula Ia, R₄ and R₆ are as defined in paragraph (16) above. In another particular group of compounds of formula Ia, R₄ and R₆ are as defined in paragraph (17) above. In another particular group of compounds of formula Ia, R₄ and R₅ are as defined in paragraph (18) above. In another particular group of compounds of formula Ia, R₄ and R₆ are as defined in paragraph (19) above. In another particular group of compounds of formula Ia, R₄ and R₅ are as defined in paragraph (20) above.

Suitably, one of R₄ and R₆ is hydrogen and the other is as defined in any one of paragraphs (15), (16), (17), (18), (19) or (20) above.

Suitably, in any of the definitions of formula Ia set out herein, R₄ and R₆ are as defined for formula I above or are as defined in any one of paragraphs (36), (37), (38), (39) or (40) above. In another particular group of compounds of formula Ia, R₄ and R₆ are as defined in paragraph (36) above. In another particular group of compounds of formula Ia, R₄ and R₆ are as defined in paragraph (37) above. In another particular group of compounds of formula Ia, R₄ and R₆ are as defined in paragraph (38) above. In another particular group of compounds of formula Ia, R₄ and R₆ are as defined in paragraph (39) above. In another particular group of compounds of formula Ia, R₄ and R₆ are as defined in paragraph (40) above.

Suitably, one of R₄ and R₆ is hydrogen and the other is as defined in any one of paragraphs (36), (37), (38), (39) or (40) above.

Suitably, in any of the definitions of formula Ia set out herein, R₅ is as defined for formula I above or are as defined in any one of paragraphs (15), (16), (17), (18), (19) or (20) above. In a particular group of compounds of formula Ia, R₅ is as defined in paragraph (15) above. In another particular group of compounds of formula Ia, R₅ is as defined in paragraph (16) above. In another particular group of compounds of formula Ia, R₅ is as defined in paragraph (17) above. In another particular group of compounds of formula Ia, R₅ is as defined in paragraph (18) above. In another particular group of compounds of formula Ia, R₅ is as defined in paragraph (19) above. In another particular group of compounds of formula Ia, R₅ is as defined in paragraph (20) above.

Suitably, in any of the definitions of formula Ia set out herein, R₇ is as defined for formula I above or are as defined in any one of paragraphs (15), (16), (17), (18), (19) or (20) above. In a particular group of compounds of formula Ia, R₇ is as defined in paragraph (15) above. In another particular group of compounds of formula Ia, R₇ is as defined in paragraph (16) above. In another particular group of compounds of formula Ia, R₇ is as defined in paragraph (17) above. In another particular group of compounds of formula Ia, R₇ is as defined in paragraph (18) above. In another particular group of compounds of formula Ia, R₇ is as defined in paragraph (19) above. In another particular group of compounds of formula Ia, R₇ is as defined in paragraph (20) above.

Suitably, in any of the definitions of formula Ia set out herein, R₇ is as defined for formula I above or are as defined in any one of paragraphs (36), (37), (38), (39) or (40) above.

In another particular group of compounds of formula Ia, R₇ is as defined in paragraph (36) above. In another particular group of compounds of formula Ia, R₇ is as defined in paragraph (37) above. In another particular group of compounds of formula Ia, R₇ is as defined in paragraph (38) above. In another particular group of compounds of formula Ia, R₇ is as defined in paragraph (39) above. In another particular group of compounds of formula Ia, R₇ is as defined in paragraph (40) above.

In a particular group of compounds of formula Ia defined herein:
R₂ is as defined in paragraph (4) above;
R₃ is as defined in any one of paragraphs (7), (8), (9), (10), (11), (12), (13) or (14) above;
R₄, R₅, R₆ and R₇ are each independently as defined in paragraph (15) above.

In a particular group of compounds of formula Ia defined herein:
R₂ is as defined in paragraph (4) above;
R₃ is as defined in any one of paragraphs (10), (11), (12), (13) or (14) above;
Ra, R₅, R₆ and R₇ are each independently as defined in paragraph (16) above.

In a particular group of compounds of formula Ia defined herein:
R₂ is as defined in paragraph (4) above;
R₃ is as defined in any one of paragraphs (10), (11), (12), (13) or (14) above;
R₄, R₅, R₆ and R₇ are each independently as defined in paragraph (17) above.

In a particular group of compounds of formula Ia defined herein:
R₂ is as defined in paragraph (4) above;
R₃ is as defined in any one of paragraphs (10), (11), (12), (13) or (14) above;
R₄, R₅, R₆ and R₇ are each independently as defined in paragraph (18) above.

In a particular group of compounds of formula Ia defined herein:
R₂ is as defined in paragraph (4) above;
R₃ is as defined in any one of paragraphs (10), (11), (12), (13) or (14) above;
R₄, R₅, R₆ and R₇ are each independently as defined in paragraph (19) above.

In a particular group of compounds of formula Ia defined herein:
R₂ is as defined in paragraph (4) above;
R₃ is as defined in any one of paragraphs (10), (11), (12), (13) or (14) above;
R₄, R₅, R₆ and R₇ are each independently as defined in paragraph (20) above.

In a particular group of compounds of formula Ia defined herein:
R₂ is as defined in paragraph (5) above;
R₃ is as defined in any one of paragraphs (7), (8), (9), (10), (11), (12), (13) or (14) above;
R₄, R₅, R₆ and R₇ are each independently as defined in paragraph (15) above.

In a particular group of compounds of formula Ia defined herein:
R₂ is as defined in paragraph (5) above;
R₃ is as defined in any one of paragraphs (10), (11), (12), (13) or (14) above;
R₄, R₅, R₆ and R₇ are each independently as defined in paragraph (16) above.

In a particular group of compounds of formula Ia defined herein:
R₂ is as defined in paragraph (5) above;
R₃ is as defined in any one of paragraphs (10), (11), (12), (13) or (14) above;
R₄, R₅, R₆ and R₇ are each independently as defined in paragraph (17) above.

In a particular group of compounds of formula Ia defined herein:
R₂ is as defined in paragraph (5) above;
R₃ is as defined in any one of paragraphs (10), (11), (12), (13) or (14) above;
R₄, R₅, R₆ and R₇ are each independently as defined in paragraph (18) above.

In a particular group of compounds of formula Ia defined herein:
R₂ is as defined in paragraph (5) above;
R₃ is as defined in any one of paragraphs (10), (11), (12), (13) or (14) above;
R₄, R₅, R₆ and R₇ are each independently as defined in paragraph (19) above.

In a particular group of compounds of formula Ia defined herein:
R₂ is as defined in paragraph (5) above;
R₃ is as defined in any one of paragraphs (10), (11), (12), (13) or (14) above;
R₄, R₅, R₆ and R₇ are each independently as defined in paragraph (20) above.

In a particular group of compounds of formula Ia defined herein:
R₂ is as defined in paragraph (4) above;
R₃ is as defined in any one of paragraphs (21), (22), (23), (24), (25), (26), (27), (28), (29), (30), (31), (32), (33), (34) or (35) above;
R₄, R₅, R₆ and R₇ are each independently as defined in paragraph (15) above.

In a particular group of compounds of formula Ia defined herein:
R₂ is as defined in paragraph (4) above;
R₃ is as defined in any one of paragraphs (21), (22), (23), (24), (25), (26), (27), (28), (29), (30), (31), (32), (33), (34) or (35) above;
R₄, R₅, R₆ and R₇ are each independently as defined in paragraph (16) above.

In a particular group of compounds of formula Ia defined herein:
R₂ is as defined in paragraph (4) above;
R₃ is as defined in any one of paragraphs (21), (22), (23), (24), (25), (26), (27), (28), (29), (30), (31), (32), (33), (34) or (35) above;
R₄, R₅, R₆ and R₇ are each independently as defined in paragraph (17) above.

In a particular group of compounds of formula Ia defined herein:
R₂ is as defined in paragraph (4) above;
R₃ is as defined in any one of paragraphs (21), (22), (23), (24), (25), (26), (27), (28), (29), (30), (31), (32), (33), (34) or (35) above;
R₄, R₅, R₆ and R₇ are each independently as defined in paragraph (18) above.

In a particular group of compounds of formula Ia defined herein:
R₂ is as defined in paragraph (4) above;
R₃ is as defined in any one of paragraphs (21), (22), (23), (24), (25), (26), (27), (28), (29), (30), (31), (32), (33), (34) or (35) above;
R₄, R₅, R₆ and R₇ are each independently as defined in paragraph (19) above.

In a particular group of compounds of formula Ia defined herein:
R₂ is as defined in paragraph (4) above;
R₃ is as defined in any one of paragraphs (21), (22), (23), (24), (25), (26), (27), (28), (29), (30), (31), (32), (33), (34) or (35) above;
R₄, R₅, R₆ and R₇ are each independently as defined in paragraph (20) above.

In a particular group of compounds of formula Ia defined herein:
R₂ is as defined in paragraph (5) above;
R₃ is as defined in any one of paragraphs (21), (22), (23), (24), (25), (26), (27), (28), (29), (30), (31), (32), (33), (34) or (35) above;
R₄, R₅, R₆ and R₇ are each independently as defined in paragraph (15) above.

In a particular group of compounds of formula Ia defined herein:
R₂ is as defined in paragraph (5) above;
R₃ is as defined in any one of paragraphs (21), (22), (23), (24), (25), (26), (27), (28), (29), (30), (31), (32), (33), (34) or (35) above;
R₄, R₅, R₆ and R₇ are each independently as defined in paragraph (16) above.

In a particular group of compounds of formula Ia defined herein:
R₂ is as defined in paragraph (5) above;
R₃ is as defined in any one of paragraphs (21), (22), (23), (24), (25), (26), (27), (28), (29), (30), (31), (32), (33), (34) or (35) above;
R₄, R₅, R₆ and R₇ are each independently as defined in paragraph (17) above.

In a particular group of compounds of formula Ia defined herein:
R₂ is as defined in paragraph (5) above;
R₃ is as defined in any one of paragraphs (21), (22), (23), (24), (25), (26), (27), (28), (29), (30), (31), (32), (33), (34) or (35) above;
R₄, R₅, R₆ and R₇ are each independently as defined in paragraph (18) above.

In a particular group of compounds of formula Ia defined herein:
R₂ is as defined in paragraph (5) above;
R₃ is as defined in any one of paragraphs (21), (22), (23), (24), (25), (26), (27), (28), (29), (30), (31), (32), (33), (34) or (35) above;
R₄, R₅, R₆ and R₇ are each independently as defined in paragraph (19) above.

In a particular group of compounds of formula Ia defined herein:
R₂ is as defined in paragraph (5) above;
R₃ is as defined in any one of paragraphs (21), (22), (23), (24), (25), (26), (27), (28), (29), (30), (31), (32), (33), (34) or (35) above;
R₄, R₅, R₆ and R₇ are each independently as defined in paragraph (20) above.

In a particular group of compounds of formula Ia defined herein:
R₂ is as defined in paragraph (4) above;
R₃ is as defined in any one of paragraphs (7), (8), (9), (10), (11), (12), (13) or (14) above;
R₄, R₅, R₆ and R₇ are each independently as defined in paragraph (36) above.

In a particular group of compounds of formula Ia defined herein:
R₂ is as defined in paragraph (4) above;
R₃ is as defined in any one of paragraphs (10), (11), (12), (13) or (14) above;
R₄, R₅, R₆ and R₇ are each independently as defined in paragraph (36) above.

In a particular group of compounds of formula Ia defined herein:
R₂ is as defined in paragraph (4) above;
R₃ is as defined in any one of paragraphs (10), (11), (12), (13) or (14) above;
R₄, R₅, R₆ and R₇ are each independently as defined in paragraph (37) above.

In a particular group of compounds of formula Ia defined herein:
R₂ is as defined in paragraph (4) above;
R₃ is as defined in any one of paragraphs (10), (11), (12), (13) or (14) above;
R₄, R₅, R₆ and R₇ are each independently as defined in paragraph (38) above.

In a particular group of compounds of formula Ia defined herein:
R₂ is as defined in paragraph (4) above;
R₃ is as defined in any one of paragraphs (10), (11), (12), (13) or (14) above;
R₄, R₅, R₆ and R₇ are each independently as defined in paragraph (39) above.

In a particular group of compounds of formula Ia defined herein:
R₂ is as defined in paragraph (4) above;
R₃ is as defined in any one of paragraphs (10), (11), (12), (13) or (14) above;
R₄, R₅, R₆ and R₇ are each independently as defined in paragraph (40) above.

In a particular group of compounds of formula Ia defined herein:
R₂ is as defined in paragraph (5) above;
R₃ is as defined in any one of paragraphs (7), (8), (9), (10), (11), (12), (13) or (14) above;
R₄, R₅, R₆ and R₇ are each independently as defined in paragraph (36) above.

In a particular group of compounds of formula Ia defined herein:
R₂ is as defined in paragraph (5) above;
R₃ is as defined in any one of paragraphs (10), (11), (12), (13) or (14) above;
R₄, R₅, R₆ and R₇ are each independently as defined in paragraph (36) above.

In a particular group of compounds of formula Ia defined herein:
R₂ is as defined in paragraph (5) above;
R₃ is as defined in any one of paragraphs (10), (11), (12), (13) or (14) above;
R₄, R₅, R₆ and R₇ are each independently as defined in paragraph (37) above.

In a particular group of compounds of formula Ia defined herein:
R₂ is as defined in paragraph (5) above;
R₃ is as defined in any one of paragraphs (10), (11), (12), (13) or (14) above;
R₄, R₅, R₆ and R₇ are each independently as defined in paragraph (38) above.

In a particular group of compounds of formula Ia defined herein:
R₂ is as defined in paragraph (5) above;
R₃ is as defined in any one of paragraphs (10), (11), (12), (13) or (14) above;
R₄, R₅, R₆ and R₇ are each independently as defined in paragraph (39) above.

In a particular group of compounds of formula Ia defined herein:
R₂ is as defined in paragraph (5) above;
R₃ is as defined in any one of paragraphs (10), (11), (12), (13) or (14) above;
R₄, R₅, R₆ and R₇ are each independently as defined in paragraph (40) above.

In a particular group of compounds of formula Ia defined herein:
R₂ is as defined in paragraph (4) above;
R₃ is as defined in any one of paragraphs (21), (22), (23), (24), (25), (26), (27), (28), (29), (30), (31), (32), (33), (34) or (35) above;
R₄, R₅, R₆ and R₇ are each independently as defined in paragraph (36) above.

In a particular group of compounds of formula Ia defined herein:
R₂ is as defined in paragraph (4) above;
R₃ is as defined in any one of paragraphs (21), (22), (23), (24), (25), (26), (27), (28), (29), (30), (31), (32), (33), (34) or (35) above;
R₄, R₅, R₆ and R₁ are each independently as defined in paragraph (36) above.

In a particular group of compounds of formula Ia defined herein:
R₂ is as defined in paragraph (4) above;
R₃ is as defined in any one of paragraphs (21), (22), (23), (24), (25), (26), (27), (28), (29), (30), (31), (32), (33), (34) or (35) above;
R₄, R₅, R₆ and R₁ are each independently as defined in paragraph (37) above.

In a particular group of compounds of formula Ia defined herein:
R₂ is as defined in paragraph (4) above;
R₃ is as defined in any one of paragraphs (21), (22), (23), (24), (25), (26), (27), (28), (29), (30), (31), (32), (33), (34) or (35) above;
R₄, R₅, R₆ and R₇ are each independently as defined in paragraph (38) above.

In a particular group of compounds of formula Ia defined herein:
R₂ is as defined in paragraph (4) above;
R₃ is as defined in any one of paragraphs (21), (22), (23), (24), (25), (26), (27), (28), (29), (30), (31), (32), (33), (34) or (35) above;
R₄, R₅, R₆ and R₇ are each independently as defined in paragraph (39) above.

In a particular group of compounds of formula Ia defined herein:
R₂ is as defined in paragraph (4) above;
R₃ is as defined in any one of paragraphs (21), (22), (23), (24), (25), (26), (27), (28), (29), (30), (31), (32), (33), (34) or (35) above;
R₄, R₅, R₆ and R₇ are each independently as defined in paragraph (40) above.

In a particular group of compounds of formula Ia defined herein:
R₂ is as defined in paragraph (5) above;
R₃ is as defined in any one of paragraphs (21), (22), (23), (24), (25), (26), (27), (28), (29), (30), (31), (32), (33), (34) or (35) above;
R₄, R₅, R₆ and R₇ are each independently as defined in paragraph (36) above.

In a particular group of compounds of formula Ia defined herein:
R₂ is as defined in paragraph (5) above;
R₃ is as defined in any one of paragraphs (21), (22), (23), (24), (25), (26), (27), (28), (29), (30), (31), (32), (33), (34) or (35) above;
R₄, R₅, R₆ and R₇ are each independently as defined in paragraph (36) above.

In a particular group of compounds of formula Ia defined herein:
R₂ is as defined in paragraph (5) above;
R₃ is as defined in any one of paragraphs (21), (22), (23), (24), (25), (26), (27), (28), (29), (30), (31), (32), (33), (34) or (35) above;
R₄, R₅, R₆ and R₇ are each independently as defined in paragraph (37) above.

In a particular group of compounds of formula Ia defined herein:
R₂ is as defined in paragraph (5) above;
R₃ is as defined in any one of paragraphs (21), (22), (23), (24), (25), (26), (27), (28), (29), (30), (31), (32), (33), (34) or (35) above;
R₄, R₅, R₆ and R₇ are each independently as defined in paragraph (38) above.

In a particular group of compounds of formula Ia defined herein:
R₂ is as defined in paragraph (5) above;
R₃ is as defined in any one of paragraphs (21), (22), (23), (24), (25), (26), (27), (28), (29), (30), (31), (32), (33), (34) or (35) above;
R₄, Re, R₆ and R₇ are each independently as defined in paragraph (39) above.

In a particular group of compounds of formula Ia defined herein:
R₂ is as defined in paragraph (5) above;
R₃ is as defined in any one of paragraphs (21), (22), (23), (24), (25), (26), (27), (28), (29), (30), (31), (32), (33), (34) or (35) above;
R₄, R₅, R₆ and R₇ are each independently as defined in paragraph (40) above.

In a particular group of compounds of the invention, the compound is a compound of formula Ib shown below (a sub formula of Formula I), or a pharmaceutically acceptable salt thereof:

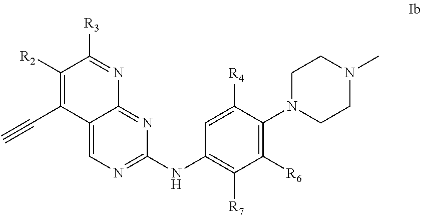

wherein R₂, R₃, R₄, R₅, R₆ and R₇ each have any one of the definitions set out herein.

Suitably, in any of the definitions of formula Ib set out herein, R₂ is as defined in formula I above or is as defined in any one of paragraphs (4), (5) or (6) above. In a particular group of compounds of formula Ib, R₂ is as defined in paragraph (4) above. In another particular group of compounds of formula Ib, R₂ is as defined in paragraph (5) above. In another particular group of compounds of formula Ib, R₂ is as defined in paragraph (6) above.

Suitably, in any of the definitions of formula Ib set out herein, R₃ is as defined in formula I above or is as defined in any one of paragraphs (7), (8), (9), (10), (11), (12), (13) or (14) above. In a particular group of compounds of formula Ib, R₃ is as defined in paragraph (7) above. In another particular group of compounds of formula Ib, R₃ is as defined in paragraph (8) above. In another particular group of compounds of formula Ib, R₃ is as defined in paragraph (9) above. In another particular group of compounds of formula Ib, R₃ is as defined in paragraph (10) above. In another particular group of compounds of formula Ib, R₃ is as defined in paragraph (11) above. In another particular group of compounds of formula Ib, R₃ is as defined in paragraph (12) above. In another particular group of compounds of formula Ib, R₃ is as defined in paragraph (13) above. In another particular group of compounds of formula Ib, R₃ is as defined in paragraph (14) above.

More suitably, in any of the definitions of formula Ib set out herein, R₃ is as defined in formula I above or is as defined in any one of paragraphs (21), (22), (23), (24), (25), (26), (27), (28), (29), (30), (31), (32), (33), (34) or (35) above. In a particular group of compounds of formula Ib, R₃ is as defined in paragraph (21) above. In another particular group of compounds of formula Ib, R₃ is as defined in paragraph (22) above. In another particular group of compounds of formula Ib, R₃ is as defined in paragraph (23) above. In a particular group of compounds of formula Ib, R₃ is as defined in paragraph (24) above. In another particular group of compounds of formula Ib, R₃ is as defined in paragraph (25) above. In another particular group of compounds of formula Ib, R₃ is as defined in paragraph (26) above. In another particular group of compounds of formula Ib, R₃ is as defined in paragraph (27) above.

In another particular group of compounds of formula Ib, R₃ is as defined in paragraph (28) above. In another particular group of compounds of formula Ib, R₃ is as defined in paragraph (29) above. In another particular group of compounds of formula Ib, R₃ is as defined in paragraph (30) above. In another particular group of compounds of of formula Ib, R₃ is as defined in paragraph (31) above. In another particular group of compounds of formula Ib, R₃ is as defined in paragraph (32) above. In another particular group of compounds of formula Ib, R₃ is as defined in paragraph (33) above. In another particular group of compounds of formula Ib, R₃ is as defined in paragraph (34) above. In another particular group of compounds of formula Ib, R₃ is as defined in paragraph (35) above.

Suitably, in any of the definitions of formula Ib set out herein, R₄ and R are as defined for formula I above or are as defined in any one of paragraphs (15), (16), (17), (18), (19) or (20) above. In a particular group of compounds of formula Ib, R₄ and R are as defined in paragraph (15) above. In another particular group of compounds of formula Ib, R₄ and R₆ are as defined in paragraph (16) above. In another particular group of compounds of formula Ib, R₄ and R are as defined in paragraph (17) above. In another particular group of compounds of formula Ib, R₄ and R₆ are as defined in paragraph (18) above. In another particular group of compounds of formula Ib, R₄ and R₆ are as defined in paragraph (19) above. In another particular group of compounds of formula Ib, R₄ and R₆ are as defined in paragraph (20) above.

In another particular group of compounds of formula Ib, one of R₄ and R₆ is hydrogen and the other is as defined in any one of paragraphs (15), (16), (17), (18), (19) or (20) above.

Suitably, in any of the definitions of formula Ib set out herein, R₇ is as defined for formula I above or are as defined in any one of paragraphs (15), (16), (17), (18), (19) or (20) above. In a particular group of compounds of formula Ib, R₇ is as defined in paragraph (15) above. In another particular group of compounds of formula Ib, R₇ is as defined in paragraph (16) above. In another particular group of compounds of formula Ib, R₇ is as defined in paragraph (17) above. In another particular group of compounds of formula Ib, R₇ is as defined in paragraph (18) above. In another particular group of compounds of formula Ib, R₇ is as defined in paragraph (19) above. In another particular group of compounds of formula Ib, R₇ is as defined in paragraph (20) above.

In a particular group of compounds of formula Ib defined herein:
R₂ is as defined in paragraph (4) above;
R₃ is as defined in any one of paragraphs (7), (8), (9), (10), (11), (12), (13) or (14) above;
R₄, R₆ and R₇ are each independently as defined in paragraph (15) above.

In a particular group of compounds of formula Ib defined herein:
R₂ is as defined in paragraph (4) above;
R₃ is as defined in any one of paragraphs (10), (11), (12), (13) or (14) above;
R₄, R₆ and R₇ are each independently as defined in paragraph (16) above.

In a particular group of compounds of formula Ib defined herein:
R₂ is as defined in paragraph (4) above;
R₃ is as defined in any one of paragraphs (10), (11), (12), (13) or (14) above;
R₄, R₆ and R₇ are each independently as defined in paragraph (17) above.

In a particular group of compounds of formula Ib defined herein:
R₂ is as defined in paragraph (4) above;
R₃ is as defined in any one of paragraphs (10), (11), (12), (13) or (14) above;
R₄, R₆ and R₇ are each independently as defined in paragraph (18) above.

In a particular group of compounds of formula Ib defined herein:
R₂ is as defined in paragraph (4) above;
R₃ is as defined in any one of paragraphs (10), (11), (12), (13) or (14) above;
R₄, R₆ and R₇ are each independently as defined in paragraph (19) above.

In a particular group of compounds of formula Ib defined herein:
R₂ is as defined in paragraph (4) above;
R₃ is as defined in any one of paragraphs (10), (11), (12), (13) or (14) above;
R₄, R₆ and R₇ are each independently as defined in paragraph (20) above.

In a particular group of compounds of formula Ib defined herein:
R₂ is as defined in paragraph (5) above;
R₃ is as defined in any one of paragraphs (7), (8), (9), (10), (11), (12), (13) or (14) above;
R₄, R₆ and R₇ are each independently as defined in paragraph (15) above.

In a particular group of compounds of formula Ib defined herein:
R₂ is as defined in paragraph (5) above;
R₃ is as defined in any one of paragraphs (10), (11), (12), (13) or (14) above;
R₄, R₅ and R₇ are each independently as defined in paragraph (16) above.

In a particular group of compounds of formula Ib defined herein:
R₂ is as defined in paragraph (5) above;
R₃ is as defined in any one of paragraphs (10), (11), (12), (13) or (14) above;
R₄, R₅ and R₇ are each independently as defined in paragraph (17) above.

In a particular group of compounds of formula Ib defined herein:
R₂ is as defined in paragraph (5) above;
R₃ is as defined in any one of paragraphs (10), (11), (12), (13) or (14) above;
R₄, R₅ and R₇ are each independently as defined in paragraph (18) above.

In a particular group of compounds of formula Ib defined herein:
R₂ is as defined in paragraph (5) above;
R₃ is as defined in any one of paragraphs (10), (11), (12), (13) or (14) above;
R₄, R₅ and R₇ are each independently as defined in paragraph (19) above.

In a particular group of compounds of formula Ib defined herein:
R₂ is as defined in paragraph (5) above;
R₃ is as defined in any one of paragraphs (10), (11), (12), (13) or (14) above;
R₄, R₆ and R₇ are each independently as defined in paragraph (20) above.

In a particular group of compounds of formula Ib defined herein:
R₂ is as defined in paragraph (5) above;
R₃ is as defined in any one of paragraphs (10), (11), (12), (13) or (14) above;
R₄, R₆ and R₇ are each hydrogen.

In a particular group of compounds of formula Ib defined herein:
R₂ is as defined in paragraph (4) above;
R₃ is as defined in any one of paragraphs (21), (22), (23), (24), (25), (26), (27), (28), (29), (30), (31), (32), (33), (34) or (35) above;
R₄, R₆ and R₇ are each independently as defined in paragraph (15) above.

In a particular group of compounds of formula Ib defined herein:
R₂ is as defined in paragraph (4) above;
R₃ is as defined in any one of paragraphs (21), (22), (23), (24), (25), (26), (27), (28), (29), (30), (31), (32), (33), (34) or (35) above;
R₄, R₆ and R₇ are each independently as defined in paragraph (16) above.

In a particular group of compounds of formula Ib defined herein:
R₂ is as defined in paragraph (4) above;
R₃ is as defined in any one of paragraphs (21), (22), (23), (24), (25), (26), (27), (28), (29), (30), (31), (32), (33), (34) or (35) above;
R₄, R₅ and R₇ are each independently as defined in paragraph (17) above.

In a particular group of compounds of formula Ib defined herein:
R₂ is as defined in paragraph (4) above;
R₃ is as defined in any one of paragraphs (21), (22), (23), (24), (25), (26), (27), (28), (29), (30), (31), (32), (33), (34) or (35) above;
R₄, R₅ and R₇ are each independently as defined in paragraph (18) above.

In a particular group of compounds of formula Ib defined herein:
R₂ is as defined in paragraph (4) above;
R₃ is as defined in any one of paragraphs (21), (22), (23), (24), (25), (26), (27), (28), (29), (30), (31), (32), (33), (34) or (35) above;
R₄, R₆ and R₇ are each independently as defined in paragraph (19) above.

In a particular group of compounds of formula Ib defined herein:
R₂ is as defined in paragraph (4) above;
R₃ is as defined in any one of paragraphs (21), (22), (23), (24), (25), (26), (27), (28), (29), (30), (31), (32), (33), (34) or (35) above;
R₄, R₆ and R₇ are each independently as defined in paragraph (20) above.

In a particular group of compounds of formula Ib defined herein:
R₂ is as defined in paragraph (5) above;
R₃ is as defined in any one of paragraphs (21), (22), (23), (24), (25), (26), (27), (28), (29), (30), (31), (32), (33), (34) or (35) above;
R₄, R₆ and R₇ are each independently as defined in paragraph (15) above.

In a particular group of compounds of formula Ib defined herein:
R₂ is as defined in paragraph (5) above;
R₃ is as defined in any one of paragraphs (21), (22), (23), (24), (25), (26), (27), (28), (29), (30), (31), (32), (33), (34) or (35) above;
R₄, R₆ and R₇ are each independently as defined in paragraph (16) above.

In a particular group of compounds of formula Ib defined herein:
R₂ is as defined in paragraph (5) above;
R₃ is as defined in any one of paragraphs (21), (22), (23), (24), (25), (26), (27), (28), (29), (30), (31), (32), (33), (34) or (35) above;
R₄, R₆ and R₇ are each independently as defined in paragraph (17) above.

In a particular group of compounds of formula Ib defined herein:
R₂ is as defined in paragraph (5) above;
R₃ is as defined in any one of paragraphs (21), (22), (23), (24), (25), (26), (27), (28), (29), (30), (31), (32), (33), (34) or (35) above;
R₄, R₅ and R₇ are each independently as defined in paragraph (18) above.

In a particular group of compounds of formula Ib defined herein:
R₂ is as defined in paragraph (5) above;
R₃ is as defined in any one of paragraphs (21), (22), (23), (24), (25), (26), (27), (28), (29), (30), (31), (32), (33), (34) or (35) above;
R₄, R₆ and R₇ are each independently as defined in paragraph (19) above.

In a particular group of compounds of formula Ib defined herein:
R₂ is as defined in paragraph (5) above;
R₃ is as defined in any one of paragraphs (21), (22), (23), (24), (25), (26), (27), (28), (29), (30), (31), (32), (33), (34) or (35) above;
R₄, R₆ and R₇ are each independently as defined in paragraph (20) above.

In a particular group of compounds of formula Ib defined herein:
R₂ is as defined in paragraph (5) above;
R₃ is as defined in any one of paragraphs (21), (22), (23), (24), (25), (26), (27), (28), (29), (30), (31), (32), (33), (34) or (35) above;
R₄, R₆ and R₇ are each hydrogen.

Particular compounds of the present invention include any of the compounds described in the example section of the present application, or a pharmaceutically acceptable salt, hydrate or solvate thereof, and, in particular, any of the following:
5-ethynyl-N-(2-methoxyphenyl)pyrido[2,3-d]pyrimidin-2-amine;
N¹-(2-(dimethylamino)ethyl)-N⁴-(5-ethynyl-7-methoxypyrido[2,3-d]pyrimidin-2-yl)-N¹-methylbenzene-1,4-diamine;
5-ethynyl-7-methoxy-N-(4-(4-methylpiperazin-1-yl)phenyl)pyrido[2,3-d]pyrimidin-2-amine;
N-(3-{[cyclobutyl(methyl)amino]methyl}phenyl)-5-ethynyl-7-methoxypyrido[2,3-d]pyrimidin-2-amine;
N³-{5-ethynyl-7-methoxypyrido[2,3-d]pyrimidin-2-yl}-N¹, N-dimethyl-6-(4-methylpiperazin-1-yl)benzene-1,3-diamine;
N1-{5-ethynyl-7-methoxypyrido[2,3-d]pyrimidin-2-yl}-4-(4-methylpiperazin -1-yl)-N³-phenylbenzene-1,3-diamine;
N-{[3-({5-ethynyl-7-methoxypyrido[2,3-d]pyrimidin-2-yl}amino)phenyl]methyl}-N-methylacetamide;
N³-cyclobutyl-N¹-{5-ethynyl-7-methoxypyrido[2,3-d]pyrimidin-2-yl}-4-(4-methylpiperazin-1-yl)benzene-1,3-diamine;
N-{3-[(dimethylamino)methyl]phenyl}-5-ethynyl-7-methoxypyrido[2,3-d]pyrimidin-2-amine;
N³-(cyclobutylmethyl)-N¹-{5-ethynyl-7-methoxypyrido[2,3-d]pyrimidin-2-yl}-4-(4-methylpiperazin-1-yl)benzene-1,3-diamine;
N¹-{5-ethynyl-7-methoxypyrido[2,3-d]pyrimidin-2-yl}-4-(4-methylpiperazin-1-yl)-N³-[(1-methylpyrazol-3-yl)methyl]benzene-1,3-diamine;
5-ethynyl-N-[4-(4-methylpiperazin-1-yl)phenyl]-7-phenoxypyrido[2,3-d]pyrimidin-2-amine;
5-ethynyl-N-[4-(4-methylpiperazin-1-yl)phenyl]-7-(1,3-thiazol-4-ylmethoxy)pyrido[2,3-d]pyrimidin-2-amine;
7-(cyclohexyloxy)-5-ethynyl-N-(4-(4-methylpiperazin-1-yl)phenyl)pyrido[2,3-d]pyrimidin-2-amine;
5-ethynyl-N-(4-(4-methylpiperazin-1-yl) phenyl)-7-((tetrahydro-2H-pyran-4-yl)oxy) pyrido[2, 3-d]pyrimidin-2-amine;
7-cyclopropoxy-5-ethynyl-N-[4-(4-methylpiperazin-1-yl) phenyl]pyrido[2,3-d]pyrimidin-2-amine;
5-ethynyl-7-isopropoxy-N-[4-(4-methylpiperazin-1-yl)phenyl]pyrido[2,3-d]pyrimidin-2-amine;
7-cyclobutoxy-5-ethynyl-N-[4-(4-methylpiperazin-1-yl)phenyl]pyrido[2,3-d]pyrimidin-2-amine;
7-cyclopropoxy-5-ethynyl-N-[4-(4-methylpiperazin-1-yl) phenyl]pyrido[2,3-d]pyrimidin-2-amine;
7-(benzyloxy)-5-ethynyl-N-[4-(4-methylpiperazin-1-yl)phenyl]pyrido[2,3-d]pyrimidin-2-amine;
5-ethynyl-N-[4-(4-methylpiperazin-1-yl)phenyl]-7-(2-phenylethoxy)pyrido[2,3-d]pyrimidin-2-amine;
5-ethynyl-N-[4-(4-methylpiperazin-1-yl)phenyl]-7-(3-phenylpropoxy)pyrido[2,3-d]pyrimidin-2-amine
7-(cyclopentylmethoxy)-5-ethynyl-6-methyl-N-[4-(4-methylpiperazin-1-yl) phenyl]pyrido[2,3-d]pyrimidin-2-amine;
N²-(4-{[2-(dimethylamino)ethyl](methyl)amino}phenyl)-5-ethynyl-N⁷,N⁷-dimethylpyrido[2,3-d]pyrimidine-2,7-diamine;
N²-(4-{[2-(dimethylamino)ethyl](methyl)amino}phenyl)-5-ethynyl-N⁷-phenylpyrido[2,3-d]pyrimidine-2,7-diamine;
5-ethynyl-N⁷-(1-methylmidazol-2-yl)-N²-[4-(4-methylpiperazin-1-yl)phenyl]pyrido[2,3-d]pyrimidine-2,7-diamine;
N⁷-(1-cyclopentylmidazol-2-yl)-5-ethynyl-N²-[4-(4-methylpiperazin-1-yl)phenyl]pyrido[2,3-d]pyrimidine-2,7-diamine;
N-{2-[(4-{[2-(dimethylamino)ethyl](methyl)amino}phenyl)amino]-5-ethynyl)pyrido[2,3-d]pyrimidin-7-yl}acetamide;
N-(5-ethynyl-2-{[4-(4-methylpiperazin-1-yl)phenyl]amino}pyrido[2,3-d]pyrimidin-7-yl)benzamide;
N-(5-ethynyl-2-{[4-(4-methylpiperazin-1-yl)phenyl]amino}pyrido[2,3-d]pyrimidin-7-yl)-2-phenylacetamide;
1-(5-ethynyl-2-{[4-(4-methylpiperazin-1-yl)phenyl]amino}pyrido[2,3-d]pyrimidin-7-yl)pyrrolidin-2-one:
N-(5-ethynyl-2-{[4-(4-methylpiperazin-1-yl)phenyl]amino}pyrido[2,3-d]pyrimidin-7-yl)-3-phenylpropanamide;
N-{2-[(4-{[2-(dimethylamino)ethyl](methyl)amino}phenyl)amino]-5-ethynyl)pyrido[2,3-d]pyrimidin-7-yl}methanesulfonamide;
N¹-[2-(dimethylamino)ethyl]-N⁴-[5-ethynyl-7-(4-methylpyrazol-1-yl)pyrido[2,3-d]pyrimidin-2-yl]-N¹-methylbenzene-1,4-diamine;
5-ethynyl-7-(imidazol-1-yl)-N-[4-(4-methylpiperazin-1-yl)phenyl]pyrido[2,3-d]pyrimidin-2-amine;
5-ethynyl-N-[4-(4-methylpiperazin-1-yl)phenyl]-7-(pyrrol-1-yl)pyrido[2,3-d]pyrimidin-2-amine;
1-(5-ethynyl-2-{[4-(4-methylpiperazin-1-yl)phenyl]amino}pyrido[2,3-d]pyrimidin-7-yl)-3,3-dimethylurea;
1-cyclopentyl-3-(5-ethynyl-2-((4-(4-methylpiperazin-1-yl)phenyl)amino)pyrido[2,3-d]pyrimidin-7-yl)urea;
1-benzyl-3-(5-ethynyl-2-{[4-(4-methylpiperazin-1-yl)phenyl]amino}pyrido[2,3-d]pyrimidin-7-yl)urea;
3-(cyclopentylmethyl)-1-(5-ethynyl-2-{[4-(4-methylpiperazin-1-yl)phenyl]amino}pyrido[2,3-d]pyrimidin-7-yl)urea;
3-cyclobutyl-1-(5-ethynyl-2-{[4-(4-methylpiperazin-1-yl)phenyl]amino}pyrido[2,3-d]pyrimidin-7-yl)urea;
1-(5-ethynyl-2-{[4-(4-methylpiperazin-1-yl)phenyl]amino}pyrido[2,3-d]pyrimidin-7-yl)-3-(oxolan-3-yl)urea;
1-(5-ethynyl-2-{[4-(4-methylpiperazin-1-yl)phenyl]amino}pyrido[2,3-d]pyrimidin-7-yl)-3-(oxetan-3-yl)urea;
3-cyclopropyl-1-(5-ethynyl-2-{[4-(4-methylpiperazin-1-yl)phenyl]amino}pyrido[2,3-d]pyrimidin-7-yl)urea;
1-(5-ethynyl-2-{[4-(4-methylpiperazin-1-yl)phenyl]amino}pyrido[2,3-d]pyrimidin-7-yl)-3-(1,3-oxazol-5-ylmethyl)urea;
1-(5-ethynyl-2-{[4-(4-methylpiperazin-1-yl)phenyl]amino}pyrido[2,3-d]pyrimidin-7-yl)-3-[(3-methylmidazol-4-yl)methyl]urea;
1-(5-ethynyl-2-{[4-(4-methylpiperazin-1-yl)phenyl]amino}pyrido[2,3-d]pyrimidin-7-yl)-3-(1,2-oxazol-5-ylmethyl)urea;
1-(5-ethynyl-2-{[4-(4-methylpiperazin-1-yl)phenyl]amino}pyrido[2,3-d]pyrimidin-7-yl)-3-[(2-methylpyrazol-3-yl)methyl]urea;
3-(5-ethynyl-2-{[4-(4-methylpiperazin-1-yl)phenyl]amino}pyrido[2,3-d]pyrimidin-7-yl)-1-(pyridin-2-ylmethyl)urea;
1-(5-ethynyl-2-{[4-(4-methylpiperazin-1-yl)phenyl]amino}pyrido[2,3-d]pyrimidin-7-yl)-3-(oxolan-2-ylmethyl)urea;
1-(5-ethynyl-2-{[4-(4-methylpiperazin-1-yl)phenyl]amino}pyrido[2,3-d]pyrimidin-7-yl)-3-(oxolan-3-ylmethyl)urea;
3-[(3R)-1-acetylpyrrolidin-3-yl]-1-(5-ethynyl-2-{[4-(4-methylpiperazin-1-yl)phenyl]amino}pyrido[2,3-d]pyrimidin-7-yl)urea;
3-[(3S)-1-acetylpyrrolidin-3-yl]-1-(5-ethynyl-2-{[4-(4-methylpiperazin-1-yl)phenyl]amino}pyrido[2,3-d]pyrimidin-7-yl)urea;
3-[(3R)-1-acetylpyrrolidin-3-yl]-1-(5-ethynyl-2-{[4-(4-methylpiperazin-1-yl)phenyl]amino}pyrido[2,3-d]pyrimidin-7-yl)urea;
1-(5-ethynyl-2-{[4-(4-methylpiperazin-1-yl)phenyl]amino}pyrido[2,3-d]pyrimidin-7-yl)-3-(oxetan-3-ylmethyl)urea;
N-(5-ethynyl-2-{[4-(4-methylpiperazin-1-yl)phenyl]amino}pyrido[2,3-d]pyrimidin-7-yl)pyrrolidine-1-carboxamide;
1-(5-ethynyl-2-{[4-(4-methylpiperazin-1-yl)phenyl]amino}pyrido[2,3-d]pyrimidin-7-yl)-3-(1-methanesulfonylazetidin-3-yl)urea;
N-(5-ethynyl-2-{[4-(4-methylpiperazin-1-yl)phenyl]amino}pyrido[2,3-d]pyrimidin-7-yl)-1,3-dihydroisoindole-2-carboxamide;
1-(5-ethynyl-2-{[4-(4-methylpiperazin-1-yl)phenyl]amino}pyrido[2,3-d]pyrimidin-7-yl)-3-(1H-imidazol-2-yl)urea;
1-(5-ethynyl-2-((4-(4-methylpiperazin-1-yl)phenyl)amino)pyrido[2,3-d]pyrimidin-7-yl)-3-(1-(trifluoromethyl)cyclopentyl)urea;
1-(5-ethynyl-2-((4-(4-methylpiperazin-1-yl)phenyl)amino)pyrido[2,3-d]pyrimidin-7-yl)-3-(3-methyloxetan-3-yl)urea;
1-(5-ethynyl-2-((4-(4-methylpiperazin-1-yl)phenyl)amino)pyrido[2,3-d]pyrimidin-7-yl)-3-(3-methyloxetan-3-yl)urea;
1-(2-((3-((cyclobutylmethyl)amino)-4-(4-methylpiperazin-1-yl)phenyl)amino)-5-ethynyl)pyrido[2,3-d]pyrimidin-7-yl)-3-cyclopentylurea;
1-(5-ethynyl-2-{[4-(4-methylpiperazin-1-yl)phenyl]amino}pyrido[2,3-d]pyrimidin-7-yl)-5-isopropylmidazolidin-2-one;
cyclopentyl (5-ethynyl-2-((4-(4-methylpiperazin-1-yl)phenyl)amino)pyrido[2,3-d]pyrimidin-7-yl)carbamate;
cyclohexyl (5-ethynyl-2-((4-(4-methylpiperazin-1-yl)phenyl)amino)pyrido[2,3-d]pyrimidin-7-yl)carbamate;
benzyl (5-ethynyl-2-((4-(4-methylpiperazin-1-yl)phenyl)amino)pyrido[2,3-d]pyrimidin-7-yl)carbamate;
3-(5-ethynyl-2-{[4-(4-methylpiperazin-1-yl)phenyl]amino}pyrido[2,3-d]pyrimidin-7-yl)-4-isopropyl-1,3-oxazolidin-2-one;
(4R)-3-(5-ethynyl-2-{[4-(4-methylpiperazin-1-yl)phenyl]amino}pyrido[2,3-d]pyrimidin-7-yl)-4-isopropyl-1,3-oxazolidin-2-one;
(4S)-3-(5-ethynyl-2-{[4-(4-methylpiperazin-1-yl) phenyl]amino}pyrido[2, 3-d]pyrimidin-7-yl)-4-isopropyl-1,3-oxazolidin-2-one;
3-(5-ethynyl-2-{[4-(4-methylpiperazin-1-yl)phenyl]amino}pyrido[2,3-d]pyrimidin-7-yl)-1,3-oxazolidin-2-one;
3-Cyclopentyl-1-(5-ethynyl-6-methyl-2-{[4-(4-methylpiperazin-1-yl)phenyl]amino}pyrido[2,3-d]pyrimidin-7-yl)urea;
3-Cyclopentyl-1-(5-ethynyl-2-{[4-(4-methylpiperazin-1-yl)phenyl]amino}pyrido[2, 3-d]pyrimidin-7-yl)-1-methylurea;
3-Cyclopentyl-1-(5-ethynyl-2-{[4-(4-methylpiperazin-1-yl)phenyl]amino}pyrido[2, 3-d]pyrimidin-7-yl)-3-methylurea;
3-Cyclopentyl-1-(5-ethynyl-2-([4-(morpholin-4-yl)phenyl]aminopyrido[2,3-d]pyrimidin-7-yl)urea;
3-Cyclopentyl-1-[5-ethynyl-2-({4-[4-(oxetan-3-yl)piperazin-1-yl]phenyl}amino)pyrido[2,3-d]pyrimidin-7-yl]urea;
N-[4-((7-[(Cyclopentylcarbamoyl)amino]-5-ethynyl)pyrido[2,3-d]pyrimidin-2-ylamino)phenyl]-N-methylacetamide;
2-(Azetidin-1-yl)-N-[4-((7-[(cyclopentylcarbamoyl)amino]-5-ethynyl)pyrido[2,3-d]pyrimidin-2-ylamino)phenyl]-N-methylacetamide;
3-Cyclopentyl-1-[2-((4-[2-(dimethylamino)ethoxy]phenylamino)-5-ethynyl)pyrido[2,3-d]pyrimidin-7-yl]urea;
3-Cyclopentyl-1-[5-ethynyl-2-((4-[(1-methylazetidin-3-yl)oxy]phenylamino)pyrido[2,3-d]pyrimidin-7-yl]urea;
3-Cyclopentyl-1-{5-ethynyl-2-[(4-methanesulfonylphenyl)amino]pyrido[2,3-d]pyrimidin-7-yl}urea;
3-Cyclopentyl-1-(2-([4-(dimethylsulfamoyl)phenyl]amino-5-ethynyl)pyrido[2,3-d]pyrimidin-7-yl)urea;
N-[4-({7-[(Cyclopentylcarbamoyl)amino]-5-ethynyl)pyrido[2,3-d]pyrimidin-2-yl}amino)phenyl]-2-(dimethylamino)-N-methylacetamide;
3-Cyclopentyl-1-(2-[(4-([2-(dimethylamino)ethyl](methyl)sulfamoyl)phenyl)amino]-5-ethynyl)pyrido[2,3-d]pyrimidin-7-ylurea;
3-Cyclopentyl-1-(5-ethynyl-2-[(3-([3-(methanesulfonylmethyl)azetidin-1-yl]methyl)phenyl)amino]pyrido[2,3-d]pyrimidin-7-ylurea;
3-Cyclopentyl-1-(5-ethynyl-2-[(3-([3-(methanesulfonylmethyl)pyrrolidin-1-yl]methyl)phenyl)amino]pyrido[2,3-d]pyrimidin-7-ylurea;
3-Cyclopentyl-1-(5-ethynyl-2-[(3-([4-(methanesulfonylmethyl)piperidin-1-yl]methyl)phenyl)amino]pyrido[2,3-d]pyrimidin-7-ylurea;
3-Cyclopentyl-1-{5-ethynyl-2-[(2-methyl-3,4-dihydro-1H-isoquinolin-5-yl)amino]pyrido[2,3-d]pyrimidin-7-yl}urea;
3-Cyclopentyl-1-[5-ethynyl-2-((2-[3-(methanesulfonylmethyl)pyrrolidin-1-yl]phenylamino)pyrido[2,3-d]pyrimidin-7-yl]urea;
3-Cyclopentyl-1-[5-ethynyl-2-({2-[4-(methanesulfonylmethyl)piperidin-1-yl]phenyl}amino)pyrido[2,3-d]pyrimidin-7-yl]urea;
1-(5-Ethynyl-2-{[4-(morpholin-4-yl)phenyl]amino}pyrido[2,3-d]pyrimidin-7-yl)-1,3-diazaspiro[4.4]nonan-2-one;
1-[5-Ethynyl-2-({4-[4-(oxetan-3-yl)piperazin-1-yl]phenyl}amino)pyrido[2,3-d]pyrimidin-7-yl]-1,3-diazaspiro[4.4]nonan-2-one;
N-{4-[(5-Ethynyl-7-{2-oxo-1,3-diazaspiro[4.4]nonan-1-yl}pyrido[2,3-d]pyrimidin-2-yl)amino]phenyl}-N-methylacetamide;
2-(Dimethylamino)-N-{4-[(5-ethynyl-7-{2-oxo-1,3-diazaspiro[4.4]nonan-1-yl}pyrido[2,3-d]pyrimidin-2-yl)amino]phenyl}-N-methylacetamide;
1-[2-({4-[2-(Dimethylamino)ethoxy]phenyl}amino)-5-ethynyl)pyrido[2,3-d]pyrimidin-7-yl]-1,3-diazaspiro[4.4]nonan-2-one;
1-{5-Ethynyl-2-[(4-methanesulfonylphenyl)amino]pyrido[2,3-d]pyrimidin-7-yl}-1,3-diazaspiro[4.4]nonan-2-one;
4-[(5-Ethynyl-7-{2-oxo-1,3-diazaspiro[4.4]nonan-1-yl}pyrido[2,3-d]pyrimidin-2-yl)amino]-N, N-dimethylbenzenesulfonamide;
N-[2-(dimethylamino)ethyl]-4-[(5-ethynyl-7-{2-oxo-1,3-diazaspiro[4.4]nonan-1-yl}pyrido[2,3-d]pyrimidin-2-yl)amino]-N-methylbenzenesulfonamide;
(5S)-1-(5-Ethynyl-2-{[4-(4-methylpiperazin-1-yl)phenyl]amino}pyrido[2,3-d]pyrimidin-7-yl)-5-isopropylmidazolidin-2-one;
(5R)-1-(5-Ethynyl-2-{[4-(4-methylpiperazin-1-yl)phenyl]amino}pyrido[2,3-d]pyrimidin-7-yl)-5-isopropylmidazolidin-2-one;
(5S)-1-(5-Ethynyl-2-{[4-(4-methylpiperazin-1-yl)phenyl]amino}pyrido[2,3-d]pyrimidin-7-yl)-5-(methoxymethyl)imidazolidin-2-one;
3-(5-Ethynyl-2-{[4-(4-methylpiperazin-1-yl)phenyl]amino}pyrido[2,3-d]pyrimidin-7-yl)-4-isopropyl-1-methylmidazolidin-2-one;
1-(5-Ethynyl-2-{[4-(morpholin-4-yl)phenyl]amino}pyrido[2,3-d]pyrimidin-7-yl)-3-methyl-1,3-diazaspiro[4.4]nonan-2-one;
1-[5-Ethynyl-2-({4-[4-(oxetan-3-yl)piperazin-1-yl]phenyl}amino)pyrido[2,3-d]pyrimidin-7-yl]-3-methyl-1,3-diazaspiro[4.4]nonan-2-one;
N-{4-[(5-Ethynyl-7-{3-methyl-2-oxo-1,3-diazaspiro[4.4]nonan-1-yl}pyrido[2,3-d]pyrimidin-2-yl)amino]phenyl}-N-methylacetamide;
2-(Dimethylamino)-N-{4-[(5-ethynyl-7-{3-methyl-2-oxo-1,3-diazaspiro[4.4]nonan-1-yl}pyrido[2,3-d]pyrimidin-2-yl)amino]phenyl}-N-methylacetamide;
1-[2-({4-[2-(Dimethylamino) ethoxy]phenyl}amino)-5-ethynyl)pyrido[2,3-d]pyrimidin-7-yl]-3-methyl-1,3-diazaspiro[4.4]nonan-2-one;
1-{5-Ethynyl-2-[(4-methanesulfonylphenyl)amino]pyrido [2,3-d]pyrimidin-7-yl}-3-methyl-1,3-diazaspiro [4.4]nonan-2-one;
4-[(5-Ethynyl-7-{3-methyl-2-oxo-1,3-diazaspiro[4.4]nonan-1-yl}pyrido[2,3-d]pyrimidin-2-yl)amino]-N, N-dimethylbenzenesulfonamide;
3-Cyclopentyl-1-[5-ethynyl-2-((2-[3-(methanesulfonylmethyl)azetidin-1-yl]phenylamino)pyrido[2,3-d]pyrimidin-7-yl]urea;
4-Cyclopentyl-3-(5-ethynyl-2-{[4-(4-methylpiperazin-1-yl]phenylamino}pyrido[2,3-d]pyrimidin-7-yl)-1,3-oxazolidin-2-one;
4-(Cyclopentylmethyl)-3-(5-ethynyl-2-{[4-(4-methylpiperazin-1-yl)phenyl]amino}pyrido[2,3-d]pyrimidin-7-yl)-1,3-oxazolidin-2-one;
3-(5-Ethynyl-2-{[4-(4-methylpiperazin-1-yl)phenyl]amino}pyrido[2,3-d]pyrimidin-7-yl)-5-isopropyl-1,3-oxazolidin-2-one;
1-(5-Ethynyl-2-{[4-(4-methylpiperazin-1-yl)phenyl]amino}pyrido[2,3-d]pyrimidin-7-yl)-3-oxa-1-azaspiro[4.4]nonan-2-one;
4-Cyclopentyl-3-(5-ethynyl-2-{[4-(piperazin-1-yl)phenyl]amino}pyrido[2,3-d]pyrimidin-7-yl)-1,3-oxazolidin-2-one;
3-(5-Ethynyl-2-{[4-(4-methylpiperazin-1-yl)phenyl]amino}pyrido[2,3-d]pyrimidin-7-yl)-4-(methoxymethyl)-1,3-oxazolidin-2-one;
1-cyclopentyl-3-(5-ethynyl-2-((2-methylsoindolin-4-yl)amino)pyrido[2,3-d]pyrimidin-7-yl)urea;
1-cyclopentyl-3-(5-ethynyl-2-((2-(3-((methylsulfonyl)methyl)piperidin-1-yl)phenyl)amino)pyrido[2,3-d]pyrimidin-7-yl)urea;
1-cyclopentyl-3-(5-ethynyl-2-((3-(methyl((1s,3s)-3-((methylsulfonyl)methyl)cyclobutyl)amino)phenyl)amino)pyrido[2,3-d]pyrimidin-7-yl)urea;
1-cyclopentyl-3-(5-ethynyl-2-((3-(methyl((1s,4s)-4-((methylsulfonyl)methyl)cyclohexyl)amino)phenyl)amino)pyrido[2,3-d]pyrimidin-7-yl)urea;
1-cyclopentyl-3-(5-ethynyl-2-((3-(methyl((1r,4r)-4-((methylsulfonyl)methyl)cyclohexyl)amino)phenyl)amino)pyrido[2,3-d]pyrimidin-7-yl)urea;
1-cyclopentyl-3-(5-ethynyl-2-((3-(methyl((1r,3r)-3-((methylsulfonyl)methyl)cyclobutyl)amino)phenyl)amino)pyrido[2,3-d]pyrimidin-7-yl)urea;
1-cyclopentyl-3-(5-ethynyl-2-((4-(4-methylpiperazin-1-yl)phenyl)amino)pyrido[2,3-d]pyrimidin-7-yl)-1,3-dimethylurea; and
5-cyclopentyl-1-(5-ethynyl-2-((4-(4-methylpiperazin-1-yl)phenyl)amino)pyrido[2,3-d]pyrimidin-7-yl)imidazolidin-2-one.

Though the present invention may relate to any compound or particular group of compounds defined herein by way of optional, preferred or suitable features or otherwise in terms of particular embodiments, the present invention may also relate to any compound or particular group of compounds that specifically excludes said optional, preferred or suitable features or particular embodiments.

Suitably, the present invention excludes any individual compounds not possessing the biological activity defined herein.

### Salts and Solvates

The compounds (including final products and intermediates) described herein may be isolated and used per se or may be isolated in the form of a salt, suitably pharmaceutically acceptable salts. It should be understood that the terms “salt(s)” and “salt form(s)” used by themselves or in conjunction with another term or terms encompasses all inorganic and organic salts, including industrially acceptable salts, as defined herein, and pharmaceutically acceptable salts, as defined herein, unless otherwise specified. As used herein, industrially acceptable salts are salts that are generally suitable for manufacturing and/or processing (including purification) as well as for shipping and storage, but may not be salts that are typically administered for clinical or therapeutic use. Industrially acceptable salts may be prepared on a laboratory scale, i.e. multi-gram or smaller, or on a larger scale, i.e. up to and including a kilogram or more.

Pharmaceutically acceptable salts, as used herein, are salts that are generally chemically and/or physically compatible with the other ingredients comprising a formulation, and/or are generally physiologically compatible with the recipient thereof. Pharmaceutically acceptable salts may be prepared on a laboratory scale, i.e. multi-gram or smaller, or on a larger scale, i.e. up to and including a kilogram or more. It should be understood that pharmaceutically acceptable salts are not limited to salts that are typically administered or approved by the FDA or equivalent foreign regulatory body for clinical or therapeutic use in humans. A practitioner of ordinary skill will readily appreciate that some salts are both industrially acceptable as well as pharmaceutically acceptable salts. It should be understood that all such salts, including mixed salt forms, are within the scope of the application.

In one embodiment, the compounds of Formula I and sub-formulae thereof are isolated as pharmaceutically acceptable salts.

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulfuric, phosphoric, trifluoroacetic, formic, citric or maleic acid. In addition a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

In general, salts of the present application can be prepared in situ during the isolation and/or purification of a compound (including intermediates), or by separately reacting the compound (or intermediate) with a suitable organic or inorganic acid or base (as appropriate) and isolating the salt thus formed. The degree of ionisation in the salt may vary from completely ionised to almost non-ionised. In practice, the various salts may be precipitated (with or without the addition of one or more co-solvents and/or anti-solvents) and collected by filtration or the salts may be recovered by evaporation of solvent(s). Salts of the present application may also be formed via a “salt switch” or ion exchange/double displacement reaction, i.e. reaction in which one ion is replaced (wholly or in part) with another ion having the same charge. One skilled in the art will appreciate that the salts may be prepared and/or isolated using a single method or a combination of methods.

Representative salts include, but are not limited to, acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate, trifluoroacetate and the like. Other examples of representative salts include alkali or alkaline earth metal cations such as, but not limited to, sodium, lithium, potassium, calcium, magnesium, and the like, as well as non-toxic ammonium, quaternary ammonium and amine cations including, but not limited to, ammonium, tetramethylammonium, tetraethylammonium, lysine, arginine, benzathine, choline, tromethamine, diolamine, glycine, meglumine, olamine and the like.

Certain compounds of the Formula I and sub-formulae thereof may exist in solvated as well as unsolvated forms such as, for example, hydrated forms. It is to be understood that the invention encompasses all such solvated forms that possess the biological activity described herein.

### Polymorphs

It is also to be understood that certain compounds of the Formula I and sub-formulae thereof may exhibit polymorphism, and that the invention encompasses all such forms that possess the biological activity described herein.

### N-Oxides

Compounds of the Formula I and sub-formulae thereof containing an amine function may also form N-oxides. A reference herein to a compound of the Formula I and sub-formulae thereof that contains an amine function also includes the N-oxide. Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle. N-Oxides can be formed by treatment of the corresponding amine with an oxidizing agent such as, but not limited to, hydrogen peroxide or a per-acid (e.g. a peroxycarboxylic acid), see for example Advanced Organic Chemistry, by Jerry March, 4^{th} Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (mCPBA), for example, in an inert solvent such as, but not limited to, dichloromethane.

### Tautomers

Compounds of the Formula I and sub-formulae thereof may exist in a number of different tautomeric forms and references to compounds of the Formula I and sub-formulae thereof include all such forms. For the avoidance of doubt, where a compound can exist in one of several tautomeric forms, and only one is specifically described or shown, all others are nevertheless embraced by Formula I and sub-formulae thereof. Examples of tautomeric forms include keto-, enol-, and enolate-forms, as in, for example, the following tautomeric pairs: keto/enol (illustrated below), pyrimidone/hydroxypyrimidine, imine/enamine, amide/imino alcohol, amidine/amidine, nitroso/oxime, thioketone/enethiol, and nitro/aci-nitro.

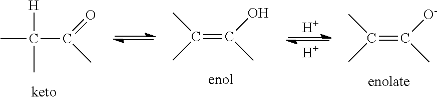

### Isomers

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

Certain compounds of Formula I and sub-formulae thereof may have one or more asymmetric centres and therefore can exist in a number of stereoisomeric configurations. Consequently, such compounds can be synthesized and/or isolated as mixtures of enantiomers and/or as individual (pure) enantiomers, and, in the case of two or more asymmetric centres, single diastereomers and/or mixtures of diastereomers. It should be understood that the present application includes all such enantiomers and diastereomers and mixtures thereof in all ratios.

### Isotopes

The compounds of the present invention are described herein using structural formulas that do not specifically recite the mass numbers or the isotope ratios of the constituent atoms. As such it is intended that the present application includes compounds in which the constituent atoms are present in any ratio of isotope forms. For example, carbon atoms may be present in any ratio of ¹²C, ¹³C, and ¹⁴C; hydrogen atoms may be present in any ratio of ¹H, ²H, and ³H; etc. Preferably, the constituent atoms in the compounds of the present invention are present in their naturally occurring ratios of isotope forms.

### Prodrugs and Metabolites

The compounds of Formula I and sub-formulae thereof may be administered in the form of a pro-drug which is broken down in the human or animal body to release a compound of the invention. A pro-drug may be used to alter the physical properties and/or the pharmacokinetic properties of a compound of the invention. A pro-drug can be formed when the compound of the invention contains a suitable group or substituent to which a property-modifying group can be attached. Examples of pro-drugs include in vivo cleavable ester derivatives that may be formed at a carboxy group or a hydroxy group in a compound of the Formula I and in-vivo cleavable amide derivatives that may be formed at a carboxy group or an amino group in a compound of the Formula I and sub-formulae thereof.

Accordingly, the present invention includes those compounds of the Formula I and sub-formulae thereof as defined hereinbefore when made available by organic synthesis and when made available within the human or animal body by way of cleavage of a pro-drug thereof. Accordingly, the present invention includes those compounds of the Formula I that are produced by organic synthetic means and also such compounds that are produced in the human or animal body byway of metabolism of a precursor compound, that is a compound of the Formula I and sub-formulae thereof may be a synthetically-produced compound or a metabolically-produced compound.

A suitable pharmaceutically acceptable pro-drug of a compound of the Formula I and sub-formulae thereof is one that is based on reasonable medical judgement as being suitable for administration to the human or animal body without undesirable pharmacological activities and without undue toxicity.

Various forms of pro-drug have been described, for example in the following documents:—
a) Methods in Enzymology, Vol. 42, p. 309-396, edited by K. Widder, et al. (Academic Press, 1985);
b) Design of Pro-drugs, edited by H. Bundgaard, (Elsevier, 1985);
c) A Textbook of Drug Design and Development, edited by Krogsgaard-Larsen and H. Bundgaard, Chapter 5 “Design and Application of Pro-drugs”, by H. Bundgaard p. 113-191 (1991);
d) H. Bundgaard, Advanced Drug Delivery Reviews, 8, 1-38 (1992);
e) H. Bundgaard, et al., Journal of Pharmaceutical Sciences, 77, 285 (1988);
f) N. Kakeya, et al., Chem. Pharm. Bull., 32, 692 (1984);
g) T. Higuchi and V. Stella, “Pro-Drugs as Novel Delivery Systems”, A.C.S. Symposium Series, Volume 14; and
h) E. Roche (editor), “Bioreversible Carriers in Drug Design”, Pergamon Press, 1987.

A suitable pharmaceutically acceptable pro-drug of a compound of the Formula I and sub-formulae thereof that possesses a carboxy group is, for example, an in vivo cleavable ester thereof. An in vivo cleavable ester of a compound of the Formula I containing a carboxy group is, for example, a pharmaceutically acceptable ester which is cleaved in the human or animal body to produce the parent acid. Suitable pharmaceutically acceptable esters for carboxy include C₁₋₆ alkyl esters such as, but not limited to, methyl, ethyl and tert-butyl, C₁₋₆ alkoxymethyl esters such as, but not limited to, methoxymethyl esters, C₁₋₆ alkanoyloxymethyl esters such as, but not limited to, pivaloyloxymethyl esters, 3-phthalidyl esters, C₃₋₈cycloalkylcarbonyloxy-C₁₋₆ alkyl esters such as, but not limited to, cyclopentylcarbonyloxymethyl and 1-cyclohexylcarbonyloxyethyl esters, 2-oxo-1,3-dioxolenylmethyl esters such as, but not limited to, 5-methyl-2-oxo-1,3-dioxolen-4-ylmethyl esters and C₁₋₈ alkoxycarbonyloxy-C₁₋₈ alkyl esters such as, but not limited to, methoxycarbonyloxymethyl and 1-methoxycarbonyloxyethyl esters.

A suitable pharmaceutically acceptable pro-drug of a compound of the Formula I and sub-formulae thereof that possesses a hydroxy group is, for example, an in vivo cleavable ester or ether thereof. An in vivo cleavable ester or ether of a compound of the Formula I and sub-formulae thereof containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as, but not limited to, phosphate esters (including phosphoramidic cyclic esters). Further suitable pharmaceutically acceptable ester forming groups for a hydroxy group include C₁₋₁₀alkanoyl groups such as, but not limited to, acetyl, benzoyl, phenylacetyl and substituted benzoyl and phenylacetyl groups, C₁₋₁₀ alkoxycarbonyl groups such as, but not limited to, ethoxycarbonyl, N,N-(C₁₋₆)₂ carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁₋₄ alkyl)piperazin-1-ylmethyl. Suitable pharmaceutically acceptable ether forming groups for a hydroxy group include α-acyloxyalkyl groups such as, but not limited to, acetoxymethyl and pivaloyloxymethyl groups.

A suitable pharmaceutically acceptable pro-drug of a compound of the Formula I and sub-formulae thereof that possesses a carboxy group is, for example, an in vivo cleavable amide thereof, for example an amide formed with an amine such as, but not limited to, ammonia, a C₁₋₄ alkylamine such as, but not limited to, methylamine, a (C₁₋₄ alkyl)₂ amine such as, but not limited to, dimethylamine, N-ethyl-N-methylamine or diethylamine, a C₁₋₄ alkoxy-C₂₋₄ alkylamine such as, but not limited to, 2-methoxyethylamine, a phenyl-C₁₋₄ alkylamine such as, but not limited to, benzylamine and amino acids such as, but not limited to, glycine or an ester thereof.

A suitable pharmaceutically acceptable pro-drug of a compound of the Formula I and sub-formulae thereof that possesses an amino group is, for example, an in vivo cleavable amide derivative thereof. Suitable pharmaceutically acceptable amides from an amino group include, for example an amide formed with C₁₋₁₀alkanoyl groups such as, but not limited to, an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁₄ alkyl)piperazin-1-ylmethyl.

The in vivo effects of a compound of the Formula I and sub-formulae thereof may be exerted in part by one or more metabolites that are formed within the human or animal body after administration of a compound of the Formula I and sub-formulae thereof. As stated hereinbefore, the in vivo effects of a compound of the Formula I and sub-formulae thereof may also be exerted by way of metabolism of a precursor compound (a pro-drug).

### Pharmaceutical Compositions

According to a further aspect of the invention there is provided a pharmaceutical composition which comprises a compound of the invention as defined hereinbefore, or a pharmaceutically acceptable salt, hydrate or solvate thereof, in association with a pharmaceutically acceptable diluent or carrier.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

An effective amount of a compound of the present invention for use in therapy is an amount sufficient to treat or prevent a proliferative condition referred to herein, slow its progression and/or reduce the symptoms associated with the condition.

The amount of active ingredient that is combined with one or more excipients to produce a single dosage form will necessarily vary depending upon the individual treated and the particular route of administration. For example, a formulation intended for oral administration to humans will generally contain, for example, from 0.5 mg to 1.5 g of active agent (more suitably from 0.5 to 600 mg, for example from 1 to 200 mg) compounded with an appropriate and convenient amount of excipients which may vary from about 5 to about 98 percent by weight of the total composition.

The size of the dose for therapeutic or prophylactic purposes of a compound of the Formula I will naturally vary according to the nature and severity of the conditions, the age and sex of the animal or patient and the route of administration, according to well-known principles of medicine.

It is to be noted that dosages and dosing regimens may vary with the type and severity of the condition to be alleviated, and may include the administration of single or multiple doses, i.e. QD (once daily), BID (twice daily), etc., over a particular period of time (days or hours). It is to be further understood that for any particular subject or patient, specific dosage regimens may need to be adjusted over time according to the individual need and the professional judgment of the person administering or supervising the administration of the pharmaceutical compositions. For example, doses may be adjusted based on pharmacokinetic or pharmacodynamic parameters, which may include clinical effects such as toxic effects and/or laboratory values. Thus, the present application encompasses intra-patient dose-escalation as determined by the person skilled in the art. Procedures and processes for determining the appropriate dosage(s) and dosing regimen(s) are well-known in the relevant art and would readily be ascertained by the skilled artisan. As such, one of ordinary skill would readily appreciate and recognize that the dosage ranges set forth herein are exemplary only and are not intended to limit the scope or practice of the pharmaceutical compositions described herein.

In using a compound of the invention for therapeutic or prophylactic purposes it will generally be administered so that a daily dose in the range, for example, 0.1 mg/kg to 75 mg/kg body weight is received, given if required in divided doses. In general lower doses will be administered when a parenteral route is employed. Thus, for example, for intravenous or intraperitoneal administration, a dose in the range, for example, 0.1 mg/kg to 30 mg/kg body weight will generally be used. Similarly, for administration by inhalation, a dose in the range, for example, 0.05 mg/kg to 25 mg/kg body weight will be used.

For the compounds of the present invention, oral administration is particularly suitable. The compounds of the present invention may be formulated as a tablet, capsule or solution for oral administration. Suitably, the compound of the present invention is formulated in a unit dosage form (e.g. a tablet or capsule) for oral administration. Typically, unit dosage forms will contain about 0.5 mg to 1.5 g of a compound of this invention.

### Synthesis

The compounds of the present invention can be prepared by any suitable technique known in the art. Particular methods for forming compounds of formula I defined herein are shown below and in the accompanying examples.

In the description of the synthetic methods described herein and in any referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilised.

It will be appreciated that during the synthesis of the compounds of the invention in the processes defined herein, or during the synthesis of certain starting materials, it may be desirable to protect certain substituent groups to prevent their undesired reaction. The skilled chemist will appreciate when such protection is required, and how such protecting groups may be put in place, and later removed.

For Examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule.

Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as, but not limited to, acetyl, an alkoxycarbonyl group, for example a methoxycarbonyl, ethoxycarbonyl or tbutoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as, but not limited to, an alkali metal hydroxide, for example lithium or sodium hydroxide. Alternatively an acyl group such as a tertbutoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium on carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or ammonia. Alternatively, an arylmethyl group such as a benzyl group may be removed, for example, by hydrogenation over a catalyst such as palladium on carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium on carbon.

Resins may also be used as a protecting group.

The methodology employed to synthesise a compound of formula (I) will vary depending on the nature of R₁, R₂, R₃ and Q₁ and any substituent groups associated therewith.

Suitable processes for their preparation are described further in the accompanying Examples.

Once a compound of formula (I) has been synthesised by any one of the processes defined herein, the processes may then further comprise one or more of the additional steps of:
(i) removing any residual protecting groups present;
(ii) converting the compound formula (I) into another compound of formula (I);
(iii) forming a pharmaceutically acceptable salt, hydrate or solvate of the compound of formula I; and/or
(iv) forming a prodrug of the compound of formula I.

An Example of (ii) above is when a compound of formula (I) is synthesised and then one or more of the groups of R₁, R₂, R₃ and Q, may be further reacted to change the nature of the group and provide an alternative compound of formula (I).

The resultant compounds of formula (I) can be isolated and purified using techniques well known in the art.

### Therapeutic Uses and Applications

The compounds of the present invention are inhibitors of EGFR, including mutated forms of EGFR that are resistant to third generations EGFR inhibitors, such as osimertinib and lazertinib. Data showing the EGFR inhibitory activity of the exemplified compounds is presented in the accompanying example section.

Accordingly, the compounds of formula I are useful for the treatment and/or prevention of diseases and conditions in which EGFR activity, including mutated forms of EGFR, is implicated, such as, for example, but not limited to, the treatment and/or prevention of proliferative disorders (e.g. cancer).

Therefore, in one aspect, the present invention provides a compound of Formula I as defined herein, or a pharmaceutically acceptable salt, hydrate or solvate thereof, or a pharmaceutical composition as defined herein, for use in therapy.

In another aspect, the present invention provides a compound of Formula I as defined herein, or a pharmaceutically acceptable salt, hydrate or solvate thereof, or a pharmaceutical composition as defined herein, for use in the treatment of a disease or condition in which EGFR activity is implicated.

In another aspect, the present invention provides a compound of Formula I as defined herein, or a pharmaceutically acceptable salt, hydrate or solvate thereof, or a pharmaceutical composition as defined herein, for use in the treatment of a disease or condition associated with aberrant activity of EGFR.

In another aspect, the present invention provides a compound of Formula I as defined herein, or a pharmaceutically acceptable salt, hydrate or solvate thereof, or a pharmaceutical composition as defined herein, for use in the treatment of proliferative disorders (e.g. cancer).

In another aspect, the present invention provides a compound of Formula I as defined herein, or a pharmaceutically acceptable salt, hydrate or solvate thereof, or a pharmaceutical composition as defined herein, for use in the treatment of cancer.

In another aspect, the present invention provides the use of a compound of Formula I as defined herein, or a pharmaceutically acceptable salt, hydrate or solvate thereof, in the manufacture of a medicament for use in the treatment of a disease or condition in which EGFR activity is implicated.

In another aspect, the present invention provides the use of a compound of Formula I as defined herein, or a pharmaceutically acceptable salt, hydrate or solvate thereof, in the manufacture of a medicament for use in the treatment of a disease or condition associated with aberrant activity of EGFR.

In another aspect, the present invention provides the use of a compound of Formula I as defined herein, or a pharmaceutically acceptable salt, hydrate or solvate thereof, in the manufacture of a medicament for use in the treatment of proliferative disorders (e.g. cancer or benign neoplasms).

In another aspect, the present invention the use of a compound of Formula I as defined herein, or a pharmaceutically acceptable salt, hydrate or solvate thereof, in the manufacture of a medicament for use in the treatment of a cancer.

In another aspect, the present invention provides a method of treating a disease or condition in which EGFR activity is implicated, said method comprising administering to a subject in need thereof an effective amount of a compound of Formula I as defined herein, or a pharmaceutically acceptable salt, hydrate or solvate thereof, or a pharmaceutical composition as defined herein.

In another aspect, the present invention provides a method of treating a disease or condition associated with aberrant activity of EGFR, said method comprising administering to a subject in need thereof an effective amount of a compound of Formula I as defined herein, or a pharmaceutically acceptable salt, hydrate or solvate thereof, or a pharmaceutical composition as defined herein.

In another aspect, the present invention provides a method of treating a proliferative disorder (e.g. cancer or benign neoplasms), said method comprising administering to a subject in need thereof an effective amount of a compound of Formula I as defined herein, or a pharmaceutically acceptable salt, hydrate or solvate thereof, or a pharmaceutical composition as defined herein.

In another aspect, the present invention provides a method of treating cancer, said method comprising administering to a subject in need thereof an effective amount of a compound of Formula I as defined herein, or a pharmaceutically acceptable salt, hydrate or solvate thereof, or a pharmaceutical composition as defined herein.

In another aspect, the present invention provides a compound of formula I, or a pharmaceutically acceptable salt, hydrate or solvate thereof, for use in the treatment of an EGFR positive cancer, optionally selected from head and neck cancer, brain cancer, breast cancer, colon cancer and/or lung cancer.

In another aspect, the present invention provides a compound of formula I, or a pharmaceutically acceptable salt, hydrate or solvate thereof, for use in the treatment of EGFR positive non-small cell lung cancer.

In another aspect, the present invention provides a compound of formula I, or a pharmaceutically acceptable salt, hydrate or solvate thereof, for use in the treatment of a cancer expressing a mutated form of EGFR (for example EGFR comprising a T790M mutation, a deletion in exon 19 (such as A740-A750), an exon 20 insertion, a mutation at L858R and/or a C797S mutation).

In another aspect, the present invention provides a compound of formula I or a pharmaceutically acceptable salt, hydrate or solvate thereof, for use in the treatment of non-small cell lung cancer expressing a mutated form of EGFR (for example EGFR comprising a T790M mutation, a deletion in exon 19 (such as A740-A750), an exon 20 insertion, a mutation at L858R and/or a C797S mutation).

In another aspect, the present invention provides a compound of formula I, or a pharmaceutically acceptable salt, hydrate or solvate thereof, for use in the treatment of a cancer resistant to treatment with a third generation EFGR inhibitor, e.g. osimertinib, lazertinib (YH25448), EGF816, olmutinib, PF-06747775, avitinib and/or rociletinib.

In another aspect, the present invention provides a compound of formula I, or a pharmaceutically acceptable salt, hydrate or solvate thereof, for use in the treatment of non-small cell lung cancer resistant to treatment with osimertinib.

In another aspect, the present invention provides the use of a compound of formula I, or a pharmaceutically acceptable salt, hydrate or solvate thereof, in the manufacture of a medicament for use in the treatment of an EGFR positive cancer, optionally selected from head and neck cancer, brain cancer, breast cancer, colon cancer and/or lung cancer.

In another aspect, the present invention provides the use of a compound of formula I, or a pharmaceutically acceptable salt, hydrate or solvate thereof, in the manufacture of a medicament for use in the treatment of EGFR positive non-small cell lung cancer.

In another aspect, the present invention provides the use of a compound of formula I, or a pharmaceutically acceptable salt, hydrate or solvate thereof, in the manufacture of a medicament for use in the treatment of a cancer expressing a mutated form of EGFR (for example EGFR comprising a T790M mutation, a deletion in exon 19 (such as A740-A750), an exon 20 insertion, a mutation at L858R and/or a C797S mutation).

In another aspect, the present invention provides the use of a compound of formula I or a pharmaceutically acceptable salt, hydrate or solvate thereof, in the manufacture of a medicament for use in the treatment of non-small cell lung cancer expressing a mutated form of EGFR (for example EGFR comprising a T790M mutation, a deletion in exon 19 (such as A740-A750), an exon 20 insertion, a mutation at L858R and/or a C797S mutation).

In another aspect, the present invention provides the use of a compound of formula I, or a pharmaceutically acceptable salt, hydrate or solvate thereof, in the manufacture of a medicament for use in the treatment of a cancer resistant to treatment with a third generation EFGR inhibitor, e.g. osimertinib, lazertinib (YH25448), EGF816, olmutinib, PF-06747775, avitinib and/or rociletinib.

In another aspect, the present invention provides the use of a compound of formula I, or a pharmaceutically acceptable salt, hydrate or solvate thereof, in the manufacture of a medicament for use in the treatment of non-small cell lung cancer resistant to treatment with osimertinib.

In another aspect, the present invention provides a method of treating an EGFR positive cancer, optionally selected from head and neck cancer, brain cancer, breast cancer, colon cancer and/or lung cancer, said method comprising administering to a subject in need thereof an effective amount of a compound of Formula I as defined herein, or a pharmaceutically acceptable salt, hydrate or solvate thereof, or a pharmaceutical composition as defined herein.

In another aspect, the present invention provides a method of treating EGFR positive non-small cell lung cancer, said method comprising administering to a subject in need thereof an effective amount of a compound of Formula I as defined herein, or a pharmaceutically acceptable salt, hydrate or solvate thereof, or a pharmaceutical composition as defined herein.

In another aspect, the present invention provides a method of treating a cancer expressing a mutated form of EGFR (for example EGFR comprising a T790M mutation, a deletion in exon 19 (such as A740-A750), an exon 20 insertion, a mutation at L858R and/or a C797S mutation), said method comprising administering to a subject in need thereof an effective amount of a compound of Formula I as defined herein, or a pharmaceutically acceptable salt, hydrate or solvate thereof, or a pharmaceutical composition as defined herein.

In another aspect, the present invention provides a method of treating non-small cell lung cancer expressing a mutated form of EGFR (for example EGFR comprising a T790M mutation, a deletion in exon 19 (such as A740-A750), an exon 20 insertion, a mutation at L858R and/or a C797S mutation), said method comprising administering to a subject in need thereof an effective amount of a compound of Formula I as defined herein, or a pharmaceutically acceptable salt, hydrate or solvate thereof, or a pharmaceutical composition as defined herein.

In another aspect, the present invention provides a method of treating a cancer resistant to treatment with a third generation EFGR inhibitor, e.g. osimertinib, lazertinib (YH25448), EGF816, olmutinib, PF-06747775, avitinib and/or rociletinib, said method comprising administering to a subject in need thereof an effective amount of a compound of Formula I as defined herein, or a pharmaceutically acceptable salt, hydrate or solvate thereof, or a pharmaceutical composition as defined herein.

In another aspect, the present invention provides a method of treating non-small cell lung cancer resistant to treatment with osimertinib, said method comprising administering to a subject in need thereof an effective amount of a compound of Formula I as defined herein, or a pharmaceutically acceptable salt, hydrate or solvate thereof, or a pharmaceutical composition as defined herein.

The terms “proliferative disorder” and “proliferative condition” are used interchangeably herein and pertain to an unwanted or uncontrolled cellular proliferation of excessive or abnormal cells which is undesired, such as, neoplastic or hyperplastic growth, whether in vitro or in vivo.

Examples of proliferative conditions include, but are not limited to, pre-malignant and malignant cellular proliferation, including but not limited to, cancers, psoriasis, bone diseases, fibroproliferative disorders (e.g. of connective tissues), and atherosclerosis. Any type of cell may be treated.

Cancers associated with aberrant EGFR activity (including its mutated forms—for example EGFR comprising a T790M mutation, a deletion in exon 19 (such as A740-A750), an exon 20 insertion, a mutation at L858R and/or a C797S mutation) are of particular interest. Thus, the compounds of the present invention may be used to treat any EGFR positive cancer. Particular examples of such cancers include head and neck cancer, brain cancer, breast cancer, colon cancer and/or lung cancer. A particular cancer of interest is non-small cell lung cancer.

Thus, in certain aspects of the present invention, the proliferative disorder is cancer, suitably a cancer selected from head and neck cancer, brain cancer, breast cancer, colon cancer and/or lung cancer.

In a particular aspect of the invention, the proliferative disorder is non-small cell lung cancer.

It will be appreciated that the compounds of the present invention could be used to treat any EGFR positive cancer. The invention therefore encompasses the treatment of any EGFR positive non-metastatic or metastatic cancer and which may be a solid tumour or a haematological (“liquid”) cancer. The cancer may, for example, be selected from:
(1) Carcinoma, including for example tumours derived from stratified squamous epithelia (squamous cell carcinomas) and tumours arising within organs or glands (adenocarcinomas). Examples include breast, colon, lung, prostate, ovary, esophageal carcinoma (including, but not limited to, esophageal adenocarcinoma and squamous cell carcinoma), basal-like breast carcinoma, basal cell carcinoma (a form of skin cancer), squamous cell carcinoma (various tissues), head and neck carcinoma (including, but not limited to, squamous cell carcinomas), stomach carcinoma (including, but not limited to, stomach adenocarcinoma, gastrointestinal stromal tumor), signet ring cell carcinoma, bladder carcinoma (including transitional cell carcinoma (a malignant neoplasm of the bladder)), bronchogenic carcinoma, colorectal carcinoma (including, but not limited to, colon carcinoma and rectal carcinoma), anal carcinoma, gastric carcinoma, lung carcinoma (including but not limited to small cell carcinoma (SCLC) and non-small cell carcinoma of the lung (NSCLC), lung adenocarcinoma, squamous cell carcinoma, large cell carcinoma, bronchioloalveolar carcinoma, and mesothelioma), neuroendocrine tumors (including but not limited to carcinoids of the gastrointestinal tract, breast, and other organs), adrenocortical carcinoma, thyroid carcinoma, pancreatic carcinoma (including, but not limited to, pancreatic ductal adenocarcinoma, pancreatic adenocarcinoma, acinar cell carcinoma, intraductal papillary mucinous neoplasm with invasive carcinoma, mucinous cystic neoplasm with invasive carcinoma, islet cell carcinoma and neuroendocrine tumors), breast carcinoma (including, but not limited to, ductal carcinoma, lobular carcinoma, inflammatory breast cancer, clear cell carcinoma, mucinous carcinoma), ovarian carcinoma (including, but not limited to, ovarian epithelial carcinoma or surface epithelial-stromal tumor including serous tumor, endometrioid tumor and mucinous cystadenocarcinoma, sex-cord-stromal tumor), liver and bile duct carcinoma (including, but not limited to, hepatocellular carcinoma, cholangiocarcinoma and hemangioma), prostate carcinoma, adenocarcinoma, brain tumours (including, but not limited to glioma, glioblastoma and medulloblastoma), germ cell tumors, sweat gland carcinoma, sebaceous gland carcinoma, papillary carcinoma, papillary adenocarcinoma, cystadenocarcinoma, kidney carcinoma (including, but not limited to, renal cell carcinoma, clear cell carcinoma and Wilm's tumor), medullary carcinoma, ductal carcinoma in situ or bile duct carcinoma, choriocarcinoma, seminoma, embryonal carcinoma, cervical carcinoma, uterine carcinoma (including, but not limited to, endometrial adenocarcinoma, uterine papillary serous carcinoma, uterine clear-cell carcinoma, uterine sarcomas and leiomyosarcomas, mixed mullerian tumors), testicular carcinoma, osteogenic carcinoma, epithelial carcinoma, sarcomatoid carcinoma, nasopharyngeal carcinoma, laryngeal carcinoma; oral and oropharyngeal squamous carcinoma;
(2) Sarcomas, including: osteosarcoma and osteogenic sarcoma (bone); chondrosarcoma (cartilage); leiomyosarcoma (smooth muscle); rhabdomyosarcoma (skeletal muscle); mesothelial sarcoma and mesothelioma (membranous lining of body cavities); fibrosarcoma (fibrous tissue); angiosarcoma and hemangioendothelioma (blood vessels); liposarcoma (adipose tissue); glioma and astrocytoma (neurogenic connective tissue found in the brain); myxosarcoma (primitive embryonic connective tissue); chordoma, endotheliosarcoma, lymphangiosarcoma, lymphangioendotheliosarcoma, synovioma, Ewing's sarcoma, mesenchymous and mixed mesodermal tumor (mixed connective tissue types) and other soft tissue sarcomas;
(3) Myeloma and multiple myeloma;
(4) Hematopoietic tumours, including: myelogenous and granulocytic leukemia (malignancy of the myeloid and granulocytic white blood cell series); lymphatic, lymphocytic, and lymphoblastic leukemia (malignancy of the lymphoid and lymphocytic blood cell series); polycythemia vera and erythremia (malignancy of various blood cell products, but with red cells predominating); myelofibrosis.
(5) Lymphomas, including: Hodgkin and Non-Hodgkin lymphomas;
(6) Solid tumors of the nervous system including medulloblastoma, craniopharyngioma, ependymoma, pinealoma, hemangioblastoma, acoustic neuroma, oligodendroglioma, meningioma, neuroblastoma and schwannoma;
(7) Melanoma, uveal melanoma and retinoblastoma; and
(8) Mixed Types, including, e.g., adenosquamous carcinoma, mixed mesodermal tumor, carcinosarcoma or teratocarcinoma.

### Routes of Administration

The compounds of the invention or pharmaceutical compositions comprising these compounds may be administered to a subject by any convenient route of administration, whether systemically/peripherally or topically (i.e., at the site of desired action).

Routes of administration include, but are not limited to, oral (e.g. by ingestion); buccal; sublingual; transdermal (e.g. by a patch, plaster, etc.); transmucosal (e.g. by a patch, plaster, etc.); intranasal (e.g. by nasal spray); ocular (e.g. by eye drops, eye ointment etc.); pulmonary (e.g. by inhalation or insufflation therapy, for example via an aerosol, for example by the nose or mouth); rectal (e.g. by suppository or enema); vaginal (e.g. by pessary); parental, for example by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir dosage form, for example subcutaneously or intramuscularly.

The compounds of the present invention are particularly suitable for oral administration.

### Combination Therapies

The compounds of the invention and salts, solvates thereof defined hereinbefore may be applied as a sole therapy or may involve, in addition to the compound of the invention, one or more additional therapeutic agents, e.g. an anti-tumour agent.

In the context of cancer treatment, in addition to the compound of the invention therapy may involve conventional surgery or radiotherapy or chemotherapy. Such chemotherapy may include one or more of the following categories of anti-tumour agents:—
other antiproliferative/antineoplastic drugs and combinations thereof, as used in medical oncology, such as, but not limited to, alkylating agents (for example cisplatin, oxaliplatin, carboplatin, cyclophosphamide, nitrogen mustard, melphalan, chlorambucil, busulphan, temozolamide and nitrosoureas); antimetabolites (for example gemcitabine and antifolates such as, but not limited to, fluoropyrimidines like 5-fluorouracil and tegafur, raltitrexed, methotrexate, cytosine arabinoside, and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecin);
cytostatic agents such as, but not limited to, antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as, but not limited to, finasteride;
anti-invasion agents [for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline (AZD0530; International Patent Application WO 01/94341), N-(2-chloro-6-methyl)phenyl)-2-{6-[4-(2-hydroxyethyl)piperazin-1-yl]-2-methylpyrimidin-4-yl]amino}thiazole-5-carboxamide (dasatinib, BMS-354825; J. Med. Chem., 2004, 47, 6658-6661) and bosutinib (SKI-606), and metalloproteinase inhibitors like marimastat, inhibitors of urokinase plasminogen activator receptor function or antibodies to Heparanase];
inhibitors of growth factor function: for example such inhibitors include growth factor antibodies and growth factor receptor antibodies (for example the anti-erbB2 antibody trastuzumab [Herceptin™], the anti-EGFR antibody panitumumab, the anti-erbB1 antibody cetuximab [Erbitux, C225] and any growth factor or growth factor receptor antibodies disclosed by Stern et al. (Critical reviews in oncology/haematology, 2005, Vol. 54, pp11-29); such inhibitors also include tyrosine kinase inhibitors, for example inhibitors of the epidermal growth factor family (for example other EGFR family tyrosine kinase inhibitors such as, but not limited to, N-(3-chloro-4-fluorophenyl)-7-methoxy-6-(3-morpholinopropoxy)quinazolin-4-amine (gefitinib, ZD1839), N-(3-ethynyl)phenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)-quinazolin-4-amine (CI 1033), afatinib, osimertinib, lazertinib (YH25448), EGF816, olmutinib, PF-06747775, avitinib, rociletinib; erbB2 tyrosine kinase inhibitors such as, but not limited to, lapatinib); inhibitors of the hepatocyte growth factor family; inhibitors of the insulin growth factor family; inhibitors of the platelet-derived growth factor family such as, but not limited to, imatinib and/or nilotinib (AMN107); inhibitors of serine/threonine kinases (for example Ras/Raf signalling inhibitors such as, but not limited to, farnesyl transferase inhibitors, for example sorafenib (BAY 43-9006), tipifarnib (R115777) and lonafarnib (SCH66336)), inhibitors of cell signalling through MEK and/or AKT kinases, c-kit inhibitors, abl kinase inhibitors, P13 kinase inhibitors, Plt3 kinase inhibitors, CSF-1R kinase inhibitors, IGF receptor (insulin-like growth factor) kinase inhibitors; aurora kinase inhibitors (for example AZD1152, PH739358, VX-680, MLN8054, R763, MP235, MP529, VX-528 AND AX39459) and cyclin dependent kinase inhibitors such as, but not limited to, CDK2 and/or CDK4 inhibitors;
antiangiogenic agents such as, but not limited to, those which inhibit the effects of vascular endothelial growth factor, [for example the anti-vascular endothelial cell growth factor antibody bevacizumab (Avastin™) and for example, a VEGF receptor tyrosine kinase inhibitor such as, but not limited to, vandetanib (ZD6474), vatalanib (PTK787), sunitinib (SU11248), axitinib (AG-013736), pazopanib (GW 786034) and 4-(4-fluoro-2-methylndol-5-yloxy)-6-methoxy-7-(3-pyrrolidin-1-ylpropoxy)quinazoline (AZD2171; Example 240 within WO 00/47212), compounds such as, but not limited to, those disclosed in International Patent Applications WO97/22596, WO 97/30035, WO 97/32856 and WO 98/13354 and compounds that work by other mechanisms (for example linomide, inhibitors of integrin avp33 function and angiostatin)];
vascular damaging agents such as, but not limited to, Combretastatin A4 and compounds disclosed in International Patent Applications WO 99/02166, WO 00/40529, WO 00/41669, WO 01/92224, WO 02/04434 and WO 02/08213;
an endothelin receptor antagonist, for example zibotentan (ZD4054) or atrasentan;
antisense therapies, for example those which are directed to the targets listed above, such as, but not limited to, ISIS 2503, an anti-ras antisense;
gene therapy approaches, including for example approaches to replace aberrant genes such as, but not limited to, aberrant p53 or aberrant BRCA1 or BRCA2, GDEPT (gene-directed enzyme pro-drug therapy) approaches such as, but not limited to, those using cytosine deaminase, thymidine kinase or a bacterial nitroreductase enzyme and approaches to increase patient tolerance to chemotherapy or radiotherapy such as multi-drug resistance gene therapy; and
immunotherapy approaches, including for example ex-vivo and in-vivo approaches to increase the immunogenicity of patient tumour cells, such as, but not limited to, transfection with cytokines such as interleukin 2, interleukin 4 or granulocyte-macrophage colony stimulating factor, approaches to decrease T-cell anergy, approaches using transfected immune cells such as, but not limited to, cytokine-transfected dendritic cells, approaches using cytokine-transfected tumour cell lines and approaches using anti-idiotypic antibodies.

In a particular embodiment, the antiproliferative treatment defined hereinbefore may involve, in addition to the compound of the invention, conventional surgery or radiotherapy or chemotherapy.

In a further particular embodiment, the antiproliferative treatment defined hereinbefore may involve, in addition to the compound of the invention, standard chemotherapy for the cancer concerned.

Such conjoint treatment may be achieved by way of the simultaneous, sequential or separate dosing of the individual components of the treatment. Such combination products employ the compounds of this invention within the dosage range described hereinbefore and the other pharmaceutically-active agent within its approved dosage range.

According to this aspect of the invention there is provided a combination for use in the treatment of a cancer (for example a cancer involving a solid tumour) comprising a compound of the invention as defined hereinbefore, or a pharmaceutically acceptable salt, hydrate or solvate thereof, and another anti-tumour agent, or a pharmaceutically acceptable salt thereof.

According to this aspect of the invention there is provided a combination for use in the treatment of a proliferative condition, such as, but not limited to, cancer (for example a cancer involving a solid tumour), comprising a compound of the invention as defined hereinbefore, or a pharmaceutically acceptable salt, hydrate or solvate thereof, and any one of the anti-tumour agents listed herein above.

In a further aspect of the invention there is provided a compound of the invention or a pharmaceutically acceptable salt, hydrate or solvate thereof, for use in the treatment of cancer in combination with another anti-tumour agent, optionally selected from one listed herein above.

Herein, where the term “combination” is used it is to be understood that this refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the beneficial effect of the combination. In one embodiment, a combination refers to a combination product.

According to a further aspect of the invention there is provided a pharmaceutical composition which comprises a compound of the invention, or a pharmaceutically acceptable salt, hydrate or solvate thereof, in combination with an anti-tumour agent (optionally selected from one listed herein above), in association with a pharmaceutically acceptable diluent or carrier.

### Biological Activity

The biological assays described in the example section may be used to measure the pharmacological effects of the compounds of the present invention. Although the pharmacological properties of the compounds of formula I vary with structural change, as expected, the compounds of the invention were found to be active in the biological assays described in the example section below.

### EXAMPLES

The invention will now be illustrated, but not limited, by reference to the specific embodiments described in the following examples. Compounds are named using conventional IUPAC nomenclature, or as named by the chemical supplier.

The following synthetic procedures are provided for illustration of the methods used; for a given preparation or step the precursor used may not necessarily derive from the individual batch synthesized according to the step in the description given.

### General Information

Chemicals were purchased from commercial suppliers and used without further purification. Thin layer chromatography (TLC) was performed on aluminum plates coated with 60 F254 silica from Merck. Flash chromatography was carried out using a Biotage SP4, Biotage Isolera, or Varian automated flash system with Silicycle or GraceResolve normal phase silica gel prepacked columns. Fractions were collected at 254 nm or if necessary, on all wavelengths between 200 and 400 nm. Microwave irradiation was performed in a Biotage Initiator Sixty in sealed vials. Reactions were irradiated at 2.45 GHz and were able to reach temperatures between 6° and 250° C. Heating was at a rate of 2-5° C./s, and the pressure was able to reach 20 bar. Final compound purity is >95%. Flash chromatography was also carried out using a Biotage SP4, Biotage Isolera Prime, Varian or Agela Technologies automated flash system with GraceResolve normal phase silica gel pre-packed columns, Welch Technology or Agela Technologies normal phase silica gel columns or reverse phase C18 columns. Fractions were collected at 254 nm or if necessary, on all wavelengths between 200 and 400 nm. Microwave irradiation was performed in a Biotage Initiator Sixty in sealed vials. Reactions were irradiated at 2.45 GHz and were able to reach temperatures between 4° and 300° C. Heating was at a rate of 2-5° C./s and the pressure was able to reach 20 bar.

### Analytical Equipment

LC-MS analyses were conducted using a Waters Acquity UPLC system or Shimadzu LCMS-2020 with photodiode array (PDA) and evaporating light scattering detector (ELSD). When a 2 min gradient was used, the sample was eluted on an Acquity UPLC BEH C18, 1.7 μm, 2.1 mm×50 mm, with a flow rate of 0.6 mL/min using 5-95% 0.1% HCOOH in MeCN or LCMS Ascentis Express 90 A C18 2.7 μm (3 cm×3.0 mm) with a flow rate of 1.2 mL/min using 5-95% 0.1% HCOOH in MeCN or Poroshell HPH—C18, 4.0 μm (5 cm×3.0 mm) with a flow rate of 1.2 ml/min using 5-95% 5 mmol NH₄HCO₃ in MeCN. The analytical purity of compounds was determined using Waters XTerra RP18, 5 μm (4.6×150 mm) column at 1 mL/min either using 0.1% aq. ammonia and MeCN or 0.1% aq. HCOOH and MeCN with a gradient of 5-100% over 15 min or Shimadzu Ascentis Express C18, 2.7 μm (4.6×100 mm) column or Agilent EVO C18, 2.6 μm (3.0×100 mm) column at 1 mL/min using either 0.05% aq. ammonia and MeCN or 0.1% aq. TFA and MeCN with a gradient of 5-100% over 15 min.

¹H NMR spectra were obtained using a Bruker Avance III 500 spectrometer using a frequency of 500 MHz, a Bruker BioSpin AG Avanace III HD using a frequency of 300 MHz or a Bruker BioSpin AG Avanace NEO using a frequency of 400 MHz. ³C spectra were acquired using the Bruker Avance III 500 spectrometer operating at a frequency of 126 MHz. The abbreviations for spin multiplicity are as follows: s=singlet; d=doublet; t=triplet; q=quartet; p=quintuplet; h=sextuplet; m=multiplet. Combinations of these abbreviations are employed to describe more complex splitting patterns (e.g., dd=doublet of doublets).

### General Procedures

### General Procedure 1

TIPS-protected alkyne (1.0 eq.) and potassium fluoride (1.0-20 eq.) in solvent (0.1 M) was stirred at the specified temperature until complete. The solvent was removed under reduced pressure.

### General Procedure 2

Methylthiol (1.0 eq.) and m-CPBA (3.0 eq.) in dichloromethane (0.1 M) were stirred at room temperature for 1-2 hours. The reaction mixture was quenched with saturated aqueous sodium thiosulphate solution (20 mL) and extracted with dichloromethane (3×30 mL). The combined organic extracts were washed with saturated aqueous sodium thiosulfate solution (3×20 mL) and saturated aqueous sodium hydrogen carbonate solution (3×30 mL). The organic layer was dried (MgSO₄) and concentrated under reduced pressure. The crude material was carried into the subsequent step without further purification unless otherwise stated.

### General Procedure 3

Methylsulfonyl (1.0 eq.), aniline (1.0-1.5 eq.) and trifluoroacetic acid (1.0-1.5 eq.) in acetonitrile or n-butanol (0.1 M) were stirred at 80-110° C. overnight. The reaction mixture was concentrated under reduced pressure.

### General Procedure 4

Bromide (1.0 eq.) and amine (1.5 eq.) and triethylamine (2.5 eq.) in dichloromethane (1M) were stirred for 2 h at room temperature under nitrogen atmosphere then concentrated under reduced pressure.

### General Procedure 5

To a solution of nitro (1.0 eq.) in ethanol (0.25 M) or ethanol:acetic acid (9:1, 0.25 M) was added palladium on carbon (0.1 eq.). The reaction mixture was stirred at room temperature overnight under an atmosphere of hydrogen. The reaction mixture was filtered through Celite and the filtrate was concentrated under reduced pressure.

### General Procedure 6

p-Fluoronitrophenyl (1.0 eq.), amine (1.0-2.0 eq.) and potassium carbonate (1.5-2.0 eq.) in DMSO (0.6-1.0 M) was stirred at room temperature overnight. The reaction mixture was poured onto water (30 mL) and the resulting precipitate collected by vacuum filtration washing with water and dried in a vacuum over overnight or extracted with dichloromethane (3×100 mL). The combined organic extracts were dried (MgSO₄) and concentrated under reduced pressure.

### General Procedure 7

To a solution of pyrido[2,3-d]pyrimidin-7-amine (1.0 eq.) in dichloromethane (0.25 M) was added triethylamine (2.0 eq.) and CDI (1.5 eq.). The mixture was stirred at room temperature until the disappearance of starting material, monitored by LCMS, and then the desired amine or alcohol (2.0 eq.) was added. The mixture was stirred at room temperature overnight then extracted with ethyl acetate (2×30 mL). The combined organic layers were washed with brine (3×50 mL), dried (Na₂SO₄) and concentrated under reduced pressure.

### General Procedure 8

Pyridone (1.0 eq.), alcohol (1.1 eq.), triphenylphosphine (1.5-2.0 eq.) and DEAD or DIAD (1.5-2.0 eq.) in solvent (0.5 M) were stirred at room temperature for 1 h. The reaction mixture was quenched with water (30 mL), extracted with dichloromethane (3×30 mL). The combined organic extracts were dried (MgSO₄) and concentrated under reduced pressure.

### Example 1

### N²-(2-methoxyphenyl)pyrimidine-2,4-diamine

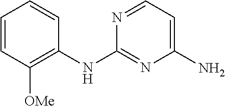 [SMILES: COc1ccccc1Nc1nccc(N)n1]

A mixture solution of 2-chloropyrimidin-4-amine (5.00 g, 38.6 mmol, 1.0 eq.) and 2-methoxyaniline (5.70 g, 46.3 mmol, 1.2 eq.) in n-butanol (100 mL) was stirred overnight at 115° C. The mixture was cooled to room temperature, diluted with water (150 mL) and extracted with ethyl acetate (3×250 mL). The combined organic layers were dried (Na₂SO₄) and concentrated under reduced pressure. The residue was purified by flash column chromatography, eluting with methanol (8%) in dichloromethane to afford the title compound as a brown solid (8.10 g, 97%).

### 5-[(1E)-({2-[(2-methoxyphenyl)amino]pyrimidin-4-yl}imino)methyl]-2,2-dimethyl-1,3

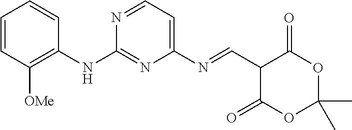 [SMILES: COc1ccccc1Nc1nccc(/N=C/C2C(=O)OC(C)(C)OC2=O)n1]

A solution of N²-(2-methoxyphenyl)pyrimidine-2,4-diamine (8.10 g, 37.5 mmol, 1.0 eq.) and 5-(methoxymethyldene)-2,2-dimethyl-1,3-dioxane-4,6-dione (10.5 g, 56.2 mmol, 1.5 eq.) in methanol (150 mL) was stirred overnight at 65° C. The mixture was cooled to room temperature. The precipitated solids were collected by filtration, washing with hexane (2×25 mL) to yield the title compound as a light brown solid (5.00 g, 36%).

### 2-[(2-methoxyphenyl)amino]-8H-pyrido[2,3-d]pyrimidin-5-one

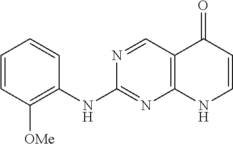 [SMILES: COc1ccccc1Nc1ncc2c(=O)cc[nH]c2n1]

A solution of 5-[(1E)-({2-[(2-methoxyphenyl)amino]pyrimidin-4-yl}imino)methyl]-2,2-dimethyl-1,3-dioxane-4,6-dione (5.00 g, 13.5 mmol, 1.0 eq.) in phenoxybenzene (50 mL) was stirred for 10 minutes at 250° C. The mixture was cooled to room temperature and diluted with water (100 mL). The precipitated solids were collected by filtration, washing with ethyl acetate (2×5 mL) to yield the title compound as a white solid (3.50 mg, 96%).

### N-(2-methoxyphenyl)-5-[2-(triisopropylsilyl) ethynyl]pyrido[2,3-d]pyrimidin-2-amine

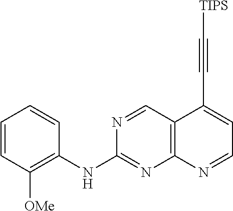

A solution of 2-[(2-methoxyphenyl)amino]-8H-pyrido[2,3-d]pyrimidin-5-one (500 mg, 1.0 eq.), phosphoryl bromide (643 mg, 1.2 eq.) in DMF (10.00 mL), and triethylamine (227 mg, 1.20 eq.) was stirred for 1 h at 90° C. After cooling to room temperature, N,N-diisopropyl-N-ethylamine (5.0 mL), bis(triphenylphosphine)palladium (II) chloride (134 mg, 0.1 eq.), copper iodide (37 mg, 0.1 eq.), triisopropylsilylacetylene (681 mg, 2 eq.) and DMF (5.00 mL) were added. The resulting solution was stirred for 2 h at 80° C. then cooled to room temperature.

The reaction was quenched by the addition of water (10 mL) and diluted with dichloromethane (20 mL). The resulting solution was extracted with dichloromethane (2×100 mL) and the combined organic layers were dried (Na₂CO₃) and concentrated under reduced pressure. The residue was purified by flash column chromatography eluting with ethyl acetate (30%) in 40-60 petroleum ether and further purified by reverse phase flash column chromatography eluting with acetonitrile (75-100%) in water (0.1% NH₄HCO₃) to yield the title compound as a brown solid (48.0 mg, 5.9%).

(ES, m/z): [M+1]⁺=433

### 5-ethynyl-N-(2-methoxyphenyl)pyrido[2,3-d]pyrimidin-2-amine

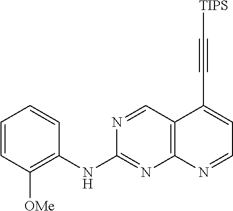

General Procedure 1 was applied to N-(2-methoxyphenyl)-5-[2-(triisopropylsilyl)ethynyl]pyrido[2,3-d]pyrimidin-2-amine (40 mg, 1.0 eq.) and potassium fluoride (55 mg, 10 eq.) in THF, methanol and water (V/V/V 2:1:1, 10 mL). The resulting solution was stirred for 6 h at 40° C. then diluted with water (5 mL) and extracted with ethyl acetate (3×20 mL). The combined organic layers were dried (Na₂SO₄) and concentrated under reduced pressure. The residue was purified by reverse phase flash column chromatography, and the desired fractions concentrated under reduced pressure to remove most of the acetonitrile, then lyophilizied to dryness to yield the title compound as a brown solid (25 mg, 99%).

(ES, m/z): [M+1]*=277 ¹H NMR (300 MHz, Chloroform-d) δ 9.50 (s, 1H), 9.01 (d, J=4.6 Hz, 2H), 8.29 (s, 1H), 7.36 (d, J=4.7 Hz, 1H), 7.13-7.04 (m, 2H), 6.96 (d, J=9.5 Hz, 1H), 3.97 (s, 3H), 3.75 (s, 1H).

### Example 2

Ethyl 4-acetamido-2-(methylthio)pyrimidine-5-carboxylate

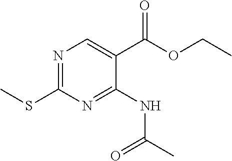 [SMILES: CCOC(=O)c1cnc(SC)nc1NC(C)=O]

A solution of ethyl 4-amino-2-(methylthiol)pyrimdine-5-carboxylate (2.00 g, 9.38 mmol, 1.0 eq.) in acetic anhydride (15 mL, 0.6 M) was subjected to microwave heating at 150° C. for 20 minutes then 30 minutes. The reaction mixture was concentrated under reduced pressure then dissolved in dichloromethane and filtered through SCX to remove any unreacted starting material. The filtrate was concentrated under reduced pressure and the crude material was carried forward without any further purification (2.22 g, 8.67 mmol, 92%).

(ES, m/z): [M+1]⁺=256.1

### 5-hydroxy-2-(methylthio)pyrido[2,3-d]pyrimidin-7(8H)-one

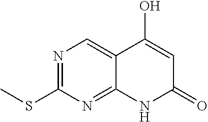

To a stirred solution of ethyl 4-acetamido-2-(methylthio)pyrimidine-5-carboxylate (1.00 g, 3.92 mmol, 1.0 eq.) in THF (39 mL, 0.1 M) at 0° C. was added KHMDS (1.0 M sol. in THF, 11.7 mL, 11.7 mmol, 3.0 eq.) dropwise. The reaction mixture was stirred at 0° C. for 30 minutes, then quenched with methanol and concentrated under reduced pressure. The crude material was suspended in water and acidifed with 6M aqueous HCl. The resulting precipitate was collected by vacuum filtration and dried in the vacuum oven overnight. The crude material was carried forward without any further purification (568 mg, 2.71 mmol, 69%).

(ES, m/z): [M+1]⁺=210.0

### 2-(methylthio)-5-((triisopropylsilyl)ethynyl)pyrido[-2.3-d]pyrimidin-7(8H)-one

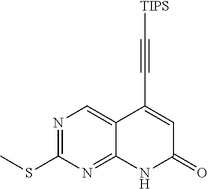

A solution of 5-hydroxy-2-(methylthio)pyrido[2,3-d]pyrimidin-7(8H)-one (6.00 g, 28.6 mmol, 1.0 eq.) and N,N-phenyl bistrifluoromethane sulfonamide (12.3 g, 36.4 mmol, 1.1 eq.) in THF (114 mL, 0.25 M) was sparged with nitrogen and then triethylamine (8.00 mL, 57.4 mmol, 2.0 eq.) was added. The reaction mixture was stirred at 60° C. for 90 mins then copper iodide (546 mg, 2.86 mmol, 0.1 eq.), bis(triphenylphosphine) palladium (II) chloride dichloromethane (2.10 g, 2.86 mmol, 0.1 eq.) and TIPS-acetylene (12.9 mL, 57.4 mmol, 2.0 eq.) and then stirring continued at 60° C. overnight. The reaction mixture was concentrated under reduced pressure then dissolved in dichloromethane (100 mL) and washed with water (100 mL). The aqueous layer was extracted with dichloromethane (5×50 mL) and the combined organic layers were dried (MgSO₄) and concentrated under reduced pressure. The crude material was purified by flash column chromatography eluting with ethyl acetate (10-30%) in 40-60 petroleum ether to yield the title compound as a pale yellow solid (3.38 g, 9.06 mmol, 32%).

(ES, m/z): [M+1]*=374.4 7-chloro-2-(methylthio)-5-((triisopropylsilyl)ethynyl)pyrido[2,3-d]pyrimidine

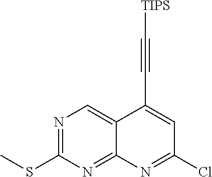

A solution of 2-(methylsulfanyl)-5-[2-(triisopropylsilyl)ethynyl]-8H-pyrido[2,3-d]pyrimidin-7-one (1.00 g, 2.68 mmol, 1.0 eq.) in phosphorus oxychloride (9.00 mL, 0.3 M) was stirred for 2 h at 90° C. The resulting mixture was concentrated under reduced pressure.

The residue was quenched with water (100 mL), saturated sodium hydrogen carbonate solution (50 mL) and dichloromethane (100 mL). The layers were separated and the aqueous phase further extracted with dichloromethane (3×50 mL). The combined organic layers were dried (MgSO₄) and concentrated under reduced pressure. The residue was purified by flash column chromatography eluting with ethyl acetate (4%) in 40-60 petroleum ether to afford the title compound as a colourless oil (620 mg, 59%).

(ES, m/z): [M+H]⁺=392.1.

### 7-methoxy-2-(methylthio)-5-((triisopropylsilyl)ethynyl)pyrido[2,3-d]pyrimidine

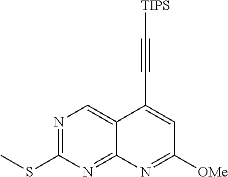

To a solution of 7-chloro-2-(methylthio)-5-((triisopropylsilyl)ethynyl)pyrido[2,3-d]pyrimidine (105 mg, 0.268 mmol, 1.0 eq.) in methanol (3 mL) was added sodium methoxide (25% in methanol, 0.3 mL, 1.34 mmol, 5.0 eq.). The mixture was stirred at room temperature for 1.5 h then concentrated under reduced pressure. The residue was suspended in water and extracted with dichloromethane (3×30 mL). The combined organic extracts were dried (MgSO₄) and concentrated under reduced pressure to yield the title compound as a brown solid (93% over two steps). (ES, m/z): [M+1]*=388.3 ¹H NMR (500 MHz, CDC₃): δ_{H}=1.07 (3H, s), 1.09 (18H, s), 2.66 (3H, s), 4.10 (3H, s), 6.93 (1H, s), 9.26 (1H, s) N¹-(2-(dimethylamino)ethyl)-N⁴-(7-methoxy-5-((triisopropylsilyl)ethynyl)pyrido[2,3-d]pyrimidin-2-yl)-N¹-methylbenzene-1,4-diamine

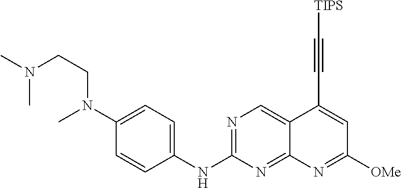

General Procedure 2 was applied to 7-methoxy-2-(methylthio)-5-((triisopropylsilyl)ethynyl)pyrido[2,3-d]pyrimidine (550 mg, 1.42 mmol) with m-CPBA (952 mg, 5.53 mmol) in dichloromethane (27.5 mL). The crude material was concentrated under reduced pressure to yield 7-methoxy-2-(methylsulfonyl)-5-((triisopropylsilyl)ethynyl)pyrido[2,3-d]pyrimidine as a yellow oil (590 mg, quantitative yield).

General Procedure 3 was applied to 7-methoxy-2-(methylsulfonyl)-5-((triisopropylsilyl)ethynyl)pyrido[2,3-d]pyrimidine (250 mg, 0.59 mmol) with N¹,N¹,N²-trimethyl-N²-(4-nitrophenyl)ethane-1,2-diamine (172 mg, 0.885 mmol) and trifluoroacetic acid (71.0 μL, 0.885 mmol) in acetonitrile (6 mL). The crude material was purified by flash column chromatography eluting with methanol (10%) in dichloromethane to yield the title compound as an orange solid (130 mg, 40% over 2 steps). (ES, m/z): [M+1]*=534.5 ¹H NMR (500 MHz, CDCl3): δ_{H}=1.15 (3H, s), 1.16 (18H, s), 2.43 (6H, s), 2.65 (2H, t, J=7.1 Hz), 2.97 (3H, s), 3.56 (2H, t, J=7.1 Hz), 4.11 (3H, s), 6.76 (m, 3H), 7.21 (s, 1H), 7.48 (2H, d, J=8.5 Hz), 9.22 (1H, s) N¹-(2-(dimethylamino)ethyl)-N⁴-(5-ethynyl-7-methoxypyrido[2,3-d]pyrimidin-2-yl)-N¹-methylbenzene-1,4-diamine

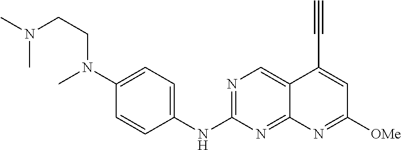 [SMILES: C#Cc1cc(OC)nc2nc(Nc3ccc(N(C)CCN(C)C)cc3)ncc12]

General Procedure 1 was applied to N¹-(2-(dimethylamino)ethyl)-N⁴-(7-methoxy-5-((triisopropylsilyl)ethynyl)pyrido[2,3-d]pyrimidin-2-yl)-N¹-methylbenzene-1,4-diamine (130 mg, 0.24 mmol) with potassium fluoride (280 mg, 4.80 mmol) in DMF (5 mL). The crude material was purified by flash chromatography eluting with methanol (10%) in dichloromethane to yield the title compound as an orange solid the title compound (40 mg, 44%)

(ES, m/z): [M+1]⁺=377.3

¹H NMR (500 MHz, CDCl₃): H=9.19 (1H, s), 7.48 (2H, d, J=8.3 Hz), 7.21 (1H, s), 6.78 (1H, s), 6.76 (2H, d, J=9.1 Hz) 4.11 (3H, s), 3.58 (1H, s), 3.51 (2H, t, J=7.3 Hz), 2.97 (3H, s), 2.58 (2H, t, J=7.3 Hz), 2.37 (6H, s)

### Example 3

### 7-methoxy-N-(4-(4-methylpiperazin-1-yl)phenyl)-5-((triisopropylsilyl)ethynyl)pyrido[2,3-d]pyrimidin-2-amine

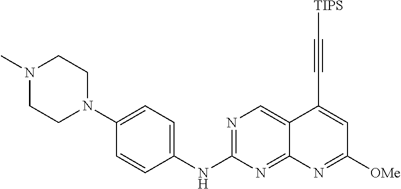

General Procedure 3 was applied to 7-methoxy-2-(methylsulfonyl)-5-((triisopropylsilyl)ethynyl)pyrido[2,3-d]pyrimidine (140 mg, 0.334 mmol) with 4-(4-methylpiperazin-1-yl)aniline (96.0 mg, 0.500 mmol) and trifluoroacetic acid (40.0 μL, 0.500 mmol) in acetonitrile (3.3 mL). The crude material was purified by flash column chromatography eluting with methanol (10%) in dichloromethane to yield the title compound as an orange solid (90 mg, 51%).

(ES, m/z): [M+H]⁺=531.5

¹H NMR (500 MHz, CDCl3): δ_{H}=1.06 (3H, s), 1.08 (18H, s), 2.37 (3H, s), 2.67 (4H, t, J=4.2 Hz), 3.17 (4H, t, J=4.2 Hz), 3.99 (3H, s, OMe), 6.69 (1H, s), 6.86 (2H, d, J=8.5 Hz), 7.48 (2H, d, J=8.5 Hz), 9.14 (1H, s), 5-ethynyl-7-methoxy-N-(4-(4-methylDiDerazin-1-yl)phenyl)vrido[2,3-d]pyrimidin-2-amine

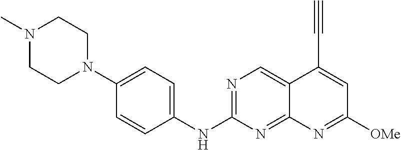 [SMILES: C#Cc1cc(OC)nc2nc(Nc3ccc(N4CCN(C)CC4)cc3)ncc12]

General Procedure 1 was applied to 7-methoxy-N-(4-(4-methylpiperazin-1-yl)phenyl)-5-((triisopropylsilyl)ethynyl)pyrido[2,3-d]pyrimidin-2-amine (90.0 mg, 0.170 mmol) with potassium fluoride (196 mg, 3.39 mmol) in DMF (2.5 mL). The crude material was purified by flash chromatography eluting with methanol (10%) in dichloromethane to yield the title compound as an orange solid.

(ES, m/z): [M+H]⁺=375.4;

¹H NMR (500 MHz, CDCl₃): b_{H}=2.38 (3H, s), 2.67 (4H, t, J=4.7 Hz), 3.19 (4H, t, J=4.7 Hz), 3.52 (1H, s), 4.04 (3H, s), 6.73 (1H, s), 6.89 (2H, d, J=9.0 Hz), 7.31 (1H, s), 7.49 (2H, d, J=9.0 Hz), 9.14 (1H, s).

### Example 4

N-[(3-nitrophenyl)methyl]cyclobutanamine

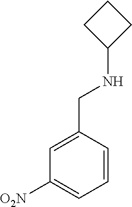 [SMILES: O=[N+]([O-])c1cccc(CNC2CCC2)c1]

General Procedure 4 was applied to 1-(bromomethyl)-3-nitrobenzene (4.00 g, 18.5 mmol), cyclobutylamine (1.97 g, 27.8 mmol) and triethylamine (4.68 g, 46.2 mmol) in dichloromethane (20 mL). The residue was purified by reverse phase flash column chromatography eluting with acetonitrile (15-20%) in water (formic acid, 0.4%) to yield the title compoundas a yellow liquid (1.45 g, 38%).

(ES, m/z): [M+H]r=207 N-methyl-N-[3-nitrophenyl)methyl]cyclobutanamine

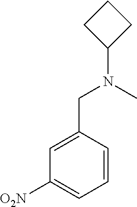 [SMILES: CN(Cc1cccc([N+](=O)[O-])c1)C1CCC1]

To a stirred solution of N-[(3-nitrophenyl)methyl]cyclobutanamine (1.45 g, 7.03 mmol, 1.0 eq.) in DMF (15 mL) was added sodium hydride (422 mg, 10.5 mmol, 1.5 eq.) at 0° C. under a nitrogen atmosphere. The reaction mixture was then stirred for 30 min at room temperature, followed by the addition of methyl iodide (1.50 g, 10.5 mmol, 1.5 eq.) in portions at 0° C. The resulting mixture was stirred for 2 h at room temperature under a nitrogen atmosphere. The resulting mixture was extracted with ethyl acetate (100 mL). The combined organic layers were washed with brine (5×25 mL), dried (Na₂SO₄) and concentrated under reduced pressure to afford the title compound as a yellow liquid (1.16 g, 75%).

(ES, m/z): [M+H]⁺=221

### 3-{[cyclobutyl(methyl)amino]methyl}aniline

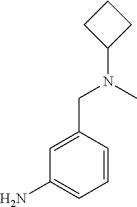

General Procedure 5 was applied to N-methyl-N-[(3-nitrophenyl)methyl]cyclobutanamine (1.16 g, 5.27 mmol) and palladium on carbon (232 mg, 2.18 mmol) in methanol (40 mL) was stirred for 3 h at room temperature under an hydrogen atmosphere. The resulting mixture was filtered, washing with methanol (3×2 mL). The filtrate was concentrated under reduced pressure to afford the title compound as a brown liquid (836 mg, 83%).

(ES, m/z): [M+H]⁺=191

### N-(3-{[cyclobutyl(methyl)amino]methyl}phenyl)-7-methoxy-5-[2-(triisopropylsilyl)ethynyl]pyrido[2,3-d]pyrimidin-2-amine

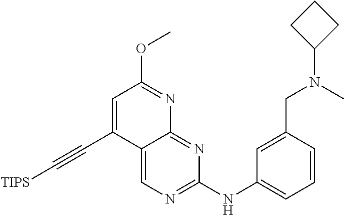

General Procedure 3 was applied to 2-methanesulfonyl-7-methoxy-5-[2-(triisopropylsilyl)ethynyl]pyrido[2,3-d]pyrimidine (500 mg, 1.19 mmol) 3-{[cyclobutyl(methyl)amino]methyl}aniline (340 mg, 1.79 mmol) and trifluoroacetic acid (272 mg, 2.38 mmol) in 2-butanol (3 mL). The residue was purified by reverse phase flash column chromatography eluting with acetonitrile (10-80%) in water (0.1% formic acid) to yield the title compound as a light yellow oil (230 mg, 36%).

(ES, m/z): [M+H]r=530

### N-(3-{[cyclobutyl(methyl)amino]methyl}phenyl)-5-ethynyl-7-methoxypyrido[2,3-d]pyrimidin-2-amine

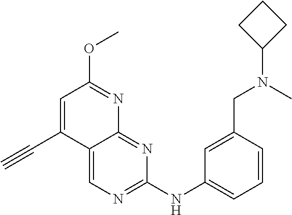 [SMILES: C#Cc1cc(OC)nc2nc(Nc3cccc(CN(C)C4CCC4)c3)ncc12]

General Procedure 1 deprotection was applied to N-(3-{[cyclobutyl(methyl)amino]methyl}phenyl)-7-methoxy-5-[2-(triisopropylsilyl)ethynyl]pyrido[2,3-d]pyrimidin-2-amine (200 mg, 0.377 mmol) and potassium fluoride (219 mg, 3.77 mmol) in THF (10 mL) and water (1 mL). The resulting mixture was stirred for 3 h at 60° C. under air atmosphere. The residue was purified by reverse phase flash column chromatography eluting with acetonitrile (10-100%) in water (0.1% formic acid) to yield the title compound as a light yellow solid (58 mg, 41%).

(ES, m/z): [M+H]⁺=374

¹H NMR (400 MHz, DMSO-d₆) δ 10.12 (s, 1H), 9.19 (s, 1H), 7.92-7.77 (m, 2H), 7.29 (t, J=7.8 Hz, 1H), 7.01-6.89 (m, 2H), 5.07 (s, 1H), 4.02 (s, 3H), 2.90 (p, J=7.7 Hz, 1H), 2.11-2.00 (m, 2H), 1.97 (s, 3H), 1.91-1.80 (m, 2H), 1.62 (ddd, J=20.3, 10.1, 7.4 Hz, 2H).

### Example 5

2-fluoro-N,N-dimethyl-5-nitroaniline

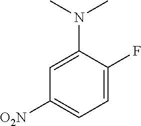

To a stirred mixture of 2-fluoro-5-nitroaniline (5.00 g, 32.0 mmol, 1.0 eq.) and formaldehyde (9.62 g, 320 mmol, 10 eq.) in methanol was added sodium cyanoborohydride (10.7 g, 170 mmol, 5.3 eq.) and acetic acid (37.5 mL) in portions at 0° C. then stirred for 1 h at room temperature. The resulting mixture was concentrated under reduced pressure. The residue was dissolved in water (50 mL) and extracted with dichloromethane (3×50 mL), dried (Na₂SO₄). The residue was purified by flash column chromatography eluting with ethyl acetate (10%) in 40-60 petroleum ether to afford the title compound as a dark red oil (4.00 g, 68%).

(ES, m/z): [M+H]-=185 N,N-dimethyl-2-(4-methylpiperazin-1-yi)-5-nitroaniline

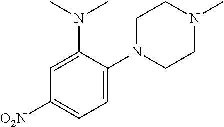 [SMILES: CN1CCN(c2ccc([N+](=O)[O-])cc2N(C)C)CC1]

General Procedure 6 was applied to 2-fluoro-N,N-dimethyl-5-nitroaniline (2.00 g, 10.9 mmol), 1-methylpiperazine (1.31 g, 13.0 mmol) and potassium carbonate (2.10 g, 15.2 mmol) in DMF. The residue was purified by flash column chromatography, eluting with dichloromethane (10%) methanol to afford the title compound as a brown yellow oil (2.40 g, 84%).

(ES, m/z): [M+H]⁺=265 N¹,N¹-dimethyl-6-(4-methylpiperazin-1-yl)benzene-1,3-diamine

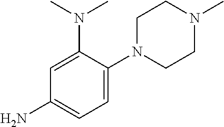 [SMILES: CN1CCN(c2ccc(N)cc2N(C)C)CC1]

General Procedure 5 was applied to N,N-dimethyl-2-(4-methylpiperazin-1-yl)-5-nitroaniline (1.20 g, 4.54 mmol) and palladium on carbon (3.00 g) in methanol (30 mL) to yield the title compound as a blue oil (905 mg, 85%).

(ES, m/z): [M+H]⁺=235

### N³-{7-methoxy-5-[2-(triisopropylsilyl)ethynyl]pyrido[2,3-d]pyrimidin-2-yl}—N¹,N¹-dimethyl-6-(4-methylpiperazin-1-yl)benzene-1,3-diamine

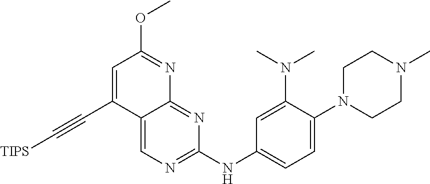

General Procedure 3 was applied to N¹,N¹-dimethyl-6-(4-methylpiperazin-1-yl)benzene-1,3-diamine (335 mg, 1.43 mmol), trifluoroacetic acid (217 mg, 1.91 mmol) and 2-methanesulfonyl-7-methoxy-5-[2-(triisopropylsilyl) ethynyl]pyrido[2,3-d]pyrimidine (400 mg, 0.953 mmol) in 2-methyl-2-butanol (5 mL) was stirred for 2 h at 100° C. The residue was purified by reverse phase flash column chromatography eluting with acetonitrile (10-50%) in water (0.4% formic acid) to yield the title compound as a red solid (234 mg, 43%).

(ES, m/z): [M+H]⁺=574

### N³-{5-ethynyl-7-methoxypyrido[2,3-d]pyrimidin-2-yl}-N¹,N¹-dimethyl-6-(4-methylpiperazin-1-yl)benzene-1,3-diamine

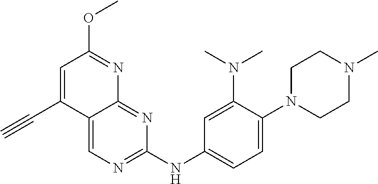 [SMILES: C#Cc1cc(OC)nc2nc(Nc3ccc(N4CCN(C)CC4)c(N(C)C)c3)ncc12]

General Procedure 1 was applied to N³-{7-methoxy-5-[2-(triisopropylsilyl)ethynyl]pyrido[2,3-d]pyrimidin-2-yl}-N¹,N1-dimethyl-6-(4-methylpiperazin-1-yl)benzene-1,3-diamine (30.0 mg, 52.0 μmol) and potassium fluoride (30.4 mg, 0.520 mmol) in THF (2 mL), water (0.1 mL) and DMF (2 mL) was stirred for 1 h at room temperature. The residue was purified by reverse phase flash column chromatography eluting with acetonitrile (10-50%) in water (0.4% formic acid) to yield the title compound as a red solid (41 mg, 26%).

(ES, m/z): [M+H]⁺=418

¹H NMR (300 MHz, DMSO-d₆) δ 9.88 (s, 1H), 9.14 (s, 1H), 7.54 (d, J=8.4 Hz, 1H), 7.38 (d, J=2.4 Hz, 1H), 6.94 (s, 1H), 6.87 (d, J=8.7 Hz, 1H), 5.05 (s, 1H), 4.25 (s, 4H), 4.01 (s, 4H), 3.02 (s, 4H), 2.81 (s, 6H), 2.55 (s, 4H), 2.25 (s, 3H).

### Example 6

2-fluoro-5-nitro-N-phenyl)aniline

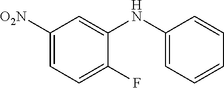 [SMILES: O=[N+]([O-])c1ccc(F)c(Nc2ccccc2)c1]

A mixture of 2-bromo-1-fluoro-4-nitrobenzene (500 mg, 2.27 mmol, 1 eq.), XantPhos (263 mg, 0.455 mmol, 0.2 eq.), cesium carbonate (1.48 g, 4.55 mmol, 2.0 eq.) and tris(dibenzyldeneacetone)dipalladium(0) (208 mg, 0.227 mmol, 0.1 eq.) in 1,4-dioxane was stirred for 2 h at 100° C. The mixture was cooled to room temperature then diluted with water (50 mL) and extracted with ethyl acetate (3×30 mL). The combined organic layers were dried (Na₂SO₄) and concentrated under reduced pressure to yield the title compound as a yellow solid (517 mg, 98%).

(ES, m/z): [M+H]⁺=233

### 2-(4-methylpiperazin-1-yl)-5-nitro-N-phenyl)aniline

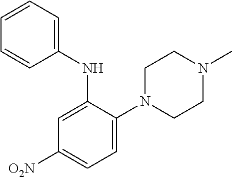 [SMILES: CN1CCN(c2ccc([N+](=O)[O-])cc2Nc2ccccc2)CC1]

General Procedure 6 was applied to 2-fluoro-5-nitro-N-phenyl)aniline (517 mg, 2.23 mmol), 1-methylpiperidine and potassium carbonate (738 mg, 5.34 mmol) in DMF was stirred for 1 h at 80° C. The residue was purified by flash column chromatography, eluting with ethyl acetate (10%) in 40-60 petroleum ether to yield the title compound as a brown yellow oil (500 mg, 72%).

(ES, m/z): [M+H]⁺=313

### 6-(4-methylpiperazin-1-yl)-N1-phenylbenzene-1,3-diamine

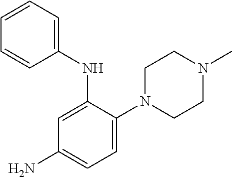 [SMILES: CN1CCN(c2ccc(N)cc2Nc2ccccc2)CC1]

General Procedure 5 was applied to 2-(4-methylpiperazin-1-yl)-5-nitro-N-phenylaniline (500 mg, 1.60 mmol) and palladium on carbon (10%, 0.85 g) in methanol (15 mL) to yield the title compound as a reddish oil (350 mg, 77%).

(ES, m/z): [M+H]⁺=283

### N¹-{7-methoxy-5-[2-(triisopropylsilyl)ethynyl]pyrido[2,3-d]pyrimidin-2-yl}-4-(4-methylpiperazin-1-yl)-N³-phenylbenzene-1,3-diamine

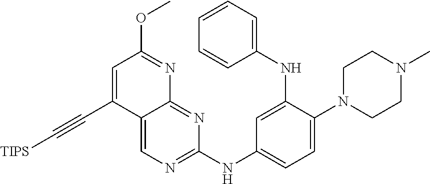

General Procedure 3 was applied to 6-(4-methylpiperazin-1-yl)-N¹-phenylbenzene-1,3-diamine (300 mg, 1.06 mmol), 2-methanesulfonyl-7-methoxy-5-[2-(triisopropylsilyl)ethynyl]pyrido[2,3-d]pyrimidine (297 mg, 0.708 mmol) and trifluoroacetic acid (162 mg, 1.42 mmol) in 2-butanol (5 mL). The resulting mixture was stirred for 2 h at 100° C.

The residue was purified by reverse phase flash column chromatography eluting with acetonitrile (10-50%) in water (0.4% formic acid) to yield the title compound as a light yellow solid (110 mg, 25%).

(ES, m/z): [M+H]⁺=622

### N¹-{5-ethynyl-7-methoxypyrido[2,3-d]pyrimidin-2-yl}-4-(4-methylpiperazin -1-yl)-N³-phenylbenzene-1,3-diamine

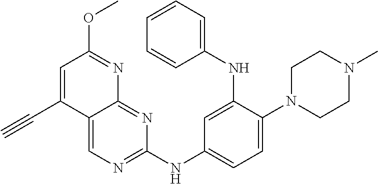 [SMILES: C#Cc1cc(OC)nc2nc(Nc3ccc(N4CCN(C)CC4)c(Nc4ccccc4)c3)ncc12]

General Procedure 1 was applied to N¹-{7-methoxy-5-[2-(triisopropylsilyl)ethynyl]pyrido[2,3-d]pyrimidin-2-yl}-4-(4-methylpiperazin-1-yl)-N³-phenylbenzene-1,3-diamine (90.0 mg, 0.145 mmol) and potassium fluoride (84.1 mg, 1.45 mmol) in THF (3 mL), water (0.5 mL) and DMF (1 mL). The reaction was stirred for 1 h at room temperature. The residue was purified by reverse phase flash column chromatography eluting with acetonitrile (10-50%) in water (0.4% formic acid) to afford the title compound as a red solid (25.1 mg, 37%).

(ES, m/z): [M+H]⁺=466

¹H NMR (300 MHz, DMSO-d₆) δ 9.98 (s, 1H), 9.15 (s, 1H), 7.93 (d, J=2.4 Hz, 1H), 7.36 (dd, J=8.6, 2.4 Hz, 1H), 7.25 (d, J=5.2 Hz, 4H), 7.16-7.03 (m, 2H), 6.96 (s, 1H), 6.89-6.77 (m, 1H), 5.06 (s, 1H), 4.01 (s, 3H), 2.84 (t, J=4.7 Hz, 4H), 2.24 (s, 3H).

### Example 7

### N-methyl-N-[(3-nitrophenyl)methyl]acetamide

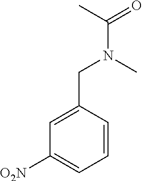

A solution of 3-nitrobenzaldehyde (10.0 g, 66.72 mmol, 1.0 eq.), methylamine (2M in methanol, 0.331 mL, 662 mmol, 10 eq.), sodium cyanoborohydride (4.16 g, 66.2 mmol, 1.0 eq.) was stirred for 16 hr at room temperature. The solution was cooled to 0° C., and acetic anhydride (67.6 g, 661.7 mmol, 10 eq.) was added, then stirred for 30 minutes at room temperature. The solution was diluted with ethyl acetate (25 mL) and washed with aqueous 10% citric acid solution (25 mL), saturated aqueous sodium bicarbonate solution (25 mL), and brine (25 mL). The organic layer was dried (MgSO₄) and concentrated under reduced pressure to yield the title compound as an orange oil (9.00 g, 65%).

(ES, m/z): [M+H]⁺=209

### N-[(3-aminophenyl)methyl]-N-methylacetamide

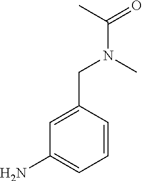

General Procedure 5 was applied to N-methyl-N-[(3-nitrophenyl)methyl]acetamide (1.00 g, 4.80 mmol), with palladium on carbon (200 mg, 1.88 mmol) in methanol (10 mL) to yield the title compound as an orange oil (800 mg, 93%).

(ES, m/z): [M+H]⁺=179

### N-{[3-({7-methoxy-5-[2-(triisopropylsilyl)ethynyl]pyrido[2,3-d]pyrimidin-2-yl}amino)phenyl]methyl}-N-methylacetamide

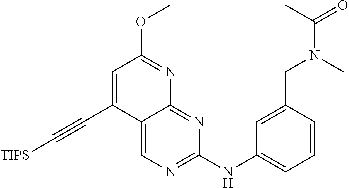

General Procedure 3 was applied to 2-methanesulfonyl-7-methoxy-5-[2-(triisopropylsilyl)ethynyl]pyrido[2,3-d]pyrimidine (500 mg, 1.19 mmol), N-[(3-aminophenyl)methyl]-N-methylacetamide (318 mg, 1.79 mmol) and trifluoroacetic acid (0.18 mL, 2.38 mmol) in 2-butanol (5 mL). The resulting mixture was stirred for 2 h at 60° C. The residue was purified by reverse phase flash column chromatography eluting with acetonitrile (50-90%) in water (0.4% formic acid) to yield the title compound as a yellow solid (262 mg, 42%).

(ES, m/z): [M+H]⁺=518

### N-{[3-({5-ethynyl-7-methoxypyrido[2,3-d]pyrimidin-2-yl}amino)pheny]methyl}-N-methylacetamide

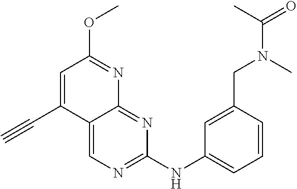 [SMILES: C#Cc1cc(OC)nc2nc(Nc3cccc(CN(C)C(C)=O)c3)ncc12]

General Procedure 1 was applied to N-{[3-({7-methoxy-5-[2-(triisopropylsilyl)ethynyl]pyrido[2,3-d]pyrimidin-2-yl}amino)phenyl]methyl}-N-methylacetamide (253 mg, 0.489 mmol) and potassium fluoride (284 mg, 4.89 mmol) in THF (5 mL) and water (1 mL). The resulting mixture was stirred for 3 h at 60° C. The residue was purified by reverse phase flash column chromatography eluting with acetonitrile (10-80%) in water (0.1% formic acid) to yield the title compound as a light yellow solid (115 mg, 65%).

(ES, m/z): [M+H]⁺=362

¹H NMR (300 MHz, DMSO-d₆) δ 10.19 (d, J=23.7 Hz, 1H), 9.19 (s, 1H), 8.00-7.68 (m, 2H), 7.33 (dt, J=11.1, 7.8 Hz, 1H), 7.00 (d, J=2.1 Hz, 1H), 6.89 (t, J=6.1 Hz, 1H), 5.08 (s, 1H), 4.54 (d, J=13.9 Hz, 2H), 4.03 (d, J=2.8 Hz, 3H), 2.94 (d, J=48.0 Hz, 3H), 2.13 (d, J=5.4 Hz, 3H).

### Example 8

### N-cyclobutyl-2-fluoro-5-nitroaniline

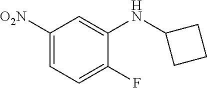 [SMILES: O=[N+]([O-])c1ccc(F)c(NC2CCC2)c1]

A solution of 2-fluoro-5-nitroaniline (1.00 g, 6.41 mmol, 1.0 eq.), cyclobutanone (673 mg, 9.61 mmol, 1.5 eq.) and acetic acid (76.9 mg, 1.28 mmol, 0.2 eq.) in dichloromethane (10 mL) was stirred for 2 h at room temperature then sodium triacetoxyborohydride (2.72 g, 12.8 mmol, 2.0 eq.) was added in portions before stirring overnight at room temperature. The resulting mixture was extracted with ethyl acetate (3×50 mL). The combined organic layers were washed with saturated aqueous sodium hydrogen carbonate solution (2×50 mL) and brine (2×50 mL), dried (Na₂SO₄) and concentrated under reduced pressure. The residue was purified by flash column chromatography, eluting with ethyl acetate (0-5%) in 40-60 petroleum ether to afford the title compound as a yellow solid (275 mg, 20%).

(ES, m/z): [M+H]⁺=211

### N-cyclobutyl-2-(4-methylpiperazin-1-yl)-5-nitroaniline

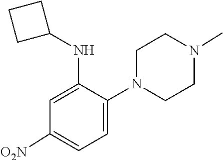 [SMILES: CN1CCN(c2ccc([N+](=O)[O-])cc2NC2CCC2)CC1]

General Procedure 6 was applied to N-cyclobutyl-2-fluoro-5-nitroaniline (255 mg, 1.21 mmol), 1-methylpiperazine (146 mg, 1.46 mmol) and potassium carbonate (570 mg, 4.12 mmol) in DMF (3 mL). The residue was purified by flash column chromatography eluting with ethyl acetate (65%) in 40-60 petroleum ether to afford the title compound as a yellow solid (181 mg, 51%).

(ES, m/z): [M+H]⁺=291

### N¹-cyclobutyl-6-(4-methylpiperazin-1-yl)benzene -1,3-diamine

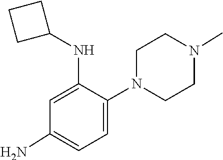 [SMILES: CN1CCN(c2ccc(N)cc2NC2CCC2)CC1]

General Procedure 5 was applied to N-cyclobutyl-2-(4-methylpiperazin-1-yl)-5-nitroaniline (1.66 g, 5.71 mmol) and palladium on carbon (182 mg, 1.71 mmol) in methanol (10 mL) to yield the title compound as a black solid (1.38 g, 93%).

(ES, m/z): [M+H]⁺=261

### N³-cyclobutyl-N¹-{7-methoxy-5-[2-(triisopropylsilyl)ethynyl]pyrido[2,3-d]pyrimidin-2-y}-4-(4-methylpiperazin-1-yl)benzene-1.3-diamine

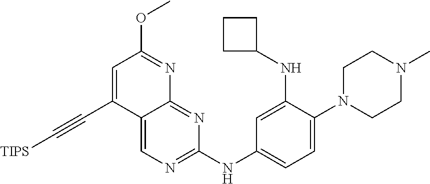

General Procedure 3 was applied to 2-methanesulfonyl-7-methoxy-5-[2-(triisopropylsilyl)ethynyl]pyrido[2,3-d]pyrimidine (400 mg, 0.953 mmol), N¹-cyclobutyl-6-(4-methylpiperazin-1-yl)benzene-1,3-diamine (372 mg, 1.43 mmol) and trifluoroacetic acid (0.140 mL, 1.91 mmol) in 2-butanol (5 mL). The resulting mixture was stirred for 2 h at 60° C. The residue was purified by reverse phase flash column chromatography eluting with methanol (50-80%) in water (0.1% formic acid) to yield the title compound as a red solid (297 mg, 52%).

(ES, m/z): [M+H]⁺=600

### N³-cyclobutyl-N¹-{5-ethynyl-7-methoxypyrido[2,3-d]pyrimidin-2-yl}-4-(4-methylpiperazin-1-yl)benzene-1,3-diamine

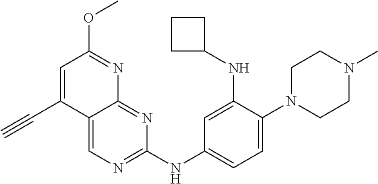 [SMILES: C#Cc1cc(OC)nc2nc(Nc3ccc(N4CCN(C)CC4)c(NC4CCC4)c3)ncc12]

General Procedure 1 was applied to N³-cyclobutyl-N¹-{7-methoxy-5-[2-(triisopropylsilyl)ethynyl]pyrido[2,3-d]pyrimidin-2-yl}-4-(4-methylpiperazin-1-yl)benzene-1,3-diamine (290 mg, 0.483 mmol) and potassium fluoride (281 mg, 4.83 mmol) in THE (5 mL) and water (1 mL). The resulting mixture was stirred for 3 h at 60° C. The residue was purified by reverse phase flash column chromatography eluting with acetonitrile (10-50%) in water (0.1% formic acid) to yield the title compound as a yellow solid (69.5 mg, 32%).

(ES, m/z): [M+H]⁺=600

¹H NMR (300 MHz, DMSO-d₆) δ 9.89 (s, 1H), 9.14 (s, 1H), 7.46 (s, 1H), 7.04 (dd, J=8.5, 2.3 Hz, 1H), 6.98-6.84 (m, 2H), 5.06 (s, 1H), 4.81 (d, J=6.1 Hz, 1H), 4.00 (s, 3H), 3.85 (q, J=7.2 Hz, 2H), 2.78 (t, J=4.7 Hz, 4H), 2.25 (s, 3H), 1.92-1.67 (m, 4H), 1.24 (s, 1H).

### Example 9

### N-{3-[(dimethylamino)methyl]phenyl}-7-methoxy-5-[2-(triisopropylsilyl)ethynyl]pyrido[2,3-d]pyrimidin-2-amine

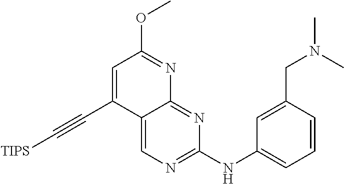

General Procedure 3 was applied to 2-methanesulfonyl-7-methoxy-5-[2-(triisopropylsilyl)ethynyl]pyrido[2,3-d]pyrimidine (450 mg, 1.07 mmol), 3-[(methylamino)methyl]aniline (219 mg, 1.61 mmol) and trifluoroacetic acid (0.16 mL, 2.14 mmol) in 2-butanol (5 mL). The resulting mixture was stirred for 4 h at 60° C. The residue was purified by reverse phase flash column chromatography eluting with acetonitrile (10-100%) in water (0.1% formic acid) to yield the title compound as a light yellow oil (221 mg, 42%).

(ES, m/z): [M+H]⁺=490

### N-{3-[(dimethylamino)methyl]phenyl}-5-ethynyl-7-methoxypyrido[2,3-d] pyrimidin-2-amine

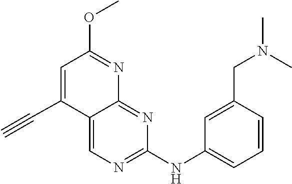 [SMILES: C#Cc1cc(OC)nc2nc(Nc3cccc(CN(C)C)c3)ncc12]

General Procedure 1 was applied to N-{3-[(dimethylamino)methyl]phenyl}-7-methoxy-5-[2-(triisopropylsilyl)ethynyl]pyrido[2,3-d]pyrimidin-2-amine (170 mg, 0.347 mmol) and potassium fluoride (202 mg, 3.47 mmol) in THF (5 mL) and water (0.5 mL). The resulting mixture was stirred for 4 h at 70° C. The residue was purified by reverse phase flash column chromatography eluting with acetonitrile (50-100%) in water (0.01% formic acid) to afford the title compound as a light yellow solid (19.0 mg, 16%).

(ES, m/z): [M+H]⁺=334

¹H NMR (400 MHz, DMSO-d₆) δ 10.16 (s, 1H), 9.20 (s, 1H), 7.96 (d, J=8.3 Hz, 1H), 7.72 (t, J=1.9 Hz, 1H), 7.34 (t, J=7.8 Hz, 1H), 7.02 (d, J=9.2 Hz, 2H), 5.08 (s, 1H), 4.02 (s, 3H), 3.59 (s, 2H), 2.31 (s, 6H).

### Example 10

### N-(cyclobutylmethyl)-2-fluoro-5-nitroaniline

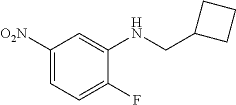 [SMILES: O=[N+]([O-])c1ccc(F)c(NCC2CCC2)c1]

A mixture of 2-fluoro-5-nitroaniline (4.00 g, 25.6 mmol, 1.0 eq.), cyclobutyral (3.23 g, 38.4 mmol, 1.5 eq.) and acetic acid (308 mg, 5.12 mmol, 0.2 eq.) in 1,2-dichloroethane (40 mL) was stirred for 1 h at room temperature. Sodium triacetoxyborohydride (10.8 g, 51.2 mmol, 2.0 eq.) was added in portions over 5 minutes. The resulting mixture was stirred for an additional 3 h at room temperature then diluted with water (30 mL). The aqueous layer was extracted with dichloromethane (2×50 mL). The residue was purified by reverse phase flash column chromatography eluting with acetonitrile (70-80%) in water (0.4% formic acid) to yield the title compound as a yellow oil (1.76 g, 31%).

(ES, m/z): [M+H]⁺=225.1

### N-(cyclobutylmethyl)-2-(4-methylpiperazin-1-yl)-5-nitroaniline

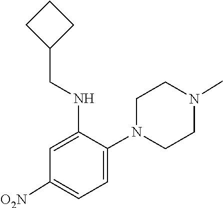 [SMILES: CN1CCN(c2ccc([N+](=O)[O-])cc2NCC2CCC2)CC1]

General Procedure 6 was applied to N-(cyclobutylmethyl)-2-fluoro-5-nitroaniline (1.76 g, 7.85 mmol), 1-methylpiperazine (1.18 g, 11.8 mmol) and potassium carbonate (2.71 g, 19.6 mmol) in DMF (15 mL). The reaction mixture was stirred for 12 h at 100° C. The residue was purified by flash column chromatography eluting with ethyl acetate (50%) in 40-60 petroleum ether to afford the title compound as an orange solid (1.63 g, 68%).

(ES, m/z): [M+H]⁺=305.2

### N¹-(cyclobutylmethyl)-6-(4-methylpiperazin-1-yl)benzene-1,3-diamine

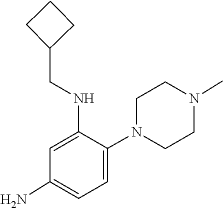 [SMILES: CN1CCN(c2ccc(N)cc2NCC2CCC2)CC1]

General Procedure 5 was applied to N-(cyclobutylmethyl)-2-(4-methylpiperazin-1-yl)-5-nitroaniline (800 mg, 2.63 mmol, 1 eq.) and palladium on carbon (160 mg, 1.50 mmol) in methanol (40 mL) to afford the title compound as a brown oil (679 mg, 94%).

(ES, m/z): [M+H]⁺=275.2

### N³-(cyclobutylmethyl)-N¹-{7-methoxy-5-[2-(triisopropylsilyl)ethynyl]pyrido[2,3-d]pyrimidin-2-yl}-4-(4-methylpiperazin-1-y)benzene-1,3-diamine

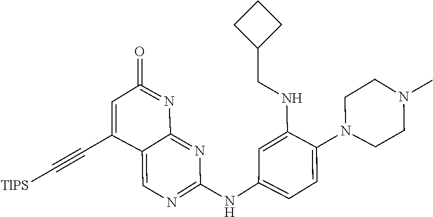

General Procedure 3 was applied to 2-methanesulfonyl-7-methoxy-5-[2-(triisopropylsilyl)ethynyl]pyrido[2,3-d]pyrimidine (592 mg, 1.41 mmol), N¹-(cyclobutylmethyl)-6-(4-methylpiperazin-1-yl)benzene-1,3-diamine (581 mg, 2.12 mmol) and trifluoroacetic acid (322 mg, 2.82 mmol) in 2-butanol (4 mL). The reaction was stirred overnight at 60° C. The residue was purified by reverse phase flash column chromatography eluting with acetonitrile in water (0.1% formic acid) to afford the title compound as an orange solid (201 mg, 23%).

(ES, m/z): [M+H]⁺=614.4; 328.3

### N³-(cyclobutylmethyl)-N¹-{5-ethynyl-7-methoxypyrido[2,3-d]pyrimidin-2-yl}-4-(4-methylpiperazin-1-yl)benzene-1,3-diamine

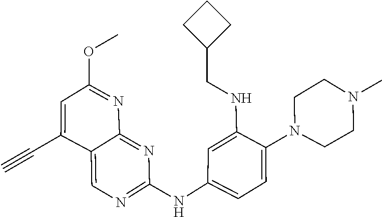 [SMILES: C#Cc1cc(OC)nc2nc(Nc3ccc(N4CCN(C)CC4)c(NCC4CCC4)c3)ncc12]

General Procedure 1 was applied to N³-(cyclobutylmethyl)-N¹-{7-methoxy-5-[2-(triisopropylsilyl)ethynyl]pyrido[2,3-d]pyrimidin-2-yl}-4-(4-methylpiperazin-1-yl)benzene-1,3-diamine (20.0 mg, 33.0 μmol) and potassium fluoride (170 mg, 2.93 mmol) in DMF (1 mL), THE (1 mL) and water (100 uL) was stirred for 2 h at 60° C. The residue was purified by reverse phase flash column chromatography eluting with acetonitrile (20-30%) in water (0.1% formic acid) to afford the title compound as a yellow solid (8.90 mg, 6%).

(ES, m/z): [M+H]⁺=458.2

¹H NMR (400 MHz, DMSO-d₆) δ 9.85 (s, 1H), 9.14 (s, 1H), 7.24 (s, 1H), 7.14 (d, J=8.4 Hz, 1H), 6.94 (d, J=9.3 Hz, 2H), 5.05 (s, 1H), 4.85 (s, 1H), 4.01 (s, 3H), 3.13 (t, J=6.5 Hz, 2H), 2.84 (s, 8H), 2.67 (d, J=7.2 Hz, 1H), 2.47 (s, 3H), 2.10-2.03 (m, 2H), 1.89 (dd, J=10.3, 5.9 Hz, 2H), 1.80-1.72 (m, 2H).

### Example 11

### 2-(4-methylpiperazin-1-yl)-5-nitroaniline

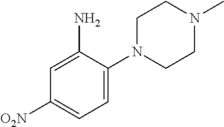 [SMILES: CN1CCN(c2ccc([N+](=O)[O-])cc2N)CC1]

A mixture of 2-fluoro-5-nitroaniline (5.00 g, 32.0 mmol, 1.0 eq.) and 1-methylpiperazine (9.62 g, 96.1 mmol, 3.0 eq.) in N,N-diisopropyl-N-ethylamine (6.21 g, 48.0 mmol, 1.5 eq.) was stirred for 3 h at 80° C. The mixture was cooled to 0° C. and the precipitated solids collected by filtration, washing with dichloromethane (10 mL) to yield the title compound as a yellow solid (3.10 g, 41%).

(ES, m/z): [M+H]⁺=237

### 1-methyl-N-[2-(4-methylpiperazin-1-yl)-5-nitrophenyl]pyrazole-3-carboxamide

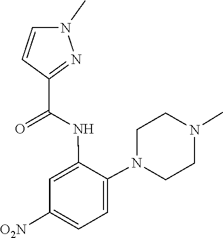 [SMILES: CN1CCN(c2ccc([N+](=O)[O-])cc2NC(=O)c2ccn(C)n2)CC1]

A mixture of 2-(4-methylpiperazin-1-yl)-5-nitroaniline (3.00 g, 12.7 mmol, 1.0 eq.) and 1-methylpyrazole-3-carboxylic acid (4.80 g, 38.1 mmol, 3.0 eq.) and N,N-diisopropyl-N-ethylamine (4.42 mL, 25.4 mmol, 2.0 eq.) in dichloromethane (30 mL) was stirred for 1.5 h at room temperature. The resulting mixture was diluted with water (100 mL) and extracted with dichloromethane (2×100 mL). The combined organic layers were dried (Na₂SO₄) and concentrated under reduced pressure to yield the title compound as a yellow solid (1.50 g, 34%).

(ES, m/z): [M+H]⁺=345

### 2-(4-methylpiperazin-1-yl)-N-[(1-methylpyrazol-3-yl)methyl]-5-nitroaniline

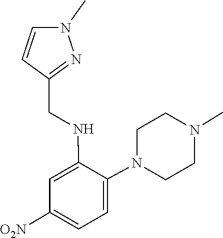 [SMILES: CN1CCN(c2ccc([N+](=O)[O-])cc2NCc2ccn(C)n2)CC1]

A mixture of 1-methyl-N-[2-(4-methylpiperazin-1-yl)-5-nitrophenyl]pyrazole-3-carboxamide (1.50 g, 4.36 mmol, 1.0 eq.) and borane-tetrahydrofuran (0.83 mL, 8.71 mmol, 2.0 eq.) in THF (20 mL) was stirred overnight at 80° C. The mixture was allowed to cool to room temperature and 2M aqueous sodium hydroxide solution (21.8 mL, 43.6 mmol, 10 eq.) was added dropwise over 5 minutes. The resulting mixture was stirred for additional 2 h at 80° C. then cooled to room temperature and extracted with ethyl acetate (3×50 mL). The combined organic layers were dried (Na₂SO₄) and concentrated under reduced pressure to yield the title compound as a reddish solid (1.30 g, 90%).

(ES, m/z): [M+H]⁺=331

### 6-(4-methylpiperazin-1-yl)-N¹-[(1-methylpyrazol-3-yl)methyl]benzene-1,3-diamine

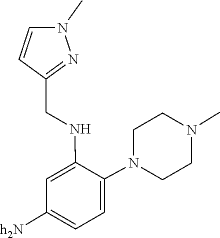

General Procedure 5 was applied to 2-(4-methylpiperazin-1-yl)-N-[(1-methylpyrazol-3-yl)methyl]-5-nitroaniline (1.30 g, 3.94 mmol) and palladium on carbon (2.09 g) in methanol (20 mL) to yield the title compound as a black oil (861 mg, 73%).

(ES, m/z): [M+H]⁺=301

### N¹-{7-methoxy-5-[2-(triisopropylsilyl)ethynyl]pyrido[2,3-d]pyrimidin-2-yl}-4-(4-methylpiperazin-1-yl)-N³-[(1-methylpyrazol-3-yl)methyl]benzene-1,3-diamine

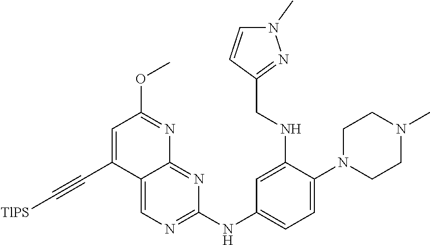

General Procedure 3 was applied to 2-methanesulfonyl-7-methoxy-5-[2-(triisopropylsilyl)ethynyl]pyrido[2,3-d]pyrimidine (500 mg, 1.19 mmol), 6-(4-methylpiperazin-1-yl)-N¹-[(1-methylpyrazol-3-yl)methyl]benzene-1, 3-diamine (537 mg, 1.79 mmol) and trifluoroacetic acid (272 mg, 2.38 mmol) in 2-butanol (5 mL). The reaction was stirred for 3 h at 80° C. The residue was purified by reverse phase flash column chromatography eluting with acetonitrile (10-50%) in water (0.1% formic acid) to yield the title compound as a light yellow solid (284 mg, 37%).

(ES, m/z): [M+H]⁺=640

### N¹-{5-ethynyl-7-methoxypyrido[2,3-d]pyrimidin-2-yl}-4-(4-methylpiperazin-1-yl)-N³-[(1-methylpyrazol-3-yl)methyl]benzene-1,3-diamine

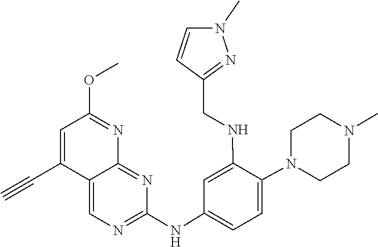 [SMILES: C#Cc1cc(OC)nc2nc(Nc3ccc(N4CCN(C)CC4)c(NCc4ccn(C)n4)c3)ncc12]

General Procedure 1 was applied to N¹-{7-methoxy-5-[2-(triisopropylsilyl)ethynyl]pyrido[2,3-d]pyrimidin-2-yl}-4-(4-methylpiperazin-1-yl)-N³-[(1-methylpyrazol-3-yl)methyl]benzene-1,3-diamine (234 mg, 0.366 mmol) and potassium fluoride (212 mg, 3.66 mmol) in THE (5 mL) and water (0.8 mL). The reaction was stirred for 1.5 h at 60° C. The residue was purified by reverse phase flash column chromatography eluting with acetonitrile (10-70%) in water (0.1% formic acid) to yield the title compound as a light yellow solid (9.00 mg, 5%).

(ES, m/z): [M+H]⁺=484

¹H NMR (300 MHz, DMSO-d₆) δ 9.85 (s, 1H), 9.14 (s, 1H), 8.29 (s, 1H), 7.60 (d, J=2.2 Hz, 1H), 7.21 (s, 2H), 6.96 (d, J=6.5 Hz, 2H), 6.31 (d, J=2.4 Hz, 1H), 5.27 (s, 1H), 5.06 (s, 1H), 4.23 (d, J=4.8 Hz, 2H), 4.00 (s, 2H), 3.80 (s, 3H), 2.80 (s, 4H), 2.25 (s, 3H).

### Example 12

### 2-(methylsulfanyl)-7-phenoxy-5-[2-(triisopropylsilyl)ethynyl]pyrido[2,3-d]pyrimidine

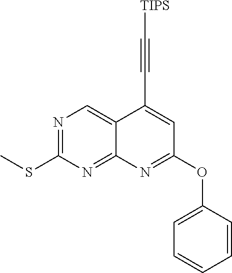

A solution of phenol (144 mg, 1.53 mmol, 1.2 eq.) and cesium carbonate (623 mg, 1.91 mmol, 1.5 eq.) in 1,4-dioxane (12 mL) was stirred for 20 min at room temperature then 7-chloro-2-(methylsulfanyl)-5-[2-(triisopropylsilyl)ethynyl]pyrido[2,3-d]pyrimidine (500 mg, 1.28 mmol, 1.0 eq.) was added. The resulting mixture was stirred for additional 4 h at room temperature then diluted with water (40 mL) and extracted with ethyl acetate (3×50 mL). The combined organic layers were washed with brine (40 mL), dried (Na₂SO₄) and concentrated under reduced pressure. The residue was purified by flash column chromatography eluting with ethyl acetate (6%) in 40-60 petroleum ether to afford the title compound as a light yellow solid (366 mg, 64%).

(ES, m/z): [M+H]⁺=450.2 2-methanesulfonyl-7-phenoxy-5-[2-(triisopropylsilyl)ethyn-yl]pyrido[2,3-d]pyrimidine

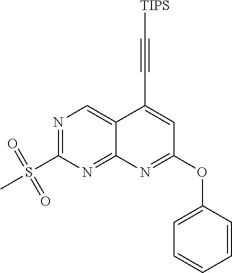

General Procedure 2 was applied to 2-(methylsulfanyl)-7-phenoxy-5-[2-(triisopropylsilyl)ethynyl]pyrido[2,3-d]pyrimidine (300 mg, 0.667 mmol) m-CPBA (253 mg, 1.47 mmol) in dichloromethane (12 mL) to afford the title compound as a yellow solid (310 mg, 96%), which was used in the next step without further purification.

(ES, m/z): [M+H]⁺=482.2

### N-[4-(4-methylpiperazin-1-yl)phenyl]-7-phenoxy-5-[2-(triisopropylsilyl)ethynyl]pyrido[2,3-d]pyrimidin-2-amine

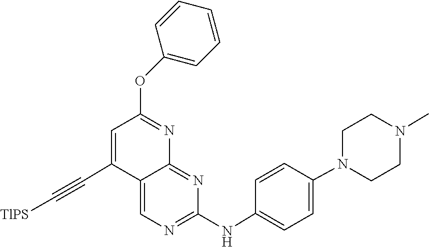

General Procedure 3 was applied to 2-methanesulfonyl-7-phenoxy-5-[2-(triisopropylsilyl)ethynyl]pyrido[2,3-d]pyrimidine (238 mg, 0.494 mmol), 4-(4-methylpiperazin-1-yl)aniline (142 mg, 0.741 mmol) and trifluoroacetic acid (113 mg, 0.988 mmol) in 2-butanol (10 mL). The reaction was stirred for 2 h at 50° C. The residue was purified by reverse phase flash column chromatography eluting with acetonitrile (40-65%) in water (0.4% formic acid) to afford the title compound as an orange solid (130 mg, 44%).

(ES, m/z): [M+H]⁺=593.3

### 5-ethynyl-N-[4-(4-methylpiperazin-1-yl)phenyl]-7-phenoxypyrido[2,3-d]pyrimidin-2-amine

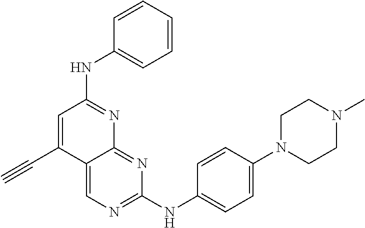 [SMILES: C#Cc1cc(Nc2ccccc2)nc2nc(Nc3ccc(N4CCN(C)CC4)cc3)ncc12]

General Procedure 1 was applied to N-[4-(4-methylpiperazin-1-yl)phenyl]-7-phenoxy-5-[2-(triisopropylsilyl) ethynyl]pyrido[2,3-d]pyrimidin-2-amine (100 mg, 0.169 mmol) and potassium fluoride (98.0 mg, 1.69 mmol) in DMF (2 mL), THF (2 mL) and water (200 uL). The resulting mixture was stirred for 1 h at 60° C. The residue was purified by reverse phase flash column chromatography eluting with acetonitrile (30-50%) in water (0.4% formic acid) in water to afford the title compound as a yellow solid (10.0 mg, 13%).

(ES, m/z): [M+H]⁺=437.1

¹H NMR (400 MHz, DMSO-d₆) δ 9.94 (s, 1H), 9.19 (s, 1H), 7.59 (d, J=8.3 Hz, 1H), 7.49 (t, J=7.8 Hz, 2H), 7.38 (d, J=5.2 Hz, 1H), 7.29 (dd, J=20.4, 7.6 Hz, 3H), 7.15 (s, 1H), 6.89 (d, J=8.7 Hz, 2H), 5.14 (s, 1H), 3.06 (t, J=5.0 Hz, 4H), 2.43 (t, J=4.9 Hz, 4H), 2.21 (s, 3H).

### Example 13

4-({[2-(methylsulfanyl)-5-[2-(triisopropylsilyl)ethynyl]pyrido[2,3-d]pyrimidin-7-yl]oxy}methyl)-1,3-thiazole

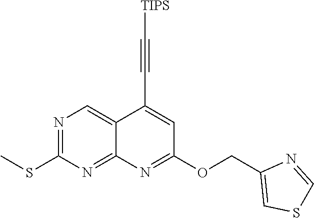

A mixture of 1,3-thiazol-4-ylmethanol (212 mg, 1.84 mmol, 1.2 eq.) and potassium tert-butoxide (258 mg, 2.29 mmol, 1.5 eq.) in 1,4-dioxane (15 mL) was stirred for 20 minutes at room temperature then 7-chloro-2-(methylsulfanyl)-5-[2-(triisopropylsilyl)ethynyl]pyrido[2,3-d]pyrimidine (600 mg, 1.53 mmol, 1.0 eq.) was added. The resulting mixture was stirred for 4 h at room temperature then diluted with water (15 mL) and extracted with ethyl acetate (3×20 mL). The combined organic layers were washed with brine (20 mL), dried (Na₂SO₄) and concentrated under reduced pressure. The residue was purified by silica gel column chromatography eluting with ethyl acetate (18%) in 40-60 petroleum ether to afford the title compound as a light yellow solid (143 mg, 20%).

(ES, m/z): [M+H]⁺=471.2

### 4-[({2-methanesulfonyl-5-[2-(triisopropylsilyl)ethynyl]pyrido[2,3-d]pyrimidin-7-yl}oxy)methyl]-1,3-thiazole

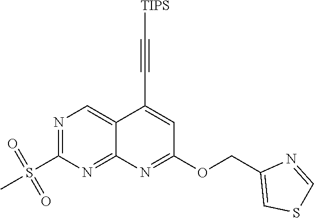

General Procedure 2 was applied to 4-({[2-(methylsulfanyl)-5-[2-(triisopropylsilyl)ethynyl]pyrido[2,3-d]pyrimidin-7-yl]oxy}methyl)-1,3-thiazole (310 mg, 0.659 mmol) and m-CPBA (250 mg, 1.45 mmol) in dichloromethane (12 mL) to afford the title compound as a yellow solid (325 mg, 98%), which was used in the next step without further purification.

(ES, m/z): [M+H]⁺=503.2

### N-[4-(4-methylpiperazin-1-yl)phenyl]-7-(1,3-thiazol-4-ylmethoxy)-5-[2-(triisopropylsilyl)ethynyl]pyrido[2,3-d]pyrimidin-2-amine

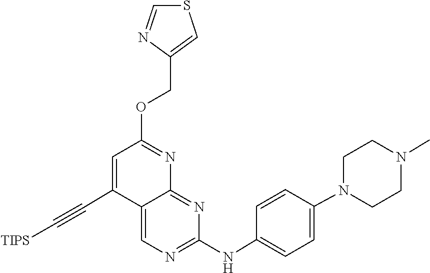

General Procedure 3 was applied to 4-[({2-methanesulfonyl-5-[2-(triisopropylsilyl)ethynyl]pyrido[2,3-d]pyrimidin-7-yl}oxy) methyl]-1,3-thiazole (325 mg, 0.646 mmol), 4-(4-methylpiperazin-1-yl)aniline (185 mg, 0.969 mmol) and trifluoroacetic acid (147 mg, 1.29 mmol) in 2-butanol (10 mL). The reaction was stirred for 2 h at 100° C. The residue was purified by reverse phase flash column chromatography eluting with acetonitrile (50-70%) in water (0.4% formic acid) to afford the title compound as an orange solid (260 mg, 65%).

(ES, m/z): [M+H]⁺=614.3

### 5-ethynyl-N-[4-(4-methylpiperazin-1-yl)phenyl]-7-(1,3-thiazol-4-ylmethoxy)pyrido[2,3-d]pyrimidin-2-amine

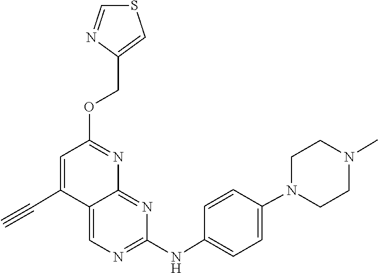 [SMILES: C#Cc1cc(OCc2cscn2)nc2nc(Nc3ccc(N4CCN(C)CC4)cc3)ncc12]

General Procedure 1 was applied to N-[4-(4-methylpiperazin-1-yl)phenyl]-7-(1,3-thiazol-4-ylmethoxy)-5-[2-(triisopropylsilyl)ethynyl]pyrido[2,3-d]pyrimidin-2-amine (220 mg, 0.358 mmol) and potassium fluoride (208 mg, 3.58 mmol) in DMF (2.5 mL), THF (2.5 mL) and water (200 uL). The resulting mixture was stirred for 1 h at 60° C. The residue was purified by reverse phase flash column chromatography eluting with acetonitrile (30-40%) in water (0.4% formic acid) to afford the title compound as a yellow solid (70.0 mg, 39%).

(ES, m/z): [M+H]⁺=458.2

¹H NMR (400 MHz, DMSO-d₆) δ 9.95 (s, 1H), 9.15 (d, J=1.8 Hz, 2H), 7.84 (d, J=2.0 Hz, 1H), 7.71 (s, 2H), 6.98 (s, 1H), 6.95 (d, J=9.1 Hz, 2H), 5.63 (s, 2H), 5.06 (s, 1H), 3.10 (t, J=5.0 Hz, 4H), 2.46 (t, J=5.0 Hz, 4H), 2.22 (s, 3H).

### Example 14

7-(cyclohexyloxy)-2-(methylthio)-5-((triisopropylsilyl)ethynyl)pyrido[2,3-d]pyrimidine

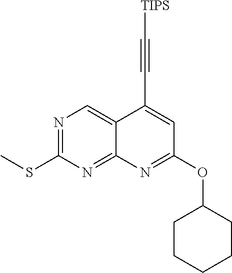

General Procedure 8 was applied to 2-(methylthio)-5-((triisopropylsilyl)ethynyl)pyrido[2,3-d]pyrimidin-7(8H)-one (1.00 g, 2.68 mmol) with cyclohexanol (560 μL, 5.36 mmol), triphenylphosphine (1.05 g, 4.01 mmol) and DEAD (0.641 mL, 4.01 mmol) in THF (7.0 mL). The crude material was purified by flash column chromatography eluting with ethyl acetate (0-2%) in 40-60 petroleum ether to yield the title compound as a pale yellow solid (451 mg, 0.991 mmol, 37%).

(ES, m/z): [M+H]⁺=456.4

### 7-(cyclohexyloxy)-2-(methylsulfonyl)-5-((triisopropylsilyl)ethynyl)pyrido[2,3-d]pyrimidine

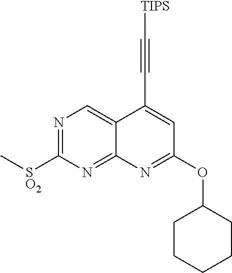

General Procedure 2 was applied to 7-(cyclohexyloxy)-2-(methylthio)-5-((triisopropylsilyl)ethynyl)pyrido[2,3-d]pyrimidine (400 mg, 0.878 mmol) with m-CPBA (454 mg, 2.63 mmol) in dichloromethane (9.0 mL). The crude title compound was carried forward without further purification (355 mg, 0.728 mmol, 83%).

(ES, m/z): [M+H]⁺=488.5

### 7-(cyclohexyloxy)-N-(4-(4-methylpiperazin-1-Y)phenyl)-5-((triisopropylsilyl)ethynyl)pyrido[2,3-d]pyrimidin-2-amine

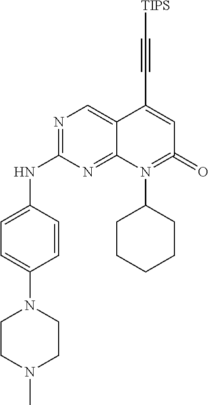

General Procedure 3 was applied to 7-(cyclohexyloxy)-2-(methylsulfonyl)-5-((triisopropylsilyl)ethynyl)pyrido[2,3-d]pyrimidine (350 mg, 0.718 mmol) with 4-(4-methylpiperazin-1-yl)aniline (206 mg, 1.08 mmol) and trifluoroacetic acid (82.0 μL, 1.08 mmol) in acetonitrile (7.0 mL). The crude material was purified by flash column chromatography eluting with methanol (0-20%) in dichloromethane to yield the title compound as an orange solid (357 mg, 0.596 mmol, 83%).

(ES, m/z): [M+H]⁺=599.7

### 7-(cyclohexyloxy)-5-ethynyl-N-(4-(4-methylpiperazin-1-yl)phenyl)pyrido[2,3-d]pyrimidin-2-amine

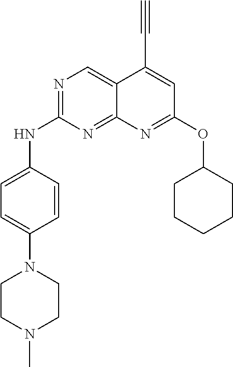 [SMILES: C#Cc1cc(OC2CCCCC2)nc2nc(Nc3ccc(N4CCN(C)CC4)cc3)ncc12]

General Procedure 1 was applied to 7-(cyclohexyloxy)-N-(4-(4-methylpiperazin-1-yl)phenyl)-5-((triisopropylsilyl)ethynyl)pyrido[2,3-d]pyrimidin-2-amine (150 mg, 0.250 mmol) with potassium fluoride (291 mg, 5.01 mmol) in DMF (1.25 mL). The crude material was purified by flash column chromatography eluting with methanol (0-20%) in dichloromethane to yield the title compound as a red solid (72.4 mg, 0.164 mmol, 66%).

(ES, m/z): [M+H]⁺=443.4

¹H NMR (500 MHz, Chloroform-d) δ 9.11 (s, 1H), 7.47 (d, J=8.4 Hz, 2H), 7.19 (s, 1H), 6.89 (d, J=9.0 Hz, 2H), 6.70 (s, 1H), 5.43-5.32 (m, 1H), 3.49 (s, 1H), 3.19 (t, J=5.0 Hz, 4H), 2.64 (t, J=4.9 Hz, 4H), 2.37 (s, 3H), 1.96 (t, J=7.5 Hz, 2H), 1.71 (dt, J=13.2, 4.9 Hz, 2H), 1.58-1.32 (m, 4H), 1.30-1.04 (m, 2H).

### Example 15

### 2-(methylthio)-7-((tetrahydro-2H-pyran-4-yl)oxy)-5-((triisopropvisilyl)ethynyl)pyrido[2,3-d]pyrimidine

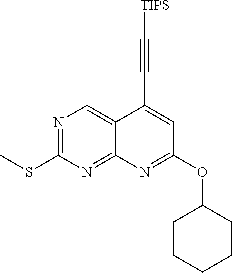

General Procedure 8 was applied to 2-(methylthio)-5-((triisopropylsilyl)ethynyl)pyrido[2,3-d]pyrimidin-7(8H)-one (1.50 g, 4.01 mmol) with tetrahydro-2H-pyran-4-ol (420 μL, 4.42 mmol), triphenylphosphine (2.11 g, 8.03 mmol) and DIAD (1.58 mL, 8.03 mmol) in THF (8.0 mL). The crude material was purified by flash column chromatography eluting with ethyl acetate (0-30%) in 40-60 petroleum ether to yield the title compound as a pale yellow solid (489 mg, 1.07 mmol, 27%).

(ES, m/z): [M+H]⁺=456.5 2-(methylsulfonyl)-7-((tetrahvdro-2H-pyran-4-yl)oxy)-5-((triisopropylsilyl)ethynyl)pyrido[2,3-d]pyrimidine

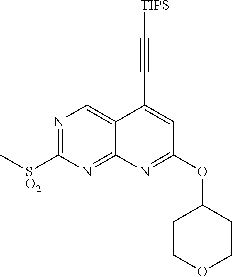

General Procedure 2 was applied to 2-(methylthio)-7-((tetrahydro-2H-pyran-4-yl)oxy)-5-((triisopropylsilyl)ethynyl)pyrido[2,3-d]pyrimidine (450 mg, 0.983 mmol) with m-CPBA (509 mg, 2.95 mmol) in dichloromethane (10 mL). The crude title compound was carried forward without further purification (433 mg, 0.885 mmol, 90%).

(ES, m/z): [M+H]⁺=490.4

### N-(4-(4-methylpiperazin-1-yl)phenyl)-7-((tetrahydro-2H-pyran-4-yl)oxy)-5-((triisopropylsilyl)ethynyl)pyrido[2,3-d]pyrimidin-2-amine

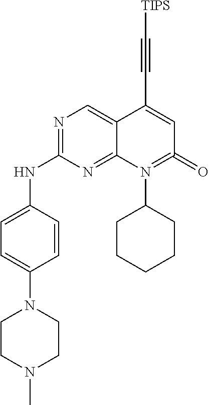

General Procedure 3 was applied to 2-(methylsulfonyl)-7-((tetrahydro-2H-pyran-4-yl)oxy)-5-((triisopropylsilyl)ethynyl)pyrido[2,3-d]pyrimidine (400 mg, 0.817 mmol) with 4-(4-methylpiperazin-1-yl)aniline (234 mg, 1.23 mmol) and trifluoroacetic acid (94.0 μL, 1.23 mmol) in acetonitrile (8.0 mL). The crude material was purified by flash column chromatography eluting with methanol (0-20%) in dichloromethane to yield the title compound as an orange solid (370 mg, 0.615 mmol, 75%).

(ES, m/z): [M+H]⁺=601.7

### 5-ethynyl-N-(4-(4-methylpiperazin-1-yl)phenyl)-7-((tetrahvdro-2H-pyran-4-yl)oxy)pyrido[2,3-d]pyrimidin-2-amine

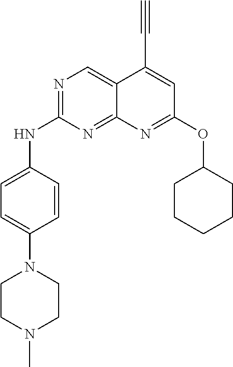 [SMILES: C#Cc1cc(OC2CCCCC2)nc2nc(Nc3ccc(N4CCN(C)CC4)cc3)ncc12]

General Procedure 1 was applied to N-(4-(4-methylpiperazin-1-yl)phenyl)-7-((tetrahydro-2H-pyran-4-yl)oxy)-5-((triisopropylsilyl)ethynyl)pyrido[2,3-d]pyrimidin-2-amine (150 mg, 0.249 mmol) with potassium fluoride (290 mg, 4.99 mmol) in DMF (1.25 mL). The crude material was purified by flash column chromatography eluting with methanol (0-20%) in dichloromethane to yield the title compound as an red solid (68.7 mg, 0.155 mmol, 62%).

(ES, m/z): [M+H]⁺=445.4

¹H NMR (500 MHz, Chloroform-d) δ 9.14 (s, 1H), 7.49 (d, J=8.4 Hz, 2H), 7.27 (s, 1H), 6.89 (d, J=8.9 Hz, 2H), 6.74 (s, 1H), 5.57 (dt, J=8.8, 4.5 Hz, 1H), 3.92 (dt, J=11.8, 4.4 Hz, 2H), 3.57 (ddd, J=12.0, 9.4, 2.9 Hz, 2H), 3.42 (d, J=3.4 Hz, 1H), 3.29 (t, J=5.1 Hz, 4H), 2.81 (s, 4H), 2.49 (s, 3H), 2.13-1.95 (m, 2H), 1.76 (dtd, J=13.2, 9.1, 4.1 Hz, 2H).

### Example 16

### 7-cyclopropoxy-2-(methylsulfanyl)-5-[2-(triisopropylsilyl)ethynyl]pyrido [2,3-d]pyrimidine

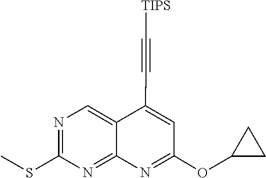

A solution of cyclopropanol (222 mg, 3.83 mmol, 3.0 eq.) in THF (5 mL) was treated with sodium hydride (45.9 mg, 1.91 mmol, 1.5 eq.) at 0° C. then 7-chloro-2-(methylsulfanyl)-5-[2-(triisopropylsilyl)ethynyl]pyrido[2,3-d]pyrimidine (500 mg, 1.28 mmol, 1.0 eq.) was added portionwise. The resulting mixture was stirred for 2 h at 0° C. then extracted with ethyl acetate (2×30 mL). The combined organic layers were washed with brine (3×20 mL), dried (Na₂SO₄) and concentrated under reduced pressure. The residue was purified by flash column chromatography eluting with ethyl acetate (5%) in 40-60 petroleum ether to afford the title compound as a light yellow oil (520 mg, 99%).

(ES, m/z): [M+H]⁺=414.2

### 7-cyclopropoxy-2-methanesulfonyl-5-[2-(triisopropylsilyl)ethynyl]pyrido[2,3-d]pyrimidine

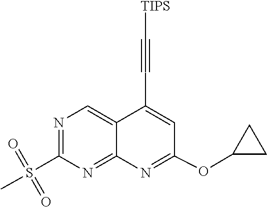

General Procedure 2 was applied to 7-cyclopropoxy-2-(methylsulfanyl)-5-[2-(triisopropylsilyl)ethynyl]pyrido [2,3-d]pyrimidine (424 mg, 1.03 mmol) and m-CPBA (389 mg, 2.25 mmol) in dichloromethane (1 mL) to yield the title compound as a light yellow oil (450 mg, 99%).

(ES, m/z): [M+H]⁺=446.2

### 7-cyclopropoxy-N-[4-(4-methylpiperazin-1-yl)phenyl]-5-[2-(triisopropylsilyl)ethynyl]pyrido[2,3-d]pyrimidin-2-amine

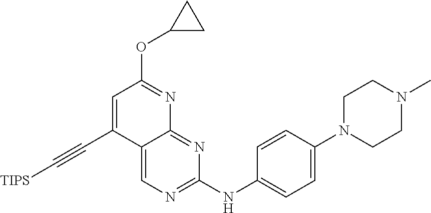

General Procedure 3 was applied to 7-cyclopropoxy-2-methanesulfonyl-5-[2-(triisopropylsilyl)ethynyl]pyrido[2,3-d]pyrimidine (490 mg, 1.10 mmol), 4-(4-methylpiperazin-1-yl)aniline (315 mg, 1.65 mmol) and trifluoroacetic acid (251 mg, 2.20 mmol) in 2-butanol (5 mL). The resulting mixture was stirred for 3 h at 100° C. to yield the title compound as an orange red solid (130 mg, 21%).

(ES, m/z): [M+H]⁺=557.3

### 7-cyclopropoxy-5-ethynyl-N-[4-(4-methylpiperazin-1-yl)phenyl]pyrido[2,3-d]pyrimidin-2-amine

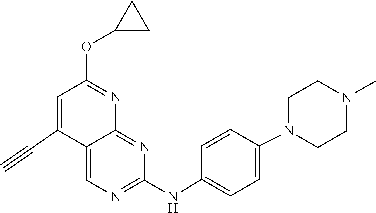 [SMILES: C#Cc1cc(OC2CC2)nc2nc(Nc3ccc(N4CCN(C)CC4)cc3)ncc12]

General Procedure 1 was applied to 7-cyclopropoxy-N-[4-(4-methylpiperazin-1-yl)phenyl]-5-[2-(triisopropylsilyl)ethynyl]pyrido[2,3-d]pyrimidin-2-amine (143 mg, 0.257 mmol) and potassium fluoride (149 mg, 2.57 mmol) in THF (2.86 mL), DMF (2.86 mL) and water (143 uL). The reaction was stirred for 2 h at 60° C. to yield the title compound as a yellow solid (35.0 mg, 34%).

(ES, m/z): [M+H]⁺=401.2

¹H NMR (400 MHz, DMSO-d₆) δ 9.94 (s, 1H), 9.14 (s, 1H), 8.23 (s, 1H), 7.70 (d, J=8.4 Hz, 2H), 6.99-6.82 (m, 3H), 5.06 (s, 1H), 4.55-4.45 (m, 1H), 3.10 (t, J=5.0 Hz, 4H), 2.48 (d, J=4.9 Hz, 4H), 2.24 (s, 3H), 0.85 (q, J=6.8, 5.9 Hz, 2H), 0.76 (h, J=3.6, 3.0 Hz, 2H).

### Example 17

### 7-isopropoxy-2-(methylthio)-5-((triisopropylsilyl)ethynyl)pyrido[2,3-d]pyrimidine

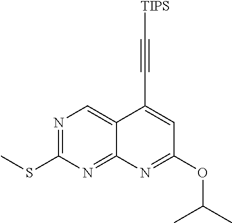

A solution of isopropyl alcohol (30.7 mg, 0.510 mmol, 2.0 eq.) in THF (2 mL) was treated with sodium hydride (15.3 mg, 0.383 mmol, 1.5 eq.) at 0° C., then stirred for 30 min at room temperature. 7-chloro-2-(methylsulfanyl)-5-[2-(triisopropylsilyl) ethynyl]pyrido[2,3-d]pyrimidine (100 mg, 0.255 mmol, 1.0 eq.) was added portionwise at 0° C. The resulting mixture was stirred for 2 days at room temperature to yield the title compound.

(ES, m/z): [M+H]⁺=416.2

### 7-isopropoxy-2-methanesulfonyl-5-[2-(triisopropylsilyl)ethynyl]pyrido[2,3-d]pyrimidine

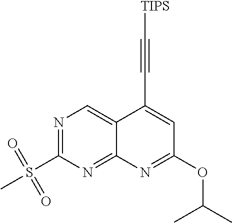

General Procedure 2 was applied to 7-isopropoxy-2-(methylsulfanyl)-5-[2-(triisopropylsilyl)ethynyl]pyrido[2,3-d]pyrimidine (403 mg, 0.970 mmol) and m-CPBA (368 mg, 2.13 mmol) in dichloromethane (4 mL) to yield the title compound as a light yellow oil (403 mg, 93%).

(ES, m/z): [M+H]⁺=448.2

### 7-isopropoxy-N-[4-(4-methylpiperazin-1-yl)phenyl]-5-[2-(triisopropylsilyl)ethynyl]pyrido[2,3-d]pyrimidin-2-amine

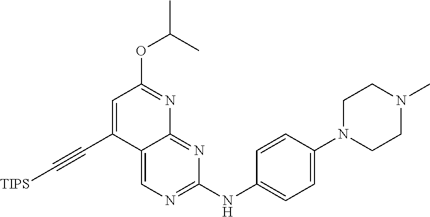

General Procedure 3 was applied to 7-isopropoxy-2-methanesulfonyl-5-[2-(triisopropylsilyl)ethynyl]pyrido [2,3-d]pyrimidine (379 mg, 0.847 mmol), trifluoroacetic acid (193 mg, 1.69 mmol) and 4-(4-methylpiperazin-1-yl)aniline (243 mg, 1.27 mmol) in 2-butanol (1 mL) to yield the title compound as an orange solid (262 mg, 55%).

(ES, m/z): [M+H]⁺=559.4

### 5-ethynyl-7-isopropoxy-N-[4-(4-methylpiperazin-1-yl)phenyl]pyrido[2,3-d]pyrimidin-2-amine

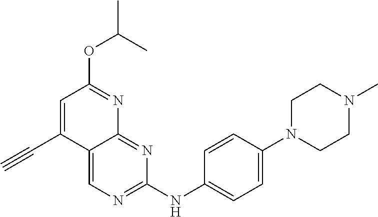 [SMILES: C#Cc1cc(OC(C)C)nc2nc(Nc3ccc(N4CCN(C)CC4)cc3)ncc12]

General Procedure 1 was applied to 7-isopropoxy-N-[4-(4-methylpiperazin-1-yl)phenyl]-5-[2-(triisopropylsilyl)ethynyl]pyrido[2,3-d]pyrimidin-2-amine (210 mg, 0.376 mmol) and potassium fluoride (218 mg, 3.76 mmol) in THF (3 mL), DMF (3 mL) and water. The reaction was stirred for 2 h at room temperature. The residue was purified by reverse phase flash column chromatography eluting with acetonitrile (10-50%) in water (0.1% trifluoroacetic acid) in water to yield the title compound as a yellow solid (62.3 mg, 41%).

(ES, m/z): [M+H]⁺=403.2

¹H NMR (400 MHz, DMSO-d₆) δ 9.89 (s, 1H), 9.10 (s, 1H), 7.69 (s, 2H), 6.99-6.89 (m, 2H), 6.83 (s, 1H), 5.54-5.45 (m, 1H), 5.02 (s, 1H), 3.09 (t, J=5.0 Hz, 4H), 2.46 (t, J=5.0 Hz, 4H), 2.22 (s, 3H), 1.35 (d, J=6.2 Hz, 6H), 1.24 (s, 2H).

### Example 18

### 7-cyclobutoxy-2-(methylsulfanyl)-5-[2-(triisopropylsilyl)ethynyl]pyrido[2,3-d]pyrimidine

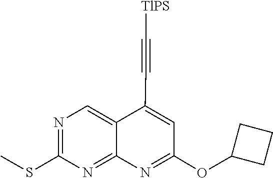

A solution of 7-chloro-2-(methylsulfanyl)-5-[2-(triisopropylsilyl)ethynyl]pyrido[2,3-d]pyrimidine (1.10 g, 2.81 mmol, 1.0 eq.) in dioxane (11 mL) was treated with potassium tert-butoxide (472 mg, 4.21 mmol, 1.5 eq.) for 30 minutes at room temperature followed by the addition of cyclobutanol (303 mg, 4.21 mmol, 1.5 eq.) portionwise. The resulting mixture was stirred for 2 h at room temperature then extracted with ethyl acetate (2×50 mL). The combined organic layers were washed with brine (3×100 mL), dried (Na₂SO₄) and concentrated under reduced pressure. The residue was purified by flash column chromatography eluting with ethyl acetate (100%) to afford the title compound as a brown oil (1.06 g, 88%).

(ES, m/z): [M+H]⁺=428.2

### 7-cyclobutoxy-2-methanesulfonyl-5-[2-(triisopropylsilyl)ethynyl]pyrido[2,3-d]pyrimidine

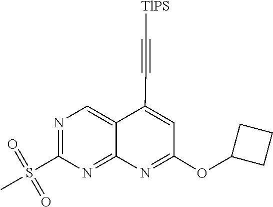

General Procedure 2 was applied to 7-cyclobutoxy-2-(methylsulfanyl)-5-[2-(triisopropylsilyl)ethynyl]pyrido[2,3-d]pyrimidine (1.03 mg, 2.40 mmol) and m-CPBA (912 mg, 5.28 mmol) in dichloromethane (10 mL) to yield the title compound as a light yellow solid (1.04 g, 94%).

(ES, m/z): [M+H]⁺=459.2

### 7-cyclobutoxy-N-[4-(4-methylpiperazin-1-yl)phenyl]-5-[2-(triisopropylsilylI)ethynyl]pyrido[2,3-d]pyrimidin-2-amine

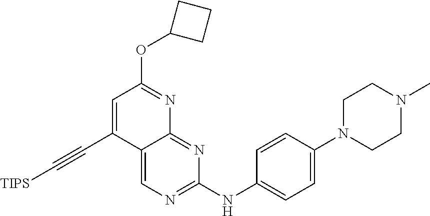

General Procedure 3 was applied to 7-cyclobutoxy-2-methanesulfonyl-5-[2-(triisopropylsilyl)ethynyl]pyrido [2,3-d]pyrimidine (936 mg, 2.04 mmol), 4-(4-methylpiperazin-1-yl)aniline (584 mg, 3.05 mmol) and trifluoroacetic acid (464 mg, 4.07 mmol) in 2-butanol (1 mL). The resulting mixture was stirred for 2 h at 100° C. This resulted in the title compound as an orange red solid (467 mg, 40%).

(ES, m/z): [M+H]⁺=571.4

### 7-cyclobutoxy-5-ethynyl-N-[4-(4-methylpiperazin-1-yl)phenyl]pyrido[2,3-d]pyrimidin-2-amine

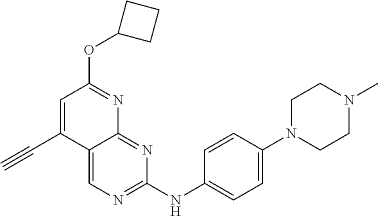 [SMILES: C#Cc1cc(OC2CCC2)nc2nc(Nc3ccc(N4CCN(C)CC4)cc3)ncc12]

General Procedure 1 was applied to 7-cyclobutoxy-N-[4-(4-methylpiperazin-1-yl)phenyl]-5-[2-(triisopropylsilyl)ethynyl]pyrido[2,3-d]pyrimidin-2-amine (250 mg, 0.438 mmol) and potassium fluoride (254 mg, 4.38 mmol) in THF (1 mL), DMF (1 mL) and water (1 mL). The reaction was stirred for 2 h at room temperature. The residue was purified by reverse phase flash column chromatography eluting with acetonitrile (10-50%) in water (0.1% trifluoroacetic acid) to yield the title compound as a yellow solid (89.0 mg, 49%).

(ES, m/z): [M+H]⁺=413.2

¹H NMR (400 MHz, DMSO-d₆) δ 9.90 (s, 1H), 9.11 (s, 1H), 7.69 (s, 2H), 6.97-6.91 (m, 2H), 6.87 (s, 1H), 5.34 (p, J=7.4 Hz, 1H), 5.04 (s, 1H), 3.10 (t, J=5.0 Hz, 4H), 2.48-2.41 (m, 6H), 2.23 (s, 3H), 2.12 (dtd, J=12.3, 9.8, 7.7 Hz, 2H), 1.86-1.75 (m, 1H), 1.69 (tdd, J=10.4, 7.8, 2.4 Hz, 1H), 1.23 (s, 1H).

### Example 19

### 7-(cyclopropylmethoxy)-2-(methylsulfanyl)-5-[2-(triisopropylsilyl)ethynyl]pyrido[2,3-d]pyrimidine

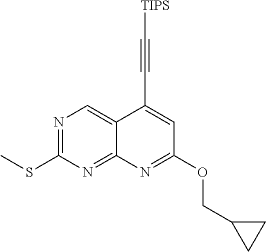

A solution of cyclopropylmethanol (276 mg, 3.83 mmol, 1.5 eq.) in dioxane (1 mL) was treated with potassium tert-butoxide (429 mg, 3.83 mmol, 1.5 eq.) followed by 7-chloro-2-(methylsulfanyl)-5-[2-(triisopropylsilyl)ethynyl]pyrido[2,3-d]pyrimidine (1.00 g, 2.55 mmol, 1.0 eq.) portionwise. The resulting mixture was stirred for 2 h at room temperature then extracted with ethyl acetate (2×100 mL). The combined organic layers were washed with brine (3×100 mL), dried (Na₂SO₄) and concentrated under reduced pressure. The residue was purified by flash column chromatography eluting with ethyl acetate (5%) in 40-60 petroleum ether to afford the title compound as a light yellow oil (850 mg, 78%).

(ES, m/z): [M+H]⁺=428.2

### 7-isopropoxy-2-methanesulfonyl-5-[2-(triisopropylsilyl)ethynyl]pyrido[2,3-d]pyrimidine

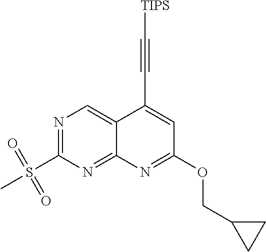

General Procedure 2 was applied to 7-isopropoxy-2-(methylsulfanyl)-5-[2-(triisopropylsilyl)ethynyl]pyrido[2,3-d]pyrimidine (795 mg, 1.91 mmol) and m-CPBA (726 mg, 4.21 mmol) in dichloromethane (1 mL) to afford the title compound as a light yellow oil (813 mg, 95%).

(ES, m/z): [M+H]⁺=460.2

### 7-(cyclopropylmethoxy)-N-[4-(4-methylpiperazin-1-yl)phenyl]-5-[2-(triisopropylsilyl)ethynyl]pyrido[2,3-d]pyrimidin-2-amine

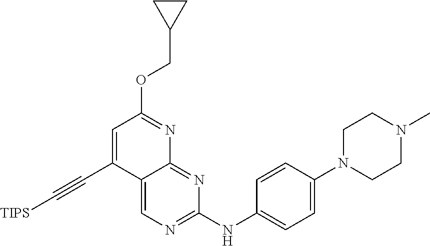

General Procedure 3 was applied to 7-(cyclopropylmethoxy)-2-methanesulfonyl-5-[2-(triisopropylsilyl)ethynyl]pyrido[2,3-d]pyrimidine (678 mg, 1.48 mmol), trifluoroacetic acid (336 mg, 2.95 mmol) and 4-(4-methylpiperazin-1-yl)aniline (423 mg, 2.21 mmol) in 2-butanol (7 mL). The resulting mixture was stirred for 3 h at 100° C. to yield the title compound as an orange red solid (496 mg, 59%).

(ES, m/z): [M+H]⁺=471.4

### 7-cyclopropoxy-5-ethynyl-N-[4-(4-methylpiperazin-1-yl)phenyl]pyrido[2,3-d]pyrimidin-2-amine

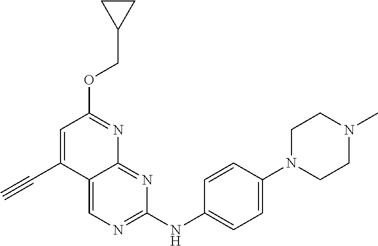 [SMILES: C#Cc1cc(OCC2CC2)nc2nc(Nc3ccc(N4CCN(C)CC4)cc3)ncc12]

General Procedure 1 was applied to 7-cyclopropoxy-N-[4-(4-methylpiperazin-1-yl)phenyl]-5-[2-(triisopropylsilyl)ethynyl]pyrido[2,3-d]pyrimidin-2-amine (250 mg, 0.449 mmol) and potassium fluoride (261 mg, 4.49 mmol) in THF (2 mL), DMF (2 mL) and water (250 uL). The reaction was stirred for 2 h at 60° C. to yield the title compound as an orange solid (106 mg, 59%).

(ES, m/z): [M+H]⁺=415.2

¹H NMR (400 MHz, DMSO-d₆) δ 9.89 (s, 1H), 9.11 (s, 1H), 7.69 (d, J=8.3 Hz, 2H), 6.99-6.84 (m, 3H), 5.02 (d, J=10.7 Hz, 1H), 4.26 (d, J=7.2 Hz, 2H), 3.09 (q, J=5.1 Hz, 4H), 2.46 (t, J=5.0 Hz, 4H), 2.23 (s, 3H), 1.37-1.19 (m, 1H), 0.64-0.49 (m, 2H), 0.44-0.27 (m, 2H).

### Example 20

### 7-(benzyloxy)-2-(methylsulfanyl)-5-[2-(triisopropylsilyl)ethynyl]pyrido [2,3-d]pyrimidine

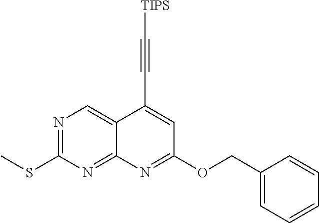

A solution of 7-chloro-2-(methylsulfanyl)-5-[2-(triisopropylsilyl)ethynyl]pyrido[2,3-d]pyrimidine (800 mg, 2.04 mmol, 1.0 eq.) in dioxane (1 mL) was treated with potassium tert-butoxide (229 mg, 2.04 mmol, 1.0 eq.) for 30 min at room temperature followed by the addition of benzyl alcohol (331 mg, 3.06 mmol, 1.5 eq.) portionwise. The resulting mixture was stirred for 2 h at room temperature then extracted with ethyl acetate (3×50 mL). The combined organic layers were washed with brine (3×100 mL), dried (Na₂SO₄) and concentrated under reduced pressure. The residue was purified by flash column chromatography eluting with ethyl acetate (5-10%) in 40-60 petroleum ether to afford the title compound as a white solid (883 mg, 93%).

(ES, m/z): [M+H]⁺=464.2

### 7-(benzyloxy)-2-methanesulfonyl-5-[2-(triisopropylsilyl)ethynyl]pyrido[2,3-d]pyrimidine

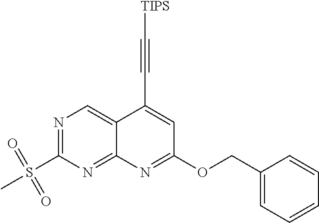

General Procedure 2 was applied to 7-(benzyloxy)-2-(methylsulfanyl)-5-[2-(triisopropylsilyl)ethynyl]pyrido[2,3-d]pyrimidine (730 mg, 1.57 mmol) and m-CPBA (598 mg, 3.46 mmol) in dichloromethane (8 mL) to yield the title compound as a white solid (763 mg, 98%).

(ES, m/z): [M+H]⁺=496.2

### 7-(benzyloxy)-N-[4-(4-methylpiperazin-1-yl)phenyl]-5-[2-(triisopropylsilyl)ethynyl]pyrido[2,3-d]pyrimidin-2-amine

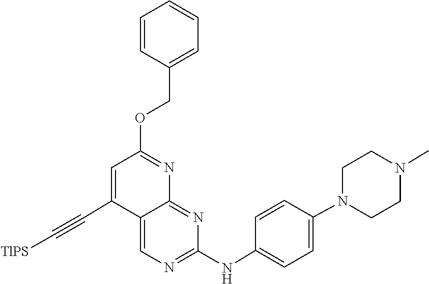

General Procedure 3 was applied to 7-(benzyloxy)-2-methanesulfonyl-5-[2-(triisopropylsilyl)ethynyl]pyrido [2,3-d]pyrimidine (763 mg, 1.54 mmol), trifluoroacetic acid (351 mg, 3.08 mmol) and 4-(4-methylpiperazin-1-yl)aniline (442 mg, 2.31 mmol) in 2-butanol (1 mL). The resulting mixture was stirred for 2 h at 100° C. The residue was purified by reverse phase flash column chromatography eluting with acetonitrile (10-50%) in water to yield the title compound as an orange solid (436 mg, 47%).

(ES, m/z): [M+H]⁺=606.4

### 7-(benzyloxy)-5-ethynyl-N-[4-(4-methylpiperazin-1-yl)phenyl]pyrido[2,3-d]pyrimidin-2-amine

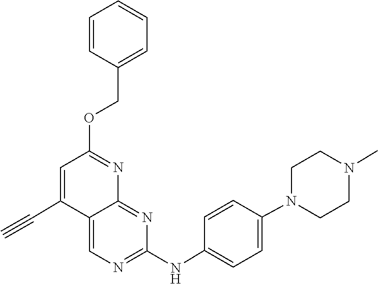 [SMILES: C#Cc1cc(OCc2ccccc2)nc2nc(Nc3ccc(N4CCN(C)CC4)cc3)ncc12]

General Procedure 1 was applied to 7-cyclobutoxy-N-[4-(4-methylpiperazin-1-yl)phenyl]-5-[2-(triisopropylsilyl)ethynyl]pyrido[2,3-d]pyrimidin-2-amine (250 mg, 0.438 mmol) and potassium fluoride (239 mg, 4.12 mmol) in THF (2 mL), DMF (2 mL) and water.

The reaction was stirred for 2 h at room temperature. The residue was purified by reverse phase flash column chromatography eluting with methanol (50-80%) in water to yield the title compound as a yellow solid (110 mg, 59%).

(ES, m/z): [M+H]⁺=451.2

¹H NMR (400 MHz, DMSO-d6) δ 10.01 (s, 1H), 9.16 (s, 1H), 7.77 (s, 2H), 7.52 (d, J=6.9 Hz, 2H), 7.48-7.32 (m, 3H), 7.01 (t, J=4.5 Hz, 3H), 5.52 (s, 2H), 5.07 (s, 1H), 3.14 (s, 4H), 2.70 (s, 3H).

### Example 21

### 2-(methylsulfanyl)-7-(2-phenylethoxy)-5-[2-(triisopropylsilyl)ethynyl]pyrido[2,3-d]pyrimidine

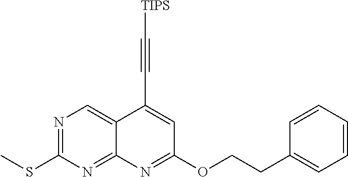

A solution of 7-chloro-2-(methylsulfanyl)-5-[2-(triisopropylsilyl)ethynyl]pyrido[2,3-d]pyrimidine (1.00 g, 2.55 mmol, 1.0 eq.) in dioxane (1 mL) was treated with potassium tert-butoxide (286 mg, 2.55 mmol, 1.0 eq.) followed by the addition of 2-phenyl-ethanol (467 mg, 3.83 mmol, 1.5 eq.) portionwise. The resulting mixture was stirred for 2 h at room temperature then extracted with ethyl acetate (3×50 mL). The combined organic layers were washed with brine (3×100 mL), dried (Na₂SO₄) and concentrated under reduced pressure. The residue was purified by flash column chromatography eluting with ethyl acetate(5-10%) in 40-60 petroleum ether to afford the title compound as a light pink solid (669 mg, 55%).

(ES, m/z): [M+H]⁺=478.2

### 2-methanesulfonyl-7-(2-phenylethoxy)-5-[2-(triisopropylsilyl)ethynyl]pyrido[2,3-d]pyrimidine

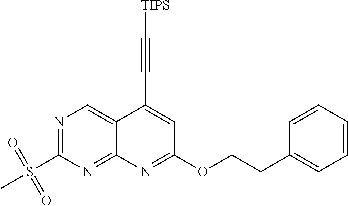

General Procedure 2 was applied to 2-(methylsulfanyl)-7-(2-phenylethoxy)-5-[2-(triisopropylsilyl)ethynyl]pyrido[2,3-d]pyrimidine (650 mg, 1.36 mmol) and m-CPBA (516 mg, 2.99 mmol) in dichloromethane (7 mL) to yield the title compound as a light yellow solid (636 mg, 92%).

(ES, m/z): [M+H]⁺=510.2

### N-[4-(4-methylpiperazin-1-yl)phenyl]-7-(2-phenylethoxy)-5-[2-(triisopropylsilylI)ethynyl]pyrido[2,3-d]pyrimidin-2-amine

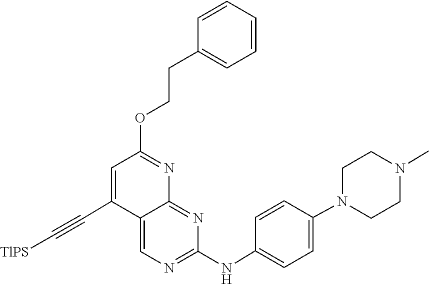

General Procedure 3 was applied to 2-methanesulfonyl-7-(2-phenylethoxy)-5-[2-(triisopropylsilyl)ethynyl]pyrido[2,3-d]pyrimidine (557 mg, 1.09 mmol), trifluoroacetic acid (249 mg, 2.18 mmol) and 4-(4-methylpiperazin-1-yl)aniline (314 mg, 1.64 mmol) in 2-butanol (10 mL). The reaction was stirred at 100° C. for 2 h to yield the title compound as an orange solid (212 mg, 31%).

(ES, m/z): [M+H]⁺=621.4

### 5-ethynyl-N-[4-(4-methylpiperazin-1-yl)phenyl]-7-(2-phenylethoxy)pyrido[2,3-d]pyrimidin-2-amine

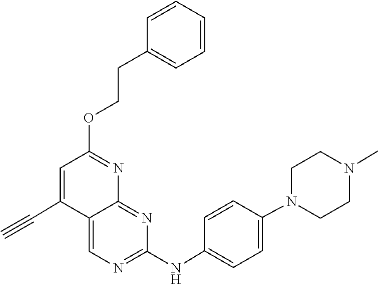 [SMILES: C#Cc1cc(OCCc2ccccc2)nc2nc(Nc3ccc(N4CCN(C)CC4)cc3)ncc12]

General Procedure 1 was applied to N-[4-(4-methylpiperazin-1-yl)phenyl]-7-(2-phenylethoxy)-5-[2-(triisopropylsilyl)ethynyl]pyrido[2,3-d]pyrimidin-2-amine (210 mg, 0.338 mmol) and potassium fluoride (196 mg, 3.38 mmol) in THE (2 mL), DMF (2 mL) and water (210 uL). The reaction was stirred for 2 h at room temperature to yield the title compound as an orange solid (138 mg, 88%).

(ES, m/z): [M+H]⁺=465.2

¹H NMR (400 MHz, DMSO-d₆) δ 9.96 (s, 1H), 9.13 (s, 1H), 7.74 (d, J=8.2 Hz, 2H), 7.38-7.29 (m, 4H), 7.24 (td, J=6.3, 2.0 Hz, 1H), 7.01-6.95 (m, 2H), 6.90 (s, 1H), 5.05 (s, 1H), 4.63 (t, J=6.9 Hz, 2H), 3.21 (s, 4H), 3.10 (t, J=6.9 Hz, 3H), 2.85 (s, 4H).

### Example 22

### 2-(methylsulfanyl)-7-(3-phenylPropoxy)-5-[2-(triisoprooylsilyl)ethynyl]pyrido[2,3-d]pyrimidine

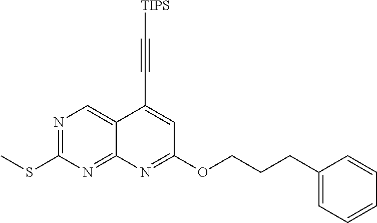

A solution of 7-chloro-2-(methylsulfanyl)-5-[2-(triisopropylsilyl)ethynyl]pyrido[2,3-d]pyrimidine (1.00 g, 2.55 mmol, 1.0 eq.) in dioxane (10 mL) was treated with potassium tert-butoxide (286 mg, 2.55 mmol, 1.0 eq.) followed by the addition of 3-phenylpropan-1-ol (521 mg, 3.83 mmol, 1.5 eq.) portionwise at 0° C. The resulting mixture was stirred for 2 h at room temperature then extracted with ethyl acetate (3×50 mL). The combined organic layers were washed with brine (3×100 mL), dried (Na₂SO₄) and concentrated under reduced pressure. The residue was purified by flash column chromatography eluting with ethyl acetate (0-5%) in 40-60 petroleum ether to afford the title compound as a light yellow oil (872 mg, 69%).

(ES, m/z): [M+H]⁺=492.2

### 2-methanesulfonyl-7-(3-phenylpropoxy)-5-[2-(triisopropylsilyl)ethynyl]pyrido[2,3-d]pyrimidine

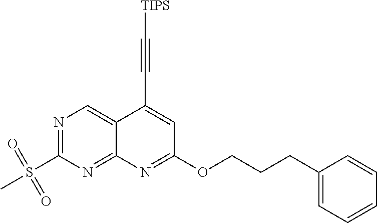

General Procedure 2 was applied to 2-(methylsulfanyl)-7-(3-phenylpropoxy)-5-[2-(triisopropylsilyl)ethynyl]pyrido[2,3-d]pyrimidine (872 mg, 1.77 mmol) and m-CPBA (673 mg, 3.90 mmol) in dichloromethane (1 mL) to yield the title compound as a light yellow oil (815 mg, 88%).

(ES, m/z): [M+H]⁺=524.2

### N-[4-(4-methylpiperazin-1-yl)phenyl]-7-(3-phenylpropoxy)-5-[2-(triisopropylsilyl)ethynyl]pyrido[2,3-d]pyrimidin-2-amine

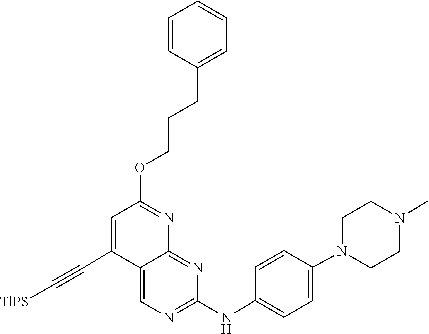

General Procedure 3 was applied to 2-methanesulfonyl-7-(3-phenylpropoxy)-5-[2-(triisopropylsilyl)ethynyl]pyrido[2,3-d]pyrimidine (788 mg, 1.50 mmol), trifluoroacetic acid (343 mg, 3.01 mmol) and 4-(4-methylpiperazin-1-yl)aniline (432 mg, 2.26 mmol) in 2-butanol (8 mL). The resulting mixture was stirred for 2 h at 100° C. The residue was purified by reverse phase flash column chromatography eluting with methanol (50-85%) in water to yield the title compound as an orange red solid (551 mg, 58%).

(ES, m/z): [M+H]⁺=635.4

### 5-ethynyl-N-[4-(4-methylpiperazin-1-yl)phenyl]-7-(3-phenylpropoxy)pyrido[2,3-d]pyrimidin-2-amine

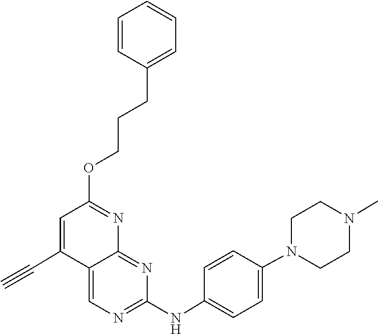 [SMILES: C#Cc1cc(OCCCc2ccccc2)nc2nc(Nc3ccc(N4CCN(C)CC4)cc3)ncc12]

General Procedure 1 was applied to N-[4-(4-methylpiperazin-1-yl)phenyl]-7-(3-phenylpropoxy)-5-[2-(triisopropylsilyl)ethynyl]pyrido[2,3-d]pyrimidin-2-amine (250 mg, 0.394 mmol) and potassium fluoride (228 mg, 3.94 mmol) in THE (3 mL), DMF (3 mL) and water (250 uL). The reaction was stirred for 2 h at room temperature. The residue was purified by reverse phase flash column chromatography eluting with methanol (20-45%) in water to yield the title compound as an orange solid (126 mg, 67%).

(ES, m/z): [M+H]⁺=479.2

¹H NMR (400 MHz, DMSO-d₆) δ 9.92 (s, 1H), 9.12 (s, 1H), 7.71 (d, J=7.4 Hz, 2H), 7.36-7.16 (m, 5H), 7.00-6.89 (m, 3H), 5.05 (s, 1H), 4.42 (t, J=6.5 Hz, 2H), 3.15 (s, 4H), 2.76 (dd, J=8.6, 6.6 Hz, 2H), 2.63 (s, 4H), 2.35 (s, 3H), 2.08 (dq, J=8.7, 6.5 Hz, 2H).

### Example 23

### 7-(cyclopentylmethoxy)-2-(methylsulfanyl)-5-[2-(triisopropylsilyl)ethynyl]pyrido[2,3-d]pyrimidine

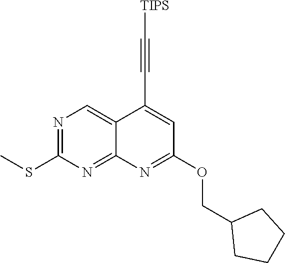

General Procedure 8 was applied to 2-(methylsulfanyl)-5-[2-(triisopropylsilyl)ethynyl]-8H-pyrido[2,3-d]pyrimidin-7-one (2.00 g, 5.35 mmol), cyclopentanemethanol (590 mg, 5.89 mmol), triphenylphosphine (2.81 g, 10.7 mmol) and DIAD (2.17 g, 10.7 mmol) in THE (10 mL). The residue was purified by reverse phase flash column chromatography eluting with acetonitrile (95-100%) in water (0.4% formic acid) to afford the title compound as a white solid (1.87 g, 77%).

(ES, m/z): [M+H]r=456.2 6-bromo-7-(cyclopentylmethoxy)-2-(methylsulfanyl)-5-[2-(triisopropylsilyl)ethynyl]pyrido[2,3-d]pyrimidine

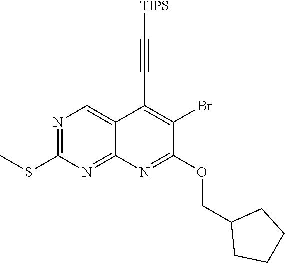

A mixture of 8-(cyclopentylmethyl)-2-(methylsulfanyl)-5-[2-(triisopropylsilyl)ethynyl]pyrido[2,3-d]pyrimidin-7-one (1.37 g, 3.01 mmol, 1.0 eq.), N-bromosuccinimide (804 mg, 4.52 mmol, 1.5 eq.) and acetic acid (36.2 mg, 0.603 mmol, 0.2 eq.) in acetonitrile (30 mL) was stirred for 2 h at room temperature. The residue was purified by reverse phase flash column chromatography eluting with acetonitrile (95-100%) in water (0.4% formic acid) to afford the title compound as a yellow oil (680 mg, 42%).

(ES, m/z): [M+H]⁺=534.1

### 7-(cyclopentylmethoxy)-6-methyl-2-(methylsulfanyl)-5-[2-(triisopropylsilyl)ethynyl]pyrido[2,3-d]pyrimidine

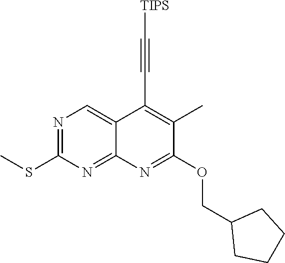

A mixture of 6-bromo-7-(cyclopentylmethoxy)-2-(methylsulfanyl)-5-[2-(triisopropylsilyl)ethynyl]pyrido[2,3-d]pyrimidine (480 mg, 0.898 mmol, 1.0 eq.), methylboronic acid (107 mg, 1.79 mmol, 2.0 eq.), potassium carbonate (248 mg, 1.79 mmol, 2.0 eq.) and [1,1′-bis(diphenylphosphino)ferrocene]dichloropalladium(II) (65.7 mg, 90.0 μmol, 0.1 eq.) in dioxane (4 mL) and water (1.6 mL) was stirred for 12 h at 70° C. The residue was purified by reverse phase flash column chromatography eluting with acetonitrile (100%) to afford the title compound as a yellow solid (218 mg, 52%).

(ES, m/z): [M+H]⁺=470.2

### 7-(cyclopentylmethoxy)-2-methanesulfonyl-6-methyl-5-[2-(triisopropylsilyl)ethynyl]pyrido[2,3-d]pyrimidine

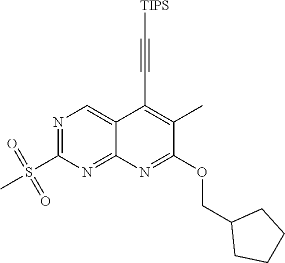

General Procedure 2 was applied to 7-(cyclopentylmethoxy)-6-methyl-2-(methylsulfanyl)-5-[2-(triisopropylsilyl) ethynyl]pyrido[2,3-d]pyrimidine (220 mg, 0.468 mmol) and m-CPBA (178 mg, 1.03 mmol) in dichloromethane (8 mL) to afford the title compound as a yellow solid (230 mg, 98%), which was used without further purification.

(ES, m/z): [M+H]⁺=502.2

### 8-(cyclopentylmethyl)-6-methyl-2-{[4-(4-methylpiperazin-1-yl)phenyl]amino}-5-[2-(triisopropylsilylI)ethynyl]pyrido[2,3-d]pyrimidin-7-one

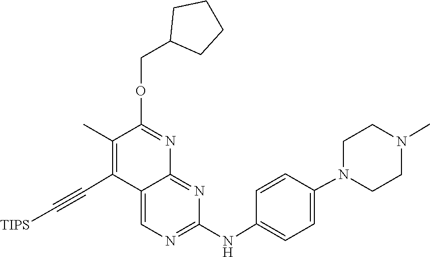

General Procedure 3 was applied to 7-(cyclopentylmethoxy)-2-methanesulfonyl-6-methyl-5-[2-(triisopropylsilyl)ethynyl]pyrido[2,3-d]pyrimidine (230 mg, 0.458 mmol), 4-(4-methylpiperazin-1-yl)aniline (263 mg, 1.37 mmol) and trifluoroacetic acid (261 mg, 2.29 mmol) in 2-butanol (5 mL). The reaction was stirred overnight at 100° C. The residue was purified by reverse phase flash column chromatography eluting with acetonitrile (60-70%) in water (0.4% formic acid) to afford the title compound as a yellow solid (170 mg, 61%).

(ES, m/z): [M+H]⁺=613.3

### 7-(cyclopentylmethoxy)-5-ethynyl-6-methyl-N-[4-(4-methylpiperazin-1-yl)phenyl]pyrido[2,3-d]pyrimidin-2-amine

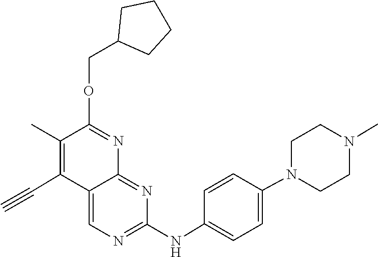 [SMILES: C#Cc1c(C)c(OCC2CCCC2)nc2nc(Nc3ccc(N4CCN(C)CC4)cc3)ncc12]

General Procedure 1 was applied to 7-(cyclopentylmethoxy)-6-methyl-N-[4-(4-methylpiperazin-1-yl)phenyl]-5-[2-(triisopropylsilyl)ethynyl]pyrido[2,3-d]pyrimidin-2-amine (140 mg, 0.228 mmol) and potassium fluoride (133 mg, 2.28 mmol) in DMF (1 mL), THF (1 mL) and water (100 uL). The reaction was stirred for 2 h at 60° C. The residue was purified by reverse phase flash column chromatography eluting with acetonitrile in water (0.1% formic acid) to afford the title compound as a yellow solid (71 mg, 67%).

(ES, m/z): [M+H]⁺=457.2

¹H NMR (400 MHz, DMSO-d₆) δ 9.92 (s, 1H), 8.74 (s, 1H), 7.62 (d, J=8.6 Hz, 2H), 6.91 (d, J=8.8 Hz, 2H), 5.21 (s, 1H), 4.24 (d, J=7.5 Hz, 2H), 3.08 (t, J=4.9 Hz, 4H), 2.46 (t, J=4.9 Hz, 5H), 2.22 (s, 6H), 1.66-1.51 (m, 4H), 1.49-1.40 (m, 2H), 1.38-1.28 (m, 2H).

### Example 24

### N,N-dimethyl-2-(methylsulfanyl)-5-[2-(triisopropylsilyl)ethynyl]pyrido[2,3-d]pyrimidin-7-amine

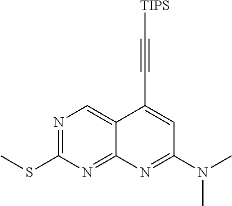

A solution of 7-chloro-2-(methylsulfanyl)-5-[2-(triisopropylsilyl)ethynyl]pyrido[2,3-d]pyrimidine (620 mg, 1.58 mmol, 1.0 eq.), dimethylamine (214 mg, 4.75 mmol, 3.0 eq.) and N,N-diisopropyl-N-ethylamine (818 mg, 6.33 mmol, 4.0 eq.) in dioxane (6 mL) was heated in a sealed tube at 80° C. for 4 h. After cooling, the mixture was partitioned between dichloromethane (50 mL) and water (50 mL). The separated aqueous phase was further extracted with dichloromethane (1×50 mL) and combined organics washed with brine, dried (MgSO₄) and concentrated under reduced pressure. The residue was purified by flash column chromatography eluting with ethyl acetate (5%) in 40-60 petroleum ether to afford the title compound as a light yellow solid (464 mg, 73%).

(ES, m/z): [M+H]⁺=401.2.

### 2-methanesulfonyl-N,N-dimethyl-5-[2-(triisopropylsilyl)ethynyl]pyrido[2,3-d]pyrimidin-7-amine

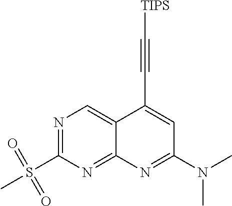

General Procedure 2 was applied to N,N-dimethyl-2-(methylsulfanyl)-5-[2-(triisopropylsilyl)ethynyl]pyrido[2,3-d]pyrimidin-7-amine (464 mg, 1.16 mmol) and m-CPBA (599 mg, 3.47 mmol) in dichloromethane (4.6 mL) to yield the title compound as a yellow solid (420 mg, 84%).

(ES, m/z): [M+H]⁺=433.2.

### N²-(4-{[2-(dimethylamino)ethyl](methyl)amino}phenyl)-N⁷,N⁷-dimethyl-5-[2-(triisopropylsilyl)ethynyl]pyrido[2,3-d]pyrimidine-2,7-diamine

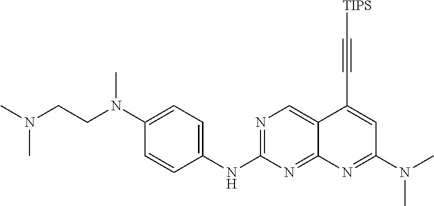

General Procedure 3 was applied to 2-methanesulfonyl-N,N-dimethyl-5-[2-(triisopropylsilyl)ethynyl]pyrido[2,3-d]pyrimidin-7-amine (420 mg, 0.971 mmol), N¹-[2-(dimethylamino)ethyl]-N¹-methylbenzene-1,4-diamine (225 mg, 1.16 mmol) and trifluoroacetic acid (221 mg, 1.94 mmol) in 2-butanol (4.2 mL). The solution mixture was stirred for 1 h at 100° C. The residue was purified by reverse phase flash column chromatography eluting with acetonitrile (30-60%) in water (0.1% formic acid) to afford the title compound as an orange solid (230 mg, 43%).

(ES, m/z): [M+H]⁺=546.4.

### N²-(4-{[2-(dimethylamino)ethyl](methyl)amino}phenyl)-5-ethynyl-N⁷,N⁷-dimethylpyrido[2,3-d]pyrimidine-2,7-diamine

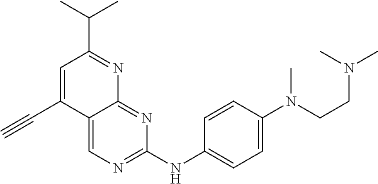 [SMILES: C#Cc1cc(C(C)C)nc2nc(Nc3ccc(N(C)CCN(C)C)cc3)ncc12]

General Procedure 1 was applied to N²-(4-{[2-(dimethylamino)ethyl](methyl)amino}phenyl)-N⁷,N⁷-dimethyl-5-[2-(triisopropylsilyl)ethynyl]pyrido [2,3-d]pyrimidine-2,7-diamine (250 mg, 0.458 mmol) and potassium fluoride (532 mg, 9.16 mmol) in THE (1 mL) and methanol (2 mL). The resulting mixture was stirred for 2 h at 80° C. The residue was purified by reverse phase flash column chromatography eluting with acetonitrile (30-60%) in water (0.1% formic acid) to yield the title compound as a yellow solid (106 mg, 60%).

(ES, m/z): [M+H]⁺=389.9

¹H NMR (400 MHz, DMSO-d6) δ 9.46 (s, 1H), 8.85 (s, 1H), 7.64 (d, J=8.4 Hz, 2H), 6.93 (s, 1H), 6.71 (d, J=8.5 Hz, 2H), 4.87 (s, 1H), 3.42 (t, J=7.1 Hz, 2H), 3.19 (s, 6H), 2.87 (s, 3H), 2.50 (t, J=7.1 Hz, 2H), 2.29 (s, 6H).

### Example 25

### 2-(methylsulfanyl)-N-phenyl-5-[2-(triisopropylsilyl)ethynyl]pyrido[2,3-d]pyrimidin-7-amine

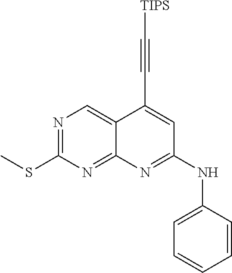

A solution of 7-chloro-2-(methylsulfanyl)-5-[2-(triisopropylsilyl)ethynyl]pyrido[2,3-d]pyrimidine (1.00 g, 2.55 mmol, 1.0 eq.), aniline (285 mg, 3.06 mmol, 1.2 eq.), XantPhos (147 mg, 0.255 mmol, 0.1 eq.), tris(dibenzyldeneacetone)dipalladium(0) (117 mg, 0.128 mmol, 0.05 eq.) and cesium carbonate (1.66 g, 5.10 mmol, 2.0 eq.) in dioxane (10 mL) was stirred at 110° C. The reaction mixture was diluted with ethyl acetate (50 mL) and water (50 mL). The ethyl acetate layer was separated and the aqueous layer was extracted with ethyl acetate (2×30 mL). The combined organic layers were dried (Na₂SO₄) and concentrated under reduced pressure. The residue was purified by flash column chromatography eluting with ethyl acetate (0-50%) in 40-60 petroleum ether to yield the title compound as an orange solid (600 mg, 46%).

(ES, m/z): [M+H]⁺=449.3

### 2-methanesulfonyl-N-phenyl-5-[2-(triisopropylsilyl)ethynyl]pyrido[2,3-d]pyrimidin-7-amine

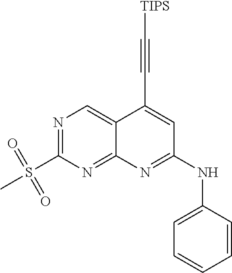

General Procedure 2 was applied to 2-(methylsulfanyl)-N-phenyl-5-[2-(triisopropylsilyl) ethynyl]pyrido[2,3-d]pyrimidin-7-amine (500 mg, 1.11 mmol) and m-CPBA (423 mg, 2.45 mmol) in dichloromethane (4 mL) to yield the title compound as an orange solid (300 mg, 50%), which was used without further purification.

(ES, m/z): [M+H]⁺=481.3

### N²-(4-{[2-(dimethylamino)ethyl](methyl)amino}phenyl)-N⁷-phenyl-5-[2-(triisoprooylsilylI)ethynyl]pyrido[2,3-d]pyrimidine-2.7-diamine

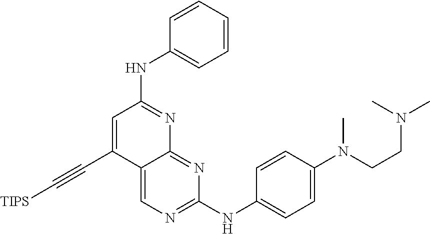

General Procedure 3 was applied to 2-methanesulfonyl-N-phenyl-5-[2-(triisopropylsilyl) ethynyl]pyrido[2,3-d]pyrimidin-7-amine (250 mg, 0.520 mmol), N¹-[2-(dimethylamino)ethyl]-N¹-methylbenzene-1,4-diamine (151 mg, 0.780 mmol) and trifluoroacetic acid (148 mg, 1.30 mmol) in 2-butanol (5 mL).The reaction mixture was stirred at 110° C. for 3 h. The crude material was purified by reverse phase flash column chromatography eluting with acetonitrile (5-100%) in water (0.1% trifluoroacetic acid) to yield the title compound as a red solid (120 mg, 32%).

(ES, m/z): [M+H]⁺=594.4

### N²-(4-{[2-(dimethylamino)ethyl](methyl)amino}phenyl)-5-ethynyl-N⁷-phenylpyrido[2,3-d]pyrimidine-2,7-diamine

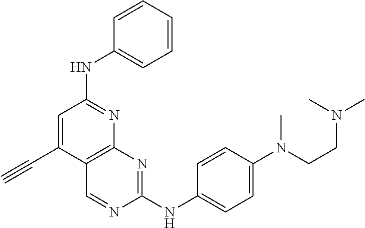 [SMILES: C#Cc1cc(Nc2ccccc2)nc2nc(Nc3ccc(N(C)CCN(C)C)cc3)ncc12]

General Procedure 1 was applied to N²-(4-{[2-(dimethylamino)ethyl](methyl)amino}phenyl)-N⁷-phenyl-5-[2-(triisopropylsilyl)ethynyl]pyrido[2,3-d]pyrimidine-2,7-diamine (120 mg, 0.202 mmol), potassium fluoride (117 mg, 2.02 mmol), THF (1.2 mL), methanol (0.6 mL) and water (0.1 mL).

The reaction mixture was stirred at room temperature for 3 h. The crude material was purified by reverse phase flash column chromatography eluting with acetonitrile (10-100%) in water (0.1% formic acid). The desired fractions were collected and concentrated under reduced pressure to remove most of the acetonitrile, then lyophilizied to dryness to yield the title compound as an orange solid (22 mg, 23%).

(ES, m/z): [M+H]⁺=483.0

¹H NMR (400 MHz, DMSO-d₆) δ 9.87 (s, 1H), 9.66 (s, 1H), 8.92 (s, 1H), 7.90 (d, J=8.0 Hz, 2H), 7.63 (s, 2H), 7.38 (t, J=7.8 Hz, 2H), 7.06 (t, J=7.4 Hz, 1H), 6.89 (s, 1H), 6.72 (d, J=8.7 Hz, 2H), 4.93 (s, 1H), 3.44 (t, J=7.2 Hz, 2H), 2.88 (s, 3H), 2.60 (s, 2H), 2.36 (s, 6H).

### Example 26

### 1-methyl-N-[2-(methylsulfanyl)-5-[2-(triisopropylsilyl)ethynyl]pyrido[2,3-d]pyrimidin-7-yl]imidazol-2-amine

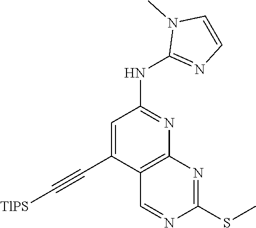

A solution of 7-chloro-2-(methylsulfanyl)-5-[2-(triisopropylsilyl)ethynyl]pyrido[2,3-d]pyrimidine (1.00 g, 2.55 mmol, 1.0 eq.), 1-methylmidazol-2-amine (300 mg, 3.06 mmol, 1.2 eq.), tris(dibenzyldeneacetone)dipalladium(0) (120 mg, 0.128 mmol, 0.05 eq.), XantPhos (150 mg, 0.255 mmol, 0.1 eq.) and cesium carbonate (1.66 g, 5.10 mmol, 2.0 eq.) in dioxane (15 mL) was stirred for 2 h at 100° C. The resulting mixture was diluted with water (100 mL) and extracted with ethyl acetate (50 mL). The residue was purified by flash column chromatography eluting with ethyl acetate (20%) in 40-60 petroleum ether to afford the title compound as a red solid (856 mg, 74%).

### N-{2-methanesulfonyl-5-[2-(triisopropylsilyl)ethynyl]pyrido[2,3-d]pyrimidin-7-yl}-1-methylmidazol-2-amine

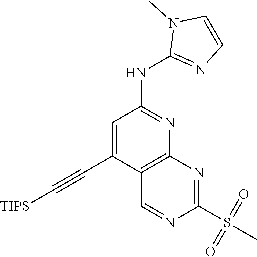

General Procedure 2 was applied to 1-methyl-N-[2-(methylsulfanyl)-5-[2-(triisopropylsilyl)ethynyl]pyrido[2,3-d]pyrimidin-7-yl]imidazol-2-amine (1.00 g, 2.21 mmol) and m-CPBA (1.15 g, 4.42 mmol) in dichloromethane (10 mL). The residue was purified by flash column chromatography eluting with ethyl acetate (50%) in 40-60 petroleum ether to afford the title compound as a red solid (760 mg, 71%).

### N⁷-(1-methylmidazol-2-yl)-N²-[4-(4-methylpiperazin-1-yl)phenyl]-5-[2-(triisopropylsilyl) ethynyl]pyrido[2,3-d]pyrimidine-2,7-diamine

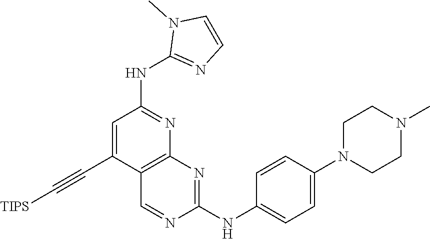

General Procedure 3 was applied to N-{2-methanesulfonyl-5-[2-(triisopropylsilyl)ethynyl]pyrido[2,3-d]pyrimidin-7-yl}-1-methylmidazol-2-amine (400 mg, 0.825 mmol), 4-(4-methylpiperazin-1-yl)aniline (237 mg, 1.24 mmol) and trifluoroacetic acid (0.12 mL, 1.65 mmol) in 2-butanol (5 mL). The reaction was stirred for 1 h at 100° C. The residue was purified by reverse phase flash column chromatography eluting with acetonitrile (10-50%) in water (0.1% formic acid) to yield the title compound as a red solid (150 mg, 31%).

### 5-ethynyl-N⁷-(1-methylmidazol-2-yl)-N²-[4-(4-methylpiperazin-1-yl)phenyl]pyrido[2,3-d]pyrimidine-2,7-diamine

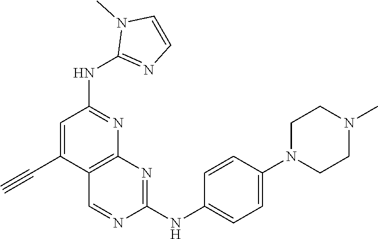 [SMILES: C#Cc1cc(Nc2nccn2C)nc2nc(Nc3ccc(N4CCN(C)CC4)cc3)ncc12]

General Procedure 1 was applied to N⁷-(1-methylmidazol-2-yl)-N²-[4-(4-methylpiperazin-1-yl)phenyl]-5-[2-(triisopropylsilyl)ethynyl]pyrido[2,3-d]pyrimidine-2,7-diamine (120 mg, 0.201 mmol) and potassium fluoride (117 mg, 2.01 mmol) in THE (2 mL), DMF (2 mL) and water (0.5 mL). The reaction was stirred for 1 h at 60° C. The residue was purified by reverse phase flash column chromatography eluting with acetonitrile (10-80%) in water (0.1% formic acid) to afford the title compound as an orange solid (56 mg, 63%).

(ES, m/z): [M+H]⁺=440.

¹H NMR (400 MHz, DMSO-d₆) δ 9.87 (s, 1H), 8.66 (s, 1H), 7.60 (d, J=8.4 Hz, 2H), 7.10 (s, 1H), 7.01-6.87 (m, 3H), 6.71 (s, 1H), 4.93 (s, 1H), 3.59 (s, 4H), 3.11 (t, J=5.0 Hz, 5H), 2.54 (t, J=4.0 Hz, 3H), 2.28 (s, 4H).

### Example 27

### 1-cyclopentyl-2-nitroimidazole

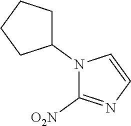 [SMILES: O=[N+]([O-])c1nccn1C1CCCC1]

A solution of 2-nitroimidazole (3.00 g, 26.5 mmol, 1.0 eq.) in DMF (30 mL) was treated with bromocyclopentane (5.93 g, 39.8 mmol, 1.5 eq.) for 10 minutes at room temperature followed by the addition of potassium carbonate (11.0 g, 79.6 mmol, 3.0 eq.) in portions at room temperature. The resulting mixture was stirred for 4 h at 70° C. The resulting mixture was extracted with ethyl acetate (2×30 mL). The combined organic layers were washed with brine (3×50 mL), dried (Na₂SO₄) and concentrated under reduced pressure to yield the title compound as a white solid (3.30 g, 68%).

### 1-cyclopentylmidazol-2-amine

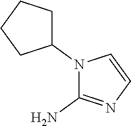 [SMILES: Nc1nccn1C1CCCC1]

To a solution of 1-cyclopentyl-2-nitroimidazole (1.50 g, 8.28 mmol, 1.0 eq.) in methanol (15 mL) and THF (15 mL) was added palladium on carbon (0.45 g, 4.23 mmol, 0.5 eq.). The mixture was hydrogenated at room temperature for 6 h under hydrogen atmosphere, filtered through a Celite pad washing with methanol (2×5 mL) and concentrated under reduced pressure to yield the title compound as a black solid (1.20 g, 96%). 1-cyclopentyl-N-[2-(methylsulfanyl)-5-[2-(triisopropylsilyl)ethynyl]pyrido[2,3-d]pyrimidin-7-yl]imidazol-2-amine

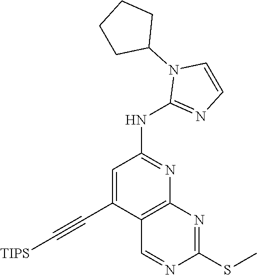

A solution of 7-chloro-2-(methylsulfanyl)-5-[2-(triisopropylsilyl)ethynyl]pyrido[2,3-d]pyrimidine (1.00 g, 2.55 mmol, 1.0 eq.), 1-cyclopentylmidazol-2-amine (500 mg, 3.32 mmol, 1.3 eq.), tris(dibenzyldeneacetone)dipalladium(0) (120 mg, 0.128 mmol, 0.05 eq.), XantPhos (150 mg, 0.255 mmol, 0.1 eq.) and cesium carbonate (1.66 g, 5.10 mmol, 2.0 eq.) in dioxane (10 mL) was stirred for 3 h at 80° C. The mixture was cooled to room temperature diluted with water (50 mL) and extracted with ethyl acetate (2×100 mL). The combined organic layers were concentrated under reduced pressure. The residue was purified by flash column chromatography eluting with ethyl acetate (20%) in 40-60 petroleum ether to afford the title compound as a red solid (812 mg, 63%).

### 1-cyclopentyl-N-{2-methanesulfonyl-5-[2-(triisopropylsilyl)ethynyl]pyrido[2,3-d]pyrimidin-7-yl}imidazol-2-amine

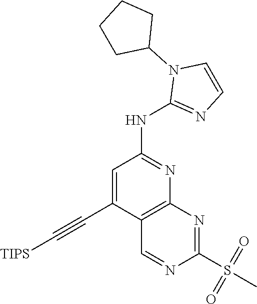

General Procedure 2 was applied to 1-cyclopentyl-N-[2-(methylsulfanyl)-5-[2-(triisopropylsilyl)ethynyl]pyrido[2,3-d]pyrimidin-7-yl]imidazol-2-amine (821 mg, 1.62 mmol) and m-CPBA (279 mg, 1.62 mmol) in dichloromethane (10 mL) to yield the title compound as a red solid (637 mg, 73%).

### N⁷-(1-cyclopentylmidazol-2-yl)-N²-[4-(4-methylpiperazin-1-yl)phenyl]-5-[2-(triisopropylsilylI)ethynyl]pyrido[2,3-d]pyrimidine-2.7-diamine

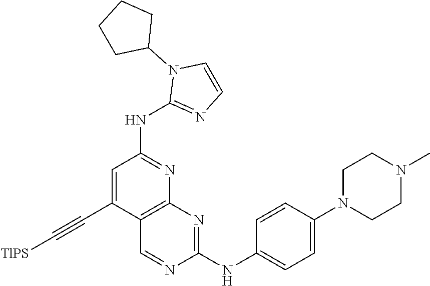

General Procedure 3 was applied to 1-cyclopentyl-N-{2-methanesulfonyl-5-[2-(triisopropylsilyl)ethynyl]pyrido[2,3-d]pyrimidin-7-yl}imidazol-2-amine (554 mg, 1.03 mmol), 4-(4-methylpiperazin-1-yl)aniline (295 mg, 1.54 mmol) and trifluoroacetic acid (0.150 mL, 2.06 mmol) in 2-butanol (5 mL). The reaction was stirred for 1 h at 100° C. The residue was purified by reverse phase flash column chromatography eluting with acetonitrile (10-50%) in water (0.1% formic acid) to yield the title compound as a red solid (408 mg, 61%).

### N⁷-(1-cyclopentylmidazol-2-yl)-5-ethynyl-N²-[4-(4-methylpiperazin-1-yl)phenyl]pyrido[2,3-d]pyrimidine-2,7-diamine

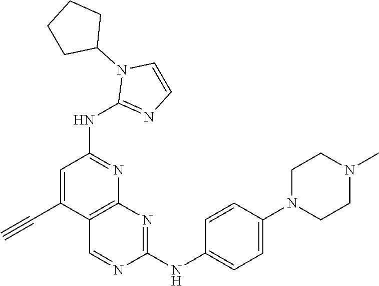 [SMILES: C#Cc1cc(Nc2nccn2C2CCCC2)nc2nc(Nc3ccc(N4CCN(C)CC4)cc3)ncc12]

General Procedure 1 was applied to N⁷-(1-cyclopentylmidazol-2-yl)-N²-[4-(4-methylpiperazin-1-yl)phenyl]-5-[2-(triisopropylsilyl)ethynyl]pyrido[2,3-d]pyrimidine-2,7-diamine (180 mg, 0.277 mmol) and potassium fluoride (161 mg, 2.77 mmol) in THF (2 mL), DMF (2 mL) and water (0.5 mL). The reaction was stirred for 1 h at 60° C. The residue was purified by reverse phase flash column chromatography eluting with acetonitrile (10-60%) in water (0.1% formic acid) to yield the title compound as a red solid (86.0 mg, 63%).

(ES, m/z): [M+H]⁺=494

¹H NMR (400 MHz, DMSO-d₆) δ 9.87 (s, 1H), 8.64 (d, J=9.5 Hz, 1H), 8.17 (s, 1H), 7.59 (d, J=8.4 Hz, 2H), 7.20 (d, J=1.7 Hz, 1H), 7.00 (d, J=1.6 Hz, 1H), 6.96-6.88 (m, 2H), 6.71 (s, 1H), 4.92 (s, 2H), 3.11 (t, J=4.9 Hz, 4H), 2.52 (s, 4H), 2.26 (s, 3H), 2.12-1.98 (m, 2H), 1.89-1.78 (m, 2H), 1.77-1.59 (m, 4H).

### Example 28

### 2-(methylsulfanyl)-5-[2-(triisopropylsilylethynyl]pyrido[2,3-d]pyrimidin-7-amine

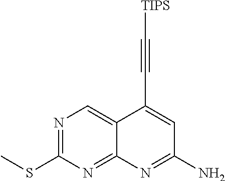

A solution of 7-chloro-2-(methylsulfanyl)-5-[2-(triisopropylsilyl)ethynyl]pyrido[2,3-d]pyrimidine (1.30 g, 3.32 mmol, 1.0 eq.), aqueous ammonia (6.50 mL, 229 mmol, 69 eq.) in isopropanol (13 mL, 0.25 M) was stirred for 2 h at 100° C. The mixture was cooled to room temperature and extracted with ethyl acetate (2×100 mL). The combined organic layers were washed with brine (2×100 mL), dried (Na₂SO₄) and concentrated under reduced pressure to yield the title compound as a yellow solid (820 mg, 66%).

(ES, m/z): [M+H]⁺=373.2

### N-[2-(methylsulfanyl)-5-[2-(triisopropylsilyl)ethynyl]pyrido[2,3-d]pyrimidin-7-yl]acetamide

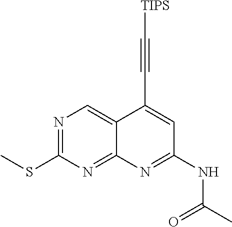

To a stirred solution of 2-(methylsulfanyl)-5-[2-(triisopropylsilyl)ethynyl]pyrido[2,3-d]pyrimidin-7-amine (820 mg, 2.20 mmol, 1.0 eq.) and triethylamine (445 mg, 4.40 mmol, 2,0 eq.) in dichloromethane (8 mL) was added acetyl chloride (345 mg, 4.40 mmol, 2.0 eq.) dropwise. The reaction was stirred at room temperature for 1 h then extracted with dichloromethane (2×100 mL). The combined organic layers were washed with brine (2×100 mL), dried (Na₂SO₄) and concentrated under reduced pressure to yield the title compound as a brown solid (800 mg, 88%).

(ES, m/z): [M+H]⁺=415.2

### N-{2-methanesulfonyl-5-[2-(triisopropylsilyl)ethynyl]pyrido[2,3-d]pyrimidin-7-yl}acetamide

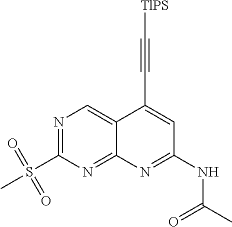

General Procedure 2 was applied to N-[2-(methylsulfanyl)-5-[2-(triisopropylsilyl)ethynyl]pyrido[2,3-d]pyrimidin-7-yl]acetamide (800 mg, 1.93 mmol) and m-CPBA (999 mg, 5.79 mmol) in dichloromethane (8 mL) The residue was purified by flash column chromatography eluting with ethyl acetate (10%) in 40-60 petroleum ether to afford the title compound as a yellow solid (250 mg, 29%).

(ES, m/z): [M+H]⁺=447.2

### N-{2-[(4-{[2-(dimethylamino)ethyl](methyl)amino]phenyl)amino}-5-[2-(triisopropylsilyl)ethynyl]pyrido[2,3-d]pyrimidin-7-yl}acetamide

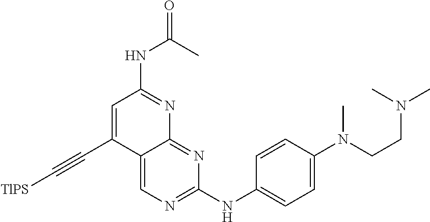

General Procedure 3 was applied to N-{2-methanesulfonyl-5-[2-(triisopropylsilyl)ethynyl]pyrido[2,3-d]pyrimidin-7-yl}acetamide (250 mg, 0.560 mmol), N¹-[2-(dimethylamino)ethyl]-N¹-methylbenzene-1,4-diamine (130 mg, 0.672 mmol) and trifluoroacetic acid (128 mg, 1.12 mmol) in 2-butanol (3 mL). The solution was stirred for 4 h at 60° C. The residue was purified by reverse phase flash column chromatography eluting with acetonitrile (30-60%) in water (0.1% formic acid) to yield the title compound as a yellow solid (180 mg, 57%).

(ES, m/z): [M+H]⁺=560.4

### N-{2-[(4-{[2-(dimethylamino)ethyl](methyl)amino}phenyl)amino]-5-ethynyl)pyrido[2,3-d]pyrimidin-7-yl}acetamide

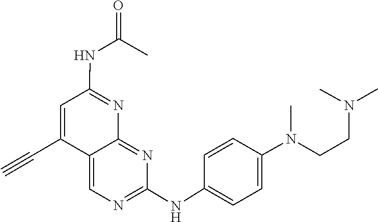 [SMILES: C#Cc1cc(NC(C)=O)nc2nc(Nc3ccc(N(C)CCN(C)C)cc3)ncc12]

General Procedure 1 was applied to N-{2-[(4-{[2-(dimethylamino)ethyl](methyl)amino}phenyl)amino]-5-[2-(triisopropylsilyl)ethynyl]pyrido[2,3-d]pyrimidin-7-yl}acetamide (180 mg, 0.322 mmol) and potassium fluoride (187 mg, 3.22 mmol) in THF (2 mL), methanol (2 mL) and water (50 uL). The resulting mixture was stirred for 2 h at room temperature. The residue was purified by reverse phase flash column chromatography eluting with acetonitrile (30-60%) in water (0.1% formic acid) to yield the title compound as a yellow solid (26.0 mg, 20%).

(ES, m/z): [M+H]⁺=404.2

¹H NMR (400 MHz, DMSO-d₆) δ 11.27 (s, 1H), 9.92 (s, 1H), 9.15 (s, 1H), 8.14 (s, 1H), 7.76 (s, 2H), 6.71 (d, J=2.2 Hz, 1H), 6.69 (d, J=2.3 Hz, 1H), 5.07 (s, 1H), 3.43 (t, J=7.2 Hz, 2H), 2.89 (s, 3H), 2.47 (d, J=7.1 Hz, 2H), 2.27 (s, 6H), 2.15 (s, 3H).

### Example 29

### N-[2-(methylsulfanyl)-5-[2-(triisopropylsilyl)ethynyl]pyrido[2,3-d]pyrimidin-7-yl]benzamide

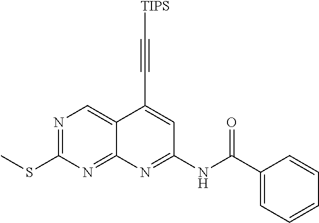

To a stirred solution of 2-(methylsulfanyl)-5-[2-(triisopropylsilyl)ethynyl]pyrido[2,3-d]pyrimidin-7-amine (1.00 g, 2.68 mmol, 1.0 eq.) in dichloromethane (10 mL, 0.2M) was added triethylamine (410 mg, 4.03 mmol, 1.5 eq.). The mixture was stirred for 5 minutes, then benzoyl chloride (570 mg, 4.03 mmol, 1.5 equiv) was added portionwise and the mixture was stirred for 2 h at room temperature. The reaction was quenched with water and extracted with ethyl acetate (3×20 mL). The combined organic layers were washed with brine (2×30 mL), dried (Na₂SO₄) and concentrated under reduced pressure. The residue was purified by flash column chromatography, eluting with ethyl acetate (20% in 40-60 petroleum ether to afford the title compound as a yellow solid (800 mg, 61%).

(ES, m/z): [M+H]⁺=477

### N-{2-methanesulfonyl-5-[2-(triisopropylsilyl)ethynyl]pyrido[2,3-d]pyrimidin-7-yl}benzamide

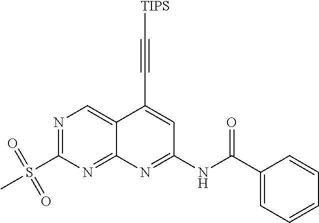

General Procedure 2 was applied to N-[2-(methylsulfanyl)-5-[2-(triisopropylsilyl)ethynyl]pyrido[2,3-d]pyrimidin-7-yl]benzamide (800 mg, 1.68 mmol) and m-CPBA (637 mg, 3.69 mmol) in dichloromethane (8 mL) to afford the title compound as a yellow solid (700 mg, 100%), which was used without further purification.

(ES, m/z): [M+H]⁺=509

### N-(2-{[4-(4-methylpiperazin-1-yl)phenyl]amino}-5-[2-(triisopropylsilyl)ethynyl]pyrido[2,3-d]pyrimidin-7-yl)benzamide

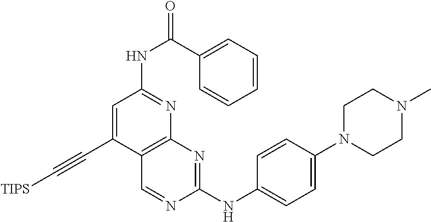

General Procedure 3 was applied to N-{2-methanesulfonyl-5-[2-(triisopropylsilyl)ethynyl]pyrido[2,3-d]pyrimidin-7-yl}benzamide (700 mg, 1.38 mmol), 4-(4-methylpiperazin-1-yl)aniline (395 mg, 2.06 mmol) trifluoroacetic acid (314 mg, 2.75 mmol) in 2-butanol (7 mL). The resulting mixture was stirred for 2 h at 100° C. The resulting mixture was purified by reverse phase flash column chromatography eluting with acetonitrile in water (0.4% formic acid) to afford the title compound as a yellow solid (300 mg, 34%).

(ES, m/z): [M+H]⁺=620

### N-(5-ethynyl-2-{[4-(4-methylpiperazin-1-yl)phenyl]amino}pyrido[2,3-d]pyrimidin-7-yl)benzamide

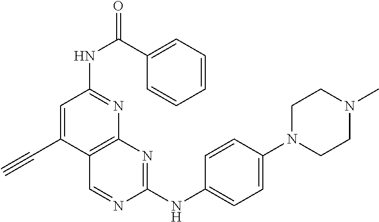 [SMILES: C#Cc1cc(NC(=O)c2ccccc2)nc2nc(Nc3ccc(N4CCN(C)CC4)cc3)ncc12]

General Procedure 1 was applied to N-(2-{[4-(4-methylpiperazin-1-yl)phenyl]amino}-5-[2-(triisopropylsilyl)ethynyl]pyrido[2,3-d]pyrimidin-7-yl)benzamide (300 mg, 0.484 mmol) and potassium fluoride (281 mg, 4.84 mmol) in DMF (3 mL) and THF (3 mL).

The resulting mixture was stirred for 1 h at 60° C. The residue was purified by reverse phase flash column chromatography eluting with acetonitrile in water (0.4% formic acid) to afford the title compound as an orange solid (87.5 mg, 37%).

(ES, m/z): [M+H]⁺=464 ¹H-NMR: (300 MHz, DMSO-d₆) δ 11.56 (s, 1H), 10.03 (s, 1H), 9.23 (s, 1H), 8.30 (s, 1H), 8.13-8.01 (m, 2H), 7.80 (d, J=8.5 Hz, 2H), 7.67-7.58 (m, 1H), 7.54 (dd, J=8.3, 6.7 Hz, 2H), 6.93 (d, J=9.1 Hz, 2H), 5.11 (s, 1H), 3.10 (t, J=5.0 Hz, 4H), 2.46 (d, J=5.2 Hz, 4H), 2.23 (s, 3H).

### Example 30

### N-[2-(methylsulfanyl)-5-[2-(triisopropylsilyl)ethynyl]pyrido[2,3-d]pyrimidin-7-yl]-2-phenylacetamide

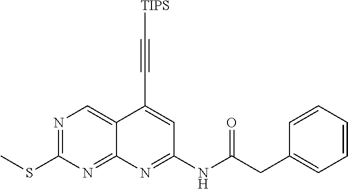

To a stirred solution of 2-(methylsulfanyl)-5-[2-(triisopropylsilyl)ethynyl]pyrido[2,3-d]pyrimidin-7-amine (100 mg, 0.268 mmol, 1.0 eq.) in dichloromethane (2 mL) was added triethylamine (54.3 mg, 0.536 mmol, 2.0 eq.). After 5 minutes stirring at room temperature, phenylacetyl chloride (83.0 mg, 0.536 mmol, 2.0 eq.) was added portionwise. The resulting mixture was stirred for 2 h at room temperature to yield the title compound.

(ES, m/z): [M+H]⁺=491

### N-{2-methanesulfonyl-5-[2-(triisopropylsilyl)ethynyl]pyrido[2,3-d]pyrimidin-7-yl}-2-phenylacetamide

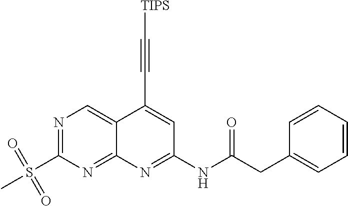

General Procedure 2 was applied to N-[2-(methylsulfanyl)-5-[2-(triisopropylsilyl)ethynyl]pyrido[2,3-d]pyrimidin-7-yl]-2-phenylacetamide (440 mg, 0.897 mmol) and m-CPBA (340 mg, 1.97 mmol) in dichloromethane (5 mL) to afford the title compound as a yellow solid (500 mg, 100%), which was used without further purification.

(ES, m/z): [M+H]⁺=523

### N-(2-{[4-(4-methylpiperazin-1-yl)phenyl]amino}-5-[2-(triisopropylsilyl)ethynyl]pyrido[2,3-d]pyrimidin-7-yl)-2-phenylacetamide

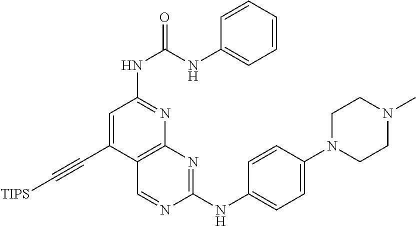

General Procedure 3 was applied to N-{2-methanesulfonyl-5-[2-(triisopropylsilyl)ethynyl]pyrido[2,3-d]pyrimidin-7-yl}-2-phenylacetamide (450 mg, 0.861 mmol), 4-(4-methylpiperazin-1-yl)aniline (246.99 mg, 1.29 mmol) and trifluoroacetic acid (196 mg, 1.72 mmol) in 2-butanol (5 mL). The resulting mixture was stirred for 2 hat 100° C.

The resulting mixture was purified by reverse phase flash column chromatography eluting with acetonitrile (40-65%) in water (0.4% formic acid) to afford the title compound as a yellow solid (100 mg, 18%).

(ES, m/z): [M+H]⁺=634

### N-(5-ethynyl-2-{[4-(4-methylpiperazin-1-yl)phenyl]amino}pyrido[2,3-d]pyrimidin-7-yl)-2-phenylacetamide

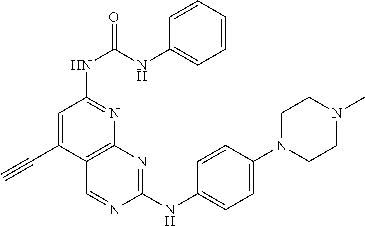 [SMILES: C#Cc1cc(NC(=O)Nc2ccccc2)nc2nc(Nc3ccc(N4CCN(C)CC4)cc3)ncc12]

General Procedure 1 was applied to N-(2-{[4-(4-methylpiperazin-1-yl)phenyl]amino}-5-[2-(triisopropylsilyl)ethynyl]pyrido[2,3-d]pyrimidin-7-yl)-2-phenylacetamide (100 mg, 0.158 mmol) and potassium fluoride (91.6 mg, 1.58 mmol) in DMF (2 mL) and THF (2 mL). The resulting mixture was stirred for 1 h at 60° C. The residue was purified by reverse phase flash column chromatography eluting with acetonitrile (10-50%) in water (0.4% formic acid) to afford the title compound as an orange solid (27.9 mg, 34%).

(ES, m/z): [M+H]⁺=478 ¹H-NMR: (300 MHz, DMSO-d₆) δ 11.58 (s, 1H), 10.05 (s, 1H), 9.18 (s, 1H), 8.15 (s, 1H), 7.84 (d, J=8.7 Hz, 2H), 7.40-7.22 (m, 5H), 6.94 (d, J=8.9 Hz, 2H), 5.08 (s, 1H), 3.79 (s, 2H), 3.10 (d, J=5.3 Hz, 4H), 2.47 (s, 4H), 2.24 (s, 3H).

### Example 31

### 1-[2-(methylsulfanyl)-5-[2-(triisopropylsilyl)ethynyl]pyrido[2,3-d]pyrimidin-7-yl]pyrrolidin-2-one

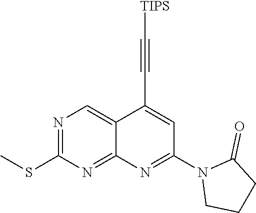

To a stirred mixture of 7-chloro-2-(methylsulfanyl)-5-[2-(triisopropylsilyl)ethynyl]pyrido[2,3-d]pyrimidine (1.00 g, 2.55 mmol, 1.0 eq.) and pyrrolidone (260 mg, 3.06 mmol, 1.2 eq.) in dioxane (10 mL) was added cesium carbonate (1.66 g, 5.10 mmol, 2.0 eq.), tris(dibenzyldeneacetone)dipalladium(0) (234 mg, 0.255 mmol, 0.1 eq.) and XantPhos (295 mg, 0.510 mmol, 0.2 eq.). The resulting mixture was stirred for additional 2 h at 80° C. The reaction was quenched with water (20 mL) and extracted with ethyl acetate (3×20 mL). The combined organic layers were washed with brine (2×30 mL), dried (Na₂SO₄) and concentrated under reduced pressure. The residue was purified by flash column chromatography eluting with ethyl acetate (20%) in 40-60 petroleum ether to afford the title compound as a yellow solid (629 mg, 54%).

### 1-{2-methanesulfonyl-5-[2-(triisopropylsilyl)ethynyl]pyrido [2,3-d]pyrimidin-7-yl}pyrrolidin-2-one

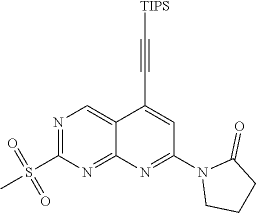

General Procedure 2 was applied to 1-[2-(methylsulfanyl)-5-[2-(triisopropylsilyl)ethynyl]pyrido[2,3-d]pyrimidin-7-yl]pyrrolidin-2-one (550 mg, 1.25 mmol) and m-CPBA (474 mg, 2.75 mmol) in dichloromethane (6 mL) to afford the title compound as a yellow solid (580 mg, 98%). The crude product was used without further purification. 1-(2-{[4-(4-methylpiperazin-1-yl)phenyl]amino}-5-[2-(triisopropylsilyl)ethynyl]pyrido[2,3-d]pyrimidin-7-yl)pyrrolidin-2-one

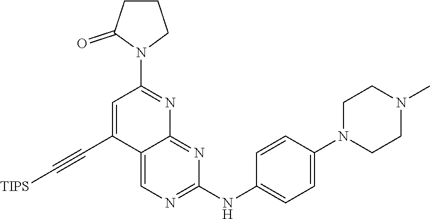

General Procedure 3 was applied to 1-{2-methanesulfonyl-5-[2-(triisopropylsilyl)ethynyl]pyrido[2,3-d]pyrimidin-7-yl}pyrrolidin-2-one (500 mg, 1.06 mmol), 4-(4-methylpiperazin-1-yl)aniline (242.80 mg, 1.27 mmol) and trifluoroacetic acid (241 mg, 2.12 mmol) in 2-butanol (5 mL). The resulting mixture was stirred for additional 2 h at 100° C. The mixture was purified by reverse phase flash column chromatography eluting with acetonitrile (40-60%) in water (0.4% formic acid) to afford the title compound as a yellow solid (300 mg, 47%).

### 1-(5-ethynyl-2-{[4-(4-methylpiperazin-1-yl)phenyl]amino}pyrido[2,3-d]pyrimidin-7-yl)pyrrolidin-2-one

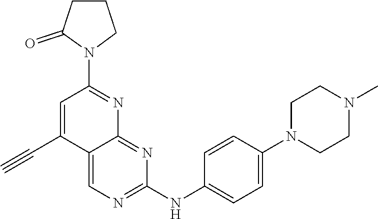 [SMILES: C#Cc1cc(N2CCCC2=O)nc2nc(Nc3ccc(N4CCN(C)CC4)cc3)ncc12]

General Procedure 1 was applied to 1-(2-{[4-(4-methylpiperazin-1-yl)phenyl]amino}-5-[2-(triisopropylsilyl)ethynyl]pyrido[2,3-d]pyrimidin-7-yl)pyrrolidin-2-one (200 mg, 0.343 mmol) and potassium fluoride (199 mg, 3.43 mmol) in THF (2 mL), DMF (2 mL) and water (0.5 mL). The resulting solution was stirred for 2 hours at 70° C. The reaction mixture was purified by reverse phase flash column chromatography eluting with acetonitrile (20-100%) in water (0.1% trifluoroacetic acid). The desired fractions were collected and concentrated under reduced pressure to remove most of the acetonitrile, then lyophilizied to afford the title compound as an orange solid (93.4 mg, 64%).

(ES, m/z): [M+H]⁺=431 ¹H-NMR: (300 MHz, DMSO-d₆, ppm): 5 9.98 (s, 1H), 9.20 (s, 1H), 8.39 (s, 1H), 7.73 (d, J=8.1 Hz, 2H), 6.95 (d, J=9 Hz, 2H), 5.07 (s, 1H), 4.13-4.10 (m, 2H), 3.12-3.09 (m, 4H), 2.69-2.64 (m, 2H), 2.51-2.50 (m, 4H), 2.23 (s, 3H), 2.10-2.07 (m, 2H).

### Example 32

### N-[2-(methylsulfanyl)-5-[2-(triisopropylsilyl)ethynyl]pyrido[2,3-d]pyrimidin-7-yl]-3-phenylpropanamide

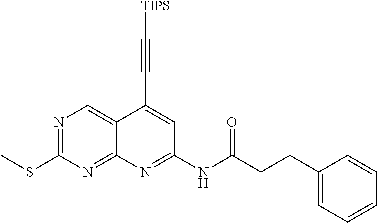

To a stirred solution of 2-(methylsulfanyl)-5-[2-(triisopropylsilyl)ethynyl]pyrido[2,3-d]pyrimidin-7-amine (1.00 g, 2.68 mmol, 1.0 eq.) in dichloromethane (10 mL) was added triethylamine (543 mg, 5.37 mmol, 2.0 eq.). After stirring for 5 minutes at room tempertaure benzenepropanoyl chloride (905 mg, 5.37 mmol, 2.0 eq.) was added portionwise. The resulting mixture was stirred for 2 h at room temperature then quenched with water and extracted with ethyl acetate (3×20 mL). The combined organic layers were washed with brine (2×30 mL), dried (Na₂SO₄) and concentrated under reduced pressure. The residue was purified by flash column chromatography eluting with ethyl acetate (20%) in 40-60 petroleum ether to afford the title compound as a yellow solid (650 mg, 47%).

(ES, m/z): [M+H]⁺=505

### N-{2-methanesulfonyl-5-[2-(triisopropylsilyl)ethynyl]pyrido[2,3-d]pyrimidin-7-yl}-3-Phenylpropanamide

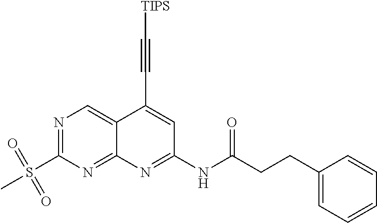

General Procedure 2 was applied to N-[2-(methylsulfanyl)-5-[2-(triisopropylsilyl)ethynyl]pyrido[2,3-d]pyrimidin-7-yl]-3-phenylpropanamide (550 mg, 1.09 mmol) and m-CPBA (414 mg, 2.40 mmol) in dichloromethane (11 mL) to afford the title compound as a yellow solid (650 mg, 100%). The crude product mixture was used without further purification.

(ES, m/z): [M+H]⁺=537

### N-(2-{[4-(4-methylpiperazin-1-yl)phenyl]amino}-5-[2-(triisopropylsilyl) ethynyl]pyrido[2,3-d]pyrimidin-7-yl)-3-phenylpropanamide

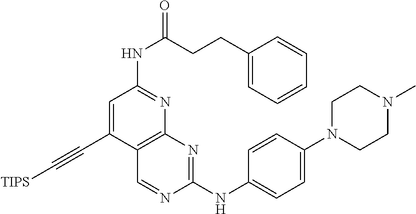

General Procedure 3 was applied to N-{2-methanesulfonyl-5-[2-(triisopropylsilyl)ethynyl]pyrido[2,3-d]pyrimidin-7-yl}-3-phenylpropanamide (550 mg, 1.03 mmol), 4-(4-methylpiperazin-1-yl)aniline (294 mg, 1.54 mmol) and trifluoroacetic acid (234 mg, 2.05 mmol) in 2-butanol (6 mL). The resulting mixture was stirred for 3 h at 60° C.

The resulting mixture was purified by reverse phase flash column chromatography eluting with acetonitrile (40-65%) in water (0.4% formic acid) to afford as a yellow solid (300 mg, 43%).

(ES, m/z): [M+H]⁺=648

### N-(5-ethynyl-2-{[4-(4-methylpiperazin-1-yl)phenyl]amino}pyrido[2,3-d]pyrimidin-7-yl)-3-phenylpropanamide

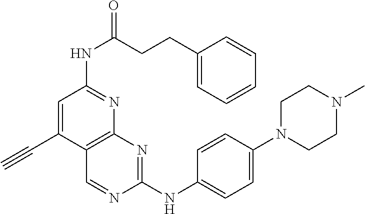 [SMILES: C#Cc1cc(NC(=O)CCc2ccccc2)nc2nc(Nc3ccc(N4CCN(C)CC4)cc3)ncc12]

General Procedure 1 was applied to N-(2-{[4-(4-methylpiperazin-1-yl)phenyl]amino}-5-[2-(triisopropylsilyl)ethynyl]pyrido[2,3-d]pyrimidin-7-yl)-3-phenylpropanamide (100 mg, 0.154 mmol) and potassium fluoride (89.6 mg, 1.54 mmol) in DMF (1 ml), THF (ml) and water (0.2 ml). The reaction was stirred for 1 h at 60° C.

The mixture was purified by reverse phase flash column chromatography eluting with acetonitrile (40-60%) in water (0.4% formic acid) to afford the title compound as an orange solid. (46.0 mg, 55%)

(ES, m/z): [M+H]⁺=492

¹H-NMR: (300 MHz, DMSO-d₆) δ 11.36 (s, 1H), 10.03 (s, 1H), 9.18 (d, J=6.8 Hz, 1H), 7.82 (d, J=8.6 Hz, 2H), 7.33-7.15 (m, 6H), 6.92 (d, J=8.8 Hz, 2H), 5.10 (d, J=5.6 Hz, 1H), 3.09 (t, J=5.0 Hz, 4H), 2.91 (d, J=7.7 Hz, 2H), 2.77 (t, J=7.7 Hz, 2H), 2.45 (d, J=5.3 Hz, 4H), 2.22 (s, 3H).

### Example 33

### N-[2-(methylsulfanyl)-5-[2-(triisopropylsilyl)ethynyl]pyrido[2,3-d]pyrimidin-7-yl]methanesulfonamide

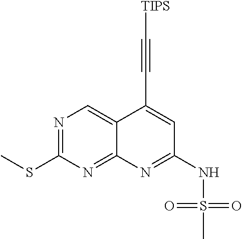

A solution of 2-(methylsulfanyl)-5-[2-(triisopropylsilyl)ethynyl]pyrido[2,3-d]pyrimidin-7-amine (1.60 g, 4.29 mmol, 1.0 eq.) and methane sulfonylchloride (590 mg, 5.15 mmol, 1.2 eq.) in pyridine (16 mL) was stirred for 96 h at room tempertaure. The resulting mixture was extracted with ethyl acetate (2×50 mL). The combined organic layers were washed with brine (2×50 mL), dried (Na₂SO₄) and concentrated under reduced pressure. The residue was purified by flash column chromatography eluting with ethyl acetate (13%) in 40-60 petroleum ether to afford the title compound as a light yellow solid (500 mg, 26%).

(ES, m/z): [M+H]⁺=451.2

### N-{2-methanesulfonyl-5-[2-(triisoprooylsilyl)ethynyl]pyrido[2,3-d]pyrimidin-7-yl}methanesulfonamide

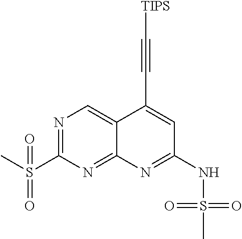

General Procedure 2 was applied to N-[2-(methylsulfanyl)-5-[2-(triisopropylsilyl)ethynyl]pyrido[2,3-d]pyrimidin-7-yl]methanesulfonamide (500 mg, 1.11 mmol) and m-CPBA (574 mg, 3.33 mmol) in dichloromethane (5 mL) to yield the title compound as a light yellow solid (500 mg, 93%).

(ES, m/z): [M+H]⁺=483.1

### N-{2-[(4-{[2-(dimethylamino)ethyl](methyl)amino}phenyl)amino]-5-[2-(triisopropylsilyl)ethynyl]pyrido[2,3-d]pyrimidin-7-yl}methanesulfonamide

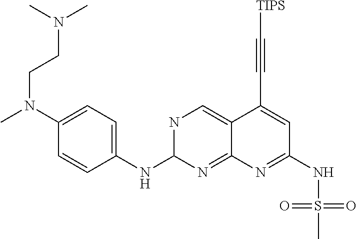

General Procedure 3 was applied to N-{2-methanesulfonyl-5-[2-(triisopropylsilyl)ethynyl]pyrido[2,3-d]pyrimidin-7-yl}methanesulfonamide (500 mg, 1.04 mmol), N¹-[2-(dimethylamino)ethyl]-N¹-methylbenzene-1,4-diamine (240 mg, 1.24 mmol) and trifluoroacetic acid (236 mg, 2.07 mmol) in 2-butanol (5 mL). The reaction mixture was stirred for 1 h at 100° C. The residue was purified by reverse phase flash column chromatography eluting with acetonitrile (30-60%) in water (0.1% formic acid) to yield the title compound as an orange solid (300 mg, 49%).

(ES, m/z): [M+H]⁺=596.3

### N-{2-[(4-{[2-(dimethylamino)ethyl](methyl)amino}phenyl)amino]-5-ethynyl)pyrido[2,3-d]pyrimidin-7-yl}methanesulfonamide

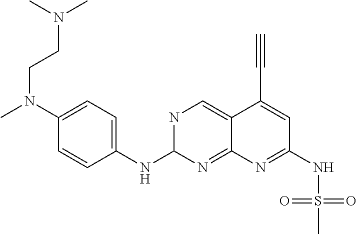 [SMILES: C#Cc1cc(NS(C)(=O)=O)nc2c1=CNC(Nc1ccc(N(C)CCN(C)C)cc1)N=2]

General Procedure 1 was applied to N-{2-[(4-{[2-(dimethylamino)ethyl](methyl)amino}phenyl)amino]-5-[2-(triisopropylsilyl)ethynyl]pyrido[2,3-d]pyrimidin-7-yl}methanesulfonamide (200 mg, 0.336 mmol) and potassium fluoride (390 mg, 6.72 mmol) in methanol (2 mL) and THF (2 mL). The resulting mixture was stirred for 2 h at 80° C. The residue was purified by reverse phase flash column chromatography eluting with acetonitrile (30-60%) in water (0.1% formic acid) to yield the title compound as a red solid (26.8 mg, 18%).

(ES, m/z): [M+H]⁺=439.9

¹H-NMR: 1H NMR (400 MHz, DMSO-d₆) δ 9.91 (s, 1H), 8.83 (s, 1H), 7.70 (s, 2H), 7.00 (s, 1H), 6.69 (d, J=9.0 Hz, 2H), 5.07 (s, 1H), 3.45 (t, J=7.2 Hz, 2H), 3.08 (s, 3H), 2.88 (s, 3H), 2.58 (t, J=7.3 Hz, 2H), 2.34 (s, 6H).

### Example 34

### 4-methyl-1-[2-(methylsulfanyl)-5-[2-(triisoproovisilyl)ethynyl]pyrido[2,3-d]pyrimidin-7-yl]pyrazole

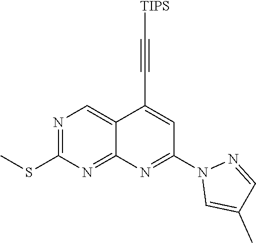

To a solution of 7-chloro-2-(methylsulfanyl)-5-[2-(triisopropylsilyl)ethynyl]pyrido[2,3-d]pyrimidine (800 mg, 2.04 mmol, 1.0 eq.) and 4-methyl-1H-pyrazole (201 mg, 2.45 mmol, 1.2 eq.) in dioxane (8 mL) were added cesium carbonate (1.33 mg, 4.08 mmol, 2.0 eq.), XantPhos (118 mg, 0.204 mmol, 0.1 eq.) and tris(dibenzyldeneacetone)dipalladium(0) (187 mg, 0.204 mmol, 0.1 eq.). After stirring for 2 h at 80° C., the mixture was concentrated under reduced pressure. The residue was purified by flash column chromatography eluting with ethyl acetate (5%) in 40-60 petroleum ether to afford the title compound as a yellow solid (700 mg, 78%).

(ES, m/z): [M+H]⁺=438.2

### 1-{2-methanesulfonyl-5-[2-(triisopropylsilyl)ethynyl]pyrido[2,3-d]pyrimidin-7-yl}-4-methylpyrazole

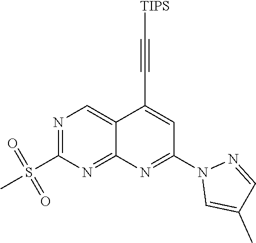

General Procedure 3 was applied to 4-methyl-1-[2-(methylsulfanyl)-5-[2-(triisopropylsilyl)ethynyl]pyrido[2,3-d]pyrimidin-7-yl]pyrazole (700 mg, 1.60 mmol) and m-CPBA (828 mg, 4.80 mmol) in dichloromethane (7 mL) to yield the title compound as a yellow solid (600 mg, 80%).

(ES, m/z): [M+H]⁺=470.2

### N¹-[2-(dimethylamino)ethyl]-N¹-methyl-N⁴-[7-(4-methylpyrazol-1-yl)-5-[2-(triisopropylsilyl)ethynyl]pyrido[2,3-d]pyrimidin-2-yl]benzene-1,4-diamine

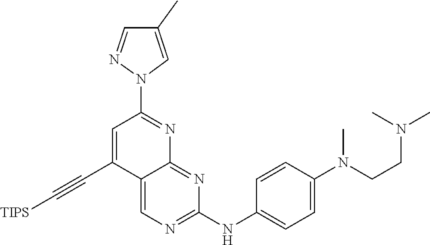

General Procedure 3 was applied to 1-{2-methanesulfonyl-5-[2-(triisopropylsilyl)ethynyl]pyrido[2,3-d]pyrimidin-7-yl}-4-methylpyrazole (600 mg, 1.28 mmol), N¹-[2-(dimethylamino)ethyl]-N¹-methyl benzene-1,4-diamine (296 mg, 1.53 mmol) and trifluoroacetic acid (291 mg, 2.55 mmol) in 2-butanol (6 mL). The reaction mixture was stirred for 2 h at 100° C. The residue was purified by reverse phase flash column chromatography eluting with acetonitrile (30-60%) in water (0.1% formic acid) to yield the title compound as a yellow solid (250 mg, 34%).

(ES, m/z): [M+H]⁺=583.4

### N¹-[2-(dimethylamino)ethyl]-N⁴-[5-ethynyl-7-(4-methylpyrazol-1-yl)pyrido[2,3-d]pyrimidin-2-yl]-N¹-methylbenzene-1,4-diamine

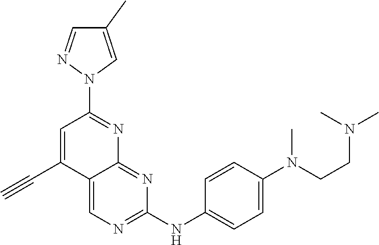 [SMILES: C#Cc1cc(-n2cc(C)cn2)nc2nc(Nc3ccc(N(C)CCN(C)C)cc3)ncc12]

General Procedure 1 was applied to N¹-[2-(dimethylamino)ethyl]-N¹-methyl-N⁴-[7-(4-methylpyrazol-1-yl)-5-[2-(triisopropylsilyl)ethynyl]pyrido[2,3-d]pyrimidin-2-yl]benzene-1,4-diamine (250 mg, 0.429 mmol) and potassium fluoride (249 mg, 4.29 mmol) in DMF (2 mL) and THF (2 mL). The resulting mixture was stirred for 2 h at 80° C. The residue was purified by reverse phase flash column chromatography eluting with acetonitrile (30-60%) in water (0.1% formic acid) to yield the title compound as a yellow solid (87.0 mg, 44%).

(ES, m/z): [M+H]⁺=427.20

¹H NMR (400 MHz, DMSO-d₆) δ 10.01 (s, 1H), 9.23 (s, 1H), 8.55 (s, 1H), 7.80 (d, J=12.7 Hz, 2H), 7.73 (s, 2H), 6.76-6.68 (m, 2H), 5.18 (s, 1H), 3.42 (q, J=9.3, 8.2 Hz, 2H), 2.90 (s, 3H), 2.44 (t, J=7.2 Hz, 2H), 2.23 (s, 6H), 2.16-2.12 (m, 3H).

### Example 35

### 1-[2-(methylsulfanyl)-5-[2-(triisopropylsilyl)ethynyl]pyrido [2,3-d]pyrimidin-7-yl]imidazole

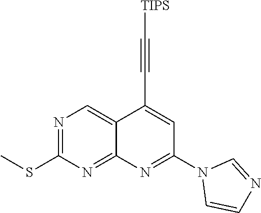

To a stirred mixture of 7-chloro-2-(methylsulfanyl)-5-[2-(triisopropylsilyl)ethynyl]pyrido[2,3-d]pyrimidine (700 mg, 1.79 mmol, 1.0 eq.) and pyrrole (144 mg, 2.14 mmol, 1.2 eq.) in dioxane (7 mL) was added cesium carbonate (1.16 g, 3.57 mmol, 2.0 eq.) and tris(dibenzyldeneacetone)dipalladium(0) (163 mg, 0.179 mmol, 0.1 eq.) and XantPhos (207 mg, 0.357 mmol, 0.2 eq.). The resulting mixture was stirred for 2 h at 80° C. then quenched with water and extracted with ethyl acetate (3×20 mL). The combined organic layers were washed with brine (2×50 mL), dried (Na₂SO₄) and concentrated under reduced pressure to afford the title compound as a yellow solid (300 mg, 39%).

(ES, m/z): [M+H]⁺=424

### 1-{2-methanesulfonyl-5-[2-(triisopropylsilyl)ethynyl]pyrido[2,3-d]pyrimidin-7-yl}imidazole

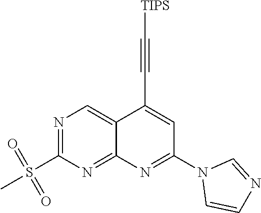

General Procedure 2 was applied to 1-[2-(methylsulfanyl)-5-[2-(triisopropylsilyl)ethynyl]pyrido[2,3-d]pyrimidin-7-yl]imidazole (300 mg, 0.708 mmol) and m-CPBA (269 mg, 1.56 mmol) in dichloromethane (5 mL) to afford the title compound as a yellow solid (400 mg, 100%), which was used in the next step without further purification.

(ES, m/z): [M+H]⁺=455

### 7-(imidazol-1-yl)-N-[4-(4-methylpiperazin-1-yl)phenyl]-5-[2-(triisopropylsilyl)ethynyl]pyrido[2,3-d]pyrimidin-2-amine

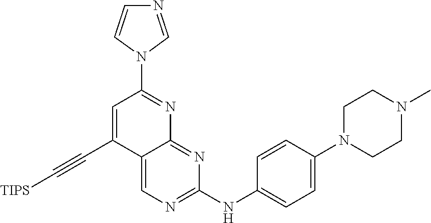

General Procedure 3 was applied to 1-{2-methanesulfonyl-5-[2-(triisopropylsilyl)ethynyl]pyrido[2,3-d]pyrimidin-7-yl}imidazole (400 mg, 0.878 mmol), 4-(4-methylpiperazin-1-yl)aniline (252 mg, 1.32 mmol) and trifluoroacetic acid (200 mg, 1.76 mmol) in 2-butanol (5 mL). The resulting mixture was stirred for 2 h at 100° C. The resulting mixture was purified by reverse phase flash column chromatography eluting with acetonitrile (40-65%) in water (0.4% formic acid) to afford the title compound as a red solid (120 mg, 24%).

(ES, m/z): [M+H]⁺=567

### 5-ethynyl-7-(imidazol-1-yl)-N-[4-(4-methylpiperazin-1-yl)phenyl]pyrido[2,3-d]pyrimidin-2-amine

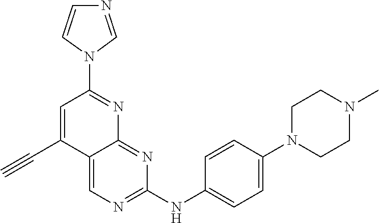 [SMILES: C#Cc1cc(-n2ccnc2)nc2nc(Nc3ccc(N4CCN(C)CC4)cc3)ncc12]

General Procedure 1 was applied to 7-(imidazol-1-yl)-N-[4-(4-methylpiperazin-1-yl)phenyl]-5-[2-(triisopropylsilyl)ethynyl]pyrido[2,3-d]pyrimidin-2-amine (120 mg, 0.212 mmol) and potassium fluoride (123 mg, 2.12 mmol) in DMF (2 mL) and THE (2 mL). The resulting mixture was stirred for 1 h at 60° C. The residue was purified by reverse phase flash column chromatography eluting with acetonitrile (10-50%) in water (0.4% formic acid) to afford the title compound as an orange solid (30.0 mg, 34%).

(ES, m/z): [M+H]⁺=411

1H-NMR: (300 MHz, DMSO-d₆) δ 9.48 (s, 1H), 9.37 (s, 1H), 8.33 (s, 1H), 7.98 (s, 1H), 7.77 (s, 2H), 7.58 (s, 1H), 7.02 (d, J=8.9 Hz, 2H), 5.13 (s, 1H), 3.77 (d, J=13.3 Hz, 2H), 3.50 (d, J=12.1 Hz, 2H), 3.16 (t, J=11.5 Hz, 2H), 2.93 (t, J=12.5 Hz, 2H), 2.84 (s, 3H).

### Example 36

### 1-[2-(methylsulfanyl)-5-[2-(triisopropylsilyl)ethynyl]pyrido[2,3-d]pyrimidin-7-yl]pyrrole

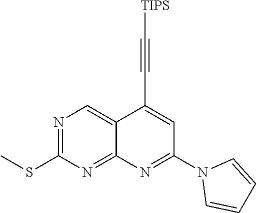

To a stirred mixture of 7-chloro-2-(methylsulfanyl)-5-[2-(triisopropylsilyl)ethynyl]pyrido[2,3-d]pyrimidine (700 mg, 1.79 mmol, 1.0 eq.) and pyrrole (144 mg, 2.14 mmol, 1.2 eq.) in 1,2-dioxane (10 mL) was added cesium carbonate (1163.55 mg, 3.572 mmol, 2 eq.) and tris(dibenzyldeneacetone)dipalladium(0) (164 mg, 0.179 mmol, 0.1 eq.) and XantPhos (207 mg, 0.357 mmol, 0.2 eq.). The final reaction mixture was stirred under microwave radiation for 2 h at 80° C. then quenched with water and extracted with ethyl acetate (3×20 mL). The combined organic layers were washed with brine (2×50 mL), dried (Na₂SO₄) and concentrated under reduced pressure to afford the title compound as a yellow solid (400 mg, 50%).

(ES, m/z): [M+H]⁺=423

### 1-{2-methanesulfonyl-5-[2-(triisopropylsilyl)ethynyl]pyrido[2,3-d]pyrimidin-7-yl}pyrrole

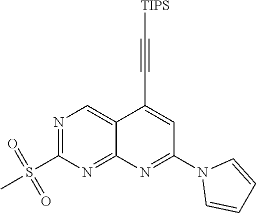

General Procedure 2 was applied to 1-[2-(methylsulfanyl)-5-[2-(triisopropylsilyl)ethynyl]pyrido[2,3-d]pyrimidin-7-yl]pyrrole (360 mg, 0.852 mmol) and m-CPBA (294 mg, 1.70 mmol) in dichloromethane (4 mL) to afford the title compound as a yellow solid, which was used in the next step without further purification.

(ES, m/z): [M+H]⁺=455

### N-[4-(4-methylpiperazin-1-yl)phenyl]-7-(pyrrol-1-yl)-5-[2-(triisopropylsilyl)ethynyl]pyrido[2,3-d]pyrimidin-2-amine

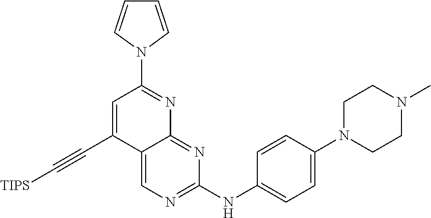

General Procedure 3 was applied to 1-{2-methanesulfonyl-5-[2-(triisopropylsilyl)ethynyl]pyrido[2,3-d]pyrimidin-7-yl}pyrrole (250 mg, 0.550 mmol), 4-(4-methylpiperazin-1-yl)aniline (158 mg, 0.825 mmol) and trifluoroacetic acid (125 mg, 1.10 mmol) in 2-butanol (5 mL). The resulting mixture was stirred for 2 h at 100° C. The resulting mixture was purified by reverse phase flash column chromatography eluting with acetonitrile (40-65%) in water (0.4% formic acid) to afford the title compound as a yellow solid (120 mg, 36%).

(ES, m/z): [M+H]⁺=566

### 5-ethynyl-N-[4-(4-methylpiperazin-1-yl)phenyl]-7-(pyrrol-1-yl)pyrido[2,3-d]pyrimidin-2-amine

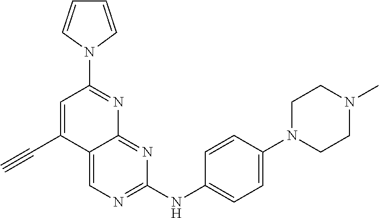 [SMILES: C#Cc1cc(-n2cccc2)nc2nc(Nc3ccc(N4CCN(C)CC4)cc3)ncc12]

General Procedure 1 was applied to N-[4-(4-methylpiperazin-1-yl)phenyl]-7-(pyrrol-1-yl)-5-[2-(triisopropylsilyl)ethynyl]pyrido[2,3-d]pyrimidin-2-amine (120 mg, 0.212 mmol) and potassium fluoride (123 mg, 2.12 mmol) in DMF (2 mL) and THF (2 mL). The resulting mixture was stirred for 1 h at 60° C. The residue was purified by reverse phase flash column chromatography eluting with acetonitrile (10-50%) in water (0.4% formic acid) to afford the title compound as an orange solid (31.0 mg, 33%).

(ES, m/z): [M+H]⁺=410

¹H-NMR: (300 MHz, DMSO-d₆) δ 10.06 (s, 1H), 9.24 (s, 1H), 7.93-7.87 (m, 3H), 7.76 (s, 2H), 7.01-6.91 (m, 2H), 6.42-6.36 (m, 2H), 5.19 (s, 1H), 3.11 (t, J=5.0 Hz, 4H), 2.47 (t, J=5.0 Hz, 4H), 2.23 (s, 3H).

### Example 37

### 3,3-dimethyl-1-[2-(methylsulfanyl)-5-[2-(triisopropylsilyl)ethynyl]pyrido[2,3-d]pyrimidin-7-yl]urea

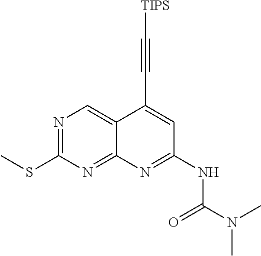

To a stirred mixture of 7-chloro-5-[2-(triisopropylsilyl)ethynyl]pyrido[2,3-d]pyrimidine-2-thiol (1.00 g, 2.65 mmol, 1.0 eq.) and 3,3-dimethylurea (280 mg, 3.17 mmol, 1.2 eq.) in dioxane (2 mL) was added cesium carbonate (1.72 mg, 5.29 mmol, 2.0 eq.), tris(dibenzyldeneacetone)dipalladium(0) (242 mg, 0.265 mmol, 0.1 eq.) and XantPhos (306 mg, 0.529 mmol, 0.2 eq.). The resulting mixture was stirred for 2 h at 80° C. then quenched with water (20 mL) and extracted with ethyl acetate (3×20 mL). The combined organic layers were washed with brine (2×30 mL), dried (Na₂SO₄) and concentrated under reduced pressure. The residue was purified by flash column chromatography eluting with ethyl acetate (16%) in 40-60 petroleum ether to afford the title compound as a yellow solid (450 mg, 38%).

### 1-{2-methanesulfonyl-5-[2-(triisopropylsilyl)ethynyl]pyrido[2,3-d]pyrimidin-7-yl}-3,3-dimethylurea

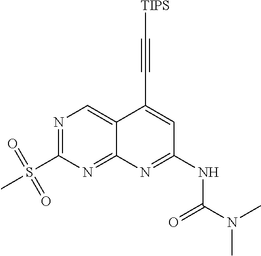

General Procedure 2 was applied to 3,3-dimethyl-1-[2-(methylsulfanyl)-5-[2-(triisopropylsilyl) ethynyl]pyrido[2,3-d]pyrimidin-7-yl]urea (400 mg, 0.902 mmol) and m-CPBA (389 mg, 2.25 mmol) in dichloromethane (4 mL) to yield the title compound as a yellow solid (400 mg, 93%).

### 3,3-dimethyl-1-(2-{[4-(4-methylpiperazin-1-yl)phenyl]amino}-5-[2-(triisopropylsilyl)ethynyl]pyrido[2,3-d]pyrimidin-7-yl)urea

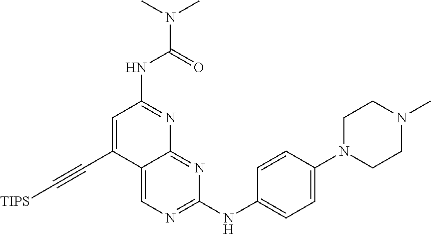

General Procedure 3 was applied to 1-{2-methanesulfonyl-5-[2-(triisopropylsilyl)ethynyl]pyrido[2,3-d]pyrimidin-7-yl}-3,3-dimethylurea (250 mg, 0.526 mmol), trifluoroacetic acid (120 mg, 1.05 mmol), 4-(4-methylpiperazin-1-yl)aniline (121 mg, 0.631 mmol) in 2-butanol (3 mL). The resulting solution was stirred for 3 h at 100° C. The crude material was purified by reverse phase flash column chromatography eluting with acetonitrile (70-100%) in water (0.1% trifluoroacetic acid) to yield the title compound as a red solid (200 mg, 65%).

### 1-(5-ethynyl-2-{[4-(4-methylpiperazin-1-yl)phenyl]amino}pyrido[2,3-d]pyrimidin-7-yl)-3,3-dimethylurea

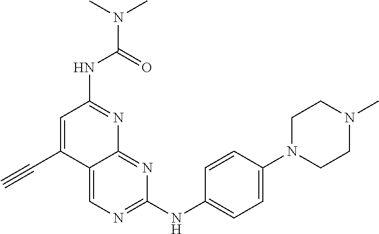 [SMILES: C#Cc1cc(NC(=O)N(C)C)nc2nc(Nc3ccc(N4CCN(C)CC4)cc3)ncc12]

General Procedure 1 was applied to 3,3-dimethyl-1-(2-{[4-(4-methylpiperazin-1-yl)phenyl]amino}-5-[2-(triisopropylsilyl)ethynyl]pyrido[2,3-d]pyrimidin-7-yl)urea (150 mg, 0.256 mmol) and potassium fluoride (148 mg, 2.56 mmol) in THF (2 mL), DMF (2 mL) and water (0.5 mL). The resulting solution was stirred for 2 h at 70° C. The reaction mixture was purified by reverse phase flash column chromatography eluting with acetonitrile (20-100%) in water (0.1% trifluoroacetic acid). The desired fractions were collected and concentrated under reduced pressure to remove most of the acetonitrile then lyophilizied to yield the title compound as a red solid (95.1 mg, 86%).

(ES, m/z): [M+H]⁺=431

¹H-NMR: (300 MHz, DMSO-d₆): δ 9.90 (s, 1H), 9.10 (s, 1H), 8.23 (s, 1H), 7.89 (s, 1H), 7.78 (d, J=8.4 Hz, 2H), 6.92 (d, J=9 Hz, 2H), 5.02 (s, 1H), 3.11-3.09 (m, 4H), 2.99 (s, 6H), 2.51-2.50 (m, 4H), 2.23 (s, 3H).

### Example 38

### 1-cyclopentyl-3-(2-(methylthio)-5-((triisopropylsilyl)ethynyl)pyrido[2,3-d]pyrimidin-7-yl)urea

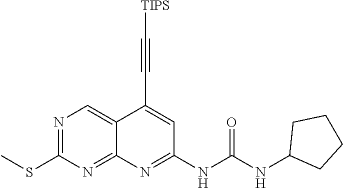

General Procedure 7 was applied to 2-(methylsulfanyl)-5-[2-(triisopropylsilyl)ethynyl]pyrido[2,3-d]pyrimidin-7-amine (230 mg, 0.610 mmol), sodium hydride (22.0 mg, 0.916 mmol), CDI (148 mg, 0.916 mmol) and cyclopentylamine (105 mg, 1.23 mmol) in DMF (3 mL). The residue was purified by flash column chromatography eluting with ethyl acetate (10%) in 40-60 petroleum ether to afford the title compound as a light yellow solid. (73.0 mg, 56%)

(ES, m/z): [M+H]⁺=484.4

¹H NMR (500 MHz, CDCl₃): δ_{H}=1.08 (3H, s), 1.09 (18H, s), 1.54-1.71 (4H, m), 1.74-1.84 (2H, m), 1.94-2.03 (2H, m), 2.59 (3H, s), 4.21 (1H, m), 7.24 (1H, s), 9.18 (1H, s), 10.6 (1H, s).

### 1-cyclopentyl-3-(2-((4-(4-methylpiperazin-1-yl)phenyl)amino)-5-((triisopropylsilyl)ethynyl)pyrido[2,3-d]pyrimidin-7-yl)urea

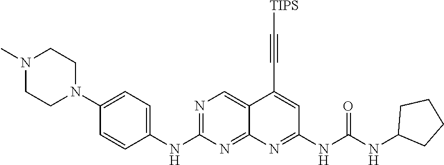

General Procedure 2 was applied to 1-cyclopentyl-3-[2-(methylsulfanyl)-5-[2-(triisopropylsilyl)ethynyl]pyrido[2,3-d]pyrimidin-7-yl]urea (73.0 mg, 0.150 mmol) and m-CPBA (78.0 mg, 0.453 mmol) in dichloromethane (3 mL) to yield the title compound (90.0 mg, 100%).

General Procedure 3 was applied to 1-cyclopentyl-3-{2-methanesulfonyl-5-[2-(triisopropylsilyl)ethynyl]pyrido[2,3-d]pyrimidin-7-yl}urea (90.0 mg, 0.170 mmol), 4-(4-methylpiperazin-1-yl)aniline (29.0 mg, 0.225 mmol) and trifluoroacetic acid (17.0 μL, 3.10 mmol) in acetonitrile (1.5 mL). The resulting mixture was stirred at 85° C. overnight. The residue was purified by flash column chromatography eluting with methanol (10%) in dichloromethane to yield the title compound as an orange red solid (54.0 mg, 47%).

(ES, m/z): [M+H]⁺=627.6

¹H NMR (500 MHz, CDCl₃): δ_{H}=1.16 (3H, s), 1.17 (18H, s), 1.65-1.78 (4H, m), 1.80-1.94 (2H, m), 1.97-2.08 (2H, m), 2.48 (3H, s), 2.78 (4H, s), 3.27 (4H, s), 4.30 (1H, s), 6.94 (2H, d, J=8.5 Hz), 7.28 (1H, s), 7.69 (2H, m), 9.17 (1H, s), 10.0 (1H, s).

### 1-cyclopentyl-3-(5-ethynyl-2-((4-(4-methylpiperazin-1-yl)phenyl)amino)pyrido[2,3-d]pyrimidin-7-yl)urea

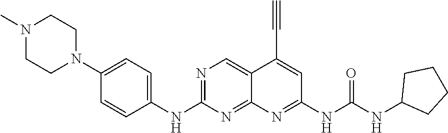 [SMILES: C#Cc1cc(NC(=O)NC2CCCC2)nc2nc(Nc3ccc(N4CCN(C)CC4)cc3)ncc12]

General Procedure 1 was applied to 1-cyclopentyl-3-(2-{[4-(4-methylpiperazin-1-yl)phenyl]amino}-5-[2-(triisopropylsilyl)ethynyl]pyrido[2,3-d]pyrimidin-7-yl)urea (340 mg, 0.540 mmol) and potassium fluoride (630 mg, 1.08 mmol) in DMF (9.4 mL). The reaction mixture was stirred at room temperature overnight. The residue was purified by flash column chromatography eluting with methanol (10%) in dichloromethane to yield the title compound as an orange solid (73.0 mg, 56%)

(ES, m/z): [M+H]⁺=471.4

¹H NMR (500 MHz, DMSO): δ_{H}=1.56 (2H, m), 1.70 (2H, m), 1.80 (2H, m), 1.88 (2H, m), 2.34 (3H, s), 2.56-2.70 (4H, s), 3.13 (4H, s), 4.15 (1H, sl), 5.05 (1H, s), 6.90 (2H, d, J=9.0 Hz), 7.16 (1H, s), 7.85 (2H, sl), 9.05 (1H, s), 9.98 (1H, sl), 10.0 (1H, s).

### Example 39

### 1-benzyl-3-[2-(methylsulfanyl)-5-[2-(triisopropylsilyl)ethynyl]pyrido[2,3-d]pyrimidin-7-yl]urea

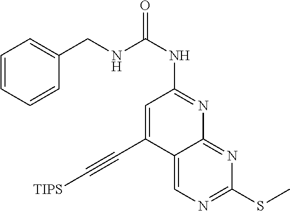

General Procedure 7 was applied to 2-(methylsulfanyl)-5-[2-(triisopropylsilyl)ethynyl]pyrido[2,3-d]pyrimidin-7-amine (800 mg, 2.15 mmol), triethylamine (447.66 mg, 4.42 mmol), CDI (522 mg, 3.22 mmol) and benzylamine (460 mg, 4.29 mmol) in dichloromethane (8 mL). The residue was purified by flash column chromatography eluting with ethyl acetate (12%) in 40-60 petroleum ether to afford the title compound as a light yellow solid. (700 mg, 64%)

(ES, m/z): [M+H]⁺=506.2

### 1-benzyl-3-{2-methanesulfonyl-5-[2-(triisopropylsilyl)ethynyl]pyrido[2,3-d]pyrimidin-7-yl}urea

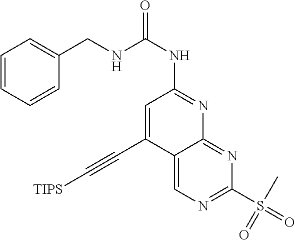

General Procedure 2 was applied to 1-benzyl-3-[2-(methylsulfanyl)-5-[2-(triisopropylsilyl)ethynyl]pyrido[2,3-d]pyrimidin-7-yl]urea (700 mg, 1.38 mmol) and m-CPBA (525 mg, 3.05 mmol) in dichloromethane (7 mL) to yield the title compound as a black oil (730 mg, 98%).

(ES, m/z): [M+H]⁺=538.2

### 1-benzyl-3-(2-{[4-(4-methylpiperazin-1-yl)phenyl]amino}-5-[2-(triisoprooylsilyl)ethynyl]pyrido[2,3-d]pyrimidin-7-yl)urea

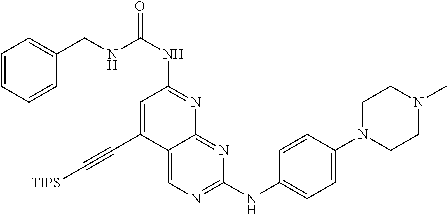

General Procedure 3 was applied to 1-benzyl-3-{2-methanesulfonyl-5-[2-(triisopropylsilyl)ethynyl]pyrido[2,3-d]pyrimidin-7-yl}urea (833 mg, 1.55 mmol), 4-(4-methylpiperazin-1-yl)aniline (444 mg, 2.32 mmol) and trifluoroacetic acid (353 mg, 3.10 mmol) in 2-butanol (8 mL). The resulting mixture was stirred for 2 h at 100° C. The residue was purified by reverse phase flash column chromatography eluting with methanol (10-50%) in water to yield the title compound as an orange red solid (430 mg, 43%).

(ES, m/z): [M+H]⁺=649.4

### 1-benzyl-3-(5-ethynyl-2-{[4-(4-methylpiperazin-1-yl)phenyl]amino}pyrido[2,3-d]pyrimidin-7-yl)urea

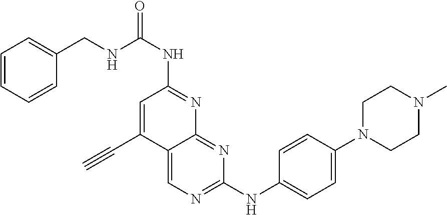 [SMILES: C#Cc1cc(NC(=O)NCc2ccccc2)nc2nc(Nc3ccc(N4CCN(C)CC4)cc3)ncc12]

General Procedure 1 was applied to 1-benzyl-3-(2-{[4-(4-methylpiperazin-1-yl)phenyl]amino}-5-[2-(triisopropylsilyl)ethynyl]pyrido[2,3-d]pyrimidin-7-yl)urea (400 mg, 0.616 mmol) and potassium fluoride (358 mg, 6.16 mmol) in THE (4 mL), DMF (4 mL) and water (400 uL). The reaction mixture was stirred for 2 h at room temperature. The residue was purified by reverse phase flash column chromatography eluting with acetonitrile (10-50%) in water to yield the title compound as an orange solid (50.0 mg, 16%).

(ES, m/z): [M+H]⁺=493.2

¹H NMR (400 MHz, DMSO-d₆) δ 10.22 (s, 1H), 9.93 (s, 1H), 9.29 (s, 1H), 9.06 (s, 1H), 7.71 (s, 2H), 7.45-7.22 (m, 6H), 6.84 (d, J=8.8 Hz, 2H), 5.05 (s, 1H), 4.50 (d, J=5.7 Hz, 2H), 3.07 (t, J=5.0 Hz, 4H), 2.47 (t, J=4.9 Hz, 4H), 2.23 (s, 3H), 1.23 (s, 1H).

### Example 40

### 3-(cyclopentylmethyl)-1-[2-(methylsulfanyl)-5-[2-(triisopropylsilyl)ethynyl]pyrido[2,3-D]pyrimidin-7-yl]urea

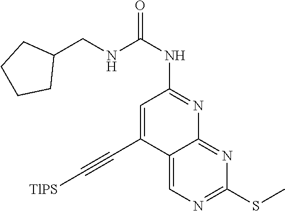

General Procedure 7 was applied to 2-(methylsulfanyl)-5-[2-(triisopropylsilyl)ethynyl]pyrido[2,3-d]pyrimidin-7-amine (300 mg, 0.81 mmol), triethylamine (167 uL, 1.20 mmol), CDI (261 mg, 1.20 mmol) and 1-cyclopentylmethanamine (160 mg, 1.61 mmol) in dichloromethane (3 mL) to yield the crude title compound as a yellow solid (598 mg, 149%).

(ES, m/z): [M+H]⁺=499

### 3-(cyclopentylmethyl)-1-{2-methanesulfonyl-5-[2-(triisopropylsilyl)ethynyl]pyrido[2,3-d]pyrimidin-7-yl}urea

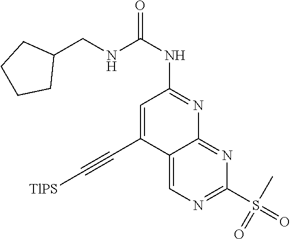

General Procedure 2 was applied to 3-(cyclopentylmethyl)-1-[2-(methylsulfanyl)-5-[2-(triisopropylsilyl)ethynyl]pyrido[2,3-d]pyrimidin-7-yl]urea (555 mg, 1.22 mmol) and m-CPBA (462 mg, 2.68 mmol) in dichloromethane (5.55 mL) to yield the crude title product as a brown solid (869 mg, 147%).

(ES, m/z): [M+H]⁺=530

### 3-(cyclopentylmethyl)-1-(2-{[4-(4-methylpiperazin-1-yl)phenyl]amino}-5-[2-(triisopropylsilyl)ethynyl]pyrido[2,3-d]pyrimidin-7-yl)urea

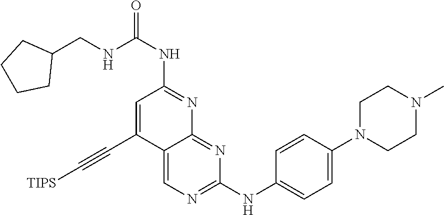

General Procedure 3 was applied to 3-(cyclopentylmethyl)-1-{2-methanesulfonyl-5-[2-(triisopropylsilyl)ethynyl]pyrido[2,3-d]pyrimidin-7-yl}urea (722 mg, 1.46 mmol), 4-(4-methylpiperazin-1-yl)aniline (418 mg, 2.19 mmol) and trifluoroacetic acid (217 uL, 2.92 mmol) in 2-butanol (7.7 mL). The resulting mixture was stirred for 1 h at 100° C. The residue was purified by reverse phase flash column chromatography eluting with acetonitrile (10-50%) in water (0.1% formic acid) to yield the title compound as a yellow solid (393 mg, 42%).

(ES, m/z): [M+H]⁺=642

### 3-(cyclopentylmethyl)-1-(5-ethynyl-2-{[4-(4-methylpiperazin-1-yl)phenyl]amino}pyrido[2,3-d]pyrimidin-7-yl)urea

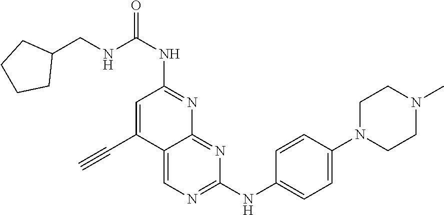 [SMILES: C#Cc1cc(NC(=O)NCC2CCCC2)nc2nc(Nc3ccc(N4CCN(C)CC4)cc3)ncc12]

General Procedure 1 was applied to 3-(cyclopentylmethyl)-1-(2-{[4-(4-methylpiperazin-1-yl)phenyl]amino}-5-[2-(triisopropylsilyl)ethynyl]pyrido[2,3-d]pyrimidin-7-yl)urea (250 mg, 0.390 mmol) and potassium fluoride (227 mg, 3.90 mmol) in THF (1.25 mL), DMF (1.25 mL) and water (100 uL). The resulting mixture was stirred for 1 h at 60° C. The residue was purified by reverse phase flash column chromatography eluting with acetonitrile (10-50%) in water (0.1% formic acid) to yield the title compound as a yellow solid (48.2 mg, 25%).

(ES, m/z): [M+H]⁺=485.

¹H NMR (400 MHz, DMSO-d₆) δ 10.07 (s, 1H), 9.95 (s, 1H), 9.34 (s, 1H), 9.05 (s, 1H), 7.86 (s, 3H), 7.15 (s, 2H), 6.90 (s, 2H), 3.24 (s, 2H), 3.08 (s, 3H), 2.46 (s, 4H), 2.22 (s, 3H), 2.06 (s, 2H), 1.82 (s, 2H), 1.63 (s, 2H), 1.54 (s, 2H).

### Example 41

### 3-cyclobutyl-1-[2-(methylsulfanyl)-5-[2-(triisopropylsilyl)ethynyl]pyrido[2,3-d]pyrimidin-7-yl]urea

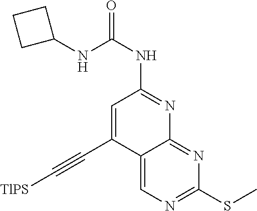

General Procedure 7 was applied to 2-(methylsulfanyl)-5-[2-(triisopropylsilyl)ethynyl]pyrido[2,3-d]pyrimidin-7-amine (600 mg, 1.61 mmol), cyclobutylamine (229 mg, 3.22 mmol), triethylamine (244 mg, 2.41 mmol) and CDI (392 mg, 2.41 mmol) in dichloromethane. The residue was purified by flash column chromatography eluting with ethyl acetate (6%) in 40-60 petroleum ether to afford as a light yellow solid (450 mg, 59%).

(ES, m/z): [M+H]⁺=467.2

### 3-cyclobutyl-1-{2-methanesulfonyl-5-[2-(triisopropylsilyl)ethynyl]pyrido[2,3-d]pyrimidin-7-yl}urea

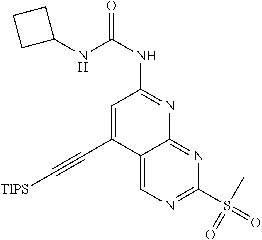

General Procedure 2 was applied to 3-cyclobutyl-1-[2-(methylsulfanyl)-5-[2-(triisopropylsilyl)ethynyl]pyrido[2,3-d]pyrimidin-7-yl]urea (450 mg, 0.958 mmol) and m-CPBA (364 mg, 2.11 mmol) in dichloromethane (5 mL) to yield the title compound as a brown solid (470 mg, 98%).

(ES, m/z): [M+H]⁺=502.2

### 3-cyclobutyl-1-(2-{[4-(4-methylpiperazin-1-yl)phenyl]amino}-5-[2-(triisopropylsilyl)ethynyl]pyrido[2,3-d]pyrimidin-7-yl)urea

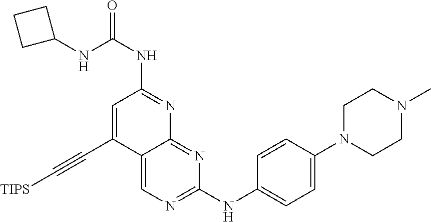

General Procedure 3 was applied to 3-cyclobutyl-1-{2-methanesulfonyl-5-[2-(triisopropylsilyl)ethynyl]pyrido[2,3-d]pyrimidin-7-yl}urea (400 mg, 0.797 mmol) and 4-(4-methylpiperazin-1-yl)aniline (229 mg, 1.19 mmol) and trifluoroacetic acid (182 mg, 1.59 mmol) in 2-butanol (1 mL). The resulting mixture was stirred for 2 h at 100° C. The residue was purified by reverse phase flash column chromatography eluting with acetonitrile (10-50%) in water to yield the title compound as an orange red solid (180 mg, 37%).

(ES, m/z): [M+H]⁺=613.4

### 3-cyclobutyl-1-(5-ethynyl-2-{[4-(4-methylpiperazin-1-yl)phenyl]amino}pyrido[2,3-d]pyrimidin-7-yl)urea

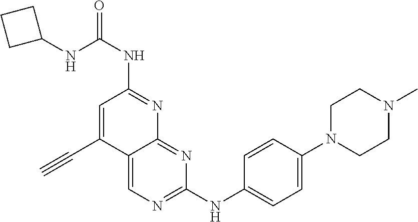 [SMILES: C#Cc1cc(NC(=O)NC2CCC2)nc2nc(Nc3ccc(N4CCN(C)CC4)cc3)ncc12]

General Procedure 1 was applied to 3-cyclobutyl-1-(2-{[4-(4-methylpiperazin-1-yl)phenyl]amino}-5-[2-(triisopropylsilyl)ethynyl]pyrido[2,3-d]pyrimidin-7-yl)urea (180 mg, 0.294 mmol) and potassium fluoride (171 mg, 2.94 mmol) in THF (2 mL), DMF (2 mL) and water (180 uL). The residue was purified by reverse phase flash column chromatography eluting with acetonitrile (10-50%) in water to yield the title compound as an orange solid (28.0 mg, 21%)

(ES, m/z): [M+H]⁺=457.2

¹H NMR (400 MHz, DMSO-d₆) δ 9.99 (d, J=23.4 Hz, 2H), 9.07 (s, 1H), 8.14 (s, 1H), 7.81 (s, 2H), 7.32 (s, 1H), 6.92 (d, J=9.0 Hz, 2H), 5.05 (s, 1H), 4.22 (q, J=7.8 Hz, 1H), 3.13 (s, 4H), 2.33 (d, J=20.5 Hz, 5H), 2.01-1.87 (m, 2H), 1.72 (dt, J=18.4, 8.7 Hz, 2H).

### Example 42

### 2-methanesulfonyl-5-[2-(triisoprooylsilyl)ethynyl]pyrido[2,3-d]pyrimidin-7-amine

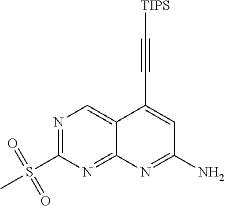

General Procedure 2 was applied to 2-(methylsulfanyl)-5-[2-(triisopropylsilyl)ethynyl]pyrido[2,3-d]pyrimidin-7-amine (1.80 g, 4.83 mmol) and m-CPBA (1.25 g, 7.25 mmol) in dichloromethane (20 mL) to yield the title compound as a yellow solid (1.50 g, 77%).

(ES, m/z): [M+H]⁺=405.6

### N²-[4-(4-methylDiperazin-1-yl)phenyl]-5-[2-(triisoproDvisilyl)ethynyl]pyrido[2,3-d]pyrimidine-2,7-diamine

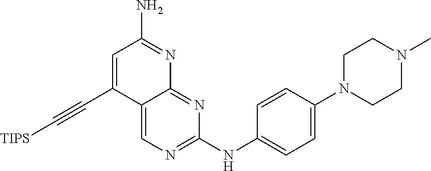

General Procedure 3 was applied to 2-methanesulfonyl-5-[2-(triisopropylsilyl)ethynyl]pyrido[2,3-d]pyrimidin-7-amine (600 mg, 1.48 mmol), 4-(4-methylpiperazin-1-yl)aniline (425 mg, 2.22 mmol) and trifluoroacetic acid (0.220 mL, 2.96 mmol) in 2-butanol (8 mL). The reaction mixture was stirred for 2 h at 100° C. The residue was purified by reverse phase flash column chromatography eluting with acetonitrile (10-100%) in water (0.1% formic acid) to yield the title compound as a red oil (389 mg, 51%).

(ES, m/z): [M+H]⁺=516.7

### 1-(2-{[4-(4-methylpiperazin-1-yl)phenyl]amino}-5-[2-(triisoprooylsilyl)ethynyl]pyrido[2,3-d]pyrimidin-7-yl)-3-(oxolan-3-yl)urea

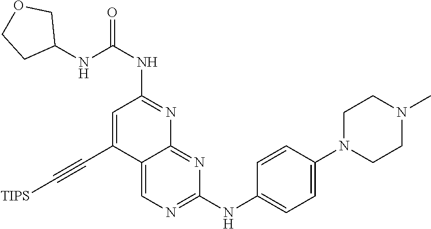

General Procedure 7 was applied to N²-[4-(4-methylpiperazin-1-yl)phenyl]-5-[2-(triisopropylsilyl)ethynyl]pyrido[2,3-d]pyrimidine-2,7-diamine (420 mg, 0.814 mmol), CDI (2.30 mL, 1.63 mmol), potassium carbonate (169 mg, 1.22 mmol) and oxolan-3-amine (142 mg, 1.63 mmol) in dichloromethane (5 mL) to yield the title compound as a yellow solid (187 mg, 36%).

(ES, m/z): [M+H]⁺=629.9

### 1-(5-ethynyl-2-{[4-(4-methylpiperazin-1-yl)phenyl]amino}pyrido[2,3-d]pyrimidin-7-yl)-3-(oxolan-3-yl)urea

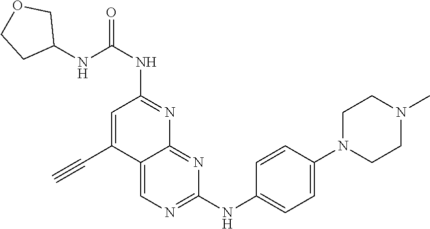 [SMILES: C#Cc1cc(NC(=O)NC2CCOC2)nc2nc(Nc3ccc(N4CCN(C)CC4)cc3)ncc12]

General Procedure 1 was applied to 1-(2-{[4-(4-methylpiperazin-1-yl)phenyl]amino}-5-[2-(triisopropylsilyl)ethynyl]pyrido[2,3-d]pyrimidin-7-yl)-3-(oxolan-3-yl)urea (153 mg, 0.243 mmol) and potassium fluoride (141 mg, 2.43 mmol) in THF (3 mL), water (1 mL) and DMF (3 mL). The reaction mixture was stirred for 2 h at 35° C. The residue was purified by reverse phase flash column chromatography eluting with acetonitrile (10-50%) in water (0.1% formic acid) to yield the title compound as a yellow solid (43.0 mg, 37%).

(ES, m/z): [M+H]⁺=473.5

¹H NMR (300 MHz, DMSO-d₆) δ 10.05 (d, J=32.3 Hz, 2H), 9.56 (s, 1H), 9.07 (s, 1H), 7.84 (s, 2H), 7.21 (s, 1H), 6.93 (d, J=8.8 Hz, 2H), 5.06 (s, 1H), 4.37 (d, J=9.6 Hz, 1H), 4.01-3.77 (m, 3H), 3.63 (dd, J=9.0, 2.7 Hz, 1H), 3.16 (s, 4H), 2.73 (s, 4H), 2.42 (s, 3H), 2.24 (dt, J=12.7, 7.7 Hz, 1H), 1.92-1.78 (m, 1H).

### Example 43

### 1-(2-{[4-(4-methylpiperazin-1-yl)phenyl]amino}-5-[2-(triisoprooylsilyl)ethynyl]pyrido[2,3-d]pyrimidin-7-yl)-3-(oxetan-3-yl)urea

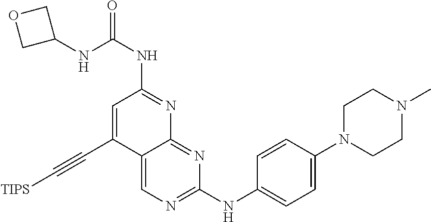

General Procedure 7 was applied to N²-[4-(4-methylpiperazin-1-yl)phenyl]-5-[2-(triisopropylsilyl) ethynyl]pyrido[2,3-d]pyrimidine-2,7-diamine (200 mg, 0.388 mmol), CDI (94.3 mg, 0.582 mmol), triethylamine (58.8 mg, 0.582 mmol) and oxetan-3-amine (56.7 mg, 0.776 mmol) in dichloromethane to yield the title compound as a red solid (120 mg, 50%).

### 1-(5-ethynyl-2-{[4-(4-methylpiperazin-1-yl)phenyl]amino}pyrido[2,3-d]pyrimidin-7-yl)-3-(oxetan-3-ylI)urea

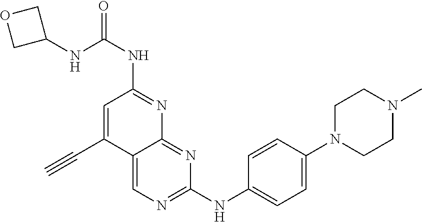 [SMILES: C#Cc1cc(NC(=O)NC2COC2)nc2nc(Nc3ccc(N4CCN(C)CC4)cc3)ncc12]

General Procedure 1 was applied to 1-(2-{[4-(4-methylpiperazin-1-yl)phenyl]amino}-5-[2-(triisopropylsilyl)ethynyl]pyrido[2,3-d]pyrimidin-7-yl)-3-(oxetan-3-yl)urea (120 mg, 0.195 mmol) and potassium fluoride (113 mg, 1.95 mmol) in THF (2 mL), DMF (2 mL) and water (0.5 mL). The resulting solution was stirred for 2 h at 70° C. The reaction mixture was purified by reverse phase flash column chromatography eluting with acetonitrile (20-100%) in water (0.1% trifluoroacetic acid). The desired fractions were collected and concentrated under reduced pressure to remove most of the acetonitrile then lyophilizied to yield the title compound as an orange solid (43.6 mg, 48.72%).

(ES, m/z): [M+H]⁺=459

¹H-NMR: (300 MHz, DMSO-d₆): δ 10.23 (s, 1H), 9.98 (s, 1H), 9.45 (s, 1H), 9.09 (s, 1H), 7.79 (s, 2H), 7.33 (s, 1H), 6.92 (d, J=9.1 Hz, 2H), 5.06 (s, 1H), 4.85 (d, J=3.0 Hz, 3H), 4.51 (s, 2H), 3.09 (t, J=5.0 Hz, 4H), 2.46 (t, J=5.0 Hz, 4H), 2.23 (s, 3H).

### Example 44

### 3-cyclopropyl-1-[2-(methylsulfanyl)-5-[2-(triisoproovisilyl)ethynyl]pyrido[2,3-d]pyrimidin-7-yl]urea

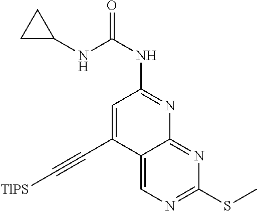

General Procedure 7 was applied to 2-(methylsulfanyl)-5-[2-(triisopropylsilyl)ethynyl]pyrido[2,3-d]pyrimidin-7-amine (700 mg, 7.88 mmol), triethylamine (390 uL, 2.82 mmol), CDI (609 mg, 3.76 mmol) and 1-cyclopentylmethanamine (215 mg, 3.76 mmol) in dichloromethane (7 mL). The residue was purified by flash column chromatography eluting with ethyl acetate (20%) in 40-60 petroleum ether to afford the title compound as a white solid (700 mg, 82%).

(ES, m/z): [M+H]⁺=456

### 3-cyclopropyl-1-{2-methanesulfonyl-5-[2-(triisopropylsilyl)ethynyl]pyrido[2,3-d]pyrimidin-7-yl}urea

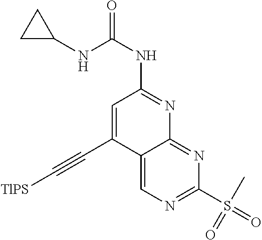

General Procedure 2 was applied to 3-cyclopropyl-1-[2-(methylsulfanyl)-5-[2-(triisopropylsilyl)ethynyl]pyrido[2,3-d]pyrimidin-7-yl]urea (400 mg, 0.878 mmol) and m-CPBA (333 mg, 1.93 mmol) in dichloromethane (4 mL) to yield the title compound as a brown solid (664 mg, 155%).

(ES, m/z): [M+H]⁺=488

### 3-cyclopropyl-1-(2-{[4-(4-methylpiperazin-1-yl)phenyl]amino}-5-[2-(triisopropylsilyl)ethynyl]pyrido[2,3-d]pyrimidin-7-yl)urea

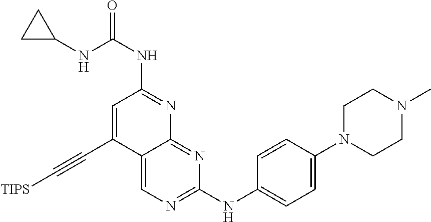

General Procedure 3 was applied to 3-cyclopropyl-1-{2-methanesulfonyl-5-[2-(triisopropylsilyl) ethynyl]pyrido[2,3-d]pyrimidin-7-yl}urea (466 mg, 0.957 mmol), 4-(4-methylpiperazin-1-yl) aniline (274 mg, 1.44 mmol) and trifluoroacetic acid (142 uL, 1.91 mmol) in 2-butanol (4.66 mL). The resulting mixture was stirred for 1 h at 100° C. The residue was purified by reverse phase flash column chromatography eluting with acetonitrile (10-50%) in water (0.1% trifluoroacetic acid) to yield the title compound as a brown solid (359 mg, 63%).

(ES, m/z): [M+H]⁺=599

### 3-cyclopropyl-1-(5-ethynyl-2-{[4-(4-methylpiperazin-1-yl)phenyl]amino}pyrido[2,3-d]pyrimidin-7-yl)urea

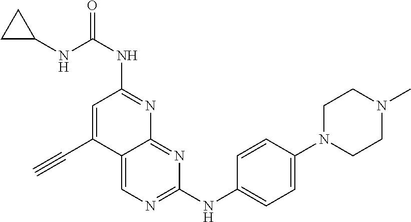 [SMILES: C#Cc1cc(NC(=O)NC2CC2)nc2nc(Nc3ccc(N4CCN(C)CC4)cc3)ncc12]

General Procedure 1 was applied to 3-cyclopropyl-1-(2-{[4-(4-methylpiperazin-1-yl)phenyl]amino}-5-[2-(triisopropylsilyl)ethynyl]pyrido[2,3-d]pyrimidin-7-yl)urea (315 mg, 0.526 mmol) and potassium fluoride (305 mg, 5.26 mmol) in THF (1.6 mL), DMF (1.5 mL) and water (126 uL). The resulting mixture was stirred for 1 h at 60° C. The residue was purified by reverse phase flash column chromatography eluting with acetonitrile (10-50%) in water (0.1% formic acid) to afford the title compound as a yellow solid (130 mg, 56%)

(ES, m/z): [M+H]⁺=443

¹H NMR (400 MHz, DMSO-d₆) δ 10.05 (s, 1H), 9.95 (s, 1H), 9.06 (s, 1H), 7.77 (s, 2H), 7.32 (s, 1H), 6.98-6.82 (m, 2H), 3.18-3.02 (m, 4H), 2.76-2.62 (m, 1H), 2.47 (t, J=5.0 Hz, 4H), 2.23 (s, 3H), 0.76 (td, J=7.0, 4.9 Hz, 2H), 0.57-0.40 (m, 2H).

### Example 45

### 1-(2-{[4-(4-methylpiperazin-1-yl)phenyl]amino}-5-[2-(triisopropylsilyl)ethynyl]pyrido[2,3-d]pyrimidin-7-yl)-3-(1,3-oxazol-5-ylmethyl)urea

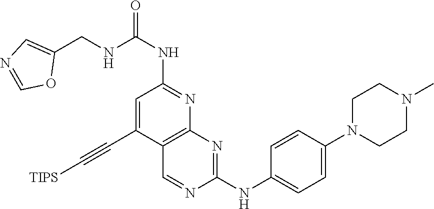

General Procedure 7 was applied to N²-[4-(4-methylpiperazin-1-yl)phenyl]-5-[2-(triisopropylsilyl)ethynyl]pyrido[2,3-d]pyrimidine-2,7-diamine (300 mg, 0.582 mmol), triethylamine (118 mg, 1.16 mmol), CDI (189 mg, 1.16 mmol) and 1-(1,3-oxazol-5-yl)methanamine (114 mg, 1.16 mmol) in dichloromethane (21 mL). The residue was purified by flash column chromatography eluting with ethyl acetate (5%) in 40-60 petroleum ether to afford the title compound as an orange solid (230 mg, 62%).

(ES, m/z): [M+H]⁺=640.3

### 1-(5-ethynyl-2-{[4-(4-methylpiperazin-1-yl)phenyl]amino}pyrido[2,3-d]pyrimidin-7-yl)-3-(1,3-oxazol-5-ylmethyl)urea

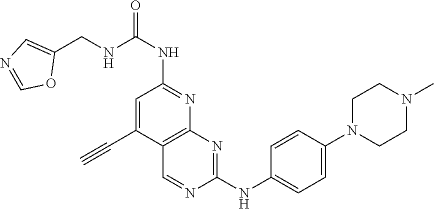 [SMILES: C#Cc1cc(NC(=O)NCc2cnco2)nc2nc(Nc3ccc(N4CCN(C)CC4)cc3)ncc12]

General Procedure 1 was applied to 1-(2-{[4-(4-methylpiperazin-1-yl)phenyl]amino}-5-[2-(triisopropylsilyl)ethynyl]pyrido[2,3-d]pyrimidin-7-yl)-3-(1,3-oxazol-5-ylmethyl)urea (200 mg, 0.313 mmol) and potassium fluoride (182 mg, 3.13 mmol) in THE (4 mL), DMF (4 mL) and water (200 uL). The reaction mixture was stirred for 2 h at 60° C. The residue was purified by reverse phase flash column chromatography eluting with acetonitrile (10-30%) in water (0.1% trifluoroacetic acid) to yield the title compound as an orange solid (58.4 mg, 38.64%).

(ES, m/z): [M+H]⁺=484.2

¹H NMR (400 MHz, DMSO-d₆) δ 10.26 (s, 1H), 9.96 (s, 1H), 9.25 (s, 1H), 9.07 (s, 1H), 8.35 (s, 1H), 7.74 (s, 2H), 7.28 (s, 1H), 7.17 (s, 1H), 6.98-6.88 (m, 2H), 5.05 (s, 1H), 4.57 (d, J=5.4 Hz, 2H), 3.11 (t, J=5.0 Hz, 4H), 2.54 (s, 2H), 2.25 (s, 3H).

### Example 46

### 3-[(3-methylmidazol-4-yl)methyl]-1-(2-{[4-(4-methylpiperazin-1-yl)phenyl]amino}-5-[2-(triisopropylsilyl)ethynyl]pyrido[2,3-d]pyrimidin-7-yl)urea

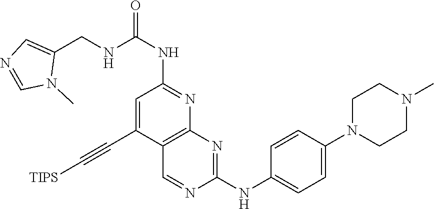

General Procedure 7 was applied to N²-[4-(4-methylpiperazin-1-yl)phenyl]-5-[2-(triisopropylsilyl)ethynyl]pyrido[2,3-d]pyrimidine-2,7-diamine (20.0 mg, 39.0 μmol), triethylamine (7.85 mg, 78.0 μmol) and 1-(3-methylmidazol-4-yl)methanamine (8.62 mg, 78.0 μmol) in DMF (1 mL). The residue was purified by reverse phase flash column chromatography eluting with acetonitrile (10-50%) in water (0.1% trifluoroacetic acid) to yield the title compound as a yellow solid (160 mg, 25%).

### 1-(5-ethynyl-2-{[4-(4-methylpiperazin-1-yl)phenyl]amino}pyrido[2,3-d]pyrimidin-7-yl)-3-[(3-methylmidazol-4-yl)methyl]urea

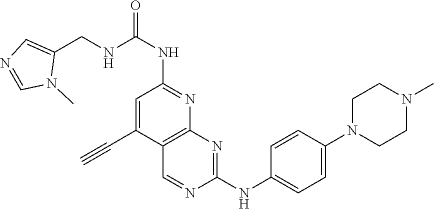 [SMILES: C#Cc1cc(NC(=O)NCc2cncn2C)nc2nc(Nc3ccc(N4CCN(C)CC4)cc3)ncc12]

General Procedure 1 was applied to 3-[(3-methylmidazol-4-yl)methyl]-1-(2-{[4-(4-methylpiperazin-1-yl)phenyl]amino}-5-[2-(triisopropylsilyl)ethynyl]pyrido[2,3-d]pyrimidin-7-yl)urea (160 mg, 0.245 mmol) and potassium fluoride (142 mg, 2.45 mmol) in THF (2 mL), DMF (2 mL) and water (160 uL). The reaction imxture was stirred for 2 h at 60° C. The residue was purified by reverse phase flash column chromatography eluting with acetonitrile (10-50%) in water (0.1% trifluoroacetic acid) to yield the title compound as an orange solid (55.4 mg, 45%).

(ES, m/z): [M+H]⁺=497.2

¹H-NMR (400 MHz, DMSO-d₆) δ 10.20 (s, 1H), 9.94 (s, 1H), 9.50 (s, 1H), 9.05 (s, 1H), 7.74 (d, J=8.2 Hz, 2H), 7.59 (s, 1H), 7.17 (s, 1H), 7.01-6.92 (m, 3H), 5.05 (s, 1H), 4.50 (d, J=5.3 Hz, 2H), 3.69 (s, 4H), 3.13 (t, J=4.9 Hz, 4H), 2.48 (d, J=4.8 Hz, 3H), 2.24 (s, 3H).

### Example 47

### 1-(2-{[4-(4-methylpiperazin-1-yl)phenyl]amino}-5-[2-(triisopropylsilyl)ethynyl]pyrido[2,3-d]pyrimidin-7-yl)-3-(1,2-oxazol-5-ylmethyl)urea

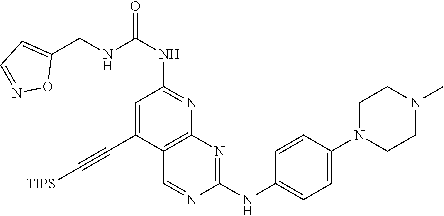

General Procedure 7 was applied to N²-[4-(4-methylpiperazin-1-yl)phenyl]-5-[2-(triisopropylsilyl)ethynyl]pyrido[2,3-d]pyrimidine-2,7-diamine (300 mg, 0.582 mmol), triethylamine (118 mg, 1.16 mmol), CDI (189 mg, 1.16 mmol) and 1-(1,2-oxazol-5-yl)methanamine (114 mg, 1.16 mmol) in dichloromethane (21 mL). The residue was purified by reverse phase flash column chromatography eluting with acetonitrile (10-50%) in water (0.1% trifluoroacetic acid) to yield the title compound as an orange solid (170 mg, 46%).

(ES, m/z): [M+H]⁺=640.3

### 1-(5-ethynyl-2-{[4-(4-methylpiperazin-1-yl)phenyl]amino}pyrido[2,3-d]pyrimidin-7-yl)-3-(1,2-oxazol-5-ylmethyl)urea

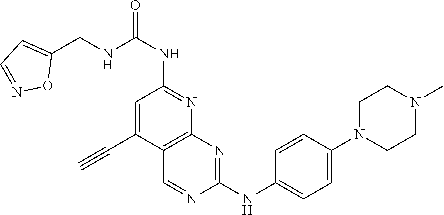 [SMILES: C#Cc1cc(NC(=O)NCc2ccno2)nc2nc(Nc3ccc(N4CCN(C)CC4)cc3)ncc12]

General Procedure 1 was applied to 1-(2-{[4-(4-methylpiperazin-1-yl)phenyl]amino}-5-[2-(triisopropylsilyl)ethynyl]pyrido[2,3-d]pyrimidin-7-yl)-3-(1,2-oxazol-5-ylmethyl)urea (160 mg, 0.250 mmol) and potassium fluoride (145 mg, 2.50 mmol) in THE (4 mL), DMF (4 mL) and water (160 uL). The reaction mixture was stirred for 2 h at 60° C. The residue was purified by reverse phase flash column chromatography eluting with acetonitrile (10-30%) in water (0.1% trifluoroacetic acid) to yield the title compound as an orange solid (43.9 mg, 36%).

(ES, m/z): [M+H]⁺=484.2

¹H NMR (400 MHz, DMSO-d₆) δ 10.32 (s, 1H), 9.97 (s, 1H), 9.08 (s, 1H), 8.52 (d, J=1.8 Hz, 1H), 7.76 (s, 2H), 7.33 (s, 1H), 6.97-6.86 (m, 2H), 6.46 (d, J=1.8 Hz, 1H), 5.06 (s, 1H), 4.67 (d, J=5.6 Hz, 2H), 3.10 (t, J=5.0 Hz, 4H), 2.26 (s, 3H).

### Example 48

### 1-(2-{[4-(4-methylpiperazin-1-yl)phenyl]amino}-5-[2-(triisopropylsilyl)ethynyl]pyrido[2,3-d]pyrimidin-7-yl)-3-[(2-methylpyrazol-3-yl)methyl]urea

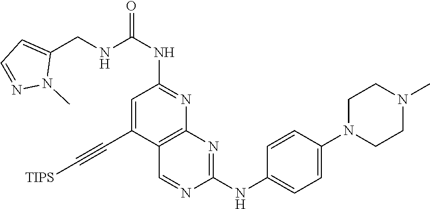

General Procedure 7 was applied to N²-[4-(4-methylpiperazin-1-yl)phenyl]-5-[2-(triisopropylsilyl)ethynyl]pyrido[2,3-d]pyrimidine-2,7-diamine (300 mg, 0.582 mmol), triethylamine (118 mg, 1.16 mmol), CDI (189 mg, 1.16 mmol) and 1-(2-methylpyrazol-3-yl)methanamine (129 mg, 1.16 mmol) in dichloromethane (21 mL). The residue was purified by reverse phase flash column chromatography eluting with acetonitrile (10-50%) in water (0.1% formic acid) to yield the title compound as an orange solid (300 mg, 79%).

(ES, m/z): [M+H]⁺=653.4

### 1-(5-ethynyl-2-{[4-(4-methylpiperazin-1-yl)phenyl]amino}pyrido[2,3-d]pyrimidin-7-yl)-3-[(2-methylpyrazol-3-yl)methyl]urea

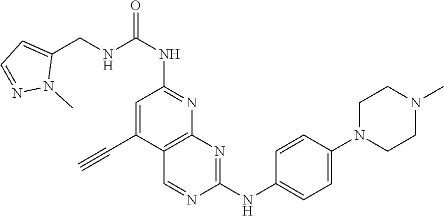 [SMILES: C#Cc1cc(NC(=O)NCc2ccnn2C)nc2nc(Nc3ccc(N4CCN(C)CC4)cc3)ncc12]

General Procedure 1 was applied to 1-(2-{[4-(4-methylpiperazin-1-yl)phenyl]amino}-5-[2-(triisopropylsilyl)ethynyl]pyrido[2,3-d]pyrimidin-7-yl)-3-[(2-methylpyrazol-3-yl)methyl]urea (280 mg, 0.429 mmol) and potassium fluoride (249 mg, 4.29 mmol) in THF (5 mL), DMF (5 mL) and water (280 uL). The reaction mixture was stirred for 2 h at 60° C. The residue was purified by reverse phase flash column chromatography eluting with acetonitrile (10-30%) in water (0.1% trifluoroacetic acid) to yield the title compound as a yellow solid (49.7 mg, 23%).

(ES, m/z): [M+H]⁺=497

¹H NMR (400 MHz, DMSO-d₆) δ 10.26 (s, 1H), 9.98 (s, 1H), 9.52 (s, 1H), 9.07 (s, 1H), 7.77 (s, 2H), 7.35 (d, J=1.9 Hz, 1H), 7.21 (s, 1H), 6.97 (d, J=8.5 Hz, 2H), 6.31 (s, 1H), 5.06 (s, 1H), 4.57 (d, J=5.4 Hz, 2H), 3.88 (s, 3H), 2.89 (s, 4H).

### Example 49

### 3-(2-{[4-(4-methylpiperazin-1-yl)phenyl]amino}-5-[2-(triisopropylsilyl)ethynyl]pyrido[2,3-d]pyrimidin-7-yl)-1-(pyridin-2-ylmethyl)urea

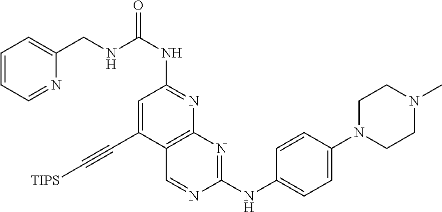

General Procedure 7 was applied to N²-[4-(4-methylpiperazin-1-yl)phenyl]-5-[2-(triisopropylsilyl)ethynyl]pyrido[2,3-d]pyrimidine-2,7-diamine (30.0 mg, 58.0 μmol), triethylamine (8.83 mg, 87.0 μmol) and CDI (14.2 mg, 87.0 μmol) and 2-pyridinemethaneamine (12.6 mg, 0.116 mmol) in dichloromethane (1 mL). The residue was purified by reverse phase flash column chromatography eluting with acetonitrile (10-50%) in water (0.1% trifluoroacetic acid) to yield the title compound as a yellow solid (300 mg, 48%).

(ES, m/z): [M+H]⁺=650.4

### 3-(5-ethynyl-2-{[4-(4-methylpiperazin-1-yl)phenyl]amino}pyrido[2,3-d]pyrimidin-7-yl)-1-(pyridin-2-ylmethyl)urea

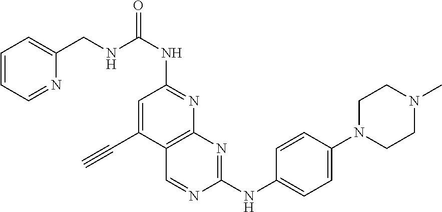 [SMILES: C#Cc1cc(NC(=O)NCc2ccccn2)nc2nc(Nc3ccc(N4CCN(C)CC4)cc3)ncc12]

General Procedure 1 was applied to 3-(2-{[4-(4-methylpiperazin-1-yl)phenyl]amino}-5-[2-(triisopropylsilyl)ethynyl]pyrido[2,3-d]pyrimidin-7-yl)-1-(pyridin-2-ylmethyl)urea (230 mg, 0.354 mmol) and potassium fluoride (206 mg, 3.54 mmol) in THF (3 mL), DMF (3 mL) and water (230 uL). The reaction mixture was stirred for 2 h at 60° C. The residue was purified by reverse phase flash column chromatography eluting with acetonitrile (10-50%) in water (0.1% trifluoroacetic acid) to yield the title compound as an orange solid (36.4 mg, 21%).

(ES, m/z): [M+H]⁺=494.2

¹H NMR (400 MHz, DMSO-d₆) δ 10.26 (s, 1H), 9.94 (s, 1H), 9.30 (s, 1H), 9.08 (s, 1H), 8.55 (d, J=5.4 Hz, 1H), 7.79 (td, J=7.7, 1.8 Hz, 3H), 7.49-7.23 (m, 3H), 6.87 (d, J=8.6 Hz, 2H), 5.04 (s, 1H), 4.59 (d, J=5.6 Hz, 2H), 3.10 (t, J=5.0 Hz, 4H), 2.54 (d, J=5.4 Hz, 4H), 2.28 (s, 3H).

### Example 50

### 1-(2-{[4-(4-methylpiperazin-1-yl)phenyl]amino}-5-[2-(triisopropylsilyl)ethynyl]pyrido[2,3-d]pyrimidin-7-yl)-3-(oxolan-2-ylmethyl)urea

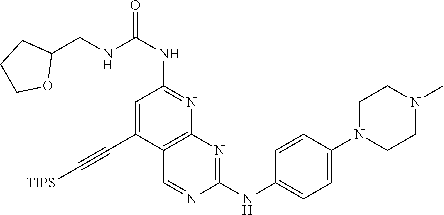

General Procedure 7 was applied to N²-[4-(4-methylpiperazin-1-yl)phenyl]-5-[2-(triisopropylsilyl)ethynyl]pyrido[2,3-d]pyrimidine-2,7-diamine (300 mg, 580 μmol), triethylamine (118 mg, 1.16 mmol), CDI (189 mg, 1.16 mmol) and 1-(oxolan-2-yl)methanamine (118 mg, 1.16 mmol) in dichloromethane (10 mL). The residue was purified by flash column chromatography eluting with ethyl acetate (3%) in 40-60 petroleum ether to afford the title compound as an orange solid (230 mg, 62%).

### 1-(5-ethynyl-2-{[4-(4-methylpiperazin-1-yl)phenyl]amino}pyrido[2,3-d]pyrimidin-7-yl)-3-(oxolan-2-ylmethyl)urea

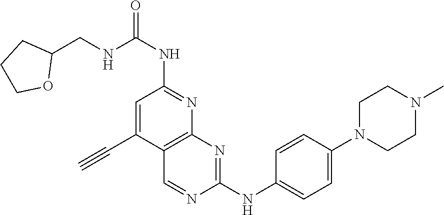 [SMILES: C#Cc1cc(NC(=O)NCC2CCCO2)nc2nc(Nc3ccc(N4CCN(C)CC4)cc3)ncc12]

General Procedure 1 was applied to 1-(2-{[4-(4-methylpiperazin-1-yl)phenyl]amino}-5-[2-(triisopropylsilyl)ethynyl]pyrido[2,3-d]pyrimidin-7-yl)-3-(oxolan-2-ylmethyl)urea (200 mg, 0.311 mmol) and potassium fluoride (181 mg, 3.11 mmol) in THF (4 mL), DMF (4 mL) and water (200 uL). The reaction mixture was stirred for 2 h at 60° C. The residue was purified by reverse phase flash column chromatography eluting with acetonitrile (10-30%) in water (0.1% trifluoroacetic acid) to yield the title compound as a yellow solid (70.9 mg, 47%).

(ES, m/z): [M+H]⁺=487.25

¹H-NMR (400 MHz, DMSO-d₆) δ 10.10 (s, 1H), 9.94 (s, 1H), 9.06 (s, 2H), 7.80 (s, 2H), 7.26 (s, 1H), 6.90 (d, J=8.7 Hz, 2H), 5.04 (s, 1H), 3.97 (p, J=6.1 Hz, 1H), 3.85 (t, J=7.0 Hz, 1H), 3.66 (q, J=7.3 Hz, 2H), 3.08 (t, J=4.9 Hz, 4H), 2.49-2.40 (m, 4H), 2.23 (s, 3H), 1.99 (dq, J=12.5, 6.7, 6.2 Hz, 1H), 1.84 (tq, J=12.3, 6.0, 5.3 Hz, 2H), 1.62 (dq, J=14.9, 7.3 Hz, 1H).

### Example 51

### 1-(2-{[4-(4-methylpiperazin-1-yl)phenyl]amino}-5-[2-(triisopropylsilyl)ethynyl]pyrido[2,3-d]pyrimidin-7-yl)-3-(oxolan-3-ylmethyl)urea

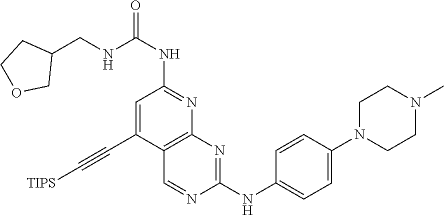

General Procedure 7 was applied to N²-[4-(4-methylpiperazin-1-yl)phenyl]-5-[2-(triisopropylsilyl)ethynyl]pyrido[2,3-d]pyrimidine-2,7-diamine (300 mg, 0.582 mmol), triethylamine (118 mg, 1.16 mmol), CDI (189 mg, 1.16 mmol) and 1-(oxolan-3-yl)methanamine (118 mg, 1.16 mmol) in dichloromethane (21 mL). The residue was purified by reverse phase flash column chromatography eluting with acetonitrile (10-50%) in water (0.1% trifluoroacetic acid) to yield the title compound as an orange solid (180 mg, 48%).

(ES, m/z): [M+H]⁺=643.4

### 1-(5-ethynyl-2-{[4-(4-methylpiperazin-1-yl)phenyl]amino}pyrido[2,3-d]pyrimidin-7-yl)-3-(oxolan-3-ylmethyl)urea

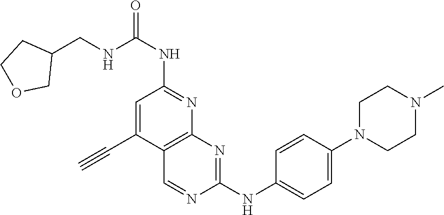 [SMILES: C#Cc1cc(NC(=O)NCC2CCOC2)nc2nc(Nc3ccc(N4CCN(C)CC4)cc3)ncc12]

General Procedure 1 was applied to 1-(2-{[4-(4-methylpiperazin-1-yl)phenyl]amino}-5-[2-(triisopropylsilyl)ethynyl]pyrido[2,3-d]pyrimidin-7-yl)-3-(oxolan-3-ylmethyl)urea (170 mg, 0.264 mmol) and potassium fluoride (154 mg, 2.64 mmol) in THF (4 mL), DMF (4 mL) and water (170 uL). The reaction mixture was stirred for 2 h at 60° C. The residue was purified by reverse phase flash column chromatography eluting with acetonitrile (10-30%) in water (0.1% trifluoroacetic acid) to yield the title compound as a yellow solid (72.1 mg, 56%).

(ES, m/z): [M+H]⁺=487.2

¹H NMR (400 MHz, DMSO-d₆) δ 10.12 (s, 1H), 9.96 (s, 1H), 9.36 (s, 1H), 9.06 (s, 1H), 7.81 (s, 2H), 7.18 (s, 1H), 6.92 (d, J=8.9 Hz, 2H), 5.04 (s, 1H), 3.91-3.76 (m, 2H), 3.69 (t, J=7.6 Hz, 1H), 3.50 (dd, J=8.6, 6.0 Hz, 1H), 3.30 (h, J=7.0 Hz, 3H), 3.10 (t, J=5.0 Hz, 4H), 2.53 (s, 3H), 2.45 (p, J=6.8 Hz, 1H), 2.27 (d, J=4.5 Hz, 3H), 2.07 (dt, J=16.0, 6.6 Hz, 1H), 1.67 (dq, J=14.0, 7.1 Hz, 1H).

### Example 52

### 3-[(3R)-1-acetylpyrrolidin-3-yl]-1-(2-{[4-(4-methylpiperazin-1-yl)phenyl]amino}-5-[2-(triisopropylsilyl)ethynyl]pyrido[2,3-d]pyrimidin-7-yl) urea

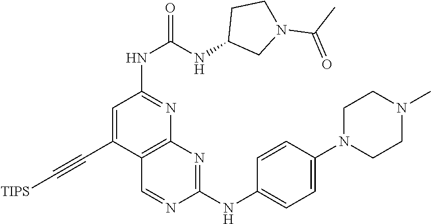

General Procedure 7 was applied to N²-[4-(4-methylpiperazin-1-yl)phenyl]-5-[2-(triisopropylsilyl)ethynyl]pyrido[2,3-d]pyrimidine-2,7-diamine (300 mg, 0.582 mmol), triethylamine (118 mg, 1.16 mmol), CDI (189 mg, 1.16 mmol) and 1-[(3R)-3-aminopyrrolidin-1-yl]ethanone (149 mg, 1.16 mmol) in dichloromethane (20 mL). The residue was purified by reverse phase flash column chromatography eluting with acetonitrile (10-50%) in water (0.1% trifluoroacetic acid) to yield the title compound as a yellow solid (300 mg, 77%)

(ES, m/z): [M+H]⁺=670.4

### 3-[(3R)-1-acetylpyrrolidin-3-yl]-1-(5-ethynyl-2-{[4-(4-methylpiperazin-1-yl)phenyl]amino}pyrido[2,3-d]pyrimidin-7-ylurea

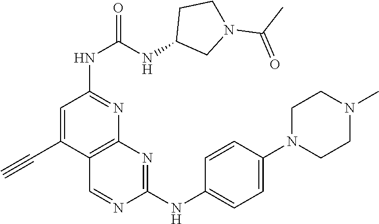 [SMILES: C#Cc1cc(NC(=O)N[C@@H]2CCN(C(C)=O)C2)nc2nc(Nc3ccc(N4CCN(C)CC4)cc3)ncc12]

General Procedure 1 was applied to 3-[(3R)-1-acetylpyrrolidin-3-yl]-1-(2-{[4-(4-methylpiperazin-1-yl)phenyl]amino}-5-[2-(triisopropylsilyl)ethynyl]pyrido[2,3-d]pyrimidin-7-yl)urea (300 mg, 0.448 mmol) and potassium fluoride (260 mg, 4.48 mmol) in THF (3 mL), DMF (3 mL) and water (300 uL). The reaction mixture was stirred for 2 h at 60° C. The residue was purified by reverse phase flash column chromatography eluting with acetonitrile (10-50%) in water (0.1% trifluoroacetic acid) to yield the title compound as an orange solid (50.0 mg, 22%).

(ES, m/z): [M+H]⁺=514.10

¹H NMR (400 MHz, DMSO-d₆) δ 10.12 (d, J=10.1 Hz, 1H), 9.96 (s, 1H), 9.56 (s, 1H), 9.06 (s, 1H), 7.79 (s, 2H), 7.19 (s, 1H), 6.97 (dd, J=13.7, 8.9 Hz, 2H), 5.05 (s, 1H), 4.48-4.22 (m, 1H), 3.81-3.48 (m, 4H), 3.11 (t, J=5.0 Hz, 4H), 2.48 (s, 3H), 2.35-2.26 (m, 1H), 2.24 (s, 3H), 2.16 (dt, J=14.2, 7.0 Hz, 1H), 1.99 (d, J=5.9 Hz, 1H), 1.93 (d, J=10.2 Hz, 3H), 1.87 (dd, J=12.4, 6.1 Hz, 1H).

### Example 53

### 3-[(3S)-1-acetylpyrrolidin-3-yl]-1-(2-{[4-(4-methylpiperazin-1-yl)phenyl]amino}-5-[2-(triisopropylsilyl)ethynyl]pyrido[2,3-d]pyrimidin-7-yl)urea

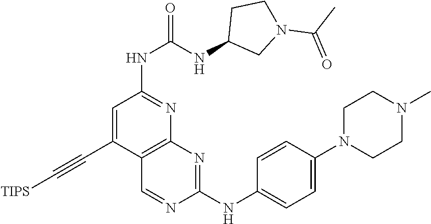

General Procedure 7 was applied to N²-[4-(4-methylpiperazin-1-yl)phenyl]-5-[2-(triisopropylsilyl)ethynyl]pyrido[2,3-d]pyrimidine-2,7-diamine (300 mg, 0.582 mmol), triethylamine (118 mg, 1.16 mmol), CDI (188.63 mg, 1.16 mmol) and 1-[(3S)-3-aminopyrrolidin-1-yl]ethanone (149 mg, 1.16 mmol) in dichloromethane (21 mL). The residue was purified by reverse phase flash column chromatography eluting with acetonitrile (10-50%) in water (0.1% trifluoroacetic acid) to yield the title compound as an orange solid (230 mg, 59%).

### 3-[(3S)-1-acetylpyrrolidin-3-yl]-1-(5-ethynyl-2-{[4-(4-methylpiperazin-1-yl)phenyl]amino}pyrido[2,3-d]pyrimidin-7-yl)urea

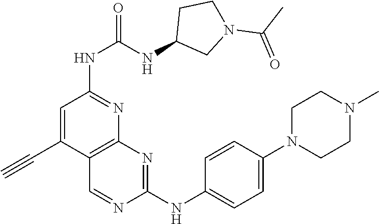 [SMILES: C#Cc1cc(NC(=O)N[C@H]2CCN(C(C)=O)C2)nc2nc(Nc3ccc(N4CCN(C)CC4)cc3)ncc12]

General Procedure 1 was applied to 3-[(3S)-1-acetylpyrrolidin-3-yl]-1-(2-{[4-(4-methylpiperazin-1-yl)phenyl]amino}-5-[2-(triisopropylsilyl)ethynyl]pyrido[2,3-d]pyrimidin-7-yl)urea (230 mg, 0.343 mmol) and potassium fluoride (398 mg, 6.86 mmol) in THF (2.5 mL), DMF (2.5 mL) and water (230 uL). The reaction mixture was stirred for 2 h at 60° C. The residue was purified by reverse phase flash column chromatography eluting with acetonitrile (10-50%) in water (0.1% trifluoroacetic acid) to yield the title compound as an orange solid (50.3 mg, 28%).

(ES, m/z): [M+H]⁺=514.26

¹H-NMR (400 MHz, DMSO-d₆): 5 10.13 (d, J=10.2 Hz, 1H), 9.96 (s, 1H), 9.57 (s, 1H), 9.06 (s, 1H), 7.80 (d, J=7.8 Hz, 2H), 7.20 (s, 1H), 7.04-6.89 (m, 2H), 5.05 (s, 1H), 4.45-4.22 (m, 1H), 3.76 (dd, J=10.5, 5.6 Hz, 1H), 3.66 (t, J=7.0 Hz, 2H), 3.58 (dt, J=13.0, 6.6 Hz, 3H), 3.11 (t, J=4.9 Hz, 4H), 2.47 (t, J=4.9 Hz, 4H), 2.23 (s, 4H), 1.93 (d, J=10.4 Hz, 4H).

### Example 54

### 3-(1-acetylazetidin-3-yl)-1-(2-{[4-(4-methylpiperazin-1-yl)phenyl]amino}-5-[2-(triisopropylsilyl)ethynyl]pyrido[2,3-d]pyrimidin-7-yl)urea

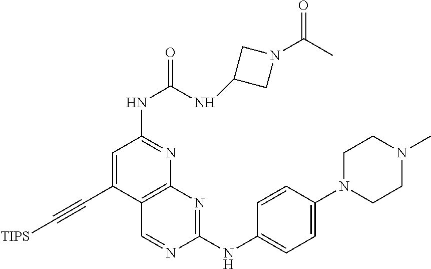

General Procedure 7 was applied to N²-[4-(4-methylpiperazin-1-yl)phenyl]-5-[2-(triisopropylsilyl)ethynyl]pyrido[2,3-d]pyrimidine-2,7-diamine (300 mg, 0.582 mmol), triethylamine (162 uL, 1.16 mmol), CDI (189 mg, 1.16 mmol) and 1-(3-aminoazetidin 1-yl)ethanone (132.79 mg, 1.16 mmol) in dichloromethane (20 mL). The residue was purified by reverse phase flash column chromatography eluting with acetonitrile (10-50%) in water (0.1% trifluoroacetic acid) to yield the title compound as a yellow solid (230 mg, 60%).

### 3-[(3R)-1-acetylpyrrolidin-3-yl]-1-(5-ethynyl-2-{[4-(4-methylpiperazin-1-yl)phenyl]amino}pyrido[2,3-d]pyrimidin-7-yl)urea

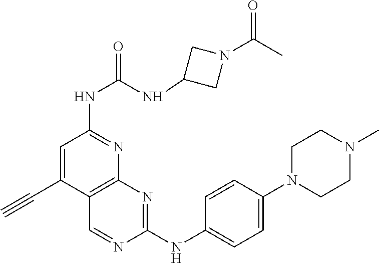 [SMILES: C#Cc1cc(NC(=O)NC2CN(C(C)=O)C2)nc2nc(Nc3ccc(N4CCN(C)CC4)cc3)ncc12]

General Procedure 1 was applied to 3-[(3R)-1-acetylpyrrolidin-3-yl]-1-(2-{[4-(4-methylpiperazin-1-yl)phenyl]amino}-5-[2-(triisopropylsilyl)ethynyl]pyrido[2,3-d]pyrimidin-7-yl)urea (230 mg, 0.343 mmol) and potassium fluoride (199 mg, 3.43 mmol) in THF (2.5 mL), DMF (2.5 mL) and water (230 uL). The reaction mixutre was stirred for 2 h at 60° C. The residue was purified by reverse phase flash column chromatography eluting with acetonitrile (10-50%) in water (0.1% trifluoroacetic acid) to yield the title compound as an orange solid (50.0 mg, 28%).

(ES, m/z): [M+H]⁺=500.24

¹H-NMR (400 MHz, DMSO-d₆) δ 10.26 (s, 1H), 9.99 (s, 1H), 9.76 (s, 1H), 9.08 (s, 1H), 7.82 (s, 2H), 7.24 (s, 1H), 6.93 (d, J=8.7 Hz, 2H), 5.06 (s, 1H), 4.49 (dt, J=22.5, 7.4 Hz, 2H), 4.22 (t, J=8.7 Hz, 1H), 4.02 (dd, J=8.5, 4.7 Hz, 1H), 3.78 (dd, J=10.0, 5.1 Hz, 1H), 3.12 (t, J=5.0 Hz, 5H), 2.53 (s, 4H), 2.28 (s, 3H), 1.80 (s, 3H), 1.23 (s, 1H).

### Example 55

### 1-(2-{[4-(4-methylpiperazin-1-yl)phenyl]amino}-5-[2-(triisopropylsilyl)ethynyl]pyrido[2,3-d]pyrimidin-7-yl)-3-(oxetan-3-ylmethyl)urea

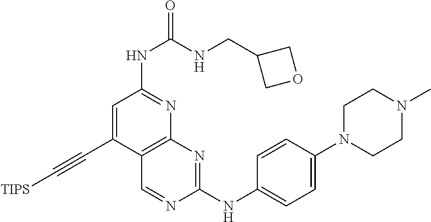

General Procedure 7 was applied to N²-[4-(4-methylpiperazin-1-yl)phenyl]-5-[2-(triisopropylsilyl)ethynyl]pyrido[2,3-d]pyrimidine-2,7-diamine (300 mg, 0.582 mmol), triethylamine (118 mg, 1.16 mmol), CDI (189 mg, 1.16 mmol) and 1-(oxetan-3-yl)methanamine (101 mg, 1.16 mmol) in dichloromethane (15 mL). The residue was purified by flash column chromatography eluting with methanol (3%) in dichloromethane to afford the title compound as an orange solid (248 mg, 68%).

(ES, m/z): [M+H]⁺=629.3

### 1-(5-ethynyl-2-{[4-(4-methylpiperazin-1-yl)phenyl]amino}pyrido[2,3-d]pyrimidin-7-yl)-3-(oxetan-3-ylmethyl)urea

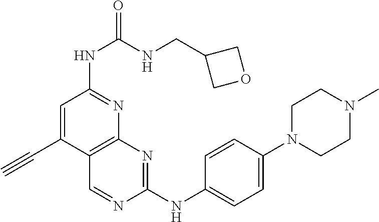 [SMILES: C#Cc1cc(NC(=O)NCC2COC2)nc2nc(Nc3ccc(N4CCN(C)CC4)cc3)ncc12]

General Procedure 1 was applied to 1-(2-{[4-(4-methylpiperazin-1-yl)phenyl]amino}-5-[2-(triisopropylsilyl)ethynyl]pyrido[2,3-d]pyrimidin-7-yl)-3-(oxetan-3-ylmethyl)urea (240 mg, 0.382 mmol) and potassium fluoride (222 mg, 3.82 mmol) in DMF (3 mL), THF (3 mL) and water (0.5 mL). The reaction mixture was stirred for 2 h at 60° C. The residue was purified by reverse phase flash column chromatography eluting with acetonitrile (20-30%) in water (0.4% formic acid) to afford the title compound as a yellow solid (79.0 mg, 42%).

(ES, m/z): [M+H]⁺=473.2

¹H NMR (400 MHz, DMSO-d₆) δ 10.14 (s, 1H), 9.95 (s, 1H), 9.24 (s, 1H), 9.06 (s, 1H), 7.79 (s, 2H), 7.24 (s, 1H), 6.97-6.80 (m, 2H), 5.04 (s, 1H), 4.71 (dd, J=7.9, 6.0 Hz, 2H), 4.43 (t, J=6.1 Hz, 2H), 3.54 (t, J=6.1 Hz, 2H), 3.21 (t, J=6.8 Hz, 1H), 3.08 (t, J=5.0 Hz, 4H), 2.47 (s, 4H), 2.24 (s, 3H).

### Example 56

### N-(2-{[4-(4-methylperazin-1-yl)phenyl]amino}-5-[2-(triisopropylsilyl)ethynyl]pyrido[2,3-d]pyrimidin-7-yl)pyrrolidine-1-carboxamide

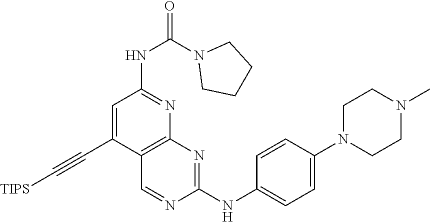

General Procedure 7 was applied to N²-[4-(4-methylpiperazin-1-yl)phenyl]-5-[2-(triisopropylsilyl)ethynyl]pyrido[2,3-d]pyrimidine-2,7-diamine (300 mg, 0.582 mmol), triethylamine (162 uL, 1.16 mmol), CDI (189 mg, 1.16 mmol) and pyrrolidine (95.5 uL, 1.16 mmol) in dichloromethane (21 mL). The residue was purified by reverse phase flash column chromatography eluting with acetonitrile (10-50%) in water (0.1% trifluoroacetic acid) to yield the title compound as an orange solid (300 mg, 84%).

### N-(5-ethynyl-2-{[4-(4-methylpiperazin-1-yl)phenyl]amino}pyrido[2,3-d]pyrimidin-7-yl)pyrrolidine-1-carboxamide

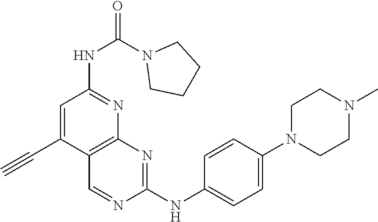 [SMILES: C#Cc1cc(NC(=O)N2CCCC2)nc2nc(Nc3ccc(N4CCN(C)CC4)cc3)ncc12]

General Procedure 1 was applied to N-(2-{[4-(4-methylpiperazin-1-yl)phenyl]amino}-5-[2-(triisopropylsilyl)ethynyl]pyrido[2,3-d]pyrimidin-7-yl)pyrrolidine-1-carboxamide (300 mg, 0.489 mmol) and potassium fluoride (284 mg, 4.89 mmol) in THE (3 mL), DMF (3 mL) and water (300 uL). The reaction mixture was stirred for 2 h at 60° C. The residue was purified by reverse phase flash column chromatography eluting with acetonitrile (10-50%) in water (0.1% trifluoroacetic acid) to yield the title compound as an orange red solid (70.3 mg, 31%).

(ES, m/z): [M+H]⁺=457.24

¹H-NMR (400 MHz, DMSO-d₆) δ 9.80 (d, J=67.1 Hz, 2H), 9.10 (s, 1H), 8.03 (s, 1H), 7.78 (d, J=8.5 Hz, 2H), 6.96-6.85 (m, 2H), 5.00 (s, 1H), 4.28-3.54 (m, 3H), 3.09 (t, J=4.9 Hz, 4H), 2.49-2.41 (m, 4H), 2.23 (s, 3H), 1.85 (s, 5H).

### Example 57

### tert-butyl N-(1-methanesulfonylazetidin-3-yl)carbamate

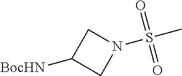 [SMILES: CC(C)(C)OC(=O)NC1CN(S(C)(=O)=O)C1]

To a stirred solution of 3-[(tert-butoxycarbonyl)amino]azetidin-1-ium chloride (2.00 g, 9.584 mmol, 1.0 eq.) and triethylamine (3.60 g, 35.6 mmol, 3.7 eq.) in dichloromethane (10 mL) was added methanesulfonyl chloride (2.00 g, 17.4 mmol, 1.8 eq.) dropwise at 0° C. The resulting mixture was stirred for 2 h at room temperature then concentrated under reduced pressure. The residue was purified by reverse phase flash column chromatography eluting with acetonitrile (40-50%) in water (0.1% formic acid) to afford the title compound as a white solid (886 mg, 37%).

### 1-methanesulfonylazetidin-3-amine

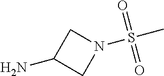

To a solution of tert-butyl N-(1-methanesulfonylazetidin-3-yl)carbamate (50.0 mg, 0.200 mmol, 1.0 eq.) in dichloromethane (2 mL) was added trifluoroacetic acid (0.4 mL) at room temperature and stirred overnight. The mixture was concentrated under reduced pressure to give the crude title compound (30.0 mg, 100%), which was used without further purification.

### 3-(1-methanesulfonylazetidin-3-yl)-1-(2-{[4-(4-methylpiperazin-1-yl)phenyl]amino}-5-[2-(triisopropylsilyl)ethynyl]pyrido[2,3-d]pyrimidin-7-yl)urea

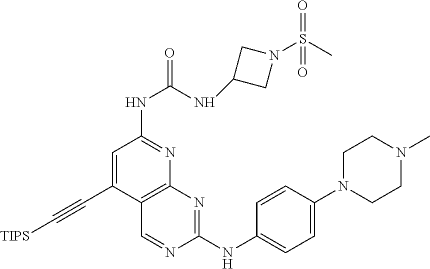

General Procedure 7 was applied to N²-[4-(4-methylpiperazin-1-yl)phenyl]-5-[2-(triisopropylsilyl)ethynyl]pyrido[2,3-d]pyrimidine-2,7-diamine (300 mg, 0.582 mmol), triethylamine (118 mg, 1.16 mmol), CDI (189 mg, 1.16 mmol) and 1-methanesulfonylazetidin-3-amine (175 mg, 1.16 mmol) in dichloromethane (20 mL). The residue was purified by reverse phase flash column chromatography eluting with acetonitrile (10-50%) in water (0.1% trifluoroacetic acid) to yield the title compound as an orange solid (300 mg, 75%).

### 1-(5-ethynyl-2-{[4-(4-methylpiperazin-1-yl)phenyl]amino}pyrido[2,3-d]pyrimidin-7-yl)-3-(1-methanesulfonylazetidin-3-yl)urea

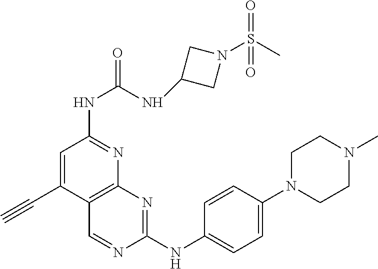 [SMILES: C#Cc1cc(NC(=O)NC2CN(S(C)(=O)=O)C2)nc2nc(Nc3ccc(N4CCN(C)CC4)cc3)ncc12]

General Procedure 1 was applied to 3-(1-methanesulfonylazetidin-3-yl)-1-(2-{[4-(4-methylpiperazin-1-yl)phenyl]amino}-5-[2-(triisopropylsilyl)ethynyl]pyrido[2,3-d]pyrimidin-7-yl)urea (280 mg, 0.405 mmol) and potassium fluoride (235 mg, 4.05 mmol) in THE (3 mL), DMF (3 mL) and water (280 uL). The reaction mixture was stirred for 2 h at 60° C. The residue was purified by reverse phase flash column chromatography eluting with acetonitrile (10-50%) in water (0.1% trifluoroacetic acid) to yield the title compound as an orange solid (132 mg, 61%).

(ES, m/z): [M+H]⁺=536.21

¹H-NMR (400 MHz, DMSO-d₆) δ 10.29 (s, 1H), 10.01 (s, 1H), 9.72 (s, 1H), 9.08 (s, 1H), 7.85 (s, 2H), 7.26 (s, 1H), 6.99-6.90 (m, 2H), 5.07 (s, 1H), 4.60-4.51 (m, 1H), 4.22 (t, J=8.1 Hz, 2H), 3.85 (dd, J=8.5, 5.9 Hz, 2H), 3.10 (t, J=4.9 Hz, 4H), 3.06 (s, 3H), 2.25 (s, 3H).

### Example 58

### N-(2-{[4-(4-methylpiperazin-1-yl)phenyl]amino}-5-[2-(triisopropylsilyl)ethynyl]pyrido[2,3-d]pyrimidin-7-yl)-1,3-dihydroisoindole-2-carboxamide

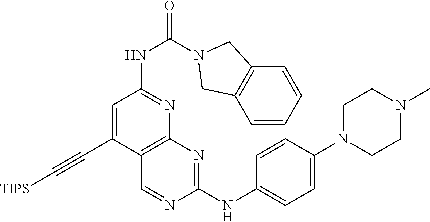

General Procedure 7 was applied to N²-[4-(4-methylpiperazin-1-yl)phenyl]-5-[2-(triisopropylsilyl)ethynyl]pyrido[2,3-d]pyrimidine-2,7-diamine (300 mg, 0.582 mmol), triethylamine (118 mg, 1.16 mmol), CDI (189 mg, 1.16 mmol) and isoindoline (139 mg, 1.16 mmol) in dichloromethane (20 mL). The residue was purified by reverse phase flash column chromatography eluting with acetonitrile (10-50%) in water (0.1% trifluoroacetic acid) to yield the title compound as an orange solid (300 mg, 78%).

### N-(5-ethynyl-2-{[4-(4-methylpiperazin-1-yl)phenyl]amino}pyrido[2,3-d]pyrimidin-7-yl)-1,3-dihydroisoindole-2-carboxamide

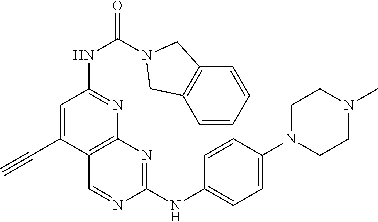 [SMILES: C#Cc1cc(NC(=O)N2Cc3ccccc3C2)nc2nc(Nc3ccc(N4CCN(C)CC4)cc3)ncc12]

General Procedure 1 was applied to N-(2-{[4-(4-methylpiperazin-1-yl)phenyl]amino}-5-[2-(triisopropylsilyl)ethynyl]pyrido[2,3-d]pyrimidin-7-yl)-1,3-dihydroisoindole-2-carboxamide (280 mg, 0.424 mmol) and potassium fluoride (246 mg, 4.24 mmol) in THF (3 mL), DMF (3 mL) and water (280 uL). The reaction mixture was stirred for 2 h at 60° C. The residue was purified by reverse phase flash column chromatography eluting with acetonitrile (10-50%) in water (0.1% trifluoroacetic acid) to yield the title compound as an orange solid (40.3 mg, 19%).

(ES, m/z): [M+H]⁺=505.24

¹H-NMR (400 MHz, DMSO-d₆, ppm): 5 9.96 (d, J=29.8 Hz, 2H), 9.12 (s, 1H), 8.05 (s, 1H), 7.79 (d, J=8.5 Hz, 2H), 7.39-7.26 (m, 4H), 6.98-6.88 (m, 2H), 5.03 (s, 5H), 3.10 (t, J=4.9 Hz, 4H), 2.47 (t, J=5.0 Hz, 4H), 2.23 (s, 3H).

### Example 59

### 3-(1H-imidazol-2-yl)-1-(2-{[4-(4-methylpiperazin-1-yl)phenyl]amino}-5-[2-(triisopropylsilyl)ethynyl]pyrido[2,3-d]pyrimidin-7-yl)urea

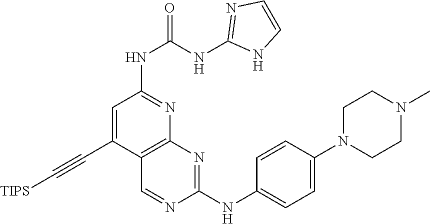

General Procedure 7 was applied to N²-[4-(4-methylpiperazin-1-yl)phenyl]-5-[2-(triisopropylsilyl)ethynyl]pyrido[2,3-d]pyrimidine-2,7-diamine (800 mg, 1.55 mmol), triethylamine (314 mg, 3.10 mmol), CDI (503 mg, 3.10 mmol) and 1H-imidazol-2-amine (258 mg, 3.10 mmol) in dichloromethane (50 mL). The residue was purified by reverse phase flash column chromatography eluting with acetonitrile (10-50%) in water (0.1% trifluoroacetic acid) to yield the title compound as an orange solid (350 mg, 36%).

### 1-(5-ethynyl-2-{[4-(4-methylpiperazin-1-yl)phenyl]amino}pyrido[2,3-d]pyrimidin-7-yl)-3-(1H-imidazol-2-yl)urea

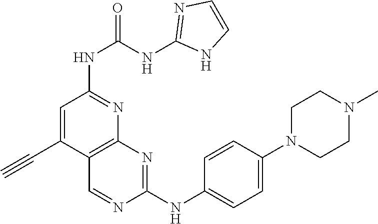 [SMILES: C#Cc1cc(NC(=O)Nc2ncc[nH]2)nc2nc(Nc3ccc(N4CCN(C)CC4)cc3)ncc12]

General Procedure 1 was applied to 3-(1H-imidazol-2-yl)-1-(2-{[4-(4-methylpiperazin-1-yl)phenyl]amino}-5-[2-(triisopropylsilyl)ethynyl]pyrido[2,3-d]pyrimidin-7-yl)urea (350 mg, 0.560 mmol) and potassium fluoride (325 mg, 5.60 mmol) in THF (4 mL), DMF (4 mL) and water (350 uL). The reaction mixture was stirred for 2 h at 60° C. The residue was purified by reverse phase flash column chromatography eluting with acetonitrile (10-50%) in water (0.1% trifluoroacetic acid) to yield the title compound as an orange solid (36.0 mg, 14%).

(ES, m/z): [M+H]⁺=469.21

¹H-NMR (400 MHz, DMSO-d₆) δ 11.48 (s, 2H), 10.25 (d, J=168.7 Hz, 2H), 9.13 (s, 1H), 7.74 (s, 3H), 6.93 (d, J=8.6 Hz, 2H), 6.78 (s, 2H), 5.08 (s, 1H), 3.13-3.06 (m, 4H), 2.47 (t, J=4.8 Hz, 4H), 2.23 (s, 3H).

### Example 60

### 1-(2-(methylthio)-5-((triisopropylsilyl)ethynyl)pyrido[2,3-d]pyrimidin-7-yl)-3-(1-(trifluoromethyl)cyclopentyl)urea

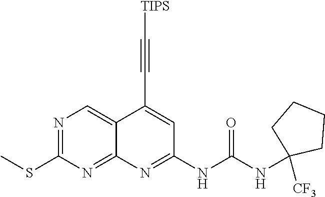

General Procedure 7 was applied to 2-(methylsulfanyl)-5-[2-(triisopropylsilyl)ethynyl]pyrido[2,3-d]pyrimidin-7-amine (100 mg, 0.271 mmol), triethylamine (40.0 uL, 0.400 mmol), CDI (65.0 mg, 0.400 mmol) and 1-trifluoromethyl-1-cyclopentylamine (62.0 mg, 0.542 mmol) in dichloromethane (1 mL). The residue was purified by flash column chromatography eluting with ethyl acetate (20%) in 40-60 petroleum ether to afford the title compound as a yellow solid (100 mg, 88%).

(ES, m/z): [M+H]⁺=552.4

¹H NMR (500 MHz, CDCl₃): δ_{H}=1.07 (3H, s), 1.08 (18H, s), 1.75 (2H, m), 1.88 (2H, m), 2.15 (2H, m), 2.40 (2H, m), 2.58 (3H, s), 7.09 (1H, br s, NH), 9.19 (1H, s), 10.76 (1H, br s, NH), 10.85 (1H, s).

### 1-(2-((4-(4-methylpiperazin-1-yl)phenyl)amino)-5-((triisopropylsilyl)ethynyl)pyrido[2,3-D]pyrimidin-7-yl)-3-(1-(trifluoromethyl)cyclopentyl)urea

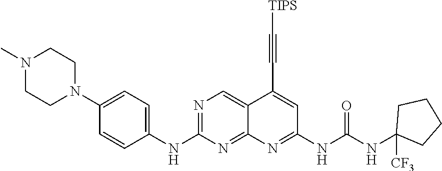

General Procedure 2 was applied to 1-(2-(methylthio)-5-((triisopropylsilyl)ethynyl)pyrido[2,3-d]pyrimidin-7-yl)-3-(1-(trifluoromethyl)cyclopentyl)urea (130 mg, 0.241 mmol) and m-CPBA (123 mg, 0.712 mmol) in dichloromethane (5 mL) to yield the title compound as a brown solid (135 mg, 100%).

General Procedure 3 was applied to 1-(2-(methanesulfonyl)-5-((triisopropylsilyl)ethynyl)pyrido[2,3-d]pyrimidin-7-yl)-3-(1-(trifluoromethyl)cyclopentyl)urea (135 mg, 0.241 mmol), 4-(4-methylpiperazin-1-yl) aniline (69.0 mg, 0.363 mmol) and trifluoroacetic acid (29.0 uL, 0.363 mmol) in acetonitrile (2.4 mL). The resulting mixture was stirred at 85° C. overnight. The residue was purified by flash column chromatography eluting with methanol (10%) in dichloromethane to yield the title compound as a red solid (55.0 mg, 33%).

(ES, m/z): [M+H]⁺=695.5

¹H NMR (500 MHz, CDCl₃): δ_{H}=1.16 (3H, s), 1.17 (18H, s), 1.83 (3H, s), 1.97 (2H, brs), 2.23 (2H, m), 2.45 (3H, s), 2.49 (2H, br s), 2.73 (4H, br s), 3.24 (4H, br s), 6.93 (2H, br s), 7.71 (2H, m), 9.17 (1H, s), 10.46 (1H, s).

### 1-(5-ethynyl-2-((4-(4-methylpiperazin-1-yl)phenyl)amino)pyrido[2,3-d]pyrimidin-7-yl)-3-(1-(trifluoromethyl)cyclopentyl)urea

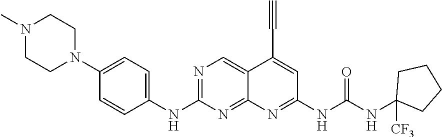 [SMILES: C#Cc1cc(NC(=O)NC2(C(F)(F)F)CCCC2)nc2nc(Nc3ccc(N4CCN(C)CC4)cc3)ncc12]

General Procedure 1 was applied to 1-(2-((4-(4-methylpiperazin-1-yl)phenyl)amino)-5-((triisopropylsilyl)ethynyl)pyrido[2,3-d]pyrimidin-7-yl)-3-(1-(trifluoromethyl)cyclopentyl)urea (55.0 mg, 80.0 μmol) and potassium fluoride (92.0 mg, 51.58 mmol) in DMF (1.6 mL). The resulting mixture was stirred at room temperature overnight. The residue was purified by flash column chromatography eluting with methanol (10-50%) in dichloromethane to afford the title compound as a yellow solid.

(ES, m/z): [M+H]⁺=539.4

¹H NMR (500 MHz, CDCl₃): δ_{H}=1.84 (2H, br s), 1.97 (2H, br s), 2.22 (4H, m), 2.40 (3H, s), 2.47 (2H, m), 2.65 (4H, m), 3.21 (4H, m), 3.62 (1H, s), 6.93 (2H, d, J=7.6 Hz), 7.72 (2H, br s), 9.15 (1H, s), 10.04 (1H, br s).

### Example 61

### 1-(3-methyloxetan-3-yl)-3-(2-(methylthio)-5-((triisopropylsilyl)ethynyl)pyrido[2,3-d]pyrimidin-7-yl)urea

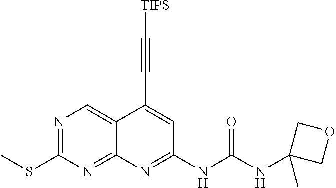

General Procedure 7 was applied to 2-(methylsulfanyl)-5-[2-(triisopropylsilyl)ethynyl]pyrido[2,3-d]pyrimidin-7-amine (330 mg, 0.891 mmol), triethylamine (182 uL, 1.34 mmol), CDI (214 mg, 1.34 mmol) and 3-methyloxetan-3-amine (155 mg, 1.78 mmol) in dichloromethane (3 mL). The residue was purified by flash column chromatography eluting with ethyl acetate (20%) in 40-60 petroleum ether to afford the title compound as a white solid (400 mg, 93%).

(ES, m/z): [M+H]⁺=486.3

¹H NMR (500 MHz, CDCl₃): δ_{H}=1.17 (3H, s), 1.18 (18H, s), 1.85 (3H, s), 2.69 (3H, s), 4.61 (2H, d, J=6.0 Hz), 4.95 (2H, br m), 7.18 (1H, br s, NH), 9.31 (1H, s), 10.45 (1H, br s, NH), 10.96 (1H, s).

### 1-(3-methyloxetan-3-yl)-3-(2-((4-(4-methylpiperazin-1-yl)phenyl)amino)-5-((triisopropylsilyl)ethynyl)pyrido[2,3-d]pyrimidin-7-yl)urea

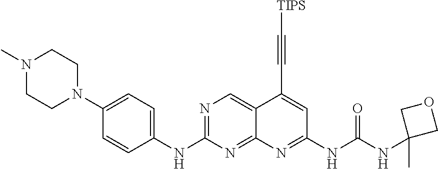

General Procedure 2 was applied to 1-(3-methyloxetan-3-yl)-3-(2-(methylthio)-5-((triisopropylsilyl)ethynyl)pyrido[2,3-d]pyrimidin-7-yl)urea (400 mg, 0.821 mmol) and m-CPBA (412 mg, 2.46 mmol) in dichloromethane (16 mL) to yield the title compound as a brown solid (416 mg, 100%).

General Procedure 3 was applied to 1-(3-methyloxetan-3-yl)-3-(2-(methylsulfonyl)-5-((triisopropylsilyl)ethynyl)pyrido[2,3-d]pyrimidin-7-yl)urea (416 mg, 0.821 mmol), 4-(4-methylpiperazin-1-yl) aniline (235 mg, 1.23 mmol) and trifluoroacetic acid (98.0 uL, 1.23 mmol) in acetonitrile (8.2 mL). The resulting mixture was stirred at 85° C. overnight. The residue was purified by flash column chromatography eluting with methanol (10%) in dichloromethane to yield the title compound as a red solid (60.0 mg, 11%).

(ES, m/z): [M+H]⁺=629.5

¹H NMR (500 MHz, CDCl₃): δ_{H}=1.06 (3H, s), 1.07 (18H, s), 1.63 (2H, s), 2.29 (3H, m), 2.53 (4H, br t), 3.10 (4H, br t), 4.43 (1H, s), 4.82 (1H, s), 6.83 (2H, d, J=8.6 Hz), 7.50 (2H, m), 9.10 (1H, s).

### 1-(5-ethynyl-2-((4-(4-methylpiperazin-1-yl)phenyl)amino)pyrido[2,3-d]pyrimidin-7-yl)-3-(3-methyloxetan-3-yl)urea

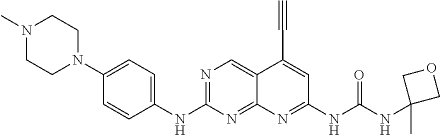 [SMILES: C#Cc1cc(NC(=O)NC2(C)COC2)nc2nc(Nc3ccc(N4CCN(C)CC4)cc3)ncc12]

General Procedure 1 was applied to 1-(3-methyloxetan-3-yl)-3-(2-((4-(4-methylpiperazin-1-yl)phenyl)amino)-5-((triisopropylsilyl)ethynyl)pyrido[2,3-d]pyrimidin-7-yl)urea (60.0 mg, 90.0 μmol) and potassium fluoride (111 mg, 1.90 mmol) in DMF (1.8 mL). The resulting mixture was stirred at room temperature overnight. The residue was purified by flash column chromatography eluting with methanol (10-50%) in dichloromethane to afford the title compound as a red solid.

(ES, m/z): [M+H]⁺=473.3

¹H NMR (500 MHz, DMSO): δ_{H}=1.65 (3H, s), 2.22 (3H, s), 2.46 (4H, t, J=4.8 Hz), 3.08 (4H, t, J=4.8 Hz), 4.44 (2H, d, J=6.0 Hz), 4.72 (2H, d, J=6.0 Hz), 5.07 (1H, s), 6.91 (2H, d, J=9.1 Hz), 7.29 (1H, s), 7.81 (2H, br s), 9.07 (1H, s), 9.96 (1H, br s), 10.16 (1H, br s, NH).

### Example 62

### 1-(3-methyltetrahydrofuran-3-yl)-3-(2-(methylthio)-5-((triisopropylsilyl)ethynyl)pyrido[2,3-d]pyrimidin-7-yl)urea

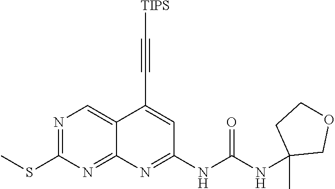

General Procedure 7 was applied to 2-(methylsulfanyl)-5-[2-(triisopropylsilyl)ethynyl]pyrido[2,3-d]pyrimidin-7-amine (100 mg, 0.271 mmol), triethylamine (40.0 uL, 0.400 mmol), CDI (65.0 mg, 0.400 mmol) and aminotetrahydrofuran (41.0 mg, 0.400 mmol) in dichloromethane (1 mL). The residue was purified by flash column chromatography eluting with ethyl acetate (20%) in 40-60 petroleum ether to afford the title compound as a yellow solid (100 mg, 88%).

(ES, m/z): [M+H]⁺=500.4

¹H NMR (500 MHz, CD₃OD): δ_{H}=1.15 (3H, s), 1.16 (18H, s), 1.54 (3H, s), 2.03 (1H, m), 2.41 (1H, m), 2.61 (3H, s), 3.67 (1H, d, J=8.8 Hz), 3.88-4.00 (2H, m), 4.07 (1H, d, J=8.8 Hz), 7.25 (1H, s), 9.17 (1H, s).

### 1-(2-((4-(4-methylpiperazin-1-yl)phenyl)amino)-5-((triisopropylsilyl)ethynyl)pyrido[2,3-d]pyrimidin-7-yl)-3-(3-methyltetrahydrofuran-3-yl)urea

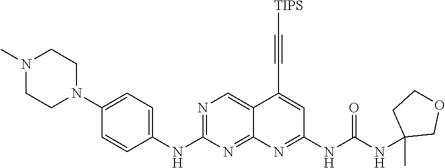

General Procedure 2 was applied to 1-(3-methyltetrahydrofuran-3-yl)-3-(2-(methylthio)-5-((triisopropylsilyl)ethynyl)pyrido[2,3-d]pyrimidin-7-yl)urea (120 mg, 0.241 mmol) and m-CPBA (124 mg, 0.723 mmol) in dichloromethane (5 mL) to yield the title compound as a brown solid (123 mg, 100%).

General Procedure 3 was applied to 1-(3-methyltetrahydrofuran-3-yl)-3-(2-(methylsulfonyl)-5-((triisopropylsilyl)ethynyl)pyrido[2,3-d]pyrimidin-7-yl)urea (123 mg, 0.241 mmol), 4-(4-methylpiperazin-1-yl) aniline (69.0 mg, 0.362 mmol) and trifluoroacetic acid (29.0 uL, 0.362 mmol) in acetonitrile (2.4 mL). The resulting mixture was stirred at 85° C. overnight. The residue was purified by flash column chromatography eluting with methanol (10%) in dichloromethane to yield the title compound as a red solid (90.0 mg, 58%).

(ES, m/z): [M+H]⁺=643.5

¹H NMR (500 MHz, DMSO): δ_{H}=1.14 (3H, s), 1.16 (18H, s), 1.51 (3H, s), 2.01 (2H, m), 2.23 (3H, s), 2.27 (2H, m), 2.47 (4H, t, J=4.5 Hz), 3.08 (4H, t, J=4.5 Hz), 3.60 (1H, d, J=8.7 Hz), 3.90 (3H, m), 6.89 (2H, d, J=9.0 Hz), 7.24 (1H, s), 7.81 (2H, br s), 9.00 (1H, s), 9.94 (1H, br s, NH).

### 1-(5-ethynyl-2-((4-(4-methylpiperazin-1-yl)phenyl)amino)pyrido[2,3-d]pyrimidin-7-yl)-3-(3-methyloxetan-3-yl)urea

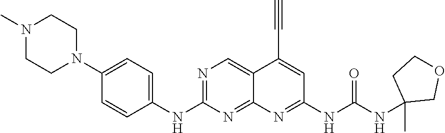 [SMILES: C#Cc1cc(NC(=O)NC2(C)CCOC2)nc2nc(Nc3ccc(N4CCN(C)CC4)cc3)ncc12]

General Procedure 1 was applied to 1-(2-((4-(4-methylpiperazin-1-yl)phenyl)amino)-5-((triisopropylsilyl)ethynyl)pyrido[2,3-d]pyrimidin-7-yl)-3-(3-methyltetrahydrofuran-3-yl)urea (90.0 mg, 141 μmol) and potassium fluoride (163 mg, 2.80 mmol) in DMF (3 mL). The resulting mixture was stirred at room temperature overnight. The residue was purified by flash column chromatography eluting with methanol (10-50%) in dichloromethane to afford the title compound as a red solid.

(ES, m/z): [M+H]⁺=487.3

¹H NMR (500 MHz, DMSO): δ_{H}=1.52 (3H, s), 2.01 (1H, m), 2.26 (2H, m), 2.31 (3H, s), 2.58 (4H, t, J=5.0 Hz), 3.11 (4H, t, J=5.0 Hz), 3.60 (1H, d, J=8.6 Hz), 3.92 (4H, m), 5.08 (1H, s, alkyne CH), 7.18 (1H, s), 7.84 (2H, br s), 9.05 (1H, s), 10.02 (1H, br s, NH), 10.06 (1H, br s, NH).

### Example 63

### 1-(2-((3-((cyclobutylmethyl)amino)-4-(4-methylpiperazin-1-yl)phenyl)amino)-5-((triisopropylsilyl)ethynyl)pyrido[2,3-d]pyrimidin-7-yl)-3-cyclopentylurea

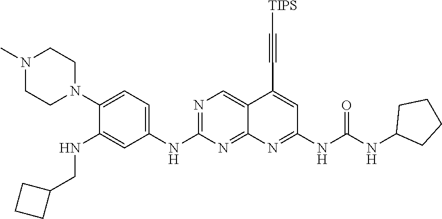

General Procedure 3 was applied to 1-cyclopentyl-3-{2-methanesulfonyl-5-[2-(triisopropylsilyl)ethynyl]pyrido[2,3-d]pyrimidin-7-yl}urea (103 mg, 0.200 mmol), 4-(4-methylpiperazin-1-yl) aniline (82.0 mg, 0.302 mmol) and trifluoroacetic acid (23.0 uL, 0.302 mmol) in acetonitrile (2 mL). The resulting mixture was stirred at 85° C. overnight. The residue was purified by flash column chromatography eluting with methanol (10%) in dichloromethane to yield the title compound as a red solid (50.0 mg, 35%).

(ES, m/z): [M+H]⁺=710.6

¹H NMR (500 MHz, CDCl₃): δ_{H}=1.16 (3H, s), 1.17 (18H, s), 1.64 (4H, m), 1.78 (4H, m), 1.87-2.05 (5H, m), 2.11 (3H, s), 2.42 (3H, s), 2.65 (2H, m), 2.93 (5H, m), 3.10 (2H, d, J=7.1 Hz), 4.30 (1H, sl), 6.68 (1H, br s), 6.99 (1H, d, J=8.5 Hz), 7.28 (1H, s), 9.19 (1H, s), 10.0 (1H, br s).

### 1-(2-((3-((cyclobutylmethyl)amino)-4-(4-methylpiperazin-1-yl)phenyl)amino)-5-ethynyl)pyrido[2,3-d]pyrimidin-7-yl)-3-cyclopentylurea

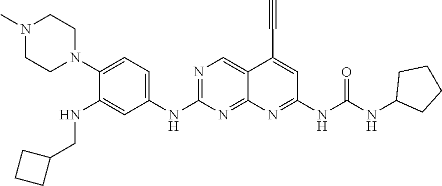 [SMILES: C#Cc1cc(NC(=O)NC2CCCC2)nc2nc(Nc3ccc(N4CCN(C)CC4)c(NCC4CCC4)c3)ncc12]

General Procedure 1 was applied to 1-(2-((3-((cyclobutylmethyl)amino)-4-(4-methylpiperazin-1-yl)phenyl)amino)-5-((triisopropylsilyl)ethynyl)pyrido[2,3-d]pyrimidin-7-yl)-3-cyclopentylurea (50.0 mg, 70.0 μmol) and potassium fluoride (82.0 mg, 1.41 mmol) in DMF (1.4 mL). The resulting mixture was stirred at room temperature overnight. The residue was purified by flash column chromatography eluting with methanol (10-50%) in dichloromethane to afford the title compound as a red solid.

(ES, m/z): [M+H]⁺=554.4

¹H NMR (500 MHz, DMSO): δ_{H}=1.23 (1H, s), 1.53 (2H, m), 1.66 (2H, m), 1.72-1.82 (4H, m), 1.83-1.95 (4H, m), 2.00-2.09 (2H, m), 2.24 (4H, s), 2.63 (2H, m), 2.76 (4H, br t), 3.07 (4H, t, J=6.4 Hz), 4.12 (1H, m), 4.74 (1H, t, J=5.7 Hz), 5.05 (1H, s, alkyne CH), 6.86 (2H, m), 7.16 (1H, s), 9.06 (1H, s), 9.48 (1H, br s), 9.82 (1H, s), 10.00 (1H, br s, NH).

### Example 64

### tert-butyl N-(3-methyl-2-{[2-(methylsulfanyl)-5-[2-(triisopropylsilyl) ethynyl]pyrido[2,3-d]pyrimidin-7-yl]amino}butyl)carbamate

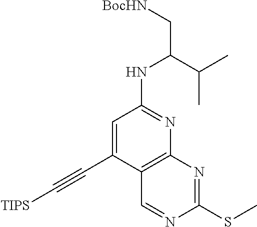

A solution of 7-chloro-2-(methylsulfanyl)-5-[2-(triisopropylsilyl)ethynyl]pyrido[2,3-d]pyrimidine (800 mg, 2.04 mmol, 1.0 eq.), tert-butyl N-(2-amino-3-methylbutyl)carbamate (454 mg, 2.25 mmol, 1.1 eq.) and N,N-diisopropyl-N-ethylamine (0.710 mL, 4.08 mmol, 2.0 eq.) in dioxane (10 mL) was stirred for 18 h at 100° C.

The mixture was cooled to room temperature and concentrated under reduced pressure. The residue was purified by flash column chromatography eluting with ethyl acetate (6%) in 40-60 petroleum ether to afford the title compound as a light yellow solid (800 mg, 70%).

### tert-butyl N-[2-({2-methanesulfonyl-5-[2-(triisopropylsilyl)ethynyl]pyrido[2,3-d]pyrimidin-7-yl}amino)-3-methylbutyl]carbamate

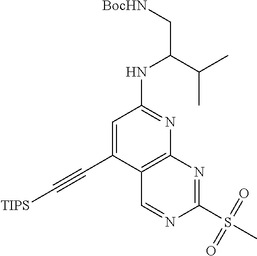

General Procedure 2 was applied to tert-butyl N-(3-methyl-2-{[2-(methylsulfanyl)-5-[2-(triisopropylsilyl)ethynyl]pyrido[2,3-d]pyrimidin-7-yl]amino}butyl)carbamate (750 mg, 1.34 mmol) and m-CPBA (232 mg, 1.34 mmol) in dichloromethane (10 mL) to yield the title compound as a light yellow solid (739 mg, 93%).

### tert-butyl N-{3-methyl-2-[(2-{[4-(4-methylpiperazin-1-yl)phenyl]amino}-5-[2-(triisopropylsilyl)ethynyl]pyrido[2,3-d]pyrimidin-7-yl)amino]butyl}carbamate

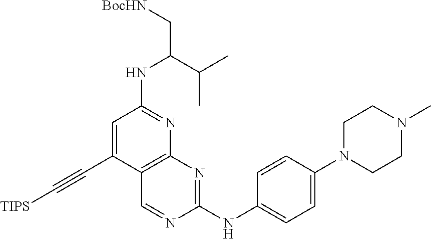

General Procedure 3 was applied to tert-butyl N-[2-({2-methanesulfonyl-5-[2-(triisopropylsilyl)ethynyl]pyrido[2,3-d]pyrimidin-7-yl}amino)-3-methylbutyl]carbamate (710 mg, 1.20 mmol), 4-(4-methylpiperazin-1-yl)aniline (276 mg, 1.45 mmol) and trifluoroacetic acid (0.180 mL, 2.41 mmol) in 2-butanol (8 mL). The reaction mixture was stirred for 1 h at 100° C. to yield the title compound as a red solid (820 mg, 97%).

### N¹-(1-amino-3-methylbutan-2-yl)-N²-[4-(4-methylpiperazin-1-yl)phenyl]-5-[2-(triisopropylsilyl) ethynyl]pyrido[2,3-d]pyrimidine-2,7-diamine

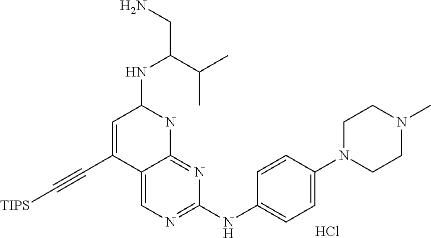

A solution of tert-butyl N-{3-methyl-2-[(2-{[4-(4-methylpiperazin-1-yl)phenyl]amino}-5-[2-(triisopropylsilyl)ethynyl]pyrido[2,3-d]pyrimidin-7-yl)amino]butyl}carbamate (810 mg, 1.16 mmol, 1.0 eq.) and hydrogen chloride in 1,4-dioxane (0.35 mL, 11.6 mmol, 10 eq.) in dichloromethane (10 mL) was stirred for 2 h at room temperature then concentrated under reduced pressure to yield the title compound as a red solid (572 mg, 82%). 5-isopropyl-1-(2-{[4-(4-methylpiperazin-1-yl)phenyl]amino}-5-[2-(triisopropylsilyl)ethynyl]pyrido[2,3-d]pyrimidin-7-yl)imidazolidin-2-one

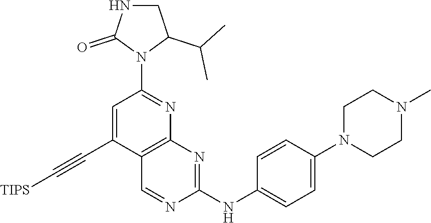

A solution of N⁷-(1-amino-3-methylbutan-2-yl)-N²-[4-(4-methylpiperazin-1-yl)phenyl]-5-[2-(triisopropylsilyl)ethynyl]pyrido[2,3-d]pyrimidine-2,7-diamine (300 mg, 0.499 mmol), triethylamine (202 mg, 1.99 mmol) and CDI (162 mg, 0.998 mmol) in THF (5 mL) was stirred for 2 h at room temperature. The resulting mixture was diluted with water (20 mL) and extracted with ethyl acetate (3×20 mL). The residue was purified by reverse phase flash column chromatography eluting with acetonitrile (10-80%) in water (0.1% formic acid) to yield the title compound as a red solid (142 mg, 45%).

### 1-(5-ethynyl-2-{[4-(4-methylpiperazin-1-yl)phenyl]amino}pyrido[2,3-d]pyrimidin-7-yl)-5-isopropylmidazolidin-2-one

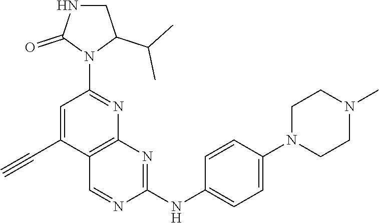 [SMILES: C#Cc1cc(N2C(=O)NCC2C(C)C)nc2nc(Nc3ccc(N4CCN(C)CC4)cc3)ncc12]

General Procedure 1 was applied to 5-isopropyl-1-(2-{[4-(4-methylpiperazin-1-yl)phenyl]amino}-5-[2-(triisopropylsilyl)ethynyl]pyrido[2,3-d]pyrimidin-7-yl)imidazolidin-2-one (180 mg, 0.287 mmol) and potassium fluoride (167 mg, 2.87 mmol) in THF (2 mL), DMF (2 mL) and water (0.5 mL). The reaction mixture was stirred for 1 h at 40° C. The residue was purified by reverse phase flash column chromatography eluting with acetonitrile (10-60%) in water (0.1% formic acid) to yield the title compound as a red solid (110 mg, 81%).

(ES, m/z): [M+H]⁺=471

¹H-NMR (300 MHz, DMSO-d₆) δ 9.92 (s, 1H), 9.12 (s, 1H), 8.41 (s, 1H), 7.70 (d, J=13.9 Hz, 3H), 6.94 (d, J=8.7 Hz, 2H), 5.00 (s, 1H), 4.84-4.69 (m, 1H), 3.47 (t, J=9.5 Hz, 1H), 3.26 (dd, J=9.6, 3.2 Hz, 1H), 3.17 (t, J=5.0 Hz, 4H), 2.76-2.57 (m, 5H), 2.39 (s, 3H), 0.93 (d, J=7.0 Hz, 3H), 0.77 (d, J=6.8 Hz, 3H).

### Example 65

### Cyclopentyl (2-(methylthio)-5-((triisopropylsilyl)ethynyl)pyrido[2,3-d]pyrimidin-7-yl)carbamate

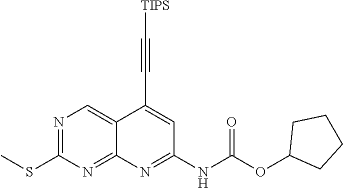

General Procedure 7 was applied to 2-(methylsulfanyl)-5-[2-(triisopropylsilyl)ethynyl]pyrido[2,3-d]pyrimidin-7-amine (100 mg, 0.271 mmol), triethylamine (40.0 uL, 0.400 mmol), CDI (65.0 mg, 0.400 mmol) and cyclopentylalcohol (46.5 mg, 0.540 mmol) in dichloromethane (1 mL). The residue was purified by flash column chromatography eluting with ethyl acetate (20%) in 40-60 petroleum ether to afford the title compound as a yellow solid (130 mg, 99%).

(ES, m/z): [M+H]⁺=485.4

¹H NMR (500 MHz, CDCl₃): δ_{H}=1.17 (3H, s), 1.19 (18H, s), 1.54-1.63 (2H, m), 1.63-1.72 (2H, m), 1.81-1.91 (2H, m), 1.92-2.01 (2H, m), 2.72 (3H, s), 5.028 (1H, m), 8.45 (1H, s), 9.36 (1H, s).

### Cyclopentyl (2-((4-(4-methylpiperazin-1-yl)phenyl)amino)-5-((triisopropylsilyl)ethynyl)pyrido[2,3-d]pyrimidin-7-yl)carbamate

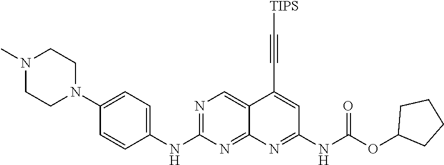

General Procedure 2 was applied to cyclopentyl (2-(methylthio)-5-((triisopropylsilyl)ethynyl)pyrido[2,3-d]pyrimidin-7-yl)carbamate (130 mg, 0.271 mmol) and m-CPBA (140 mg, 0.813 mmol) in dichloromethane (5 mL) to yield the title compound as a brown solid (140 mg, 100%).

General Procedure 3 was applied to cyclopentyl (2-(methylsulfonyl)-5-((triisopropylsilyl)ethynyl)pyrido[2,3-d]pyrimidin-7-yl)carbamate (140 mg, 0.271 mmol), 4-(4-methylpiperazin-1-yl) aniline (77.5 mg, 0.405 mmol) and trifluoroacetic acid (32.0 uL, 0.405 mmol) in acetonitrile (2.7 mL). The resulting mixture was stirred at 85° C. overnight. The residue was purified by flash column chromatography eluting with methanol (10%) in dichloromethane to yield the title compound as a red solid (60.0 mg, 35%).

(ES, m/z): [M+H]⁺=628.6

¹H NMR (500 MHz, CDCl₃): δ_{H}=1.08 (3H, s), 1.09 (18H, s), 1.47-1.59 (2H, m), 1.59-1.67 (2H, m), 1.67-1.76 (2H, m), 1.78-1.88 (2H, m), 2.45 (3H, s), 2.77 (4H, br t), 3.18 (4H, br t), 5.15 (1H, m), 6.82 (2H, d, J=8.8 Hz), 7.56 (2H, br d), 7.72 (2H, br s), 8.06 (1H, s), 8.21 (1H, sl), 9.20 (1H, s).

### cyclopentyl (5-ethynyl-2-((4-(4-methylpiperazin-1-yl)phenyl)amino)pyrido[2,3-d]pyrimidin-7-yl)carbamate

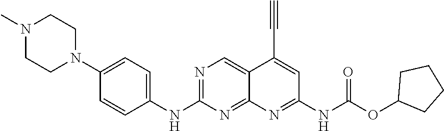 [SMILES: C#Cc1cc(NC(=O)OC2CCCC2)nc2nc(Nc3ccc(N4CCN(C)CC4)cc3)ncc12]

General Procedure 1 was applied to cyclopentyl (2-((4-(4-methylpiperazin-1-yl)phenyl)amino)-5-((triisopropylsilyl)ethynyl)pyrido[2,3-d]pyrimidin-7-yl)carbamate (60.0 mg, 95.0 μmol) and potassium fluoride (111 mg, 1.91 mmol) in DMF (2 mL). The resulting mixture was stirred at room temperature overnight. The residue was purified by flash column chromatography eluting with methanol (10-50%) in dichloromethane to afford the title compound as a red solid (30.0 mg, 67%).

(ES, m/z): [M+H]⁺=472.3

¹H NMR (500 MHz, CDCl₃): δ_{H}=1.49-1.60 (2H, m), 1.60-1.77 (4H, m), 1.78-1.89 (2H, m), 2.29 (3H, s), 2.54 (4H, br t), 3.10 (4H, br t), 3.58 (1H, m), 5.15 (1H, m), 6.82 (2H, d, J=8.9 Hz), 7.54 (2H, br s), 8.12 (1H, s), 9.35 (1H, br s, NH).

### Example 66

### Cyclohexyl (2-(methylthio)-5-((triisopropylsilyl)ethynyl)pyrido[2,3-d]pyrimidin-7-yl)carbamate

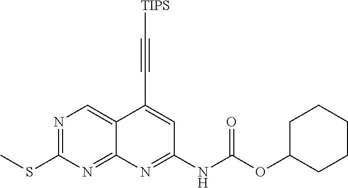

General Procedure 7 was applied to 2-(methylsulfanyl)-5-[2-(triisopropylsilyl)ethynyl]pyrido[2,3-d]pyrimidin-7-amine (50.0 mg, 0.131 mmol), triethylamine (28.0 uL, 0.195 mmol), CDI (32.5 mg, 0.195 mmol) and cyclohexanol (426.5 mg, 0.260 mmol) in dichloromethane (0.5 mL). The residue was purified by flash column chromatography eluting with ethyl acetate (20%) in 40-60 petroleum ether to afford the title compound as a yellow solid (50.0 mg, 77%).

(ES, m/z): [M+H]⁺=499.4

¹H NMR (500 MHz, CDC₃): δ_{H}=1.07 (3H, s), 1.09 (18H, s), 1.18-1.26 (1H, m), 1.26-1.38 (2H, m), 1.39-1.53 (3H, m), 1.62-1.72 (2H, m), 1.81-1.90 (2H, m), 2.61 (3H, s), 4.74 (1H, m), 7.91 (1H, br s), 8.28 (1H, s), 9.28 (1H, s).

### cyclohexyl (2-((4-(4-methylpiperazin-1-yl)henyl)amino)-5-((triisopropylsilyl)ethynyl)pyrido[2,3-d]pyrimidin-7-yl)carbamate

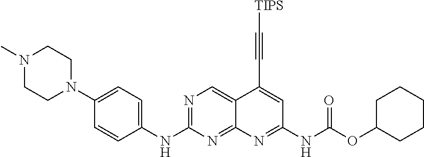

General Procedure 2 was applied to cyclohexyl (2-(methylthio)-5-((triisopropylsilyl)ethynyl)pyrido[2,3-d]pyrimidin-7-yl)carbamate (330 mg, 0.661 mmol) and m-CPBA (343 mg, 1.98 mmol) in dichloromethane (13 mL) to yield the title compound as a brown solid (330 mg, 99%).

General Procedure 3 was applied to cyclohexyl (2-(methylsulfonyl)-5-((triisopropylsilyl)ethynyl)pyrido[2,3-d]pyrimidin-7-yl)carbamate (330 mg, 0.651 mmol), 4-(4-methylpiperazin-1-yl) aniline (127 mg, 0.977 mmol) and trifluoroacetic acid (80.0 uL, 0.977 mmol) in acetonitrile (6.0 mL). The resulting mixture was stirred at 85° C. overnight. The residue was purified by flash column chromatography eluting with methanol (10%) in dichloromethane to yield the title compound as a red solid (90.0 mg, 22%).

¹H NMR (500 MHz, CDCl₃): δ_{H}=1.15 (3H, s), 1.17 (18H, s), 1.24-1.33 (2H, m), 1.34-1.47 (2H, m), 1.47-1.60 (3H, m), 1.69-1.80 (2H, m), 1.86-1.99 (2H, m), 2.48 (3H, s), 2.79 (4H, br t), 3.27 (4H, br t), 4.80 (1H, m), 6.93 (2H, d, J=9.0 Hz), 7.65 (2H, m), 8.14 (1H, s), 9.26 (1H, s)

### cyclohexyl (5-ethynyl-2-((4-(4-methylpiperazin-1-yl)phenyl)amino)pyrido[2,3-d]pyrimidin-7-yl)carbamate

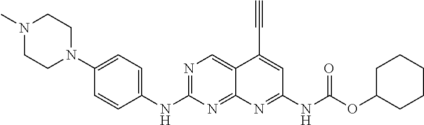 [SMILES: C#Cc1cc(NC(=O)OC2CCCCC2)nc2nc(Nc3ccc(N4CCN(C)CC4)cc3)ncc12]

General Procedure 1 was applied to cyclohexyl (2-((4-(4-methylpiperazin-1-yl)phenyl)amino)-5-((triisopropylsilyl)ethynyl)pyrido[2,3-d]pyrimidin-7-yl)carbamate (90 mg, 135 μmol) and potassium fluoride (163 mg, 2.79 mmol) in DMF (2.3 mL). The resulting mixture was stirred at room temperature overnight. The residue was purified by flash column chromatography eluting with methanol (10-50%) in dichloromethane to afford the title compound as a red solid (40.0 mg, 57%).

(ES, m/z): [M+H]⁺=486.4

¹H NMR (500 MHz, CDCl₃): δ_{H}=1.20-1.33 (2H, m), 1.35-1.45 (2H, m), 1.46-1.61 (3H, m), 1.69-1; 79 (2H, m), 1.89-1.99 (2H, m), 2.37 (3H, m), 2.61 (4H, br t), 3.17 (4H, br t), 3.66 (1H, s), 4.80 (1H, m), 6.90 (2H, d, J=8.9 Hz), 7.62 (2H, br s), 8.20 (1H, s), 8.52 (1H, br s, NH), 9.25 (1H, s).

### Example 67

### benzyl (2-(methylthio)-5-((triisopropylsilyl)ethynyl)pyrido[2,3-d]pyrimidin-7-yl)carbamate

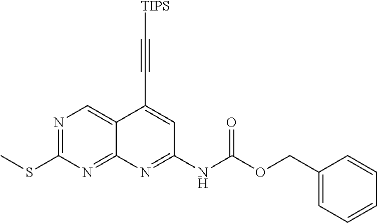

General Procedure 7 was applied to 2-(methylsulfanyl)-5-[2-(triisopropylsilyl)ethynyl]pyrido[2,3-d]pyrimidin-7-amine (160 mg, 0.431 mmol), triethylamine (92.0 uL, 0.642 mmol), CDI (104 mg, 0.642 mmol) and benzylalcohol (93.0 mg, 0.862 mmol) in dichloromethane (1.7 mL). The residue was purified by flash column chromatography eluting with ethyl acetate (20%) in 40-60 petroleum ether to afford the title compound as a yellow solid (200 mg, 93%).

(ES, m/z): [M+H]⁺=507.4

¹H NMR (500 MHz, CDCl₃): δ_{H}=1.17 (3H, s), 1.19 (18H, s), 2.70 (3H, s), 5.28 (2H, s), 7.34-7.44 (5H, m), 8.16 (1H, br s), 8.37 (1H, s), 9.38 (1H, s).

### benzyl (2-((4-(4-methylpiperazin-1-yl)phenyl)amino)-5-((triisopropylsilyl)ethynyl)pyrido[2,3-d]pyrimidin-7-yl)carbamate

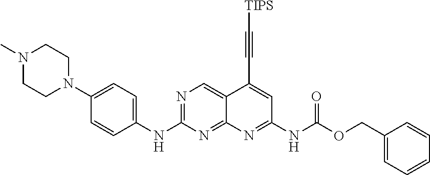

General Procedure 2 was applied to benzyl (2-(methylthio)-5-((triisopropylsilyl)ethynyl)pyrido[2,3-d]pyrimidin-7-yl)carbamate (200 mg, 0.401 mmol) and m-CPBA (207 mg, 1.20 mmol) in dichloromethane (8 mL) to yield the title compound as a brown solid (200 mg, 100%).

General Procedure 3 was applied to benzyl (2-(methylsulfonyl)-5-((triisopropylsilyl)ethynyl)pyrido[2,3-d]pyrimidin-7-yl)carbamate (200 mg, 0.401 mmol), 4-(4-methylpiperazin-1-yl) aniline (115 mg, 0.602 mmol) and trifluoroacetic acid (48.0 uL, 0.602 mmol) in acetonitrile (4.0 mL). The resulting mixture was stirred at 85° C. overnight. The residue was purified by flash column chromatography eluting with methanol (10%) in dichloromethane to yield the title compound as a red solid (110 mg, 42%).

(ES, m/z): [M+H]⁺=650.6

¹H NMR (500 MHz, CDCl₃): δ_{H}=1.15 (3H, s), 1.16 (18H, s), 2.40 (3H, s), 2.68 (4H, br t), 3.19 (4H, br t), 5.21 (2H, s), 6.85 (2H, d, J=8.8 Hz), 7.29-7.39 (5H, m), 7.59 (2H, m), 8.12 (1H, s), 9.26 (1H, s).

### benzyl (5-ethynyl-2-((4-(4-methylpiperazin-1-yl)phenyl)amino)pyrido[2,3-d]pyrimidin-7-yl)carbamate

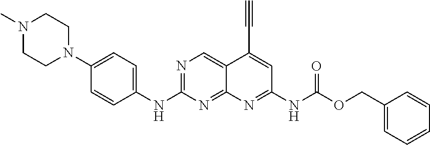 [SMILES: C#Cc1cc(NC(=O)OCc2ccccc2)nc2nc(Nc3ccc(N4CCN(C)CC4)cc3)ncc12]

General Procedure 1 was applied to cyclohexyl (2-((4-(4-methylpiperazin-1-yl)phenyl)amino)-5-((triisopropylsilyl)ethynyl)pyrido[2,3-d]pyrimidin-7-yl)carbamate (110 mg, 170 μmol) and potassium fluoride (197 mg, 3.39 mmol) in DMF (3.4 mL). The resulting mixture was stirred at room temperature overnight. The residue was purified by flash column chromatography eluting with methanol (10-50%) in dichloromethane to afford the title compound as a red solid (25.0 mg, 30%).

(ES, m/z): [M+H]⁺=494.4

¹H NMR (500 MHz, CDCl₃): δ_{H}=2.44 (3H, s), 2.70 (4H, br t), 3.24 (4H, br t), 3.66 (1H, s), 5.27 (2H, s), 6.95 (2H, d, J=8.9 Hz), 7.34-7.45 (5H, m), 7.56 (1H, br s, NH), 7.63 (2H, br s), 8.17 (1H, br s, NH), 8.21 (1H, s), 9.27 (1H, s).

### Example 68

### 4-isoprooyl-3-[2-(methylsulfanyl)-5-[2-(triisoproovisilyl)ethynyl]pyrido[2,3-d]pyrimidin-7-yl]-1,3-oxazolidin-2-one

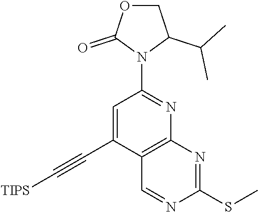

A solution of 7-chloro-2-(methylsulfanyl)-5-[2-(triisopropylsilyl)ethynyl]pyrido[2,3-d]pyrimidine (1.00 g, 2.55 mmol, 1.0 eq.), 4-isopropyl-1,3-oxazolidin-2-one (428 mg, 3.32 mmol, 1.3 eq.), tris(dibenzyldeneacetone)dipalladium(0) (73.3 mg, 0.128 mmol, 0.05 eq.), XantPhos (147 mg, 0.255 mmol, 0.1 eq.) and potassium carbonate (705 mg, 5.10 mmol, 2.0 eq.) in dioxane (30 mL) was stirred for 1 h at 100° C. The mixture was cooled to room temperature then diluted with ethyl acetate (30 mL). The resulting mixture was filtered, washing with ethyl acetate (2×10 mL), the filtrate was concentrated under reduced pressure. The residue was purified by flash column chromatography eluting with ethyl acetate (8%) in 40-60 petroleum ether to afford the title compound as a yellow solid (768 mg, 62%).

### 4-isopropyl-3-{2-methanesulfonyl-5-[2-(triisopropylsilyl)ethynyl]pyrido[2,3-d]pyrimidin-7-yl}-1,3-oxazolidin-2-one

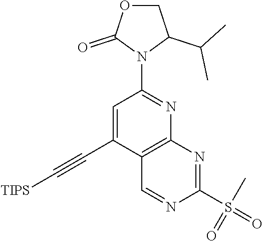

General Procedure 2 was applied to 4-isopropyl-3-[2-(methylsulfanyl)-5-[2-(triisopropylsilyl)ethynyl]pyrido[2,3-d]pyrimidin-7-yl]-1,3-oxazolidin-2-one (370 mg, 0.763 mmol) and m-CPBA (290 mg, 1.68 mmol) in dichloromethane (6 mL) to yield the title compound as a light yellow solid (372 mg, 94%).

### 4-isopropyl-3-(2-{[4-(4-methylpiperazin-1-yl)phenyl]amino}-5-[2-(triisopropylsilyl)ethynyl]pyrido[2,3-d]pyrimidin-7-yl)-1,3-oxazolidin-2-one

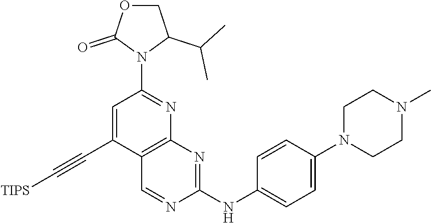

General Procedure 3 was applied to 4-isopropyl-3-{2-methanesulfonyl-5-[2-(triisopropylsilyl)ethynyl]pyrido[2,3-d]pyrimidin-7-yl}-1,3-oxazolidin-2-one (365 mg, 0.706 mmol), 4-(4-methylpiperazin-1-yl)aniline (203 mg, 1.06 mmol) and trifluoroacetic acid (0.10 mL, 1.41 mmol) in 2-butanol (5 mL). The reaction mixture was stirred for 1 h at 100° C. The residue was purified by reverse phase flash column chromatography eluting with acetonitrile (40-100%) in water (0.1% formic acid) to yield the title compound as a red solid (204 mg, 46%).

### 3-(5-ethynyl-2-{[4-(4-methylpiperazin-1-yl)phenyl]amino}pyrido[2,3-d]pyrimidin-7-yl)-4-isopropyl-1.3-oxazolidin-2-one

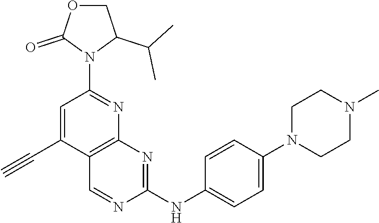 [SMILES: C#Cc1cc(N2C(=O)OCC2C(C)C)nc2nc(Nc3ccc(N4CCN(C)CC4)cc3)ncc12]

General Procedure 1 was applied to 4-isopropyl-3-(2-{[4-(4-methylpiperazin-1-yl)phenyl]amino}-5-[2-(triisopropylsilyl)ethynyl]pyrido[2,3-d]pyrimidin-7-yl)-1,3-oxazolidin-2-one (190 mg, 0.303 mmol) and potassium fluoride (176 mg, 3.03 mmol) in THF (2 mL), water (0.5 mL) and DMF (2 mL). The reaction mixture was stirred for 1 h at 60° C. The residue was purified by reverse phase flash column chromatography eluting with acetonitrile (10-60%) in water (0.1% formic acid) to yield the title compound as a red solid (62.0 mg, 43%).

(ES, m/z): [M+H]⁺=472.

¹H NMR (400 MHz, DMSO-d₆) δ 10.04 (s, 1H), 9.21 (s, 1H), 8.17 (s, 1H), 7.70 (s, 2H), 6.93 (d, J=8.8 Hz, 2H), 5.10 (s, 1H), 4.92 (dd, J=7.7, 3.9 Hz, 1H), 4.52-4.38 (m, 2H), 3.11 (t, J=4.9 Hz, 4H), 2.68-2.53 (m, 1H), 2.48 (s, 4H), 2.25 (s, 3H), 0.94 (d, J=7.0 Hz, 3H), 0.79 (d, J=6.9 Hz, 3H).

### Example 69

### (4R)-4-isopropyl-3-[2-(methylsulfanyl)-5-[2-(triisopropylsilyl)ethynyl]pyrido[2,3-d]pyrimidin-7-yl]-1,3-oxazolidin-2-one

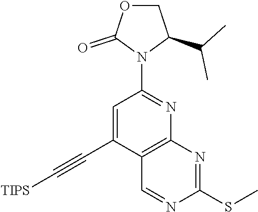

A solution of 7-chloro-2-(methylsulfanyl)-5-[2-(triisopropylsilyl)ethynyl]pyrido[2,3-d]pyrimidine (1.00 g, 2.55 mmol, 1.0 eq.), (4R)-4-isopropyl-1,3-oxazolidin-2-one (400 mg, 3.06 mmol, 1.2 eq.), tris(dibenzyldeneacetone)dipalladium(0) (120 mg, 0.128 mmol, 0.05 eq.), XantPhos (150 mg, 0.255 mmol, 0.1 eq.) and potassium carbonate (710 mg, 5.10 mmol, 2.0 eq.) in dioxane (15 mL) was stirred for 1.5 h at 100° C.

The mixture was cooled to room temperature and concentrated under reduced pressure. The residue was purified by flash column chromatography eluting with ethyl acetate (9%) in 40-60 petroleum ether to afford the title compound as a red oil (689 mg, 56%).

### (4R)-4-isopropyl-3-{2-methanesulfonyl-5-[2-(triisopropylsilyl)ethynyl]pyrido[2,3-d]pyrimidin-7-yl}-1,3-oxazolidin-2-one

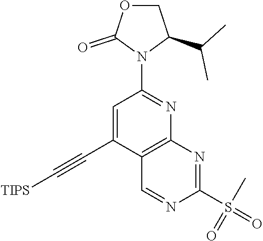

General Procedure 2 was applied to (4R)-4-isopropyl-3-[2-(methylsulfanyl)-5-[2-(triisopropylsilyl)ethynyl]pyrido[2,3-d]pyrimidin-7-yl]-1,3-oxazolidin-2-one (680 mg, 1.40 mmol) and m-CPBA (532 mg, 3.09 mmol) in dichloromethane (10 mL) to yield the title compound as a red oil (672 mg, 93%).

### (4R)-4-isopropyl-3-(2-{[4-(4-methylpiperazin-1-yl)phenyl]amino}-5-[2-(triisopropylsilyl) ethynyl]pyrido[2,3-d]pyrimidin-7-yl)-1,3-oxazolidin-2-one

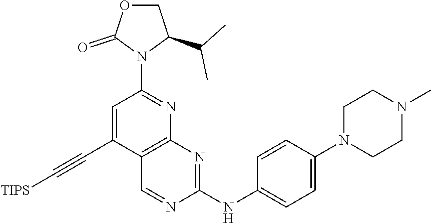

General Procedure 3 was applied to (4R)-4-isopropyl-3-{2-methanesulfonyl-5-[2-(triisopropylsilyl)ethynyl]pyrido[2,3-d]pyrimidin-7-yl}-1,3-oxazolidin-2-one (650 mg, 1.26 mmol), 4-(4-methylpiperazin-1-yl)aniline (289 mg, 1.51 mmol) and trifluoroacetic acid (0.19 mL, 2.52 mmol) in 2-butanol (5 mL) was stirred for 2 h at 80° C. The residue was purified by reverse phase flash column chromatography eluting with acetonitrile (10-70%) in water (0.1% formic acid) to yield the title compound as a red solid (230 mg, 29%).

### (4R)-3-(5-ethynyl-2-{[4-(4-methylpiperazin-1-yl)phenyl]amino}pyrido[2,3-d]pyrimidin-7-yl)-4-isopropyl-1,3-oxazolidin-2-one

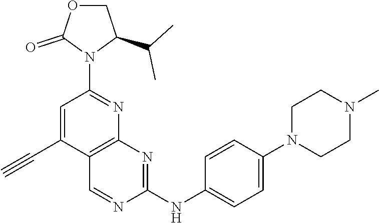 [SMILES: C#Cc1cc(N2C(=O)OC[C@H]2C(C)C)nc2nc(Nc3ccc(N4CCN(C)CC4)cc3)ncc12]

General Procedure 1 was applied (4R)-4-isopropyl-3-(2-{[4-(4-methylpiperazin-1-yl)phenyl]amino}-5-[2-(triisopropylsilyl) ethynyl]pyrido[2,3-d]pyrimidin-7-yl)-1,3-oxazolidin-2-one (230 mg, 0.366 mmol) and potassium fluoride (213 mg, 3.66 mmol) in THF (2 mL), DMF (2 mL) and water (0.5 mL). The reaction mixture was stirred for 1 h at 60° C. The residue was purified by reverse phase flash column chromatography eluting with acetonitrile (10-50%) in water (0.1% formic acid) to yield the title compound as a red solid (86.0 mg, 50%).

(ES, m/z): [M+H]⁺=472

¹H NMR (300 MHz, DMSO-d₆) δ 10.05 (s, 1H), 9.21 (s, 1H), 8.17 (s, 1H), 7.70 (s, 2H), 7.00-6.86 (m, 2H), 5.11 (s, 1H), 4.91 (dd, J=7.7, 4.1 Hz, 1H), 4.53-4.40 (m, 2H), 3.10 (dd, J=6.4, 3.6 Hz, 4H), 2.57 (d, J=10.3 Hz, 1H), 2.47 (d, J=5.1 Hz, 4H), 2.24 (s, 3H), 0.94 (d, J=7.0 Hz, 3H), 0.80 (d, J=6.8 Hz, 3H).

### Example 70

### (4S)-4-isopropyl-3-[2-(methylsulfanyl)-5-[2-(triisopropylsilyl)ethynyl]pyrido[2,3-d]pyrimidin-7-yl]-1,3-oxazolidin-2-one

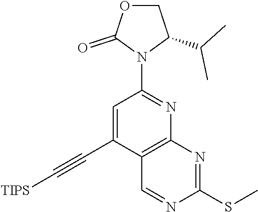

A solution of 7-chloro-2-(methylsulfanyl)-5-[2-(triisopropylsilyl)ethynyl]pyrido[2,3-d]pyrimidine (1.00 g, 2.55 mmol, 1.0 eq.), (4S)-4-isopropyl-1,3-oxazolidin-2-one (400 mg, 3.06 mmol, 1.2 eq.), tris(dibenzyldeneacetone)dipalladium(0) (120 mg, 0.128 mmol, 0.05 eq.), XantPhos (150 mg, 0.255 mmol, 0.1 eq.) and potassium carbonate (710 mg, 5.10 mmol, 2.0 eq.) in dioxane (15 mL) was stirred for 1.5 h at 100° C. The resulting mixture was concentrated under reduced pressure. The residue was purified by flash column chromatography eluting with ethyl acetate (8%) in 40-60 petroleum ether to afford the title compound as a red oil (683 mg, 55%).

### (4S)-4-isopropyl-3-{2-methanesulfonyl-5-[2-(triisopropylsilyl)ethynyl]pyrido[2,3-d]pyrimidin-7-yl}-1.3-oxazolidin-2-one

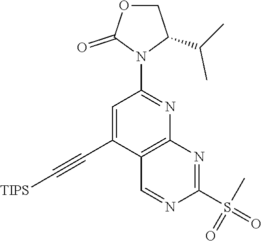

General Procedure 2 was applied to (4S)-4-isopropyl-3-[2-(methylsulfanyl)-5-[2-(triisopropylsilyl)ethynyl]pyrido[2,3-d]pyrimidin-7-yl]-1,3-oxazolidin-2-one (680 mg, 1.40 mmol) and m-CPBA (532 mg, 3.08 mmol) in dichloromethane (10 mL) to yield the title compound as a red oil (678 mg, 94%).

### (4S)-4-isopropyl-3-(2-{[4-(4-methylpiperazin-1-yl)phenyl]amino}-5-[2-(triisopropylsilyl)ethynyl]pyrido[2,3-d]pyrimidin-7-yl)-1,3-oxazolidin-2-one

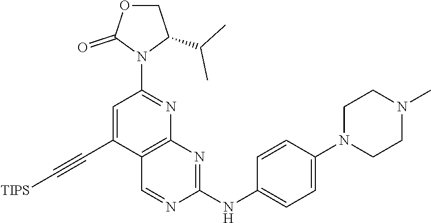

General Procedure 3 was applied to (4S)-4-isopropyl-3-{2-methanesulfonyl-5-[2-(triisopropylsilyl)ethynyl]pyrido [2,3-d]pyrimidin-7-yl}-1,3-oxazolidin-2-one (675 mg, 1.31 mmol), 4-(4-methylpiperazin-1-yl) aniline (300 mg, 1.57 mmol) and trifluoroacetic acid (0.19 mL, 2.61 mmol) in 2-butanol (5 mL). The reaction mixture was stirred for 2 h at 80° C. The residue was purified by reverse phase flash column chromatography eluting with acetonitrile (10-70%) in water (0.1% formic acid) to yield the title compound as a red solid (230 mg, 28%).

### (4S)-3-(5-ethynyl-2-{[4-(4-methylpiperazin-1-yl)phenyl]amino}pyrido[2,3-d]pyrimidin-7-yl)-4-isopropyl-1,3-oxazolidin-2-one

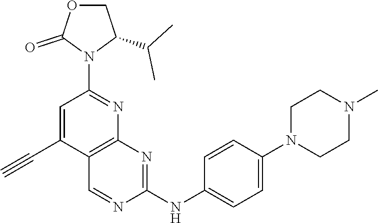 [SMILES: C#Cc1cc(N2C(=O)OC[C@@H]2C(C)C)nc2nc(Nc3ccc(N4CCN(C)CC4)cc3)ncc12]

General Procedure 1 was applied to (4S)-4-isopropyl-3-(2-{[4-(4-methylpiperazin-1-yl)phenyl]amino}-5-[2-(triisopropylsilyl)ethynyl]pyrido[2,3-d]pyrimidin-7-yl)-1,3-oxazolidin-2-one (230 mg, 0.366 mmol) and potassium fluoride (213 mg, 3.66 mmol) in THF (2 mL), DMF (2 mL) and water (0.5 mL). The reaction mixture was stirred for 1 h at 60° C. The residue was purified by reverse phase flash column chromatography eluting with acetonitrile (10-50%) in water (0.1% formic acid) to yield the title compound as a red solid (73.0 mg, 42%).

(ES, m/z): [M+H]⁺=472

¹H-NMR (300 MHz, DMSO-d₆, ppm) δ 10.05 (s, 1H), 9.21 (s, 1H), 8.17 (d, J=2.4 Hz, 1H), 7.70 (s, 2H), 7.00-6.88 (m, 2H), 5.11 (s, 1H), 5.00-4.81 (m, 1H), 4.54-4.40 (m, 2H), 3.11 (dd, J=6.4, 3.7 Hz, 4H), 2.64-2.54 (m, 1H), 2.48 (d, J=4.5 Hz, 4H), 2.25 (s, 3H), 0.95 (d, J=7.0 Hz, 3H), 0.80 (d, J=6.9 Hz, 3H).

### Example 71

### 3-[2-(methylsulfanyl)-5-[2-(triisopropylsilyl)ethynyl]pyrido[2,3-d]pyrimidin-7-yl]-1,3-oxazolidin-2-one

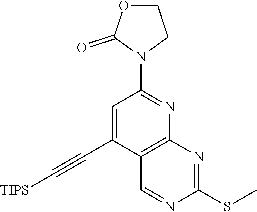

A solution of 7-chloro-2-(methylsulfanyl)-5-[2-(triisopropylsilyl)ethynyl]pyrido[2,3-d]pyrimidine (1.00 g, 2.55 mmol, 1.0 eq.), oxazolidinone (290 mg, 3.32 mmol, 1.3 eq.), tris(dibenzyldeneacetone)dipalladium(0) (120 mg, 0.128 mmol, 0.05 eq.), XantPhos (150 mg, 0.255 mmol, 0.1 eq.) and potassium carbonate (710 mg, 5.10 mmol, 2.0 eq.) in dioxane (10 mL) was stirred for 1 hat 100° C. The mixture was cooled to room temperature then diluted with water (50 mL) and extracted with ethyl acetate (2×30 mL). The combined organic layers were concentrated under reduced pressure. The residue was purified by flash column chromatography eluting with ethyl acetate (16%) in 40-60 petroleum ether to afford the title compound as a light yellow solid (850 mg, 75%).

### 3-{2-methanesulfonyl-5-[2-(triisopropylsilyl)ethynyl]pyrido[2,3-d]pyrimidin-7-yl}-1,3-oxazolidin-2-one

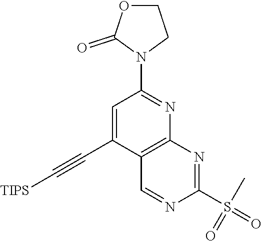

General Procedure 2 was applied to m-CPBA (618 mg, 3.58 mmol) and 3-[2-(methylsulfanyl)-5-[2-(triisopropylsilyl)ethynyl]pyrido[2,3-d]pyrimidin-7-yl]-1,3-oxazolidin-2-one (720 mg, 1.63 mmol) in dichloromethane (10 mL) to yield the title compound as a red solid (724 mg, 94%).

### 3-(2-{[4-(4-methylpiperazin-1-yl)phenyl]amino}-5-[2-(triisopropylsilyl)ethynyl]pyrido[2,3-d]pyrimidin-7-yl)-1,3-oxazolidin-2-one

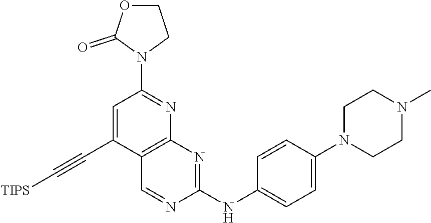

General Procedure 3 was applied to 3-{2-methanesulfonyl-5-[2-(triisopropylsilyl)ethynyl]pyrido[2,3-d]pyrimidin-7-yl}-1,3-oxazolidin-2-one (600 mg, 1.26 mmol), 4-(4-methylpiperazin-1-yl)aniline (290 mg, 1.52 mmol) and trifluoroacetic acid (0.190 mL, 2.53 mmol) in 2-butanol (10 mL). The reaction mixture was stirred for 1 h at 80° C. The residue was purified by reverse phase flash column chromatography eluting with acetonitrile (10-80%) in water (0.1% formic acid) to yield the title compound as a red solid (328 mg, 44%).

### 3-(5-ethynyl-2-{[4-(4-methylpiperazin-1-yl)phenyl]amino}pyrido[2,3-d]pyrimidin-7-yl)-1,3-oxazolidin-2-one

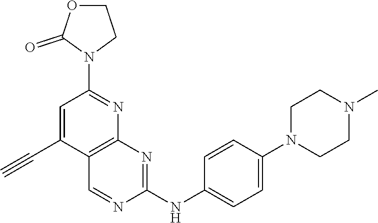 [SMILES: C#Cc1cc(N2CCOC2=O)nc2nc(Nc3ccc(N4CCN(C)CC4)cc3)ncc12]

General Procedure 1 was applied to 3-(2-{[4-(4-methylpiperazin-1-yl)phenyl]amino}-5-[2-(triisopropylsilyl)ethynyl]pyrido[2,3-d]pyrimidin-7-yl)-1,3-oxazolidin-2-one (150 mg, 0.256 mmol) and potassium fluoride (149 mg, 2.56 mmol) in THF (2 mL), DMF (2 mL) and water (0.5 mL). The reaction mixture was stirred for 1 h at 60° C. The residue was purified by reverse phase flash column chromatography eluting with acetonitrile (10-50%) in water (0.1% formic acid) to yield the title compound as a red solid (63.0 mg, 57%).

(ES, m/z): [M+H]⁺=444

¹H-NMR (300 MHz, DMSO-d₆) δ 10.00 (s, 1H), 9.21 (s, 1H), 8.17 (d, J=2.3 Hz, 1H), 7.73 (d, J=8.3 Hz, 2H), 7.01-6.87 (m, 2H), 5.10 (s, 1H), 4.51 (dd, J=8.7, 7.2 Hz, 2H), 4.38-4.21 (m, 2H), 3.11 (dd, J=6.3, 3.6 Hz, 4H), 2.48 (d, J=4.3 Hz, 4H), 2.25 (s, 3H)

### Example 72

### Ethyl 4-amino-2-(methylsulfanyl)pyrimidine-5-carboxylate

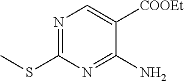

A solution of ethyl 4-chloro-2-(methylsulfanyl)pyrimidine-5-carboxylate (200 g, 859 mmol, 1.0 eq.) in ammonium hydroxide (400 mL) was treated with ethanol (600 mL) overnight at room temperature. The precipitated solids were collected by filtration, and washed with ethanol (3×10 mL). The resulting mixture was concentrated under reduced pressure to yield the title compound as a white solid (160 g, 87%).

### Ethyl 2-(methylsulfanyl)-4-propanamidopyrimidine-5-carboxylate

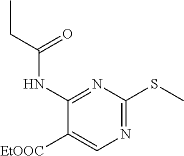 [SMILES: CCOC(=O)c1cnc(SC)nc1NC(=O)CC]

A solution of ethyl 4-amino-2-(methylsulfanyl)pyrimidine-5-carboxylate (19.0 g, 89.1 mmol, 1.0 eq.) and propionic anhydride (95 mL) was stirred overnight at 125° C. The mixture was allowed to cool to 15° C. The precipitated solids were collected by filtration, and washed with ethanol (3×30 mL) to yield the title compound as a pink solid (12.4 g, >100%). 5-hydroxy-6-methyl-2-(methylsulfanyl)-8H-pyrido[2,3-d]pyrimidin-7-one

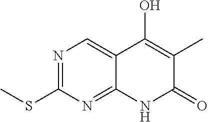

To a stirred solution of ethyl 2-(methylsulfanyl)-4-propanamidopyrimidine-5-carboxylate (10.3 g, 38.2 mmol, 1.0 eq.) in THF (62.0 mL) was added KHMDS (108 mL, 477 mmol, 12.5 eq.) dropwise at 0-10° C. over 10 minutes. The reaction was quenched with water (50 ml) at 0-10° C., concentrated under reduced pressure and then acidified to pH 3-4 with 4M aqueous hydrochloric acid. The precipitated solids were collected by filtration, and washed with water (3×5 mL). The resulting solids were dried under infrared light to yield the title compound as a white solid (7.25 g, 85%).

### 6-methyl-2-(methylsulfanyl)-7-oxo-8H-pyrido[2,3-d]pyrimidin-5-yl trifluoromethanesulfonate

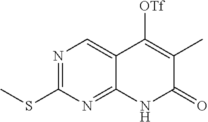

To a stirred solution of 5-hydroxy-6-methyl-2-(methylsulfanyl)-8H-pyrido[2,3-d]pyrimidin-7-one (7.00 g, 31.4 mmol, 1.0 eq.) in THE (70 mL) was added triethylamine (6.35 g, 62.7 mmol, 2.0 eq.). After stirring for 1 h at room temperature, 1,1,1-trifluoro-N-phenyl-N-trifluoromethanesulfonylmethanesulfonamide (16.8 g, 47.0 mmol, 1.5 eq.) was added and the mixture stirred for 40 minutes then quenched with water (3.0 mL) and extracted with dichloromethane (3×20 mL). The combined organic layers were washed with brine (3×5 mL) and concentrated under reduced pressure. The residue was purified by trituration with ethyl acetate (20 mL). The precipitated solids were collected by filtration, washing with ethyl acetate (3×5 mL) to yield the title compound as a white solid (5.03 g, 45%).

### 6-methyl-2-(methylsulfanyl)-5-[2-(triisoProvisilyl)ethynyl]-8H-pyrido[2,3-d]pyrimidin-7-one

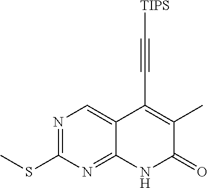

General Procedure 9 was applied The residue was purified by trituration with ethyl acetate (50 ml). The precipitated solids were collected by filtration, washing with ethyl acetate (3×10 mL) to yield the title compound.

A solution of 6-methyl-2-(methylsulfanyl)-7-oxo-8H-pyrido[2,3-d]pyrimidin-5-yl trifluoromethanesulfonate (4.00 g, 11.3 mmol, 1.0 eq.), triisopropylsilylacetylene (3.08 g, 16.9 mmol, 1.5 eq.), bis(triphenylphosphine)palladium(II) dichloride (395 mg, 0.563 mmol, 0.05 eq.) and copper(I) iodide (3.22 g, 16.9 mmol, 1.5 eq.) in ethyl acetate (40 mL, 0.25M) and triethylamine (6.84 g, 67.5 mmol, 6.0 eq.) was stirred at 60° C. overnight. The reaction mixture was concentrated under reduced pressure then dissolved in dichloromethane (100 mL) and washed with water (100 mL). The aqueous layer was extracted with dichloromethane (5×50 mL) and the combined organic layers were dried (MgSO₄) and concentrated under reduced pressure. The crude material was purified by flash column chromatography eluting with ethyl acetate (10-30%) in 40-60 petroleum ether to yield the title compound as a white solid (1.80 g, 41%).

### 7-Chloro-6-methyl-2-(methylsulfanyl)-5-[2-(triisopropylsilyl)ethynyl]pyrido[2,3-d]pyrimidine

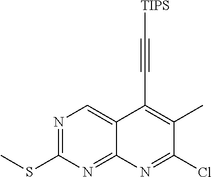

A solution of 6-methyl-2-(methylsulfanyl)-5-[2-(triisopropylsilyl)ethynyl]-8H-pyrido[2,3-d]pyrimidin-7-one (1.50 g, 3.87 mmol, 1.0 eq.) in phosphorus oxychloride (8.00 mL, 19.3 mmol, 5.0 eq.) was stirred for 2 hours at 80° C. The mixture was allowed to cool down to room temperature then quenched with water/ice. The aqueous layer was extracted with ethyl acetate (2×50 mL). The combined organic layers were dried (MgSO₄) and concentrated under reduced pressure to yield the title compound as a reddish brown oil (1.47 g, 93%).

### 6-Methyl-2-(methylsulfanyl)-5-[2-(triisopropylsilyl)ethynyl]pyrido[2,3-d]pyrimidin-7-amine

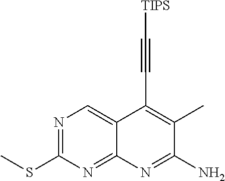

A solution of 7-chloro-6-methyl-2-(methylsulfanyl)-5-[2-(triisopropylsilyl)ethynyl]pyrido[2,3-d]pyrimidine (1.50 g, 3.69 mmol, 1.0 eq.) and aqueous ammonia (7.19 mL, 184 mmol, 50 eq.) in propan-2-ol (15 mL) was stirred for 2 hours at 100° C. The mixture was allowed to cool down to room temperature then diluted with water (50 mL) and extracted with ethyl acetate (2×50 mL). The combined organic layers were dried (MgSO₄) and concentrated under reduced pressure to yield the title compound as a reddish brown yellow solid (1.05 g, 73%).

### 2-Methanesulfonyl-6-methyl-5-[2-(triisopropylsilyl)ethynyl]pyrido[2,3-d]pyrimidin-7-amine

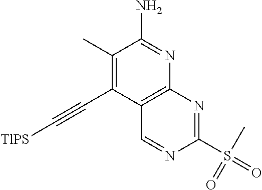

General Procedure 2 was applied to 6-methyl-2-(methylsulfanyl)-5-[2-(triisopropylsilyl)ethynyl]pyrido[2,3-d]pyrimidin-7-amine (1.05 g, 2.71 mmol) and m-CPBA (400 mg, 2.31 mmol) in dichloromethane (20 mL) to yield the title compound as a yellow solid (1.02 g, 89%).

### 6-Methyl-N²-[4-(4-methylpiperazin-1-yl)phenyl]-5-[2-(triisopropylsilyl)ethynyl]pyrido[2,3-d]pyrimidine-2,7-diamine

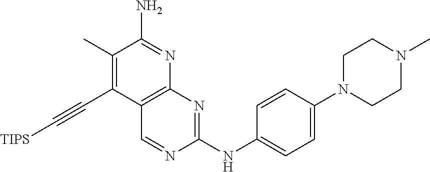

General Procedure 3 was applied to 2-methanesulfonyl-6-methyl-5-[2-(triisopropylsilyl)ethynyl]pyrido[2,3-d]pyrimidin-7-amine (1.00 g, 2.39 mmol), 4-(4-methylpiperazin-1-yl)aniline (590 mg, 3.10 mmol) and trifluoroacetic acid (0.350 mL, 4.78 mmol) in dioxane (10 mL), stirred overnight at 100° C. The residue was purified by reverse phase flash column chromatography eluting with acetonitrile (30-70%) in water (0.1% formic acid), to yield the title compound as a red solid (230 mg, 18%).

### 3-Cyclopentyl-1-(6-methyl-2-{[4-(4-methylpiperazin-1-yl)phenyl]amino}-5-[2-(triisopropylsilyl) ethynyl]pyrido[2,3-d]pyrimidin-7-yl)urea

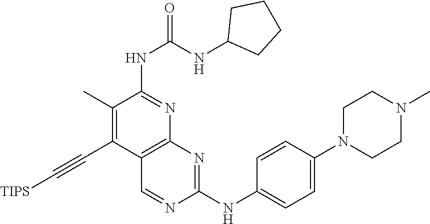

General Procedure 7 was applied to 6-methyl-N2-[4-(4-methylpiperazin-1-yl)phenyl]-5-[2-(triisopropylsilyl)ethynyl]pyrido[2,3-d]pyrimidine-2,7-diamine (230 mg, 0.434 mmol), cyclopentanamine (40.6 mg, 0.477 mmol), CDI (140 mg, 0.868 mmol) and diisopropylethylamine (0.120 mL, 0.868 mmol) in dichloromethane (8.0 mL). The residue was purified by flash column chromatography eluting with ethyl acetate (30%) in 40-60 petroleum ether to afford the title compound as a yellow solid (142 mg, 51%).

### 3-Cyclopentyl-1-(5-ethynyl-6-methyl-2-{[4-(4-methylpiperazin-1-yl)phenyl]amino}pyrido[2,3-d]pyrimidin-7-yl)urea

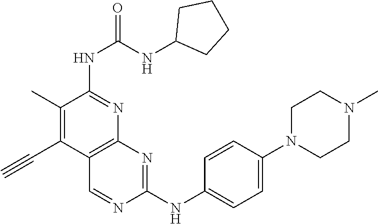 [SMILES: C#Cc1c(C)c(NC(=O)NC2CCCC2)nc2nc(Nc3ccc(N4CCN(C)CC4)cc3)ncc12]

General Procedure 1 was applied to 3-cyclopentyl-1-(6-methyl-2-{[4-(4-methylpiperazin-1-yl)phenyl]amino}-5-[2-(triisopropylsilyl)ethynyl]pyrido[2,3-d]pyrimidin-7-yl)urea (150 mg, 0.234 mmol) and potassium fluoride (136 mg, 2.34 mmol) in THF (2.0 mL), DMF (2.0 mL) and water (0.5 mL), stirred for 1 hour at 60° C. The residue was purified by reverse phase flash column chromatography eluting with acetonitrile (10-80%) in water (0.1% formic acid), to yield the title compound as a yellow solid (36.0 mg, 31%).

(ES, m/z) [M+H]⁺=485.10

¹H-NMR (300 MHz, DMSO-d₆) δ 10.66 (d, J=7.2 Hz, 1H), 9.89 (s, 1H), 9.05 (s, 1H), 8.81 (s, 1H), 7.85 (d, J=8.5 Hz, 2H), 6.94-6.77 (m, 2H), 5.25 (s, 1H), 4.21 (s, 1H), 3.08 (dd, J=6.4, 3.6 Hz, 4H), 2.48 (d, J=4.9 Hz, 3H), 2.45 (d, J=2.2 Hz, 4H), 2.23 (s, 3H), 1.89 (td, J=17.5, 15.0, 6.7 Hz, 4H), 1.69 (dd, J=34.7, 8.1 Hz, 4H).

### Example 73

### 2-Methanesulfinyl-N-methyl-5-[2-(triisopropylsilyl)ethynyl]pyrido[2,3-d]pyrimidin-7-amine

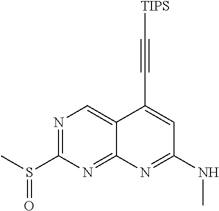

General Procedure 2 was applied to N-methyl-2-(methylsulfanyl)-5-[2-(triisopropylsilyl)ethynyl]pyrido[2,3-d]pyrimidin-7-amine (2.00 g, 5.17 mmol) and m-CPBA (0.89 g, 5.17 mmol) in dichloromethane (20 mL). The residue was purified by flash column chromatography eluting with ethyl acetate (25%) in 40-60 petroleum ether to afford the title compound as a yellow solid (1.98 g, 95%).

### N⁷-Methyl-N²-[4-(4-methylpiperazin-1-yl)phenyl]-5-[2-(triisopropylsilyl)ethynyl]pyrido[2,3-d]pyrimidine-2.7-diamine

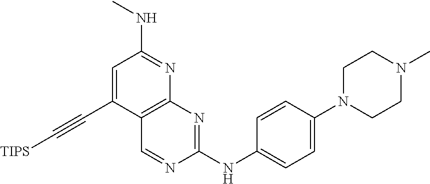

General Procedure 3 was applied to 2-methanesulfinyl-N-methyl-5-[2-(triisopropylsilyl)ethynyl]pyrido[2,3-d]pyrimidin-7-amine (1.89 g, 4.69 mmol), 4-(4-methylpiperazin-1-yl)aniline (1.17 g, 6.10 mmol) and trifluoroacetic acid (0.700 mL, 9.39 mmol) in 2-methyl-2-butanol (20 mL), stirred for 1 hour at 100° C. The residue was purified by reverse phase flash column chromatography eluting with acetonitrile (10-60%) in water (0.1% formic acid), to yield the title compound as a reddish brown solid (426 mg, 17%).

### 3-Cyclopentyl-1-methyl-1-(2-{[4-(4-methylpiperazin-1-yl)phenyl]amino}-5-[2-(triisopropylsilyl) ethynyl]pyrido[2,3-d]pyrimidin-7-yl)urea

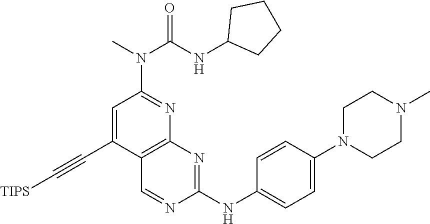

A solution of N⁷-methyl-N²-[4-(4-methylpiperazin-1-yl)phenyl]-5-[2-(triisopropylsilyl)ethynyl]pyrido[2,3-d]pyrimidine-2,7-diamine (200 mg, 0.377 mmol, 1.0 eq.), isocyanatocyclopentane (46.15 mg, 0.415 mmol, 1.1 eq.) and sodium hydride (18.1 mg, 0.754 mmol, 2.0 eq.) in THF (2.0 mL) was stirred for 4 hours at room temperature. The reaction was quenched with saturated aqueous ammonium chloride at 0° C. The aqueous layer was extracted with ethyl acetate (2×10 mL). The combined organic layers were concentrated under reduced pressure. The residue was purified by reverse phase flash column chromatography eluting with acetonitrile (30-100%) in water (0.1% formic acid), to yield the title compound as a reddish brown yellow solid (138 mg, 57%).

### 3-Cyclopentyl-1-(5-ethynyl-2-{[4-(4-methylpiperazin-1-yl)phenyl]amino}pyrido[2,3-d]pyrimidin-7-yl)-1-methylurea

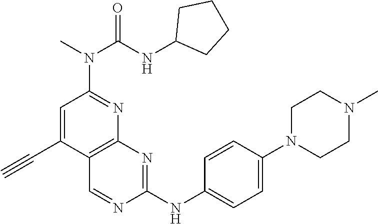 [SMILES: C#Cc1cc(N(C)C(=O)NC2CCCC2)nc2nc(Nc3ccc(N4CCN(C)CC4)cc3)ncc12]

General Procedure 1 was applied to 3-cyclopentyl-1-methyl-1-(2-{[4-(4-methylpiperazin-1-yl)phenyl]amino}-5-[2-(triisopropylsilyl)ethynyl]pyrido[2,3-d]pyrimidin-7-yl)urea (100 mg, 0.156 mmol) and potassium fluoride (90.6 mg, 1.56 mmol) in THF (2.0 mL), DMF (2.0 mL) and water (0.5 mL), stirred for 1 hour at 60° C. The residue was purified by reverse phase flash column chromatography eluting with acetonitrile (10-70%) in water (0.1% formic acid), to yield the title compound as a yellow solid (21.0 mg, 28%).

(ES, m/z): [M+H]⁺=485.10

¹H-NMR (300 MHz, DMSO-d₆) δ 10.76 (s, 1H), 10.07 (s, 1H), 9.14 (s, 1H), 7.88 (d, J=8.4 Hz, 2H), 7.31 (s, 1H), 7.04-6.84 (m, 2H), 5.10 (s, 1H), 4.15 (d, J=5.8 Hz, 1H), 3.10 (s, 3H), 2.67 (s, 3H), 2.00-1.74 (m, 4H), 1.73-1.45 (m, 4H).

### Example 74

### 3-Cyclopentyl-3-methyl-1-(2-{[4-(4-methylpiperazin-1-yl)phenyl]amino}-5-[2-(triisopropylsilyl)ethynyl]pyrido[2,3-d]pyrimidin-7-yl)urea

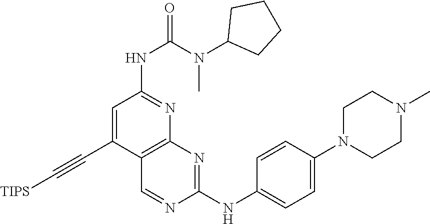

A solution of N²-[4-(4-methylpiperazin-1-yl)phenyl]-5-[2-(triisopropylsilyl)ethynyl]pyrido[2,3-d]pyrimidine-2,7-diamine (300 mg, 0.582 mmol, 1.0 eq.), N-methylcyclopentanamine (69.2 mg, 0.698 mmol, 1.2 eq.), CDI (188 mg, 1.16 mmol, 2.0 eq.) and diisopropylethylamine (0.160 mL, 1.16 mmol, 2.0 eq.) in dichloromethane (5.0 mL) was stirred for 3 hours at room temperature. The resulting mixture was concentrated under reduced pressure. The residue was purified by flash column chromatography eluting with methanol (10%) in dichloromethane to afford the title compound as a red solid (320 mg, 86%).

### 3-Cyclopentyl-1-(5-ethynyl-2-{[4-(4-methylpiperazin-1-yl)phenyl]amino}pyrido[2,3-d]pyrimidin-7-yl)-3-methylurea

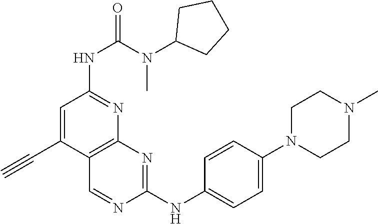 [SMILES: C#Cc1cc(NC(=O)N(C)C2CCCC2)nc2nc(Nc3ccc(N4CCN(C)CC4)cc3)ncc12]

General Procedure 1 was applied to 3-cyclopentyl-3-methyl-1-(2-{[4-(4-methylpiperazin-1-yl)phenyl]amino}-5-[2-(triisopropylsilyl)ethynyl]pyrido[2,3-d]pyrimidin-7-yl)urea (150 mg, 0.234 mmol) and potassium fluoride (136 mg, 2.34 mmol) in THF (2.0 mL), DMF (2.0 mL) and water (0.5 mL), stirred for 1 hour at 60° C. The residue was purified by reverse phase flash column chromatography eluting with acetonitrile (10-50%) in water (0.1% formic acid), to yield the title compound as a reddish brown solid (33.0 mg, 29%).

(ES, m/z) [M+H]⁺=485.15

¹H-NMR (300 MHz, DMSO-d₆) δ 9.91 (d, J=56.3 Hz, 2H), 9.11 (s, 1H), 7.83 (d, J=9.1 Hz, 2H), 7.20 (d, J=50.9 Hz, 1H), 7.07-6.91 (m, 2H), 5.02 (s, 1H), 4.61 (s, 1H), 3.85-3.11 (m, 8H), 2.97-2.80 (m, 6H), 1.87-1.43 (m, 8H).

### Example 75

### 3-Cyclopentyl-1-(2-([4-(morpholin-4-yl)phenyl]amino-5-[2-(triisopropylsilyl)ethynyl]pyrido[2,3-d]pyrimidin-7-yl)urea

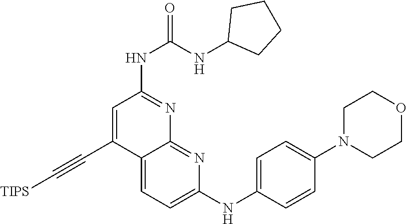

General Procedure 3 was applied to 3-cyclopentyl-1-(2-methanesulfinyl-5-[2-(triisopropylsilyl)ethynyl]pyrido[2,3-d]pyrimidin-7-ylurea (280 mg, 0.560 mmol) and 4-(morpholin-4-yl)aniline (149 mg, 0.840 mmol) and trifluoroacetic acid (128 mg, 1.12 mmol) in 2-methyl-2-butanol (3 mL), stirred for 2 hours at 100° C. The residue was purified by flash column chromatography, eluting with ethyl acetate (20%) in 40-60 petroleum ether to afford the title compound as a yellow solid (300 mg, 87%).

### 3-Cyclopentyl-1-(5-ethynyl-2-([4-(morpholin-4-yl)phenyl]aminopyrido[2,3-d]pyrimidin-7-yl)urea

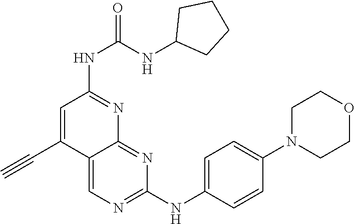 [SMILES: C#Cc1cc(NC(=O)NC2CCCC2)nc2nc(Nc3ccc(N4CCOCC4)cc3)ncc12]

General Procedure 1 was applied to 3-cyclopentyl-1-(2-([4-(morpholin-4-yl)phenyl]amino-5-[2-(triisopropylsilyl)ethynyl]pyrido[2,3-d]pyrimidin-7-yl)urea (300 mg, 0.489 mmol) and potassium fluoride (284 mg, 4.89 mmol) in THF (3.0 mL), DMF (3.0 mL) and water (300 uL), stirred for 2 hours at 60° C. The residue was purified by reverse phase flash column chromatography eluting with acetonitrile (10-50%) in water (0.1% trifluoroacetic acid) to yield the title compound as an orange solid (5.00 mg, 2%).

(ES, m/z): [M+H]⁺=458.20

¹H-NMR (400 MHz, DMSO-d₆) δ 10.01 (d, J=17.5 Hz, 2H), 9.05 (s, 1H), 7.86 (s, 2H), 7.16 (s, 1H), 6.90 (d, J=9.0 Hz, 2H), 5.05 (s, 1H), 4.14 (s, 1H), 3.75 (t, J=4.8 Hz, 4H), 3.05 (t, J=4.8 Hz, 4H), 1.95-1.83 (m, 2H), 1.80 (s, 2H), 1.70 (s, 2H), 1.56 (s, 2H).

### Example 76

### 1-(4-Nitrophenyl)-4-(oxetan-3-yl)piperazine

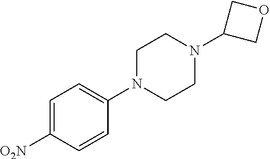 [SMILES: O=[N+]([O-])c1ccc(N2CCN(C3COC3)CC2)cc1]

A solution of 4-fluoronitrobenzene (5.00 g, 35.4 mmol, 1.0 eq.) and 1-(oxetan-3-yl)piperazine (5.29 g, 37.2 mmol, 1.05 eq.) in acetnitrile (50 mL) was stirred overnight at 80° C. The mixture was allowed to cool down to room temperature. The precipitated solids were collected by filtration, washing with acetonitrile (20 mL) to yield the title compound as a yellow solid (8.75 g, 94%).

### 4-[4-(Oxetan-3-yl)piperazin-1-yl]aniline

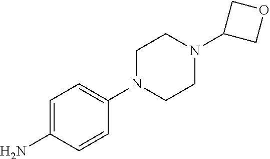 [SMILES: Nc1ccc(N2CCN(C3COC3)CC2)cc1]

General Procedure 5 was applied to 1-(4-nitrophenyl)-4-(oxetan-3-yl)piperazine (2.00 g, 7.59 mmol) and palladium on carbon (10%, 404 mg, 0.380 mmol) in ethanol (20 mL). The mixture was hydrogenated at 50° C. to yield the title compound as a black solid (1.52 g, 86%).

### 3-Cyclopentyl-1-[2-({4-[4-(oxetan-3-yl)piperazin-1-yl]phenyl}amino)-5-[2-(triisopropylsilyl)ethynyl]pyrido[2,3-d]pyrimidin-7-yl]urea

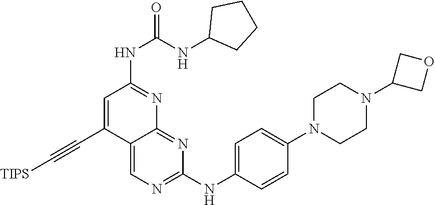

General Procedure 3 was applied to 3-cyclopentyl-1-{2-methanesulfonyl-5-[2-(triisopropylsilyl)ethynyl]pyrido[2,3-d]pyrimidin-7-yl}urea (200 mg, 0.388 mmol), 4-[4-(oxetan-3-yl)piperazin-1-yl]aniline (117 mg, 0.504 mmol) and trifluoroacetic acid (60.0 μL, 0.776 mmol) in 2-methyl-2-butanol (2.0 mL), stirred for 1 hour at 100° C. to yield the title compound as a yellow solid (156 mg, 60%).

### 3-Cyclopentyl-1-[5-ethynyl-2-({4-[4-(oxetan-3-yl)piperazin-1-yl]phenyl}amino)pyrido[2,3-d]pyrimidin-7-yl]urea

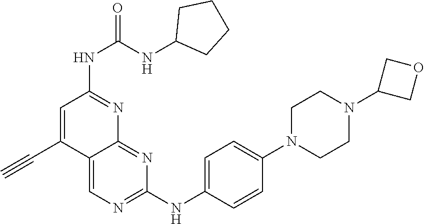 [SMILES: C#Cc1cc(NC(=O)NC2CCCC2)nc2nc(Nc3ccc(N4CCN(C5COC5)CC4)cc3)ncc12]

General Procedure 1 was applied to 3-cyclopentyl-1-[2-({4-[4-(oxetan-3-yl)piperazin-1-yl]phenyl}amino)-5-[2-(triisopropylsilyl)ethynyl]pyrido[2,3-d]pyrimidin-7-yl]urea (100 mg, 0.149 mmol) and potassium fluoride (86.8 mg, 1.49 mmol) in THF (2.0 mL), DMF (2.0 mL) and water (0.50 mL) was stirred for 1 hour at 60° C. The residue was purified by reverse phase flash column chromatography eluting with acetonitrile (10-60%) in water (0.1% formic acid), to yield the title compound as a yellow solid (12.0 mg, 15%).

(ES, m/z) [M+H]⁺=513.25

¹H-NMR (300 MHz, DMSO-d₆) δ 10.00 (d, J=15.4 Hz, 2H), 9.50 (s, 1H), 9.05 (s, 1H), 7.84 (s, 2H), 7.15 (s, 1H), 6.90 (d, J=9.1 Hz, 2H), 5.05 (s, 1H), 4.58 (t, J=6.5 Hz, 2H), 4.48 (t, J=6.0 Hz, 2H), 3.46 (q, J=6.3 Hz, 1H), 3.12 (t, J=4.9 Hz, 4H), 2.42 (t, J=5.0 Hz, 4H), 1.95-1.66 (m, 6H), 1.61-1.48 (m, 2H).

### Example 77

### N-[4-((7-[(Cyclopentylcarbamoyl)amino]-5-[2-(triisopropylsilyl)ethynyl]pyrido[2,3-d]pyrimidin-2-ylamino)phenyl]-N-methylacetamide

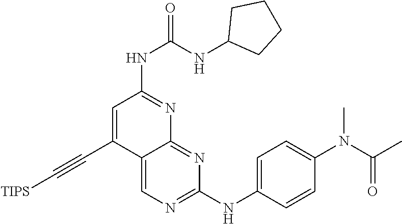

General Procedure 3 was applied to 3-cyclopentyl-1-(2-methanesulfonyl-5-[2-(triisopropylsilyl)ethynyl]pyrido[2,3-d]pyrimidin-7-ylurea (300 mg, 0.582 mmol), N-(4-aminophenyl)-N-methylacetamide (143 mg, 0.873 mmol) and trifluoroacetic acid (86.4 uL, 1.16 mmol) in 2-methyl-2-butanol (3.0 mL), stirred for 2 hours at 100° C. The residue was purified by flash column chromatography eluting with ethyl acetate (20%) in 40-60 petroleum ether to afford the title compound as a yellow solid (300 mg, 86%).

### N-[4-((7-[(Cyclopentylcarbamoyl)amino]-5-ethynyl)pyrido[2,3-d]pyrimidin-2-ylamino)phenyl]-N-methylacetamide

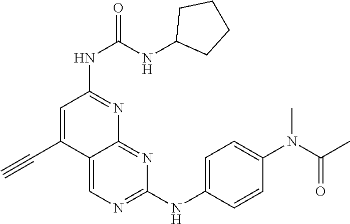 [SMILES: C#Cc1cc(NC(=O)NC2CCCC2)nc2nc(Nc3ccc(N(C)C(C)=O)cc3)ncc12]

General Procedure 1 was applied to N-[4-((7-[(cyclopentylcarbamoyl)amino]-5-[2-(triisopropylsilyl)ethynyl]pyrido[2,3-d]pyrimidin-2-ylamino)phenyl]-N-methylacetamide (300 mg, 0.500 mmol) and potassium fluoride (290 mg, 5.00 mmol) in THF (3.0 mL), DMF (3.0 mL) and water (300 uL), stirred for 2 hours at 60° C. The residue was purified by reverse phase flash column chromatography eluting with acetonitrile (10-50%) in water (0.1% trifluoroacetic acid) to yield the title compound as a yellow solid (30.0 mg, 13%).

(ES, m/z): [M+H]⁺=444.20

¹H-NMR (400 MHz, DMSO-d₆) δ 10.32 (s, 1H), 10.09 (s, 1H), 9.38 (s, 1H), 9.13 (s, 1H), 8.05 (d, J=8.4 Hz, 2H), 7.26 (d, J=8.2 Hz, 3H), 5.08 (s, 1H), 4.14 (d, J=6.1 Hz, 1H), 3.14 (s, 3H), 1.88 (dt, J=13.1, 6.6 Hz, 2H), 1.78 (s, 5H), 1.70 (d, J=7.2 Hz, 2H), 1.60-1.49 (m, 2H).

### Example 78

### 2-Chloro-N-methyl-N-(4-nitrophenyl)acetamide

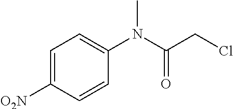

A solution of 4-nitro-N-methylaniline (10.0 g, 65.7 mmol, 1.0 eq.) and chloroacetic anhydride (12.3 g, 72.3 mmol, 1.1 eq.) in ethyl acetate (20 mL) was stirred for 1 hour at room temperature. The resulting mixture was extracted with ethyl acetate (3×200 mL). The combined organic layers were washed with saturated aqueous sodium carbonate (2×200 mL), dried (Na₂SO₄) and concentrated under reduced pressure to yield the title compound as a yellow solid (15.0 g, 99%). 2-(Azetidin-1-yl)-N-methyl-N-(4-nitrophenyl)acetamide

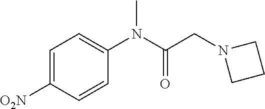 [SMILES: CN(C(=O)CN1CCC1)c1ccc([N+](=O)[O-])cc1]

A solution of 2-chloro-N-methyl-N-(4-nitrophenyl)acetamide (10.0 g, 43.7 mmol, 1.0 eq.) in acetonitrile (100 mL) was treated with azetidine hydrochloride (6.14 g, 65.6 mmol, 1.5 eq.) followed by potassium carbonate (18.1 g, 131 mmol, 3.0 eq.). The resulting mixture was stirred for 1 hour at 50° C. The residue was purified by flash column chromatography eluting with methanol (1.3%) in dichloromethane to afford the title compound as a light yellow solid (2.00 g, 18%).

### N-(4-Aminophenyl)-2-(azetidin-1-vi)-N-methylacetamide

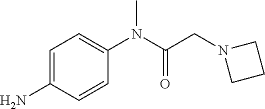 [SMILES: CN(C(=O)CN1CCC1)c1ccc(N)cc1]

A solution of 2-(azetidin-1-yl)-N-methyl-N-(4-nitrophenyl)acetamide (500 mg, 2.00 mmol, 1.0 eq.) in dioxane (10 mL) and water (4.0 mL) was treated with ammonium chloride (1.07 g, 20.0 mmol, 10 eq.). After 5 minutes stirring at 60° C. iron (1.12 g, 20.0 mmol, 10 eq.) was added in portions at 60° C. The resulting mixture was stirred for 1 hour at 60° C. then filtered, washing with dichloromethane (3×50 mL). The filtrate was concentrated under reduced pressure. The resulting mixture was extracted with dichloromethane (3×50 mL). The combined organic layers were washed with saturated aqueous sodium carbonate (2×50 mL), dried (Na₂SO₄) and concentrated under reduced pressure to yield the title compound as a light yellow solid (300 mg, 68%).

### 2-(Azetidin-1-yl)-N-[4-({7-[(cyclopentylcarbamoyl)amino]-5-[2-(triisoprooylsilyl)ethynyl]pyrido[2,3-d]pyrimidin-2-yl}amino)Phenyl]-N-methylacetamide

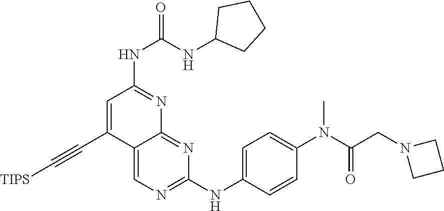

General Procedure 3 was applied to 3-cyclopentyl-1-{2-methanesulfonyl-5-[2-(triisopropylsilyl)ethynyl]pyrido[2,3-d]pyrimidin-7-yl}urea (300 mg, 0.582 mmol), N-(4-aminophenyl)-2-(azetidin-1-yl)-N-methylacetamide (191 mg, 0.873 mmol) and trifluoroacetic acid (132 mg, 1.16 mmol) in 2-methyl-2-butanol (2.0 mL). The resulting mixture was stirred overnight at room temperature. The residue was purified by reverse phase flash column chromatography eluting acetonitrile (50-100%) in water (0.1% formic acid) to yield the title compound as a light yellow solid (200 mg, 52%).

### 2-(Azetidin-1-yl)-N-[4-((7-[(cyclopentylcarbamoyl)amino]-5-ethynyl)pyrido[2,3-d]pyrimidin-2-ylamino)phenyl]-N-methylacetamide

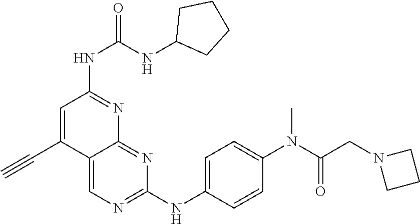 [SMILES: C#Cc1cc(NC(=O)NC2CCCC2)nc2nc(Nc3ccc(N(C)C(=O)CN4CCC4)cc3)ncc12]

A solution of 2-(azetidin-1-yl)-N-[4-((7-[(cyclopentylcarbamoyl)amino]-5-[2-(triisopropylsilyl)ethynyl]pyrido[2,3-d]pyrimidin-2-ylamino)phenyl]-N-methylacetamide (80.0 mg, 0.122 mmol, 1.0 eq.) and TBAF (20.1 mg, 77.0 μmol, 0.63 eq.) in THF (1 mL) was stirred for 5 minutes at room temperature. The resulting mixture was extracted with chloroform: i-propanol (3:1) (2×10 mL). The combined organic layers were washed with water (3×10 mL), dried (Na₂SO₄) and concentrated under reduced pressure. The residue was triturated with ethyl acetate to yield the title compound as a yellow solid (17.0 mg, 28%).

(ES, m/z): [M+H]⁺=499.20

¹H-NMR (400 MHz, DMSO-d₆) δ 10.36 (s, 1H), 10.15 (s, 1H), 9.39 (s, 1H), 9.14 (s, 1H), 8.07 (d, J=8.4 Hz, 2H), 7.43-7.10 (m, 4H), 5.12 (s, 1H), 4.11 (dd, J=11.7, 6.1 Hz, 1H), 2.00 (d, J=7.7 Hz, 2H), 1.93-1.85 (m, 2H), 1.84-1.76 (m, 3H), 1.70 (d, J=7.6 Hz, 3H), 1.23 (s, 5H).

### Example 79

### 3-Cyclopentyl-1-[2-((4-[2-(dimethylamino)ethoxyl]phenylamino)-5-[2-(triisopropylsilyl)ethynyl]pyrido[2,3-d]pyrimidin-7-yl]urea

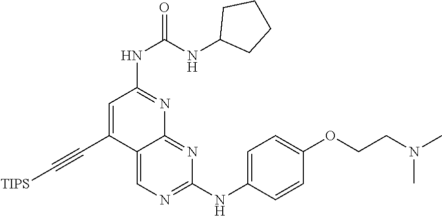

General Procedure 3 was applied to 3-cyclopentyl-1-(2-methanesulfinyl-5-[2-(triisopropylsilyl)ethynyl]pyrido[2,3-d]pyrimidin-7-ylurea (300 mg, 0.600 mmol), 4-[2-(dimethylamino)ethoxy]aniline (162 mg, 0.900 mmol) and trifluoroacetic acid (137 mg, 1.20 mmol) in 2-methyl-2-butanol (3.0 mL), stirred for 2 hours at 100° C. The residue was purified by flash column chromatography eluting with ethyl acetate (20%) in 40-60 petroleum ether to afford the title compound as a yellow solid (230 mg, 62%).

### 3-Cyclopentyl-1-[2-((4-[2-(dimethylamino)ethoxyl]phenylamino)-5-ethynyl)pyrido[2,3-d]pyrimidin-7-yl]urea

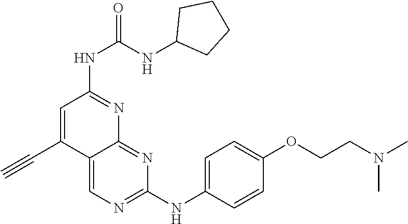 [SMILES: C#Cc1cc(NC(=O)NC2CCCC2)nc2nc(Nc3ccc(OCCN(C)C)cc3)ncc12]

General Procedure 1 was applied to 3-cyclopentyl-1-[2-((4-[2-(dimethylamino)ethoxy]phenylamino)-5-[2-(triisopropylsilyl)ethynyl]pyrido[2,3-d]pyrimidin-7-yl]urea (230 mg, 0.373 mmol) and potassium fluoride (217 mg, 3.73 mmol) in THF (3.0 mL), DMF (3.0 mL) and water (230 μL), stirred for 2 hours at 60° C. The residue was purified by reverse phase flash column chromatography eluting with acetonitrile (10-50%) in water

### (0.1% trifluoroacetic acid) to yield the title compound as a yellow solid (33.0 mg, 19%)

(ES, m/z): [M+H]⁺=460.15

¹H-NMR (400 MHz, DMSO-d₆) δ 10.04 (s, 2H), 9.49 (s, 1H), 9.07 (s, 1H), 7.90 (s, 2H), 7.16 (s, 1H), 6.88 (d, J=9.0 Hz, 2H), 5.05 (s, 1H), 4.14 (s, 1H), 4.03 (t, J=5.8 Hz, 2H), 2.64 (t, J=5.8 Hz, 2H), 2.23 (s, 6H), 1.87 (dd, J=12.9, 6.1 Hz, 2H), 1.80 (s, 2H), 1.69 (d, J=9.1 Hz, 2H), 1.55 (s, 2H).

### Example 80

### tert-Butyl 3-(4-nitrophenoxy)azetidine-1-carboxylate

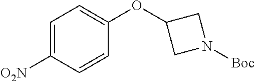 [SMILES: CC(C)(C)OC(=O)N1CC(Oc2ccc([N+](=O)[O-])cc2)C1]

A solution of 4-nitrophenol (5.00 g, 35.9 mmol, 1.0 eq.), potassium carbonate (9.93 g, 71.9 mmol, 2.0 eq.), tert-butyl 3-bromoazetidine-1-carboxylate (8.49 g, 35.9 mmol, 1.0 eq.) and DMF (50 mL) was stirred overnight at 110° C. The resulting mixture was extracted with ethyl acetate (3×150 mL). The combined organic layers were washed with bring (2×300 mL), dried (Na₂SO₄) and concentrated under reduced pressure. The residue was purified by flash column chromatography eluting with ethyl acetate (12%) in 40-60 petroleum ether to afford the title compound as a yellow solid (8.58 g, 81%).

### 3-(4-Nitrophenoxy)azetidine

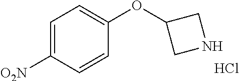 [SMILES: O=[N+]([O-])c1ccc(OC2CNC2)cc1 ; Cl]

A solution of tert-butyl 3-(4-nitrophenoxy)azetidine-1-carboxylate (5.50 g, 18.7 mmol, 1.0 eq.) and 4M hydrochloric acid in methanol (55 mL, 220 mmol, 12 eq.) at room temperature for 2 hours. The resulting mixture was concentrated under reduced pressure, to afford the title compound as a brown yellow solid (4.52 g, 124%).

### 1-Methyl-3-(4-nitrophenoxy)azetidine

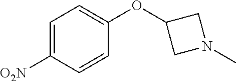

To a stirred solution of 3-(4-nitrophenoxy)azetidine (4.52 g, 23.3 mmol, 1.0 eq.), formaldehyde (699 mg, 23.3 mmol, 1.0 eq.) and methanol (45.2 mL) was added sodium cyanoborohydride (4.39 g, 69.8 mmol, 3.0 eq.) at 0° C. under nitrogen atmosphere. The resulting mixture was stirred overnight at room temperature then extracted with ethyl acetate (3×100 mL). The combined organic layers were washed with brine (200 mL), dried (Na₂SO₄) and concentrated under reduced pressure. The residue was purified by flash column chromatography eluting with ethyl acetate (20%) in 40-60 petroleum ether to afford the title compound as a yellow solid (3.02 g, 62%).

### 4-[(1-Methylazetidin-3-yl)oxyl]aniline

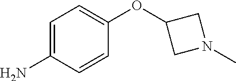

General Procedure 5 was applied to 1-methyl-3-(4-nitrophenoxy)azetidine (800 mg, 3.84 mmol) and palladium on carbon (204 mg, 1.92 mmol) in methanol (60 mL) to yield the title compound as a grey white solid (486 mg, 28%).

### 3-Cyclopentyl-1-[2-((4-[(1-methylazetidin-3-yl)oxy]phenylamino)-5-[2-(triisopropylsilyl)ethynyl]pyrido[2,3-d]pyrimidin-7-yl]urea

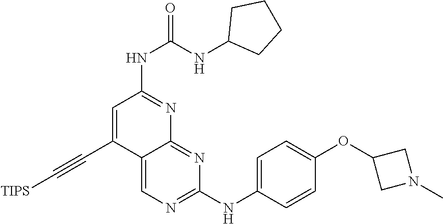

General Procedure 3 was applied to 3-cyclopentyl-1-(2-methanesulfinyl-5-[2-(triisopropylsilyl)ethynyl]pyrido[2,3-d]pyrimidin-7-ylurea (400 mg, 0.800 mmol), 4-[(1-methylazetidin-3-yl)oxy]aniline (214 mg, 1.20 mmol) and trifluoroacetic acid (119 μL, 1.60 mmol) and 2-butanol (4 mL), stirred overnight at 50° C. The residue was purified by reverse phase flash column chromatography eluting with acetonitrile (10-90%) in water (0.1% formic acid), to yield the title compound as a brown yellow solid (191 mg, 39%).

### 3-Cyclopentyl-1-[5-ethynyl-2-((4-[(1-methylazetidin-3-yl)oxy]phenylamino)pyrido[2,3-d]pyrimidin-7-yl]urea

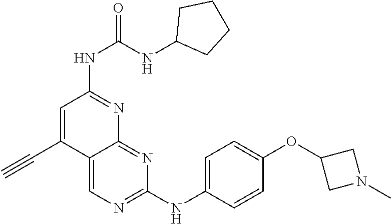 [SMILES: C#Cc1cc(NC(=O)NC2CCCC2)nc2nc(Nc3ccc(OC4CN(C)C4)cc3)ncc12]

A solution of 3-cyclopentyl-1-[2-((4-[(1-methylazetidin-3-yl)oxy]phenylamino)-5-[2-(triisopropylsilyl)ethynyl]pyrido[2,3-d]pyrimidin-7-yl]urea (100 mg, 0.163 mmol, 1.0 eq.) and TBAF (100 uL, 0.05 eq.) in tetrahydrofuran (1.0 mL) was stirred overnight at room temperature. The residue was purified by trituration with water (2.0 mL). The precipitated solids were collected by filtration, washing with THF (3×1 mL) and water (3×2 mL). The resulting solid was dried under infrared light to yield the title compound as a yellow solid (3.50 mg, 5%).

(ES, m/z): [M+H]⁺=458.25

¹H NMR (400 MHz, DMSO-d₆) δ 10.04 (s, 2H), 9.53 (s, 1H), 9.07 (s, 1H), 7.90 (s, 2H), 7.16 (s, 1H), 6.82-6.70 (m, 2H), 5.05 (s, 1H), 4.71 (p, J=5.7 Hz, 1H), 4.15 (s, 1H), 3.73 (td, J=6.1, 1.9 Hz, 2H), 2.96 (td, J=5.7, 1.9 Hz, 2H), 2.29 (s, 3H), 1.96-1.85 (m, 2H), 1.79 (q, J=9.3, 7.6 Hz, 2H), 1.75-1.66 (m, 2H), 1.61-1.50 (m, 2H), 1.24 (d, J=12.9 Hz, 1H), 1.09-0.96 (m, 1H).

### Example 81

### 3-Cyclopentyl-1-{2-[(4-methanesulfonylphenyl)amino]-5-[2-(triisopropylsilyl)ethynyl]pyrido[2,3-d]pyrimidin-7-yl}urea

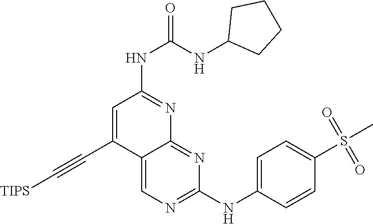

General Procedure 3 was applied to 3-cyclopentyl-1-{2-methanesulfonyl-5-[2-(triisopropylsilyl)ethynyl]pyrido[2,3-d]pyrimidin-7-yl}urea (330 mg, 0.640 mmol), 4-methylsulfonylaniline (164 mg, 0.960 mmol) and trifluoroacetic acid (146 mg, 1.28 mmol) in butan-2-ol (6.6 mL), stirred for 4 hours at 100° C. The residue was purified by trituration with ethyl acetate (50 mL) to yield the title compound as a yellow solid (160 mg, 41%).

### 3-Cyclopentyl-1-{5-ethynyl-2-[(4-methanesulfonylphenyl)amino]pyrido[2,3-d]pyrimidin-7-yl}urea

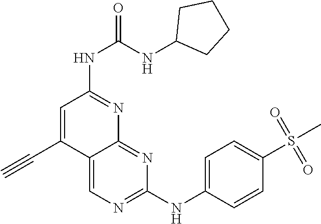 [SMILES: C#Cc1cc(NC(=O)NC2CCCC2)nc2nc(Nc3ccc(S(C)(=O)=O)cc3)ncc12]

General Procedure 1 was applied to 3-cyclopentyl-1-{2-[(4-methanesulfonylphenyl)amino]-5-[2-(triisopropylsilyl) ethynyl]pyrido[2,3-d]pyrimidin-7-yl}urea (160 mg, 0.264 mmol) and potassium fluoride (153 mg, 2.63 mmol) in DMF (1.6 mL) and THF (1.6 mL) and water (0.30 mL), stirred for 4 hours at 60° C. The residue was purified by trituration with acetonitrile and water (1:1, 20 mL) to yield the title compound as a yellow solid (42.0 mg, 35%).

(ES, m/z): [M+H]⁺=451

¹H-NMR (300 MHz, DMSO-d₆) δ 10.69 (s, 1H), 10.16 (s, 1H), 9.42-9.30 (m, 1H), 9.20 (s, 1H), 8.26 (d, J=8.8 Hz, 2H), 7.83 (d, J=8.8 Hz, 2H), 7.32 (s, 1H), 5.11 (s, 1H), 4.23-4.05 (m, 1H), 3.18 (s, 3H), 1.99-1.66 (m, 6H), 1.63-1.52 (m, 2H).

### Example 82

### 3-Cyclopentyl-1-(2-([4-(dimethylsulfamoyl)phenyl]amino-5-[2-(triisopropylsilyl)ethynyl]pyrido[2,3-d]pyrimidin-7-yl)urea

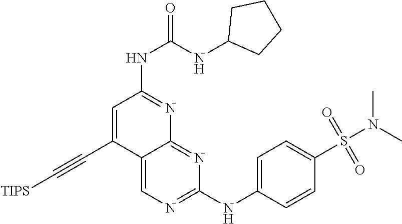

General Procedure 3 was applied to 3-cyclopentyl-1-(2-methanesulfinyl-5-[2-(triisopropylsilyl)ethynyl]pyrido[2,3-d]pyrimidin-7-ylurea (300 mg, 0.600 mmol) and N¹-dimethylsulfanilamide (180 mg, 0.900 mmol) and trifluoroacetic acid (137 mg, 1.20 mmol) in 2-methyl-2-butanol (3.0 mL), stirred for 2 hours at 100° C. The residue was purified by flash column chromatography eluting with ethyl acetate (20%) in 40-60 petroleum ether to afford the title compound as a yellow solid (230 mg, 60%).

### 3-Cyclopentyl-1-(2-([4-(dimethylsulfamoyl)phenyl]amino-5-ethynyl)pyrido[2,3-d]pyrimidin-7-yl)urea

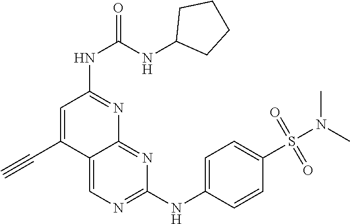 [SMILES: C#Cc1cc(NC(=O)NC2CCCC2)nc2nc(Nc3ccc(S(=O)(=O)N(C)C)cc3)ncc12]

General Procedure 1 was applied to 3-cyclopentyl-1-(2-([4-(dimethylsulfamoyl)phenyl]amino-5-[2-(triisopropylsilyl)ethynyl]pyrido[2,3-d]pyrimidin-7-yl)urea (230 mg, 0.362 mmol) and potassium fluoride (210 mg, 3.62 mmol) in THF (3.0 mL), DMF (3.0 mL) and water (230 uL), stirred for 2 hours at 60° C. The residue was purified by reverse phase flash column chromatography eluting with acetonitrile (10-50%) in water

### (0.1% trifluoroacetic acid) to yield the title compound as a yellow solid (33.0 mg, 19%)

(ES, m/z): [M+H]⁺=479.95

¹H-NMR (400 MHz, DMSO-d₆): δ 10.68 (s, 1H), 10.15 (s, 1H), 9.44 (s, 1H), 9.19 (s, 1H), 8.30-8.23 (m, 2H), 7.69-7.61 (m, 2H), 7.29 (s, 1H), 5.11 (s, 1H), 4.19-4.09 (m, 1H), 2.60 (s, 6H), 1.90 (dt, J=12.9, 6.5 Hz, 2H), 1.84-1.75 (m, 2H), 1.70 (d, J=6.7 Hz, 2H), 1.57 (dd, J=11.4, 6.4 Hz, 2H).

### Example 83

### 2-(Dimethylamino)-N-methyl-N-(4-nitrophenyl)acetamide

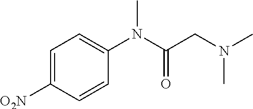 [SMILES: CN(C)CC(=O)N(C)c1ccc([N+](=O)[O-])cc1]

A mixture of 2-chloro-N-methyl-N-(4-nitrophenyl)acetamide (2.50 g, 10.9 mmol, 1 eq.), potassium carbonate (4.55 g, 32.8 mmol, 3.0 eq.) and dimethylamine hydrochloride (1.33 g, 16.4 mmol, 1.5 eq.) in acetonitrile (20 mL) was stirred for 3 hours at 80° C. The mixture was allowed to cool down to room temperature then concentrated under reduced pressure. The residue was purified by flash column chromatography, eluting with ethyl acetate (10%) in dichloromethane to yield the title compound as a reddish brown solid (1.90 g, 73.24%).

¹H-NMR (400 MHz, Chloroform-d): δ 8.29-8.20 (m, 2H), 7.47-7.38 (m, 2H), 3.34 (s, 3H), 2.99 (s, 2H), 2.22 (s, 6H).

### N-(4-Aminophenyl)-2-(dimethylamino)-N-methylacetamide

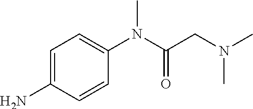

A solution of 2-(dimethylamino)-N-methyl-N-(4-nitrophenyl)acetamide (1.00 g, 4.21 mmol, 1.0 eq.) in 1,4-dioxane (20 mL) and water (4.0 mL) was treated with iron (2.35 g, 42.1 mmol, 10 eq.) and ammonium chloride (2.25 g, 42.1 mmol, 10 eq.) for 4 hours at 60° C. The mixture was allowed to cool down to room temperature then filtered, washing with methanol (3×50 mL). The organic solvent was removed under reduced pressure. The resulting mixture was extracted with ethyl acetate (3×50 mL). The combined organic layers were washed with brine (2×40 mL), dried (Na₂SO₄) and concentrated under reduced pressure. The residue was purified by flash column chromatography, eluting with ethyl acetate (50%) in 40-60 petroleum ether to yield the title compound as a brown yellow solid (540 mg 62%).

¹H-NMR (400 MHz, Chloroform-d) δ 6.99-6.89 (m, 2H), 6.71-6.63 (m, 2H), 3.78 (s, 2H), 3.20 (s, 3H), 2.87 (s, 2H), 2.25 (s, 6H).

### N-[4-{(7-[(Cyclopentylcarbamoyl)amino]-5-[2-(triisopropylsilyl)ethynyl]pyrido[2,3-d]pyrimidin-2-yl}amino)phenyl]-2-(dimethylamino)-N-methylacetamide

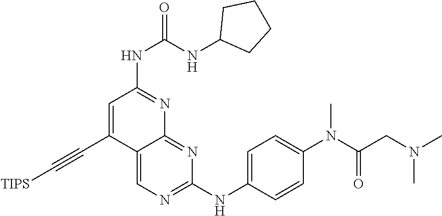

General Procedure 3 was applied to 3-cyclopentyl-1-{2-methanesulfonyl-5-[2-(triisopropylsilyl)ethynyl]pyrido[2,3-d]pyrimidin-7-yl}urea (300 mg, 0.600 mmol), N-(4-aminophenyl)-2-(dimethylamino)-N-methylacetamide (186 mg, 0.900 mmol) and trifluoroacetic acid (0.100 mL, 1.34 mmol) in butan-2-ol (5.0 mL), stirred for 2 hours at 100° C. The residue was purified by flash column chromatography, eluting with methanol (10%) in dichloromethane to yield the title compound as a yellow solid (250 mg, 65%).

### N-[4-({7-[(Cyclopentylcarbamoyl)amino]-5-ethynyl)pyrido[2,3-d]pyrimidin-2-yl}amino)phenyl]-2-(dimethylamino)-N-methylacetamide

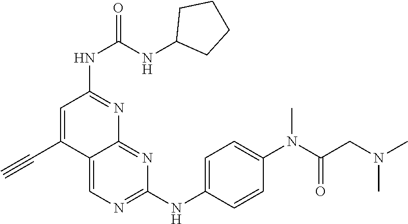 [SMILES: C#Cc1cc(NC(=O)NC2CCCC2)nc2nc(Nc3ccc(N(C)C(=O)CN(C)C)cc3)ncc12]

General Procedure 1 was applied to N-[4-({7-[(cyclopentylcarbamoyl)amino]-5-[2-(triisopropylsilyl)ethynyl]pyrido[2,3-d]pyrimidin-2-yl}amino)phenyl]-2-(dimethylamino)-N-methylacetamide (150 mg, 0.233 mmol) and potassium fluoride (135 mg, 2.33 mmol) in THF (2.0 mL), DMF(1.0 mL), and water (1.0 mL), stirred for 1 hour at 60° C. The crude product was purified by Prep-HPLC eluting with acetonitrile (10-60%) in water (0.1% formic acid), to yield the title compound as a yellow solid (30.0 mg, 26%).

(ES,m/z): [M+H]⁺=487.25

¹H-NMR (400 MHz, DMSO-d₆) δ 10.32 (s, 1H), 10.08 (s, 1H), 9.42 (s, 1H), 9.13 (d, J=1.3 Hz, 1H), 8.05 (d, J=8.4 Hz, 2H), 7.25 (d, J=8.7 Hz, 3H), 5.08 (s, 1H), 4.17-4.10 (m, 1H), 3.14 (s, 3H), 2.84 (s, 2H), 2.13 (s, 6H), 1.89 (dd, J=12.4, 6.4 Hz, 2H), 1.82-1.77 (m, 2H), 1.70 (d, J=7.3 Hz, 2H), 1.56 (dd, J=11.2, 5.9 Hz, 2H).

### Example 84

### N-[2-(Dimethylamino)ethyl]-N-methyl-4-nitrobenzenesulfonamide

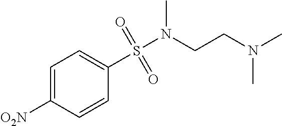 [SMILES: CN(C)CCN(C)S(=O)(=O)c1ccc([N+](=O)[O-])cc1]

To a stirred solution of 4-nitrobenzenesulfonyl chloride (5.00 g, 22.6 mmol, 1.0 eq.) and triethylamine (7.50 mL, 53.9 mmol, 2.4 eq.) in dichloromethane (30 mL) was added [2-(dimethylamino)ethyl](methyl)amine (3.00 mL) in portions at room temperature. The resulting mixture was stirred for 2 hours at room temperature then washed with water (3×100 mL) and concentrated under reduced pressure. The product was precipitated by the addition of MTBE. The precipitated solids were collected by filtration and washed with MTBE (2×5 mL) to yield the title compound as a yellow solid (4.00 g, 61%).

### 4-Amino-N-[2-(dimethylamino)ethyl]-N-methylbenzenesulfonamide

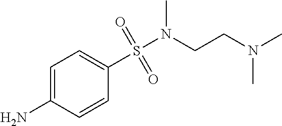 [SMILES: CN(C)CCN(C)S(=O)(=O)c1ccc(N)cc1]

General Procedure 5 was applied to N-[2-(dimethylamino)ethyl]-N-methyl-4-nitrobenzenesulfonamide (400 mg, 1.39 mmol) and palladium on carbon (80.0 mg, 0.752 mmol) in methanol (10 mL) to yield the title compound as a yellow solid (300 mg, 84%).

### 3-Cyclopentyl-1-(2-[(4-([2-(dimethylamino)ethyl](methyl)sulfamoylphenyl)amino]-5-[2-(triisopropylsilyl)ethynyl]pyrido[2,3-d]pyrimidin-7-ylurea

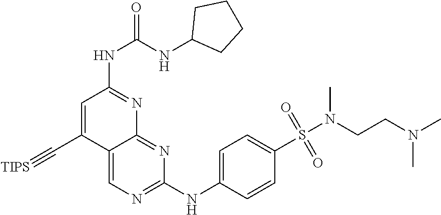

General Procedure 3 was applied to 3-cyclopentyl-1-(2-methanesulfinyl-5-[2-(triisopropylsilyl)ethynyl]pyrido[2,3-d]pyrimidin-7-ylurea (300 mg, 0.600 mmol), 4-amino-N-[2-(dimethylamino)ethyl]-N-methylbenzenesulfonamide (232 mg, 0.900 mmol) and trifluoroacetic acid (137 mg, 1.20 mmol) in butan-2-ol (3.0 mL), stirred overnight at 100° C. The residue was purified by reverse phase flash column chromatography eluting with acetonitrile (30-70%) in water (0.1% formic acid), to yield the title compound as a yellow solid (200 mg, 48%).

### 3-Cyclopentyl-1-(2-[(4-([2-(dimethylamino)ethyl](methyl)sulfamoylphenyl)amino]-5-ethynyl)pyrido[2,3-d]pyrimidin-7-ylurea

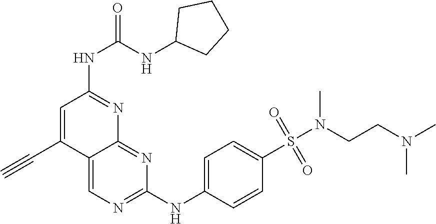 [SMILES: C#Cc1cc(NC(=O)NC2CCCC2)nc2nc(Nc3ccc(S(=O)(=O)N(C)CCN(C)C)cc3)ncc12]

General Procedure 1 was applied to 3-cyclopentyl-1-(2-[(4-([2-(dimethylamino)ethyl](methyl)sulfamoyl)phenyl)amino]-5-[2-(triisopropylsilyl)ethynyl]pyrido[2,3-d]pyrimidin-7-ylurea (150 mg, 0.216 mmol) and potassium fluoride (126 mg, 2.16 mmol) in THF (2.0 mL) and water (50 μL), stirred for 1 hour at room temperature. The crude product was re-crystallized from acetonitrile and ater (1:12 mL) to afford the title compound as a yellow solid (33.0 mg, 28%).

(ES,m/z): [M+H]⁺=537.25;

¹H NMR (400 MHz, DMSO-d₆) δ 10.65 (s, 1H), 10.14 (s, 1H), 9.44 (s, 1H), 9.19 (s, 1H), 8.24 (d, J=8.7 Hz, 2H), 7.69 (d, J=8.8 Hz, 2H), 7.29 (s, 1H), 5.11 (s, 1H), 4.15 (s, 1H), 3.01 (t, J=6.8 Hz, 2H), 2.68 (s, 3H), 2.37 (t, J=6.8 Hz, 2H), 2.13 (s, 6H), 1.91 (dd, J=12.5, 6.6 Hz, 2H), 1.85-1.73 (m, 2H), 1.69 (s, 2H), 1.57 (s, 2H).

### Example 85

### tert-Butyl 3-[(methanesulfonyloxy)methyl]azetidine-1-carboxylate

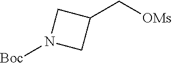 [SMILES: CC(C)(C)OC(=O)N1CC(COS(C)(=O)=O)C1]

A solution of tert-butyl 3-(hydroxymethyl)azetidine-1-carboxylate (20.0 g, 107 mmol, 1.0 eq.), methanesulfonyl chloride (13.5 g, 117 mmol, 1.1 eq.) and triethylamine (21.6 g, 213 mmol, 2.0 eq.) in dichloromethane (200 mL) was stirred overnight at room temperature. The resulting mixture was extracted with dichloromethane (3×200 mL). The combined organic layers were washed with brine (2×100 mL), dried (Na₂SO₄) and concentrated under reduced pressure to yield the title compound as a colourless oil (27.0 g, 95%).

### tert-Butyl 3-[(methylsulfanyl)methyl]azetidine-1-carboxylate

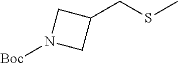

A solution of tert-butyl 3-[(methanesulfonyloxy)methyl]azetidine-1-carboxylate (27.0 g, 102 mmol, 1.0 eq.) and sodium methanethiolate (14.2 g, 203 mmol, 2.0 eq.) in DMF (270 mL) was stirred overnight at room temperature. The resulting mixture was extracted with ethyl acetate (2×200 mL). The combined organic layers were washed with brine (3×200 mL), dried (Na₂SO₄) and concentrated under reduced pressure. The residue was purified by reverse phase flash column chromatography eluting with acetonitrile (10-50%) in water (0.1% formic acid), to yield the title compound as a colourless oil (12.0 g, 54%). tert-Butyl 3-(methanesulfonylmethyl)azetidine-1-carboxylate

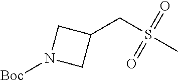 [SMILES: CC(C)(C)OC(=O)N1CC(CS(C)(=O)=O)C1]

To a stirred solution of tert-butyl 3-[(methylsulfanyl)methyl]azetidine-1-carboxylate (6.00 g, 27.6 mmol, 1.0 eq.) in dichloromethane (60 mL) was added m-CPBA (10.5 g, 60.7 mmol, 2.2 eq.) portionwise at 0° C. The resulting mixture was stirred for 2 hours at room temperature then diluted with dichloromethane (300 mL) and washed with saturated aqueous sodium hydrogen carbonate solution (2×200 mL). The combined organic layers were washed with brine (2×100 mL), dried (Na₂SO₄) and concentrated under reduced pressure to yield the title compound as a white solid (6.00 g, 87%).

### 3-Methansulfonylmethyl)azetidine hydrochloride

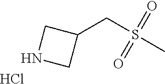

A solution of tert-butyl 3-(methanesulfonylmethyl)azetidine-1-carboxylate (6.00 g, 24.1 mmol, 1.0 eq.) in hydrochloric acid in methanol (50 mL) was stirred for 2 hours at room temperature. The resulting mixture was concentrated under reduced pressure to yield the title compound, which used in the next step without further purification.

### 3-(Methanesulfonylmethyl)-1-[(3-nitrophenyl)methyl]azetidine

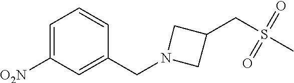 [SMILES: CS(=O)(=O)CC1CN(Cc2cccc([N+](=O)[O-])c2)C1]

A solution of 3-(methanesulfonylmethyl)azetidine (2.00 g, 13.4 mmol, 1.0 eq.), 1-(bromomethyl)-3-nitrobenzene (3.47 g, 16.1 mmol, 1.2 eq.) and cesium carbonate (13.1 g, 40.2 mmol, 3.0 eq.) in acetonitrile (200 mL) was stirred for 2 hours at 80° C. The residue was purified by flash column chromatography eluting with ethyl acetate (30%) in 40-60 petroleum ether to afford the title compound as a yellow solid (700 mg, 18%)

### 3-([3-(Methanesulfonylmethyl)azetidin-1-yl]methylaniline

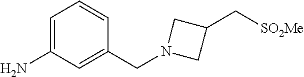 [SMILES: CS(=O)(=O)CC1CN(Cc2cccc(N)c2)C1]

General Procedure 5 was applied to 3-(methanesulfonylmethyl)-1-[(3-nitrophenyl)methyl]azetidine (700 mg, 2.46 mmol) and palladium on carbon (140 mg, 1.32 mmol) in THF (7 mL) to yield the title compound as a brown yellow solid (570 mg, 91%).

### 3-Cyclopentyl-1-(2-[(3-([3-(methanesulfonylmethyl)azetidin-1-yl]methyl)phenyl)amino]-5-[2-(triisopropylsilyl)ethynyl]pyrido[2,3-d]pyrimidin-7-ylurea

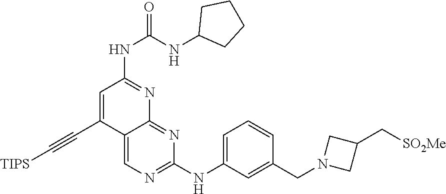

General Procedure 3 was applied to 3-cyclopentyl-1-(2-methanesulfonyl-5-[2-(triisopropylsilyl)ethynyl]pyrido[2,3-d]pyrimidin-7-ylurea (200 mg, 0.388 mmol), 3-([3-(methanesulfonylmethyl)azetidin-1-yl]methylaniline (148 mg, 0.582 mmol) and trifluoroacetic acid (57.6 μL, 0.776 mmol) in 2-butanol (2.0 mL), stirred overnight at 50° C. The residue was purified by reverse phase flash column chromatography eluting with acetonitrile (10-70%) in water (0.1% formic acid), to afford the title compound as a yellow solid (142 mg, 53%).

### 3-Cyclopentyl-1-(5-ethynyl-2-[(3-([3-(methanesulfonylmethyl)azetidin-1-yl]methyl)phenyl)amino]pyrido[2,3-d]pyrimidin-7-ylurea

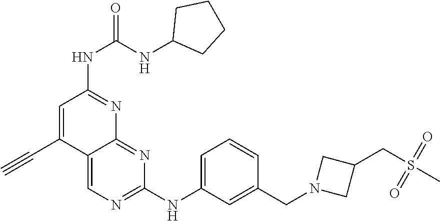 [SMILES: C#Cc1cc(NC(=O)NC2CCCC2)nc2nc(Nc3cccc(CN4CC(CS(C)(=O)=O)C4)c3)ncc12]

General Procedure 1 was applied to 3-cyclopentyl-1-(2-[(3-([3-(methanesulfonylmethyl)azetidin-1-yl]methyl)phenyl)amino]-5-[2-(triisopropylsilyl)ethynyl]pyrido[2,3-d]pyrimidin-7-ylurea (10.0 mg, 14.0 μmol), potassium fluoride (8.42 mg, 0.140 mmol), tetrahydrofuran (1.0 mL), DMF (1.0 mL) and water (10 uL), stirred for 1 hour at 60° C. The residue was purified by reverse phase flash column chromatography eluting with acetonitrile (10-65%) in water (0.1% ammonium acetate), to yield the title compound as a yellow solid (42.9 mg, 41%).

(ES,m/z): [M+H]⁺=534.30

¹H-NMR (400 MHz, DMSO-d₆) δ 10.13 (s, 1H), 10.05 (s, 1H), 9.47 (s, 1H), 9.11 (s, 1H), 8.00 (d, J=8.2 Hz, 1H), 7.78 (s, 1H), 7.24 (d, J=7.8 Hz, 1H), 7.20 (d, J=7.7 Hz, 1H), 6.93 (d, J=7.5 Hz, 1H), 5.06 (s, 1H), 4.12 (q, J=6.2 Hz, 1H), 3.54 (s, 2H), 3.46-3.36 (m, 4H), 2.93 (d, J=4.9 Hz, 5H), 2.84 (p, J=7.0 Hz, 1H), 1.94 (dt, J=12.9, 6.6 Hz, 2H), 1.77 (q, J=10.3, 7.5 Hz, 2H), 1.68 (q, J=8.5, 5.8 Hz, 2H), 1.61-1.48 (m, 2H).

### Example 86 tert-Butyl 3-([(4-methylbenzenesulfonyl)oxy]methylpyrrolidine-1-carboxylate

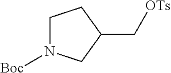 [SMILES: Cc1ccc(S(=O)(=O)OCC2CCN(C(=O)OC(C)(C)C)C2)cc1]

A solution of tert-butyl 3-(hydroxymethyl)pyrrolidine-1-carboxylate (10.0 g, 49.7 mmol, 1.0 eq.) and 4-toluenesulfonyl chloride (11.4 g, 59.6 mmol, 1.2 eq.) in dichloromethane (100 mL) was stirred for 16 hours at room temperature. The resulting mixture was washed with saturated aqueous sodium hydrogen carbonate solution (2×200 mL). The combined organic layers were washed with brine (2×200 mL), dried (Na₂SO₄) and concentrated under reduced pressure to yield the title compound as a colourless oil (10.0 g, 56%). tert-Butyl 3-(sulfanylmethyl)pyrrolidine-1-carboxylate

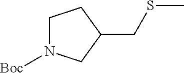

A solution of tert-butyl 3-[(methanesulfonyloxy)methyl]pyrrolidine-1-carboxylate (2.00 g, 7.16 mmol, 1.0 eq.) and (methylsulfanyl)sodium (1.00 g, 14.3 mmol, 2.0 eq.) in methanol (20 mL) was stirred for 16 hours at room temperature. The resulting mixture was extracted with ethyl acetate (2×50 mL). The combined organic layers were washed with brine (2×50 mL), dried (Na₂SO₄) and concentrated under reduced pressure. The residue was purified by flash column chromatography eluting with ethyl acetate (10%) in 40-60 petroleum ether afford the title compound as a colorless oil (1.20 g, 77%).

### tert-Butyl 3-(methanesulfonylmethyl)pyrrolidine-1-carboxylate

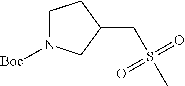 [SMILES: CC(C)(C)OC(=O)N1CCC(CS(C)(=O)=O)C1]

A mixture of tert-butyl 3-[(methylsulfanyl)methyl]pyrrolidine-1-carboxylate (6.00 g, 25.9 mmol, 1.0 eq.) and oxone (17.4 g, 103 mmol, 4.0 eq.) in THF (60 mL) and water (60 mL) was stirred for 3 hours at room temperature. The resulting mixture was extracted with ethyl acetate (3×500 mL). The combined organic layers were washed with brine (2×200 mL), dried (Na₂SO₄) and concentrated under reduced pressure to yield the title compound as a colourless oil (6.00 g, 88%).

### 3-Methanesuifonylmethyl)pyrroiidine

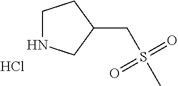

A solution of tert-butyl 3-(methanesulfonylmethyl)pyrrolidine-1-carboxylate (6.00 g, 22.8 mmol, 1.0 eq.) in hydrochloric acid in methanol (60 mL) was stirred for 2 hours at room temperature. The resulting mixture was concentrated under reduced pressure to yield the title compound as a colourless oil (4.00 g), used in the next step without further purification.

### 3-(Methanesulfonylmethyl)-1-[(3-nitrophenyl)methyl]pyrrolidine

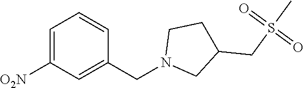 [SMILES: CS(=O)(=O)CC1CCN(Cc2cccc([N+](=O)[O-])c2)C1]

A solution of 3-(methanesulfonylmethyl)pyrrolidine (600 mg, 3.67 mmol, 1.0 eq.), cesium carbonate (2.39 g, 7.35 mmol, 2.0 eq.) and 3-nitrobenzaldehyde (666 mg, 4.41 mmol, 1.2 eq.) in acetonitrile (6.0 ml) was stirred for 2 hours at 80° C. The residue was purified by flash column chromatography eluting with ethyl acetate (50%) in 40-60 petroleum ether to afford the title compound as a yellow solid (600 mg, 55%).

### 3-([3-(Methanesulfonylmethyl)pyrrolidin-1-yl]methylaniline

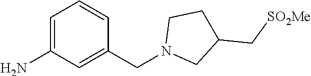 [SMILES: CS(=O)(=O)CC1CCN(Cc2cccc(N)c2)C1]

General Procedure 5 was applied to 3-(methanesulfonylmethyl)-1-[(3-nitrophenyl)methyl]pyrrolidine (600 mg, 2.01 mmol) and palladium on carbon (120 mg, 1.13 mmol) in methanol (30 mL) to yield the title compound as a white solid (500 mg, 92%).

### 3-Cyclopentyl-1-(2-[(3-([3-(methanesulfonylmethyl)pyrrolidin-1-yl]methylphenyl)amino]-5-[2-(triisopropylsilyl)ethynyl]pyrido[2,3-d]pyrimidin-7-ylurea

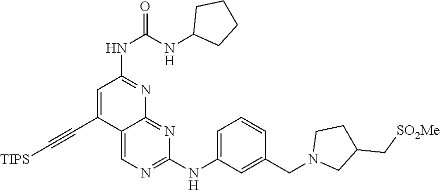

General Procedure 3 was applied to 3-cyclopentyl-1-(2-methanesulfinyl-5-[2-(triisopropylsilyl)ethynyl]pyrido[2,3-d]pyrimidin-7-ylurea (300 mg, 0.600 mmol), 3-([3-(methanesulfonylmethyl)pyrrolidin-1-yl]methylaniline (193 mg, 0.720 mmol) and trifluoroacetic acid (137 mg, 1.20 mmol) in butan-2-ol (3.0 mL), stirred for 14 hours at 100° C. The residue was purified by reverse phase flash column chromatography eluting with acetonitrile (50-100%) in water (0.1% trifluoroacetic acid) to yield the title compound as a yellow solid (220 mg, 52%).

### 3-Cyclopentyl-1-(5-ethynyl-2-[(3-([3-(methanesulfonylmethyl)pyrrolidin-1-yl]methylphenyl)amino]pyrido[2,3-d]pyrimidin-7-ylurea

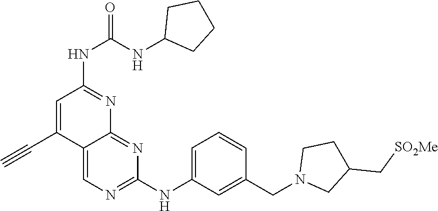 [SMILES: C#Cc1cc(NC(=O)NC2CCCC2)nc2nc(Nc3cccc(CN4CCC(CS(C)(=O)=O)C4)c3)ncc12]

General Procedure 1 was applied to 3-cyclopentyl-1-(2-[(3-([3-(methanesulfonylmethyl)pyrrolidin-1-yl]methyl)phenyl)amino]-5-[2-(triisopropylsilyl)ethynyl]pyrido[2,3-d]pyrimidin-7-ylurea (200 mg, 0.284 mmol), potassium fluoride (165 mg, 2.84 mmol) in water (50 uL) and DMF (2.0 mL), stirred for 2 hours at 50° C. The crude product was purified by Prep-HPLC eluting with acetonitrile (15-40%) in water (0.1% formic acid), to afford the title compound as a yellow solid (39.0 mg, 25%).

(ES,m/z): [M+H]⁺=548.15;

¹H-NMR (400 MHz, DMSO-d₆) δ 10.15 (s, 1H), 10.07 (s, 1H), 9.49 (s, 1H), 9.11 (s, 1H), 8.02 (d, J=8.1 Hz, 1H), 7.81 (t, J=1.9 Hz, 1H), 7.25 (t, J=7.8 Hz, 1H), 7.18 (s, 1H), 6.99 (d, J=7.5 Hz, 1H), 5.08 (s, 1H), 4.11 (h, J=6.1 Hz, 1H), 3.64-3.52 (m, 2H), 3.23 (dd, J=7.1, 4.2 Hz, 2H), 2.95 (s, 3H), 2.77 (dd, J=9.2, 7.3 Hz, 1H), 2.59 (dt, J=14.7, 6.9 Hz, 2H), 2.46 (t, J=7.2 Hz, 1H), 2.28 (dd, J=9.3, 6.7 Hz, 1H), 2.05 (dq, J=13.3, 7.6, 6.4 Hz, 1H), 1.94 (dq, J=13.4, 7.0 Hz, 2H), 1.79 (q, J=6.7, 5.3 Hz, 2H), 1.72-1.62 (m, 2H), 1.59-1.51 (m, 3H).

### Example 87

### tert-Butyl 4-([(4-methylbenzenesulfonyl)oxy]methylpiperidine-1-carboxylate

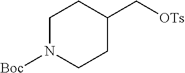 [SMILES: Cc1ccc(S(=O)(=O)OCC2CCN(C(=O)OC(C)(C)C)CC2)cc1]

To a stirred solution of tert-butyl 4-(hydroxymethyl)piperidine-1-carboxylate (20.0 g, 92.9 mmol, 1.0 eq.) in dichloromethane (200 mL) was added triethylamine (32.0 mL, 230 mmol, 2.5 eq.) and toluenesulfonyl chloride (35.4 g, 186 mmol, 2.0 eq.) dropwise at room temperature. The reaction mixture was irradiated with microwave radiation overnight at room temperature. The residue was purified by flash column chromatography eluting with ethyl acetate (12%) in 40-60 petroleum ether to afford the title compound as a yellow oil (27.0 g, 79%).

### tert-Butyl 4-[(methylsulfanyl)methyl]piperidine-1-carboxylate

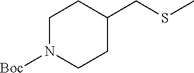 [SMILES: CSCC1CCN(C(=O)OC(C)(C)C)CC1]

To a stirred solution of tert-butyl 4-[(methanesulfonyloxy)methyl]piperidine-1-carboxylate (27.0 g, 92.0 mmol, 1.0 eq.) in DMF (71 ml) was added sodium methanethiolate (19.4 g, 276 mmol, 3.0 eq.) at room temperature. The resulting mixture was stirred overnight at room temperature then extracted with ethyl acetate (3×200 mL). The combined organic layers were washed with brine (2×250 mL), dried (Na₂SO₄) and concentrated under reduced pressure. The residue was purified by flash column chromatography eluting with ethyl acetate (20-25%) in 40-60 petroleum ether to afford the title compound as a yellow oil (16.4 g, 72%).

### tert-Butyl 4-(methanesulfonylmethyl)piperidine-1-carboxylate

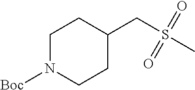 [SMILES: CC(C)(C)OC(=O)N1CCC(CS(C)(=O)=O)CC1]

To a stirred solution of tert-butyl 4-[(methylsulfanyl)methyl]piperidine-1-carboxylate (10.5 g, 42.8 mmol, 1.0 eq.) in dichloromethane (105 mL) added m-CPBA (18.5 g, 107 mmol, 2.5 eq.) at 0-5° C. The resulting mixture was stirred for 30 minutes at room temperature then washed with saturated aqueous sodium hydrogen carbonate solution (400 mL). The aqueous layer was extracted with dichloromethane (3×150 mL), the combined organic layers were washed with brine (400 mL), dried (Na₂SO₄) and concentrated under reduced pressure. The residue was purified by flash column chromatography eluting with ethyl acetate (25-35%) in 40-60 petroleum ether to afford as a white solid (8.78 g, 78%).

### 4-(Methanesulfonylmethyl)piperidine

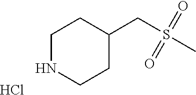

A solution of tert-butyl 4-(methanesulfonylmethyl)piperidine-1-carboxylate (8.80 g, 31.7 mmol, 1.0 eq.) and hydrochloric acid in methanol (88 mL, 4M) was stirred for 4 hours at room temperature. The resulting mixture was concentrated under reduced pressure, to afford the title compound as a white solid (8.39 g, 149%) used in the next step without further purification.

### 4-(Methanesulfonylmethyl)-1-[(3-nitrophenyl)methyl]piperidine

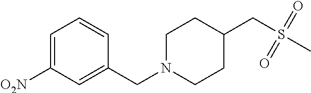 [SMILES: CS(=O)(=O)CC1CCN(Cc2cccc([N+](=O)[O-])c2)CC1]

A solution of 4-(methanesulfonylmethyl)piperidine (2.00 g, 11.3 mmol, 1.0 eq.), 3-(bromomethyl)benzaldehyde (2.47 g, 12.4 mmol, 1.1 eq.) and cesium carbonate (11.0 g, 33.8 mmol, 3.0 eq.) in acetonitrile (20 mL) was stirred for 2 hours at 50° C. then concentrated under reduced pressure. The residue was purified by flash column chromatography eluting with ethyl acetate (30%) in 40-60 petroleum ether to afford the title compound asa brown oil (1.89 g, 54%)

### 3-([4-(Methanesulfonylmethyl)piperidin-1-yl]methylaniline

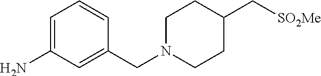 [SMILES: CS(=O)(=O)CC1CCN(Cc2cccc(N)c2)CC1]

General Procedure 5 was applied to 4-(methanesulfonylmethyl)-1-[(3-nitrophenyl)methyl]piperidine (1.80 g, 5.76 mmol) and palladium on carbon (1.22 g, 1.15 mmol) in methanol (30 mL) to afford the title compound as a brown oil (1.49 g, 92%).

### 3-Cyclopentyl-1-(2-[(3-([4-(methanesulfonylmethyl)piperidin-1-yl]methylphenyl)amino]-5-[2-(triisopropylsilyl)ethynyl]pyrido[2,3-d]pyrimidin-7-ylurea

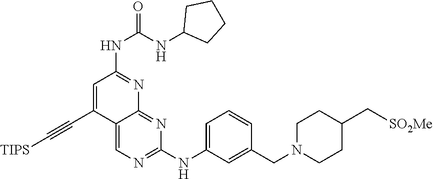

General Procedure 3 was applied to 3-cyclopentyl-1-(2-methanesulfonyl-5-[2-(triisopropylsilyl)ethynyl]pyrido[2,3-d]pyrimidin-7-ylurea (300 mg, 0.582 mmol), 3-([4-(methanesulfonylmethyl)piperidin-1-yl]methylaniline (246 mg, 0.873 mmol) and trifluoroacetic acid (86.4 uL, 1.16 mmol) and 2-butanol (3 mL), stirred overnight at 100° C. The residue was purified by reverse phase flash column chromatography eluting with acetonitrile (10-85%) in water (0.1% trifluoroacetic acid), to yield the title compound as a brown yellow solid (365 mg, 87%).

### 3-Cyclopentyl-1-(5-ethynyl-2-[(3-([4-(methanesulfonylmethyl)piperidin-1-yl]methylphenyl)amino]pyrido[2,3-d]pyrimidin-7-viurea

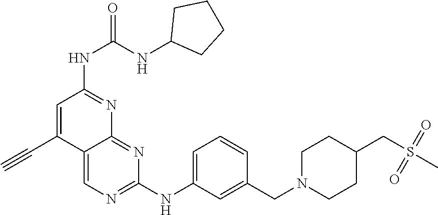 [SMILES: C#Cc1cc(NC(=O)NC2CCCC2)nc2nc(Nc3cccc(CN4CCC(CS(C)(=O)=O)CC4)c3)ncc12]

General Procedure 1 was applied to 3-cyclopentyl-1-(2-[(3-([4-(methanesulfonylmethyl)piperidin-1-yl]methyl)phenyl)amino]-5-[2-(triisopropylsilyl)ethynyl]pyrido[2,3-d]pyrimidin-7-ylurea (150 mg, 0.209 mmol) and potassium fluoride (121 mg, 2.09 mmol) in THE (1.5 mL), DMF (1.5 mL) and water (150 uL), stirred for 1 hour at 60° C. The residue was purified by reverse phase flash column chromatography eluting with acetonitrile (10-50%) in water (0.1% formic acid), to yield the title compound as a yellow solid (79.6 mg, 68%).

(ES,m/z): [M+H]⁺=562.25

¹H NMR (400 MHz, DMSO-d₆) δ 10.14 (s, 1H), 10.07 (s, 1H), 9.50 (s, 1H), 9.11 (s, 1H), 8.05-7.97 (m, 1H), 7.79 (d, J=2.2 Hz, 1H), 7.25 (t, J=7.9 Hz, 1H), 7.17 (s, 1H), 6.98 (d, J=7.8 Hz, 1H), 5.08 (s, 1H), 4.12 (hept, J=6.1, 5.5 Hz, 1H), 3.44 (s, 2H), 3.09 (d, J=6.3 Hz, 2H), 2.97 (s, 3H), 2.80 (d, J=11.1 Hz, 2H), 2.02-1.87 (m, 5H), 1.86-1.74 (m, 4H), 1.73-1.61 (m, 2H), 1.55 (dd, J=12.1, 6.5 Hz, 2H), 1.34 (q, J=11.0 Hz, 2H).

### Example 88

### 5-Nitro-1,2,3,4-tetrahydroisoquinoline

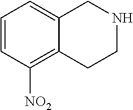 [SMILES: O=[N+]([O-])c1cccc2c1CCNC2]

A solution of 5-nitroisoquinoline (10.0 g, 57.4 mmol, 1.0 eq.) and sodium borohydride (21.7 g, 574 mmol, 10 eq.) in acetic acid (98 mL) was stirred for 5 minutes at 0° C. then for 15 minutes at room temperature. The mixture was basified to pH 9 with sodium hydroxide. The aqueous layer was extracted with ethyl acetate (100 mL) then washed with water (5×100 mL) and concentrated under reduced pressure to yield the title compound as a black green solid (6.50 g, 63%).

### 2-Methyl-5-nitro-3,4-dihydro-1H-isoquinoline

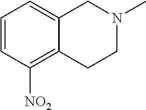

A solution of 5-nitro-1,2,3,4-tetrahydroisoquinoline (1.30 g, 7.29 mmol, 1.0 eq.), formaldehyde solution (2.19 g, 72.9 mmol, 10 eq.), acetic acid (40.0 mg, 0.730 mmol, 0.10 eq.) and sodium borohydride (1.38 g, 36.5 mmol, 5 eq.) in methanol (20 mL) was stirred for 30 minutes at room temperature. The resulting mixture was diluted with water (100 mL) and extracted with ethyl acetate (100 mL). The organic layer was concentrated under reduced pressure to yield the title compound as a black solid (865 mg, 61%).

### 2-Methyl-3,4-dihydro-1H-isoquinolin-5-amine

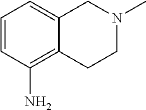

General Procedure 5 was applied to 2-methyl-5-nitro-3,4-dihydro-1H-isoquinoline (860 mg, 4.47 mmol) and palladium on carbon (230 mg, 0.216 mmol) in dichloromethane (20 mL) to yield the title compound as a green solid (624 mg, 86%).

### 3-Cyclopentyl-1-{2-[(2-methyl-3,4-dihydro-1H-isoquinolin-5-yl)amino]-5-[2-(triisopropylsilyl)ethynyl]pyrido[2,3-d]pyrimidin-7-yl}urea

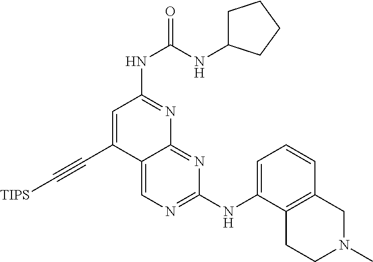

General Procedure 3 was applied top 3-cyclopentyl-1-{2-methanesulfinyl-5-[2-(triisopropylsilyl)ethynyl]pyrido[2,3-d]pyrimidin-7-yl}urea (220 mg, 0.440 mmol), 2-methyl-3,4-dihydro-1H-isoquinolin-5-amine (85.7 mg, 0.528 mmol) and trifluoroacetic acid (70.0 μL, 0.880 mmol) in 2-methyl-2-butanol (5.0 mL), stirred for 1 hour at 80° C. The residue was purified by reverse phase flash column chromatography eluting with acetonitrile (10-100%) in water (0.1% formic acid),to yield the title compound as a light yellow solid (120 mg, 45%).

### 3-Cyclopentyl-1-{5-ethynyl-2-[(2-methyl-3,4-dihydro-1H-isoquinolin-5-yl)amino]pyrido[2,3-d]pyrimidin-7-yl}urea

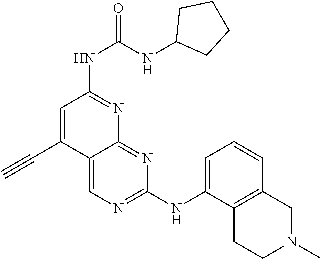 [SMILES: C#Cc1cc(NC(=O)NC2CCCC2)nc2nc(Nc3cccc4c3CCN(C)C4)ncc12]

General Procedure 1 was applied to 3-cyclopentyl-1-{2-[(2-methyl-3,4-dihydro-1H-isoquinolin-5-yl)amino]-5-[2-(triisopropylsilyl)ethynyl]pyrido[2,3-d]pyrimidin-7-yl}urea (100 mg, 0.167 mmol.) and potassium fluoride (48.6 mg, 0.835 mmol) in DMF (1.0 mL), THF (1.0 mL), stirred for 30 minutes at room temperature. The residue was purified by reverse phase flash column chromatography eluting with acetonitrile (10-70%) in water (0.1% formic acid), to yield the title compound as a light yellow solid (26.0 mg, 35%).

(ES, m/z) [M+H]⁺=442.10

¹H-NMR (300 MHz, DMSO-d₆) δ 9.95 (s, 1H), 9.25 (d, J=19.0 Hz, 2H), 9.05 (s, 1H), 7.54 (d, J=7.9 Hz, 1H), 7.22-7.04 (m, 2H), 6.90 (d, J=7.5 Hz, 1H), 5.04 (s, 1H), 4.07 (q, J=6.0 Hz, 1H), 3.49 (s, 2H), 2.78 (d, J=6.1 Hz, 2H), 2.56 (t, J=5.9 Hz, 2H), 2.33 (s, 3H), 1.86 (dd, J=12.1, 6.0 Hz, 2H), 1.62 (dt, J=21.4, 7.9 Hz, 4H), 1.45 (dd, J=11.6, 6.0 Hz, 2H).

### Example 89

### 3-(Methanesulfonylmethyl)-1-(2-nitrophenyl)pyrrolidine

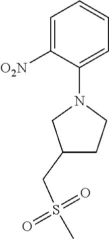 [SMILES: CS(=O)(=O)CC1CCN(c2ccccc2[N+](=O)[O-])C1]

A solution of 3-(methanesulfonylmethyl)pyrrolidine (1.00 g, 6.13 mmol, 1.0 eq.), o-fluoronitrobenzene (860 mg, 6.13 mmol, 1.0 eq.) and potassium carbonate (1.69 g, 12.3 mmol, 2.0 eq.) in acetonitrile (10 mL) was stirred for 2 hours at 80° C. The residue was purified by flash column chromatography eluting with ethyl acetate (35%) in 40-60 petroleum ether to afford the title compound as a yellow solid (1.10 g, 63%).

### 2-[3-(Methanesulfonylmethyl)pyrrolidin-1-yl]aniline

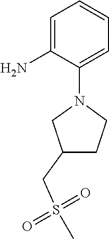 [SMILES: CS(=O)(=O)CC1CCN(c2ccccc2N)C1]

General Procedure 5 was applied to 3-(methanesulfonylmethyl)-1-(2-nitrophenyl)pyrrolidine (1.00 g, 3.52 mmol) and palladium on carbon (200 mg, 1.88 mmol) in methanol (10 mL) to yield the title compound as a yellow solid (850 mg, 95%).

### 3-Cyclopentyl-1-[2-((2-[3-(methanesulfonylmethyl)pyrrolidin-1-yl]phenylamino)-5-[2-(triisopropyisilyl)ethynyl]pyrido[2,3-d]pyrimidin-7-yl]urea

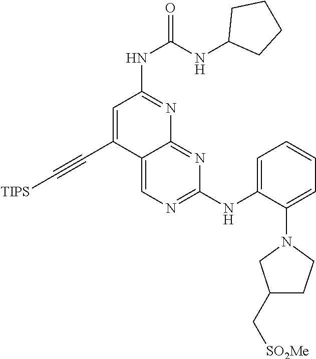

General Procedure 3 was applied to 2-[3-(methanesulfonylmethyl)pyrrolidin-1-yl]aniline (183 mg, 0.722 mmol), 3-cyclopentyl-1-(2-methanesulfinyl-5-[2-(triisopropylsilyl)ethynyl]pyrido[2,3-d]pyrimidin-7-ylurea (300 mg, 0.602 mmol) and trifluoroacetic acid (137 mg, 1.20 mmol) in 2-butanol (3.0 mL), stirred for 4 hours at 80° C. The residue was purified by reverse phase flash column chromatography eluting with acetonitrile (30-100%) in water (0.1% trifluoroacetic acid), to yield the title compound as a yellow solid (250 mg, 60%).

### 3-Cyclopentyl-1-[5-ethynyl-2-((2-[3-(methanesulfonylmethyl)pyrrolidin-1-yl]phenylamino)pyrido[2,3-d]pyrimidin-7-yl]urea

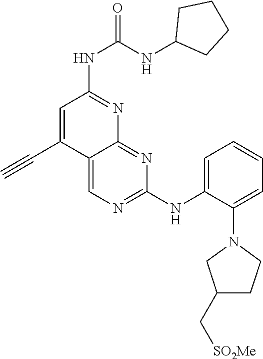 [SMILES: C#Cc1cc(NC(=O)NC2CCCC2)nc2nc(Nc3ccccc3N3CCC(CS(C)(=O)=O)C3)ncc12]

General Procedure 1 was applied to 3-cyclopentyl-1-[2-((2-[3-(methanesulfonylmethyl)pyrrolidin-1-yl]phenylamino)-5-[2-(triisopropylsilyl)ethynyl]pyrido[2,3-d]pyrimidin-7-yl]urea (200 mg, 0.290 mmol), potassium fluoride (168 mg, 2.90 mmol) in water (20 uL) and DMF (2.0 mL), stirred for 2 hours at 50° C. The crude product was purified by Prep-HPLC eluting with acetonitrile (45-70%) in water (0.1% formic acid), to afford the title compound as a yellow solid (90.9 mg, 58%).

(ES,m/z): [M+H]⁺=534.15;

¹H NMR (400 MHz, DMSO-d₆): δ 9.93 (s, 1H), 9.33 (s, 1H), 9.05 (s, 1H), 8.96 (s, 1H), 7.20 (s, 1H), 7.87 (s, 1H), 7.08 (td, J=7.7, 7.1, 1.6 Hz, 1H), 6.99 (d, J=8.0 Hz, 1H), 6.89 (t, J=7.5 Hz, 1H), 5.04 (s, 1H), 4.13-4.02 (m, 1H), 3.36 (d, J=7.3 Hz, 1H), 3.33 (s, 2H), 3.24-3.15 (m, 2H), 3.05 (dd, J=9.5, 6.9 Hz, 1H), 2.98 (s, 3H), 2.72 (q, J=7.1 Hz, 1H), 2.16 (dq, J=12.4, 6.8 Hz, 1H), 1.86 (dt, J=12.9, 6.6 Hz, 2H), 1.74 (dq, J=13.2, 7.3 Hz, 3H), 1.62 (d, J=5.9 Hz, 2H), 1.49 (td, J=9.0, 6.5, 3.5 Hz, 2H).

### Example 90

### 2-(4-Methanesulfonylpiperidin-1-yl)aniline

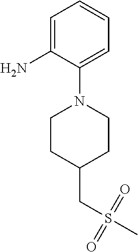 [SMILES: CS(=O)(=O)CC1CCN(c2ccccc2N)CC1]

General Procedure 5 was applied to 4-methanesulfonyl-1-(2-nitrophenyl)piperidine (1.80 g, 6.33 mmol) and palladium on carbon (135 mg, 1.27 mmol) in methanol (30 mL) to yield the title compound as a brown red solid (1.33 g, 82%).

### 3-Cyclopentyl-1-[2-({2-[4-(methanesulfonylmethyl)piperidin-1-yl]phenyl}amino)-5-[2-(triisopropylsilyl)ethynyl]pyrido[2,3-d]pyrimidin-7-yl]urea

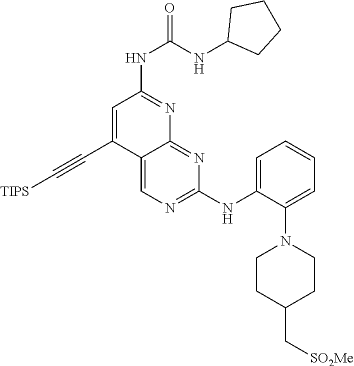

General Procedure 3 was applied to 3-cyclopentyl-1-{2-methanesulfonyl-5-[2-(triisopropylsilyl)ethynyl]pyrido[2,3-d]pyrimidin-7-yl}urea (300 mg, 0.582 mmol), 2-[4-(methanesulfonylmethyl)piperidin-1-yl]aniline (187 mg, 0.698 mmol) and trifluoroacetic acid (86.4 uL, 1.16 mmol) and 2-butanol (3.0 mL), stirred for 1 hour at 100° C. The residue was purified by reverse phase flash column chromatography eluting with acetonitrile (10-100%) in water (0.1% formic acid), to afford the title compound as a brown yellow solid (421 mg, 102%).

### 3-Cyclopentyl-1-[5-ethynyl-2-({2-[4-(methanesulfonylmethyl)piperidin-1-yl]phenyl}amino)pyrido[2,3-d]pyrimidin-7-yl]urea

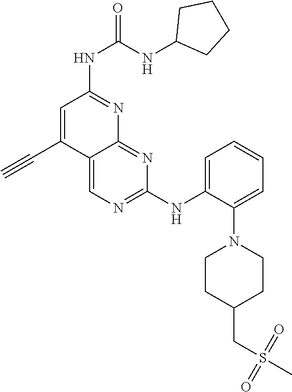 [SMILES: C#Cc1cc(NC(=O)NC2CCCC2)nc2nc(Nc3ccccc3N3CCC(CS(C)(=O)=O)CC3)ncc12]

General Procedure 1 was applied to 3-cyclopentyl-1-[2-({2-[4-(methanesulfonylmethyl)piperidin-1-yl]phenyl}amino)-5-[2-(triisopropylsilyl)ethynyl]pyrido[2,3-d]pyrimidin-7-yl]urea (150 mg, 0.213 mmol) and potassium fluoride (124 mg, 2.13 mmol) in THF (1.5 mL), DMF (1.5 mL) and water (150 uL), stirred for 1 hour at 60° C. The residue was purified by reverse phase flash column chromatography eluting with acetonitrile (10-70%) in water (0.1% formic acid). The precipitated solids were collected by filtration and washed with acetonitrile (3×3 mL) and water (3×3 mL) to yield the title compound as a yellow solid (23.0 mg, 19%).

(ES,m/z): [M+H]⁺=548.15

¹H NMR (400 MHz, DMSO-d₆) δ 10.10 (s, 1H), 9.56 (s, 1H), 9.12 (s, 1H), 8.76 (d, J=8.0 Hz, 1H), 8.69 (s, 1H), 7.29 (dd, J=7.8, 1.6 Hz, 1H), 7.22 (s, 1H), 7.12 (td, J=7.6, 1.6 Hz, 1H), 7.06 (td, J=7.5, 1.7 Hz, 1H), 5.08 (s, 1H), 4.16 (s, 1H), 3.24 (d, J=6.4 Hz, 2H), 3.02 (s, 3H), 2.98 (d, J=11.7 Hz, 2H), 2.82-2.70 (m, 2H), 2.13 (s, 1H), 2.02 (d, J=12.8 Hz, 2H), 1.93-1.80 (m, 4H), 1.72 (d, J=7.1 Hz, 2H), 1.59 (d, J=11.3 Hz, 4H).

### Example 91

### Benzyl N-[(1-{[2-(methylsulfanyl)-5-[2-(triisopropylsilyl)ethynyl]pyrido[2,3-d]pyrimidin-7-yl]amino}cyclopentyl)methyl]carbamate

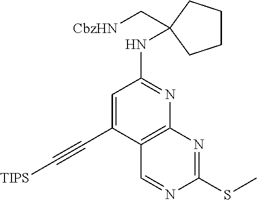

A solution of 7-chloro-2-(methylsulfanyl)-5-[2-(triisopropylsilyl)ethynyl]pyrido[2,3-d]pyrimidine (15.0 g, 38.2 mmol, 1.0 eq.), benzyl N-[(1-aminocyclopentyl)methyl]carbamate (10.5 g, 42.1 mmol, 1.1 eq.), copper iodide (730 g, 3.83 mmol, 0.1 eq.) and potassium carbonate (10.6 g, 76.5 mmol, 2.0 eq.) in DMF (120 mL) was stirred for 3 hours at 100° C. The mixture was allowed to cool down to room temperature then diluted with water (100 mL). The aqueous layer was extracted with ethyl acetate (2×50 mL). The residue was purified by flash column chromatography eluting with ethyl acetate (25%) in 40-60 petroleum ether to afford the title compound as a light pink solid (8.60 g, 37%).

### N-[1-(Aminomethyl)cyclopentyl]-2-(methylsulfanyl)-5-[2-(triisopropylsilyl)ethynyl]pyrido[2,3-d]pyrimidin-7-amine

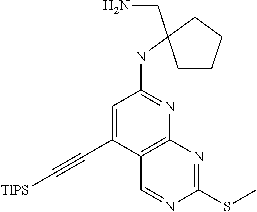

A solution of benzyl N-[(1-{[2-(methylsulfanyl)-5-[2-(triisopropylsilyl)ethynyl]pyrido[2,3-d]pyrimidin-7-yl]amino}cyclopentyl)methyl]carbamate (2.00 g, 3.31 mmol, 1.0 eq.) and hydrogen bromide in acetic acid (4.84 mL, 165 mmol, 50 eq.) was stirred for 3 hours at room temperature. The resulting mixture was concentrated under reduced pressure to yield the title compound as a yellow green solid (1.20 g, 77%).

### 1-[2-(Methylsulfanyl)-5-[2-(triisopropylsilyl)ethynyl]pyrido[2,3-d]pyrimidin-7-yl]-1,3-diazaspiro[4,4]nonan-2-one

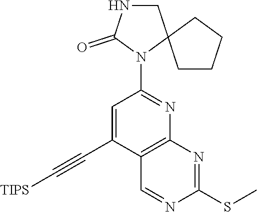

A solution of N-[1-(aminomethyl)cyclopentyl]-2-(methylsulfanyl)-5-[2-(triisopropylsilyl)ethynyl]pyrido [2,3-d]pyrimidin-7-amine (1.00 g, 2.13 mmol, 1.0 eq.), CDI (520 mg, 3.19 mmol, 1.5 eq.) and diisopropylethylamine (550 g, 4.26 mmol, 2.0 eq.) in DMF (10 mL) was stirred for 1 hour at 60° C. The mixture was allowed to cool down to room temperature. The residue was purified by flash column chromatography eluting with ethyl acetate (16%) in 40-60 petroleum ether to afford the title compound as a reddish brown yellow solid (600 mg, 57%).

### 1-{2-Methanesulfinyl-5-[2-(triisopropylsilyl)ethynyl]pyrido[2,3-d]pyrimidin-7-yl}-1,3-diazaspiro[4.4]nonan-2-one

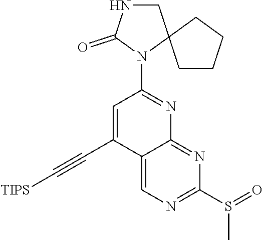

General Procedure 2 was applied to 1-[2-(methylsulfanyl)-5-[2-(triisopropylsilyl)ethynyl]pyrido[2,3-d]pyrimidin-7-yl]-1,3-diazaspiro[4.4]nonan-2-one (1.00 g, 2.02 mmol) and m-CPBA (348 mg, 2.02 mmol) in dichloromethane (2.0 mL). The residue was purified by flash column chromatography eluting with ethyl acetate (16%) in 40-60 petroleum ether to afford the title compound as a reddish brown yellow solid (860 mg, 83%).

### 1-(2-{[4-(Morpholin-4-yl)phenyl]amino}-5-[2-(triisopropylsilyl)ethynyl]pyrido[2,3-d]pyrimidin-7-yl)-1,3-diazaspiro[4.4]nonan-2-one

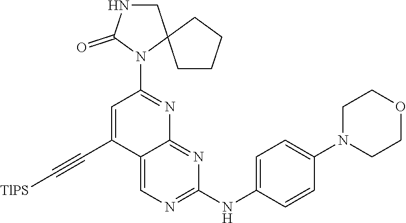

General Procedure 3 was applied to 1-{2-methanesulfinyl-5-[2-(triisopropylsilyl)ethynyl]pyrido[2,3-d]pyrimidin-7-yl}-1,3-diazaspiro[4.4]nonan-2-one (300 mg, 0.586 mmol), 4-(morpholin-4-yl)aniline (125 mg, 0.703 mmol) and trifluoroacetic acid (90.0 μL) in 2-methyl-2-butanol (2.0 mL), stirred for 1 hour at 100° C. The residue was purified by reverse phase flash column chromatography eluting with acetonitrile (10-80%) in water (0.1% formic acid), to yield the title compound as a yellow solid (143 mg, 39%).

### 1-(5-Ethynyl-2-{[4-(morpholin-4-yl)phenyl]amino}pyrido[2,3-d]pyrimidin-7-yl)-1,3-diazaspiro[4.4]nonan-2-one

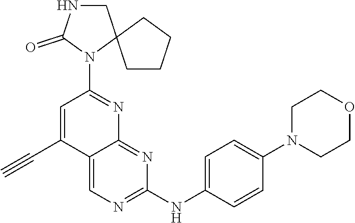 [SMILES: C#Cc1cc(N2C(=O)NCC23CCCC3)nc2nc(Nc3ccc(N4CCOCC4)cc3)ncc12]

General Procedure 1 was applied to 1-(2-{[4-(morpholin-4-yl)phenyl]amino}-5-[2-(triisopropylsilyl)ethynyl]pyrido[2,3-d]pyrimidin-7-yl)-1,3-diazaspiro[4.4]nonan-2-one (100 mg, 0.160 mmol) and potassium fluoride (92.8 mg, 1.60 mmol) in THE (2.0 mL), DMF (2.0 mL) and water (0.50 mL), stirred for 1 hour at 60° C. The residue was purified by reverse phase flash column chromatography eluting with acetonitrile (10-50%) in water (0.1% formic acid), to yield the title compound as an orange solid (12.0 mg, 16%).

(ES, m/z) [M+H]⁺=470.10

¹H-NMR (300 MHz, DMSO-d₆) δ 9.92 (s, 1H), 9.12 (s, 1H), 8.23 (s, 1H), 7.85 (s, 2H), 7.73 (s, 1H), 6.92 (d, J=9.1 Hz, 2H), 4.99 (s, 1H), 3.79-3.70 (m, 4H), 3.06 (t, J=4.8 Hz, 4H), 2.63 (s, 2H), 1.66 (s, 4H).

### Example 92

### 1-[2-({4-[4-(Oxetan-3-yl)piperazin-1-yl]phenyl}amino)-5-[2-(triisopropylsilyl)ethynyl]pyrido[2,3-d]pyrimidin-7-yl]-1,3-diazaspiro[4.4]nonan-2-one

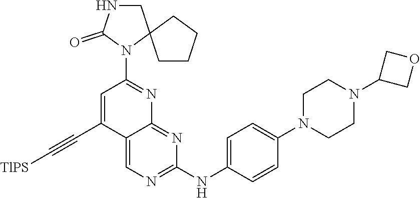

General Procedure 3 was applied to 1-{2-methanesulfinyl-5-[2-(triisopropylsilyl)ethynyl]pyrido[2,3-d]pyrimidin-7-yl}-1,3-diazaspiro[4.4]nonan-2-one (300 mg, 0.586 mmol), 4-[4-(oxetan-3-yl)piperazin-1-yl]aniline (150 mg, 0.645 mmol) and trifluoroacetic acid (90.0 μL, 1.17 mmol) in 2-methyl-2-butanol (2.0 mL), stirred for 1 hour at 100° C. The residue was purified by reverse phase flash column chromatography eluting with acetonitrile (10-80%) in water (0.1% formic acid), to yield the title compound as a yellow solid (123 mg, 31%).

### 1-[5-Ethynyl-2-({4-[4-(oxetan-3-yl)piperazin-1-yl]phenyl}amino)pyrido[2,3-d]pyrimidin-7-yl]-1,3-diazaspiro[4.4]nonan-2-one

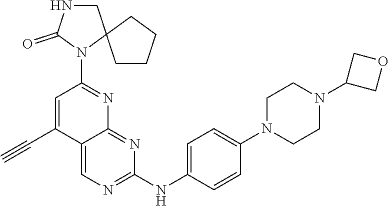 [SMILES: C#Cc1cc(N2C(=O)NCC23CCCC3)nc2nc(Nc3ccc(N4CCN(C5COC5)CC4)cc3)ncc12]

General Procedure 1 was applied to 1-[2-({4-[4-(oxetan-3-yl)piperazin-1-yl]phenyl}amino)-5-[2-(triisopropylsilyl)ethynyl]pyrido[2,3-d]pyrimidin-7-yl]-1,3-diazaspiro[4.4]nonan-2-one (100 mg, 0.147 mmol) and potassium fluoride (85.3 mg, 1.47 mmol) in THF (2.0 mL), DMF (2.0 mL) and water (0.50 mL), stirred for 1 hour at 60° C. The residue was purified by reverse phase flash column chromatography eluting with acetonitrile 910-70%) in water (0.1% formic acid), to yield the title compound as an orange solid (12.0 mg, 15%).

(ES, m/z) [M+H]⁺=525.30

¹H-NMR (300 MHz, DMSO-d₆) δ 9.91 (s, 1H), 9.12 (s, 1H), 8.23 (s, 1H), 7.83 (s, 2H), 7.73 (s, 1H), 6.91 (d, J=8.6 Hz, 2H), 4.99 (s, 1H), 4.58 (t, J=6.6 Hz, 2H), 4.48 (t, J=5.7 Hz, 2H), 3.45 (s, 1H), 3.12 (s, 4H), 2.63 (s, 2H), 2.42 (s, 4H), 1.65 (s, 4H).

### Example 93

### N-Methyl-N-{4-[(7-{2-oxo-1,3-diazaspiro[4.4]nonan-1-yl}-5-[2-(triisopropylsilyl)ethynyl]pyrido[2,3-d] pyrimidin-2-yl)amino]phenyl}acetamide

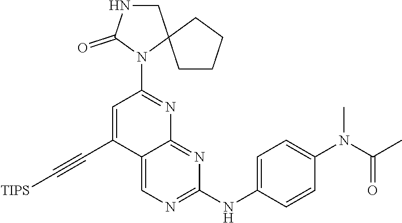

General Procedure 3 was applied to 1-{2-methanesulfinyl-5-[2-(triisopropylsilyl)ethynyl]pyrido[2,3-d]pyrimidin-7-yl}-1,3-diazaspiro[4.4]nonan-2-one (300 mg, 0.586 mmol), N-(4-aminophenyl)-N-methylacetamide (115 mg, 0.703 mmol) and trifluoroacetic acid (90.0 μL, 1.17 mmol) in 2-methyl-2-butanol (5 mL), stirred for 1 hour at 80° C. The residue was purified by reverse phase flash column chromatography eluting with acetonitrile (10-80%) in water (0.1% formic acid), to yield the title compound as a yellow solid (123 mg, 34%).

### N-{4-[(5-Ethynyl-7-{2-oxo-1,3-diazaspiro[4.4]nonan-1-yl}pyrido[2,3-d]pyrimidin-2-yl)amino]phenyl}-N-methylacetamide

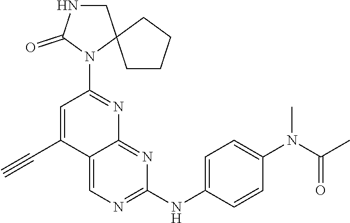 [SMILES: C#Cc1cc(N2C(=O)NCC23CCCC3)nc2nc(Nc3ccc(N(C)C(C)=O)cc3)ncc12]

General Procedure 1 was applied to N-methyl-N-{4-[(7-{2-oxo-1,3-diazaspiro[4.4]nonan-1-yl}-5-[2-(triisopropylsilyl)ethynyl]pyrido[2,3-d]pyrimidin-2-yl)amino]phenyl}acetamide (100 mg, 0.163 mmol) and potassium fluoride (94.9 mg, 1.63 mmol) in THF (2.0 mL), DMF (2.0 mL) and water (0.50 mL), stirred for 1 hour at 60° C. The residue was purified by reverse phase flash column chromatography eluting with acetonitrile (10-60%) in water (0.1% formic acid), to yield the title compound as a yellow solid (15.0 mg, 9%).

(ES, m/z) [M+H]⁺=456.15

¹H-NMR (300 MHz, DMSO-d₆) δ 10.27 (s, 1H), 9.20 (s, 1H), 8.30 (s, 1H), 8.06 (d, J=8.3 Hz, 2H), 7.77 (s, 1H), 7.27 (d, J=8.4 Hz, 2H), 5.02 (s, 1H), 3.14 (s, 3H), 2.63 (d, J=7.1 Hz, 2H), 2.26 (s, 2H), 1.79 (s, 3H), 1.66 (s, 4H).

### Example 94

### 2-(Dimethylamino)-N-methyl-N-{4-[(7-{2-oxo-1,3-diazaspiro[4.4]nonan-1-yl}-5-[2-(triisoprooylsilyl) ethynyl]pyrido[2,3-d]pyrimidin-2-yl)amino]phenyl}acetamide

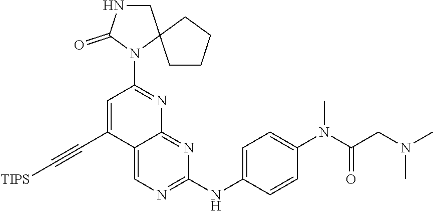

General Procedure 3 was applied to 1-{2-methanesulfinyl-5-[2-(triisopropylsilyl)ethynyl]pyrido[2,3-d]pyrimidin-7-yl}-1,3-diazaspiro[4.4]nonan-2-one (300 mg, 0.586 mmol), N-(4-aminophenyl)-2-(dimethylamino)-N-methylacetamide (146 mg, 0.703 mmol) and trifluoroacetic acid (90.0 μL, 1.17 mmol) in 2-methyl-2-butanol (5.0 mL), stirred for 1 hour at 80° C. The residue was purified by reverse phase flash column chromatography eluting with acetonitrile (10-70%) in water (0.1% formic acid), to yield the title compound as a yellow solid (112 mg, 29%).

### 2-(Dimethylamino)-N-{4-[(5-ethynyl-7-{2-oxo-1,3-diazaspiro[4.4]nonan-1-yl}pyrido[2,3-d]pyrimidin-2-yl)amino]phenyl}-N-methylacetamide

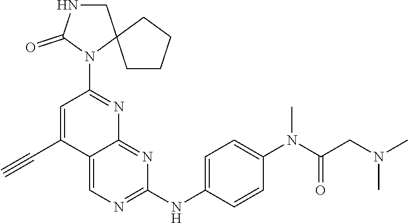 [SMILES: C#Cc1cc(N2C(=O)NCC23CCCC3)nc2nc(Nc3ccc(N(C)C(=O)CN(C)C)cc3)ncc12]

General Procedure 1 was applied to 2-(dimethylamino)-N-methyl-N-{4-[(7-{2-oxo-1,3-diazaspiro[4.4]nonan-1-yl}-5-[2-(triisopropylsilyl)ethynyl]pyrido[2,3-d]pyrimidin-2-yl)amino]phenyl}acetamide (100 mg, 0.153 mmol) and potassium fluoride (88.7 mg, 1.53 mmol) in THF (2.0 mL), DMF (2.0 mL) and water (0.50 mL), stirred for 1 hour at 60° C. The residue was purified by reverse phase flash column chromatography eluting with acetonitrile (10-60%) in water (0.1% formic acid), to yield the title compound as a yellow solid (33.4 mg, 44%).

(ES, m/z) [M+H]⁺=499.20

¹H-NMR (300 MHz, DMSO-d₆) δ 10.28 (s, 1H), 9.20 (s, 1H), 8.31 (s, 1H), 8.05 (d, J=8.4 Hz, 2H), 7.77 (s, 1H), 7.26 (d, J=8.4 Hz, 2H), 5.03 (s, 1H), 3.17 (d, J=17.8 Hz, 4H), 2.85 (s, 2H), 2.64 (s, 2H), 2.32-2.20 (m, 3H), 2.14 (s, 5H), 1.66 (s, 4H).

### Example 95

### 1-[2-({4-[2-(Dimethylamino)ethoxy]phenyl}amino)-5-[2-(triisoproylsilyl)ethynyl]pyrido[2,3-d]pyrimidin-7-yl]-1,3-diazaspiro[4.4]nonan-2-one

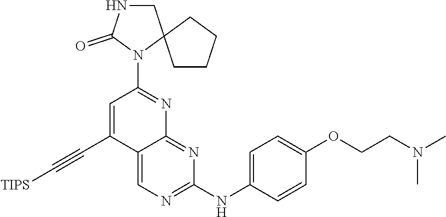

General Procedure 3 was applied to 1-{2-methanesulfinyl-5-[2-(triisopropylsilyl)ethynyl]pyrido[2,3-d]pyrimidin-7-yl}-1,3-diazaspiro[4.4]nonan-2-one (300 mg, 0.586 mmol), 4-[2-(dimethylamino)ethoxy]aniline (127 mg, 0.703 mmol) and trifluoroacetic acid (90.0 μL, 1.17 mmol) in 2-methyl-2-butanol (5.0 mL), stirred for 1 hour at 80° C. The residue was purified by reverse phase flash column chromatography eluting with acetonitrile (10-60%) in water (0.1% formic acid), to yield the title compound as a yellow solid (109 mg, 29%).

### 1-[2-({4-[2-(Dimethylamino)ethoxy]phenyl}amino)-5-ethynyl)pyrido[2,3-d]pyrimidin-7-yl]-1,3-diazaspiro[4.4]nonan-2-one

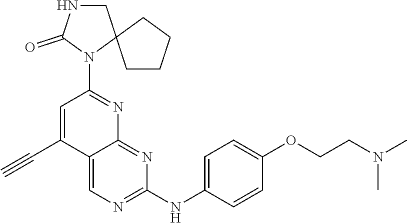 [SMILES: C#Cc1cc(N2C(=O)NCC23CCCC3)nc2nc(Nc3ccc(OCCN(C)C)cc3)ncc12]

General Procedure 1 was applied to 1-[2-({4-[2-(dimethylamino)ethoxy]phenyl}amino)-5-[2-(triisopropylsilyl)ethynyl]pyrido[2,3-d]pyrimidin-7-yl]-1,3-diazaspiro[4.4]nonan-2-one (100 mg, 0.159 mmol) and potassium fluoride (92.5 mg, 1.59 mmol) in THF (2.0 mL), DMF (2.0 mL) and water (0.50 mL), stirred for 1 hour at 60° C. The residue was purified by reverse phase flash column chromatography eluting with acetonitrile (10-50%) in water (0.1% formic acid), to yield the title compound as a yellow solid (23.5 mg, 31%).

(ES, m/z) [M+H]⁺=472.20

¹H-NMR (300 MHz, DMSO-d₆) δ 9.97 (s, 1H), 9.14 (s, 1H), 8.24 (s, 1H), 7.87 (s, 2H), 7.74 (s, 1H), 6.99-6.83 (m, 2H), 5.00 (s, 1H), 4.03 (t, J=5.8 Hz, 2H), 2.61 (t, J=5.8 Hz, 4H), 2.22 (s, 8H), 1.65 (s, 4H).

### Example 96

### 1-[2-({4-[2-(Dimethylamino)ethoxy]phenyl}amino)-5-[2-(triisopropylsilyl)ethynyl]pyrido[2,3-d]pyrimidin-7-yl]-1,3-diazaspiro[4.4]nonan-2-one

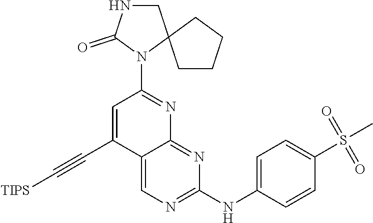

General Procedure 3 was applied to 1-{2-methanesulfinyl-5-[2-(triisopropylsilyl)ethynyl]pyrido[2,3-d]pyrimidin-7-yl}-1,3-diazaspiro[4.4]nonan-2-one (300 mg, 0.586 mmol), 4-[2-(dimethylamino)ethoxy]aniline (127 mg, 0.703 mmol) and trifluoroacetic acid (90.0 μL, 1.17 mmol) in 2-methyl-2-butanol (5.0 mL), stirred for 1 hour at 80° C. The residue was purified by reverse phase flash column chromatography eluting with acetonitrile (10-60%) in water (0.1% formic acid), to yield the title compound as a yellow solid (109 mg, 29%).

### 1-{5-Ethynyl-2-[(4-methanesulfonylphenyl)amino]pyrido[2,3-d]pyrimidin-7-yl}-1,3-diazaspiro[4.4]nonan-2-one

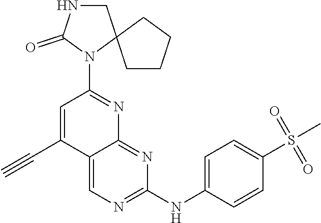 [SMILES: C#Cc1cc(N2C(=O)NCC23CCCC3)nc2nc(Nc3ccc(S(C)(=O)=O)cc3)ncc12]

General Procedure 1 was applied to 1-{2-[(4-methanesulfonylphenyl)amino]-5-[2-(triisopropylsilyl)ethynyl]pyrido[2,3-d]pyrimidin-7-yl}-1,3-diazaspiro[4.4]nonan-2-one (100 mg, 0.162 mmol) and potassium fluoride (93.9 mg, 1.62 mmol) in THF (2.0 mL), DMF (2.0 mL) and water (0.50 mL), stirred for 1 hour at 60° C. The residue was purified by reverse phase flash column chromatography eluting with acetonitrile (10-50%) in water (0.1% formic acid), to yield the title compound as a yellow solid (15.6 mg, 21%).

(ES, m/z) [M+H]⁺=463.05

¹H-NMR (300 MHz, DMSO-d₆) δ 10.66 (s, 1H), 9.27 (s, 1H), 8.38 (s, 1H), 8.31-8.23 (m, 2H), 7.93-7.74 (m, 3H), 5.06 (s, 1H), 3.19 (s, 3H), 2.75-2.57 (m, 3H), 2.29 (s, 3H), 1.69 (s, 4H).

### Example 97

### N,N-Dimethyl-4-[(7-{2-oxo-1,3-diazaspiro[4.4]nonan-1-yl}-5-[2-(triisopropylsilyl)ethynyl]pyrido[2,3-d]pyrimidin-2-yl)amino]benzenesulfonamide

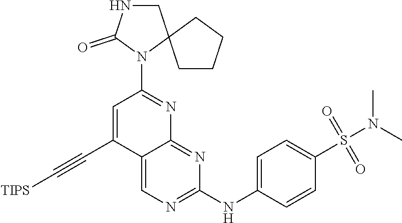

General Procedure 3 was applied to 1-{2-methanesulfinyl-5-[2-(triisopropylsilyl)ethynyl]pyrido[2,3-d]pyrimidin-7-yl}-1,3-diazaspiro[4.4]nonan-2-one (300 mg, 0.586 mmol), N¹-dimethylsulfanilamide (141 mg, 0.703 mmol) and trifluoroacetic acid (90.0 μL, 1.17 mmol) in 2-methyl-2-butanol (5.0 mL), stirred for 1 hour at 80° C. The residue was purified by reverse phase flash column chromatography eluting with acetonitrile (10-80%) in water (0.1% formic acid), to yield the title compound as a yellow solid (100 mg, 26%).

### 4-[(5-Ethynyl-7-{2-oxo-1,3-diazaspiro[4.4]nonan-1-yl}pyrido[2,3-d]pyrimidin-2-yl)amino]-N,N-dimethylbenzenesulfonamide

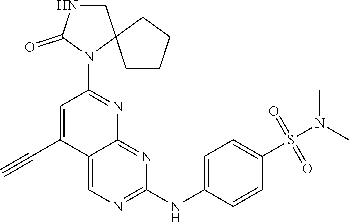 [SMILES: C#Cc1cc(N2C(=O)NCC23CCCC3)nc2nc(Nc3ccc(S(=O)(=O)N(C)C)cc3)ncc12]

General Procedure 1 was applied to N,N-dimethyl-4-[(7-{2-oxo-1,3-diazaspiro[4.4]nonan-1-yl}-5-[2-(triisopropylsilyl) ethynyl]pyrido[2,3-d]pyrimidin-2-yl)amino]benzenesulfonamide (100 mg, 0.154 mmol) and potassium fluoride (89. 7 mg, 1.54 mmol) in THE (2.0 mL), DMF (2.0 mL) and water (0.50 mL) was stirred for 1 hour at 60° C. The residue was purified by reverse phase flash column chromatography eluting with acetonitrile (10-50%) in water (0.1% formic acid), to yield the title compound as a yellow solid (33.0 mg, 43%).

(ES, m/z) [M+H]⁺=492.00

¹H-NMR (300 MHz, DMSO-d₆) δ 10.63 (s, 1H), 9.26 (s, 1H), 8.37 (s, 1H), 8.26 (d, J=8.8 Hz, 2H), 7.80 (s, 1H), 7.71-7.64 (m, 2H), 5.05 (s, 1H), 2.65 (s, 2H), 2.60 (s, 6H), 2.28 (s, 2H), 1.68 (s, 4H).

### Example 98

### N-[2-(Dimethylamino)ethyl]-N-methyl-4-[(7-{2-oxo-1,3-diazaspiro[4.4]nonan-1-yl}-5-[2-(triisopropylsilyl)ethynyl]pyrido[2,3-d]pyrimidin-2-yl)amino]benzenesulfonamide

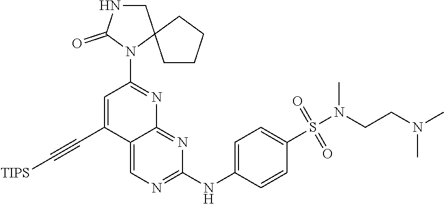

General Procedure 3 was applied to 1-{2-methanesulfinyl-5-[2-(triisopropylsilyl)ethynyl]pyrido[2,3-d]pyrimidin-7-yl}-1,3-diazaspiro[4.4]nonan-2-one (200 mg, 0.391 mmol), 4-amino-N-[2-(dimethylamino)ethyl]-N-methylbenzenesulfonamide (121 mg, 0.469 mmol) and trifluoroacetic acid (60.0 μL, 0.782 mmol) in 2-methyl-2-butanol (2.0 mL), stirred overnight at 110° C. The residue was purified by reverse phase flash column chromatography eluting with acetonitrile (10-80%) in water (0.1% formic acid), to yield the title compound as a yellow solid (134 mg, 48%).

### N-[2-(dimethylamino)ethyl]-4-[(5-ethynyl-7-{2-oxo-1,3-diazaspiro[4.4]nonan-1-yl}pyrido[2,3-d]pyrimidin-2-yl)amino]-N-methylbenzenesulfonamide

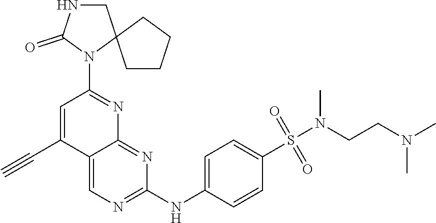 [SMILES: C#Cc1cc(N2C(=O)NCC23CCCC3)nc2nc(Nc3ccc(S(=O)(=O)N(C)CCN(C)C)cc3)ncc12]

General Procedure 1 was applied to N-[2-(dimethylamino)ethyl]-N-methyl-4-[(7-{2-oxo-1,3-diazaspiro[4.4]nonan-1-yl}-5-[2-(triisopropylsilyl)ethynyl]pyrido[2,3-d]pyrimidin-2-yl)amino]benzenesulfonamide (75.0 mg, 0.106 mmol) and potassium fluoride (30.9 mg, 0.530 mmol) in DMF (2.0 mL), stirred for 1 hour at room temperature. The resulting mixture was diluted with water (10 mL). The precipitated solids were collected by filtration washing with water (2×5 mL) to yield the title compound as a light yellow solid (5.00 mg, 8%).

(ES, m/z) [M+H]⁺=549.20

¹H-NMR (400 MHz, DMSO-d₆) δ 10.60 (s, 1H), 9.25 (s, 1H), 8.36 (s, 1H), 8.23 (d, J=8.6 Hz, 2H), 7.80 (s, 1H), 7.74-7.69 (m, 2H), 5.04 (s, 1H), 3.02 (t, J=6.7 Hz, 2H), 2.68 (s, 3H), 2.65 (d, J=8.8 Hz, 2H), 2.37 (t, J=6.7 Hz, 2H), 2.28 (s, 2H), 2.14 (s, 6H), 1.67 (s, 4H).

### Example 99

### (5S)-5-Isopropyl-1-(2-{[4-(4-methylpiperazin-1-yl)phenyl]amino}-5-[2-(triisopropylsilyl)ethynyl]pyrido[2,3-d]pyrimidin-7-yl)imidazolidin-2-one

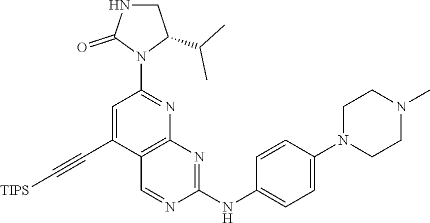

The crude product (250 mg) was purified by Prep-HPLC with the following conditions (Mobile Phase: (MtBE:Hex=1:1)(0.1% Dethyl acetate):methanol=90:10, Flow rate: 1.67 ml/min, Temperature:Ambient) to afford the title compound (120 mg, 48%) as a yellow solid.

### (5S)-1-(5-Ethynyl-2-{[4-(4-methylpiperazin-1-yl)phenyl]amino}pyrido[2,3-d]pyrimidin-7-yl)-5-isopropylmidazolidin-2-one

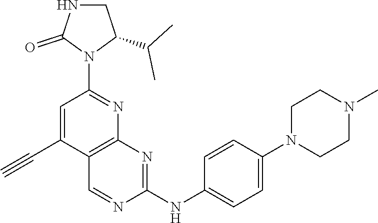 [SMILES: C#Cc1cc(N2C(=O)NC[C@@H]2C(C)C)nc2nc(Nc3ccc(N4CCN(C)CC4)cc3)ncc12]

General Procedure 1 was applied to (5S)-5-isopropyl-1-(2-{[4-(4-methylpiperazin-1-yl)phenyl]amino}-5-[2-(triisopropylsilyl)ethynyl]pyrido[2,3-d]pyrimidin-7-yl)imidazolidin-2-one (120 mg, 0.191 mmol) and potassium fluoride (111 mg, 1.91 mmol) in THF (2.0 mL), DMF (2.0 mL) and water (0.5 mL), stirred for 1 hour at 60° C. The residue was purified by reverse phase flash column chromatography eluting with acetonitrile (10-50%) in water (0.1% formic acid), to yield the title compound as an orange solid (26.0 mg, 29%).

(ES, m/z) [M+H]⁺=471.05

¹H-NMR (300 MHz, DMSO-d₆) δ 9.90 (s, 1H), 9.11 (s, 1H), 8.40 (s, 1H), 7.67 (s, 3H), 6.99-6.84 (m, 2H), 5.00 (s, 1H), 4.79 (d, J=8.8 Hz, 1H), 3.47 (t, J=9.5 Hz, 1H), 3.29 (s, 1H), 3.09 (t, J=4.9 Hz, 4H), 2.68-2.58 (m, 1H), 2.46 (t, J=5.0 Hz, 4H), 2.23 (s, 3H), 0.93 (d, J=7.0 Hz, 3H), 0.77 (d, J=6.9 Hz, 3H).

### Example 100

### (5S)-5-Isopropyl-1-(2-{[4-(4-methylpiperazin-1-yl)phenyl]amino}-5-[2-(triisopropylsilyl)ethynyl]pyrido[2,3-d]pyrimidin-7-yl)imidazolidin-2-one

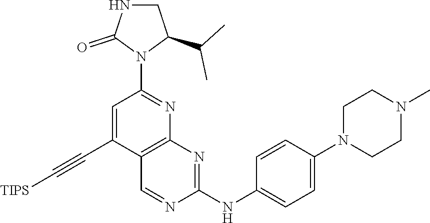

The crude product (250 mg) was purified by Prep-HPLC with the following conditions (Mobile Phase:(MtBE:Hex=1:1)(0.1% Dethyl acetate): methanol=90:10, Flow rate: 1.67 ml/min, Temperature: Ambient) to afford the title compound (120 mg, 48%) as a yellow solid.

### (5R)-1-(5-Ethynyl-2-{[4-(4-methylpiperazin-1-yl)phenyl]amino}pyrido[2,3-d]pyrimidin-7-yl)-5-isopropylmidazolidin-2-one

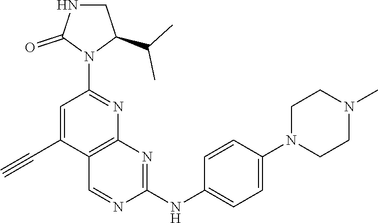 [SMILES: C#Cc1cc(N2C(=O)NC[C@H]2C(C)C)nc2nc(Nc3ccc(N4CCN(C)CC4)cc3)ncc12]

General Procedure 1 was applied to (5R)-5-isopropyl-1-(2-{[4-(4-methylpiperazin-1-yl)phenyl]amino}-5-[2-(triisopropylsilyl)ethynyl]pyrido[2,3-d]pyrimidin-7-yl)imidazolidin-2-one (120 mg, 0.191 mmol) and potassium fluoride (111 mg, 1.91 mmol) in THF (2.0 mL), DMF (2.0 mL) and water (2.0 mL), stirred for 1 hour at 60° C. The residue was purified by reverse phase flash column chromatography eluting with acetonitrile (10-60%) in water (0.1% formic acid), to yield the title compound as an orange solid (22.0 mg, 24%).

(ES, m/z): [M+H]⁺=471.05

¹H-NMR (300 MHz, DMSO-d₆) δ 9.90 (s, 1H), 9.11 (s, 1H), 8.40 (s, 1H), 7.67 (s, 3H), 6.96-6.86 (m, 2H), 5.00 (s, 1H), 4.79 (d, J=9.3 Hz, 1H), 3.47 (t, J=9.5 Hz, 1H), 3.25 (s, 1H), 2.70-2.59 (m, 1H), 2.46 (t, J=5.0 Hz, 4H), 2.23 (s, 3H), 0.93 (d, J=6.9 Hz, 3H), 0.77 (d, J=6.8 Hz, 3H).

### Example 101

### tert-Butyl N-[(2S)-2-{[(benzyloxy)carbonyl]amino}-3-hydroxypropyl]carbamate

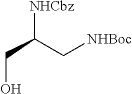 [SMILES: CC(C)(C)OC(=O)NC[C@@H](CO)NC(=O)OCc1ccccc1]

A solution of (2S)-2-{[(benzyloxy)carbonyl]amino}-3-[(tert-butoxycarbonyl)amino]propanoic acid (10.0 g, 29.5 mmol, 1.0 eq.), NMM (3.29 g, 32.5 mmol, 1.1 eq.) and propan-2-yl carbonochloridate (3.98 g, 32.5 mmol, 1.1 eq.) in THF (100 mL) was stirred for 1 hour at 0° C. The resulting mixture was filtered, washing with THF (20 mL). The filtrate was concentrated under reduced pressure, then sodium borohydride (1.23 g, 32.5 mmol, 1.1 eq.) added in portions over 2 minutes at 0° C. The resulting mixture was stirred for an additional 2 hours at 0° C. then filtered, washing with ethyl acetate (2×50 mL). The filtrate was concentrated under reduced pressure. The resulting residue was diluted with water (20 mL) and extracted with ethyl acetate (2×100 mL). The combined organic layers were concentrated under reduced pressure. The residue was purified by reverse phase flash column chromatography eluting with acetonitrile (10-80%) in water (0.1% formic acid), to yield the title compound as a white solid (5.70 g, 59%).

### Benzyl N-[(2S)-1-[(tert-butoxycarbonylAamino]-3-methoxypropan-2-yl]carbamate

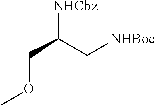 [SMILES: COC[C@H](CNC(=O)OC(C)(C)C)NC(=O)OCc1ccccc1]

A solution of tert-butyl N-[(2S)-2-{[(benzyloxy)carbonyl]amino}-3-hydroxypropyl]carbamate (3.70 g, 11.4 mmol, 1.0 eq.), N¹,N¹,N⁸,N³-tetramethylnaphthalene-1,8-diamine (6.11 g, 28.5 mmol, 2.5 eq.) and tetrafluoroboranuide trimethyloxidanium (4.22 g, 28.5 mmol, 2.5 eq.) in dichloromethane (30 mL) was stirred overnight at room temperature. The resulting mixture was concentrated under reduced pressure. The residue was purified by reverse phase flash column chromatography eluting with acetonitrile (10-60%) in water (0.1% formic acid), to yield the title compound as a white solid (1.40 g, 36%).

### tert-Butyl N-[(2S)-2-amino-3-methoxypropyl]carbamate

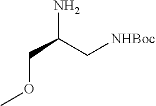

A solution of benzyl N-[(2S)-1-[(tert-butoxycarbonyl)amino]-3-methoxypropan-2-yl]carbamate (1.20 g, 3.54 mmol, 1.0 eq.) and palladium on carbon (300 mg, 2.84 mmol, 0.8 eq.) in dichloromethane (20 mL) was stirred for overnight at room temperature. The resulting mixture was concentrated under reduced pressure to yield the title compound as a white solid (600 mg, 83%).

### tert-Butyl N-[(2S)-3-methoxy-2-{[2-(methylsulfanyl)-5-[2-(triisopropylsilyl)ethynyl]pyrido[2,3-d]pyrimidin-7-yl]amino}propyl]carbamate

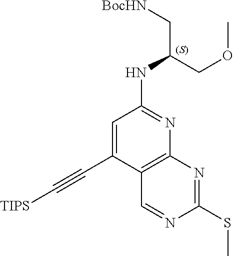

A solution of 7-chloro-2-(methylsulfanyl)-5-[2-(triisopropylsilyl)ethynyl]pyrido[2,3-d]pyrimidine (800 mg, 2.04 mmol, 1.0 eq.), tert-butyl N-[(2S)-2-amino-3-methoxypropyl]carbamate (542 mg, 2.65 mmol, 1.3 eq.), potassium carbonate (564 mg, 4.08 mmol, 2.0 eq.), tris(dibenzyldeneacetone)dipalladium (0) (93.4 mg, 0.102 mmol, 0.05 eq.) and XantPhos (118 mg, 0.204 mmol, 0.10 eq.) in dioxane (8.0 mL) was stirred for 1 hour at 100° C. The mixture was allowed to cool down to room temperature. The resulting mixture was concentrated under reduced pressure. The residue was purified by flash column chromatography eluting with ethyl acetate (25%) in 40-60 petroleum ether to afford the title compound as a yellow solid (524 mg, 46%).

### tert-Butyl N-[(2S)-2-({2-methanesulfonyl-5-[2-(triisopropylsilyl)ethynyl]pyrido[2,3-d]pyrimidin-7-yl}amino)-3-methoxypropyl]carbamate

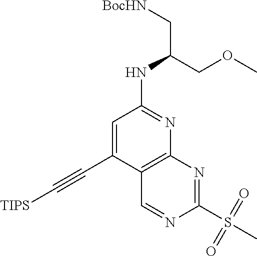

General Procedure 2 was applied to tert-butyl N-[(2S)-3-methoxy-2-{[2-(methylsulfanyl)-5-[2-(triisopropylsilyl)ethynyl]pyrido[2,3-d]pyrimidin-7-yl]amino}propyl]carbamate (450 mg, 0.804 mmol, 1.0 eq.) and m-CPBA (139 mg, 0.804 mmol) in dichloromethane (8.0 mL). The residue was purified by flash column chromatography eluting with ethyl acetate (10%) in 40-60 petroleum ether to afford the title compound as a yellow solid (450 mg, 94%).

### tert-Butyl N-[(2S)-3-methoxy-2-[(2-{[4-(4-methylpiperazin-1-yl)phenyl]amino}-5-[2-(triisopropylsilyl)ethynyl]pyrido[2,3-d]pyrimidin-7-yl)amino]propyl]carbamate

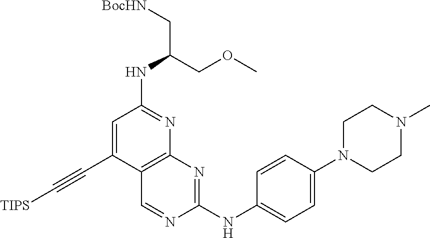

General Procedure 3 was applied to tert-butyl N-[(2S)-2-({2-methanesulfonyl-5-[2-(triisopropylsilyl)ethynyl]pyrido[2,3-d]pyrimidin-7-yl}amino)-3-methoxypropyl]carbamate (420 mg, 0.710 mmol), 4-(4-methylpiperazin-1-yl)aniline (176 mg, 0.923 mmol) and trifluoroacetic acid (0.110 mL, 1.42 mmol) in 2-methyl-2-butanol (8.0 mL) was stirred for 1 hour at 80° C. The residue was purified by reverse phase flash column chromatography eluting with acetonitrile (10-50%) in water (0.1% formic acid), to yield the title compound as a yellow solid (292 mg, 58%).

### N⁷-[(2S)-1-Amino-3-methoxypropan-2-yl]-N²-[4-(4-methylpiperazin-1-yl)phenyl]-5-[2-(triisopropylsilyl)ethynyl]pyrido[2,3-d]pyrimidine-2,7-diamine

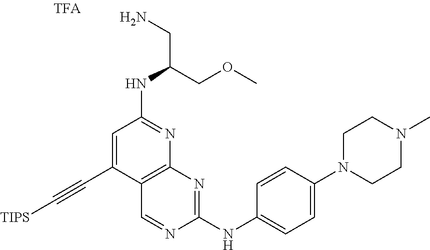

A solution of tert-butyl N-[(2S)-3-methoxy-2-[(2-{[4-(4-methylpiperazin-1-yl)phenyl]amino}-5-[2-(triisopropylsilyl)ethynyl]pyrido[2,3-d]pyrimidin-7-yl)amino]propyl]carbamate (330 mg, 0.469 mmol, 1.0 eq.) and trifluoroacetic acid (0.170 mL, 2.34 mmol, 5.0 eq.) in dichloromethane (5.0 mL) was stirred for 1 hour at room temperature. The resulting mixture was concentrated under reduced pressure to yield the title compound as a yellow solid (210 mg, 74%).

### (5S)-5-(Methoxymethyl)-1-(2-{[4-(4-methylpiperazin-1-yl)phenyl]amino}-5-[2-(triisopropylsilyl)ethynyl]pyrido[2,3-d]pyrimidin-7-yl)imidazolidin-2-one

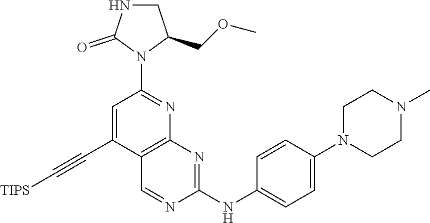

A solution of N⁷-[(2S)-1-amino-3-methoxypropan-2-yl]-N²-[4-(4-methylpiperazin-1-yl)phenyl]-5-[2-(triisopropylsilyl)ethynyl]pyrido[2,3-d]pyrimidine-2,7-diamine (300 mg, 0.498 mmol, 1.0 eq.), CDI (121.03 mg, 0.747 mmol, 1.5 eq.) and diisopropylethylamine (0.220 mL, 1.25 mmol, 2.5 eq.) in DMF (2.0 mL) was stirred for2 hours at 80° C. The mixture was allowed to cool down to room temperature. The residue was purified by reverse phase flash column chromatography eluting with acetonitrile (10-80%) in water (0.1% formic acid), to yield the title compound as a yellow solid (220 mg, 70%).

### (5S)-1-(5-Ethynyl-2-{[4-(4-methylpiperazin-1-yl)phenyl]amino}pyrido[2,3-d]pyrimidin-7-yl)-5-(methoxymethyl)imidazolidin-2-one

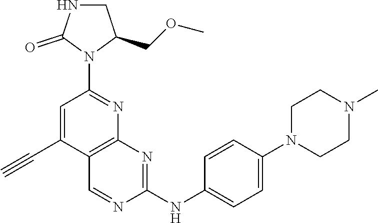 [SMILES: C#Cc1cc(N2C(=O)NC[C@H]2COC)nc2nc(Nc3ccc(N4CCN(C)CC4)cc3)ncc12]

General Procedure 1 was applied to (5S)-5-(methoxymethyl)-1-(2-{[4-(4-methylpiperazin-1-yl)phenyl]amino}-5-[2-(triisopropylsilyl)ethynyl]pyrido[2,3-d]pyrimidin-7-yl)imidazolidin-2-one (160 mg, 0.254 mmol) and potassium fluoride (148 mg, 2.54 mmol) in THF (2.0 mL), DMF (2.0 mL) and water (0.50 mL), stirred for 1 hour at 60° C. The residue was purified by reverse phase flash column chromatography eluting with acetonitrile (10-60%) in water (0.1% formic acid), to yield the title compound as an orange solid (30.0 mg, 25%).

(ES, m/z) [M+H]⁺=473.05

¹H-NMR (300 MHz, DMSO-d₆) δ 9.91 (s, 1H), 9.12 (s, 1H), 8.37 (s, 1H), 7.71 (s, 3H), 7.00-6.86 (m, 2H), 5.01 (s, 1H), 4.93 (s, 1H), 3.73-3.54 (m, 3H), 3.10 (t, J=5.0 Hz, 4H), 2.46 (t, J=5.0 Hz, 4H), 2.23 (s, 3H).

### Example 102

### 4-Isopropyl-1-methyl-3-[2-(methylsulfanyl)-5-[2-(triisopropylsilyl)ethynyl]pyrido[2,3-d]pyrimidin-7-yl]imidazolidin-2-one

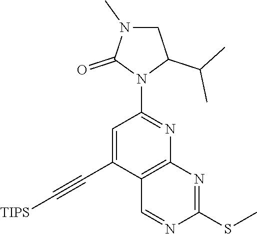

A solution of 5-isopropyl-1-[2-(methylsulfanyl)-5-[2-(triisopropylsilyl)ethynyl]pyrido[2,3-d]pyrimidin-7-yl]imidazolidin-2-one (1.50 g, 3.10 mmol, 1.0 eq.), methyl iodide (660 mg, 4.65 mmol, 1.5 eq.) and potassium carbonate (860 mg, 6.20 mmol, 2.0 eq.) in DMF (20 mL) was stirred overnight at room temperature. The resulting mixture was diluted with water (100 mL). The precipitated solids were collected by filtration, washing with water (2×10 mL) to yield the title compound as a yellow solid (1.45 g, 94%).

### 4-Isopropyl-3-{2-methanesuifonyl-5-[2-(triisopropylsilyl)ethynyl]pyrido[2,3-d]pyrimidin-7-yl}-1-methylmidazolidin-2-one

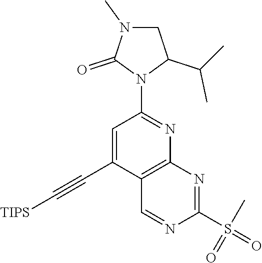

General Procedure 2 was applied to 4-isopropyl-1-methyl-3-[2-(methylsulfanyl)-5-[2-(triisopropylsilyl)ethynyl]pyrido[2,3-d]pyrimidin-7-yl]imidazolidin-2-one (1.45 g, 2.91 mmol) and m-CPBA (1.11 g, 6.41 mmol) in dichloromethane (20 mL) to yield the title compound as a yellow solid (1.34 g, 87%).

### 4-Isopropyl-1-methyl-3-(2-{[4-(4-methylpiperazin-1-yl)phenyl]amino}-5-[2-(triisopropylsilyl)ethynyl]pyrido[2,3-d]pyrimidin-7-yl)imidazolidin-2-one

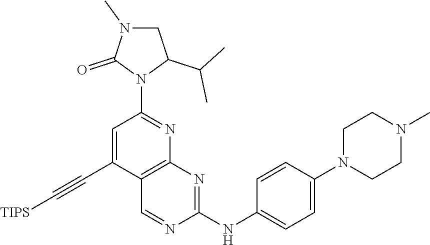

General Procedure 3 was applied to 4-isopropyl-3-{2-methanesulfonyl-5-[2-(triisopropylsilyl)ethynyl]pyrido[2,3-d]pyrimidin-7-yl}-1-methylmidazolidin-2-one (500 mg, 0.944 mmol), 4-(4-methylpiperazin-1-yl)aniline (216 mg, 1.13 mmol) and trifluoroacetic acid (0.140 mL, 1.89 mmol) in 2-methyl-2-butanol (5.0 mL), stirred for 1 hour at 100° C. The residue was purified by reverse phase flash column chromatography eluting with acetonitrile (10-60%) in water (0.1% formic acid), to yield the title compound as a yellow solid (134 mg, 22%).

### 3-(5-Ethynyl-2-{[4-(4-methylpiperazin-1-yl)phenyl]amino}pyrido[2,3-d]pyrimidin-7-yl)-4-isopropyl-1-methylmidazolidin-2-one

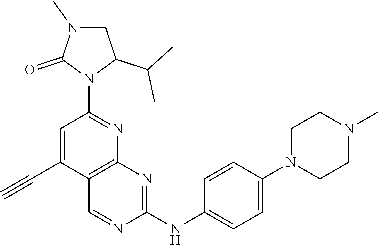 [SMILES: C#Cc1cc(N2C(=O)N(C)CC2C(C)C)nc2nc(Nc3ccc(N4CCN(C)CC4)cc3)ncc12]

General Procedure 1 was applied to 4-isopropyl-1-methyl-3-(2-{[4-(4-methylpiperazin-1-yl)phenyl]amino}-5-[2-(triisopropylsilyl)ethynyl]pyrido[2,3-d]pyrimidin-7-yl)imidazolidin-2-one (86.0 mg, 0.134 mmol) and potassium fluoride (77.9 mg, 1.34 mmol) in THF (2.0 mL), DMF (2.0 mL) and water (0.50 mL), stirred for 1 hour at 60° C. The residue was purified by reverse phase flash column chromatography eluting with acetonitrile (10-60%) in water (0.1% formic acid), to yield the title compound as an orange solid (16.0 mg, 24%).

(ES, m/z): [M+H]⁺=485.10

¹H-NMR (300 MHz, DMSO-d₆) δ 9.99 (d, J=8.5 Hz, 1H), 9.13 (s, 1H), 8.42 (s, 1H), 7.77 (s, 3H), 7.02 (d, J=9.0 Hz, 2H), 5.02 (s, 1H), 4.79 (d, J=8.8 Hz, 1H), 3.58 (t, J=5.1 Hz, 4H), 3.48 (d, J=5.5 Hz, 4H), 3.27 (d, J=6.5 Hz, 1H), 3.20 (s, 6H), 2.68-2.58 (m, 1H), 0.93 (d, J=7.0 Hz, 3H), 0.78 (d, J=6.9 Hz, 3H).

### Example 103

### 3-Methyl-1-[2-(methylsulfanyl)-5-[2-(triisopropylsilyl)ethynyl]pyrido[2,3-d]pyrimidin-7-yl]-1,3-diazaspiro[4.4]nonan-2-one

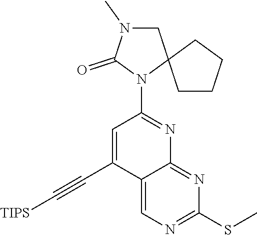

A solution of 1-[2-(methylsulfanyl)-5-[2-(triisopropylsilyl)ethynyl]pyrido[2,3-d]pyrimidin-7-yl]-1,3-diazaspiro[4.4]nonan-2-one (2.00 g, 4.03 mmol, 1.0 eq.), methyl iodide (630 mg, 4.44 mmol, 1.1 eq.) and sodium hydride (190 mg, 8.07 mmol, 2.0 eq.) in THF (10 mL) was stirred for 2 hours at 0° C. The reaction was quenched with saturated aqueous ammonium chloride solution and extracted with ethyl acetate (3×15 mL). The combined organic layers were dried (Na₂SO₄) and concentrated under reduced pressure to yield the title compound as a light yellow solid (1.07 g, 52%).

### 1-{2-Methanesulfinyl-5-[2-(triisopropylsilyl)ethynyl]pyrido[2,3-d]pyrimidin-7-yl}-3-methyl-1,3-diazaspiro[4.4]nonan-2-one

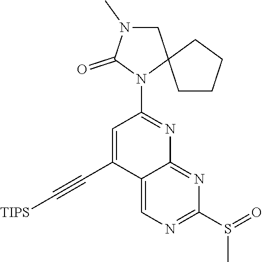

General Procedure 2 was applied to 3-methyl-1-[2-(methylsulfanyl)-5-[2-(triisopropylsilyl)ethynyl]pyrido[2,3-d]pyrimidin-7-yl]-1,3-diazaspiro[4.4]nonan-2-one (1.07 g, 2.10 mmol) and m-CPBA (360 mg, 2.10 mmol) in dichloromethane (10 mL). The residue was purified by flash column chromatography eluting with ethyl acetate (10%) in 40-60 petroleum ether to afford the title compound as a light yellow solid (865 mg, 78%).

### N-Methyl-N-{4-[(7-{3-methyl-2-oxo-1,3-diazaspiro[4.4]nonan-1-yl}-5-[2-(triisopropylsilyl)ethynyl]pyrido[2,3-d]pyrimidin-2-yl)amino]phenyl}acetamide

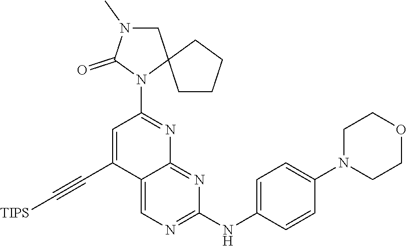

General Procedure 3 was applied to 1-{2-methanesulfinyl-5-[2-(triisopropylsilyl)ethynyl]pyrido[2,3-d]pyrimidin-7-yl}-3-methyl-1,3-diazaspiro[4.4]nonan-2-one (200 mg, 0.380 mmol), 4-(morpholin-4-yl)aniline (81.3 mg, 0.456 mmol) and trifluoroacetic acid (86.7 mg, 0.760 mmol) in 2-butanol (5.0 mL), stirred for 2 hours at 80° C. to yield the title compound as a yellow solid (191 mg, 80%) The crude product mixture was used in the next step directly without further purification.

### 1-(5-Ethynyl-2-{[4-(morpholin-4-yl)phenyl]amino}pyrido[2,3-d]pyrimidin-7-yl)-3-methyl-1,3-diazaspiro[4.4]nonan-2-one

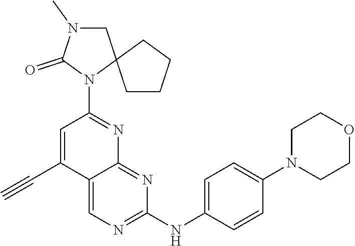 [SMILES: C#Cc1cc(N2C(=O)N(C)CC23CCCC3)nc2nc(Nc3ccc(N4CCOCC4)cc3)ncc12]

General Procedure 1 was applied to 3-methyl-1-(2-{[4-(morpholin-4-yl)phenyl]amino}-5-[2-(triisopropylsilyl)ethynyl]pyrido [2,3-d]pyrimidin-7-yl)-1,3-diazaspiro[4.4]nonan-2-one (150 mg, 0.234 mmol) and potassium fluoride (136 mg, 2.34 mmol) in THF (0.50 mL), DMF (0.50 mL) and water (0.10 mL), stirred for 1 hour at 60° C. The residue was purified by reverse phase flash column chromatography eluting with acetonitrile (10-50%) in water (0.1% formic acid), to yield the title compound as a brown solid (11.0 mg, 9%).

(ES, m/z) [M+H]⁺=484.15

¹H-NMR (400 MHz, DMSO-d₆) δ 9.91 (s, 1H), 9.12 (s, 1H), 8.27 (s, 1H), 7.85 (s, 2H), 6.91 (d, J=8.9 Hz, 2H), 4.98 (s, 1H), 3.74 (t, J=4.8 Hz, 3H), 3.37 (s, 2H), 3.06 (t, J=4.8 Hz, 3H), 2.85 (s, 3H), 2.64 (s, 2H), 2.26 (s, 2H), 1.64 (d, J=11.4 Hz, 4H).

### Example 104

### 3-Methyl-1-[2-({4-[4-(oxetan-3-yl)piperazin-1-yl]phenyl}amino)-5-[2-(triisopropylsilyl)ethynyl]pyrido[2,3-d]pyrimidin-7-yl]-1,3-diazaspiro[4.4]nonan-2-one

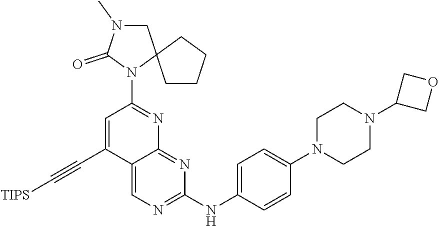

General Procedure 3 was applied to 1-{2-methanesulfinyl-5-[2-(triisopropylsilyl)ethynyl]pyrido[2,3-d]pyrimidin-7-yl}-3-methyl-1,3-diazaspiro[4.4]nonan-2-one (200 mg, 0.380 mmol), 4-[4-(oxetan-3-yl)piperazin-1-yl]aniline (106 mg, 0.456 mmol) and trifluoroacetic acid (60.0 μL, 0.760 mmol) in 2-methyl-2-butanol (2.0 mL), stirred for 1 hour at 80° C. to yield the title compound as a light yellow solid (103 mg, 39%).

### 1-[5-Ethynyl-2-([4-[4-(oxetan-3-yl)piperazin-1-yl]phenyl]amino)pyrido[2,3-d]pyrimidin-7-yl]-3-methyl-1,3-diazaspiro[4.4]nonan-2-one

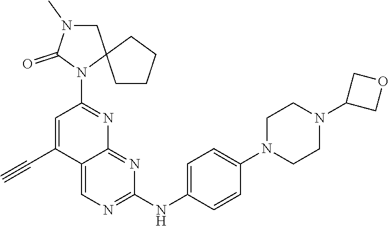 [SMILES: C#Cc1cc(N2C(=O)N(C)CC23CCCC3)nc2nc(Nc3ccc(N4CCN(C5COC5)CC4)cc3)ncc12]

General Procedure 1 was applied to 3-methyl-1-[2-({4-[4-(oxetan-3-yl)piperazin-1-yl]phenyl}amino)-5-[2-(triisopropylsilyl)ethynyl]pyrido[2,3-d]pyrimidin-7-yl]-1,3-diazaspiro[4.4]nonan-2-one (100 mg, 0.144 mmol) and potassium fluoride (41.8 mg, 0.720 mmol) in DMF (2.0 mL) was stirred for 1 hour at 60° C. The residue was purified by reverse phase flash column chromatography eluting with acetonitrile (10-50%) in water (0.1% formic acid), to yield the title compound as an orange solid (51.0 mg, 66%).

(ES, m/z) [M+H]⁺=539.30

¹H-NMR (300 MHz, DMSO-d₆) δ 9.91 (s, 1H), 9.12 (s, 1H), 8.27 (s, 1H), 7.83 (s, 2H), 6.91 (d, J=9.1 Hz, 2H), 4.99 (s, 1H), 4.58 (t, J=6.5 Hz, 2H), 4.47 (t, J=6.0 Hz, 2H), 3.46 (q, J=6.2 Hz, 1H), 3.11 (t, J=4.9 Hz, 4H), 2.85 (s, 3H), 2.64 (s, 2H), 2.42 (d, J=5.4 Hz, 4H), 2.27 (s, 2H), 1.65 (s, 4H).

### Example 105

### N-Methyl-N-{4-[(7-{3-methyl-2-oxo-1,3-diazaspiro[4.4]nonan-1-yl}-5-[2-(triisopropylsilyl)ethynyl]pyrido[2,3-d]pyrimidin-2-yl)amino]phenyl}acetamide

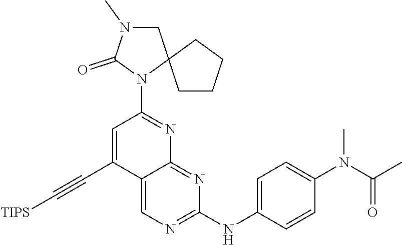

General Procedure 3 was applied to 1-{2-methanesulfinyl-5-[2-(triisopropylsilyl)ethynyl]pyrido[2,3-d]pyrimidin-7-yl}-3-methyl-1,3-diazaspiro[4.4]nonan-2-one (200 mg, 0.380 mmol), N-(4-aminophenyl)-N-methylacetamide (74.9 mg, 0.456 mmol) and trifluoroacetic acid (86.7 mg, 0.760 mmol) in 2-butanol (6.0 mL), stirred for 2 hours at 80 0C. The resulting mixture was diluted with (Na₂SO₄) and concentrated under reduced pressure to yield the title compound as a yellow solid (191 mg, 80%).

### N-{4-[(5-Ethynyl-7-{3-methyl-2-oxo-1,3-diazaspiro[4.4]nonan-1-yl}pyrido[2,3-d]pyrimidin-2-yl)amino]phenyl}-N-methylacetamide

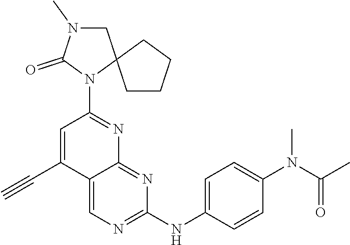 [SMILES: C#Cc1cc(N2C(=O)N(C)CC23CCCC3)nc2nc(Nc3ccc(N(C)C(C)=O)cc3)ncc12]

General Procedure 1 was applied to N-methyl-N-{4-[(7-{3-methyl-2-oxo-1,3-diazaspiro[4.4]nonan-1-yl}-5-[2-(triisopropylsilyl)ethynyl]pyrido[2,3-d]pyrimidin-2-yl)amino]phenyl}acetamide (150 mg, 0.240 mmol) and potassium fluoride (139 mg, 2.40 mmol) in THF (0.50 mL), DMF (0.50 mL) and water (0.10 mL), stirred for 1 hour at 60° C. The residue was purified by reverse phase flash column chromatography eluting with acetonitrile (10-50%) in water (0.1% formic acid), to yield the title compound as a yellow solid (32.0 mg, 28%).

(ES, m/z) [M+H]⁺=470.10

¹H-NMR (400 MHz, DMSO-d₆) δ 10.26 (s, 1H), 9.19 (s, 1H), 8.34 (s, 1H), 8.06 (d, J=8.3 Hz, 2H), 7.27 (d, J=8.4 Hz, 2H), 5.02 (s, 1H), 3.37 (s, 2H), 3.14 (s, 3H), 2.85 (s, 3H), 2.64 (dd, J=13.0, 5.5 Hz, 2H), 2.27 (s, 2H), 1.78 (s, 3H), 1.65 (dq, J=14.8, 9.2 Hz, 4H).

### Example 106

### 2-(Dimethylamino)-N-methyl-N-{4-[(7-{3-methyl-2-oxo-1,3-diazaspiro[4.4]nonan-1-yl}-5-[2 (triisopropylsilyl)ethynyl]pyrido[2,3-d]pyrimidin-2-yl)amino}phenyl]acetamide

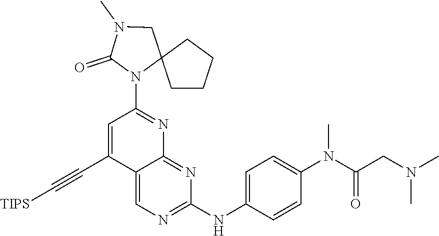

General Procedure 3 was applied to 1-{2-methanesulfinyl-5-[2-(triisopropylsilyl)ethynyl]pyrido[2,3-d]pyrimidin-7-yl}-3-methyl-1,3-diazaspiro[4.4]nonan-2-one (200 mg, 0.380 mmol), N-(4-aminophenyl)-2-(dimethylamino)-N-methylacetamide (94.6 mg, 0.456 mmol) and trifluoroacetic acid (86.7 mg, 0.760 mmol) in 2-butanol (6.0 mL) was stirred for 2 hours at 80° C. The resulting mixture was diluted with water (10 mL) and extracted with ethyl acetate (3×10 mL). The combined organic layers were dried (Na₂SO₄) and concentrated under reduced pressure to yield the title compound as a yellow solid (186 mg, 73%).

### 2-(Dimethylamino)-N-{4-[(5-ethynyl-7-{3-methyl-2-oxo-1,3-diazaspiro[4.4]nonan-1-yl}pyrido[2,3-d]pyrimidin-2-yl)amino}phenyl]-N-methylacetamide

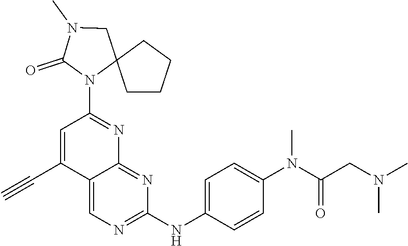 [SMILES: C#Cc1cc(N2C(=O)N(C)CC23CCCC3)nc2nc(Nc3ccc(N(C)C(=O)CN(C)C)cc3)ncc12]

General Procedure 1 was applied to 2-(dimethylamino)-N-methyl-N-{4-[(7-{3-methyl-2-oxo-1,3-diazaspiro [4.4]nonan-1-yl}-5-[2-(triisopropylsilyl)ethynyl]pyrido[2,3-d]pyrimidin-2-yl)amino]phenyl}acetamide (150 mg, 0.224 mmol) and potassium fluoride (130 mg, 2.24 mmol) in THF (0.50 mL), DMF (0.50 mL) and water (0.10 mL), stirred for 1 hour at 60° C. The residue was purified by reverse phase flash column chromatography eluting with acetonitrile (10-50%) in water (0.1% formic acid), to yield the title compound as a light yellow solid (26.0 mg, 22%).

(ES, m/z) [M+H]⁺=513.25

¹H-NMR (400 MHz, DMSO-d₆) δ 10.26 (s, 1H), 9.19 (s, 1H), 8.33 (s, 1H), 8.06 (d, J=8.0 Hz, 2H), 7.26 (d, J=8.3 Hz, 2H), 5.00 (s, 1H), 3.36 (s, 3H), 3.15 (s, 2H), 2.94 (s, 2H), 2.84 (s, 3H), 2.64 (dt, J=11.7, 6.0 Hz, 2H), 2.26 (s, 2H), 2.19 (s, 6H), 1.70-1.59 (m, 4H).

### Example 107

### 1-[2-({4-[2-(Dimethylamino)ethoxy]phenyl}amino)-5-[2-(triisopropylsilyl)ethynyl]pyrido[2,3-d]pyrimidin-7-yl]-3-methyl-1,3-diazaspiro[4.4]nonan-2-one

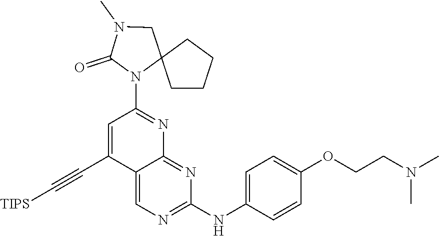

General Procedure 3 was applied to 1-{2-methanesulfinyl-5-[2-(triisopropylsilyl)ethynyl]pyrido[2,3-d]pyrimidin-7-yl}-3-methyl-1,3-diazaspiro[4.4]nonan-2-one (200 mg, 0.380 mmol), 4-[2-(dimethylamino)ethoxy]aniline (82.3 mg, 0.456 mmol) and trifluoroacetic acid (86.7 mg, 0.760 mmol) in 2-butanol (5.0 mL) was stirred for 2 hours at 80° C. The resulting mixture was diluted with water (10 mL) and extracted with ethyl acetate (3×10 mL). The combined organic layers were dried (Na₂SO₄) and concentrated under reduced pressure to yield the title compound as a yellow solid (166 mg, 68%).

### 1-[2-({4-[2-(Dimethylamino)ethoxy]phenyl}amino)-5-ethynyl)pyrido[2,3-d]pyrimidin-7-yl]-3-methyl-1,3-diazaspiro[4.4]nonan-2-one

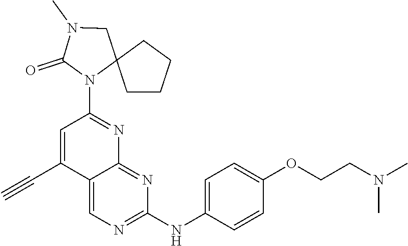 [SMILES: C#Cc1cc(N2C(=O)N(C)CC23CCCC3)nc2nc(Nc3ccc(OCCN(C)C)cc3)ncc12]

General Procedure 1 was applied to 1-[2-({4-[2-(dimethylamino)ethoxy]phenyl}amino)-5-[2-(triisopropylsilyl)ethynyl]pyrido[2,3-d]pyrimidin-7-yl]-3-methyl-1,3-diazaspiro[4.4]nonan-2-one (150 mg, 0.234 mmol) and potassium fluoride (136 mg, 2.34 mmol) in THF (0.50 mL), DMF (0.50 mL) and water (0.10 mL), stirred for 1 hour at 60° C. The residue was purified by reverse phase flash column chromatography eluting with acetonitrile (10-50%) in water (0.1% formic acid), to yield the title compound as a yellow solid (22.0 mg, 19%).

(ES, m/z) [M+H]⁺=486.35

¹H-NMR (400 MHz, DMSO-d₆) δ 9.96 (s, 1H), 9.13 (s, 1H), 8.28 (s, 1H), 7.87 (s, 2H), 6.93-6.85 (m, 2H), 4.99 (s, 1H), 4.02 (t, J=5.8 Hz, 2H), 3.36 (s, 2H), 2.84 (s, 3H), 2.61 (q, J=5.7 Hz, 4H), 2.26 (s, 2H), 2.22 (s, 6H), 1.64 (t, J=7.1 Hz, 4H).

### Example 108

### 1-{2-[(4-Methanesulfonyl)phenyl)amino]-5-[2-(triisopropylsilyl)ethynyl]pyrido[2,3-d]pyrimidin-7-yl}-3-methyl-1,3-diazaspiro[4.4]nonan-2-one

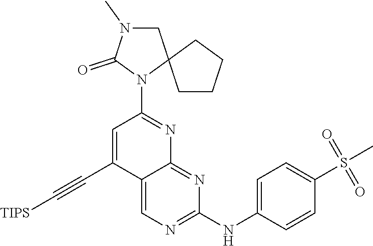

General Procedure 3 was applied to 1-{2-methanesulfinyl-5-[2-(triisopropylsilyl)ethynyl]pyrido[2,3-d]pyrimidin-7-yl}-3-methyl-1,3-diazaspiro[4.4]nonan-2-one (200 mg, 0.380 mmol), 4-methylsulfonylaniline (78.1 mg, 0.456 mmol) and trifluoroacetic acid (86.7 mg, 0.760 mmol) in 2-butanol (5.0 mL), stirred for 2 hours at 80° C. The resulting mixture was diluted with water (6.0 mL) and extracted with ethyl acetate (3×10 mL). The combined organic layers were dried (Na₂SO₄) and concentrated under reduced pressure to yield the title compound as a yellow solid (185 mg, 77%).

### 1-{5-Ethynyl-2-[(4-methanesulfonylphenyl)amino]pyrido [2,3-d]pyrimidin-7-yl}-3-methyl-1,3-diazaspiro [4.4]nonan-2-one

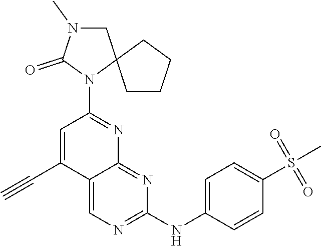 [SMILES: C#Cc1cc(N2C(=O)N(C)CC23CCCC3)nc2nc(Nc3ccc(S(C)(=O)=O)cc3)ncc12]

General Procedure 1 was applied to 1-{2-[(4-methanesulfonylphenyl)amino]-5-[2-(triisopropylsilyl) ethynyl]pyrido[2,3-d]pyrimidin-7-yl}-3-methyl-1,3-diazaspiro[4.4]nonan-2-one (150 mg, 0.237 mmol) and potassium fluoride (138 mg, 2.37 mmol) in THF (0.50 mL), DMF (0.50 mL) and water (0.10 mL), stirred for 1 hour at 60° C. The residue was purified by reverse phase flash column chromatography eluting with acetonitrile (10-50%) in water (0.1% formic acid), to yield the title compound as a light yellow solid (17.0 mg, 15%).

(ES, m/z) [M+H]⁺=477.20

¹H-NMR (400 MHz, DMSO-d₆) δ 10.59 (s, 1H), 9.21 (s, 1H), 8.37 (s, 1H), 8.25 (d, J=8.8 Hz, 2H), 7.88-7.78 (m, 2H), 5.00 (s, 1H), 3.34 (s, 2H), 3.18 (s, 3H), 2.83 (s, 3H), 2.62 (dd, J=14.4, 5.6 Hz, 2H), 2.28 (t, J=6.9 Hz, 2H), 1.75-1.58 (m, 4H).

### Example 109

### N,N-Dimethyl-4-[(7-{3-methyl-2-oxo-1,3-diazaspiro[4.4]nonan-1-yl}-5-[2-(triisopropylsilyl)ethynyl]pyrido[2,3-d]pyrimidin-2-yl)amino]benzenesulfonamide

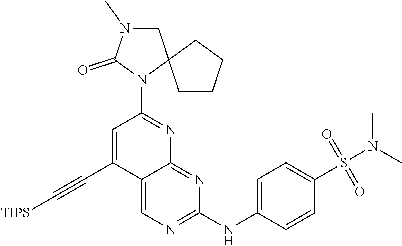

General Procedure 3 was applied to 1-{2-methanesulfinyl-5-[2-(triisopropylsilyl)ethynyl]pyrido[2,3-d]pyrimidin-7-yl}-3-methyl-1,3-diazaspiro[4.4]nonan-2-one (200 mg, 0.380 mmol), N¹-dimethylsulfanilamide (91.4 mg, 0.456 mmol) and trifluoroacetic acid (86.7 mg, 0.760 mmol) in 2-butanol (5.0 mL), stirred for 2 hours at 80° C. The resulting mixture was diluted with water (10 mL) and extracted with ethyl acetate (3×10 mL). The combined organic layers were dried (Na₂SO₄) and concentrated under reduced pressure to yield the title compound as a yellow solid (171 mg, 66%).

### 4-[(5-Ethynyl-7-{3-methyl-2-oxo-1,3-diazaspiro[4.4]nonan-1-yl}pyrido[2,3-d]pyrimidin-2-yl)amino]-N,N-dimethylbenzenesulfonamide

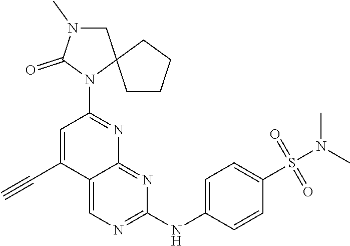 [SMILES: C#Cc1cc(N2C(=O)N(C)CC23CCCC3)nc2nc(Nc3ccc(S(=O)(=O)N(C)C)cc3)ncc12]

General Procedure 1 was applied to N, N-dimethyl-4-[(7-{3-methyl-2-oxo-1,3-diazaspiro[4.4]nonan-1-yl}-5-[2-(triisopropylsilyl)ethynyl]pyrido[2,3-d]pyrimidin-2-yl)amino]benzenesulfonamide (150 mg, 0.227 mmol) and potassium fluoride (131 mg, 2.27 mmol) in THF (0.50 mL), DMF (0.50 mL) and water (0.10 mL), stirred for 1 hour at 60° C. The residue was purified by reverse phase flash column chromatography eluting with acetonitrile (10-50%) in water (0.1% formic acid), to yield the title compound as a light yellow solid (12.0 mg, 10%).

(ES, m/z) [M+H]⁺=506.20

¹H-NMR (400 MHz, DMSO-d₆) δ 10.55 (s, 1H), 9.17 (s, 1H), 8.33 (s, 1H), 8.19 (d, J=8.5 Hz, 2H), 7.61 (d, J=8.5 Hz, 2H), 4.97 (s, 1H), 3.32 (s, 3H), 2.78 (s, 3H), 2.65-2.55 (m, 2H), 2.50-2.36 (m, 6H), 2.22 (d, J=8.8 Hz, 2H), 1.69-1.49 (m, 4H).

### Example 110

### N-[2-(Dimethylamino)ethyl]-N-methyl-4-[(7-{3-methyl-2-oxo-1,3-diazaspiro[4.4]nonan-1-yl}-5-[2-(triisopropylsilyl)ethynyl]pyrido[2,3-d]pyrimidin-2-yl)amino]benzenesulfonamide

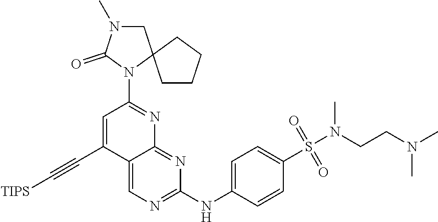

General Procedure 3 was applied to 1-{2-methanesulfinyl-5-[2-(triisopropylsilyl)ethynyl]pyrido[2,3-d]pyrimidin-7-yl}-1,3-diazaspiro[4.4]nonan-2-one (180 mg, 0.352 mmol), N-(2-(dimethylamino)ethyl)-N-methyl-4-aminobenzenesulfonamide and trifluoroacetic acid (50.0 μL, 0.653 mmol) in 2-methyl-2-butanol (2.0 mL), stirred overnight at 110° C. The resulting mixture was diluted with water (10 mL) and extracted with ethyl acetate (2×10 mL). The resulting mixture was concentrated under reduced pressure. The residue was purified by reverse phase flash column chromatography eluting with acetonitrile (10-100%) in water (0.1% formic acid), to yield the title compound as a yellow solid (80.0 mg, 31%).

### N-[2-(Dimethylamino)ethyl]-4-[(5-ethynyl-7-{3-methyl-2-oxo-1,3-diazaspiro[4.4]nonan 1-yl}pyrido[2,3-d]pyrimidin-2-yl)amino]-N-methylbenzenesulfonamide

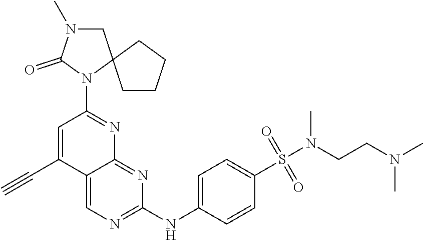 [SMILES: C#Cc1cc(N2C(=O)N(C)CC23CCCC3)nc2nc(Nc3ccc(S(=O)(=O)N(C)CCN(C)C)cc3)ncc12]

General Procedure 1 was applied to N-[2-(dimethylamino)ethyl]-N-methyl-4-[(7-{3-methyl-2-oxo-1,3-diazaspiro[4.4]nonan-1-yl}-5-[2-(triisopropylsilyl)ethynyl]pyrido[2,3-d]pyrimidin-2-yl)amino]benzenesulfonamide (80.0 mg, 0.111 mmol and potassium fluoride (32.3 mg, 0.555 mmol) in THF (1.0 mL), DMF (1.0 mL) and water (0.50 mL), stirred for 1 hour at room temperature. The residue was purified by flash column chromatography eluting with methanol (15%) in dichloromethane to afford the title compound as a light yellow solid (12.0 mg, 19%).

(ES, m/z) [M+H]⁺=563.05

¹H-NMR (400 MHz, DMSO-d₆) δ 10.60 (s, 1H), 9.25 (s, 1H), 8.40 (s, 1H), 8.27-8.20 (m, 2H), 7.74-7.68 (m, 2H), 5.05 (s, 1H), 3.02 (t, J=6.7 Hz, 2H), 2.86 (s, 3H), 2.68 (s, 3H), 2.37 (t, J=6.7 Hz, 2H), 2.32-2.25 (m, 2H), 2.14 (s, 6H), 1.71-1.62 (m, 4H).

### Example 111

### 2-Amino-2-cyclopentylethanol

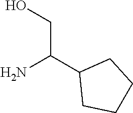

A solution of cyclopentylglycine (3.00 g, 20.9 mmol, 1.0 eq.) and lithium aluminum hydride (950 mg, 25.1 mmol, 1.2 eq.) in TH F (50 mL) was stirred for 4 hours at room temperature under nitrogen atmosphere. The reaction was quenched by the addition of water (10 mL) at 0° C. The precipitated solids were collected by filtration and washed with THF (50 mL). The resulting mixture was concentrated under reduced pressure to yield the title compound as light yellow oil (2.50 g, 92%).

### 4-Cyclopentyl-1, 3-oxazolidin-2-one

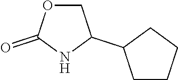 [SMILES: O=C1NC(C2CCCC2)CO1]

A solution of 2-amino-2-cyclopentylethanol (1.50 g, 11.6 mmol, 1.0 eq.), triphosgene (5.17 g, 17.4 mmol, 1.5 eq.) and hydrochloric acid (6M) (3.53 mL, 116 mmol, 10 eq.) in water (10 mL) and THF (10 mL) was stirred for 2 hours at room temperature under air atmosphere. The resulting mixture was diluted with ethyl acetate (20 mL). The aqueous layer was extracted with ethyl acetate (10 mL) and concentrated under reduced pressure. The residue was purified by flash column chromatography eluting with ethyl acetate (16%) in 40-60 petroleum ether to afford the title compound as a colorless solid (400 mg, 22%).

### 4-Cyclopentyl-3-[2-(methylsulfanyl)-5-[2-(triisopropylsilyl)ethynyl]pyrido[2,3-d]pyrimidin-7-yl]-1,3-oxazolidin-2-one

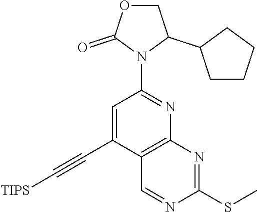

A solution of 7-chloro-2-(methylsulfanyl)-5-[2-(triisopropylsilyl)ethynyl]pyrido[2,3-d]pyrimidine (620 mg, 1.58 mmol, 1.0 eq.), 4-cyclopentyl-1,3-oxazolidin-2-one (294 mg, 1.89 mmol, 1.2 eq.), tris(dibenzyldeneacetone)dipalladium (0) (72.4 mg, 79.0 μmol, 0.05 eq.), XantPhos (91.5 mg, 0.158 mmol, 0.10 eq.) and potassium carbonate (437 mg, 3.16 mmol, 2.0 eq.) in dioxane (10 mL) was stirred for 1 hour at 100° C. under nitrogen atmosphere. The mixture was allowed to cool down to room temperature then concentrated under reduced pressure. The residue was purified by flash column chromatography eluting with ethyl acetate (10%) in 40-60 petroleum ether to afford the title compound as a red solid (482 mg, 59%).

### 4-Cyclopentyl-3-{2-methanesulfonyl-5-[2-(triisopropylsilyl)ethynyl]pyrido[2,3-d]pyrimidin-7-yl}-1.3-oxazolidin-2-one

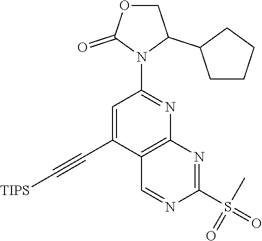

General Procedure 2 was applied to 4-cyclopentyl-3-[2-(methylsulfanyl)-5-[2-(triisopropylsilyl)ethynyl]pyrido[2,3-d]pyrimidin-7-yl]-1,3-oxazolidin-2-one (500 mg, 0.979 mmol) and m-CPBA (371 mg, 2.15 mmol) in dichloromethane (10 mL) to yield the title compound as a light yellow solid (497 mg, 93%).

### 4-Cyclopentyl-3-(2-{[4-(4-methylpiperazin-1-yl)phenyl]amino}-5-[2-(triisopropylsilyl)ethynyl]pyrido[2,3-d]pyrimidin-7-yl)-1,3-oxazolidin-2-one

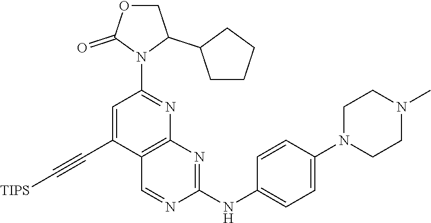

General Procedure 3 was applied to 4-cyclopentyl-3-{2-methanesulfonyl-5-[2-(triisopropylsilyl)ethynyl]pyrido[2,3-d]pyrimidin-7-yl}-1,3-oxazolidin-2-one (497 mg, 0.916 mmol), 4-(4-methylpiperazin-1-yl)aniline (227 mg, 1.19 mmol) and trifluoroacetic acid (0.140 mL, 1.83 mmol) in 2-methyl-2-butanol (8.0 mL) was stirred for 1 hour at 100° C. The residue was purified by reverse flash column chromatography eluting with acetonitrile (10-80%) in water (0.1% formic acid) to yield the title compound as a red oil (150 mg, 25%).

### 4-Cyclopentyl-3-(5-ethynyl-2-{[4-(4-methylpiperazin-1-yl)phenyl]amino}pyrido[2,3-d]pyrimidin-7-yl)-1,3-oxazolidin-2-one

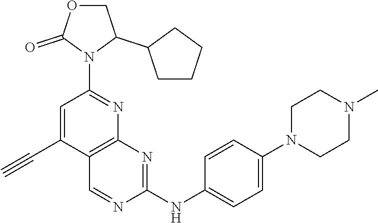 [SMILES: C#Cc1cc(N2C(=O)OCC2C2CCCC2)nc2nc(Nc3ccc(N4CCN(C)CC4)cc3)ncc12]

General procedure xx was applied to 4-cyclopentyl-3-(2-{[4-(4-methylpiperazin-1-yl)phenyl]amino}-5-[2-(triisopropylsilyl)ethynyl]pyrido[2,3-d]pyrimidin-7-yl)-1,3-oxazolidin-2-one (80.0 mg, 0.122 mmol) and potassium fluoride (71.1 mg, 1.22 mmol) in THF (2.0 mL), DMF (2.0 mL) and water (0.5 mL) was stirred for 1 hour at 60° C. The residue was purified by reverse phase flash column chromatography eluting with acetonitrile (10-50%) in water (0.1% formic acid), to yield the title compound as an orange solid (10.0 mg, 16%).

(ES, m/z): [M+H]⁺=498.15

¹H-NMR (300 MHz, DMSO-d₆) δ 10.05 (s, 1H), 9.22 (s, 1H), 8.14 (s, 1H), 7.71 (s, 2H), 6.97 (m, 2H), 5.12 (d, J=5.1 Hz, 2H), 4.55 (t, J=8.6 Hz, 1H), 4.35 (dd, J=9.0, 2.8 Hz, 1H), 3.10 (t, J=5.0 Hz, 4H), 2.70 (s, 1H), 2.46 (t, J=5.0 Hz, 4H), 2.23 (s, 3H), 1.74-1.58 (m, 3H), 1.56-1.41 (m, 3H), 1.26 (d, J=14.1 Hz, 2H).

### Example 112

### 2-Amino-3-cyclopentylpropan-1-ol

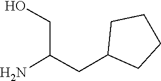

A solution of 2-amino-3-cyclopentylpropanoic acid (1.00 g, 6.36 mmol, 1.0 eq.) and lithium aluminium hydride (240 mg, 6.36 mmol, 1.0 eq.) in THF (10 mL) was stirred for 30 minutes at room temperature under air atmosphere. The reaction was quenched with water at 0° C. The aqueous layer was extracted with ethyl acetate (3×30 mL). The combined organic layers were concentrated under reduced pressure to yield the title compound as a light yellow oil (872 mg, 96%).

### 3-Cyclopentyl-2-{[2-(methylsulfanyl)-5-[2-(triisopropylsilyl)ethynyl]pyrido[2,3-d]pyrimidin-7-yl]amino}propan-1-ol

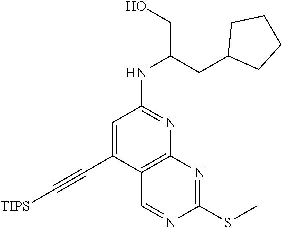

A solution of 7-chloro-2-(methylsulfanyl)-5-[2-(triisopropylsilyl)ethynyl]pyrido[2,3-d]pyrimidine (1.20 g, 3.06 mmol, 1.0 eq.), 2-amino-3-cyclopentylpropan-1-ol (570 mg, 3.979 mmol, 1.3 eq.), tris(dibenzyldeneacetone)dipalladium (0) (140 mg, 0.153 mmol, 0.05 eq.), XantPhos (180 mg, 0.306 mmol, 0.1 eq.) and potassium carbonate (850 mg, 6.12 mmol, 2.0 eq.) in dioxane (20 mL) was stirred for 1 hour at 100° C. The mixture was allowed to cool down to room temperature then concentrated under reduced pressure. The residue was purified by flash column chromatography eluting with ethyl acetate (35%) in 40-60 petroleum ether to afford the title compound as a light yellow solid (804 mg, 52%).

### 4-(Cyclopentylmethyl)-3-[2-(methylsulfanyl)-5-[2-(triisopropylsilyl)ethynyl]pyrido[2,3-d]pyrimidin-7-yl]-1,3-oxazolidin-2-one

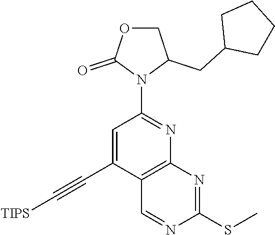

A solution of 3-cyclopentyl-2-{[2-(methylsulfanyl)-5-[2-(triisopropylsilyl)ethynyl]pyrido[2,3-d]pyrimidin-7-yl]amino}propan-1-ol (800 mg, 1.60 mmol, 1.0 eq.), diisopropylethylamine (414 mg, 3.21 mmol, 2.0 eq.) and CDI (390. mg, 2.41 mmol, 1.5 eq.) in DMF (10 mL) was stirred for 1 hour at 60° C. The mixture was allowed to cool down to room temperature then diluted with water (100 mL). The aqueous layer was extracted with ethyl acetate (2×20 mL), the combined organic layers dried (MgSO₄) and concentrated under reduced pressure. The residue was purified by flash column chromatography eluting with ethyl acetate (10%) in 40-60 petroleum ether to yield the title compound as a light yellow solid (560 mg, 66%) as a light yellow solid.

### 4-(Cyclopentylmethyl)-3-{2-methanesulfinyl-5-[2-(triisopropylsilyl)ethynyl]pyrido[2,3-d]pyrimidin-7-yl}-1,3-oxazolidin-2-one

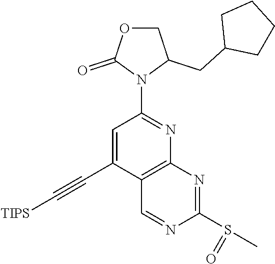

General Procedure 2 was applied to 4-(cyclopentylmethyl)-3-[2-(methylsulfanyl)-5-[2-(triisopropylsilyl)ethynyl]pyrido[2,3-d]pyrimidin-7-yl]-1,3-oxazolidin-2-one (530 mg, 1.01 mmol) and m-CPBA (174 mg, 1.01 mmol) in dichloromethane (10 mL) to yield the title compound as a light yellow solid (530 mg, 97%).

### 4-(Cyclopentylmethyl)-3-(2-{[4-(4-methylpiperazin-1-yl)phenyl]amino}-5-[2-(triisopropylsilyl)ethynyl]pyrido[2,3-d]pyrimidin-7-yl)-1,3-oxazolidin-2-one

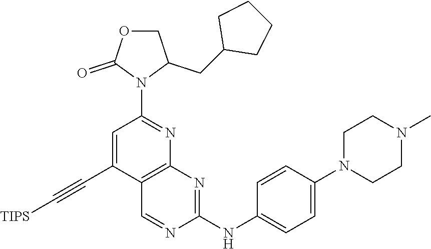

General procedure SNAr was applied to 4-(cyclopentylmethyl)-3-{2-methanesulfonyl-5-[2-(triisopropylsiyl)ethynyl]pyrido[2,3-d]pyrimidin-7-yl}-1, 3-oxazolidin-2-one (500 mg, 0.898 mmol), 4-(4-methylpiperazin-1-yl)aniline (223 mg, 1.17 mmol) and trifluoroacetic acid (0.130 mL, 1.79 mmol) in 2-methyl-2-butanol (8.0 mL) was stirred for 1 hour at 100° C. The residue was purified by reverse phase flash column chromatography eluting with acetonitrile (10-80%) in water(0.1% formic acid) to yield the title compound as a light yellow solid (207 mg, 34%).

### 4-(Cyclopentylmethyl)-3-(5-ethynyl-2-{[4-(4-methylpiperazin-1-yl)phenyl]amino}pyrido[2,3-d]pyrimidin-7-yl)-1,3-oxazolidin-2-one

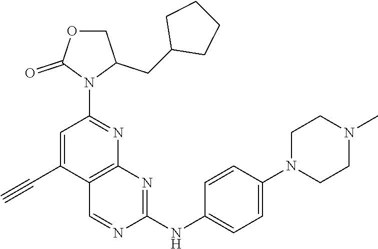 [SMILES: C#Cc1cc(N2C(=O)OCC2CC2CCCC2)nc2nc(Nc3ccc(N4CCN(C)CC4)cc3)ncc12]

General Procedure 1 was applied to 4-(cyclopentylmethyl)-3-(2-{[4-(4-methylpiperazin-1-yl)phenyl]amino}-5-[2-(triisopropylsilyl)ethynyl]pyrido[2,3-d]pyrimidin-7-yl)-1,3-oxazolidin-2-one (100 mg, 0.150 mmol) and potassium fluoride (86.9 mg, 1.50 mmol) in THF (2.0 mL), DMF (2.0 mL) and water (0.5 mL) was stirred for 1 hour at 60° C. The residue was purified by reverse phase flash column chromatography eluting with acetonitrile (10-60%) in water (0.1% formic acid), to yield the title compound as a yellow solid (20.0 mg, 26%).

(ES, m/z) [M+H]⁺=512.05

¹H-NMR (300 MHz, DMSO-d₆) δ 10.06 (s, 1H), 9.21 (s, 1H), 8.13 (s, 1H), 7.85 (s, 2H), 6.94-6.82 (m, 2H), 5.11 (s, 1H), 4.93 (s, 1H), 4.60 (t, J=8.2 Hz, 1H), 4.34 (dd, J=8.5, 2.8 Hz, 1H), 3.09 (t, J=4.9 Hz, 4H), 2.46 (d, J=5.2 Hz, 4H), 2.23 (s, 3H), 1.99 (d, J=11.1 Hz, 1H), 1.93-1.45 (m, 9H), 1.09 (s, 1H).

### Example 113

### 3-Methyl-1-{[2-(methylsulfanyl)-5-[2-(triisopropylsilyl)ethynyl]pyrido[2,3-d]pyrimidin-7-yl]amino}butan-2-ol

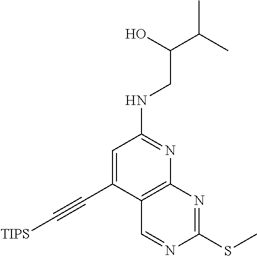

A solution of 7-chloro-2-(methylsulfanyl)-5-[2-(triisopropylsilyl)ethynyl]pyrido[2,3-d]pyrimidine (1.00 g, 2.55 mmol, 1.0 eq.), 1-amino-3-methylbutan-2-ol (320 mg, 3.06 mmol, 1.2 eq.), XantPhos (70.0 mg, 0.128 mmol, 0.05 eq.), tris(dibenzyldeneacetone)dipalladium (0) (230 mg, 0.255 mmol, 0.10 eq.) and potassium carbonate (710 mg, 5.10 mmol, 2 eq.) in dioxane (10 mL) was stirred for 1 hour at 100° C. under nitrogen atmosphere. The mixture was allowed to cool down to room temperature then concentrated under reduced pressure. The residue was purified by flash column chromatography eluting with ethyl acetate (25%) in 40-60 petroleum ether to afford the title compound as a yellow solid (450 mg, 38%).

### 5-Isopropyl-3-[2-(methylsulfanyl)-5-[2-(triisopropylsilyl)ethynyl]pyrido[2,3-d]pyrimidin-7-yl]-1,3-oxazolidin-2-one

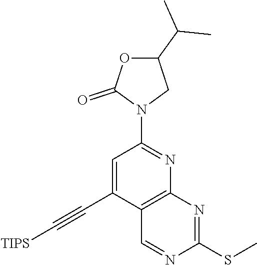

A solution of 3-methyl-1-{[2-(methylsulfanyl)-5-[2-(triisopropylsilyl)ethynyl]pyrido[2,3-d]pyrimidin-7-yl]amino}butan-2-ol (450 mg, 0.981 mmol, 1.0 eq.), CDI (238 mg, 1.47 mmol, 1.5 eq.) and diisopropylethylamine (253 mg, 1.96 mmol, 2.0 eq.) in DMF (8.0 mL) was stirred for 1 hour at 60° C. The mixture was allowed to cool down to room temperature. The residue was purified by reverse phase flash column chromatography eluting with acetonitrile (10-60%) in water (0.1% formic acid) to yield the title compound as a yellow solid (350 mg, 73%).

### 5-Isopropyl-3-{2-methanesulfonyl-5-[2-(triisopropylsilyl)ethynyl]pyrido[2,3-d]pyrimidin-7-yl}-1,3-oxazolidin-2-one

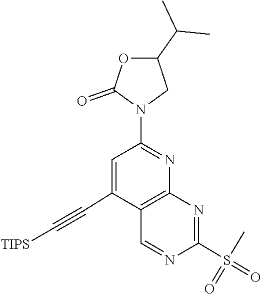

General Procedure 2 was applied to 5-isopropyl-3-[2-(methylsulfanyl)-5-[2-(triisopropylsilyl)ethynyl]pyrido[2,3-d]pyrimidin-7-yl]-1,3-oxazolidin-2-one (350 mg, 0.722 mmol) and m-CPBA (274 mg, 1.59 mmol) in dichloromethane (5.0 mL). The residue was purified by flash column chromatography eluting with methanol (10%) in dichloromethane to afford the title compound as a reddish brown yellow solid (335 mg, 89%). 5-Isopropyl-3-(2-{[4-(4-methylpiperazin-1-yl)phenyl]amino}-5-[2-(triisopropylsilyl)ethynyl]pyrido[2,3-d]pyrimidin-7-yl)-1,3-oxazolidin-2-one

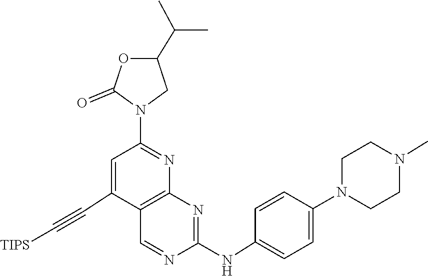

General Procedure 3 was applied to 5-isopropyl-3-{2-methanesulfonyl-5-[2-(triisopropylsilyl)ethynyl]pyrido[2,3-d]pyrimidin-7-yl}-1,3-oxazolidin-2-one (320 mg, 0.619 mmol), 4-(4-methylpiperazin-1-yl)aniline (154 mg, 0.805 mmol) and trifluoroacetic acid (90.0 μL, 1.24 mmol) in 2-methyl-2-butanol (5.0 mL), stirred for 1 hour at 100° C. The residue was purified by reverse phase flash column chromatography eluting with acetonitrile (10-80%) in water (0.1% formic acid), to yield the title compound as a reddish brown yellow solid (290 mg, 74%).

### 3-(5-Ethynyl-2-{[4-(4-methylpiperazin-1-yl)phenyl]amino}pyrido[2,3-d]pyrimidin-7-yl)-5-isopropyl-1.3-oxazolidin-2-one

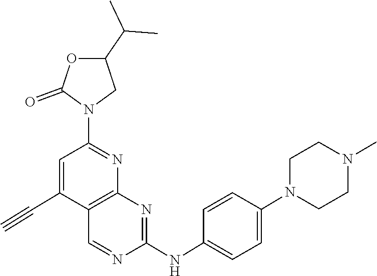 [SMILES: C#Cc1cc(N2CC(C(C)C)OC2=O)nc2nc(Nc3ccc(N4CCN(C)CC4)cc3)ncc12]

General Procedure 1 was applied to 5-isopropyl-3-(2-{[4-(4-methylpiperazin-1-yl)phenyl]amino}-5-[2-(triisopropylsilyl)ethynyl]pyrido[2,3-d]pyrimidin-7-yl)-1,3-oxazolidin-2-one (170 mg, 0.271 mmol) and potassium fluoride (157 mg, 2.71 mmol) in THF (2.0 mL), DMF (2.0 mL) and water (0.5 mL), stirred for 1 hour at 60° C. The residue was purified by reverse phase flash column chromatography eluting with acetonitrile (10-50%) in water (0.1% formic acid), to yield the title compound as an orange solid (30.0 mg, 23%).

(ES, m/z): [M+H]⁺=472.15

¹H-NMR (300 MHz, DMSO-d₆) δ 9.18 (d, J=1.0 Hz, 1H), 8.14 (d, J=1.0 Hz, 1H), 7.71 (d, J=8.4 Hz, 2H), 7.01 (d, J=8.8 Hz, 2H), 4.85 (d, J=2.1 Hz, 1H), 4.47 (t, J=7.6 Hz, 1H), 4.30 (t, J=9.5 Hz, 1H), 3.88 (s, 1H), 3.75 (d, J=13.2 Hz, 2H), 3.49 (d, J=12.2 Hz, 3H), 3.13 (d, J=12.9 Hz, 2H), 2.94 (d, J=11.9 Hz, 2H), 2.83 (s, 3H), 1.99-1.84 (m, 1H), 0.93 (dd, J=9.5, 6.7 Hz, 6H).

### Example 114

### (1-{[2-(Methylsulfanyl)-5-[2-(triisopropylsilyl)ethnyl]pyrido[2,3-d]pyrimidin-7-yl]amino}cyclopentyl)methanol

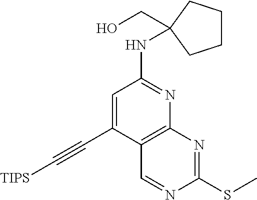

To a solution of 7-chloro-2-(methylsulfanyl)-5-[2-(triisopropylsilyl)ethynyl]pyrido[2,3-d]pyrimidine (1.50 g, 3.82 mmol, 1.0 eq.) and 1-amino-cyclopentanemethanol (480 mg, 4.21 mmol, 1.1 eq.) in dioxane (6.0 mL) were added potassium carbonate (1.06 g, 7.65 mmol, 2.0 eq.), tris(dibenzyldeneacetone)dipalladium (0) (180 mg, 0.191 mmol, 0.05 eq.) and XantPhos (220 g, 0.383 mmol, 0.1 eq.). The reaction was stirred for 2 hours at 100° C. then cooled to room temperature. The resulting mixture was filtered, washing with dichloromethane (3×10 mL). The filtrate was concentrated under reduced pressure. The residue was purified by flash column chromatography eluting with ethyl acetate (10%) in 40-60 petroleum ether to yield the title compound as a brown solid (540 mg, 30%).

¹H-NMR (300 MHz, DMSO-d₆) δ 8.83 (s, 1H), 7.76 (s, 1H), 7.05 (s, 1H), 5.12 (t, J=5.7 Hz, 1H), 3.72 (d, J=5.8 Hz, 2H), 2.55 (s, 3H), 2.06-1.94 (m, 2H), 1.82 (dt, J=12.5, 6.0 Hz, 2H), 1.64 (dd, J=24.8, 7.0 Hz, 4H), 1.12 (d, J=4.9 Hz, 21H).

### 1-[2-(Methylsulfanyl)-5-[2-(triisopropylsilyl)ethynyl]pyrido[2,3-d]pyrimidin-7-yl]-3-oxa

### 1-azaspiro[4.4]nonan-2-one

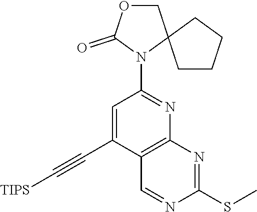

A solution of (1-{[2-(methylsulfanyl)-5-[2-(triisopropylsilyl)ethynyl]pyrido[2,3-d]pyrimidin-7-yl]amino}cyclopentyl)methanol (540 mg, 1.15 mmol, 1.0 eq.), CDI (558 mg, 3.44 mmol, 3.0 eq.) and diisopropylethylamine (445 mg, 3.44 mmol, 3.0 eq.) in DMF (3.0 mL) was stirred for 2 hours at 80° C. The mixture was allowed to cool down to room temperature then quenched with water (20 mL) and extracted with dichloromethane (3×30 mL). The combined organic layers were washed with brine (2×20 mL), dried (Na₂SO₄) and concentrated under reduced pressure. The residue was purified by flash column chromatography eluting with ethyl acetate (5%) in 40-60 petroleum ether to afford the title compound as a white solid (400 mg, 70%).

### 1-{2-Methanesulfonyl-5-[2-(triisopropylsilyl)ethynyl]pyrido[2,3-d]pyrimidin-7-yl}-3-oxa 1-azaspiro[4.4]nonan-2-one

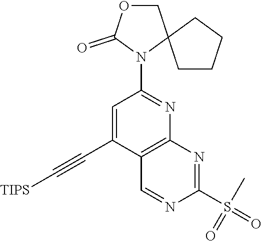

General Procedure 2 was applied to 1-[2-(methylsulfanyl)-5-[2-(triisopropylsilyl)ethynyl]pyrido[2,3-d]pyrimidin-7-yl]-3-oxa-1-azaspiro[4.4]nonan-2-one (400 mg, 0.805 mmol) and m-CPBA (347 mg, 2.01 mmol) in dichloromethane (3.0 mL) to yield the title compound as a light yellow solid (423 mg, 99%).

### 1-(2-{[4-(4-Methylpiperazin-1-yl)phenyl]amino}-5-[2-(triisopropylsilyl)ethynyl]pyrido[2,3-d]pyrimidin-7-yl)-3-oxa-1-azaspiro[4.4]nonan-2-one

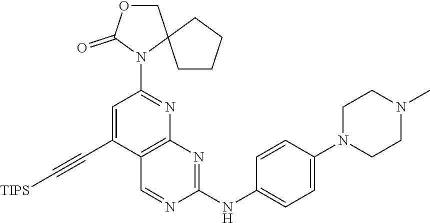

General Procedure 3 was applied to 1-{2-methanesulfonyl-5-[2-(triisopropylsilyl)ethynyl]pyrido[2,3-d]pyrimidin-7-yl}-3-oxa-1-azaspiro[4.4]nonan-2-one (338 mg, 0.639 mmol), trifluoroacetic acid (145 mg, 1.28 mmol) and 4-(4-methylpiperazin-1-yl)aniline (159 mg, 0.831 mmol) in butan-2-ol (2.0 mL), stirred for 1 hour at 100° C. The residue was purified by Prep-HPLC eluting with acetonitrile (20-70%) in water (0.05% trifluoroacetic acid), to yield the title compound as a reddish brown solid (359 mg, 88%).

¹H-NMR (300 MHz, DMSO-d₆) δ 10.07 (s, 1H), 9.87 (s, 1H), 9.17 (s, 1H), 7.96 (s, 1H), 7.86 (s, 1H), 7.05-6.95 (m, 2H), 4.32 (s, 2H), 3.79 (d, J=13.0 Hz, 2H), 3.28-3.09 (m, 2H), 2.90 (d, J=13.8 Hz, 5H), 2.58 (q, J=7.3, 6.5 Hz, 2H), 2.23 (s, 2H), 1.71 (dd, J=20.1, 10.3 Hz, 4H), 1.24-1.12 (m, 21H).

### 1-(5-Ethynyl-2-{[4-(4-methylpiperazin-1-yl)phenyl]amino}pyrido[2,3-d]pyrimidin-7-yl)-3-oxa-1-azaspiro[4.4]nonan-2-one

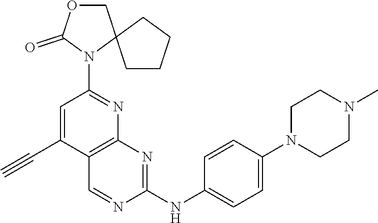 [SMILES: C#Cc1cc(N2C(=O)OCC23CCCC3)nc2nc(Nc3ccc(N4CCN(C)CC4)cc3)ncc12]

General Procedure 1 was applied to 1-(2-{[4-(4-methylpiperazin-1-yl)phenyl]amino}-5-[2-(triisopropylsilyl)ethynyl]pyrido[2,3-d]pyrimidin-7-yl)-3-oxa-1-azaspiro[4.4]nonan-2-one (150 mg, 0.234 mmol) and potassium fluoride (136 mg, 2.34 mmol) in THF (2.0 mL) and DMF (1.0 mL), stirred for 1 hour at 60° C. The residue was purified by Prep-HPLC eluting with methanol (20-70%) in water (0.1% ammonium acetate), to yield the title compound as a reddish brown solid (28.0 mg, 25%).

(ES, m/z): [M+H]⁺=484.15

¹H-NMR (400 MHz, DMSO-d₆) δ 10.03 (s, 1H), 9.21 (s, 1H), 7.91 (d, J=53.9 Hz, 3H), 6.98-6.86 (m, 2H), 5.08 (s, 1H), 4.31 (s, 2H), 3.09 (t, J=5.0 Hz, 4H), 2.62-2.55 (m, 2H), 2.45 (t, J=5.0 Hz, 4H), 2.22 (s, 5H), 1.81-1.55 (m, 4H).

### Example 115

### Tert-butyl N-[(1S)-1-cyclopentyl-2-hydroxyethyl]carbamate

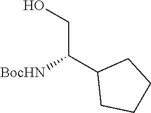 [SMILES: CC(C)(C)OC(=O)N[C@H](CO)C1CCCC1]

A solution of (S)-[(tert-butoxycarbonyl)amino](cyclopentyl)acetic acid (2.00 g, 8.22 mmol, 1.0 eq.), diethyl ether (20 mL) and lithium aluminium hydride (310 mg, 8.22 mmol, 1.0 eq.) in THF (0.33 mL) was stirred for 30 minutes at 0° C. The resulting mixture was diluted with dichloromethane (20 mL), filtered washing with dichloromethane (2×5 mL). The filtrate was concentrated under reduced pressure to yield the title compound as a light yellow oil (876 mg, 46%).

### (2S)-2-Amino-2-cyclopentylethanol

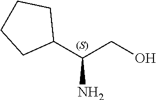

A solution of tert-butyl N-[(1 S)-1-cyclopentyl-2-hydroxyethyl]carbamate (830 mg, 3.62 mmol, 1.0 eq.) and hydrochloric acid in 1,4-dioxane (1.10 mL, 36.2 mmol, 10 eq.) in dichloromethane (10 mL) was stirred for 2 hours at room temperature. The resulting mixture was concentrated under reduced pressure to yield the title compound as a white solid as a white solid (432 mg, 92%).

### 2-Cyclopentyl-2-{[2-(methylsulfanyl)-5-[2-(triisopropylsilyl)ethynyl]pyrido[2,3-d]pyrimidin-7-yl]amino}ethanol

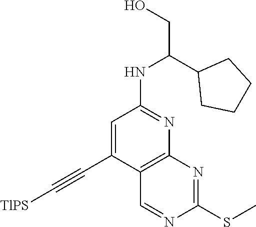

A solution of 7-chloro-2-(methylsulfanyl)-5-[2-(triisopropylsilyl)ethynyl]pyrido[2,3-d]pyrimidine (800 mg, 2.04 mmol, 1.0 eq.), 2-amino-2-cyclopentylethanol (316 mg, 2.45 mmol, 1.2 eq.), XantPhos (118 mg, 0.204 mmol, 0.1 eq.), tris(dibenzyldeneacetone)dipalladium (0) (93.4 mg, 0.102 mmol, 0.05 eq.) and potassium carbonate (564 mg, 4.08 mmol, 2.0 eq.) in dioxane (10 mL) was stirred for 1 hour at 100° C. The mixture was allowed to cool down to room temperature then concentrated under reduced pressure. The residue was purified by flash column chromatography eluting with ethyl acetate (20%) in 40-60 petroleum ether to afford the title compound as a reddish brown oil (462 mg, 46%).

### 4-Cyclopentyl-3-[2-(methylsulfanyl)-5-[2-(triisopropylsilyl)ethynyl]pyrido[2,3-d]pyrimidin-7-yl]-1,3-oxazolidin-2-one

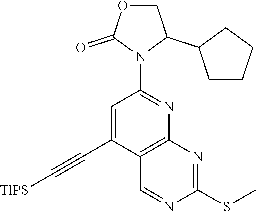

A solution of 2-cyclopentyl-2-{[2-(methylsulfanyl)-5-[2-(triisopropylsilyl)ethynyl]pyrido[2,3-d]pyrimidin-7-yl]amino}ethanol (430 mg, 0.887 mmol, 1.0 eq.), CDI (215 mg, 1.33 mmol, 1.5 eq.) and diisopropylethylamine (229 mg, 1.77 mmol, 2.0 eq.) in DMF (8.0 mL) was stirred for 3 hours at 100° C. The resulting mixture was diluted with water (30 mL) and extracted with ethyl acetate (3×10 mL). The combined organic layers were concentrated under reduced pressure. The residue was purified by flash column chromatography eluting with ethyl acetate (15%) in 40-60 petroleum ether to afford the title compound as a reddish brown solid (387 mg, 85%).

### 4-Cyclopentyl-3-[2-methanesulfonyl-5-[2-(triisopropylsilyl)ethynyl]pyrido[2,3-d]pyrimidin-7-yl]-1.3-oxazolidin-2-one

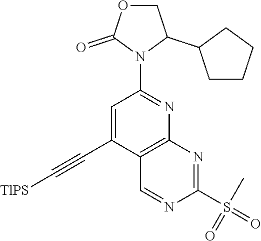

General Procedure 2 was applied to 4-cyclopentyl-3-[2-(methylsulfanyl)-5-[2-(triisopropylsilyl)ethynyl]pyrido[2,3-d]pyrimidin-7-yl]-1,3-oxazolidin-2-one (380 mg, 0.744 mmol) and m-CPBA (128 mg, 0.744 mmol) in dichloromethane (5.0 mL) to yield the title compound as a yellow solid (380 mg, 94%).

Tert-butyl 4-(4-{[7-(4-cyclopentyl-2-oxo-1,3-oxazolidin-3-yl)-5-[2-(triisopropylsilyl)ethynyl]pyrido[2,3-d]pyrimidin-2-yl]amino}phenyl)piperazine-1-carboxylate

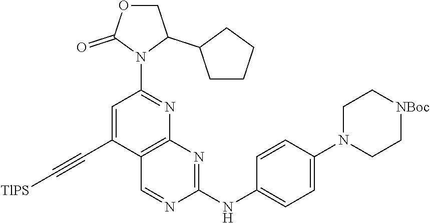

General Procedure 3 was applied to 4-cyclopentyl-3-{2-methanesulfonyl-5-[2-(triisopropylsilyl)ethynyl]pyrido[2,3-d]pyrimidin-7-yl}-1,3-oxazolidin-2-one (380 mg, 0.700 mmol), tert-butyl 4-(4-aminophenyl)piperazine-1-carboxylate (252 mg, 0.910 mmol) and trifluoroacetic acid (0.100 mL, 1.40 mmol) in 2-methyl-2-butanol (2.0 mL), stirred for 1 hour at 100° C. The residue was purified by reverse phase flash column chromatography eluting with acetonitrile (10-60%) in water (0.1% formic acid), to yield the title compound as a yellow solid (220 mg, 42%).

### 4-Cyclopentyl-3-(2-{[4-(piperazin-1-yl)phenyl]amino}-5-[2-(triisopropylsilyl)ethynyl]pyrido[2,3-d]pyrimidin-7-yl)-1,3-oxazolidin-2-one

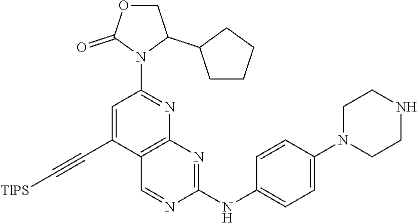

A solution of tert-butyl 4-(4-{[7-(4-cyclopentyl-2-oxo-1,3-oxazolidin-3-yl)-5-[2-(triisopropylsilyl) ethynyl]pyrido[2,3-d]pyrimidin-2-yl]amino}phenyl)piperazine-1-carboxylate (220 mg, 0.297 mmol, 1.0 eq.) and hydrochloric acid in 1,4-dioxane (90.0 μL, 2.97 mmol, 10 eq.) in dichloromethane (5.0 mL) was stirred for 3 hours at room temperature. The resulting mixture was concentrated under reduced pressure to yield the title compound as a yellow solid (132 mg, 69%).

### 4-Cyclopentyl-3-(5-ethynyl-2-{[4-(piperazin-1-yl)phenyl]amino}pyrido[2,3-d]pyrimidin-7-y0)-1,3-oxazolidin-2-one

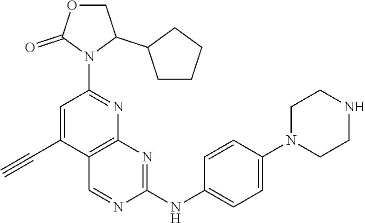 [SMILES: C#Cc1cc(N2C(=O)OCC2C2CCCC2)nc2nc(Nc3ccc(N4CCNCC4)cc3)ncc12]

General Procedure 1 was applied to 4-cyclopentyl-3-(2-{[4-(piperazin-1-yl)phenyl]amino}-5-[2-(triisopropylsilyl)ethynyl]pyrido[2,3-d]pyrimidin-7-yl)-1,3-oxazolidin-2-one (100 mg, 0.156 mmol) and potassium fluoride (90.8 mg, 1.56 mmol) in DMF (2.0 mL), THE (2.0 mL) and water (0.5 mL), stirred for 1 hour at 60° C. The residue was purified by reverse phase flash column chromatography eluting with acetonitrile (10-50%) in water (0.1% formic acid), to yield the title compound as a reddish brown solid (5.60 mg, 7%).

(ES, m/z) [M+H]⁺=484.20

¹H-NMR (300 MHz, DMSO-d₆) δ 10.13 (s, 1H), 9.24 (s, 1H), 8.74 (s, 2H), 7.77 (s, 2H), 7.01 (d, J=9.1 Hz, 2H), 5.13 (s, 2H), 4.56 (t, J=8.5 Hz, 1H), 4.36 (dd, J=8.8, 2.8 Hz, 1H), 3.30 (s, 8H), 2.71 (s, 1H), 1.71-1.41 (m, 6H), 1.26 (d, J=14.2 Hz, 2H).

### Example 116

### 2-((tert-Butoxycarbonyl)amino)-3-methoxypropanoic (isopropyl carbonic) anhydride

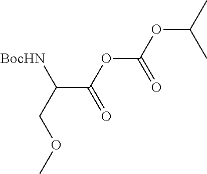 [SMILES: COCC(NC(=O)OC(C)(C)C)C(=O)OC(=O)OC(C)C]

To a stirred mixture of 2-[(tert-butoxycarbonyl)amino]-3-methoxypropanoic acid (1.00 g, 4.56 mmol, 1.0 eq.) and triethylamine (550 mg, 5.47 mmol, 1.2 eq.) in THF (5.0 mL) was added isopropyl chloroformate (610 mg, 5.02 mmol, 1.1 eq.) in portions at 0° C. The resulting mixture was stirred for 30 minutes at 0° C. then filtered washing with THF (2×5 mL). The resulting THF solution was used in the next step directly without further purification.

### tert-butyl N-(1-hydroxy-3-methoxypropan-2-yl)carbamate

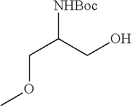

To a stirred solution of sodium borohydride (520 mg, 13.8 mmol, 3.0 eq.) in THF (10 mL) was added 1-isopropoxyethenyl 2-[(tert-butoxycarbonyl)amino]-3-methoxypropanoate (1.40 g, 4.61 mmol, 1.0 eq.) in THF (10 mL) dropwise at 0° C. The resulting mixture was stirred for 2 hours at 0° C. The mixture was acidified to pH 4 with aqueous hydrochloric acid solution. The resulting mixture was diluted with water (20 mL) and extracted with ethyl acetate (3×10 mL). The combined organic layers were washed with saturated aqueous sodium hydrogen carbonate, dried (Na₂SO₄) and concentrated under reduced pressure. The residue was purified by reverse phase flash column chromatography eluting with acetonitrile (0-50%) in water (0.1% ammonium carbonate), to yield the title compound as a colourless solid (500 mg, 52.78%).

¹H-NMR (300 MHz, DMSO-d₆) δ 6.48 (d, J=8.3 Hz, 1H), 4.62 (s, 1H), 3.53 (dq, J=11.6, 6.0 Hz, 1H), 3.36-3.25 (m, 4H), 3.22 (s, 3H), 1.38 (s, 9H).

### 2-Amino-3-methoxypropan-1-ol hydrochloride

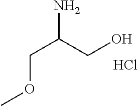

A solution of tert-butyl N-(1-hydroxy-3-methoxypropan-2-yl)carbamate (500 mg, 2.43 mmol, 1.0 eq.) and hydrochloric acid (177 mg, 4.87 mmol, 2.0 eq.) in dioxane (1.0 mL) was stirred for 1 hour at room temperature. The resulting mixture was concentrated under reduced pressure to yield the title compound as a light yellow oil (330 mg, 95%).

### 3-Methoxy-2-{[2-(methylsulfanyl)-5-[2-(triisopropylsilyl)ethynyl]pyrido[2,3-d]pyrimidin-7-yl]amino}propan-1-ol

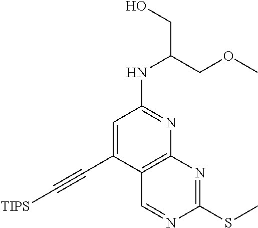

To a solution of 7-chloro-2-(methylsulfanyl)-5-[2-(triisopropylsilyl)ethynyl]pyrido[2,3-d]pyrimidine (1.00 g, 2.55 mmol, 1.0 eq.) and 2-amino-3-methoxypropan-1-ol hydrochloride (404 mg, 2.85 mmol, 1.1 eq.) in dioxane (6.0 mL) were added cesium carbonate (1.66 g, 5.10 mmol, 2.0 eq.), tris(dibenzyldeneacetone)dipalladium (0) (120 mg, 0.128 mmol, 0.05 eq.) and XantPhos (150 mg, 0.255 mmol, 0.10 eq.). The reaction was stirred for 2 hours at 100° C. The reaction was cooled to room temperature then filtered, washing with dichloromethane (3×10 mL). The filtrate was concentrated under reduced pressure. The residue was purified by flash column chromatography eluting with ethyl acetate (10%) in 40-60 petroleum ether to yield the title compound as a light yellow solid (570 mg, 48%).

¹H-NMR (300 MHz, DMSO-d₆) δ 8.84 (s, 1H), 7.99 (d, J=8.2 Hz, 1H), 7.06 (s, 1H), 4.89 (s, 1H), 4.39 (s, 1H), 3.57-3.45 (m, 4H), 3.28 (s, 3H), 2.55 (s, 3H), 1.12 (d, J=4.9 Hz, 21H)

### 4-(Methoxymethyl)-3-[2-(methylsulfanyl)-5-[2-(triisopropylsilyl)ethynyl]pyrido[2,3-d]pyrimidin-7-yl]-1,3-oxazolidin-2-one

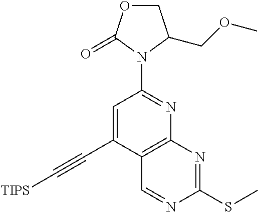

A solution of 3-methoxy-2-{[2-(methylsulfanyl)-5-[2-(triisopropylsilyl)ethynyl]pyrido[2,3-d]pyrimidin-7-yl]amino}propan-1-ol (570 mg, 1.24 mmol, 1.0 eq.), CDI (1.20 g, 7.42 mmol, 6.0 eq.) and diisopropylethylamine (479 mg, 3.71 mmol, 3.0 eq.) in DMF (2.0 mL) was stirred for 2 hours at 80° C. The mixture was allowed to cool down to room temperature then diluted with water (20 mL) and extracted with dichloromethane (3×30 mL). The combined organic layers were washed with brine (2×20 mL), dried (Na₂SO₄) and concentrated under reduced pressure. The residue was purified by flash column chromatography eluting with ethyl acetate (5%) in 40-60 petroleum ether to yield the title compound as a yellow solid (457 mg, 76%).

¹H-NMR (300 MHz, Chloroform-d): δ 9.41 (s, 1H), 8.60 (s, 1H), 5.25 (dtd, J=6.9, 4.4, 2.8 Hz, 1H), 4.60-4.45 (m, 2H), 3.92 (dd, J=9.9, 4.6 Hz, 1H), 3.70 (dd, J=9.9, 2.6 Hz, 1H), 3.35 (s, 3H), 2.71 (s, 3H), 1.30-1.10 (m, 21H).

### 3-{2-Methanesulfonyl-5-[2-(triisopropylsilyl)ethynyl]pyrido[2,3-d]pyrimidin-7-yl}-4-(methoxymethyl)-1,3-oxazolidin-2-one

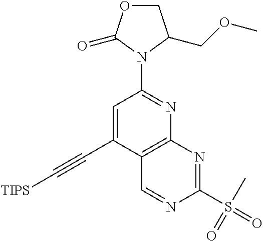

General Procedure 2 was applied to 4-(methoxymethyl)-3-[2-(methylsulfanyl)-5-[2-(triisopropylsilyl)ethynyl]pyrido[2,3-d]pyrimidin-7-yl]-1,3-oxazolidin-2-one (440 mg, 0.904 mmol) and m-CPBA (390 mg, 2.26 mmol) in dichloromethane (3.0 mL) to yield the title compound as a yellow solid (400 mg, 85%).

### 4-(Methoxymethyl)-3-(2-{[4-(4-methylpiperazin-1-yl)phenyl]amino}-5-[2-(triisopropylsilyl)ethynyl]pyrido[2,3-d]pyrimidin-7-yl)-1,3-oxazolidin-2-one

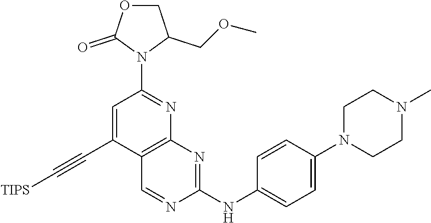

General Procedure 3 was applied to 3-{2-methanesulfonyl-5-[2-(triisopropylsilyl)ethynyl]pyrido[2,3-d]pyrimidin-7-yl}-4-(methoxymethyl)-1,3-oxazolidin-2-one (400 mg, 0.771 mmol), trifluoroacetic acid (176 mg, 1.54 mmol) and 4-(4-methylpiperazin-1-yl)aniline (192 mg, 1.00 mmol) in butan-2-ol (2.0 mL), stirred for 1 hour at 100° C. The residue was purified by Prep-HPLC, eluting with acetonitrile (20-60%) in water (0.05% trifluoroacetic acid), to yield the title compound as a reddish brown solid (252 mg, 52%).

¹H-NMR (300 MHz, Chloroform-d) δ 9.32 (s, 1H), 8.37 (s, 1H), 7.54 (d, J=7.1 Hz, 3H), 7.02-6.90 (m, 2H), 5.16 (dq, J=6.8, 3.2, 2.7 Hz, 1H), 4.56-4.41 (m, 2H), 3.89 (dd, J=9.9, 4.6 Hz, 1H), 3.67 (dd, J=9.9, 2.7 Hz, 1H), 3.34 (s, 3H), 3.21 (dd, J=6.3, 3.7 Hz, 4H), 2.61 (dd, J=6.2, 3.8 Hz, 4H), 2.37 (s, 3H), 1.22-1.13 (m, 21H).

### 3-(5-Ethynyl-2-{[4-(4-methylpiperazin-1-yl)phenyl]amino}pyrido[2,3-d]pyrimidin-7-yl)-4-(methoxymethyl)-1,3-oxazolidin-2-one

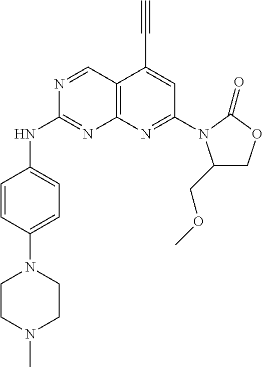 [SMILES: C#Cc1cc(N2C(=O)OCC2COC)nc2nc(Nc3ccc(N4CCN(C)CC4)cc3)ncc12]

General Procedure 1 was applied to 4-(methoxymethyl)-3-(2-{[4-(4-methylpiperazin-1-yl)phenyl]amino}-5-[2-(triisopropylsilyl)ethynyl]pyrido[2,3-d]pyrimidin-7-yl)-1,3-oxazolidin-2-one (242 mg, 0.384 mmol) and potassium fluoride (223 mg, 3.84 mmol) in THF (3.0 mL), DMF (1.0 mL) and water (4.0 mL), stirred for 1 hour at 60° C. The residue was purified by Prep-HPLC, eluting with methanol (20-80%) in water (0.1% ammonium acetate), to yield the title compound as an orange solid (25.0 mg, 14%).

(ES, m/z) [M+H]⁺=474.15

¹H-NMR (400 MHz, DMSO-d₆) δ 10.07 (s, 1H), 9.25 (s, 1H), 8.21 (s, 1H), 7.75 (s, 2H), 6.98 (d, J=8.6 Hz, 2H), 5.15 (s, 1H), 5.10 (s, 1H), 4.60 (t, J=8.5 Hz, 1H), 4.44 (dd, J=8.4, 3.1 Hz, 1H), 3.93 (dd, J=9.9, 4.0 Hz, 1H), 3.68 (dd, J=9.9, 2.4 Hz, 1H), 3.31 (s, 3H), 3.14 (t, J=5.0 Hz, 4H), 2.50 (t, J=4.9 Hz, 4H), 2.27 (s, 3H).

### Example 117

### 4-Nitro-2,3-dihydro-1H-isoindole

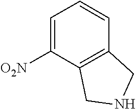 [SMILES: O=[N+]([O-])c1cccc2c1CNC2]

A solution of 3-nitrophthalimide (5.00 g, 26.0 mmol, 1.0 eq.) and sodium borohydride (9.84 g, 260 mmol, 10 eq.) in THF (50 mL) was stirred for 30 min at 0° C. then boron trifluoride etherate (32.0 mL, 10 eq.) was added dropwise over 10 minutes at 0 C. The resulting mixture was stirred overnight at 70C. The mixture was allowed to cool down to room temperature then quenched by the addition of water/ice (100 mL) at 0° C. The mixture was acidified to pH 2 with hydrogen chloride in 1,4-dioxane (4M). The aqueous layer was extracted with ethyl acetate (200 mL). The aqueous layer was basified to pH 9 with sodium hydroxide. The aqueous layer was extracted with ethyl acetate (5×100 mL). The combined organic layers were concentrated under reduced pressure. The residue was purified by flash column chromatography, eluting with ethyl acetate (50%) in 40-60 petroleum ether to afford the title compound as a black solid (2.10 g, 49%).

### 2-Methyl-4-nitro-1,3-dihydroisoindole

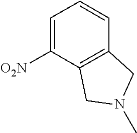

A solution of 4-nitro-2,3-dihydro-1H-isoindole (2.00 g, 12.2 mmol, 1.0 eq.), formaldehyde (3.66 g, 122 mmol, 10 eq.) in water (10 mL) and acetic acid (1.05 mL, 18.3 mmol, 1.5 eq.) in methanol (20 mL) was stirred for 20 minutes at 0° C. To the above mixture was added sodium borohydride (1.38 g, 36.5 mmol, 3.0 eq.) in portions over 10 minutes at 0° C. The resulting mixture was stirred for 30 minutes at 0° C. then diluted with water (100 mL) and extracted with ethyl acetate (2×100 mL). The combined organic layers were concentrated under reduced pressure to yield the title compound as a black solid (1.92 g, 88%).

### 2-Methyl-1,3-dihydroisoindol-4-amine

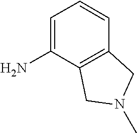

To a stirred solution of 2-methyl-4-nitro-1,3-dihydroisoindole (1.50 g, 8.42 mmol, 1.0 eq.) in dichloromethane (20 mL) was added palladium on carbon (10%, 270 mg). The mixture was hydrogenated at room temperature overnight under hydrogen atmosphere, then filtered through a Celite pad and concentrated under reduced pressure to yield the title compound as a black oil (1.15 g, 92%).

### 3-Cyclopentyl-1-{2-[(2-methyl-1,3-dihydroisoindol-4-yl)amino]-5-[2-(triisopropylsilyl)ethynyl]pyrido[2,3-d]pyrimidin-7-yl}urea

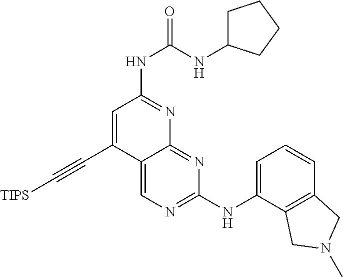

General Procedure 3 was applied to 3-cyclopentyl-1-{2-methanesulfinyl-5-[2-(triisopropylsilyl)ethynyl]pyrido[2,3-d]pyrimidin-7-yl}urea (800 mg, 1.60 mmol), 2-methyl-1,3-dihydroisoindol-4-amine (285 mg, 1.92 mmol) and trifluoroacetic acid (0.240 mL, 3.20 mmol) in 2-methyl-2-butanol (10 mL), stirring overnight at 100° C. The residue was purified by reverse phase flash column chromatography eluting with acetonitrile (10-50%) in water (0.1% formic acid) to yield the title compound as a brown solid (195 mg, 21%).

### 3-Cyclopentyl-1-{5-ethynyl-2-[(2-methyl-1,3-dihydroisoindol-4-yl)amino]pyrido[2,3-d]pyrimidin-7-yl}urea

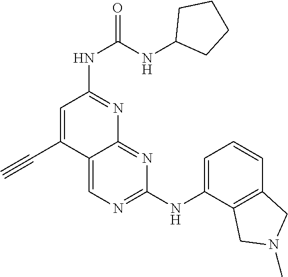 [SMILES: C#Cc1cc(NC(=O)NC2CCCC2)nc2nc(Nc3cccc4c3CN(C)C4)ncc12]

General Procedure 1 was applied to 3-cyclopentyl-1-{2-[(2-methyl-1,3-dihydroisoindol-4-yl)amino]-5-[2-(triisopropylsilyl)ethynyl]pyrido[2,3-d]pyrimidin-7-yl}urea (130 mg, 0.223 mmol) and potassium fluoride (64.7 mg, 1.12 mmol) in DMF (2.0 mL), stirring for 1 hour at 60° C. The residue was purified by reverse phase flash column chromatography eluting with acetonitrile (10-70%) in water (0.1% formic acid), to yield the title compound as a light yellow solid (55.5 mg, 58%).

(ES, m/z) [M+H]⁺=428.05.

¹H-NMR (300 MHz, DMSO-d₆) δ 10.01 (s, 1H), 9.64 (s, 1H), 9.41 (s, 1H), 9.08 (s, 1H), 7.62 (d, J=8.0 Hz, 1H), 7.24-7.13 (m, 2H), 7.02 (d, J=7.4 Hz, 1H), 5.06 (s, 1H), 4.14-4.04 (m, 1H), 2.45 (s, 3H), 1.88 (dt, J=12.5, 6.1 Hz, 2H), 1.75-1.57 (m, 4H), 1.48 (dd, J=11.7, 6.0 Hz, 2H).

### Example 118

### tert-Butyl 3-{[(4-methylbenzenesulfonyl)oxy]methyl}piperidine-1-carboxylate

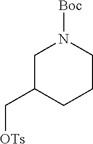 [SMILES: Cc1ccc(S(=O)(=O)OCC2CCCN(C(=O)OC(C)(C)C)C2)cc1]

To a stirred solution of tert-butyl 3-(hydroxymethyl)piperidine-1-carboxylate (6.00 g, 27.9 mmol, 1.0 eq.) in dichloromethane (30 mL) was added triethylamine (7.05 g, 69.7 mmol, 2.5 eq.) and tosyl chloride (6.38 g, 33.4 mmol, 1.2 eq.) in dichloromethane (30 mL) dropwise at 0° C. The resulting mixture was stirred for 4 hours at room temperature then quenched by the addition of saturated aqueous sodium hydrogen carbonate (100 mL). The resulting mixture was extracted with dichloromethane (3×50 mL). The combined organic layers were washed with brine (200 mL), dried (Na₂SO₄) and concentrated under reduced pressure. The residue was purified by flash column chromatography, eluting with ethyl acetate (15%) in 40-60 petroleum ether to afford the title compound as a colourless oil (9.50 g, 92%).

### tert-Butyl 3-[(methylsulfanyl)methyl]piperidine-1-carboxylate

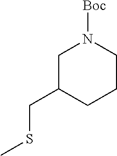 [SMILES: CSCC1CCCN(C(=O)OC(C)(C)C)C1]

To a stirred solution of tert-butyl 3-{[(4-methylbenzenesulfonyl)oxy]methyl}piperidine-1-carboxylate (4.70 g, 12.7 mmol, 1.0 eq.) in methanol (50 mL) was added sodiummethanethiolate (3.57 g, 50.9 mmol, 4.0 eq.). The resulting mixture was stirred overnight at 60° C. then poured into water and extracted with dichloromethane (3×50 mL). The combined organic layers were washed with brine (100 mL), dried (Na₂SO₄) then concentrated under reduced pressure to yield the title compound as a colourless oil (3.00 g, 96%). The crude product was used in the next step directly without further purification.

### tert-Butyl 3-(methanesulfonylmethyl)piperidine-1-carboxylate

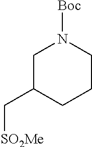 [SMILES: CC(C)(C)OC(=O)N1CCCC(CS(C)(=O)=O)C1]

To a stirred solution of tert-butyl 3-[(methylsulfanyl)methyl]piperidine-1-carboxylate (3.20 g, 13.0 mmol, 1.0 eq.) in methanol (15 mL) and water (15 mL) were added oxone (8.77 g, 52.2 mmol, 4.0 eq.) in portions at 0° C. The resulting mixture was stirred for 3 hours at room temperature then diluted with water (50 mL) and extracted with dichloromethane (3×50 mL). The combined organic layers were washed with brine (100 mL), dried (Na₂SO₄) and concentrated under reduced pressure to yield the title compound as a colourless oil (1.90 g, 52%). The crude product was used in the next step directly without further purification.

### 3-(Methanesulfonylmethyl)piperidine hydrochloride

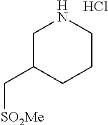 [SMILES: Cl ; CS(=O)(=O)CC1CCCNC1]

A solution of tert-butyl 3-(methanesulfonylmethyl)piperidine-1-carboxylate (1.90 g, 6.85 mmol, 1.0 eq.) and 4 M hydrochloric acid in dioxane (20 mL) was stirred for 2 hours at room temperature then concentrated under reduced pressure to yield the title compound as a white solid (1.44 g, 98%). The crude product was used in the next step directly without further purification.

### 3-(Methanesulfonylmethyl)-1-(2-nitrophenyl)piperidine

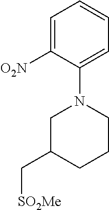 [SMILES: CS(=O)(=O)CC1CCCN(c2ccccc2[N+](=O)[O-])C1]

To a stirred solution of 3-(methanesulfonylmethyl)piperidine hydrochloride (1.44 g, 6.74 mmol, 1.0 eq.) and cesium carbonate (6.59 g, 20.2 mmol, 3.0 eq.) in acetonitrile (15 mL) was added O-fluoronitrobenzene (951 mg, 6.74 mmol, 1.0 eq.) at room temperature. The resulting mixture was stirred for 2 hours at 80° C. then diluted with water (30 mL) and extracted with dichloromethane (3×50 mL). The combined organic layers were washed with brine (100 mL), dried (Na₂SO₄) and concentrated under reduced pressure. The residue was purified by flash column chromatography eluting with ethyl acetate (15%) in 40-60 petroleum ether to afford the title compound as a white solid (1.73 g, 86%).

### 2-[3-(Methanesulfonylmethyl)piperidin-1-yl]aniline

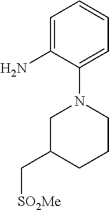 [SMILES: CS(=O)(=O)CC1CCCN(c2ccccc2N)C1]

General Procedure 5 was applied to 3-(methanesulfonylmethyl)-1-(2-nitrophenyl)piperidine (1.73 g, 5.80 mmol) and palladium on carbon(20% wt, 350 mg) in methanol (20 mL) to yield the title compound as a white solid (1.36 g, 87%). The crude product was used in the next step directly without further purification.

### 3-Cyclopentyl-1-[2-({2-[3-(methanesulfonylmethyl)piperidin-1-yl]phenyl}amino)-5-[2-(triisopropylsilyl)ethynyl]pyrido[2,3-d]pyrimidin-7-yl]urea

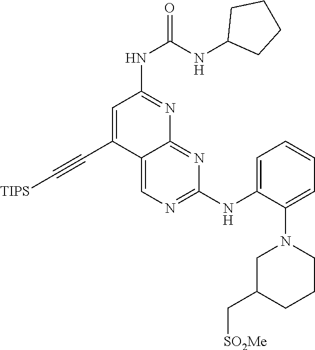

General Procedure 3 was applied to 3-cyclopentyl-1-{2-methanesulfinyl-5-[2-(triisopropylsilyl)ethynyl]pyrido[2,3-d]pyrimidin-7-yl}urea (150 mg, 0.300 mmol), 2-[3-(methanesulfonylmethyl)piperidin-1-yl]aniline (121 mg, 0.450 mmol) and trifluoroacetic acid (68.4 mg, 0.600 mmol) in butan-2-ol (4.0 mL), stirring for 4 hours at 100° C. The residue was purified by flash column chromatography eluting with ethyl acetate (10%) in dichloromethane to afford the title compound as a yellow solid (170 mg, 80%).

### 3-Cyclopentyl-1-[5-ethynyl-2-({2-[3-(methanesulfonylmethyl)piperidin-1-yl]phenyl}amino)pyrido[2,3-d]pyrimidin-7-yl]urea

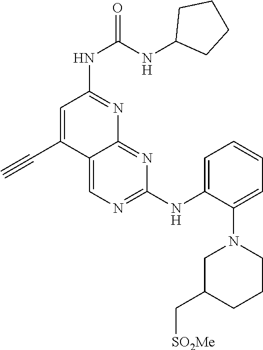 [SMILES: C#Cc1cc(NC(=O)NC2CCCC2)nc2nc(Nc3ccccc3N3CCCC(CS(C)(=O)=O)C3)ncc12]

General Procedure 1 was applied to 3-cyclopentyl-1-[2-({2-[3-(methanesulfonylmethyl)piperidin-1-yl]phenyl}amino)-5-[2-(triisopropylsilyl)ethynyl]pyrido[2,3-d]pyrimidin-7-yl]urea (140 mg, 0.200 mmol) and potassium fluoride (115 mg, 2.00 mmol) in DMF (2.0 mL), THF (2.0 mL) and water (40 uL), stirring for 4 hours at 60C. The crude material was purified by reverse phase flash column chromatography eluting with acetonitrile (10-100%) in water (0.1% formic acid), to yield the title compound as a yellow solid (31.6 mg, 29%).

(ES, m/z) [M+H]⁺=548.20

¹H-NMR (300 MHz, DMSO-d₆) δ 10.09 (s, 1H), 9.61-9.43 (m, 1H), 9.12 (s, 1H), 8.75 (s, 1H), 8.73-8.68 (m, 1H), 7.29-7.20 (m, 2H), 7.17-7.02 (m, 2H), 5.08 (s, 1H), 4.23-4.08 (m, 1H), 3.26-3.10 (m, 3H), 3.00 (s, 3H), 2.94-2.82 (m, 1H), 2.77-2.57 (m, 2H), 2.44-2.32 (m, 1H), 2.04-1.65 (m, 9H), 1.63-1.51 (m, 2H), 1.41-1.28 (m, 1H).

### Example 119

### (1s,3s)-3-[(tert-Butoxycarbonyl)(methyl)amino]cyclobutane-1-carboxylic acid

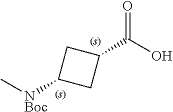

To a solution of (1s,3s)-3-[(tert-butoxycarbonyl)amino]cyclobutane-1-carboxylic acid (24.0 g, 111 mmol, 1.0 eq.) in THF was added sodium hydride (60% in oil, 8.00 g) at 0° C. The mixture was stirred for 15 minutes then methyl iodide (47.5 g, 334 mmol, 3.0 eq.) was added and the mixture warmed to room temperature and stirred for 16 hours. The reaction was quenched with water/ice at 0° C. then concentrated under reduced pressure. The resulting residue was washed with 2×200 mL of MTBE. The mixture was acidified to pH 4 with hydrochloric acid (2 M) then extracted with dichloromethane (2×300 mL). The combined organic layers were washed with brine (2×100 mL), dried (Na₂SO₄) and concentrated under reduced pressure to yield the title compound as a white solid (24.0 g, 94%).

### tert-Butyl N-methyl-N-[(1s,3s)-3-(hydroxymethyl)cyclobutyl]carbamate

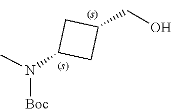

To a stirred solution of (1s,3s)-3-[(tert-butoxycarbonyl)(methyl)amino]cyclobutane-1-carboxylic acid (24.0 g, 105 mmol, 1.0 eq.) in THF (240 mL) was added borane-THF complex (20.0 mL, 209 mmol, 2.0 eq.) dropwise at 0° C. The resulting mixture was stirred overnight at room temperature under hydrogen atmosphere. The reaction was quenched with water/ice at 0° C. then extracted with ethyl acetate (2×300 mL). The combined organic layers were washed with brine (2×200 mL), dried (Na₂SO₄) and concentrated under reduced pressure to yield the title compound as a colourless oil (20.0 g, 89%).

### tert-Butyl N-methyl-N-[(1s,3s)-3-([(4-methylbenzenesulfonyl)oxy]methylcyclobutyl]carbamate

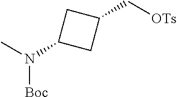 [SMILES: Cc1ccc(S(=O)(=O)OC[C@H]2C[C@@H](N(C)C(=O)OC(C)(C)C)C2)cc1]

To a stirred mixture of tert-butyl N-methyl-N-[(1s,3s)-3-(hydroxymethyl)cyclobutyl]carbamate (20.0 g, 93 mmol, 1.0 eq.) and triethylamine (32.3 mL, 232 mmol, 2.5 eq.) in dichloromethane (200 mL) was added tosyl chloride (19.5 g, 102 mmol, 1.1 eq.) in portions at room temperature. The resulting mixture was stirred overnight at room temperature. The residue was purified by reverse phase flash column chromatography eluting with acetonitrile (30-80%) in water (0.1% trifluoroacetic acid) to yield the title compound as a white solid (10.0 g, 29%).

### tert-Butyl N-methyl-N-[(1s,3s)-3-[(methylsulfanyl)methyl]cyclobutyl]carbamate

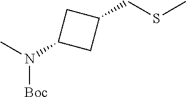

A mixture of tert-butyl N-methyl-N-[(1s,3s)-3-([(4-methylbenzenesulfonyl)oxy]methylcyclobutyl]carbamate (10.0 g, 27.0 mmol, 1.0 eq.) and sodiummethanethiolate (7.59 g, 108 mmol, 4.0 eq.) in DMF (100 mL) was stirred overnight at room temperature. The resulting mixture was extracted with ethyl acetate (2×200 mL). The combined organic layers were washed with brine (2×100 mL), dried (Na₂SO₄) and concentrated under reduced pressure to yield the title compound as a colourless solid (8.00 g, crude).

### tert-butyl N-methyl-N-[(1s,3s)-3-(methanesulfonylmethyl)cyclobutyl]carbamate

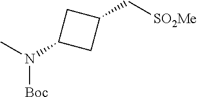 [SMILES: CN(C(=O)OC(C)(C)C)[C@H]1C[C@@H](CS(C)(=O)=O)C1]

A mixture of tert-butyl N-methyl-N-[(1s,3s)-3-[(methylsulfanyl)methyl]cyclobutyl]carbamate (2.00 g, 8.15 mmol, 1.0 eq.) and oxone (5.48 g, 32.6 mmol, 4.0 eq.) in methanol (10 mL) and water (10 mL) was stirred for 3 hours at room temperature. The resulting mixture was extracted with ethyl acetate (2×100 mL). The combined organic layers were washed with brine (2×100 mL), dried (Na₂SO₄) and concentrated under reduced pressure to yield the title compound as a colourless solid (1.50 g, 66.%).

### (1s,3s)-3-(Methanesulfonylmethyl)-N-methylcyclobutan-1-amine

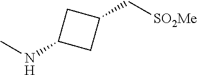

A solution of tert-butyl N-methyl-N-[(1s,3s)-3-(methanesulfonylmethyl)cyclobutyl]carbamate (1.50 g, 5.41 mmol, 1.0 eq.) in hydrogen chloride in 1,4-dioxane (15 mL) was stirred for 3 hours at room temperature then concentrated under reduced pressure to yield the title compound as a colourless solid (1.00 g, crude).

### N-Methyl-3-nitro-N-[(1s,3s)-3-(methanesulfonylmethyl)cyclobutyl]aniline

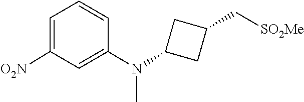 [SMILES: CN(c1cccc([N+](=O)[O-])c1)[C@H]1C[C@@H](CS(C)(=O)=O)C1]

To a solution of (1s,3s)-3-(methanesulfonylmethyl)-N-methylcyclobutan-1-amine (1.00 g, 5.64 mmol, 1.0 eq.) and 3-bromo-1-nitrobenzene (1.25 g, 6.21 mmol, 1.1 eq.) in dioxane (10 mL) was added cesium carbonate (3.68 g, 11.3 mmol, 2.0 eq.), RuPhos (260 mg, 0.564 mmol, 0.1 eq.) and Pd₂(dba)₃ (260 mg, 0.282 mmol, 0.05 eq.). After stirring overnight at 80° C., the reaction mixture was concentrated under reduced pressure. The residue was purified by flash column chromatography eluting with ethyl acetate (50%) in 40-60 petroleum ether to afford the title compound as a yellow solid (1.00 g, 59%).

### N¹-Methyl-N¹-[(1s,3s)-3-(methanesulfonylmethyl)cyclobutyl]benzene-1,3-diamine

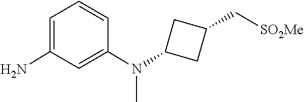 [SMILES: CN(c1cccc(N)c1)[C@H]1C[C@@H](CS(C)(=O)=O)C1]

General Procedure 5 was applied to N-methyl-3-nitro-N-[(1s,3s)-3-(methanesulfonylmethyl)cyclobutyl]aniline (700 mg, 2.35 mmol) and palladium on carbon (140 mg, 1.32 mmol) in methanol (20 mL) to yield the title compound as a yellow solid (600 mg, 95%).

### 3-Cyclopentyl-1-(2-[(3-(methyl[(1 s,3s)-3-(methanesulfonylmethyl)cyclobutyl]aminophenyl)amino]-5-[2-(triisopropylsilyl)ethynyl]pyrido[2,3-d]pyrimidin-7-ylurea

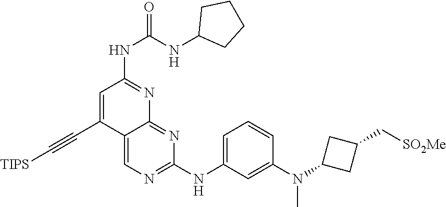

General Procedure 3 was applied to N¹-methyl-N¹-[(1s,3s)-3-(methanesulfonylmethyl)cyclobutyl]benzene-1,3-diamine (120 mg, 0.447 mmol), 3-cyclopentyl-1-(2-methanesulfinyl-5-[2-(triisopropylsilyl)ethynyl]pyrido[2,3-d]pyrimidin-7-ylurea (268 mg, 0.536 mmol) and trifluoroacetic acid (102 mg, 0.894 mmol) in butan-2-ol (2 mL), stirring for 1 hour at 100° C. The crude product was purified by Prep-HPLC eluting with acetonitrile (70-90%) in water(0.1% ammonium formate and 0.05% aqueous ammonia), to afford the title compound as a yellow solid (50.0 mg, 16%).

### 3-Cyclopentyl-1-(5-ethynyl-2-[(3-(methyl[(1s,3s)-3-(methanesulfonylmethyl)cyclobutyl]aminophenyl]aminopyrido[2,3-d]pyrimidin-7-ylurea

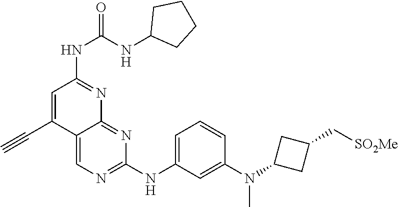 [SMILES: C#Cc1cc(NC(=O)NC2CCCC2)nc2nc(Nc3cccc(N(C)[C@H]4C[C@@H](CS(C)(=O)=O)C4)c3)ncc12]

General Procedure 1 was applied to 3-cyclopentyl-1-(2-[(3-(methyl[(1s,3s)-3-(methanesulfonylmethyl)cyclobutyl]aminophenyl)amino]-5-[2-(triisopropylsilyl)ethynyl]pyrido[2,3-d]pyrimidin-7-ylurea (70.0 mg, 99.0 μmol) and potassium fluoride (57.7 mg, 0.990 mmol) in THF (0.50 mL), DMF (0.50 mL) and water (50 uL), stirring for 2 hour at room temperature. The crude product was purified by Prep-HPLC eluting with acetonitrile (30-55%) in water(0.1% formic acid), to afford the title compound as a yellow solid (6.00 mg, 11%).

(ES, m/z) [M+H]⁺=548.20

¹H-NMR (400 MHz, DMSO-d₆) δ 9.99 (d, J=17.4 Hz, 2H), 9.09 (s, 1H), 7.74 (s, 1H), 7.19 (s, 1H), 7.13-7.06 (m, 2H), 6.49 (d, J=8.6 Hz, 1H), 5.07 (s, 1H), 4.20-4.08 (m, 1H), 3.83 (q, J=7.9 Hz, 1H), 3.31 (s, 2H), 2.93 (s, 3H), 2.77 (s, 3H), 2.57 (d, J=7.9 Hz, 2H), 2.41 (s, 2H), 1.96-1.83 (m, 4H), 1.79 (d, J=4.7 Hz, 2H), 1.68 (d, J=7.8 Hz, 2H), 1.60-1.47 (m, 2H).

### Example 120

### (1s,4s)-4-[(tert-Butoxycarbonyl)(methyl)amino]cyclohexane-1-carboxylic acid

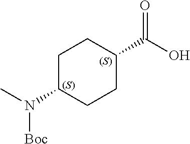

To a stirred solution of (1s,4s)-4-[(tert-butoxycarbonyl)amino]cyclohexane-1-carboxylic acid (23.5 g, 96.6 mmol, 1.0 eq.) in THE was added sodium hydride (11.6 g, 483 mmol, 5.0 eq.) at 0° C. The reaction mixture was stirred for 30 minutes at 0° C. then methyl iodide (18.0 mL, 290 mmol, 3.0 eq.) was added at 0° C. The reaction was stirred for overnight at room temperature then quenched with water/ice (500 mL) and concentrated under reduced pressure. The aqueous layer was extracted with MTBE (200 mL). The aqueous layer was acidified to pH 3 with aqueous hydrochloric acid then extracted with dichloromethane (3×250 mL). The combined organic layers were washed brine (200 mL), dried (Na₂SO₄) and concentrated under reduced pressure to yield the title compound as a white solid (23.4 g, 94%).

### tert-Butyl N-methyl-N-[(1s,4s)-4-(hydroxymethyl)cyclohexyl]carbamate

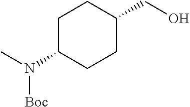 [SMILES: CN(C(=O)OC(C)(C)C)[C@H]1CC[C@@H](CO)CC1]

To a stirred solution of (1s,4s)-4-[(tert-butoxycarbonyl)(methyl)amino]cyclohexane-1-carboxylic acid (23.0 g, 89.4 mmol, 1.0 eq.) in THF (50 mL) was added borane-THF complex (1 M, 207 mL, 2.0 eq.) in portions at 0° C. The resulting mixture was stirred for 2 hours at room temperature then quenched with water/ice (300 mL). The resulting mixture was concentrated under reduced pressure then extracted with dichloromethane (2×300 mL). The combined organic layers were washed with brine (2×100 mL), dried (Na₂SO₄) and concentrated under reduced pressure to yield the title compound as a colourless oil (20.0 g, 92%).

### tert-Butyl N-methyl-N-[(1s,4s)-4-([(4-methylbenzenesulfonyl)oxy]methylcyclohexyl]carbamate

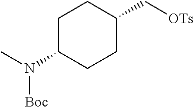 [SMILES: Cc1ccc(S(=O)(=O)OC[C@H]2CC[C@@H](N(C)C(=O)OC(C)(C)C)CC2)cc1]

A solution of tert-butyl N-methyl-N-[(1s,4s)-4-(hydroxymethyl)cyclohexyl]carbamate (20.0 g, 82.2 mmol, 1.0 eq.), tosyl chloride (23.5 g, 123 mmol, 1.5 eq.) and triethylamine (34.3 mL, 246 mmol, 3.0 eq.) in dichloromethane (200 mL) was stirred for 4 hours at room temperature. The residue was purified by reverse phase flash column chromatography eluting with acetonitrile (50-68%) in water (0.1% formic acid), to afford the title compound as a white solid (19.0 g, 58%).

### tert-Butyl N-methyl-N-[(1s,4s)-4-[(methylsulfanyl)methyl]cyclohexyl]carbamate

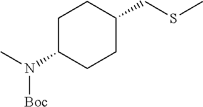

A solution of tert-butyl N-methyl-N-[(1s,4s)-4-([(4-methylbenzenesulfonyl)oxy]methylcyclohexyl]carbamate (10.0 g, 25.2 mmol, 1.0 eq.) and (methylsulfanyl)sodium (5.29 g, 75.5 mmol, 3.0 eq.) in methanol (100 mL) was stirred for overnight at room temperature. The resulting mixture was washed with saturated aqueous sodium hydrogen carbonate (300 mL) and extracted with ethyl acetate (3×150 mL). The combined organic layers were washed with brine (200 mL), dried (Na₂SO₄) and concentrated under reduced pressure to afford the crude title compound as a colourless oil (6.86 g, 100%).

### tert-Butyl N-methyl-N-[(1s,4s)-4-(methanesulfonylmethyl)cyclohexyl]carbamate

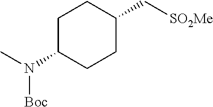 [SMILES: CN(C(=O)OC(C)(C)C)[C@H]1CC[C@@H](CS(C)(=O)=O)CC1]

To a stirred solution of tert-butyl N-methyl-N-[(1s,4s)-4-[(methylsulfanyl)methyl]cyclohexyl]carbamate (5.00 g, 18.3 mmol, 1.0 eq.) in THF (50.0 mL) and water (50.0 mL) was added oxone (15.4 g, 91.4 mmol, 5.0 eq.) at 0° C. The reaction mixture was stirred for 3 hours at room temperature then extracted with ethyl acetate (3×100 mL). The combined organic layers were washed with brine (150 mL), dried (Na₂SO₄) and concentrated under reduced pressure to yield the crude title compound as a colourless oil (4.92 g, 88%).

### (1s,4s)-4-(Methanesulfonylmethyl)-N-methylcyclohexan-1-amine

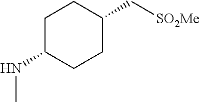

A solution of tert-butyl N-methyl-N-[(1s,4s)-4-(methanesulfonylmethyl)cyclohexyl]carbamate (5.00 g, 16.4 mmol, 1.0 eq.) and hydrogen chloride in methanol (50.0 mL, 1645 mmol, 100 eq.) was stirred for 2 hours at room temperature. The resulting mixture was concentrated under reduced pressure to afford the crude title compound as an off-white solid (3.75 g, 111%).

N-Methyl-3-nitro-N-[(1s,4s)-4-(methanesulfonylmethyl)cyclohexyl]aniline

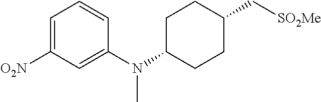 [SMILES: CN(c1cccc([N+](=O)[O-])c1)[C@H]1CC[C@@H](CS(C)(=O)=O)CC1]

A solution of (1s,4s)-4-(methanesulfonylmethyl)-N-methylcyclohexan-1-amine (2.00 g, 9.74 mmol, 1.0 eq.), 3-bromo-1-nitrobenzene (2.16 g, 10.7 mmol, 1.1 eq.), cesium carbonate (6.35 g, 19.5 mmol, 2.0 eq.), Ruphos (455 mg, 0.974 mmol, 0.1 eq.) and Pd₂(dba)₃ (446 mg, 0.487 mmol, 0.05 eq.) in dioxane (22 mL) was stirred overnight at 80° C. The resulting mixture was concentrated under reduced pressure. The residue was purified by flash column chromatography eluting with ethyl acetate (50%) in 40-60 petroleum ether to afford the title compound as a reddish brown solid (1.57 g, 49%).

### N-Methyl-N¹-[(1s,4s)-4-(methanesulfonylmethyl)cyclohexyl]benzene-1,3-diamine

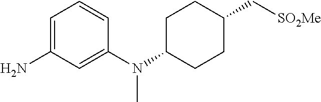 [SMILES: CN(c1cccc(N)c1)[C@H]1CC[C@@H](CS(C)(=O)=O)CC1]

General Procedure 5 was applied to N-methyl-3-nitro-N-[(1s,4s)-4-(methanesulfonylmethyl)cyclohexyl]aniline (200 mg, 0.613 mmol) and palladium on carbon (10%, 70 mg, 0.658 mmol) in THF (20 mL) to yield the crude title compound as a brown oil (232 mg, 127%).

### 3-Cyclopentyl-1-(2-[(3-(methyl[f(1 s,4s)-4-(methanesulfonylmethyl)cyclohexyl]aminophenyl)amino]-5-[2-(triisopropylsilyl)ethynyl]pyrido[2,3-d]pyrimidin-7-yiurea

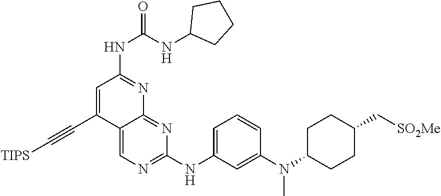

General Procedure 3 was applied to N1-methyl-N1-[(1s,4s)-4-(methanesulfonylmethyl)cyclohexyl]benzene-1,3-diamine (133 mg, 0.450 mmol), 3-cyclopentyl-1-(2-methanesulfinyl-5-[2-(triisopropylsilyl)ethynyl]pyrido[2,3-d]pyrimidin-7-ylurea (150 mg, 0.300 mmol) and trifluoroacetic acid (45.9 μL, 0.600 mmol) in 2-butanol (1.5 mL, 16.351 mmol, 54.48 eq.) stirred for 2 hours at 100° C. The residue was purified by reverse phase flash column chromatography eluting acetonitrile (10-80%) in water (0.1% aqueous ammonia), to afford the title compound as an orange solid (151 mg, 68%).

### 3-Cyclopentyl-1-(5-ethynyl-2-[(3-(methyl[(1s,4s)-4-(methanesulfonylmethyl)cyclohexyl]aminophenyl)amino]pyrido[2,3-d]pyrimidin-7-ylurea

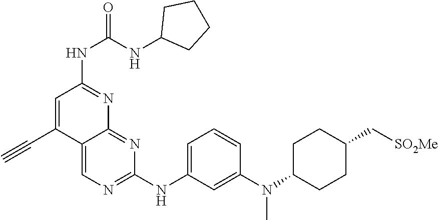 [SMILES: C#Cc1cc(NC(=O)NC2CCCC2)nc2nc(Nc3cccc(N(C)[C@H]4CC[C@@H](CS(C)(=O)=O)CC4)c3)ncc12]

General Procedure 1 was applied to 3-cyclopentyl-1-(2-[(3-(methyl[(1s,4s)-4-(methanesulfonylmethyl)cyclohexyl]aminophenyl)amino]-5-[2-(triisopropylsilyl)ethynyl]pyrido[2,3-d]pyrimidin-7-ylurea (140 mg, 0.191 mmol) and potassium fluoride (111 mg, 1.91 mmol) in THF (1.4 mL), DMF (1.4 mL) and water (140 μL), stirred for 2 hours at 60° C. The residue was purified by reverse phase flash column chromatography eluting with acetonitrile (10-45%) in water (0.1% formic acid) to yield the title compound as a yellow solid (52.1 mg, 47%).

(ES, m/z) [M+H]⁺=576.15

¹H-NMR (400 MHz, DMSO-d₆) δ 10.01 (s, 1H), 9.87 (s, 1H), 9.48 (s, 1H), 9.08 (s, 1H), 7.72 (s, 1H), 7.18 (s, 1H), 7.12-7.00 (m, 2H), 6.53-6.43 (m, 1H), 5.06 (s, 1H), 4.21-4.03 (m, 1H), 3.56 (s, 1H), 3.30-3.28 (s, 2H), 3.00 (d, J=6.5 Hz, 3H), 2.75 (d, J=14.4 Hz, 3H), 2.39 (s, 1H), 2.08-1.40 (m, 15H), 1.27 (d, J=13.0 Hz, 1H).

### Example 121

### (1r,4r)-4-[(tert-Butoxycarbonyl)(methyl)amino]cyclohexane-1-carboxylic acid

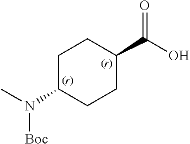

To a stirred solution of (1r,4r)-4-[(tert-butoxycarbonyl)amino]cyclohexane-1-carboxylic acid (20.0 g, 82.2 mmol, 1.0 eq.) in THF (200 mL) was added sodium hydride (9.86 g, 411 mmol, 5.0 eq.) at 0° C. The resulting mixture was stirred for 30 minutes at 0° C. then methyl iodide (15.3 mL, 247 mmol, 3.0 eq.) was added at 0° C. The reaction mixture was stirred overnight at room temperature then quenched with water/ice (200 mL). The resulting mixture was concentrated under reduced pressure then extracted with MTBE (100 mL). The mixture was acidified to pH 3 with aqueous hydrochloric acid and extracted with dichloromethane (3×100 mL). The combined organic layers were washed with brine (150 mL), dried (Na₂SO₄) and concentrated under reduced pressure to yield the crude title compound as a white solid (20.3 g, 96%).

### tert-Butyl N-methyl-N-[(1r,4r)-4-(hydroxymethyl)cyclohexyl]carbamate

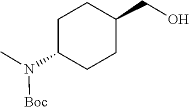 [SMILES: CN(C(=O)OC(C)(C)C)[C@H]1CC[C@H](CO)CC1]

To a stirred solution of (1r,4r)-4-[(tert-butoxycarbonyl)(methyl)amino]cyclohexane-1-carboxylic acid (20.3 g, 78.9 mmol, 1.0 eq.) in THE (500 mL) at 0° C. was added borane-THF complex (13.6 g, 158 mmol, 2.0 eq.). The resulting mixture was stirred overnight at room temperature then quenched with water/ice (400 mL). The resulting mixture was concentrated under reduced pressure then extracted with dichloromethane (3×300 mL). The combined organic layers were washed with brine (500 mL), dried (Na₂SO₄) and concentrated under reduced pressure to afford the crude title compound as a white solid (18.9 g, 98%).

### tert-Butyl N-methyl-N-[(1r,4r)-4-([(4-methylbenzenesulfonyl)oxy]methylcyclohexyl]carbamate

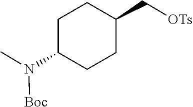 [SMILES: Cc1ccc(S(=O)(=O)OC[C@H]2CC[C@H](N(C)C(=O)OC(C)(C)C)CC2)cc1]

A solution of tert-butyl N-methyl-N-[(1r,4r)-4-(hydroxymethyl)cyclohexyl]carbamate (18.0 g, 74.0 mmol, 1.0 eq.), tosyl chloride (21.1 g, 111 mmol, 1.5 eq.) and triethylamine (30.8 mL, 222 mmol, 3.0 eq.) in dichloromethane (180 mL) was stirred for 4 hours at room temperature. The resulting mixture was filtered washing with dichloromethane (3×20 mL). The filtrate was concentrated under reduced pressure. The residue was purified by reverse phase flash column chromatography eluting with acetonitrile (50-68%) in water (0.1% formic acid), to afford the title compound as a white solid (23.0 g, 78%).

### tert-Butyl N-methyl-N-[(1r,4r)-4-[(methylsulfanyl)methyl]cyclohexyl]carbamate

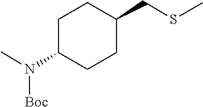

A solution of tert-butyl N-methyl-N-[(1r,4r)-4-([(4-methylbenzenesulfonyl)oxy]methylcyclohexyl]carbamate (12.0 g, 30.2 mmol, 1.0 eq.) and (methylsulfanyl)sodium (8.46 g, 121 mmol, 4.0 eq.) in methanol (120 mL) was stirred for 2 hours at room temperature. The resulting mixture was extracted with ethyl acetate (3×150 mL). The combined organic layers were washed with brine (150 mL), dried (Na₂SO₄) and concentrated under reduced pressure to yield the crude title compound as a white semi-solid (7.58 g, 92%).

### tert-Butyl N-methyl-N-[(1r,4r)-4-(methanesulfonylmethyl)cyclohexyl]carbamate

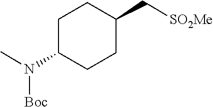 [SMILES: CN(C(=O)OC(C)(C)C)[C@H]1CC[C@H](CS(C)(=O)=O)CC1]

To a stirred solution of tert-butyl N-methyl-N-[(1r,4r)-4-[(methylsulfanyl)methyl]cyclohexyl]carbamate (7.00 g, 25.6 mmol, 1.0 eq.) in THF (70 mL) and water (70 mL) at 0° C. was added oxone (25.8 g, 154 mmol, 6.0 eq.). The resulting mixture was stirred for 2 hours at room temperature then extracted with ethyl acetate (3×100 mL). The combined organic layers were washed with brine (100 mL), dried (Na₂SO₄) and concentrated under reduced pressure to yield the crude title compound as a colourless oil (7.15 g, 91%).

### (1r,4r)-4-(Methanesulfonylmethyl)-N-methylcyclohexan-1-amine

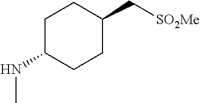

A solution of tert-butyl N-methyl-N-[(1r,4r)-4-(methanesulfonylmethyl)cyclohexyl]carbamate (2.00 g, 6.55 mmol, 1.0 eq.) and hydrogen chloride in methanol (20 mL) was stirred for 2 hours at room temperature then concentrated under reduced pressure, to afford the crude title compound as a white solid (1.59 g, 119%).

### N-Methyl-3-nitro-N-[1r,4r)-4-(methanesulfonylmethyl)cyclohexyl]aniline

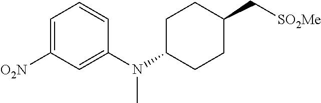 [SMILES: CN(c1cccc([N+](=O)[O-])c1)[C@H]1CC[C@H](CS(C)(=O)=O)CC1]

A solution of (1r,4r)-4-(methanesulfonylmethyl)-N-methylcyclohexan-1-amine (1.60 g, 7.79 mmol, 1.0 eq.), 3-bromo-1-nitrobenzene (1.73 g, 8.57 mmol, 1.1 eq.), cesium carbonate (5.08 g, 15.6 mmol, 2.0 eq.), Ruphos (364 mg, 0.779 mmol, 0.1 eq.) and Pd₂(dba)₃ (357 mg, 0.390 mmol, 0.05 eq.) in dioxane (16 mL) was stirred overnight at 80° C. The resulting mixture was concentrated under reduced pressure. The residue was purified by flash column chromatography eluting with ethyl acetate (40%) in 40-60 petroleum ether to afford the title compound as a reddish brown solid (1.74 g, 68%).

### N¹-Methyl-N¹-[(1r,4r)-4-(methanesulfonylmethyl)cyclohexyl]benzene-1,3-diamine

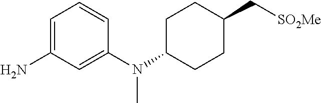 [SMILES: CN(c1cccc(N)c1)[C@H]1CC[C@H](CS(C)(=O)=O)CC1]

General Procedure 5 was applied N-methyl-3-nitro-N-[(1r,4r)-4-(methanesulfonylmethyl)cyclohexyl]aniline (300 mg, 0.919 mmol) and palladium on carbon (10%, 90.0 mg) in THE (30 mL) to yield the crude title compound as a brown semi-solid (300 mg, 110%).

### 3-Cyclopentyl-1-(2-[(3-(methyl[(1r,4r)-4-(methanesulfonylmethyl)cyclohexyl]aminophenyl)amino]-5-[2-(triisoprooylsilyl)ethynyl]pyrido[2,3-d]pyrimidin-7-ylurea

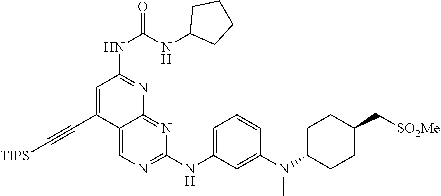

General Procedure 3 was applied to 3-cyclopentyl-1-(2-methanesulfinyl-5-[2-(triisopropylsilyl)ethynyl]pyrido[2,3-d]pyrimidin-7-ylurea (100 mg, 0.200 mmol), N¹-methyl-N¹-[(1r,4r)-4-(methanesulfonylmethyl)cyclohexyl]benzene-1,3-diamine (88.9 mg, 0.300 mmol) and trifluoroacetic acid (30.6 uL, 0.400 mmol) in 2-butanol (1.0 mL) stirred for 2 hours at 100° C. The residue was purified by reverse phase flash column chromatography eluting with acetonitrile (10-58%) in water (0.1% formic acid) to afford the title compound as an orange solid (76.9 mg, 52%).

### 3-Cyclopentyl-1-(5-ethynyl-2-[(3-(methyl[(1r,4r)-4-(methanesulfonylmethyl)cyclohexyl]aminophenyl)amino]pyrido[2,3-d]pyrimidin-7-ylurea

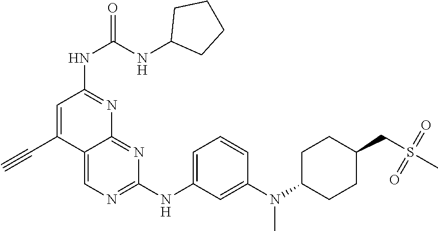 [SMILES: C#Cc1cc(NC(=O)NC2CCCC2)nc2nc(Nc3cccc(N(C)[C@H]4CC[C@H](CS(C)(=O)=O)CC4)c3)ncc12]

General Procedure 1 was applied to 3-cyclopentyl-1-(2-[(3-(methyl[(1r,4r)-4-(methanesulfonylmethyl)cyclohexyl]aminophenyl)amino]-5-[2-(triisopropylsilyl)ethynyl]pyrido[2,3-d]pyrimidin-7-ylurea (70.0 mg, 96.0 μmol) and potassium fluoride (55.6 mg, 0.960 mmol) in THE (1.0 mL), DMF (1.0 mL) and water (70 μL) stirred for 2 hours at 60° C. The residue was purified by reverse phase flash column chromatography eluting with acetonitrile (10-45%) in water (0.1% formic acid), to yield the title compound as a yellow solid (37.6 mg, 66%).

(ES, m/z) [M+H]⁺=576.20

¹H-NMR (400 MHz, DMSO-d₆) δ 9.99 (s, 1H), 9.87 (s, 1H), 9.44 (s, 1H), 9.08 (s, 1H), 7.70 (s, 1H), 7.18 (s, 1H), 7.12-7.00 (m, 2H), 6.54-6.42 (m, 1H), 5.05 (s, 1H), 4.13 (d, J=6.7 Hz, 1H), 3.55 (s, 1H), 3.08 (d, J=6.2 Hz, 2H), 2.99 (s, 3H), 2.73 (s, 3H), 2.02 (d, J=12.7 Hz, 2H), 1.91 (dd, J=12.5, 6.5 Hz, 3H), 1.79 (d, J=4.9 Hz, 2H), 1.68 (s, 4H), 1.62-1.47 (m, 4H), 1.34-1.20 (m, 2H).

### Example 122

### (1r,3r)-3-[(tert-butoxycarbonyl)(methyl)amino]cyclobutane-1-carboxylic acid

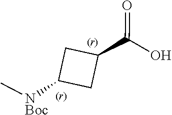 [SMILES: CN(C(=O)OC(C)(C)C)[C@H]1C[C@H](C(=O)O)C1]

To a solution of (1r,3r)-3-[(tert-butoxycarbonyl)amino]cyclobutane-1-carboxylic acid (10.0 g, 46.4 mmol, 1.0 eq.) in THE was added sodium hydride (60% in oil, 3.34 g) at 0° C. The mixture was stirred for 15 minutes then methyl iodide (19.8 g, 139 mmol, 3.0 eq.) was added and the mixture warmed to room temperature and stirred for 16 hours. The reaction was quenched with water/ice at 0° C. then concentrated under reduced pressure. The resulting residue was washed with MTBE (2×200 mL) then acidified to pH 4 with aqueous hydrochloric acid (2 M). The resulting mixture was extracted with dichloromethane (2×300 mL). The combined organic layers were washed with brine (2×100 mL), dried (Na₂SO₄) and concentrated under reduced pressure to yield the title compound as a white solid (8.00 g, 75%).

### tert-butyl N-methyl-N-[(1r,3r)-3-(hydroxymethyl)cyclobutyl]carbamate

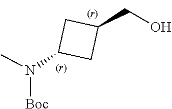

To a stirred solution of (1r,3r)-3-[(tert-butoxycarbonyl)(methyl)amino]cyclobutane-1-carboxylic acid (8.00 g, 34.9 mmol, 1.0 eq.) in THF (240 mL) was added borane-THF complex (6.68 mL, 69.8 mmol, 2.0 eq.) dropwise at 0° C. The resulting mixture was stirred overnight at room temperature under a hydrogen atmosphere. The reaction was quenched with water/ice then extracted with ethyl acetate (2×300 mL). The combined organic layers were washed with brine (2×200 mL), dried (Na₂SO₄) and concentrated under reduced pressure to yield the title compound as a colourless oil (8.00 g, crude).

### tert-Butyl N-methyl-N-[(1r,3r)-3-([(4-methylbenzenesulfonyl)oxy]methylcyclobutyl]carbamate

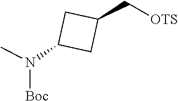

To a stirred mixture of tert-butyl N-methyl-N-[(1r,3r)-3-(hydroxymethyl)cyclobutyl]carbamate (8.00 g, 37.2 mmol, 1.0 eq.) and triethylamine (12.9 mL, 92.9 mmol, 2.5 eq.) in dichloromethane (80 mL) was added tosyl chloride (7.79 g, 40.9 mmol, 1.1 eq.) in portions at room temperature. The resulting mixture was stirred overnight at room temperature. The residue was purified by reverse phase flash column chromatography eluting acetonitrile (30-80%) in water (0.1% trifluoroacetic acid), to yield the title compound as a white solid (4.00 g, 29%).

### tert-Butyl N-methyl-N-[(1r,3r)-3-[(methylsulfanyl)methyl]cyclobutyl]carbamate

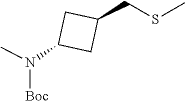

A mixture of tert-butyl N-methyl-N-[(1r,3r)-3-([(4-methylbenzenesulfonyl)oxy]methylcyclobutyl]carbamate (4.00 g, 10.8 mmol, 1.0 eq.) and sodium ethanethiolate (3.03 g, 43.3 mmol, 4.0 eq.) in methanol (40 mL) was stirred overnight at room temperature. The resulting mixture was extracted with ethyl acetate (2×200 mL). The combined organic layers were washed with brine (2×100 mL), dried (Na₂SO₄) and concentrated under reduced pressure to yield the title compound as a colourless solid (2.00 g, 75%).

### tert-Butyl N-methyl-N-[(1r,3r)-3-(methanesulfonylmethyl)cyclobutyl]carbamate

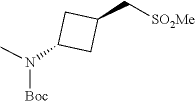 [SMILES: CN(C(=O)OC(C)(C)C)[C@H]1C[C@H](CS(C)(=O)=O)C1]

A mixture of tert-butyl N-methyl-N-[(1r,3r)-3-[(methylsulfanyl)methyl]cyclobutyl]carbamate (2.00 g, 8.15 mmol, 1.0 eq.) and oxone (5.48 g, 32.6 mmol, 4.0 eq.) in methanol (10 mL) and water (10 mL) were stirred for 3 hours at room temperature. The resulting mixture was extracted with ethyl acetate (2×100 mL). The combined organic layers were washed with brine (2×100 mL), dried (Na₂SO₄) and concentrated under reduced pressure to yield the title compound as a colourless solid (1.20 g, 53%).

### (1r,3r)-3-(Methanesulfonylmethyl)-N-methylcyclobutan-1-amine

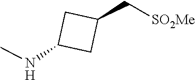

A solution of tert-butyl N-methyl-N-[(1r,3r)-3-(methanesulfonylmethyl)cyclobutyl]carbamate (1.20 g, 4.33 mmol, 1.0 eq.) in hydrogen chloride in 1,4-dioxane (12 mL) was stirred for 3 hours at room temperature then concentrated under reduced pressure to yield the title compound as a colourless solid (800 mg, crude).

### N-Methyl-3-nitro-N-[(1r,3r)-3-(methanesulfonylmethyl)cyclobutyl]aniline(

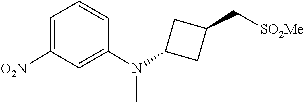 [SMILES: CN(c1cccc([N+](=O)[O-])c1)[C@H]1C[C@H](CS(C)(=O)=O)C1]

To a solution of (1r,3r)-3-(methanesulfonylmethyl)-N-methylcyclobutan-1-amine (800 mg, 4.51 mmol, 1.0 eq.) and 3-bromo-1-nitrobenzene (1.09 g, 5.42 mmol, 1.2 eq.) in dioxane (10 mL) was added cesium carbonate (2.94 g, 9.026 mmol, 2.0 eq.), RuPhos (210 mg, 0.451 mmol, 0.1 eq.) and Pd₂(dba)₃ (206 mg, 0.226 mmol, 0.05 eq.). After stirring overnight at 80° C., the reaction mixture was concentrated under reduced pressure. The residue was purified by flash column chromatography eluting with ethyl acetate (50%) in 40-60 petroleum ether to afford the title compound as a yellow solid (760 mg, 56%).

### N¹-Methyl-N¹-[(1r,3r)-3-(methanesulfonylmethyl)cyclobutyl]benzene-1,3-diamine

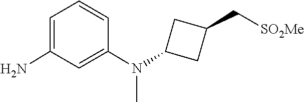 [SMILES: CN(c1cccc(N)c1)[C@H]1C[C@H](CS(C)(=O)=O)C1]

General Procedure 5 was applied to N-methyl-3-nitro-N-[(1r,3r)-3-(methanesulfonylmethyl)cyclobutyl]aniline (700 mg, 2.35 mmol) and palladium on carbon (140 mg) in methanol (7 mL) to yield the title compound as a yellow solid (500 mg, 79%).

### 3-Cyclopentyl-1-(2-[(3-(methyl[(1r,3r)-3-(methanesulfonylmethyl)cyclobutyl]aminophenyl)amino]-5-[2-(triisopropylsilyl)ethynyl]pyrido[2,3-d]pyrimidin-7-ylurea

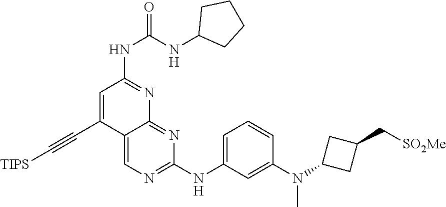

General Procedure 3 was applied to N¹-methyl-N¹-[(1r,3r)-3-(methanesulfonylmethyl)cyclobutyl]benzene-1,3-diamine (120 mg, 0.447 mmol), 3-cyclopentyl-1-(2-methanesulfinyl-5-[2-(triisopropylsilyl)ethynyl]pyrido[2,3-d]pyrimidin-7-ylurea (268 mg, 0.536 mmol) and trifluoroacetic acid (102 mg, 0.894 mmol) in butan-2-ol (2.0 mL), stirred for 1 hour at 100° C. The crude product was purified by Prep-HPLC eluting with acetonitrile (30-57%) in water (0.1% formic acid) to afford the title compound as a yellow solid (75.0 mg, 24%).

### 3-Cyclopentyl-1-(5-ethynyl-2-[(3-(methyl[(1r,3r)-3-(methanesulfonylmethyl)cyclobutyl]aminophenyl)amino]pyrido[2,3-d]pyrimidin-7-ylurea

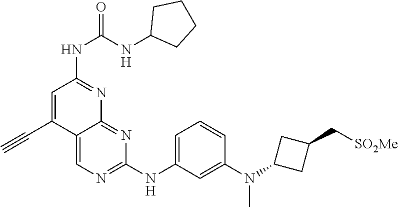 [SMILES: C#Cc1cc(NC(=O)NC2CCCC2)nc2nc(Nc3cccc(N(C)[C@H]4C[C@H](CS(C)(=O)=O)C4)c3)ncc12]

General Procedure 1 was applied to 3-cyclopentyl-1-(2-[(3-(methyl[(1r,3r)-3-(methanesulfonylmethyl)cyclobutyl]aminophenyl)amino]-5-[2-(triisopropylsilyl)ethynyl]pyrido[2,3-d]pyrimidin-7-ylurea (100 mg, 0.142 mmol) and potassium fluoride (82.5 mg, 1.42 mmol) in THF (0.5 mL), DMF (0.5 mL) and water (50 uL) stirred for 2 hours at room temperature. The crude product was purified by Prep-HPLC eluting with acetonitrile (30-57%) in water (0.1% formic acid) to afford the title compound as a yellow solid (51.0 mg, 62%).

(ES, m/z) [M+H]⁺=548.20

¹H-NMR (400 MHz, DMSO-d₆) δ 9.98 (d, J=22.4 Hz, 2H), 9.41 (s, 1H), 9.10 (s, 1H), 7.70 (s, 1H), 7.25-7.03 (m, 3H), 6.49 (dd, J=8.3, 2.4 Hz, 1H), 5.06 (s, 1H), 4.14 (dp, J=18.6, 6.8, 6.0 Hz, 2H), 3.44 (d, J=7.5 Hz, 2H), 2.95 (s, 3H), 2.79 (s, 3H), 2.70-2.58 (m, 1H), 2.40-2.21 (m, 4H), 1.90 (dt, J=12.8, 6.2 Hz, 2H), 1.77 (q, J=10.4, 7.6 Hz, 2H), 1.68 (d, J=6.7 Hz, 2H), 1.53 (q, J=6.9 Hz, 2H).

### Example 123

### N-Methyl-2-(methylsulfanyl)-5-[2-(triisopropylsilyl)ethynyl]pyrido[2,3-d]pyrimidin-7-amine

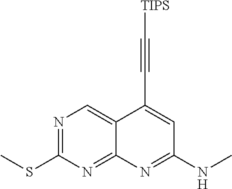

A solution of 7-chloro-2-(methylsulfanyl)-5-[2-(triisopropylsilyl)ethynyl]pyrido[2,3-d]pyrimidine (3.50 g, 8.93 mmol, 1.0 eq.) and 2M methylamine in THF (6.18 mL, 178 mmol, 20 eq.) in isopropanol (20 mL) was stirred for 3 hours at 80° C. The mixture was allowed to cool down to room temperature. The residue was purified by flash column chromatography, eluting with ethyl acetate (25%) in 40-60 petroleum ether to afford the title compound as a light yellow solid (2.56 g, 74%).

### 3-Cyclopentyl-1,3-dimethyl-1-[2-(methylsulfanyl)-5-[2-(triisopropylsilyl)ethynyl]pyrido[2,3-d]pyrimidin-7-yl]urea

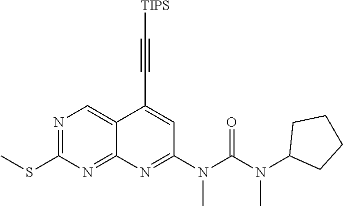

To a stirred solution of N-methyl-2-(methylsulfanyl)-5-[2-(triisopropylsilyl)ethynyl]pyrido[2,3-d]pyrimidin-7-amine (2.50 g, 6.47 mmol, 1.0 eq.) and triethylamine (5.39 mL, 38.8 mmol, 6.0 eq.) in dichloromethane (20 mL) was added triphosgene (3.84 g, 12.9 mmol, 2.0 eq.) portionwise at 0° C. The resulting mixture was stirred for 1 hour at 0° C. then N-methylcyclopentanamine (2.57 g, 25.8 mmol, 4.0 eq.) was added dropwise over 5 minutes at 0° C. The resulting mixture was stirred for an additional hour at room temperature then quenched with saturated aqueous sodium hydrogen carbonate (50 mL) at 0° C. The aqueous layer was extracted with ethyl acetate (100 mL). The organic layer was concentrated under reduced pressure. The residue was purified by flash column chromatography, eluting with ethyl acetate (12%) in 40-60 petroleum ether to afford the title compound as a light yellow solid (2.20 g, 66%).

### 3-Cyclopentyl-1-{2-methanesulfinyl-5-[2-(triisopropylsilyl)ethynyl]pyrido[2,3-d]pyrimidin-7-yl}-1,3-dimethyurea

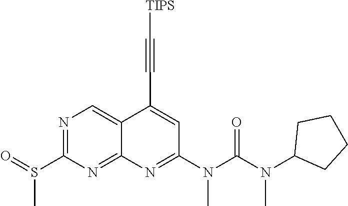

General Procedure 2 was applied to 3-cyclopentyl-1,3-dimethyl-1-[2-(methylsulfanyl)-5-[2-(triisopropylsilyl)ethynyl]pyrido[2,3-d]pyrimidin-7-yl]urea (2.00 g, 3.91 mmol) and m-CPBA (670 mg, 3.91 mmol) in dichloromethane (20 mL) to yield the title compound as a light yellow solid (2.00 g, 97%).

### 3-Cyclopentyl-1,3-dimethyl-1-(2-{[4-(4-methylpiperazin-1-yl)phenyl]amino}-5-[2-(triisopropylsilyl)ethynyl]pyrido[2,3-d]pyrimidin-7-yl)urea

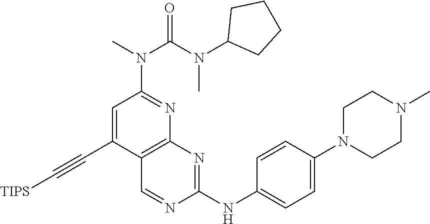

General Procedure 3 was applied to 3-cyclopentyl-1-{2-methanesulfinyl-5-[2-(triisopropylsilyl)ethynyl]pyrido[2,3-d]pyrimidin-7-yl}-1,3-dimethylurea (1.00 g, 1.89 mmol), 4-(4-methylpiperazin-1-yl)aniline (400 mg, 2.08 mmol) and trifluoroacetic acid (0.280 mL, 3.79 mmol) in 2-butanol (10 mL), stirring for 1 hour at 80° C. The residue was purified by reverse phase flash column chromatography eluting with acetonitrile (10-50%) in water (0.1% formic acid) to yield the title compound as a light yellow solid (320 mg, 26%).

### 3-Cyclopentyl-1-(5-ethynyl-2-{[4-(4-methylpiperazin-1-yl)phenyl]amino}pyrido[2,3-d]pyrimidin-7-yl)-1,3-dimethylurea

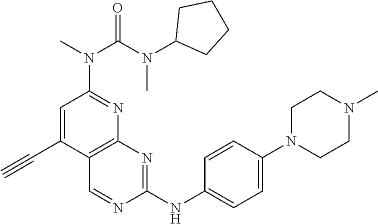 [SMILES: C#Cc1cc(N(C)C(=O)N(C)C2CCCC2)nc2nc(Nc3ccc(N4CCN(C)CC4)cc3)ncc12]

General Procedure 1 was applied to 3-cyclopentyl-1,3-dimethyl-1-(2-{[4-(4-methylpiperazin-1-yl)phenyl]amino}-5-[2-(triisopropylsilyl)ethynyl]pyrido[2,3-d]pyrimidin-7-yl)urea (200 mg, 0.305 mmol) and potassium fluoride (88.7 mg, 1.52 mmol) in DMF (2.0 mL), stirring for 1 hour at 60° C. The residue was purified by reverse phase flash column chromatography eluting with acetonitrile (10-50%) in water (0.1% formic acid), to yield the title compound as an orange solid (65.0 mg, 41%).

(ES, m/z) [M+H]⁺=499.40

¹H-NMR (400 MHz, DMSO-d₆) δ 9.81 (s, 1H), 9.04 (s, 1H), 7.74 (d, J=7.4 Hz, 2H), 6.92 (d, J=8.8 Hz, 2H), 6.83 (s, 1H), 4.98 (s, 1H), 4.42 (q, J=7.7 Hz, 1H), 3.25 (s, 3H), 3.08 (t, J=5.0 Hz, 4H), 2.80 (s, 3H), 2.46 (t, J=4.9 Hz, 4H), 2.22 (s, 3H), 1.86-1.75 (m, 2H), 1.66 (tt, J=10.3, 5.0 Hz, 4H), 1.52 (dd, J=10.8, 5.7 Hz, 2H).

### Example 124

### tert-Butyl N-[I(benzenesulfonyl)(cyclopentyl)methyl]carbamate

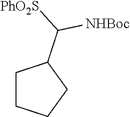

A solution of tert-butyl carbamate (47.7 g, 407 mmol, 1.0 eq.) in THF (150 mL) was treated with sodium benzenesulfinate icosahydrogen (67.6 g, 412 mmol, 1.01 eq.) followed by cyclopentanecarboxaldehyde (40.0 g, 407 mmol, 1.0 eq.) and then formic acid (100 mL). The resulting mixture was stirred overnight at room temperature. The residue was purified by flash column chromatography eluting with ethyl acetate (50%) in 40-60 petroleum ether to afford the title compound as a white solid (80.0 g, 58%).

### tert-Butyl N-(1-cyclopentyl-2-nitroethyl)carbamate

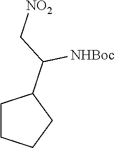 [SMILES: CC(C)(C)OC(=O)NC(C[N+](=O)[O-])C1CCCC1]

A solution of tert-butyl N-[(benzenesulfonyl)(cyclopentyl)methyl]carbamate (80.0 g, 235 mmol, 1.0 eq.) and potassium hydroxide (66.1 g, 1178 mmol, 5.0 eq.) in toluene (800 mL) was treated with nitromethane (71.9 g, 1178 mmol, 5.0 eq.) at 0° C. under N₂ atmosphere. The resulting mixture was stirred overnight at room temperature. The residue was purified by flash column chromatography eluting with ethyl acetate (50%) in 40-60 petroleum ether to afford the title compound as a white solid (20.0 g, 33%).

### tert-Butyl N-(2-amino-1-cyclopentylethyl)carbamate

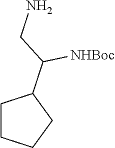 [SMILES: CC(C)(C)OC(=O)NC(CN)C1CCCC1]

General Procedure 5 was applied to tert-butyl N-(1-cyclopentyl-2-nitroethyl)carbamate (20.0 g, 77.4 mmol) in and palladium on carbon (4.00 g, 37.6 mmol) in ethanol (200 mL) to yield the title compound as a light yellow oil (15.0 g, 85%).

### Benzyl N-{2-[(tert-butoxycarbonyl)amino]-2-cyclopentylethyl}carbamate

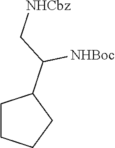 [SMILES: CC(C)(C)OC(=O)NC(CNC(=O)OCc1ccccc1)C1CCCC1]

A solution of tert-butyl N-(2-amino-1-cyclopentylethyl)carbamate (10.0 g, 43.8 mmol, 1.0 eq.) and sodium hydrogen carbonate (7.36 g, 87.6 mmol, 2.0 eq.) in dichloromethane/water (1/1, 100 mL) was treated with benzyl chloroformate (8.97 g, 52.5 mmol, 1.2 eq.). The resulting mixture was stirred for 2 hours at room temperature. The residue was purified by flash column chromatography eluting with ethyl acetate (50%) in 40-60 petroleum ether to afford the title compound as a white solid (4.50 g, 28%).

### Benzyl N-(2-amino-2-cyclopentylethyl)carbamate

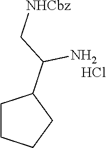 [SMILES: NC(CNC(=O)OCc1ccccc1)C1CCCC1 ; Cl]

A solution of benzyl N-{2-[(tert-butoxycarbonyl)amino]-2-cyclopentylethyl}carbamate (4.50 g, 12.4 mmol, 1.0 eq.) and 4M hydrogen chloride in 1,4-dioxane (15.5 mL, 62.1 mmol, 5.0 eq.) in dichloromethane (50 mL) was stirred overnight at room temperature. The resulting mixture was concentrated under reduced pressure to yield the title compound as a white solid (3.20 g, 98%).

### Benzyl N-(2-cyclopentyl-2-{[2-(methylsulfanyl)-5-[2-(triisopropylsilyl)ethynyl]pyrido[2,3-d]pyrimidin-7-yl]amino}ethyl)carbamate

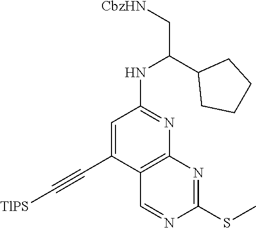

A solution of 7-chloro-2-(methylsulfanyl)-5-[2-(triisopropylsilyl)ethynyl]pyrido[2,3-d]pyrimidine (3.00 g, 7.65 mmol, 1.0 eq.), benzyl N-(2-amino-2-cyclopentylethyl)carbamate (3.01 g, 11.5 mmol, 1.5 eq.), tris(dibenzyldeneacetone)dipalladium(0) (350 mg, 0.383 mmol, 0.05 eq.), XantPhos (440 mg, 0.765 mmol, 0.1 eq.) and potassium carbonate (2.33 g, 16.8 mmol, 2.2 eq.) in dioxane (20 mL) was stirred for 2 hours at 100° C. under nitrogen atmosphere. The mixture was allowed to cool down to room temperature. The residue was purified by flash column chromatography eluting with ethyl acetate (25%) in 40-60 petroleum ether to afford the title compound as a light yellow oil (1.10 g, 23%).

### 1-Cyclopentyl-Nⁱ-[2-(methylsulfanyl)-5-[2-(triisopropylsilyl)ethynyl]pyrido[2,3-d]pyrimidin-7-yl]ethane-1.2-diamine

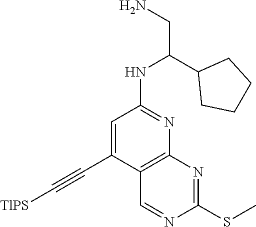

A solution of benzyl N-(2-cyclopentyl-2-{[2-(methylsulfanyl)-5-[2-(triisopropylsilyl)ethynyl]pyrido[2,3-d]pyrimidin-7-yl]amino}ethyl)carbamate (1.10 g, 1.78 mmol, 1.0 eq.) and hydrogen bromide in acetic acid (40%) (720 mg, 8.90 mmol, 5.0 eq.) in dichloromethane (20 mL) was stirred overnight at room temperature. The reaction mixture was neutralized to pH 9 with saturated aqueous sodium carbonate then extracted with dichloromethane (3×100 mL). The resulting mixture was concentrated under reduced pressure to yield the title compound as a yellow solid (800 mg, 93%).

### 5-Cyclopentyl-1-[2-(methylsulfanyl)-5-[2-(triisopropylsilyl)ethynyl]pyrido[2,3-d]pyrimidin-7-yl]imidazolidin-2-one

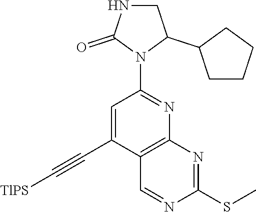

A solution of 1-cyclopentyl-N¹-[2-(methylsulfanyl)-5-[2-(triisopropylsilyl)ethynyl]pyrido[2,3-d]pyrimidin-7-yl]ethane-1,2-diamine (800 mg, 1.65 mmol, 1.0 eq.), diisopropylethylamine (427 mg, 3.31 mmol, 2.0 eq.) and CDI (348 mg, 2.15 mmol, 1.3 eq.) in DMF (10 mL) was stirred for 1 hour at 60° C. The resulting mixture was diluted with water (100 mL), extracted with ethyl acetate (2×100 mL) and concentrated under reduced pressure. The residue was purified by flash column chromatography eluting with ethyl acetate (25%) in 40-60 petroleum ether to afford the title compound as a yellow solid (380 mg, 45%).

### 5-Cyclopentyl-1-[2-methanesulfinyl-5-[2-(triisopropylsilyl)ethynyl]pyrido[2,3-d]pyrimidin-7-yl]imidazolidin-2-one

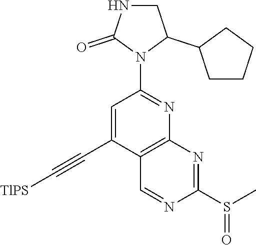

General Procedure 2 was applied to 5-cyclopentyl-1-[2-(methylsulfanyl)-5-[2-(triisopropylsilyl)ethynyl]pyrido[2,3-d]pyrimidin-7-yl]imidazolidin-2-one (350 mg, 0.687 mmol) and m-CPBA (178 mg, 1.03 mmol) in dichloromethane (5.0 mL) to yield the title compound as a yellow solid (345 mg, 95%).

### 5-Cyclopentyl-1-(2-{[4-(4-methylpiperazin-1-yl)phenyl]amino}-5-[2-(triisopropylsilyl)ethynyl]pyrido[2,3-d]pyrimidin-7-yl)imidazolidin-2-one

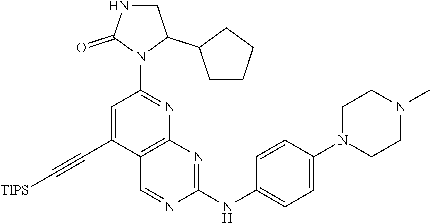

General Procedure 3 was applied to 5-cyclopentyl-1-{2-methanesulfinyl-5-[2-(triisopropylsilyl)ethynyl]pyrido[2,3-d]pyrimidin-7-yl}imidazolidin-2-one (345 mg, 0.656 mmol), 4-(4-methylpiperazin-1-yl)aniline (150 mg, 0.787 mmol) and trifluoroacetic acid (146 uL, 1.97 mmol) in 2-methyl-2-butanol (5.0 mL), stirred for 1 hour at 100° C. The residue was purified by reverse-phase flash column chromatography eluting with acetonitrile (10-35%) in water (0.1% formic acid), to afford the title compound as a yellow solid (200 mg, 46%). 5-Cyclopentyl-1-(5-ethynyl-2-{[4-(4-methylpiperazin-1-yl)phenyl]amino}pyrido[2,3-d]pyrimidin-7-yl)imidazolidin-2-one

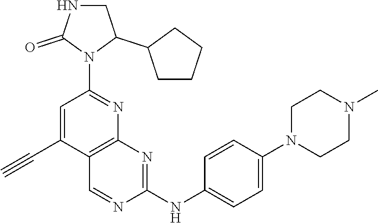 [SMILES: C#Cc1cc(N2C(=O)NCC2C2CCCC2)nc2nc(Nc3ccc(N4CCN(C)CC4)cc3)ncc12]

General Procedure 1 was applied to 5-cyclopentyl-1-(2-{[4-(4-methylpiperazin-1-yl)phenyl]amino}-5-[2-(triisopropylsilyl)ethynyl]pyrido[2,3-d]pyrimidin-7-yl)imidazolidin-2-one (110 mg, 0.168 mmol) and potassium fluoride (48.9 mg, 0.840 mmol) in DMF (2.0 mL), stirred for 30 minutes at 60° C. The crude product was purified by Prep-HPLC eluting with acetonitrile (18-40%) in water(0.1% formic acid) to afford the title compound as a red solid (57.0 mg, 66%).

(ES, m/z): [M+H]⁺=497.35

¹H-NMR (300 MHz, DMSO-d₆) δ 9.90 (s, 1H), 9.11 (s, 1H), 8.36 (s, 1H), 7.68 (s, 3H), 6.97-6.86 (m, 2H), 4.99 (s, 1H), 3.56 (s, 1H), 3.18 (dd, J=9.4, 2.8 Hz, 1H), 3.11 (t, J=5.1 Hz, 4H), 2.74 (s, 1H), 2.55 (t, J=4.9 Hz, 4H), 2.29 (s, 3H), 1.69-1.20 (m, 9H).

### TR-FRET Analysis

### Synthesis of Probe

### tert-butyl 4-(3-bromopropyl)piperazine-1-carboxylate

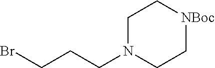 [SMILES: CC(C)(C)OC(=O)N1CCN(CCCBr)CC1]

To a stirred solution of 1-Boc-piperzine (1.00 g, 5.37 mmol) in dichloromethane (15 mL) at 0° C. was added triethylamine (596 mg, 5.90 mmol) and 1,3-dibromopropane (0.602 mL, 5.90 mmol). The reaction mixture was warmed to room temperature over 18 h. The reaction mixture was diluted with dichloromethane (30 mL) and washed with saturated aqueous sodium hydrogen carbonate solution (20 mL), water (10 mL) and brine (10 mL), dried (Na₂SO₄) and concentrated under reduced pressure. The crude material was purified by flash column chromatography eluting with ethyl acetate (30%) in 40-60 petroleum ether to yield the title compound as an oil (70%).

¹H NMR (500 MHz, Chloroform-d) δ 3.49 (t, J=6.6 Hz, 2H), 3.44 (t, J=5.2 Hz, 4H), 2.50 (t, J=6.9 Hz, 2H), 2.40 (t, J=5.1 Hz, 4H), 2.06-2.01 (m, 2H), 1.48 (s, 9H)

### tert-butyl 4-(3-((4-((3-chloro-4-fluorophenyl)amino)-7-methoxyquinazolin-6-yl)oxy)propyl)piperazine-1-carboxylate

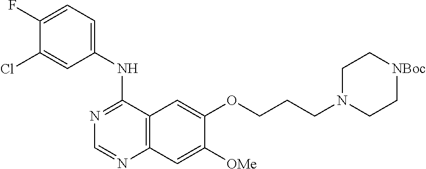 [SMILES: COc1cc2ncnc(Nc3ccc(F)c(Cl)c3)c2cc1OCCCN1CCN(C(=O)OC(C)(C)C)CC1]

To a stirred solution of 4-((3-chloro-4-fluorophenyl)amino)-7-methoxyquinazolin-6-ol (300 mg, 0.941 mmol) in DMF (3.0 mL) was added potassium carbonate (259 mg, 1.88 mmol) followed by tert-butyl 4-(3-bromopropyl)piperazine-1-carboxylate (346 mg, 1.12 mmol). The reaction mixture was heated at 80° C. for 5 h then concentrated Under reduced pressure. The residue was dissolved in dichloromethane (50 mL) and washed with brine (10 mL), dried (Na₂SO₄) and concentrated under reduced pressure. The crude material was purified by flash column chromatography to yield the title compound (49%).

(ES, m/z): [M+H]⁺=547

¹H NMR (500 MHz, Chloroform-d) δ 8.65 (s, 1H), 7.82 (dd, J=2.7, 6.5 Hz, 1H), 7.68 (s, 1H), 7.53 (ddd, J=2.7, 4.1, 8.9 Hz, 1H), 7.22 (s, 1H), 7.19 (s, 1H), 7.13 (t, J=8.7 Hz, 1H), 4.13 (t, J=6.6 Hz, 2H), 3.96 (s, 3H), 3.46-3.37 (m, 4H), 2.53 (t, J=7.0 Hz), 2.38 (t, J=5.1 Hz, 4H), 2.06 (dd, J=6.3, 13.1 Hz, 2H), 1.44 (s, 9H).

### N-(3-chloro-4-fluorophenyl)-7-methoxy-6-(3-(piperazin-1-yl)propoxy)quinazolin-4-amine

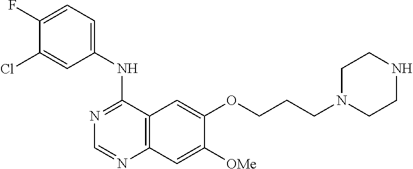 [SMILES: COc1cc2ncnc(Nc3ccc(F)c(Cl)c3)c2cc1OCCCN1CCNCC1]

Tert-butyl4-(3-((4-((3-chloro-4-fluorophenyl)amino)-7-methoxyquinazolin-6-yl)oxy)propyl)piperazine-1-carboxylate (234 mg, 0.422 mmol) in trifluoroacetic acid (1 mL) and dichloromethane (1 mL) was stirred at room temperature for 3 h. The reaction mixture was diluted with dichloromethane (20 mL), basified to pH 10, and extracted with dichloromethane (3×20 mL). The combined organic layers were dried (Na₂SO₄) and concentrated under reduced pressure to yield the title compound as yellow solid (180 mg, 91%).

¹H NMR (500 MHz, Chloroform-d) δ 8.66 (s, 1H), 7.91 (dd, J=6.5, 2.6 Hz, 1H), 7.55 (ddd, J=8.4, 3.9, 2.5 Hz, 1H), 7.45 (s, 1H), 7.25 (s, 1H), 7.19-7.13 (m, 2H), 4.19 (t, J=6.6 Hz, 2H), 4.00 (d, J=1.3 Hz, 3H), 2.93 (t, J=4.8 Hz, 4H), 2.59 (t, J=7.0 Hz, 2H), 2.55-2.44 (m, 4H), 2.12 (q, J=7.0 Hz, 2H).

### Methyl 4-azidobutanoate

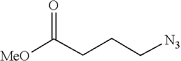

To a stirred solution of methyl 4-chlorobutyrate (500 mg, 3.66 mmol) in DMSO (3.0 mL) was added sodium azide (333 mg, 5.10 mmol). The reaction mixture was heated at 45° C. for 24 h then cooled down to room temperature and quenched with water (10 mL). The aqueous layer was extracted with diethyl ether (3×30 mL). The combined organic layers were dried (Na₂SO₄) and concentrated under reduced pressure to yield the title compound as a colourless oil (95%).

¹H NMR (500 MHz, Chloroform-d) b 3.70 (d, J=3.0 Hz, 3H), 3.37 (td, J=6.7, 2.7 Hz, 2H), 2.44 (td, J=7.3, 2.7 Hz, 2H), 1.93 (pd, J=7.1, 2.9 Hz, 2H).

### 4-azidobutanoic acid

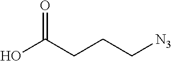

Methyl 4-azidobutanoate (500 mg, 3.49 mmol) was dissolved in 2M aqueous sodium hydroxide solution (6.99 mL), a few drops of methanol were added to the reaction and it was stirred at room temperature overnight. The reaction mixture was concentrated under reduced pressure to remove the methanol. The aqueous layer was washed with diethyl ether (3×20 mL), acidified with 6M HCl to pH 1 and then extracted with diethyl ether (3×35 mL). The combined organic layers were dried (MgSO₄) and concentrated under reduced pressure to yield the title compound as colourless oil (98%).

¹H NMR (500 MHz, Chloroform-d) b 3.40 (t, J=6.7 Hz, 2H), 2.49 (t, J=7.2 Hz, 2H), 1.93 (p, J=6.9 Hz, 2H). ¹³C NMR (126 MHz, Chloroform-d) b 178.81, 50.47, 30.91, 23.95

### 4-azido-1-(4-(3-((4-((3-chloro-4-fluorophenyl)amino)-7-methoxyquinazolin-6-yl)oxy)propyl)piperazin-1-yl)butan-1-one

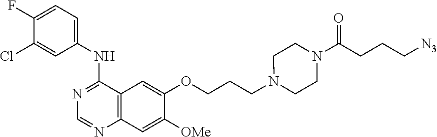 [SMILES: COc1cc2ncnc(Nc3ccc(F)c(Cl)c3)c2cc1OCCCN1CCN(C(=O)CCCN=[N+]=[N-])CC1]

To a stirred solution of 4-azidobutanoic acid (16.0 mg, 0.123 mmol) in dichloromethane (0.6 mL) was added N,N-diisoporpyl-N-ethylamine (43.4 mg, 0.336 mmol) followed by HATU (63.0 mg, 0.168 mmol). The reaction mixture stirred at rt for 10 minutes, N-(3-chloro-4-fluorophenyl)-7-methoxy-6-(3-(piperazin-1-yl)propoxy)quinazolin-4-amine (50.0 mg, 0.110 mmol) was added and the reaction mixture stirred at room temperature for 18 h. The reaction was diluted with dichloromethane (25 mL), washed with water (7 mL), dried (MgSO₄) and concentrated under reduced pressure. The crude material was purified by flash column chromatography using SiO₂ NH₂, eluting with ethyl acetate to yield the title compound as white solid (40.0 mg, 65%).

¹H NMR (500 MHz, Chloroform-d) δ 8.65 (s, 1H), 7.82 (dd, J=6.5, 2.7 Hz, 1H), 7.61 (s, 1H), 7.53 (ddd, J=8.9, 4.1, 2.7 Hz, 1H), 7.23 (s, 1H), 7.17 (s, 1H), 7.14 (t, J=8.8 Hz, 1H), 4.14 (t, J=6.5 Hz, 2H), 3.97 (s, 3H), 3.61 (t, J=5.0 Hz, 2H), 3.47 (dt, J=6.5, 3.9 Hz, 2H), 3.36 (t, J=6.4 Hz, 2H), 2.56 (t, J=7.0 Hz, 2H), 2.48-2.40 (m, 4H), 2.38 (d, J=7.2 Hz, 2H), 2.08 (q, J=6.8 Hz, 2H), 1.90 (p, J=6.8 Hz, 2H).

### 4-amino-1-(4-(3-((4-((3-chloro-4-fluorophenyl)amino)-7-methoxyquinazolin-6-yl)oxy)propyl)piperazin-1-yl)butan-1-one

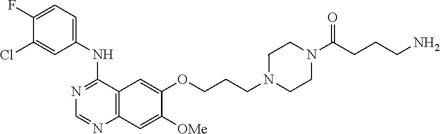 [SMILES: COc1cc2ncnc(Nc3ccc(F)c(Cl)c3)c2cc1OCCCN1CCN(C(=O)CCCN)CC1]

To a stirred solution of 4-azido-1-(4-(3-((4-((3-chloro-4-fluorophenyl)amino)-7-methoxyquinazolin-6-yl)oxy)propyl)piperazin-1-yl)butan-1-one (49.0 mg, 88.0 μmol) in methanol (2 mL) was added tin (II) chloride (25.0 mg, 0.132 mmol). The reaction was stirred at room temperature overnight, then concentrated under reduced pressure. The residue was suspended in ethyl acetate (15 mL) and washed with 5M aqueous potassium fluoride solution (10 mL). The aqueous layer was further extracted with ethyl acetate (10 mL). The combined organic layers were washed with brine, dried (Na₂SO₄) and concentrated under reduced pressure. The crude material was purified by flash column chromatography using amino silica eluting with methanol (0-20%) in dichloromethane to yield the titled compound as an oil (35 mg, 75%).

### Methyl (E)-3-(1H-pyrrol-2-yl)acrylate

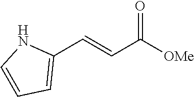 [SMILES: COC(=O)/C=C/c1ccc[nH]1]

To a stirred solution of pyrrole-2-carboxaldehyde (1.03 g, 10.4 mmol) in dichloromethane (50 mL) was added methyl (triphenylphosphoranyldene)acetate (7.07 g, 20.7 mmol) and the reaction stirred at room temperature for 24 h. The reaction mixture was concentrated under reduced pressure. The crude material was purified by flash column chromatography eluting with ethyl acetate (20-30%) in 40-60 petroleum ether to yield the title compound as an off white solid (60%).

¹H NMR (500 MHz, Chloroform-d) δ 8.96 (s, 1H), 7.51 (d, J=15.9 Hz, 1H), 6.86 (td, J=1.4, 2.8 Hz, 1H), 6.49 (ddd, J=1.4, 2.5, 3.8 Hz, 1H), 6.21 (dt, J=2.5, 3.7 Hz, 1H), 5.98 (d, J=15.9 Hz, 1H), 3.71 (s, 3H).

### Methyl 3-(1H-pyrrol-2-ylpropanoate

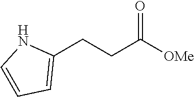 [SMILES: COC(=O)CCc1ccc[nH]1]

To a stirred solution of methyl (E)-3-(1H-pyrrol-2-yl)acrylate (800 mg, 5.29 mmol) in methanol (25 mL) was added 10% palldium on carbon (56.0 mg). The reaction was stirred under hydrogen for 3 h then filtered through Celite and concentrated under redcued pressure. The crude material was purified by flash column chromatography eluting with ethyl acetate (20%) in 40-60 petroleum ether to yield the title compound as pale yellow oil (650 mg, 81%).

(ES, m/z): [M+H]⁺=154

¹H NMR (500 MHz, Chloroform-d) b 8.46 (s, 1H), 6.60 (td, J=1.5, 2.7 Hz, 1H), 6.03 (q, J=2.8 Hz, 1H), 5.84 (ddd, J=1.6, 2.6, 3.3 Hz, 1H), 3.62 (s, 3H), 2.84 (t, J=6.8 Hz, 2H), 2.57 (t, J=6.8 Hz, 2H).

### 4,4-Difluoro-1,3-dimethyl-4-bora-3a,4a-diaza-s-indacene-2-propionic acid methyl ester

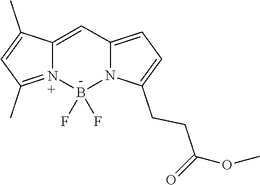

To a stirred solution of methyl 3-(1H-pyrrol-2-yl)propanoate (500 mg, 3.26 mmol), 3,5-dimethylpyrrole-2-carboxaldehyde (442 mg 3.59 mmol) in dichloromethane (25 mL) at 0° C. was added phosphorus (v) oxychloride (335 μL, 3.59 mmol) dropwise. After stirring for 30 minutes at 0° C. the reaction was stirred for 7 h at room temperature. The resultant dark solution was cooled down to 0° C. and BF₃—OEt₂ (1.59 mL, 13.0 mmol) and N,N-diisopropyl-N-ethylamine (2.15 mL, 13.0 mmol) were added and the mixture stirred overnight at room temperature. The reaction was quenched with water (50 mL) then filtered through Celite, washing with dichloromethane (50 mL). The filtrate was diluted with dichloromethane (50 mL) and separated. The aqueous layer was extracted with dichloromethane (3×30 mL). The combined organic layers were dried (Na₂SO₄) and concentrated under reduced pressure. The crude material was purified by flash column chromatography eluting with dichloromethane to yield the title compound as dark red solid (680 mg, 75%).

(ES, m/z): [M+H]⁺=307

¹H NMR (500 MHz, Chloroform-d): 5=7.09 (s, 1H), 6.89 (d, J=3.9 Hz, 1H), 6.27 (d, J=4.0 Hz, 1H), 6.12 (s, 1H), 3.70 (s, 3H), 3.31 (t, J=7.6 Hz, 2H), 2.78 (t, J=7.5 Hz, 2H), 2.58 (s, 3H), 2.26 ppm (s, 3H).

### 4,4-difluoro-1,3-dimethyl-4-bora-3a,4a-diaza-s-indacene-2-propionic acid

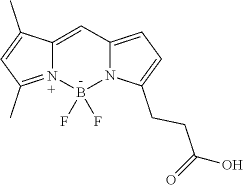

A solution of 4,4-Difluoro-1,3-dimethyl-4-bora-3a,4a-diaza-s-indacene-2-propionic acid methyl ester (650 mg) and concentrated hydrochloric acid in THF and water was stirred at room temperature for 24 h. Dichloromethane (150 mL) was added and the phases separated. The aqueous layer was extracted with dichloromethane (2×150 mL). The combined organic layers were washed with brine, dried (MgSO₄) and concentrated under reduced pressure. The crude material was purified by flash column chromatography eluting with dichloromethane (1% acetic acid) to yield the title compound as red solid (486 mg, 78%).

(ES, m/z): [M+H]⁺=293

¹H NMR (500 MHz, DMSO-d⁶): δ=12.30 (s, 1H), 7.70 (s, 1H), 7.09 (d, J=4.0 Hz, 1H), 6.38 (d, J=4.0 Hz, 1H), 6.31 (s, 1H), 3.10-3.05 (t, J=7.1 Hz, 2H), 2.64 (t, J=8.5 Hz, 2H), 2.47 (s, 3H), 2.26 ppm (s, 3H); ¹³C NMR (126 MHz, DMSO-d⁶): δ=173.4, 159.5, 156.9, 144.3, 134.5, 133.0, 128.8, 125.4, 120.4, 116.5, 32.3, 23.5, 14.5, 11.0

### N-(4-(4-(3-((4-((3-chloro-4-fluorophenyl)amino)-7-methoxyquinazolin-6-yl)oxy)propyl)piperazin-1-yl)-4-oxobutyl)-3-(5,5-difluoro-7,9-dimethyl-5H-514,614-dipyrrolo[1,2-c:2′,1′-f][1,3,2] diazaborinin-3-y)propanamide

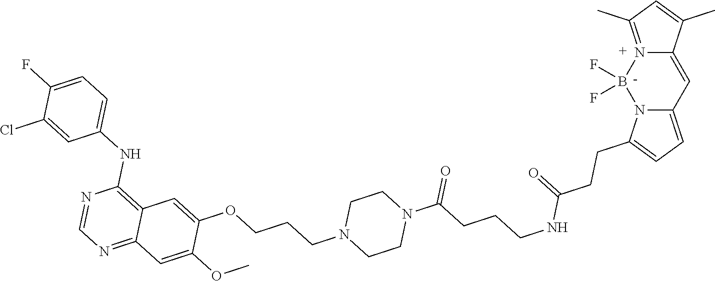

To a stirred solution of 4,4-difluoro-1,3-dimethyl-4-bora-3a,4a-diaza-s-indacene-2-propionic acid (38.0 mg, 0.136 mmol) in DMF (2 mL) was added N,N-diisopropyl-N-ethylamine (50.1 mg, 0.389 mmol) and HATU (74.0 mg, 0.194 mmol). The reaction was stirred at room temperature for 30 minutes then 4-amino-1-(4-(3-((4-((3-chloro-4-fluorophenyl)amino)-7-methoxyquinazolin-6-yl)oxy)propyl)piperazin-1-yl)butan-1-one (69.0 mg, 0.129 mmol) was added and the reaction mixture stirred overnight. The reaction mixture was concentrated under reduced pressure. The residue was dissolved in dichloromethane (30 mL), washed with saturated aqueous sodium hydrogen carbonate solution (2×10 mL). The aqueous layer was back extracted, the combined organic layers were dried (Na₂SO₄) and concentrated under reduced pressure. The crude material was purified by flash column chromatography using by amino silica, eluting with methanol in ethyl acetate to yield the title compound as red solid (62%).

¹H NMR (500 MHz, Chloroform-d) δ 8.63 (s, 1H), 8.00 (s, 1H), 7.89 (dd, J=6.6, 2.7 Hz, 1H), 7.57 (dt, J=9.1, 3.5 Hz, 1H), 7.37 (s, 1H), 7.22 (s, 1H), 7.10 (t, J=8.8 Hz, 1H), 7.03 (s, 1H), 6.82 (d, J=4.0 Hz, 1H), 6.22 (d, J=4.0 Hz, 1H), 6.14 (t, J=5.9 Hz, 1H), 6.03 (s, 1H), 4.19 (t, J=6.6 Hz, 2H), 3.98 (s, 3H), 3.70-3.65 (m, 2H), 3.48 (s, 2H), 3.25 (p, J=7.9, 7.1 Hz, 4H), 2.66 (t, J=6.8 Hz, 2H), 2.62-2.57 (m, 2H), 2.56-2.50 (m, 3H), 2.49 (s, 3H), 2.30 (t, J=6.8 Hz, 2H), 2.20 (s, 3H), 2.11 (t, J=6.7 Hz, 2H), 1.80 (p, J=6.7 Hz, 2H); ¹⁹F NMR (471 MHz, Chloroform-d) δ −121.39, −144.25 (dd, J=66.7, 33.0 Hz); ¹¹B NMR (160 MHz, Chloroform-d) δ 0.91 (t, J=34.4 Hz).

### TR-FRET Procedure

Compounds (dissolved to 100 mM in DMSO) were dispensed into black low volume 384 well assay plates (Corning) over a final concentration range of 100000, 30000, 10000, 3000, 1000, 300, 100, 30, 10, and 3 nM using an Echo 550 (Labcyte). Positive control compound and DMSO as a negative control were dispensed into the first and last well, respectively. Each well was backfilled with DMSO to a final volume of 200 nl, resulting in final assay DMSO concentrations of 1%. 19.8 μl of premixed solution containing final assay concentration of CtermHisTag-mEGFR (Wild type, Triple Mutant—C797S, L858R, T790M) (1.25 nM), Probe (N-(4-(4-(3-((4-((3-chloro-4-fluorophenyl)amino)-7-methoxyquinazolin-6-yl)oxy)propyl)piperazin-1-yl)-4-oxobutyl)-3-(5,5-difluoro-7,9-dimethyl-5H-514,614-dipyrrolo[1,2-c:2,1-f][1,3,2] diazaborinin-3-yl)propenamide) (100 nM), and Tb-anti-His Antibody 61HI2TLF (Cisbio Assay) (1:100 dilution) in a buffer containing 20 mM Tris pH 7.5, 100 mM NaCl, 100 μg/ml bovine serum albumin, was added to each well and incubated with shaking at room temperature for 30 mins. Plates were read using a PheraStar FS (BMG Labtech) at Ex337 nm Ex490/520 nm. The data were analysed using Graphpad Prism/Dotmatics Studies Software. Assays conducted in technical replicate and repeated as a biological duplicate.

TR-FRET Results

| Example Number | EGFR Triple Mutant (nM) | EGFR Wild-Type (uM) |
|---|---|---|
| 1 | 100-1,000 | >10 |
| 2 | 100-1,000 | >10 |
| 3 | 100-1,000 | >10 |
| 4 | 100-1,000 | >10 |
| 5 | 100-1,000 | >10 |
| 6 | 20-100 | 1-10 |
| 7 | 100-1,000 | >10 |
| 8 | 20-100 | 1-10 |
| 9 | 100-1,000 | >10 |
| 10 | 20-100 | 1-10 |
| 11 | 100-1,000 | >10 |
| 12 | 100-1,000 | >10 |
| 13 | 20-100 | >10 |
| 14 | 100-1,000 | >10 |
| 15 | 100-1,000 | >10 |
| 16 | 100-1,000 | >10 |
| 17 | 100-1,000 | >10 |
| 18 | 20-100 | >10 |
| 19 | 20-100 | 1-10 |
| 20 | 100-1,000 | >10 |
| 21 | 100-1,000 | >10 |
| 22 | 100-1,000 | >10 |
| 23 | 100-1,000 | >10 |
| 24 | 1,000-50,000 | >10 |
| 25 | 1,000-50,000 | >10 |
| 26 | 20-100 | 1-10 |
| 27 | 20-100 | <1 |
| 28 | <20 | >10 |
| 29 | 20-100 | 1-10 |
| 30 | <20 | 1-10 |
| 31 | <20 | 1-10 |
| 32 | <20 | 1-10 |
| 33 | 20-100 | >10 |
| 34 | <20 | 1-10 |
| 35 | <20 | <1 |
| 36 | <20 | 1-10 |
| 37 | 100-1,000 | >10 |
| 38 | <20 | <1 |
| 39 | <20 | <1 |
| 40 | <20 | <1 |
| 41 | <20 | <1 |
| 42 | <20 | <1 |
| 43 | <20 | <1 |
| 44 | <20 | <1 |
| 45 | 20-100 | <1 |
| 46 | <20 | <1 |
| 47 | 20-100 | <1 |
| 48 | <20 | <1 |
| 49 | 20-100 | <1 |
| 50 | 20-100 | <1 |
| 51 | <20 | <1 |
| 52 | 20-100 | 1-10 |
| 53 | <20 | <1 |
| 54 | <20 | 1-10 |
| 55 | <20 | <1 |
| 56 | 100-1,000 | >10 |
| 57 | <20 | <1 |
| 58 | 100-1,000 | >10 |
| 59 | <20 | <1 |
| 60 | <20 | <1 |
| 61 | <20 | <1 |
| 62 | <20 | <1 |
| 63 | 20-100 | <1 |
| 64 | <20 | 1-10 |
| 65 | 100-1,000 | >10 |
| 66 | 100-1,000 | >10 |
| 67 | 1,000-50,000 | >10 |
| 68 | <20 | 1-10 |
| 69 | <20 | >10 |
| 70 | <20 | <1 |
| 71 | <20 | <1 |
| 72 | <20 | <1 |
| 73 | <20 | <1 |
| 74 | 100-1,000 | 1-10 |
| 75 | <20 | <1 |
| 76 | <20 | <1 |
| 77 | <20 | <1 |
| 78 | 100-1,000 | 1-10 |
| 79 | <20 | <1 |
| 80 | <20 | <1 |
| 81 | <20 | <1 |
| 82 | <20 | >10 |
| 83 | <20 | <1 |
| 84 | <20 | <1 |
| 85 | 20-100 | <1 |
| 86 | 20-100 | <1 |
| 87 | 20-100 | <1 |
| 88 | 100-1,000 | >10 |
| 89 | 1,000-50,000 | >10 |
| 90 | 1,000-50,000 | >10 |
| 91 | <20 | <1 |
| 92 | <20 | <1 |
| 93 | 20-100 | >10 |
| 94 | 20-100 | <1 |
| 95 | 20-100 | <1 |
| 96 | <20 | <1 |
| 97 | 20-100 | >10 |
| 98 | 20-100 | <1 |
| 99 | <20 | <1 |
| 100 | 100-1,000 | >10 |
| 101 | 20-100 | 1-10 |
| 102 | <20 | 1-10 |
| 103 | 20-100 | <1 |
| 104 | 20-100 | <1 |
| 105 | 100-1,000 | >10 |
| 106 | 100-1,000 | 1-10 |
| 107 | 100-1,000 | 1-10 |
| 108 | 20-100 | >10 |
| 109 | 100-1,000 | >10 |
| 110 | 100-1,000 | >10 |
| 111 | <20 | 1-10 |
| 112 | <20 | <1 |
| 113 | 20-100 | >10 |
| 114 | <20 | <1 |
| 115 | <20 | 1-10 |
| 116 | <20 | 1-10 |
| 117 | 100-1,000 | 1-10 |
| 118 | 1,000-50,000 | >10 |
| 119 | 20-100 | <1 |
| 120 | 20-100 | <1 |
| 121 | 20-100 | <1 |
| 122 | <20 | <1 |
| 123 | 1,000-50,000 | >10 |
| 124 | 20-100 | <1 |

### HTRF Analysis

### Procedures

### HTRF Method (H1975):

H1975 cells (ATCC CRL-5908) were plated at 20,000 cells per well in 96 well plates and placed at 37° C. 5% CO₂. Once adhered (after 24h) cells were treated with compounds dissolved in DMSO at a final concentration of 0.1% DMSO in media. Compounds were diluted in media then added to cells for 3 hours in duplicate. Compound and media were removed from the cells, then 50ul of HTRF lysis buffer is added (lysis buffer was diluted from 4× stock to 1× in DI water, with 1% blocking agent also added). Cells were lysed on a plate shaker (2000 rpm) for 30 minutes at room temperature. pEGFR expression was monitored using the Cisbio Phospho-EGFR (Tyri068) cellular kit (64EG1 PEH). Total EGFR expression was monitored using the Cisbio Total EGFR cellular kit (64NG1 PEH) as per manufacturer's instructions. Fluorescence emission was read at two different wavelengths (665 nm and 620 nm) on a PheraStar. Results were calculated as ratio of pEGFR/total EGFR and then percentage of 0 uM control.

### HTRF Method (H1975 C797S):

H1975 C797S cells (ATCC CRL-5908) were genetically engineered by CRISPR editing of EGFR C797S in house and validated by NGS. Cells were plated at 20,000 cells per well in 96 well plates and placed at 37° C. 5% CO₂. Once adhered (after 24h) cells were treated with compounds dissolved in DMSO at a final concentration of 0.1% DMSO in media. Compounds were diluted in media then added to cells for 3 hours in duplicate. Compound and media were removed from the cells, then 50 ul of HTRF lysis buffer was added (lysis buffer was diluted from 4× stock to 1× in DI water, with 1% blocking agent also added). Cells were lysed on a plate shaker (2000 rpm) for 30 minutes at room temperature. pEGFR expression was monitored using the Cisbio Phospho-EGFR (Tyr1068) cellular kit (64EG1PEH). Total EGFR expression was monitored using the Cisbio Total EGFR cellular kit (64NG1PEH) as per manufacturer's instructions. Fluorescence emission was read at two different wavelengths (665 nm and 620 nm) on a PheraStar. Results were calculated as ratio of pEGFR/total EGFR and then percentage of 0 uM control.

### HTRF Method (A431):

Cells were plated at 20,000 cells per well in 96 well plates and placed at 37° C. 5% CO₂. Once adhered (after 24h) cells were treated with compounds dissolved in DMSO at a final concentration of 0.1% DMSO in media. Compounds were diluted in media then added to cells for 2.5 hours in duplicate, 100 ng/ml EGF (ThermoFisher, PHG0311 was is added to all compound treated cells as well as a control for 30 mins. Compound and media were removed from the cells, then 50ul of HTRF lysis bufferwas added (lysis bufferwas diluted from 4× stock to 1× in DI water, with 1% blocking agent also added). Cells were lysed on a plate shaker (2000 rpm) for 30 minutes at room temperature. pEGFR expression was monitored using the Cisbio Phospho-EGFR (Tyr1068) cellular kit (64EG1PEH). Total EGFR expression was monitored using the Cisbio Total EGFR cellular kit (64NG1PEH) as per manufacturer's instructions. Fluorescence emission was read at two different wavelengths (665 nm and 620 nm) on a PheraStar. Results were calculated as ratio of pEGFR/total EGFR and then percentage of OuM control.

### HTRF Data

HTRF Data

| Example Number | H1975 (nM) | H1975-C797S (nM) | A431 (nM) |
|---|---|---|---|
| 2 | 1,000-3,000 | >3,000 | >3,000 |
| 3 | 500-1,000 | 1,000-3,000 | >3,000 |
| 4 | >3,000 | >3,000 | >3,000 |
| 5 | >3,000 | >3,000 | >3,000 |
| 6 | 1,000-3,000 | >3,000 | >3,000 |
| 7 | >3,000 | >3,000 | >3,000 |
| 8 | 1,000-3,000 | >3,000 | >3,000 |
| 9 | >3,000 | >3,000 | >3,000 |
| 10 | >3,000 | >3,000 | >3,000 |
| 11 | >3,000 | >3,000 | >3,000 |
| 12 | >3,000 | >3,000 | >3,000 |
| 13 | 1,000-3,000 | >3,000 | >3,000 |
| 14 | >3,000 | >3,000 | >3,000 |
| 15 | 1,000-3,000 | >3,000 | >3,000 |
| 16 | 1,000-3,000 | >3,000 | >3,000 |
| 17 | 1,000-3,000 | 1,000-3,000 | >3,000 |
| 18 | >3,000 | >3,000 | >3,000 |
| 19 | 1,000-3,000 | 1,000-3,000 | >3,000 |
| 20 | >3,000 | >3,000 | >3,000 |
| 21 | >3,000 | >3,000 | >3,000 |
| 22 | >3,000 | >3,000 | >3,000 |
| 23 | >3,000 | >3,000 | >3,000 |
| 24 | >3,000 | >3,000 | >3,000 |
| 25 | >3,000 | >3,000 | >3,000 |
| 26 | 500-1,000 | 1,000-3,000 | >3,000 |
| 27 | 1,000-3,000 | >3,000 | >3,000 |
| 28 | >3,000 | >3,000 | >3,000 |
| 29 | >3,000 | >3,000 | >3,000 |
| 30 | 1,000-3,000 | 1,000-3,000 | >3,000 |
| 31 | 1,000-3,000 | 1,000-3,000 | >3,000 |
| 32 | 1,000-3,000 | 1,000-3,000 | >3,000 |
| 33 | >3,000 | >3,000 | >3,000 |
| 34 | >3,000 | 1,000-3,000 | >3,000 |
| 35 | 1,000-3,000 | >3,000 | >3,000 |
| 36 | 1,000-3,000 | >3,000 | >3,000 |
| 37 | >3,000 | >3,000 | >3,000 |
| 38 | <500 | <500 | <500 |
| 39 | <500 | <500 | >3,000 |
| 40 | <500 | <500 | >3,000 |
| 41 | <500 | <500 | 1,000-3,000 |
| 42 | <500 | <500 | >3,000 |
| 43 | <500 | <500 | >3,000 |
| 44 | <500 | <500 | 1,000-3,000 |
| 45 | 500-1,000 | 1,000-3,000 | >3,000 |
| 46 | >3,000 | >3,000 | >3,000 |
| 47 | 500-1,000 | 1,000-3,000 | >3,000 |
| 48 | <500 | <500 | >3,000 |
| 49 | 500-1,000 | 1,000-3,000 | >3,000 |
| 50 | <500 | 500-1,000 | >3,000 |
| 51 | <500 | <500 | >3,000 |
| 52 | >3,000 | >3,000 | >3,000 |
| 53 | 1,000-3,000 | >3,000 | >3,000 |
| 54 | >3,000 | >3,000 | >3,000 |
| 55 | <500 | <500 | >3,000 |
| 56 | 1,000-3,000 | >3,000 | >3,000 |
| 57 | >3,000 | >3,000 | >3,000 |
| 58 | >3,000 | >3,000 | >3,000 |
| 59 | 1,000-3,000 | 1,000-3,000 | >3,000 |
| 60 | <500 | <500 | <500 |
| 61 | <500 | <500 | >3,000 |
| 62 | <500 | <500 | 500-1,000 |
| 63 | <500 | 1,000-3,000 | <500 |
| 64 | <500 | 1,000-3,000 | <500 |
| 65 | 1,000-3,000 | >3,000 | >3,000 |
| 66 | >3,000 | >3,000 | >3,000 |
| 67 | >3,000 | >3,000 | >3,000 |
| 68 | <500 | 1,000-3,000 | >3,000 |
| 69 | 500-1,000 | 500-1,000 | >3,000 |
| 70 | 500-1,000 | >3,000 | 500-1,000 |
| 71 | 1,000-3,000 | >3,000 | 1,000-3,000 |
| 72 | 500-1,000 | 1,000-3,000 | >3,000 |
| 73 | <500 | <500 | >3,000 |
| 74 | >3,000 | >3,000 | >3,000 |
| 75 | <500 | <500 | <500 |
| 76 | <500 | <500 | <500 |
| 77 | 500-1,000 | >3,000 | >3,000 |
| 78 | >3,000 | >3,000 | >3,000 |
| 79 | <500 | <500 | >3,000 |
| 80 | >3,000 | <500 | 1,000-3,000 |
| 81 | 500-1,000 | 1,000-3,000 | 500-1,000 |
| 82 | >3,000 | >3,000 | >3,000 |
| 83 | <500 | <500 | >3,000 |
| 84 | <500 | <500 | >3,000 |
| 85 | >3,000 | <500 | 1,000-3,000 |
| 86 | >3,000 | 500-1,000 | 1,000-3,000 |
| 87 | >3,000 | <500 | 1,000-3,000 |
| 88 | >3,000 | >3,000 | >3,000 |
| 89 | >3,000 | >3,000 | >3,000 |
| 90 | >3,000 | >3,000 | >3,000 |
| 91 | 500-1,000 | 1,000-3,000 | 1,000-3,000 |
| 92 | 500-1,000 | 1,000-3,000 | 1,000-3,000 |
| 93 | >3,000 | 1,000-3,000 | 1,000-3,000 |
| 94 | 1,000-3,000 | 1,000-3,000 | 1,000-3,000 |
| 95 | 1,000-3,000 | 1,000-3,000 | 500-1,000 |
| 96 | 500-1,000 | 1,000-3,000 | 1,000-3,000 |
| 97 | >3,000 | >3,000 | >3,000 |
| 98 | >3,000 | >3,000 | 1,000-3,000 |
| 99 | <500 | <500 | >3,000 |
| 100 | 500-1,000 | 1,000-3,000 | >3,000 |
| 101 | 500-1,000 | >3,000 | >3,000 |
| 102 | >3,000 | >3,000 | >3,000 |
| 103 | <500 | <500 | 1,000-3,000 |
| 104 | 1,000-3,000 | 1,000-3,000 | >3,000 |
| 105 | >3,000 | >3,000 | >3,000 |
| 106 | >3,000 | 1,000-3,000 | 1,000-3,000 |
| 107 | 1,000-3,000 | 1,000-3,000 | >3,000 |
| 108 | >3,000 | >3,000 | 1,000-3,000 |
| 109 | >3,000 | >3,000 | 1,000-3,000 |
| 110 | >3,000 | >3,000 | >3,000 |
| 111 | 500-1,000 | 500-1,000 | >3,000 |
| 112 | 500-1,000 | 500-1,000 | >3,000 |
| 113 | 500-1,000 | 1,000-3,000 | >3,000 |
| 114 | <500 | 500-1,000 | 1,000-3,000 |
| 115 | 500-1,000 | >3,000 | >3,000 |
| 116 | <500 | 500-1,000 | >3,000 |
| 117 | >3,000 | >3,000 | >3,000 |
| 118 | >3,000 | >3,000 | >3,000 |
| 119 | <500 | <500 | |
| 120 | <500 | <500 | |
| 121 | 500-1,000 | <500 | |
| 122 | <500 | <500 | 1,000-3,000 |
| 123 | >3,000 | >3,000 | >3,000 |
| 124 | 500-1,000 | <500 | >3,000 |

1. A compound of formula I shown below, or a pharmaceutically acceptable salt thereof: 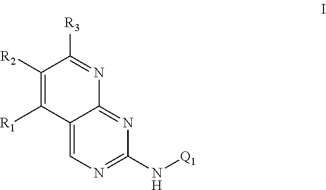
wherein:
R₁ is selected from (2C)alkynyl or (2C)alkenyl;
R₂ is selected from hydrogen, halo, cyano, (1-2C)alkyl or a group: 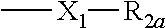
wherein:
X¹ is selected from the group consisting of —O—, —C(O)—, —C(O)—O—, —O—C(O)—, —S(O)₀₋₂—, —C(O)—N(R_{2b})—, —N(R_{2b})—C(O)—, —NR_{2b}—, —SO₂N(R_{2b})—, or —N(R_{2b})SO₂—, where R_{2b} is independently selected from the group consisting of hydrogen or (1-2C)alkyl; and
R₂ₐ is selected from hydrogen or (1-2C)alkyl, phenyl;
R₃ is hydrogen, halo, cyano or a group of the formula 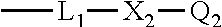
wherein:
L₁ is absent or (1-3C)alkylene;
X₂ is absent or is selected from the group consisting of —O—, —C(O)—, —NR₃ₐ—, —C(O)—O—, —O—C(O)—, —S(O)₀₋₂—, —C(O)—N(R₃ₐ)—, —N(R₃ₐ)—C(O)—, —O—C(O)—N(R₃ₐ)—, —N(R_{3b})—C(O)—O—, —N(R_{3b})—C(O)—NR₃ₐ—, —SO₂N(R₃ₐ)—, or —N(R₃ₐ)SO₂—, where R₃ₐ and R_{3b} are independently selected from the group consisting of hydrogen or (1-4C)alkyl;
Q₂ is selected from the group consisting of hydrogen, (1-6C)alkyl, (3-8C)cycloalkyl, (3-8C)cycloalkyl(1-4C)alkyl, aryl, aryl(1-4C)alkyl, heterocyclyl, heterocyclyl(1-4C)alkyl, heteroaryl and heteroaryl(1-4C)alkyl, wherein any alkyl, cycloalkyl, cycloalkyl-alkyl, aryl, arylalkyl, heterocyclyl, heterocyclyl-alkyl, heteroaryl or heteroaryl-alkyl in Q₂ is optionally substituted with one or more R_{3c} groups; and
each R₃ₑ group present is independently selected from the group consisting of hydroxy, cyano, oxo, halogen, (1-6C)alkyl, (1-6C)alkoxy, (3-6C)cycloalkyl, or (3-6C)cycloalkyl(1-2C)alkyl, —NR_{3d}R₃ₑ, —C(O)—R_{3d}, —C(O)—OR_{3d}, —O—C(O)—R_{3d}, —C(O)—NR_{3d}R₃ₑ, —N(R₃ₑ)C(O)—R_{3d}, —S(O)₀₋₂R_{3d}—, —S(O)₂NR_{3d}R₃ₑ, —N(R₃ₑ)—S(O)₂R_{3d}, phenyl, 5 or 6-membered heteroaryl, or 4 to 6-membered heterocyclyl,
wherein R_{3d} and R₃ₑ are each independently hydrogen, (1-6C)alkyl, (3-6C)cycloalkyl, or (3-6C)cycloalkyl(1-2C)alkyl;
and wherein any alkyl, alkoxy, cycloalkyl, cycloalkyl-alkyl, phenyl, heteroaryl or heterocyclyl group present in a R_{3c} group, or any alkyl cycloalkyl, or cycloalkyl-alkyl group present in a R_{3d} or R₃ₑ group, is optionally further substituted by one or more substituents independently selected from hydroxy, cyano, halogen, —OR_{3f}, —NR_{3f}R_{3g} and —C(O)—R_{3f}, wherein R_{3f} and R_{3g} are both independently selected from hydrogen and (1-2C)alkyl; and
Q₁ is selected from:
(i) a group of the formula: 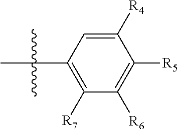
wherein:
R₄, R₅ and R₇ are each independently selected from hydrogen, halo, cyano, nitro, or a group of the formula: 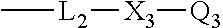
wherein:
L₂ is absent or (1-3C)alkylene;
X₃ is absent or is selected from the group consisting of —O—, —C(O)—, —NR₄ₐ—, —C(O)—O—, —O—C(O)—, —S(O)₀₋₂—, —C(O)—N(R₄ₐ)—, —N(R₄ₐ)—C(O)—, —O—C(O)—N(R₄ₐ)—, —N(R₄ₐ)—C(O)—O—, —N(R_{4b})—C(O)—NR₄ₐ—, —SO₂N(R₄ₐ)— or —N(R₄ₐ)SO₂—, where R₄ₐ and R_{4b} are independently selected from the group consisting of hydrogen or (1-4C)alkyl;
Q₃ is selected from the group consisting of hydrogen, (1-6C)alkyl, (3-8C)cycloalkyl, (3-8C)cycloalkyl(1-4C)alkyl, (2-6C)alkenyl, (2-6C)alkynyl, aryl, aryl(1-4C)alkyl, heterocyclyl, heterocyclyl(1-4C)alkyl, heteroaryl and heteroaryl(1-4C)alkyl, wherein any alkyl, cycloalkyl, cycloalkylalkyl, aryl, arylalkyl, heterocyclyl, heterocyclylalkyl, heteroaryl or heteroarylalkyl in Q₃ is optionally substituted with one or more R_{4c} groups; and
each R_{4c} group present is independently selected from the group consisting of hydroxy, cyano, oxo, halogen, (1-6C)alkyl, (1-6C)alkoxy, (3-6C)cycloalkyl, or (3-6C)cycloalkyl(1-2C)alkyl, —NR_{4d}R₄ₑ, —C(O)—R_{4d}, —C(O)—OR_{4d}, —O—C(O)—R_{4d}, —C(O)—NR_{4d}R₄ₑ, —N(R₄ₑ)C(O)—R_{4d}, —S(O)₀₋₂R_{4d}—, —S(O)₂NR_{4d}R₄ₑ, —N(R₄ₑ)—S(O)₂R_{4d}, phenyl, 5 or 6-membered heteroaryl, or 4 to 6-membered heterocyclyl,
wherein R_{4d} and R₄ₑ are each independently hydrogen, (1-6C)alkyl, (3-6C)cycloalkyl, or (3-6C)cycloalkyl(1-2C)alkyl;
and wherein any alkyl, alkoxy, cycloalkyl, cycloalkyl-alkyl, phenyl, heteroaryl or heterocyclyl group present in a R_{4c} group, or any alkyl, cycloalkyl or cycloalkyl-alkyl group present in a R_{4d} or R₄ₑ group, is optionally further substituted by one or more substituents independently selected from hydroxy, cyano, halogen, (1-2C)alkyl, —OR_{4f}, —NR_{4f}R_{4g}, —C(O)OR_{4f}, —OC(O)R_{4f}, —C(O)NR_{4f}R_{4g}, —NR_{4g}C(O)R_{4f}, —S(O)₀₋₂—R_{4f} and —C(O)—R_{4f}, wherein R_{4f} and R_{4g} are both independently selected from hydrogen and (1-2C)alkyl;
R₅ is:
a) hydrogen, halo, cyano;
b) a group of the formula: 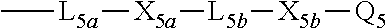
wherein:
L₅ₐ is absent or (1-3C)alkylene;
X₅ₐ is absent or is selected from the group consisting of —O—, —C(O)—, —NR₅ₐ₁—, —C(O)—O—, —O—C(O)—, —S(O)₀₋₂—, —C(O)—N(R₅ₐ₁)—, —N(R₅ₐ₁)—C(O)—, —O—C(O)—N(R₅ₐ₁), —N(R₅ₐ₁)—C(O)—O—, —N(R₅ₐ₂)—C(O)—NR₅ₐ₁—, —SO₂N(R₅ₐ₁)— or —N(R₅ₐ₁)SO₂—, where R₅ₐ₁ and R₅ₐ₂ are independently selected from the group consisting of hydrogen or (1-4C)alkyl;
L_{5b} is absent or (1-4C)alkylene;
X_{5b} is absent or is selected from the group consisting of —O—, —C(O)—, —NR_{5b1}—, —C(O)—O—, —O—C(O)—, —S(O)₀₋₂—, —C(O)—N(R_{5b1})—, —N(R_{5b1})—C(O)—, —C(O)—O—N(R_{5b})—, —N(R_{5b1})—O—C(O)—, —N(R_{5b2})—C(O)—NR_{5b1}—, —SO₂N(R_{5b1})— or —N(R_{5b1})SO₂—, where R_{5b1} and R_{5b2} are independently selected from the group consisting of hydrogen or (1-4C)alkyl;
Q₅ is selected from the group consisting of hydrogen, (1-6C)alkyl, (3-8C)cycloalkyl, (3-8C)cycloalkyl(1-4C)alkyl, phenyl, phenyl(1-2C)alkyl, 3 to 8-membered heterocyclyl, [3 to 8-membered heterocyclyl](1-2C)alkyl, 5 or 6-membered heteroaryl and 5 or 6-membered heteroaryl(1-2C)alkyl and wherein Q₅ is optionally substituted with one or more R_{5c} groups;
and each R_{5c} group present is independently selected from the group consisting of hydroxy, cyano, oxo, halogen, (1-6C)alkyl, (1-6C)alkoxy, (3-8C)cycloalkyl (including spiro-fused (3-8C)cycloalkyl), or (3-8C)cycloalkyl(1-2C)alkyl, —NR_{5d}R₅ₑ, —C(O)—R_{5d}, —C(O)—OR_{5d}, —O—C(O)—R_{5d}, —C(O)—NR_{5d}R₅ₑ, —N(R₅ₑ)C(O)—R_{5d}, —S(O)₀₋₂R_{5d}—, —S(O)₂NR_{5d}R₅ₑ, —N(R₅ₑ)—S(O)₂R_{5d}, phenyl, 5 or 6-membered heteroaryl, or 4 to 8-membered heterocyclyl,
wherein R_{5d} and R₅ₑ are each independently hydrogen, (1-6C)alkyl, (3-6C)cycloalkyl, or (3-6C)cycloalkyl(1-2C)alkyl;
and wherein any alkyl, alkoxy, cycloalkyl, cycloalkyl-alkyl, phenyl, heteroaryl or heterocyclyl group present in a R_{5c} group, or any alkyl, cycloalkyl or cycloalkyl-alkyl group present in a R_{5d} or R₅ₑ group, is optionally further substituted by one or more substituents independently selected from hydroxy, cyano, halo, R_{5f}, —OR_{5f}, —NR_{5f}R_{5g} and —C(O)—R_{5f}, wherein R_{5f} and R_{5g} are both independently selected from hydrogen, (1-4C)alkyl, hydroxy(1-4C)alkyl, halo(1-4C)alkyl, or (1-2C)alkoxy(1-4C)alkyl;
or R₄ and R₅, or R₅ and Re, or R₆ and R₇ are linked to form a fused phenyl, 5- or 6-membered heteroaryl or 5 to 8-membered heterocyclic ring, which is optionally substituted by one or more substituent groups selected from hydroxy, cyano, halo, (1-2C)alkyl, (1-2C)haloalkyl, (1-2C)alkoxy or (1-2C)haloalkoxy;
(ii) a group of the formula: 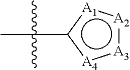
wherein:
A₁ is selected from N, NR_{1N}, O, S or CR₇;
A₂ is selected from N, NR_{1N}, O, S or CR₄;
A₃ is selected from N, NR_{1N}, O, S or CR₅;
A₄ is selected from N, NR_{1N}, O, S or CR₇;
R_{1N} is selected from hydrogen, (1-4C)alkyl, (2-4C)alkanoyl, phenyl, benzyl, (3-8C)cycloalkyl, (3-8C)cycloalkyl-(1-2C)alkyl, a carbon-linked 4 to 7 membered heterocyclyl, or 4 to 7 membered heterocyclyl(1-2C)alkyl, and wherein any alkyl, alkanoyl, cycloalkyl, cycloalkylalkyl, phenyl, benzyl, heterocyclyl, heterocyclylalkyl group is optionally substituted with one or more R_{5c} groups defined above;
R₄, R₅ and R₇ are as defined above;
and with the proviso that:
(i) only one of A₁, A₂, A₃ and A₄ can be O or S;
(ii) only one of A₁, A₂, A₃ and A₄ can be NR_{1N};
(iii) 1 to 4 of A₁, A₂, A₃ and A₄ can be N;
(iii) a group of the formula: 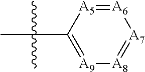
wherein:
A₅ is selected from N or CH;
As is selected from N or CR₄;
A₇ is selected from N or CR₅;
A₉ is selected from N or CR₅;
A₉ is selected from N or CR₇;
R₄, R₅, R₆ and R₇ are as defined above;
and with the proviso that one or two of As, Ae, A₇, A₈ and A₉ can be N;
(iv) a group of the formula: 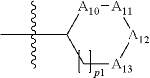
wherein:
p1 is 0 or 1;
A₁₀ is selected from NH, CH₂ or CHR₇;
A₁₁ is selected from NR_{4N}, CH₂ or CHR₄;
A₁₂ is selected from NR_{5N}, CH₂ or CHR₅;
A₁₃ is selected from NR_{6N}, CH₂ or CHR₆;
R₄, R₅, R₆ and R₁ are as defined above;
R_{4N} and R_{6N} are each independently selected from hydrogen or a group of the formula: 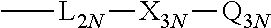
wherein:
L_{2N} is absent or (1-3C)alkylene;
X_{3N} is absent or is selected from the group consisting of:
(i) —C(O)—, —S(O)₀₋₂— or —C(O)—N(R₄ₐ)— when L₂ is absent; or
(ii) —O—, —C(O)—, —NR₄ₐ—, —C(O)—O—, —O—C(O)—, —S(O)₀₋₂—, —C(O)—N(R₄ₐ)—, —N(R₄ₐ)—C(O)—, —O—C(O)—N(R₄ₐ)—, —N(R₄ₐ)—C(O)—O—, —N(R_{4b})—C(O)—NR₄ₐ—, —SO₂N(R₄ₐ)— or —N(R₄ₐ)SO₂— when L₂ is (1-3C)alkylene;
wherein R₄ₐ and R_{4b} are as defined above;
Q_{3N} is selected from the group consisting of hydrogen, (1-6C)alkyl, (3-8C)cycloalkyl, (3-8C)cycloalkyl(1-4C)alkyl, (2-6C)alkenyl, (2-6C)alkynyl, aryl, aryl(1-4C)alkyl, heterocyclyl, heterocyclyl(1-4C)alkyl, heteroaryl and heteroaryl(1-4C)alkyl, wherein any alkyl, cycloalkyl, cycloalkylalkyl, aryl, arylalkyl, heterocyclyl, heterocyclylalkyl, heteroaryl or heteroarylalkyl in Q₃ is optionally substituted with one or more R_{4c} groups; and
each R₄ₑ group present is as defined above;
R_{5N} is:
a) hydrogen;
b) a group of the formula: 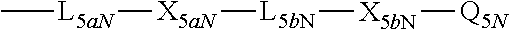
wherein:
L_{5aN} is absent or (1-3C)alkylene;
X_{5aN} is absent or is selected from the group consisting of:
(i) —C(O)—, —S(O)₀₋₂—, —C(O)—N(R₅ₐ₁)— when L_{5aN} is absent; or
(ii) —O—, —C(O)—, —NR₅ₐ₁—, —C(O)—O—, —O—C(O)—, —S(O)₀₋₂—, —C(O)—N(R₅ₐ₁), —N(R₅ₐ₁)C(O)—, —O—C(O)—N(R₅ₐ₁), —N(R₅ₐ)C(O)—O—, —N(R₅ₐ₂)—C(O)—NR₅ₐ₁—, —SO₂N(R₅ₐ)— or —N(R₅ₐ₁)SO₂— when L_{5aN} is present;
where R₅ₐ₁ and R₅ₐ₂ are as defined above;
L_{5bN} is absent or (1-40)alkylene;
X_{5bN} is absent or is selected from the group consisting of —O—, —C(O)—, —NR_{5b1}—, —C(O)—O—, —O—C(O)—, —S(O)₀₋₂—, —C(O)—N(R_{5b})—, —N(R_{5b})—C(O)—, —O—C(O)—N(R_{5b})—, —N(R_{5b1})—C(O)—O—, —N(R_{5b2})—C(O)—NR_{5b1}—, —SO₂N(R_{5b1})— or —N(R_{5b1})SO₂—, where R_{5b1} and R_{5b2} are independently selected from the group consisting of hydrogen or (1-4C)alkyl;
Q_{5N} is selected from the group consisting of:
(i) hydrogen;
(ii) (1-6C)alkyl, (3-8C)cycloalkyl, (3-8C)cycloalkyl(1-4C)alkyl, phenyl, phenyl(1-4C)alkyl, 5- or 6-membered heteroaryl, 5- or 6-membered heteroaryl(1-2C)alkyl;
(iii) a carbon-linked 4 to 8 membered heterocyclyl (when -L_{5aN}, X_{5aN}, L_{5bN}, and X_{5bN} are absent) or a 4 to 8 membered heterocyclyl (when one or more of -L_{5aN}, X_{5aN}, L_{5bN}, and X_{5bN} are present), or [4 to 8 membered heterocyclyl](1-2C)alkyl;
and wherein Q_{5N} is optionally substituted with one or more R_{5c} groups as defined above;
or R_{4N} and R_{5N}, or R_{5N} and R_{6N} are linked to form a fused phenyl, 5- or 6-membered heteroaryl or 5 to 8-membered heterocyclic ring, which is optionally substituted by one or more substituent groups selected from hydroxy, cyano, halo, (1-2C)alkyl, (1-2C)haloalkyl, (1-2C)alkoxy or (1-2C)haloalkoxy;
and with the proviso that one or two of A₁₀, A₁₁, A₁₂ and A₁₃ can be N;
(v) a group of the formula: 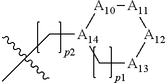
wherein:
p1, A₁₀, A₁₁, A₁₂, and A₁₃ are as defined above;
p2 is 1 to 6;
A₁₄ is C or N;
and with the proviso that one of A₁₀, A₁₁, A₁₂ and A₁₃ can be N.

2. A compound according to claim 1, or a pharmaceutically acceptable salt thereof, wherein R₁ is ethynyl.

3. A compound according to claim 1 or claim 2, or a pharmaceutically acceptable salt thereof, wherein R₂ is selected from hydrogen, halo, cyano, (1-2C)alkyl or a group: 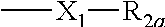
wherein:
X¹ is selected from the group consisting of —O—, —C(O)—, —C(O)—O—, —O—C(O)—, —S(O)₀₋₂—, —C(O)—N(R_{2b})—, —N(R_{2b})—C(O)— or —NR_{2b}—;
where R_{2b} is independently selected from the group consisting of hydrogen or (1-2C)alkyl; and
R₂ₐ is selected from hydrogen, (1-2C)alkyl or phenyl.

4. A compound according to any one of the preceding claims, or a pharmaceutically acceptable salt thereof, wherein R₂ is selected from hydrogen, halo, cyano or (1-2C)alkyl; optionally R₂ selected from hydrogen or methyl.

5. A compound according to any one of the preceding claims, or a pharmaceutically acceptable salt thereof, wherein R₃ is halo, cyano, or a group of the formula: 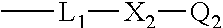
wherein:
L₁ is absent or (1-2C)alkylene;
X₂ is absent or is selected from the group consisting of —O—, —C(O)—, —NR₃ₐ—, —C(O)—O—, —O—C(O)—, —S(O)₀₋₂—, —C(O)—N(R₃ₐ)—, —N(R₃ₐ)—C(O)—, —O—C(O)—N(R₃ₐ)—, —N(R₃ₐ)—C(O)—O—, —N(R_{3b})—C(O)—NR₃ₐ—, —SO₂N(R₃ₐ)—, or —N(R₃ₐ)SO₂—, where R₃ₐ and R_{3b} are independently selected from the group consisting of hydrogen or (1-2C)alkyl;
Q₂ is selected from the group consisting of hydrogen, (1-4C)alkyl, (3-8C)cycloalkyl, (3-8C)cycloalkyl(1-2C)alkyl, phenyl, phenyl(1-4C)alkyl, 3- to 10-membered heterocyclyl, [3- to 10-membered heterocyclyl](1-2C)alkyl, 5- to 10-membered heteroaryl and [5- to 10-membered heteroaryl](1-2C)alkyl, wherein any alkyl, cycloalkyl, cycloalkyl-alkyl, phenyl, phenylalkyl, heterocyclyl, heterocyclyl-alkyl, heteroaryl or heteroaryl-alkyl in Q₂ is optionally substituted with one or more R₃, groups; and
each R_{3c} group present is independently selected from the group consisting of hydroxy, cyano, oxo, halogen, (1-4C)alkyl, (1-4C)alkoxy, (3-6C)cycloalkyl, or (3-6C)cycloalkyl(1-2C)alkyl, —NR_{3d}R₃ₑ, —C(O)—R_{3d}, —C(O)—OR_{3d}, —O—C(O)—R_{3d}, —C(O)—NR_{3d}R₃ₑ, —N(R₃ₑ)C(O)—R_{3d}, —S(O)₀₋₂R_{3d}—, —S(O)₂NR_{3d}R₃ₑ, —N(R₃ₑ)—S(O)₂R_{3d}, phenyl, 5 or 6-membered heteroaryl, or 4 to 6-membered heterocyclyl,
wherein R_{3d} and R₃ₑ are each independently hydrogen, (1-4C)alkyl, (3-6C)cycloalkyl, or (3-6C)cycloalkyl(1-2C)alkyl;
and wherein any alkyl, alkoxy, cycloalkyl, cycloalkyl-alkyl, phenyl, heteroaryl or heterocyclyl group present in a R₃, group, or any alkyl, cycloalkyl, or cycloalkyl-alkyl group present in a R_{3d} or R₃ₑ group, is optionally further substituted by one or more substituents independently selected from hydroxy, cyano, halogen, (1-2C)alkyl, —OR_{3f}, —NR_{3f}R_{3g} and —C(O)—R_{3f}, wherein R_{3f} and R_{3g} are both independently selected from hydrogen and (1-2C)alkyl.

6. A compound according to any one of the preceding claims, or a pharmaceutically acceptable salt thereof, wherein R₃ is halo, cyano, or a group of the formula 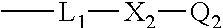
wherein:
L₁ is absent or (1-2C)alkylene;
X₂ is absent or is selected from the group consisting of —O—, —C(O)—, —NR₃ₐ—, —C(O)—O—, —O—C(O)—, —S(O)₀₋₂—, —C(O)—N(R₃ₐ)—, —N(R₃ₐ)—C(O)—, —N(R_{3b})—C(O)—O—, —N(R_{3b})—C(O)—NR₃ₐ—, —SO₂N(R₃ₐ)— or —N(R₃ₐ)SO₂—, where R₃ₐ is selected from the group consisting of hydrogen or (1-2C)alkyl;
Q₂ is selected from the group consisting of hydrogen, (1-4C)alkyl, (3-6C)cycloalkyl, (3-6C)cycloalkyl(1-2C)alkyl, phenyl, phenyl(1-4C)alkyl, 3- to 10-membered heterocyclyl, [3- to 10-membered heterocyclyl](1-2C)alkyl, 5- to 10-membered heteroaryl and [5- to 10-membered heteroaryl](1-2C)alkyl, wherein any alkyl, cycloalkyl, cycloalkyl-alkyl, phenyl, phenylalkyl, heterocyclyl, heterocyclyl-alkyl, heteroaryl or heteroaryl-alkyl in Q₂ is optionally substituted with one or more R₃ₑ groups; and
each R₃ₑ group present is independently selected from the group consisting of hydroxy, cyano, oxo, halogen, (1-4C)alkyl, (1-4C)alkoxy, (3-6C)cycloalkyl, or (3-6C)cycloalkyl(1-2C)alkyl, —NR_{3d}R₃ₑ, —C(O)—R_{3d}, —C(O)—OR_{3d}, —O—C(O)—R_{3d}, —C(O)—NR_{3d}R₃ₑ, —N(R₃ₑ)C(O)—R_{3d}, —S(O)₀₋₂R_{3d}—, —S(O)₂NR_{3d}R₃ₑ, —N(R₃ₑ)—S(O)₂R_{3d}, phenyl, 5 or 6-membered heteroaryl, or 4 to 6-membered heterocyclyl,
wherein R_{3d} and R₃ₑ are each independently hydrogen or (1-2C)alkyl;
and wherein any alkyl, alkoxy, cycloalkyl, cycloalkyl-alkyl, phenyl, heteroaryl or heterocyclyl group present in a R_{3c} group, or any alkyl group present in a R_{3d} or R₃ₑ group, is optionally further substituted by one or more substituents independently selected from hydroxy, cyano, halogen, (1-2C)alkyl, or —OR_{3f}, wherein R_{3f} is selected from hydrogen and (1-2C)alkyl.

7. A compound according to any one of the preceding claims, or a pharmaceutically acceptable salt thereof, wherein R₃ is cyano, or a group of the formula 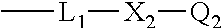
wherein:
L₁ is absent or (1-2C)alkylene;
X₂ is absent or is selected from the group consisting of —O—, —C(O)—, —NR₃ₐ—, —C(O)—O—, —O—C(O)—, —S(O)₀₋₂—, —C(O)—N(R₃ₐ)—, —N(R₃ₐ)—C(O)—, —N(R_{3b})—C(O)—O—, —N(R_{3b})—C(O)—NR₃ₐ—, —SO₂N(R₃ₐ)— or —N(R₃ₐ)SO₂—, where R₃ₐ is selected from the group consisting of hydrogen or (1-2C)alkyl;
Q₂ is selected from the group consisting of hydrogen, (1-4C)alkyl, (3-6C)cycloalkyl, (3-6C)cycloalkyl(1-2C)alkyl, phenyl, phenyl(1-4C)alkyl, 4- to 6-membered heterocyclyl, [4- to 6-membered heterocyclyl](1-2C)alkyl, 5- to 10-membered heteroaryl and [5- to 10-membered heteroaryl](1-2C)alkyl, wherein any alkyl, cycloalkyl, cycloalkyl-alkyl, phenyl, phenylalkyl, heterocyclyl, heterocyclyl-alkyl, heteroaryl or heteroaryl-alkyl in Q₂ is optionally substituted with one or more R_{3c} groups; and
each R_{3c} group present is independently selected from the group consisting of hydroxy, cyano, oxo, halogen, (1-4C)alkyl, (1-4C)alkoxy, —NR_{3d}R₃ₑ, —C(O)—R_{3d}, —C(O)—OR_{3d}, —O—C(O)—R_{3d}, —C(O)—NR_{3d}R₃ₑ, —N(R₃ₑ)C(O)—R_{3d}, —S(O)₀₋₂R_{3d}—, —S(O)₂NR_{3d}R₃ₑ, —N(R₃ₑ)—S(O)₂R_{3d}, phenyl, 5 or 6-membered heteroaryl, or 4 to 6-membered heterocyclyl,
wherein R_{3d} and R₃ₑ are each independently hydrogen or (1-2C)alkyl;
and wherein any alkyl, alkoxy, cycloalkyl, cycloalkyl-alkyl, phenyl, heteroaryl or heterocyclyl group present in a R₃ₑ group is optionally further substituted by one or more substituents independently selected from hydroxy, cyano, halogen, (1-2C)alkyl, or —OR_{3Y}, wherein R_{3f} is selected from hydrogen and (1-2C)alkyl.

8. A compound according to any one of the preceding claims, or a pharmaceutically acceptable salt thereof, wherein R₃ is cyano, or a group of the formula 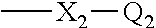
wherein:
X₂ is absent or is selected from the group consisting of —O—, —C(O)—, —NR₃ₐ—, —C(O)—O—, —O—C(O)—, —S(O)₀₋₂—, —C(O)—N(R₃ₐ)—, —N(R₃ₐ)—C(O)—, —N(R_{3b})—C(O)—O—, —N(R_{3b})—C(O)—NR₃ₐ—, —SO₂N(R₃ₐ)— or —N(R₃ₐ)SO₂—, where R₃ₐ is selected from the group consisting of hydrogen or (1-2C)alkyl;
Q₂ is selected from the group consisting of hydrogen, (1-4C)alkyl, (3-6C)cycloalkyl, (3-6C)cycloalkyl(1-2C)alkyl, phenyl, phenyl(1-4C)alkyl, 4- to 6-membered heterocyclyl, [4- to 6-membered heterocyclyl](1-2C)alkyl, 5- to 10-membered heteroaryl and [5- to 10-membered heteroaryl](1-2C)alkyl, wherein any alkyl, cycloalkyl, cycloalkyl-alkyl, phenyl, phenylalkyl, heterocyclyl, heterocyclyl-alkyl, heteroaryl or heteroaryl-alkyl in Q₂ is optionally substituted with one or more R₃, groups; and
each R₃, group present is independently selected from the group consisting of hydroxy, cyano, oxo, halogen, (1-4C)alkyl, (1-4C)alkoxy, —NR_{3d}R₃ₑ, —C(O)—R_{3d}, —S(O)₀₋₂R_{3d}—, —S(O)₀₋₂R_{3d}—, phenyl, 5 or 6-membered heteroaryl, or 4 to 6-membered heterocyclyl,
wherein R_{3d} and R₃ₑ are each independently hydrogen or (1-2C)alkyl;
and wherein any alkyl, alkoxy, phenyl, heteroaryl or heterocyclyl group present in a R₃ₑ group, is optionally further substituted by one or more substituents independently selected from hydroxy, cyano, halogen, (1-2C)alkyl, or —OR_{3f}, wherein R_{3f} is selected from hydrogen and (1-2C)alkyl.

9. A compound according to any one of the preceding claims, or a pharmaceutically acceptable salt thereof, wherein R₃ is cyano, or a group of the formula 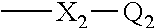
wherein:
X₂ is absent or is selected from the group consisting of —O—, —C(O)—, —NR₃ₐ—, —C(O)—O—, —S(O)₀₋₂—, —C(O)—N(R₃ₐ)—, —N(R₃ₐ)—C(O)—, —N(R_{3b})—C(O)—O—, —N(R_{3b})—C(O)—NR₃ₐ—, —SO₂N(R₃ₐ)— or —N(R₃,)SO₂—, where R₃ₐ is selected from the group consisting of hydrogen or methyl;
Q₂ is selected from the group consisting of hydrogen, (1-3C)alkyl, (3-6C)cycloalkyl, (3-6C)cycloalkyl(1-2C)alkyl, phenyl, phenyl(1-4C)alkyl, 4- to 6-membered heterocyclyl, [4- to 6-membered heterocyclyl](1-2C)alkyl, 5- to 10-membered heteroaryl and [5- to 10-membered heteroaryl](1-2C)alkyl, wherein any alkyl, cycloalkyl, cycloalkyl-alkyl, phenyl, phenylalkyl, heterocyclyl, heterocyclyl-alkyl, heteroaryl or heteroaryl-alkyl in Q₂ is optionally substituted with one or more R₃, groups; and
each R₃, group present is independently selected from the group consisting of hydroxy, cyano, oxo, halogen, (1-4C)alkyl, (1-4C)alkoxy, —C(O)—R_{3d}, —C(O)—OR_{3d}, —O—C(O)—R_{3d}, —C(O)—NR_{3d}R₃ₑ, —N(R₃ₑ)C(O)—R_{3d}, —S(O)₀₋₂R_{3d}—, phenyl, 5 or 6-membered heteroaryl, or 4 to 6-membered heterocyclyl, wherein R_{3d} and R₃ₑ are each independently hydrogen or (1-2C)alkyl;
and wherein any alkyl, alkoxy, phenyl, heteroaryl or heterocyclyl group present in a R₃ₑ group, is optionally further substituted by one or more substituents independently selected from hydroxy or halogen.

10. A compound according to any one of the preceding claims, or a pharmaceutically acceptable salt thereof, wherein R₃ is cyano, or a group of the formula 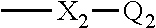
wherein:
X₂ is absent or is selected from the group consisting of —O—, —NR₃ₐ—, —C(O)—O—, —C(O)—N(R₃ₐ)—, —N(R₃ₐ)—C(O)—, —N(R_{3b})—C(O)—NR₃ₐ— or —N(R₃ₐ)SO₂—, where R₃ₐ is selected from the group consisting of hydrogen or methyl;
Q₂ is selected from the group consisting of hydrogen, (1-3C)alkyl, (3-6C)cycloalkyl, (3-6C)cycloalkyl(1-2C)alkyl, phenyl, phenyl(1-4C)alkyl, 4- to 6-membered heterocyclyl, [4- to 6-membered heterocyclyl](1-2C)alkyl, 5- to 10-membered heteroaryl and [5- to 10-membered heteroaryl](1-2C)alkyl, wherein any alkyl, cycloalkyl, cycloalkyl-alkyl, phenyl, phenylalkyl, heterocyclyl, heterocyclyl-alkyl, heteroaryl or heteroaryl-alkyl in Q₂ is optionally substituted with one or more R₃, groups; and
each R_{3c} group present is independently selected from the group consisting of hydroxy, cyano, oxo, halogen, (1-4C)alkyl, (1-4C)alkoxy, —C(O)—R_{3d}, —S(O)₀₋₂R_{3d}—, or 5 or 6-membered heteroaryl;
wherein R_{3d} and R₃ₑ are each independently hydrogen or (1-2C)alkyl;
and wherein any alkyl, alkoxy, phenyl, heteroaryl or heterocyclyl group present in a R₃, group, is optionally further substituted by one or more substituents independently selected from hydroxy or halogen.

11. A compound according to any one of the preceding claims, or a pharmaceutically acceptable salt thereof, wherein R₃ is a group of the formula: 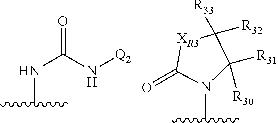
wherein:
Q₂ is selected from hydrogen, (1-6C)alkyl, (3-6C)cycloalkyl, (3-6C)cycloalkyl(1-2C)alkyl, 4- to 6-membered heterocyclyl or [4- to 6-membered heterocyclyl](1-2C)alkyl, each of which is optionally substituted by one or more R_{K}c substituents;
X_{R3} is O, NH or N(1-2C)alkyl;
R₃₀, R₃₁, R₃₂ and R₃₃ are each independently selected from hydrogen, (1-6C)alkyl, (3-6C)cycloalkyl, (3-6C)cycloalkyl(1-2C)alkyl, 4- to 6-membered heterocyclyl or [4- to 6-membered heterocyclyl](1-2C)alkyl, and wherein any alkyl, cycloalkyl, cycloalkyl-alkyl, heterocyclyl and heterocyclyl-alkyl is optionally substituted with one or more R_{3c} substituents;
or either R₃₀ and R₃₁ or R₃₂ and R₃₃ are linked such that, together with the carbon atom to which they are attached, they form a spiro fused 3 to 6 membered cycloalkyl or heterocyclyl ring, which is optionally substituted with one or more R_{3c} substituents;
wherein each R₃ₑ present is independently selected from hydroxy, cyano, oxo, halogen, (1-4C)alkyl, (1-4C)alkoxy, (3-6C)cycloalkyl, (3-6C)cycloalkyl(1-2C)alkyl, —NR_{3d}R₃ₑ, —C(O)—R_{3d}, —C(O)—OR_{3d}, —O—C(O)—R_{3d}, —C(O)—NR_{3d}R₃ₑ, —N(R₃ₑ)C(O)—R_{3d}, —S(O)₀₋₂R_{3d}—, —S(O)₂NR_{3d}R₃ₑ, —N(R₃ₑ)—S(O)₂R_{3d};
wherein R_{3d} and R₃ₑ are each independently hydrogen, (1-4C)alkyl, (3-6C)cycloalkyl, or (3-6C)cycloalkyl(1-2C)alkyl

12. A compound according to any one of the preceding claims, or a pharmaceutically acceptable salt thereof, wherein R₃ is a group of the formula:

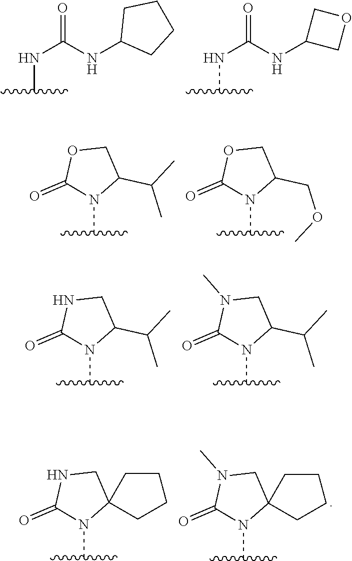

13. A compound according to any one of the preceding claims, or a pharmaceutically acceptable salt thereof, wherein:
(1) Q₁ is selected from:
(i) a group of the formula: 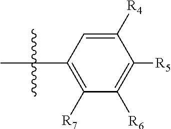
wherein:
R₄, R₆ and R₇ are each independently selected from hydrogen, halo, cyano, or a group of the formula: 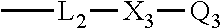
wherein:
L₂ is absent or (1-3C)alkylene;
X₃ is absent or is selected from the group consisting of —O—, —NR₄ₐ—, —C(O)—O—, —O—C(O)—, —S(O)₀₋₂—, —C(O)—N(R₄ₐ)—, —N(R₄ₐ)—C(O)—, —SO₂N(R₄ₐ)— or —N(R₄ₐ)SO₂—, where R₄ₐ and R_{4b} are independently selected from the group consisting of hydrogen or (1-2C)alkyl;
Q₃ is selected from the group consisting of hydrogen, (1-4C)alkyl, (3-6C)cycloalkyl, (3-6C)cycloalkyl(1-2C)alkyl, phenyl, phenyl(1-2C)alkyl, 3 to 8-membered heterocyclyl, [3 to 8-membered heterocyclyl](1-2C)alkyl, 5 or 6-membered heteroaryl and 5 or 6-membered heteroaryl(1-2C)alkyl, wherein any alkyl, cycloalkyl, cycloalkylalkyl, aryl, arylalkyl, heterocyclyl, heterocyclyl-alkyl, heteroaryl or heteroaryl-alkyl in Q₃ is optionally substituted with one or more R₄ₑ groups; and
each R_{4c} group present is independently selected from the group consisting of hydroxy, cyano, oxo, halogen, (1-4C)alkyl, (1-4C)alkoxy, (3-6C)cycloalkyl, (3-6C)cycloalkyl(1-2C)alkyl, —NR_{4d}R₄ₑ, —C(O)—R_{4d}, —C(O)—OR_{4d}, —O—C(O)—R_{4d}, —C(O)—NR_{4d}R₄ₑ, —N(R₄ₑ)C(O)—R_{4d}, —S(O)₀₋₂R_{4d}—, —S(O)₂NR_{4d}R₄ₑ, or —N(R₄ₑ)—S(O)₂R_{4d},
wherein R_{4d} and R₄ₑ are each independently hydrogen or (1-4C)alkyl; and wherein any alkyl, alkoxy, cycloalkyl, cycloalkyl-alkyl group present in a R₄ₑ group, or any alkyl group present in a R_{4d} or R₄ₑ group, is optionally further substituted by one or more substituents independently selected from hydroxy, cyano, halogen, R_{4f}, —OR_{4f}, —NR_{4f}R_{4g}—C(O)OR_{4f}, —OC(O)R_{4f}, —C(O)NR_{4f}R_{4g}, —NR_{4g}C(O)R_{4f}, —S(O)O-2—R_{4f} and —C(O)—R_{4f}, wherein R_{4f} and R_{4g} are both independently selected from hydrogen or (1-2C)alkyl;
R₅ is:
a) hydrogen, halo, cyano; or
b) a group of the formula: 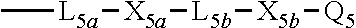
wherein:
L₅ₐ is absent or (1-3C)alkylene;
X₅ₐ is absent or is selected from the group consisting of —O—, —C(O)—, —NR₅ₐ₁—, —C(O)—O—, —O—C(O)—, —S(O)₀₋₂—, —C(O)—N(R₅ₐ₁)—, —N(R₅ₐ₁)—C(O)—, —SO₂N(R₅ₐ₁)— or —N(R₅ₐ₁)SO₂—, where R₅ₐ₁ and R₅ₐ₂ are independently selected from the group consisting of hydrogen or (1-4C)alkyl;
L_{5b} is absent or (1-4C)alkylene;
X_{5b} is absent or is selected from the group consisting of —O—, —C(O)—, —NR_{5b1}—, —C(O)—O—, —O—C(O)—, —S(O)₀₋₂—, —C(O)—N(R_{5b1})—, —N(R_{5b1})—C(O)—, —O—C(O)—N(R_{5b1})—, —N(R_{5b1})—C(O)—O—, —N(R_{5b2})—C(O)—NR_{5b1}—, —SO₂N(R_{5b1})— or —N(R_{5b1})SO₂—, where R_{5b1} and R_{5b2} are independently selected from the group consisting of hydrogen or (1-4C)alkyl;
Q₅ is selected from the group consisting of hydrogen, (1-6C)alkyl, (3-8C)cycloalkyl, (3-8C)cycloalkyl(1-4C)alkyl, phenyl, phenyl(1-2C)alkyl, 3 to 8-membered heterocyclyl, [3 to 8-membered heterocyclyl](1-2C)alkyl, 5 or 6-membered heteroaryl and 5 or 6-membered heteroaryl(1-2C)alkyl and wherein Q₅ is optionally substituted with one or more R_{5c} groups;
and each R_{5c} group present is independently selected from the group consisting of hydroxy, cyano, oxo, halogen, (1-6C)alkyl, (1-6C)alkoxy, (3-8C)cycloalkyl (including spiro-fused (3-8C)cycloalkyl), or (3-8C)cycloalkyl(1-2C)alkyl, —NR_{5d}R₅ₑ, —C(O)—R_{5d}, —C(O)—OR_{5d}, —O—C(O)—R_{5d}, —C(O)—NR_{5d}R₅ₑ, —N(R₅ₑ)C(O)—R_{5d}, —S(O)₀₋₂R_{5d}—, —S(O)₂NR_{5d}R₅ₑ, —N(R₅ₑ)—S(O)₂R_{5d}, phenyl, 5 or 6-membered heteroaryl, or 4 to 8-membered heterocyclyl,
wherein R_{5d} and R₅ₑ are each independently hydrogen, (1-6C)alkyl, (3-6C)cycloalkyl, or (3-6C)cycloalkyl(1-2C)alkyl;
and wherein any alkyl, alkoxy, cycloalkyl, cycloalkyl-alkyl, phenyl, heteroaryl or heterocyclyl group present in a R_{5c} group, or any alkyl, cycloalkyl or cycloalkyl-alkyl group present in a R_{5d} or R₅ₑ group, is optionally further substituted by one or more substituents independently selected from hydroxy, cyano, halo, R_{5f}, —OR_{5f}, —NR_{5f}R_{5g} and —C(O)—R_{5f}, wherein R_{5f} and R₅ₑ are both independently selected from hydrogen, (1-4C)alkyl, hydroxy(1-4C)alkyl, halo(1-4C)alkyl, or (1-2C)alkoxy(1-4C)alkyl;
or R₄ and R₅, R₅ and R₆, or R₆ and R₇ are linked to form a fused phenyl, 5- or 6-membered heteroaryl or 5 to 7-membered heterocyclic ring, which is optionally substituted by one or more substituent groups selected from hydroxy, cyano, halo, (1-2C)alkyl, (1-2C)haloalkyl, (1-2C)alkoxy or (1-2C)haloalkoxy;
(ii) a group of the formula: 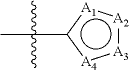
wherein:
A₁ is selected from N, NR_{1N}, O, S or CR₇;
A₂ is selected from N, NR_{1N}, O, S or CR₄;
A₃ is selected from N, NR_{1N}, O, S or CR₅;
A₄ is selected from N, NR_{1N}, O, S or CR₇;
R_{1N} is selected from hydrogen, (1-4C)alkyl, (2-4C)alkanoyl, phenyl, benzyl, (3-8C)cycloalkyl, (3-8C)cycloalkyl-(1-2C)alkyl, a carbon-linked 4 to 7 membered heterocyclyl, or 4 to 7 membered heterocyclyl(1-2C)alkyl, and wherein any alkyl, alkanoyl, cycloalkyl, cycloalkyl-alkyl, phenyl, benzyl, heterocyclyl, heterocyclyl-alkyl group is optionally substituted with one or more R_{5c} groups defined above;
R₄, R₅ and R₇ are as defined above;
and with the proviso that:
(i) only one of A₁, A₂, A₃ and A₄ can be O or S;
(ii) only one of A₁, A₂, A₃ and A₄ can be NR_{1N};
(iii) 1 to 3 of A₁, A₂, A₃ and A₄ can be N;
(iii) a group of the formula: 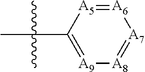
wherein:
A₉ is selected from N or CH;
A₆ is selected from N or CR₄;
A₇ is selected from N or CR₅;
A₉ is selected from N or CR₆;
A₉ is selected from N or CR₇;
R₄, R₅, R₆ and R₇ are as defined above;
and with the proviso that only one of A₅, A₆, A₇, A₈ and A₉ can be N;
(iv) a group of the formula: 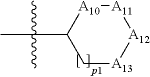
wherein:
p1 is 0 or 1;
A₁₀ is selected from NH, CH₂ or CHR₇;
A₁₁ is selected from NR_{4N}, CH₂ or CHR₄;
A₁₂ is selected from NR_{5N}, CH₂ or CHR₅;
A₁₃ is selected from NR_{6N}, CH₂ or CHR₆;
R₄, R₅, R₆ and R₇ are as defined above;
R_{4N} and R_{6N} are each independently selected from hydrogen or a group of the formula: 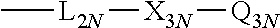
wherein:
L_{2N} is absent or (1-3C)alkylene;
X_{3N} is absent or is selected from the group consisting of:
(i) —C(O)—, —S(O)₀₋₂— or —C(O)—N(R₄ₐ)— when L₂ is absent; or
(ii) —O—, —C(O)—, —NR₄ₐ—, —C(O)—O—, —O—C(O)—, —S(O)₀₋₂—, —C(O)—N(R₄ₐ)—, —N(R₄ₐ)—C(O)—, —O—C(O)—N(R₄ₐ)—, —N(R₄)—C(O)—O—, —N(R_{4b})—C(O)—NR₄ₐ—, —SO₂N(R₄ₐ)— or —N(R₄ₐ)SO₂— when L₂ is (1-3C)alkylene;
wherein R₄ₐ and R_{4b} are as defined above;
Q_{3N} is selected from the group consisting of hydrogen, (1-6C)alkyl, (3-6C)cycloalkyl, (3-6C)cycloalkyl(1-2C)alkyl, heterocyclyl, heterocyclyl(1-2C)alkyl, heteroaryl and heteroaryl(1-2C)alkyl, wherein any alkyl, cycloalkyl, cycloalkylalkyl, heterocyclyl, heterocyclylalkyl, heteroaryl or heteroarylalkyl in Q₃ is optionally substituted with one or more R₄ₑ groups; and
each R₄ₑ group present is as defined above;
R_{5N} is:
a) hydrogen;
b) a group of the formula: 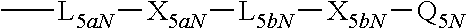
wherein:
L_{5aN} is absent or (1-3C)alkylene;
X_{5aN} is absent or is selected from the group consisting of:
(i) —C(O)—, —S(O)₀₋₂—, —C(O)—N(R₅ₐ₁)— when L_{5aN} is absent; or
(ii) —O—, —C(O)—, —NR₅ₐ₁—, —C(O)—O—, —O—C(O)—, —S(O)₀₋₂—, —C(O)—N(R₅ₐ₁)—, —N(R₅ₐ₁)—C(O)—, —O—C(O)—N(R₅ₐ₁)—, —N(R₅ₐ₁)—C(O)—O—, —N(R₅ₐ₂)—C(O)—NR₅ₐ₁—, —SO₂N(R₅ₐ) or —N(R₅ₐ₁)SO₂— when L_{5aN} is present;
where R₅,1 and R₅ₐ₂ are as defined above;
L_{5bN} is absent or (1-2C)alkylene;
X_{5bN} is absent or is selected from the group consisting of —O—, —C(O)—, —NR_{5b1}—, —C(O)—O—, —O—C(O)—, —S(O)₀₋₂—, —C(O)—N(R_{5b1})—, —N(R_{5b1})—C(O)—, —SO₂N(R_{5b1})— or —N(R_{5b1})SO₂—, where R_{5b1} and R_{5b2} are independently selected from the group consisting of hydrogen or (1-4C)alkyl;
Q_{5N} is selected from the group consisting of:
(i) hydrogen;
(ii) (1-4C)alkyl, (3-6C)cycloalkyl, (3-6C)cycloalkyl(1-1C)alkyl, phenyl, phenyl(1-2C)alkyl, 5- or 6-membered heteroaryl, 5- or 6-membered heteroaryl(1-2C)alkyl;
(iii) a carbon-linked 4 to 8 membered heterocyclyl (when -L_{5aN}, X_{5aN}, L_{5bN}, and X_{5bN} are absent) or a 4 to 8 membered heterocyclyl (when one or more of -L_{5aN}, X_{5aN}, L_{5bN}, and X_{5bN} are present), or [4 to 8 membered heterocyclyl](1-2C)alkyl;
and wherein Q_{5N} is optionally substituted with one or more R_{5c} groups as defined above;
and with the proviso that one or two of A₁₀, A₁₁, A₁₂ and A₁₃ can be N;
(v) a group of the formula: 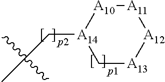
wherein:
p1, A₁₀, A₁₁, A₁₂, and A₁₃ are as defined above;
p2 is 1 to 5;
A₁₄ is C or N;
and with the proviso that one of A₁₀, A₁₁, A₁₂ and A₁₃ can be N.
(2) Q₁ is selected from:
(i) a group of the formula: 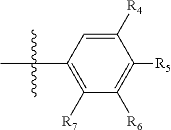
wherein:
R₄, R₆ and R₇ are each independently selected from hydrogen, or a group of the formula: 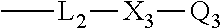
wherein:
L₂ is absent or (1-3C)alkylene;
X₃ is absent or is selected from the group consisting of —O—, —NR₄ₐ—, —S(O)₀₋₂—, —C(O)—N(R₄ₐ)—, —N(R₄ₐ)—C(O)—, —SO₂N(R₄ₐ)— or —N(R₄ₐ)SO₂—, where R₄ₐ and R_{4b} are independently selected from the group consisting of hydrogen or (1-2C)alkyl;
Q₃ is selected from the group consisting of hydrogen, (1-4C)alkyl, (3-6C)cycloalkyl, (3-8C)cycloalkyl(1-4C)alkyl, phenyl, 3 to 8-membered heterocyclyl, 5 or 6-membered heteroaryl or 5 or 6-membered heteroaryl(1-4C)alkyl wherein any alkyl, cycloalkyl, cycloalkylalkyl, phenyl, heterocyclyl, or heteroaryl in Q₃ is optionally substituted with one or more R_{4c} groups; and
each R_{4c} group present is independently selected from the group consisting of hydroxy, cyano, oxo, halogen, (1-4C)alkyl, (1-4C)alkoxy, (3-6C)cycloalkyl, (3-6C)cycloalkyl(1-2C)alkyl, —NR_{4d}R₄ₑ, —C(O)—R_{4d}, —C(O)—OR_{4d}, —O—C(O)—R_{4d}, —C(O)—NR_{4d}R₄ₑ, —N(R₄ₑ)C(O)—R_{4d}, —S(O)₀₋₂R_{4d}—, —S(O)₂NR_{4d}R₄ₑ, or —N(R₄ₑ)—S(O)₂R_{4d},
wherein R_{4d} and R₄ₑ are each independently hydrogen or (1-4C)alkyl;
and wherein any alkyl, alkoxy, cycloalkyl, cycloalkyl-alkyl, phenyl, heteroaryl or heterocyclyl group present in a R_{4c} group, or any alkyl group present in a R_{4d} or R₄ₑ group, is optionally further substituted by one or more substituents independently selected from hydroxy, cyano, halogen, (1-2C)alkyl, —OR_{4f}, —NR_{4f}R_{4g}, —S(O)₀₋₂—R_{4f} and —C(O)—R_{4f}, wherein R_{4f} and R_{4g} are both independently selected from hydrogen and (1-2C)alkyl;
R₅ is:
a) hydrogen, halo, cyano; or
b) a group of the formula: 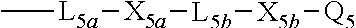
wherein:
L₅ₐ is absent or (1-3C)alkylene;
X₅ₐ is absent or is selected from the group consisting of —O—, —C(O)—, —NR₅ₐ₁—, —S(O)₀₋₂—, —C(O)—N(R₅ₐ₁)—, —N(R₅ₐ₁)—C(O)—, —SO₂N(R₅ₐ₁)— or —N(R₅ₐ₁)SO₂—, where R₅ₐ₁ and R₅ₐ₂ are independently selected from the group consisting of hydrogen or (1-2C)alkyl;
L_{5b} is absent or (1-4C)alkylene;
X_{5b} is absent or is selected from the group consisting of —O—, —C(O)—, —NR_{5b}—, —S(O)₀₋₂—, —C(O)—N(R_{5b1})—, —N(R_{5b1})—C(O)—, —SO₂N(R_{5b1})— or —N(Rbl)SO₂—, where R_{5b1} and R_{5b2} are independently selected from the group consisting of hydrogen or (1-2C)alkyl;
Q₅ is selected from the group consisting of hydrogen, (1-6C)alkyl, (3-8C)cycloalkyl, phenyl, 3 to 8-membered heterocyclyl, or 5 or 6-membered heteroaryl and wherein Q₅ is optionally substituted with one or more R_{5c} groups;
and each R_{5c} group present is independently selected from the group consisting of hydroxy, cyano, halogen, (1-6C)alkyl, (1-6C)alkoxy, (3-8C)cycloalkyl (including spiro-fused (3-8C)cycloalkyl), (3-8C)cycloalkyl(1-2C)alkyl, —NR_{5d}R₅ₑ, —C(O)—R_{5d}, —C(O)—NR_{5d}R₅ₑ, —N(R₅ₑ)C(O)—R_{5d}, —S(O)₀₋₂R_{5d}—, —S(O)₂NR_{5d}R₅ₑ, —N(R₅ₑ)—S(O)₂R_{5d}, phenyl, 5 or 6-membered heteroaryl, or 4 to 8-membered heterocyclyl,
wherein R_{5d} and R₅ₑ are each independently hydrogen, (1-6C)alkyl, (3-6C)cycloalkyl, or (3-6C)cycloalkyl(1-2C)alkyl;
and wherein any alkyl, alkoxy, cycloalkyl, cycloalkyl-alkyl, phenyl, heteroaryl or heterocyclyl group present in a R_{5c} group, or any alkyl, cycloalkyl or cycloalkyl-alkyl group present in a R_{5d} or R₅ₑ group, is optionally further substituted by one or more substituents independently selected from hydroxy, cyano, halo, R_{5f}, —OR_{5f}, —NR_{5f}R_{5g} and —C(O)—R_{5f}, wherein R_{5f} and R_{5g} are both independently selected from hydrogen, (1-4C)alkyl, hydroxy(1-4C)alkyl, halo(1-4C)alkyl, or (1-2C)alkoxy(1-4C)alkyl;
or R₄ and R₅, R₅ and R₆, or R₆ and R₇ are linked to form a fused phenyl, 5- or 6-membered heteroaryl or 5 to 7-membered heterocyclic ring, which is optionally substituted by one or more substituent groups selected from hydroxy, cyano, halo, (1-2C)alkyl, (1-2C)haloalkyl, (1-2C)alkoxy or (1-2C)haloalkoxy;
(ii) a group of the formula: 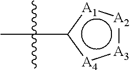
wherein:
A₁ is selected from N, NR_{1N}, O, S or CR₇;
A₂ is selected from N, NR_{1N}, O, S or CR₄;
A₃ is selected from N, NR_{1N}, O, S or CR₅;
A₄ is selected from N, NR_{1N}, O, S or CR₇;
R_{1N} is selected from hydrogen, (1-4C)alkyl, (2-4C)alkanoyl, phenyl, benzyl, (3-8C)cycloalkyl, (3-8C)cycloalkyl-(1-2C)alkyl, a carbon-linked 4 to 7 membered heterocyclyl, or 4 to 7 membered heterocyclyl(1-2C)alkyl, and wherein any alkyl, alkanoyl, cycloalkyl, cycloalkyl-alkyl, phenyl, benzyl, heterocyclyl, heterocyclyl-alkyl group is optionally substituted with one or more R_{5C} groups defined above;
R₄, R₅ and R₇ are as defined above;
and with the proviso that:
(i) only one of A₁, A₂, A₃ and A₄ can be O or S;
(ii) only one of A₁, A₂, A₃ and A₄ can be NR_{1N};
(iii) 1 to 3 of A₁, A₂, A₃ and A₄ can be N;
(iii) a group of the formula: 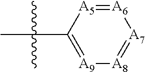
wherein:
A₅ is selected from N or CH;
A₉ is selected from N or CR₄;
A₇ is selected from N or CR₅;
A₈ is selected from N or CR₅;
A₉ is selected from N or CR₇;
R₄, R₅, R₆ and R₇ are as defined above;
and with the proviso that only one of A₅, A₆, A₇, A₈ and A₉ can be N;
(iv) a group of the formula: 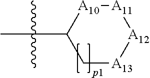
wherein:
p1 is 0 or 1;
A₁₀ is selected from NH, CH₂ or CHR₇;
A₁₁ is selected from NR_{4N}, CH₂ or CHR₄;
A₁₂ is selected from NR_{5N}, CH₂ or CHR₅;
A₁₃ is selected from NR_{6N}, CH₂ or CHRo;
R₄, R₅, R₆ and R₇ are as defined above;
R_{4N} and R_{6N} are each independently selected from hydrogen or a group of the formula: 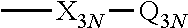
wherein:
X_{3N} is absent or is selected from the group consisting of: —C(O)—, —S(O)₀₋₂— or —C(O)—N(R₄ₐ)—;
wherein R₄ₐ is as defined above;
Q_{3N} is selected from the group consisting of hydrogen, (1-4C)alkyl, (3-6C)cycloalkyl, (3-6C)cycloalkyl(1-2C)alkyl, heterocyclyl, heteroaryl and, wherein any alkyl, cycloalkyl, cycloalkylalkyl, heterocyclyl or heteroaryl in Q₃ is optionally substituted with one or more R₄ₑ groups; and
each R₄, group present is as defined above;
R_{5N} is:
a) hydrogen;
b) a group of the formula: 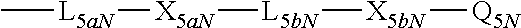
wherein:
L_{5aN} is absent or (1-3C)alkylene;
X_{5aN} is absent or is selected from the group consisting of:
(i) —C(O)—, —S(O)₀₋₂—, —C(O)—N(R₅ₐ₁)— when L_{5aN} is absent; or
(ii) —O—, —C(O)—, —NR₅ₐ₁—, —C(O)—O—, —O—C(O)—, —S(O)₀₋₂—, —C(O)—N(R₅ₐ₁)—, —N(R₅ₐ₁)—C(O)—, —O—C(O)—N(R₅ₐ₁)—, —N(R₅ₐ₁)—C(O)—O—, —N(R₅ₐ₂)—C(O)—NR₅ₐ₁—, —SO₂N(R₅ₐ₁)— or —N(R₅ₐ₁)SO₂— when L_{5aN} is present;
where R₅ₐ₁ and R₅ₐ₂ are as defined above;
L_{5bN} is absent or (1-20)alkylene;
X_{5bN} is absent or is selected from the group consisting of —O—, —C(O)—, —NR_{5b1}—, —S(O)₀₋₂—, —C(O)—N(R_{5b1})—, —N(R_{5b1})—C(O)—, —SO₂N(R_{5b1})— or —N(R_{5b1})SO₂—, where R_{5b1} and R_{5b2} are independently selected from the group consisting of hydrogen or (1-2C)alkyl;
Q_{5N} is selected from the group consisting of:
(i) hydrogen;
(ii) (1-4C)alkyl, (3-6C)cycloalkyl, (3-6C)cycloalkyl(1-1C)alkyl, phenyl, phenyl(1-2C)alkyl, 5- or 6-membered heteroaryl, 5- or 6-membered heteroaryl(1-2C)alkyl;
and wherein Q_{5N} is optionally substituted with one or more R_{5c} groups as defined above;
and with the proviso that one or two of A₁₀, A₁₁, A₁₂ and A₁₃ can be N;
(v) a group of the formula: 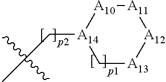
wherein:
p1, A₁₀, A₁₁, A₁₂, and A₁₃ are as defined above;
p2 is 1 to 4;
A₁₄ is C or N;
and with the proviso that one of A₁₀, A₁₁, A₁₂ and A₁₃ can be N.
(3) Q, is selected from:
(i) a group of the formula: 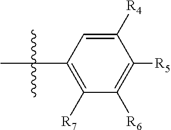
wherein:
R₄, R₆ and R₇ are each independently selected from hydrogen, or a group of the formula: 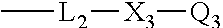
wherein:
L₂ is absent or (1-2C)alkylene;
X₃ is absent or is selected from the group consisting of —O—, —NR₄ₐ—, —C(O)—N(R₄ₐ)—, —N(R₄ₐ)—C(O)—, —SO₂N(R₄ₐ)— or —N(R₄ₐ)SO₂—, where R₄ₐ and R_{4b} are independently selected from the group consisting of hydrogen or (1-2C)alkyl;
Q₃ is selected from the group consisting of hydrogen, (1-4C)alkyl, (3-6C)cycloalkyl, (3-6C)cycloalkyl(1-4C)alkyl, phenyl, 3 to 8-membered heterocyclyl, 5 or 6-membered heteroaryl or 5 or 6-membered heteroaryl(1-4C)alkyl wherein any alkyl, cycloalkyl, cycloalkylalkyl, phenyl, heterocyclyl or heteroaryl in Q₃ is optionally substituted with one or more R₄ₑ groups; and
each R_{4c} group present is independently selected from the group consisting of hydroxy, cyano, oxo, halogen, (1-4C)alkyl, (1-4C)alkoxy, (3-6C)cycloalkyl, (3-6C)cycloalkyl(1-2C)alkyl, —NR_{4d}R₄ₑ, —C(O)—R_{4d}, —C(O)—OR_{4d}, —O—C(O)—R_{4d}, —C(O)—NR_{4d}R₄ₑ, —N(R₄ₑ)C(O)—R_{4d}, —S(O)₀₋₂R_{4d}—, —S(O)₂NR_{4d}R₄ₑ, or —N(R₄ₑ)—S(O)₂R_{4d},
wherein R_{4d} and R₄ₑ are each independently hydrogen or (1-2C)alkyl;
and wherein any alkyl, alkoxy, cycloalkyl, cycloalkyl-alkyl, phenyl, heteroaryl or heterocyclyl group present in a R_{4c} group, or any alkyl group present in a R_{4d} or R₄ₑ group, is optionally further substituted by one or more substituents independently selected from hydroxy, cyano, halogen, (1-2C)alkyl, —OR_{4f}, —NR_{4f}R_{4g}, —S(O)₀₋₂—R_{4f} and —C(O)—R_{4f}, wherein R_{4f} and R_{4g} are both independently selected from hydrogen and (1-2C)alkyl;
R₅ is:
a) hydrogen, halo, cyano; or
b) a group of the formula: 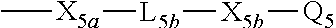
wherein:
X₅ₐ is absent or is selected from the group consisting of —O—, —C(O)—, —NR₅ₐ₁—, —S(O)₀₋₂—, —C(O)—N(R₅ₐ₁)—, —N(R₅ₐ₁)—C(O)—, —SO₂N(R₅ₐ₁)— or —N(R₅ₐ₁)SO₂—, where R₅ₐ₁ and R₅ₐ₂ are independently selected from the group consisting of hydrogen or (1-2C)alkyl;
L_{5b} is absent or (1-4C)alkylene;
X_{5b} is absent or is selected from the group consisting of —O—, —C(O)—, —NR_{5b}—, —S(O)₀₋₂—, —C(O)—N(R_{5b1})—, —N(R_{5b1})—C(O)—, —SO₂N(R_{5b1})— or —N(R_{5b1})SO₂—, where R_{5b1} and R_{5b2} are independently selected from the group consisting of hydrogen or (1-2C)alkyl;
Q₅ is selected from the group consisting of hydrogen, (1-6C)alkyl, (3-8C)cycloalkyl, phenyl, 3 to 8-membered heterocyclyl, or 5 or 6-membered heteroaryl and wherein Q₅ is optionally substituted with one or more R_{5c} groups;
and each R_{5c} group present is independently selected from the group consisting of hydroxy, cyano, halogen, (1-6C)alkyl, (1-6C)alkoxy, (3-8C)cycloalkyl (including spiro-fused (3-8C)cycloalkyl), (3-8C)cycloalkyl(1-2C)alkyl, —NR_{5d}R₅ₑ, —C(O)—R_{5d}, —C(O)—NR_{5d}R₅ₑ, —N(R₅ₑ)C(O)—R_{5d}, —S(O)₀₋₂R_{5d}—, —S(O)₂NR_{5d}R₅ₑ, —N(R₅ₑ)—S(O)₂R_{5d}, phenyl, 5 or 6-membered heteroaryl, or 4 to 8-membered heterocyclyl,
wherein R_{5d} and R₅ₑ are each independently hydrogen, (1-6C)alkyl, (3-6C)cycloalkyl, or (3-6C)cycloalkyl(1-2C)alkyl;
and wherein any alkyl, alkoxy, cycloalkyl, cycloalkyl-alkyl, phenyl, heteroaryl or heterocyclyl group present in a R_{5c} group, or any alkyl, cycloalkyl or cycloalkyl-alkyl group present in a R_{5d} or R₅ₑ group, is optionally further substituted by one or more substituents independently selected from hydroxy, cyano, halo, R_{5f}, —OR_{5f}, —NR_{5f}R_{5g} and —C(O)—Rsr, wherein R_{5f} and R_{5g} are both independently selected from hydrogen, (1-4C)alkyl, hydroxy(1-4C)alkyl, halo(1-4C)alkyl, or (1-2C)alkoxy(1-4C)alkyl;
or R₄ and R₅, R₅ and R₆, or R₆ and R₇ are linked to form a fused phenyl, 5- or 6-membered heteroaryl or 5 to 7-membered heterocyclic ring, which is optionally substituted by one or more substituent groups selected from hydroxy, cyano, halo, (1-2C)alkyl, (1-2C)haloalkyl, (1-2C)alkoxy or (1-2C)haloalkoxy;
(ii) a group of the formula: 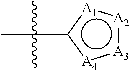
wherein:
A₁ is selected from N, NR_{1N}, O, S or CR₇;
A₂ is selected from N, NR_{1N}, O, S or CR₄;
A₃ is selected from N, NR_{1N}, O, S or CR₅;
A₄ is selected from N, NR_{1N}, O, S or CR₇;
R_{1N} is selected from hydrogen, (1-4C)alkyl, (2-4C)alkanoyl, (3-6C)cycloalkyl, (3-6C)cycloalkyl-(1-2C)alkyl, a carbon-linked 4 to 7 membered heterocyclyl, or 4 to 7 membered heterocyclyl(1-2C)alkyl, and wherein any alkyl, alkanoyl, cycloalkyl, cycloalkyl-alkyl, heterocyclyl, heterocyclyl-alkyl group is optionally substituted with one or more R₅, groups defined above;
R₄, R₅ and R₇ are as defined above;
and with the proviso that:
(i) only one of A₁, A₂, A₃ and A₄ can be O or S;
(ii) only one of A₁, A₂, A₃ and A₄ can be NR_{1N};
(iii) 1 to 3 of A₁, A₂, A₃ and A₄ can be N;
(iii) a group of the formula: 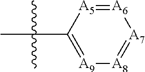
wherein:
A₅ is selected from N or CH;
A₆ is selected from N or CR₄;
A₇ is selected from N or CR₅;
A₈ is selected from N or CR₆;
A₉ is selected from N or CR₇;
R₄, R₅, R₆ and R₇ are as defined above;
and with the proviso that only one of A₅, A₆, A₇, A₈ and A₉ can be N;
(iv) a group of the formula: 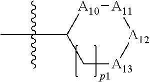
wherein:
p1 is 0 or 1;
A₁₀ is selected from CH₂ or CHR₇;
A₁₁ is selected from CH₂ or CHR₄;
A₁₂ is selected from NR_{5N}, CH₂ or CHR₅;
A₁₃ is selected from CH₂ or CHR₆;
R₄, R₅, R₆ and R₇ are as defined above;
R_{5N} is:
a) hydrogen;
b) a group of the formula: 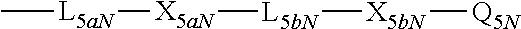
wherein:
L_{5aN} is absent or (1-30)alkylene;
X_{5aN} is absent or is selected from the group consisting of:
(i) —C(O)—, —S(O)₀₋₂—, —C(O)—N(RS₁)— when L_{5aN} is absent; or
(ii) —O—, —C(O)—, —NR₅ₐ₁—, —C(O)—O—, —O—C(O)—, —S(O)O-2—, —C(O)—N(R₅ₐ₁)—, —N(R₅ₐ₁)—C(O)—, —O—C(O)—N(R₅ₐ₁)—, —N(R₅ₐ₁)—C(O)—O—, —N(R₅ₐ₂)—C(O)—NR₅ₐ₁—, —SO₂N(R₅ₐ₁)— or —N(R₅ₐ₁)SO₂— when L_{5aN} is present;
where R₅ₐ₁ and R₅ₐ₂ are as defined above;
L_{5bN} is absent or (1-2C)alkylene;
X_{5bN} is absent or is selected from the group consisting of —O—, —C(O)—, —NR_{5b}—, —S(O)₀₋₂—, —C(O)—N(R_{5b1})—, —N(R_{5b1})—C(O)—, —SO₂N(R_{5b1})— or —N(R_{5b1})SO₂—, where R_{5b1} and R_{5b2} are independently selected from the group consisting of hydrogen or (1-2C)alkyl;
Q_{5N} is selected from the group consisting of:
(i) hydrogen;
(ii) (1-4C)alkyl, (3-6C)cycloalkyl, (3-6C)cycloalkyl(1-1C)alkyl, phenyl, phenyl(1-2C)alkyl, 5- or 6-membered heteroaryl, 5- or 6-membered heteroaryl(1-2C)alkyl;
and wherein Q_{5N} is optionally substituted with one or more R_{5c} groups as defined above;
and with the proviso that one or two of A₁₀, A₁₁, A₁₂ and A₁₃ can be N;
(v) a group of the formula: 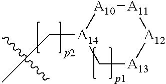
wherein:
p1, A₁₀, A₁₁, A₁₂, and A₁₃ are as defined above;
p2 is 1 to 4;
A₁₄ is C or N;
and with the proviso that one of A₁₀, A₁₁, A₁₂ and A₁₃ can be N.
(4) Q, is selected from:
(i) a group of the formula: 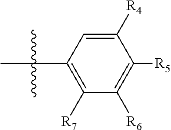
wherein:
R₄, R₆ and R₇ are each independently selected from hydrogen, or a group of the formula: 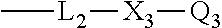
wherein:
L₂ is absent or (1-2C)alkylene;
X₃ is absent or is selected from the group consisting of —O—, —NR₄ₐ—, —C(O)—N(R₄ₐ)—, —N(R₄ₐ)—C(O)—, —SO₂N(R₄ₐ)— or —N(R₄ₐ)SO₂—, where R₄ₐ and R_{4b} are independently selected from the group consisting of hydrogen or methyl;
Q₃ is selected from the group consisting of hydrogen, (1-4C)alkyl, (3-6C)cycloalkyl, (3-6C)cycloalkyl(1-4C)alkyl, phenyl, 3 to 8-membered heterocyclyl, 5 or 6-membered heteroaryl or 5 or 6-membered heteroaryl(1-4C)alkyl wherein any alkyl, cycloalkyl, cycloalkylalkyl, phenyl, heterocyclyl or heteroaryl in Q₃ is optionally substituted with one or more R_{4c} groups; and
each R_{4c} group present is independently selected from the group consisting of hydroxy, cyano, oxo, halogen, methyl, methoxy, (3-6C)cycloalkyl, —NR_{4d}R₄ₑ, —C(O)—R_{4d} or —C(O)—OR_{4d},
wherein R_{4d} and R₄ₑ are each independently hydrogen or methyl;
and wherein any alkyl, alkoxy, cycloalkyl, cycloalkyl-alkyl, phenyl, heteroaryl or heterocyclyl group present in a R_{4c} group, or any methyl group present in a R_{4d} or R₄ₑ group, is optionally further substituted by one or more substituents independently selected from hydroxy, cyano, halogen, (1-2C)alkyl, —OR_{4f}, —NR_{4f}R_{4g}, —S(O)₀₋₂—R_{4f} and —C(O)—R_{4f}, wherein R_{4f} and R_{4g} are both independently selected from hydrogen and (1-2C)alkyl;
R₅ is:
a) hydrogen, halo, cyano; or
b) a group of the formula: 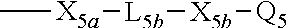
wherein:
X₅ₐ is absent or is selected from the group consisting of —O—, —C(O)—, —NR₅ₐ₁— or —N(R₅ₐ₁)—C(O)—, where R₅ₐ₁ is independently selected from the group consisting of hydrogen or methyl;
L_{5b} is absent or (1-4C)alkylene;
X_{5b} is absent or is selected from the group consisting of —O—, —C(O)— or —NR_{5b1}—, where R_{5b}, and R_{5b2} are independently selected from the group consisting of hydrogen or (1-2C)alkyl;
Q₅ is selected from the group consisting of hydrogen, (1-6C)alkyl or 3 to 8-membered heterocyclyl and wherein Q₅ is optionally substituted with one or more R_{5c} groups;
and each R₅, group present is independently selected from the group consisting of hydroxy, cyano, halogen, methyl, methoxy
(ii) a group of the formula: 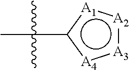
wherein:
A₁ is selected from N, NR_{1N}, O, S or CR₇;
A₂ is selected from N, NR_{1N}, O, S or CR₄;
A₃ is selected from N, NR_{1N}, O, S or CR₅;
A₄ is selected from N, NR_{1N}, O, S or CR₇;
R_{1N} is selected from hydrogen, (1-4C)alkyl, (2-4C)alkanoyl, (3-6C)cycloalkyl, (3-6C)cycloalkyl-(1-2C)alkyl, a carbon-linked 4 to 7 membered heterocyclyl, or 4 to 7 membered heterocyclyl(1-2C)alkyl, and wherein any alkyl, alkanoyl, cycloalkyl, cycloalkyl-alkyl, heterocyclyl, heterocyclyl-alkyl group is optionally substituted with one or more R_{5c} groups defined above;
R₄, R₅ and R₇ are as defined above;
and with the proviso that:
(i) only one of A₁, A₂, A₃ and A₄ can be O or S;
(ii) only one of A₁, A₂, A₃ and A₄ can be NR_{1N};
(iii) 1 to 3 of A₁, A₂, A₃ and A₄ can be N;
(iii) a group of the formula: 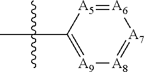
wherein:
A₅ is selected from N or CH;
A₆ is selected from N or CR₄;
A₇ is selected from N or CR₅;
A₈ is selected from N or CR₆;
A₉ is selected from N or CR₇;
R₄, R₅, R₆ and R₇ are as defined above;
and with the proviso that only one of A₅, A₆, A₇, A₈ and A₉ can be N;
(iv) a group of the formula: 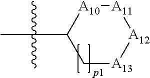
wherein:
p1 is 0 or 1;
A₁₀ is selected from CH₂ or CHR₇;
A₁₁ is selected from CH₂ or CHR₄;
A₁₂ is selected from NR_{5N}, CH₂ or CHR₅;
A₁₃ is selected from CH₂ or CHR₆;
R₄, R₅, R₆ and R₇ are as defined above;
R_{5N} is
a) hydrogen;
b) a group of the formula: 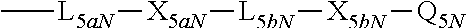
wherein:
L_{5aN} is absent or (1-3C)alkylene;
X_{5aN} is absent or is selected from the group consisting of:
(i) —C(O)—, —S(O)₀₋₂— or —C(O)—N(R₅ₐ₁)— when L_{5aN} is absent; or
(ii) —O—, —C(O)—, —NR₅ₐ₁—, —C(O)—O—, —O—C(O)—, —S(O)₀₋₂—, —C(O)—N(R₅ₐ₁)—, —N(R₅ₐ₁)—C(O)—, —O—C(O)—N(R₅ₐ₁)—, —N(R₅ₐ₁)—C(O)—O— or —N(R₅ₐ₂)—C(O)—NR₅ₐ₁— when L_{5aN} is present;
where R₅ₐ₁ and R₅ₐ₂ are as defined above;
L_{5bN} is absent or (1-2C)alkylene;
X_{5bN} is absent or is selected from the group consisting of —O—, —C(O)—, —NRSb—, —S(O)₀₋₂—, —C(O)—N(R_{5b})— or —N(R_{5b})—C(O)—, where R_{5b}, and R_{5b2} are independently selected from the group consisting of hydrogen or (1-2C)alkyl;
Q_{5N} is selected from the group consisting of:
(i) hydrogen;
(ii) (1-4C)alkyl or (3-6C)cycloalkyl;
and wherein Q_{5N} is optionally substituted with one or more R_{5c} groups as defined above;
and with the proviso that one or two of A₁₀, A₁₁, A₁₂ and A₁₃ can be N;
(v) a group of the formula: 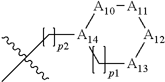
wherein:
p1, A₁₀, A₁₁, A₁₂, and A₁₃ are as defined above;
p2 is 1 to 4;
A₁₄ is C or N;
and with the proviso that one of A₁₀, A₁₁, A₁₂ and A₁₃ can be N.
(5) Q₁ is selected from:
(i) a group of the formula: 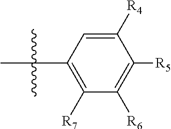
wherein:
two of R₄, R₆ and R₇ are hydrogen, and the other is hydrogen or a group of the formula: 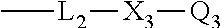
wherein:
L₂ is absent or (1-2C)alkylene;
X₃ is absent or is selected from the group consisting of —O—, —NR₄ₐ—, —C(O)—N(R₄ₐ)—, —N(R₄ₐ)—C(O)—, —SO₂N(R₄ₐ)— or —N(R₄ₐ)SO₂—, where R₄ₐ and R_{4b} are independently selected from the group consisting of hydrogen or methyl;
Q₃ is selected from the group consisting of hydrogen, (1-4C)alkyl, (3-6C)cycloalkyl, (3-6C)cycloalkyl(1-4C)alkyl, phenyl, 3 to 8-membered heterocyclyl, 5 or 6-membered heteroaryl or 5 or 6-membered heteroaryl(1-4C)alkyl wherein any alkyl, cycloalkyl, cycloalkylalkyl, phenyl, heterocyclyl or heteroaryl in Q₃ is optionally substituted with one or more R₄ₑ groups; and
each R_{4c} group present is independently selected from the group consisting of hydroxy, cyano, oxo, halogen, methyl, methoxy, (3-6C)cycloalkyl, —NR_{4d}R₄ₑ, —C(O)—R_{4d} or —C(O)—OR_{4d},
wherein R_{4d} and R₄ₑ are each independently hydrogen or methyl;
and wherein any methyl, methoxy or cycloalkyl, group present in a R₄ₑ group, or any methyl group present in a R_{4d} or R₄ₑ group, is optionally further substituted by one or more substituents independently selected from hydroxy, cyano, halogen, —OR_{4f}, —NR_{4f}R_{4g}, —S(O)₀₋₂—R₄r and —C(O)—R_{4f}, wherein R_{4f} and R_{4g} are both independently selected from hydrogen and (1-2C)alkyl;
R₅ is hydrogen or a group of the formula: 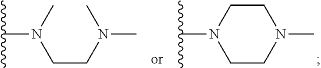
and
(ii) a group of the formula: 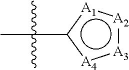
wherein:
A₁ is selected from N, NR_{1N}, O, S or CR₇;
A₂ is selected from N, NR_{1N}, O, S or CR₄;
A₃ is selected from N, NR_{1N}, O, S or CR₅;
A₄ is selected from N, NR_{1N}, O, S or CR₇;
R_{1N} is selected from hydrogen, (1-4C)alkyl, (2-4C)alkanoyl, (3-6C)cycloalkyl, (3-6C)cycloalkyl-(1-2C)alkyl, a carbon-linked 4 to 7 membered heterocyclyl, or 4 to 7 membered heterocyclyl(1-2C)alkyl, and wherein any alkyl, alkanoyl, cycloalkyl, cycloalkyl-alkyl, heterocyclyl, heterocyclyl-alkyl group is optionally substituted with one or more R_{5c} groups defined above;
R₄, R₅ and R₇ are as defined above;
and with the proviso that:
(i) only one of A, A₂, A₃ and A₄ can be O or S;
(ii) only one of A₁, A₂, A₃ and A₄ can be NR_{1N};
(iii) 1 to 3 of A₁, A₂, A₃ and A₄ can be N;
(iii) a group of the formula: 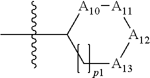
wherein:
p1 is 0 or 1;
A₁₀ is selected from CH₂ or CHR₇;
A₁₁ is selected from CH₂ or CHR₄;
A₁₂ is selected from NR_{5N}, CH₂ or CHR₅;
A₁₃ is selected from CH₂ or CHR₆;
R₄, R₅, R₆ and R₇ are as defined above;
R_{5N} is:
a) hydrogen;
b) a group of the formula: 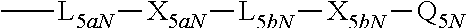
wherein:
L_{5aN} is absent or (1-3C)alkylene;
X_{5aN} is absent or is selected from the group consisting of:
(i) —C(O)—, —S(O)₀₋₂— or —C(O)—N(R₅ₐ₁)— when L_{5aN} is absent; or
(ii) —O—, —C(O)—, —NR₅ₐ₁—, —C(O)—O—, —O—C(O)—, —S(O)O-2—, —C(O)—N(R₅ₐ₁)—, —N(R₅ₐ₁)—C(O)—, —O—C(O)—N(R₅ₐ₁)—, —N(R₅ₐ₁)—C(O)—O— or —N(R₅ₐ₂)—C(O)—NR₅ₐ₁— when L_{5aN} is present;
where R₅ₐ₁ and R₅ₐ₂ are as defined above;
L_{5bN} is absent or (1-2C)alkylene;
X_{5bN} is absent or is selected from the group consisting of —O—, —C(O)—, —NR_{5b1}—, —S(O)₀₋₂—, —C(O)—N(R_{5b1})— or —N(R_{5b1})—C(O)—, where R_{5b}, and R_{5b2} are independently selected from the group consisting of hydrogen or (1-2C)alkyl;
Q_{5N} is selected from the group consisting of:
(i) hydrogen;
(ii) (1-4C)alkyl or (3-6C)cycloalkyl;
and wherein Q_{5N} is optionally substituted with one or more R_{5c} groups as defined above;
and with the proviso that one or two of A₁₀, A₁₁, A₁₂ and A₁₃ can be N;
(iv) a group of the formula: 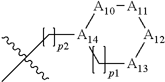
wherein:
p1, A₁₀, A₁₁, A₁₂, and A₁₃ are as defined above;
p2 is 1 to 4;
A₁₄ is C or N;
and with the proviso that one of A₁₀, A₁₁, A₁₂ and A₁₃ can be N.

14. A compound according to any one of the preceding claims, or a pharmaceutically acceptable salt thereof, wherein Q₁ is selected from:
(i) a group of the formula: 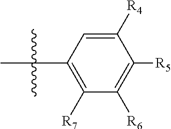
wherein:
R₄ and R₆ are each independently selected from hydrogen, halo, cyano, or a group of the formula: 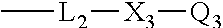
wherein:
L₂ is absent or (1-3C)alkylene;
X₃ is absent or is selected from the group consisting of —O—, —NR₄ₐ—, —C(O)—O—, —O—C(O)—, —S(O)₀₋₂—, —C(O)—N(R₄ₐ)—, —N(R₄ₐ)—C(O)—, —SO₂N(R₄ₐ)— or —N(R₄ₐ)SO₂—, where R₄ₐ and R_{4b} are independently selected from the group consisting of hydrogen or (1-2C)alkyl;
Q₃ is selected from the group consisting of hydrogen, (1-4C)alkyl, (3-6C)cycloalkyl, (3-6C)cycloalkyl(1-2C)alkyl, phenyl, phenyl(1-2C)alkyl, 3 to 8-membered heterocyclyl, [3 to 8-membered heterocyclyl](1-2C)alkyl, 5 or 6-membered heteroaryl and 5 or 6-membered heteroaryl(1-2C)alkyl, wherein any alkyl, cycloalkyl, cycloalkylalkyl, aryl, arylalkyl, heterocyclyl, heterocyclyl-alkyl, heteroaryl or heteroaryl-alkyl in Q₃ is optionally substituted with one or more R₄ₑ groups; and
each R_{4c} group present is independently selected from the group consisting of hydroxy, cyano, oxo, halogen, (1-4C)alkyl, (1-4C)alkoxy, (3-6C)cycloalkyl, (3-6C)cycloalkyl(1-2C)alkyl, —NR_{4d}R₄ₑ, —C(O)—R_{4d}, —C(O)—OR_{4d}, —O—C(O)—R_{4d}, —C(O)—NR_{4d}R₄ₑ, —N(R₄ₑ)C(O)—R_{4d}, —S(O)₀₋₂R_{4d}—, —S(O)₂NR_{4d}R₄ₑ, or —N(R₄ₑ)—S(O)₂R_{4d},
wherein R_{4d} and R₄ₑ are each independently hydrogen or (1-4C)alkyl;
and wherein any alkyl, alkoxy, cycloalkyl, cycloalkyl-alkyl group present in a R_{4c} group, or any alkyl group present in a R_{4d} or R₄ₑ group, is optionally further substituted by one or more substituents independently selected from hydroxy, cyano, halogen, R_{4U}, —OR_{4f}, —NR_{4f}R_{4g} and —C(O)—R_{4f}, wherein R₄r and R_{4g} are both independently selected from hydrogen or (1-2C)alkyl;
R₅ is:
a) hydrogen, halo, cyano; or
b) a group of the formula: 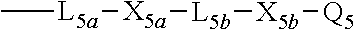
wherein:
L₅ₐ is absent or (1-3C)alkylene;
X₅ₐ is absent or is selected from the group consisting of —O—, —C(O)—, —NR₅ₐ₁—, —C(O)—O—, —O—C(O)—, —S(O)₀₋₂—, —C(O)—N(R₅ₐ₁)—, —N(R₅ₐ₁)—C(O)—, —SO₂N(R₅ₐ₁)— or —N(R₅ₐ₁)SO₂—, where R₅ₐ₁ and R₅ₐ₂ are independently selected from the group consisting of hydrogen or (1-4C)alkyl;
L_{5b} is absent or (1-4C)alkylene;
X_{5b} is absent or is selected from the group consisting of —O—, —C(O)—, —NR_{5b1}—, —C(O)—O—, —O—C(O)—, —S(O)₀₋₂—, —C(O)—N(R_{5b1})—, —N(R_{5b1})—C(O)—, —O—C(O)—N(R_{5b1})—, —N(R_{5b1})—C(O)—O—, —N(R_{5b2})—C(O)—NR_{5b1}—, —SO₂N(R_{5b1})— or —N(R_{5b1})SO₂—, where R_{5b1} and R_{5b2} are independently selected from the group consisting of hydrogen or (1-4C)alkyl;
Q₅ is selected from the group consisting of hydrogen, (1-6C)alkyl, (3-8C)cycloalkyl, (3-8C)cycloalkyl(1-4C)alkyl, phenyl, phenyl(1-2C)alkyl, 3 to 8-membered heterocyclyl, [3 to 8-membered heterocyclyl](1-2C)alkyl, 5 or 6-membered heteroaryl and 5 or 6-membered heteroaryl(1-2C)alkyl and wherein Q₅ is optionally substituted with one or more R_{5c} groups;
and each R_{5c} group present is independently selected from the group consisting of hydroxy, cyano, oxo, halogen, (1-6C)alkyl, (1-6C)alkoxy, (3-8C)cycloalkyl (including spiro-fused (3-8C)cycloalkyl), or (3-8C)cycloalkyl(1-2C)alkyl, —NR_{5d}R₅ₑ, —C(O)—R_{5d}, —C(O)—OR_{5d}, —O—C(O)—R_{5d}, —C(O)—NR_{5d}R₅ₑ, —N(R₅ₑ)C(O)—R_{5d}, —S(O)₀₋₂R_{5d}—, —S(O)₂NR_{5d}R₅ₑ, —N(R₅ₑ)—S(O)₂R_{5d}, phenyl, 5 or 6-membered heteroaryl, or 4 to 8-membered heterocyclyl,
wherein R_{5d} and R₅ₑ are each independently hydrogen, (1-6C)alkyl, (3-6C)cycloalkyl, or (3-6C)cycloalkyl(1-2C)alkyl;
and wherein any alkyl, alkoxy, cycloalkyl, cycloalkyl-alkyl, phenyl, heteroaryl or heterocyclyl group present in a R_{5c} group, or any alkyl, cycloalkyl or cycloalkyl-alkyl group present in a R_{5d} or R₅ₑ group, is optionally further substituted by one or more substituents independently selected from hydroxy, cyano, halo, R_{5f}, —OR_{5f}, —NR_{5f}R_{5g} and —C(O)—R_{5f}, wherein R_{5f} and R_{5g} are both independently selected from hydrogen, (1-4C)alkyl, hydroxy(1-4C)alkyl, halo(1-4C)alkyl, or (1-2C)alkoxy(1-4C)alkyl;
R₁ is selected from hydrogen, hydroxymethyl, or methoxy;
or R₄ and R₅, or R₅ and R₆ are linked to form a fused phenyl, 5- or 6-membered heteroaryl or 5 to 7-membered heterocyclic ring, which is optionally substituted by one or more substituent groups selected from hydroxy, cyano, halo, (1-2C)alkyl, (1-2C)haloalkyl, (1-2C)alkoxy or (1-2C)haloalkoxy;
(ii) a group of the formula: 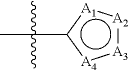
wherein:
A₁ is selected from N, NR_{1N}, O, S or CR₇;
A₂ is selected from N, NR_{1N}, O, S or CR₄;
A₃ is selected from N, NR_{1N}, O, S or CR₅;
A₄ is selected from N, NR_{1N}, O, S or CR₇;
R_{1N} is selected from hydrogen, (1-4C)alkyl, (2-4C)alkanoyl, phenyl, benzyl, (3-8C)cycloalkyl, (3-8C)cycloalkyl-(1-2C)alkyl, a carbon-linked 4 to 7 membered heterocyclyl, or 4 to 7 membered heterocyclyl(1-2C)alkyl, and wherein any alkyl, alkanoyl, cycloalkyl, cycloalkyl-alkyl, phenyl, benzyl, heterocyclyl, heterocyclyl-alkyl group is optionally substituted with one or more R_{5c} groups defined above;
R₄, R₅ and R₇ are as defined above;
and with the proviso that:
(i) only one of A₁, A₂, A₃ and A₄ can be O or S;
(ii) only one of A₁, A₂, A₃ and A₄ can be NR_{1N};
(iii) 1 to 3 of A₁, A₂, A₃ and A₄ can be N;
(iii) a group of the formula: 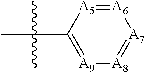
wherein:
A₅ is selected from N or CH;
A₆ is selected from N or CR₄;
A₇ is selected from N or CR₅;
A₉ is selected from N or CR₅;
A₉ is selected from N or CR₇;
R₄, R₅, R₆ and R₇ are as defined above;
and with the proviso that only one of A₅, A₆, A₇, A₈ and A₉ can be N;
(iv) a group of the formula: 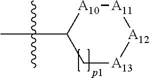
wherein:
p1 is 0 or 1;
A₁₀ is selected from NH, CH₂ or CHR₇;
A₁₁ is selected from NR_{4N}, CH₂ or CHR₄;
A₁₂ is selected from NR_{5N}, CH₂ or CHR₅;
A₁₃ is selected from NR_{6N}, CH₂ or CHR_{O};
R₄, R₅, R₆ and R₇ are as defined above;
R_{4N} and R_{6N} are each independently selected from hydrogen or a group of the formula: 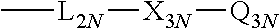
wherein:
L_{2N} is absent or (1-3C)alkylene;
X_{3N} is absent or is selected from the group consisting of:
(i) —C(O)—, —S(O)₀₋₂— or —C(O)—N(R₄ₐ)— when L₂ is absent; or
(ii) —O—, —C(O)—, —NR₄ₐ—, —C(O)—O—, —O—C(O)—, —S(O)₀₋₂—, —C(O)—N(R₄ₐ)—, —N(R₄ₐ)—C(O)—, —O—C(O)—N(R₄ₐ)—, —N(R₄)—C(O)—O—, —N(R_{4b})—C(O)—NR₄ₐ—, —SO₂N(R₄ₐ)— or —N(R₄ₐ)SO₂— when L₂ is (1-3C)alkylene;
wherein R₄ₐ and R_{4b} are as defined above;
Q_{3N} is selected from the group consisting of hydrogen, (1-6C)alkyl, (3-6C)cycloalkyl, (3-6C)cycloalkyl(1-2C)alkyl, heterocyclyl, heterocyclyl(1-2C)alkyl, heteroaryl and heteroaryl(1-2C)alkyl, wherein any alkyl, cycloalkyl, cycloalkylalkyl, heterocyclyl, heterocyclylalkyl, heteroaryl or heteroarylalkyl in Q₃ is optionally substituted with one or more R₄ₑ groups; and
each R₄ₑ group present is as defined above;
R_{5N} is:
a) hydrogen;
b) a group of the formula: 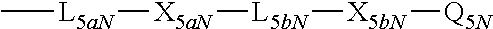
wherein:
L_{5aN} is absent or (1-3C)alkylene;
X_{5aN} is absent or is selected from the group consisting of:
(i) —C(O)—, —S(O)₀₋₂—, —C(O)—N(R₅ₐ₁)— when L_{5aN} is absent; or
(ii) —O—, —C(O)—, —NR₅ₐ₁—, —C(O)—O—, —O—C(O)—, —S(O)O-2—, —C(O)—N(R₅ₐ₁)—, —N(R₅ₐ₁)—C(O)—, —O—C(O)—N(R₅ₐ₁)—, —N(R₅ₐ₁)—C(O)—O—, —N(R₅ₐ₂)—C(O)—NR₅ₐ₁—, —SO₂N(R₅ₐ)— or —N(R₅ₐ₁)SO₂— when L_{5aN} is present;
where R₅ₐ, and R₅ₐ₂ are as defined above:
L_{5bN} is absent or (1-2C)alkylene;
X_{5bN} is absent or is selected from the group consisting of —O—, —C(O)—, —NR_{5b}I—, —C(O)—O—, —O—C(O)—, —S(O)₀₋₂—, —C(O)—N(R_{5b1})—, —N(R_{5b1})—C(O)—, —SO₂N(R_{5b1})— or —N(R_{5b1})SO₂—, where R_{5b}, and R_{5b2} are independently selected from the group consisting of hydrogen or (1-4C)alkyl;
Q_{5N} is selected from the group consisting of:
(i) hydrogen;
(ii) (1-4C)alkyl, (3-6C)cycloalkyl, (3-6C)cycloalkyl(1-1C)alkyl, phenyl, phenyl(1-2C)alkyl, 5- or 6-membered heteroaryl, 5- or 6-membered heteroaryl(1-2C)alkyl;
(iii) a carbon-linked 4 to 8 membered heterocyclyl (when -L_{5aN}, X_{5aN}, L_{5bN}, and X_{5bN} are absent) or a 4 to 8 membered heterocyclyl (when one or more of -L_{5aN}, X_{5aN}, L_{5bN}, and X_{5bN} are present), or [4 to 8 membered heterocyclyl](1-2C)alkyl;
and wherein Q_{5N} is optionally substituted with one or more R₅, groups as defined above;
and with the proviso that one or two of A₁₀, A₁₁, A₁₂ and A₁₃ can be N;
(v) a group of the formula: 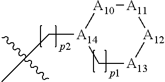
wherein:
p1, A₁₀, A₁₁, A₁₂, and A₁₃ are as defined above;
p2 is 1 to 5;
A₁₄ is C or N;
and with the proviso that one of A₁₀, A₁₁, A₁₂ and A₁₃ can be N.

15. A compound according to any one of the preceding claims, or a pharmaceutically acceptable salt thereof, wherein Q₁ is selected from:
(i) a group of the formula: 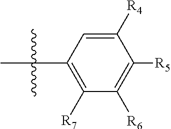
wherein:
R₄ and R₆ are each independently selected from hydrogen, or a group of the formula: 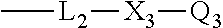
wherein:
L₂ is absent or (1-3C)alkylene;
X₃ is absent or is selected from the group consisting of —O—, —NR₄ₐ—, —S(O)₀₋₂—, —C(O)—N(R₄ₐ)—, —N(R₄ₐ)—C(O)—, —SO₂N(R₄ₐ)— or —N(R₄ₐ)SO₂—, where R₄ₐ and R_{4b} are independently selected from the group consisting of hydrogen or (1-2C)alkyl;
Q₃ is selected from the group consisting of hydrogen, (1-4C)alkyl, (3-6C)cycloalkyl, (3-8C)cycloalkyl(1-4C)alkyl, phenyl, 3 to 8-membered heterocyclyl, 5 or 6-membered heteroaryl or 5 or 6-membered heteroaryl(1-4C)alkyl wherein any alkyl, cycloalkyl, cycloalkylalkyl, phenyl, heterocyclyl, or heteroaryl in Q₃ is optionally substituted with one or more R₄ₑ groups; and
each R_{4c} group present is independently selected from the group consisting of hydroxy, cyano, oxo, halogen, (1-4C)alkyl, (1-4C)alkoxy, (3-6C)cycloalkyl, (3-6C)cycloalkyl(1-2C)alkyl, —NR_{4d}R₄ₑ, —C(O)—R_{4d}, —C(O)—OR_{4d}, —O—C(O)—R_{4d}, —C(O)—NR_{4d}R₄ₑ, —N(R₄ₑ)C(O)—R_{4d}, —S(O)₀₋₂R_{4d}—, —S(O)₂NR_{4d}R₄ₑ, or —N(R₄ₑ)—S(O)₂R_{4d},
wherein R_{4d} and R₄ₑ are each independently hydrogen or (1-4C)alkyl;
R₅ is:
a) hydrogen, halo, cyano; or
b) a group of the formula: 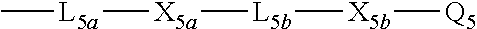
wherein:
L₅ₐ is absent or (1-3C)alkylene;
X₅ₐ is absent or is selected from the group consisting of —O—, —C(O)—, —NR₅ₐ₁—, —S(O)₀₋₂—, —C(O)—N(R₅ₐ₁)—, —N(R₅ₐ₁)—C(O)—, —SO₂N(R₅ₐ₁)— or —N(R₅ₐ₁)SO₂—, where R₅ₐ₁ and R₅ₐ₂ are independently selected from the group consisting of hydrogen or (1-2C)alkyl;
L_{5b} is absent or (1-4C)alkylene;
X_{5b} is absent or is selected from the group consisting of —O—, —C(O)—, —NR_{5b1}—, —S(O)₀₋₂—, —C(O)—N(R_{5b1})—, —N(R_{5b1})—C(O)—, —SO₂N(R_{5b1})— or —N(R_{5b1})SO₂—, where R_{5b1} and R_{5b2} are independently selected from the group consisting of hydrogen or (1-2C)alkyl;
Q₅ is selected from the group consisting of hydrogen, (1-6C)alkyl, (3-8C)cycloalkyl, phenyl, 3 to 8-membered heterocyclyl, or 5 or 6-membered heteroaryl and wherein Q₅ is optionally substituted with one or more R_{5c} groups;
and each R_{5c} group present is independently selected from the group consisting of hydroxy, cyano, halogen, (1-6C)alkyl, (1-6C)alkoxy, (3-8C)cycloalkyl (including spiro-fused (3-8C)cycloalkyl), (3-8C)cycloalkyl(1-2C)alkyl, —NR_{5d}R₅ₑ, —C(O)—R_{5d}, —C(O)—NR_{5d}R₅ₑ, —N(R₅ₑ)C(O)—R_{5d}, —S(O)₀₋₂R_{5d}—, —S(O)₂NR_{5d}R₅ₑ, —N(R₅ₑ)—S(O)₂Rsd, phenyl, 5 or 6-membered heteroaryl, or 4 to 8-membered heterocyclyl,
wherein R_{5d} and R₅ₑ are each independently hydrogen, (1-6C)alkyl, (3-6C)cycloalkyl, or (3-6C)cycloalkyl(1-2C)alkyl;
and wherein any alkyl, alkoxy, cycloalkyl, cycloalkyl-alkyl, phenyl, heteroaryl or heterocyclyl group present in a R_{5c} group, or any alkyl, cycloalkyl or cycloalkyl-alkyl group present in a R_{5d} or R₅ₑ group, is optionally further substituted by one or more substituents independently selected from hydroxy, cyano, halo, R_{5f}, —OR_{5f}, —NR_{5f}R_{5g} and —C(O)—R_{5f}, wherein R_{5f} and R_{5g} are both independently selected from hydrogen, (1-4C)alkyl, hydroxy(1-4C)alkyl, halo(1-4C)alkyl, or (1-2C)alkoxy(1-4C)alkyl;
R₇ is selected from hydrogen, hydroxymethyl, or methoxy;
or R₄ and R₅, or R₅ and R₆ are linked to form a fused phenyl, 5- or 6-membered heteroaryl or 5 to 7-membered heterocyclic ring, which is optionally substituted by one or more substituent groups selected from hydroxy, cyano, halo, (1-2C)alkyl, (1-2C)haloalkyl, (1-2C)alkoxy or (1-2C)haloalkoxy;
(ii) a group of the formula: 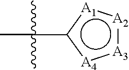
wherein:
A₁ is selected from N, NR_{1N}, O, S or CR₇;
A₂ is selected from N, NR_{1N}, O, S or CR₄;
A₃ is selected from N, NR_{1N}, O, S or CR₅;
A₄ is selected from N, NR_{1N}, O, S or CR₇;
R_{1N} is selected from hydrogen, (1-4C)alkyl, (2-4C)alkanoyl, phenyl, benzyl, (3-8C)cycloalkyl, (3-8C)cycloalkyl-(1-2C)alkyl, a carbon-linked 4 to 7 membered heterocyclyl, or 4 to 7 membered heterocyclyl(1-2C)alkyl, and wherein any alkyl, alkanoyl, cycloalkyl, cycloalkyl-alkyl, phenyl, benzyl, heterocyclyl, heterocyclyl-alkyl group is optionally substituted with one or more R_{5c} groups defined above;
R₄, R₅ and R₇ are as defined above;
and with the proviso that:
(i) only one of A₁, A₂, A₃ and A₄ can be O or S;
(ii) only one of A₁, A₂, A₃ and A₄ can be NR_{1N};
(iii) 1 to 3 of A₁, A₂, A₃ and A₄ can be N;
(iii) a group of the formula: 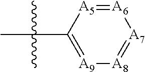
wherein:
A₉ is selected from N or CH;
A₆ is selected from N or CR₄;
A₇ is selected from N or CR₅;
A₉ is selected from N or CR₆;
A₉ is selected from N or CR₇;
R₄, R₅, R₆ and R₇ are as defined above;
and with the proviso that only one of A₅, A₆, A₇, A₈ and A₉ can be N;
(iv) a group of the formula: 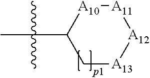
wherein:
p1 is 0 or 1;
A₁₀ is selected from NH, CH₂ or CHR₇;
A₁₁ is selected from NR_{4N}, CH₂ or CHR₄;
A₁₂ is selected from NR_{5N}, CH₂ or CHR₅;
A₁₃ is selected from NR_{6N}, CH₂ or CHR₆;
R₄, R₅, R₆ and R₇ are as defined above;
R_{4N} and R_{6N} are each independently selected from hydrogen or a group of the formula: 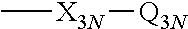
wherein:
X_{3N} is absent or is selected from the group consisting of: —C(O)—, —S(O)₀₋₂— or —C(O)—N(R₄₈)—;
wherein R₄ₐ is as defined above;
Q_{3N} is selected from the group consisting of hydrogen, (1-4C)alkyl, (3-6C)cycloalkyl, (3-6C)cycloalkyl(1-2C)alkyl, heterocyclyl, heteroaryl and, wherein any alkyl, cycloalkyl, cycloalkylalkyl, heterocyclyl or heteroaryl in Q₃ is optionally substituted with one or more R₄ₑ groups; and
each R₄ₑ group present is as defined above;
R_{5N} is:
a) hydrogen;
b) a group of the formula: 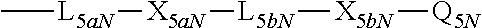
wherein:
L_{5aN} is absent or (1-30)alkylene;
X_{5aN} is absent or is selected from the group consisting of:
(i) —C(O)—, —S(O)₀₋₂—, —C(O)—N(R₅ₐ₁)— when L_{5aN} is absent; or
(ii) —O—, —C(O)—, —NR₅ₐ₁—, —C(O)—O—, —O—C(O)—, —S(O)₀₋₂—, —C(O)—N(R₅ₐ₁)—, —N(R₅ₐ₁)—C(O)—, —O—C(O)—N(R₅ₐ₁)—, —N(R₅ₐ₁)—C(O)—O—, —N(R₅ₐ₂)—C(O)—NR₅ₐ₁—, —SO₂N(R₅ₐ₁)— or —N(R₅ₐ₁)SO₂— when L_{5aN} is present;
where Ra₁ and R₅ₐ₂ are as defined above;
L_{5bN} is absent or (1-2C)alkylene;
X_{5bN} is absent or is selected from the group consisting of —O—, —C(O)—, —NR_{5b1}—, —S(O)₀₋₂—, —C(O)—N(R₅bI)-, —N(R₅bI)—C(O)—, —SO₂N(R_{5b1})— or —N(R_{5b1})SO₂—, where R_{5b1} and R_{5b2} are independently selected from the group consisting of hydrogen or (1-2C)alkyl;
Q_{5N} is selected from the group consisting of:
(i) hydrogen;
(ii) (1-4C)alkyl, (3-6C)cycloalkyl, (3-6C)cycloalkyl(1-1C)alkyl, phenyl, phenyl(1-2C)alkyl, 5- or 6-membered heteroaryl, 5- or 6-membered heteroaryl(1-2C)alkyl;
and wherein Q_{5N} is optionally substituted with one or more R₅, groups as defined above;
and with the proviso that one or two of A₁₀, A₁₁, A₁₂ and A₁₃ can be N;
(v) a group of the formula: 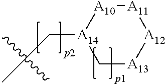
wherein:
p1, A₁₀, A₁₁, A₁₂, and A₁₃ are as defined above;
p2 is 1 to 4;
A₁₄ is C or N;
and with the proviso that one of A₁₀, A₁₁, A₁₂ and A₁₃ can be N.

16. A compound according to any one of the preceding claims, or a pharmaceutically acceptable salt thereof, wherein Q₁ is selected from:
(i) a group of the formula: 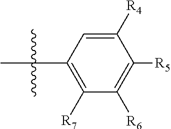
wherein:
R₄ and R₆ are each independently selected from hydrogen, or a group of the formula: 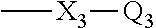
wherein:
X₃ is absent or is selected from the group consisting of —O—, —NR₄ₐ—, —C(O)—N(R₄ₐ)—, —N(R₄ₐ)—C(O)—, —SO₂N(R₄ₐ)— or —N(R₄ₐ)SO₂—, where R₄ₐ and R_{4b} are independently selected from the group consisting of hydrogen or methyl;
Q₃ is selected from the group consisting of hydrogen, (1-4C)alkyl, (3-6C)cycloalkyl, (3-6C)cycloalkyl(1-4C)alkyl, phenyl, 3 to 8-membered heterocyclyl, 5 or 6-membered heteroaryl or 5 or 6-membered heteroaryl(1-4C)alkyl wherein any alkyl, cycloalkyl, cycloalkylalkyl, phenyl, heterocyclyl or heteroaryl in Q₃ is optionally substituted with one or more R₄ₑ groups; and
each R_{4c} group present is independently selected from the group consisting of hydroxy, cyano, oxo, halogen, methyl, methoxy, (3-6C)cycloalkyl, —NR_{4d}R₄ₑ, —C(O)—R_{4d} or —C(O)—OR_{4d},
wherein R_{4d} and R₄ₑ are each independently hydrogen or methyl;
R₅ is:
a) hydrogen, halo, cyano; or
b) a group of the formula: 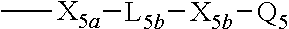
wherein:
X₅ₐ is absent or is selected from the group consisting of —O—, —C(O)—, —NR₅ₐ₁— or —N(R₅ₐ₁)—C(O)—, where R₅ₐ, is independently selected from the group consisting of hydrogen or methyl;
L_{5b} is absent or (1-4C)alkylene;
X_{5b} is absent or is selected from the group consisting of —O—, —C(O)— or —NR_{5b1}—, where R_{5b}, and R_{5b2} are independently selected from the group consisting of hydrogen or (1-2C)alkyl;
Q₅ is selected from the group consisting of hydrogen, (1-6C)alkyl or 3 to 8-membered heterocyclyl and wherein Q₅ is optionally substituted with one or more R_{5c} groups;
and each R₅, group present is independently selected from the group consisting of hydroxy, cyano, halogen, methyl, methoxy
R₇ is selected from hydrogen or methoxy;
(ii) a group of the formula: 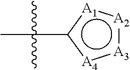
wherein:
A₁ is selected from N, NR_{1N}, O, S or CR₇;
A₂ is selected from N, NR_{1N}, O, S or CR₄;
A₃ is selected from N, NR_{1N}, O, S or CR₅;
A₄ is selected from N, NR_{1N}, O, S or CR₇;
R_{1N} is selected from hydrogen, (1-4C)alkyl, (2-4C)alkanoyl, (3-6C)cycloalkyl, (3-6C)cycloalkyl-(1-2C)alkyl, a carbon-linked 4 to 7 membered heterocyclyl, or 4 to 7 membered heterocyclyl(1-2C)alkyl, and wherein any alkyl, alkanoyl, cycloalkyl, cycloalkyl-alkyl, heterocyclyl, heterocyclyl-alkyl group is optionally substituted with one or more R₅, groups defined above;
R₄, R₅ and R₇ are as defined above;
and with the proviso that:
(i) only one of A₁, A₂, A₃ and A₄ can be O or S;
(ii) only one of A₁, A₂, A₃ and A₄ can be NR_{1N};
(iii) 1 to 3 of A₁, A₂, A₃ and A₄ can be N;
(iii) a group of the formula: 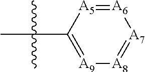
wherein:
A₅ is selected from N or CH;
A₉ is selected from N or CR₄;
A₇ is selected from N or CR₅;
A₉ is selected from N or CR₆;
A₉ is selected from N or CR₇;
R₄, R₅, R₆ and R₇ are as defined above;
and with the proviso that only one of A₅, A₆, A₇, A₈ and A₉ can be N;
(iv) a group of the formula: 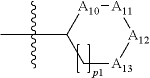
wherein:
p1 is 0 or 1;
A₁₀ is selected from CH₂ or CHR₇;
A₁₁ is selected from CH₂ or CHR₄;
A₁₂ is selected from NR_{5N}, CH₂ or CHR₅;
A₁₃ is selected from CH₂ or CHR₆;
R₄, R₅, R₆ and R₇ are as defined above;
R_{5N} is:
a) hydrogen;
b) a group of the formula: 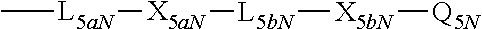
wherein:
L_{5aN} is absent or (1-30)alkylene;
X_{5aN} is absent or is selected from the group consisting of:
(i) —C(O)—, —S(O)₀₋₂— or —C(O)—N(R₅ₐ₁)— when L_{5aN} iS absent; or
(ii) —O—, —C(O)—, —NR₅ₐ₁—, —C(O)—O—, —O—C(O)—, —S(O)O-2—, —C(O)—N(R₅ₐ₁)—, —N(R₅ₐ₁)—C(O)—, —O—C(O)—N(R₅ₐ₁)—, —N(R₅ₐ₁)—C(O)—O— or —N(R₅ₐ₂)—C(O)—NR₅ₐ₁— when L_{5aN} is present;
where R₅ₐ₁ and R₅ₐ₂ are as defined above;
L_{5bN} is absent or (1-2C)alkylene;
X_{5bN} is absent or is selected from the group consisting of —O—, —C(O)—, —NR_{5b1}—, —S(O)₀₋₂—, —C(O)—N(R_{5b1})— or —N(R_{5b1})—C(O)—, where R_{5b1} and R_{5b2} are independently selected from the group consisting of hydrogen or (1-2C)alkyl;
Q_{5N} is selected from the group consisting of:
(i) hydrogen;
(ii) (1-4C)alkyl or (3-6C)cycloalkyl;
and wherein Q_{5N} is optionally substituted with one or more R_{5c} groups as defined above;
and with the proviso that one or two of A₁₀, A₁₁, A₁₂ and A₁₃ can be N;
(v) a group of the formula: 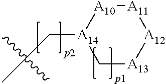
wherein:
p1, A₁₀, A₁₁, A₁₂, and A₁₃ are as defined above;
p2 is 1 to 4;
A₁₄ is C or N;
and with the proviso that one of A₁₀, A₁₁, A₁₂ and A₁₃ can be N.

17. A compound according to any one of the preceding claims, or a pharmaceutically acceptable salt thereof, wherein Q₁ is selected from:
(i) a group of the formula: 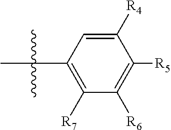
wherein:
R₄ and R₆ are each independently selected from hydrogen, or a group of the formula: 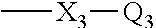
wherein:
X₃ is absent or is selected from the group consisting of —O—, —NR₄ₐ—, —C(O)—N(R₄ₐ)—, —N(R₄ₐ)—C(O)—, —SO₂N(R₄ₐ)— or —N(R₄ₐ)SO₂—, where R₄ₐ and R_{4b} are independently selected from the group consisting of hydrogen or (1-2C)alkyl;
Q₃ is selected from the group consisting of hydrogen, (1-4C)alkyl, (3-6C)cycloalkyl, (3-6C)cycloalkyl(1-4C)alkyl, phenyl, 3 to 8-membered heterocyclyl, 5 or 6-membered heteroaryl or 5 or 6-membered heteroaryl(1-4C)alkyl wherein any alkyl, cycloalkyl, cycloalkylalkyl, phenyl, heterocyclyl or heteroaryl in Q₃ is optionally substituted with one or more R_{4c} groups; and
each R_{4c} group present is independently selected from the group consisting of hydroxy, cyano, oxo, halogen, (1-4C)alkyl, (1-4C)alkoxy, (3-6C)cycloalkyl, (3-6C)cycloalkyl(1-2C)alkyl, —NR_{4d}R₄ₑ, —C(O)—R_{4d}, —C(O)—OR_{4d}, —O—C(O)—R_{4d}, —C(O)—NR_{4d}R₄ₑ, —N(R₄ₑ)C(O)—R_{4d}, —S(O)₀₋₂R_{4d}—, —S(O)₂NR_{4d}R₄ₑ, or —N(R₄ₑ)—S(O)₂R_{4d},
wherein R_{4d} and R₄ₑ are each independently hydrogen or (1-2C)alkyl;
R₅ is:
a) hydrogen, halo, cyano; or
b) a group of the formula: 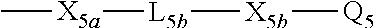
wherein:
X₅ₐ is absent or is selected from the group consisting of —O—, —C(O)—, —NR₅ₐ₁—, —S(O)₀₋₂—, —C(O)—N(R₅ₐ₁)—, —N(R₅ₐ₁)—C(O)—, —SO₂N(R₅ₐ₁)— or —N(R₅ₐ₁)SO₂—, where R₅ₐ₁ and R₅ₐ₂ are independently selected from the group consisting of hydrogen or (1-2C)alkyl;
L_{5b} is absent or (1-4C)alkylene;
X_{5b} is absent or is selected from the group consisting of —O—, —C(O)—, —NR_{5b1}—, —S(O)₀₋₂—, —C(O)—N(R_{5b1})—, —N(R_{5b1})—C(O)—, —SO₂N(R_{5b1})— or —N(R_{5b1})SO₂—, where R_{5b1} and R_{5b2} are independently selected from the group consisting of hydrogen or (1-2C)alkyl;
Q₅ is selected from the group consisting of hydrogen, (1-6C)alkyl, (3-8C)cycloalkyl, phenyl, 3 to 8-membered heterocyclyl, or 5 or 6-membered heteroaryl and wherein Q₅ is optionally substituted with one or more R_{5c} groups;
and each R₅, group present is independently selected from the group consisting of hydroxy, cyano, halogen, (1-6C)alkyl, (1-6C)alkoxy, (3-8C)cycloalkyl (including spiro-fused (3-8C)cycloalkyl), (3-8C)cycloalkyl(1-2C)alkyl, —NR_{5d}R₅ₑ, —C(O)—R_{5d}, —C(O)—NR_{5d}R₅ₑ, —N(R₅ₑ)C(O)—R_{5d}, —S(O)₀₋₂R_{5d}—, —S(O)₂NR_{5d}R₅ₑ, —N(R₅ₑ)—S(O)₂R_{5d}, phenyl, 5 or 6-membered heteroaryl, or 4 to 8-membered heterocyclyl,
wherein R_{5d} and R₅ₑ are each independently hydrogen, (1-6C)alkyl, (3-6C)cycloalkyl, or (3-6C)cycloalkyl(1-2C)alkyl;
and wherein any alkyl, alkoxy, cycloalkyl, cycloalkyl-alkyl, phenyl, heteroaryl or heterocyclyl group present in a R_{5c} group, or any alkyl, cycloalkyl or cycloalkyl-alkyl group present in a R_{5d} or R₅ₑ group, is optionally further substituted by one or more substituents independently selected from hydroxy, cyano, halo, R_{5f}, —OR_{5f}, —NR_{5f}R_{5g} and —C(O)—R_{5f}, wherein R_{5f} and R_{5g} are both independently selected from hydrogen, (1-4C)alkyl, hydroxy(1-4C)alkyl, halo(1-4C)alkyl, or (1-2C)alkoxy(1-4C)alkyl;
R₇ is selected from hydrogen, hydroxymethyl, or methoxy;
or R₄ and R₅, or R₅ and R₆ are linked to form a fused phenyl, 5- or 6-membered heteroaryl or 5 to 7-membered heterocyclic ring, which is optionally substituted by one or more substituent groups selected from hydroxy, cyano, halo, (1-2C)alkyl, (1-2C)haloalkyl, (1-2C)alkoxy or (1-2C)haloalkoxy;
(ii) a group of the formula: 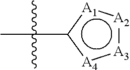
wherein:
A₁ is selected from N, NR_{1N}, O, S or CR₇;
A₂ is selected from N, NR_{1N}, O, S or CR₄;
A₃ is selected from N, NR_{1N}, O, S or CR₅;
A₄ is selected from N, NR_{1N}, O, S or CR₇;
R_{1N} is selected from hydrogen, (1-4C)alkyl, (2-4C)alkanoyl, (3-6C)cycloalkyl, (3-6C)cycloalkyl-(1-2C)alkyl, a carbon-linked 4 to 7 membered heterocyclyl, or 4 to 7 membered heterocyclyl(1-2C)alkyl, and wherein any alkyl, alkanoyl, cycloalkyl, cycloalkyl-alkyl, heterocyclyl, heterocyclyl-alkyl group is optionally substituted with one or more R_{5c} groups defined above;
R₄, R₅ and R₇ are as defined above;
and with the proviso that:
(i) only one of A₁, A₂, A₃ and A₄ can be O or S;
(ii) only one of A₁, A₂, A₃ and A₄ can be NR_{1N};
(iii) 1 to 3 of A₁, A₂, A₃ and A₄ can be N;
(iii) a group of the formula: 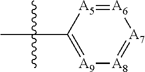
wherein:
A₅ is selected from N or CH;
A₉ is selected from N or CR₄;
A₇ is selected from N or CR₅;
A₈ is selected from N or CR₅;
A₉ is selected from N or CR₇;
R₄, R₅, R₆ and R₇ are as defined above;
and with the proviso that only one of A₅, A₆, A₇, A₈ and A₉ can be N;
(iv) a group of the formula: 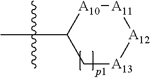
wherein:
p1 is 0 or 1;
A₁₀ is selected from CH₂ or CHR₇;
A₁₁ is selected from CH₂ or CHR₄;
A₁₂ is selected from NR_{5N}, CH₂ or CHRs;
A₁₃ is selected from CH₂ or CHRs;
R₄, R₅, R₆ and R₇ are as defined above;
R_{5N} is:
a) hydrogen;
b) a group of the formula: 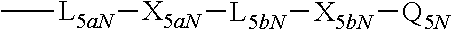
wherein:
L_{5aN} is absent or (1-3C)alkylene;
X_{5aN} is absent or is selected from the group consisting of:
(i) —C(O)—, —S(O)₀₋₂—, —C(O)—N(R₅ₐ₁)— when L_{5aN} is absent; or
(ii) —O—, —C(O)—, —NR₅ₐ₁—, —C(O)—O—, —O—C(O)—, —S(O)₀₋₂—, —C(O)—N(R₅ₐ₁)—, —N(R₅ₐ₁)—C(O)—, —O—C(O)—N(R₅ₐ₁)—, —N(R₅ₐ₁)—C(O)—O—, —N(R₅ₐ₂)—C(O)—NR₅ₐ₁—, —SO₂N(R₅ₐ₁)— or —N(R₅ₐ₁)SO₂— when L_{5aN} is present;
where R₅ₐ₁ and R₅ₐ₂ are as defined above;
L_{5bN} is absent or (1-20)alkylene;
X_{5bN} is absent or is selected from the group consisting of —O—, —C(O)—, —NR_{5b1}—, —S(O)₀₋₂—, —C(O)—N(R_{5b1})—, —N(R_{5b1})—C(O)—, —SO₂N(R_{5b1})— or —N(R_{5b1})SO₂—, where R_{5b1} and R_{5b2} are independently selected from the group consisting of hydrogen or (1-2C)alkyl;
Q_{5N} is selected from the group consisting of:
(i) hydrogen;
(ii) (1-4C)alkyl, (3-6C)cycloalkyl, (3-6C)cycloalkyl(1-1C)alkyl, phenyl, phenyl(1-2C)alkyl, 5- or 6-membered heteroaryl, 5- or 6-membered heteroaryl(1-2C)alkyl;
and wherein Q_{5N} is optionally substituted with one or more R_{5c} groups as defined above;
and with the proviso that one or two of A₁₀, A₁₁, A₁₂ and A₁₃ can be N;
(v) a group of the formula: 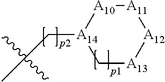
wherein:
p1, A₁₀, A₁₁, A₁₂, and A₁₃ are as defined above;
p2 is 1 to 4;
A₁₄ is C or N;
and with the proviso that one of A₁₀, A₁₁, A₁₂ and A₁₃ can be N.

18. A compound according to any one of the preceding claims, or a pharmaceutically acceptable salt thereof, wherein Q₁ is selected from:
(i) a group of the formula: 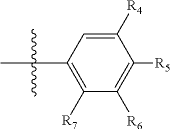
wherein:
R₄ and R₆ are each independently selected from hydrogen, or a group of the formula: 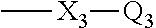
wherein:
X₃ is absent or is selected from the group consisting of —O—, —NR₄ₐ—, —C(O)—N(R₄ₐ)—, —N(R₄ₐ)—C(O)—, —SO₂N(R₄ₐ)— or —N(R₄ₐ)SO₂—, where R₄ₐ and R_{4b} are independently selected from the group consisting of hydrogen or methyl;
Q₃ is selected from the group consisting of hydrogen, (1-4C)alkyl, (3-6C)cycloalkyl, (3-6C)cycloalkyl(1-4C)alkyl, phenyl, 3 to 8-membered heterocyclyl, 5 or 6-membered heteroaryl or 5 or 6-membered heteroaryl(1-4C)alkyl wherein any alkyl, cycloalkyl, cycloalkylalkyl, phenyl, heterocyclyl or heteroaryl in Q₃ is optionally substituted with one or more R_{4c} groups; and
each R_{4c} group present is independently selected from the group consisting of hydroxy, cyano, oxo, halogen, methyl, methoxy, (3-6C)cycloalkyl, —NR_{4d}R₄ₑ, —C(O)—R_{4d} or —C(O)—OR_{4d},
wherein R_{4d} and R₄ₑ are each independently hydrogen or methyl;
R₅ is:
a) hydrogen, halo, cyano; or
b) a group of the formula: 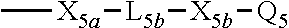
wherein:
X₅ₐ is absent or is selected from the group consisting of —O—, —C(O)—, —NR₅ₐ₁— or —N(R₅ₐ₁)—C(O)—, where R₅ₐ₁ is independently selected from the group consisting of hydrogen or methyl;
L_{5b} is absent or (1-4C)alkylene;
X_{5b} is absent or is selected from the group consisting of —O—, —C(O)— or —NR_{5b1}—, where R_{5b}, and R_{5b2} are independently selected from the group consisting of hydrogen or (1-2C)alkyl;
Q₅ is selected from the group consisting of hydrogen, (1-6C)alkyl or 3 to 8-membered heterocyclyl and wherein Q₅ is optionally substituted with one or more R_{5c} groups;
and each R₅. group present is independently selected from the group consisting of hydroxy, cyano, halogen, methyl, methoxy
R₇ is selected from hydrogen or methoxy;
(ii) a group of the formula: 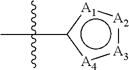
wherein:
A₁ is selected from N, NR_{1N}, O, S or CR₇;
A₂ is selected from N, NR_{1N}, O, S or CR₄;
A₃ is selected from N, NR_{1N}, O, S or CR₅;
A₄ is selected from N, NR_{1N}, O, S or CR₇;
R_{1N} is selected from hydrogen, (1-4C)alkyl, (2-4C)alkanoyl, (3-6C)cycloalkyl, (3-6C)cycloalkyl-(1-2C)alkyl, a carbon-linked 4 to 7 membered heterocyclyl, or 4 to 7 membered heterocyclyl(1-2C)alkyl, and wherein any alkyl, alkanoyl, cycloalkyl, cycloalkyl-alkyl, heterocyclyl, heterocyclyl-alkyl group is optionally substituted with one or more R_{5c} groups defined above;
R₄, R₅ and R₇ are as defined above;
and with the proviso that:
(i) only one of A₁, A₂, A₃ and A₄ can be O or S;
(ii) only one of A₁, A₂, A₃ and A₄ can be NR_{1N};
(iii) 1 to 3 of A₁, A₂, A₃ and A₄ can be N;
(iii) a group of the formula: 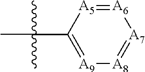
wherein:
A₅ is selected from N or CH;
A₉ is selected from N or CR₄;
A₇ is selected from N or CR₅;
A₉ is selected from N or CR₆;
A₉ is selected from N or CR₇;
R₄, R₅, R₆ and R₇ are as defined above;
and with the proviso that only one of A₅, A₆, A₇, A₈ and A₉ can be N;
(iv) a group of the formula: 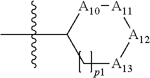
wherein:
p1 is 0 or 1;
A₁₀ is selected from CH₂ or CHR₇;
A₁₁ is selected from CH₂ or CHR₄;
A₁₂ is selected from NR_{5N}, CH₂ or CHR₅;
A₁₃ is selected from CH₂ or CHRs;
R₄, R₅, R₆ and R₇ are as defined above;
R_{5N} is:
a) hydrogen;
b) a group of the formula: 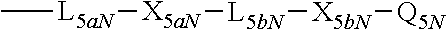
wherein:
L_{5aN} is absent or (1-30)alkylene;
X_{5aN} is absent or is selected from the group consisting of:
(iii) —C(O)—, —S(O)₀₋₂— or —C(O)—N(R₅ₐ₁)— when L_{5aN} is absent; or
(iv) —O—, —C(O)—, —NR₅ₐ₁—, —C(O)—O—, —O—C(O)—, —S(O)₀₋₂—, —C(O)—N(R₅ₐ₁)—, —N(R₅ₐ₁)—C(O)—, —O—C(O)—N(R₅ₐ₁)—, —N(R₅ₐ₁)—C(O)—O— or —N(R₅ₐ₂)—C(O)—NR₅ₐ₁— when L_{5aN} is present;
where Ra₁ and R₅ₐ₂ are as defined above;
L_{5bN} is absent or (1-2C)alkylene;
X_{5bN} is absent or is selected from the group consisting of —O—, —C(O)—, —NR_{5b1}—, —S(O)₀₋₂—, —C(O)—N(R_{5b1})— or —N(R_{5b1})—C(O)—, where R_{5b1} and R_{5b2} are independently selected from the group consisting of hydrogen or (1-2C)alkyl;
Q_{5N} is selected from the group consisting of:
(iii) hydrogen;
(iv) (1-4C)alkyl or (3-6C)cycloalkyl;
and wherein Q_{5N} is optionally substituted with one or more R_{5c} groups as defined above;
and with the proviso that one or two of A₁₀, A₁₁, A₁₂ and A₁₃ can be N;
(v) a group of the formula: 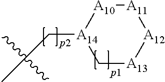
wherein:
p1, A₁₀, A₁₁, A₁₂, and A₁₃ are as defined above;
p2 is 1 to 4;
A₁₄ is C or N;
and with the proviso that one of A₁₀, A₁₁, A₁₂ and A₁₃ can be N.

19. A compound according to any one of the preceding claims, or a pharmaceutically acceptable salt thereof, wherein Q₁ is a group of the formula:
wherein: 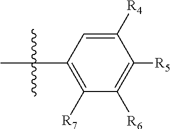
R₄ and R₆ are each independently selected from hydrogen, or a group of the formula: 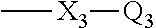
wherein:
X₃ is absent or is selected from the group consisting of —O—, —NR₄ₐ—, —C(O)—N(R₄ₐ)—, —N(R₄ₐ)—C(O)—, —SO₂N(R₄ₐ)— or —N(R₄ₐ)SO₂—, where R₄ₐ and R_{4b} are independently selected from the group consisting of hydrogen or methyl;
Q₃ is selected from the group consisting of hydrogen, (1-4C)alkyl, (3-6C)cycloalkyl, (3-6C)cycloalkyl(1-4C)alkyl, phenyl, 3 to 8-membered heterocyclyl, 5 or 6-membered heteroaryl or 5 or 6-membered heteroaryl(1-4C)alkyl wherein any alkyl, cycloalkyl, cycloalkylalkyl, phenyl, heterocyclyl or heteroaryl in Q₃ is optionally substituted with one or more R_{4c} groups; and
each R_{4c} group present is independently selected from the group consisting of hydroxy, cyano, oxo, halogen, methyl, methoxy, (3-6C)cycloalkyl, —NR_{4d}R₄ₑ, —C(O)—R_{4d} or —C(O)—OR_{4d},
wherein R_{4d} and R₄ₑ are each independently hydrogen or methyl;
R₅ is hydrogen or a group of the formula: 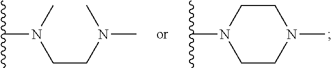
and
R₁ is selected from hydrogen or methoxy.

20. A compound according to any one of the preceding claims, or a pharmaceutically acceptable salt thereof, wherein the compound has one of the formulae Ia or Ib shown below: 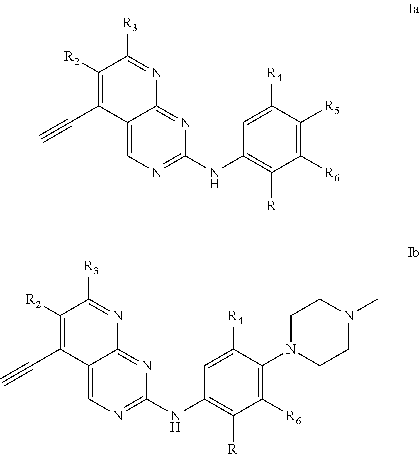
wherein R₂, R₃, R₄, R₅, R₆ and R₇ are as defined in any one of the preceding claims.

21. A compound according to any one of the preceding claims, or a pharmaceutically acceptable salt thereof, wherein the compound is selected from any one of the following:
5-ethynyl-N-(2-methoxyphenyl)pyrido[2,3-d]pyrimidin-2-amine;
N¹-(2-(dimethylamino)ethyl)-N⁴-(5-ethynyl-7-methoxypyrido[2,3-d]pyrimidin-2-yl)-N¹-methylbenzene-1,4-diamine;
5-ethynyl-7-methoxy-N-(4-(4-methylpiperazin-1-yl)phenyl)pyrido[2,3-d]pyrimidin-2-amine;
N-(3-{[cyclobutyl(methyl)amino]methyl}phenyl)-5-ethynyl-7-methoxypyrido[2,3-d]pyrimidin-2-amine;
N³-{5-ethynyl-7-methoxypyrido[2,3-d]pyrimidin-2-yl}-N¹, N-dimethyl-6-(4-methylpiperazin-1-yl)benzene-1.3-diamine;
N¹-{5-ethynyl-7-methoxypyrido[2,3-d]pyrimidin-2-yl}-4-(4-methylpiperazin -1-yl)-N³-phenylbenzene-1,3-diamine;
N-{[3-({5-ethynyl-7-methoxypyrido[2,3-d]pyrimidin-2-yl}amino)phenyl]methyl}-N-methylacetamide;
N³-cyclobutyl-N¹-{5-ethynyl-7-methoxypyrido[2,3-d]pyrimidin-2-yl}-4-(4-methylpiperazin-1-yl)benzene-1,3-diamine;
N-{3-[(dimethylamino)methyl]phenyl}-5-ethynyl-7-methoxypyrido[2,3-d]pyrimidin-2-amine;
N³-(cyclobutylmethyl)-N¹-{5-ethynyl-7-methoxypyrido[2,3-d]pyrimidin-2-yl}-4-(4-methylpiperazin-1-yl)benzene-1,3-diamine;
N¹-{5-ethynyl-7-methoxypyrido[2,3-d]pyrimidin-2-yl}-4-(4-methylpiperazin-1-yl)-N³-[(1-methylpyrazol-3-yl)methyl]benzene-1,3-diamine;
5-ethynyl-N-[4-(4-methylpiperazin-1-yl)phenyl]-7-phenoxypyrido[2,3-d]pyrimidin-2-amine;
5-ethynyl-N-[4-(4-methylpiperazin-1-yl)phenyl]-7-(1,3-thiazol-4-ylmethoxy)pyrido[2,3-d]pyrimidin-2-amine;
7-(cyclohexyloxy)-5-ethynyl-N-(4-(4-methylpiperazin-1-yl)phenyl)pyrido[2,3-d]pyrimidin-2-amine;
5-ethynyl-N-(4-(4-methylpiperazin-1-yl) phenyl)-7-((tetrahydro-2H-pyran-4-yl)oxy) pyrido[2, 3-d]pyrimidin-2-amine;
7-cyclopropoxy-5-ethynyl-N-[4-(4-methylpiperazin-1-yl) phenyl]pyrido[2,3-d]pyrimidin-2-amine;
5-ethynyl-7-isopropoxy-N-[4-(4-methylpiperazin-1-yl)phenyl]pyrido[2,3-d]pyrimidin-2-amine;
7-cyclobutoxy-5-ethynyl-N-[4-(4-methylpiperazin-1-yl)phenyl]pyrido[2,3-d]pyrimidin-2-amine;
7-cyclopropoxy-5-ethynyl-N-[4-(4-methylpiperazin-1-yl) phenyl]pyrido[2,3-d]pyrimidin-2-amine;
7-(benzyloxy)-5-ethynyl-N-[4-(4-methylpiperazin-1-yl)phenyl]pyrido[2,3-d]pyrimidin-2-amine;
5-ethynyl-N-[4-(4-methylpiperazin-1-yl)phenyl]-7-(2-phenylethoxy)pyrido[2,3-d]pyrimidin-2-amine;
5-ethynyl-N-[4-(4-methylpiperazin-1-yl)phenyl]-7-(3-phenylpropoxy)pyrido[2,3-d]pyrimidin-2-amine
7-(cyclopentylmethoxy)-5-ethynyl-6-methyl-N-[4-(4-methylpiperazin-1-yl) phenyl]pyrido[2,3-d]pyrimidin-2-amine;
N²-(4-{[2-(dimethylamino)ethyl](methyl)amino}phenyl)-5-ethynyl-N⁷,N⁷-dimethylpyrido[2,3-d]pyrimidine-2,7-diamine;
N²-(4-{[2-(dimethylamino)ethyl](methyl)amino}phenyl)-5-ethynyl-N⁷-phenyl)pyrido[2,3-d]pyrimidine-2,7-diamine;
5-ethynyl-N⁷-(1-methylmidazol-2-yl)-N²-[4-(4-methylpiperazin-1-yl)phenyl]pyrido[2,3-d]pyrimidine-2,7-diamine;
N⁷-(1-cyclopentylmidazol-2-yl)-5-ethynyl-N²-[4-(4-methylpiperazin-1-yl)phenyl]pyrido[2,3-d]pyrimidine-2,7-diamine;
N-{2-[(4-{[2-(dimethylamino)ethyl](methyl)amino}phenyl)amino]-5-ethynyl)pyrido[2,3-d]pyrimidin-7-yl}acetamide;
N-(5-ethynyl-2-{[4-(4-methylpiperazin-1-yl)phenyl]amino}pyrido[2,3-d]pyrimidin-7-yl)benzamide;
N-(5-ethynyl-2-{[4-(4-methylpiperazin-1-yl)phenyl]amino}pyrido[2,3-d]pyrimidin-7-yl)-2-phenylacetamide;
1-(5-ethynyl-2-{[4-(4-methylpiperazin-1-yl)phenyl]amino}pyrido[2,3-d]pyrimidin-7-yl)pyrrolidin-2-one;
N-(5-ethynyl-2-{[4-(4-methylpiperazin-1-yl)phenyl]amino}pyrido[2,3-d]pyrimidin-7-yl)-3-phenylpropanamide;
N-{2-[(4-{[2-(dimethylamino)ethyl](methyl)amino}phenyl)amino]-5-ethynyl)pyrido[2,3-d]pyrimidin-7-yl}methanesulfonamide;
N¹-[2-(dimethylamino)ethyl]-N⁴-[5-ethynyl-7-(4-methylpyrazol-1-yl)pyrido[2,3-d]pyrimidin-2-yl]-N¹-methylbenzene-1,4-diamine;
5-ethynyl-7-(imidazol-1-yl)-N-[4-(4-methylpiperazin-1-yl)phenyl]pyrido[2,3-d]pyrimidin-2-amine;
5-ethynyl-N-[4-(4-methylpiperazin-1-yl)phenyl]-7-(pyrrol-1-yl)pyrido[2,3-d]pyrimidin-2-amine;
1-(5-ethynyl-2-{[4-(4-methylpiperazin-1-yl)phenyl]amino}pyrido[2,3-d]pyrimidin-7-yl)-3,3-dimethylurea;
1-cyclopentyl-3-(5-ethynyl-2-((4-(4-methylpiperazin-1-yl)phenyl)amino)pyrido[2,3-d]pyrimidin-7-yl)urea;
1-benzyl-3-(5-ethynyl-2-{[4-(4-methylpiperazin-1-yl)phenyl]amino}pyrido[2,3-d]pyrimidin-7-yl)urea;
3-(cyclopentylmethyl)-1-(5-ethynyl-2-{[4-(4-methylpiperazin-1-yl)phenyl]amino}pyrido[2,3-d]pyrimidin-7-yl)urea;
3-cyclobutyl-1-(5-ethynyl-2-{[4-(4-methylpiperazin-1-yl)phenyl]amino}pyrido[2,3-d]pyrimidin-7-yl)urea;
1-(5-ethynyl-2-{[4-(4-methylpiperazin-1-yl)phenyl]amino}pyrido[2,3-d]pyrimidin-7-yl)-3-(oxolan-3-yl)urea;
1-(5-ethynyl-2-{[4-(4-methylpiperazin-1-yl)phenyl]amino}pyrido[2,3-d]pyrimidin-7-yl)-3-(oxetan-3-yl)urea;
3-cyclopropyl-1-(5-ethynyl-2-{[4-(4-methylpiperazin-1-yl)phenyl]amino}pyrido[2,3-d]pyrimidin-7-yl)urea;
1-(5-ethynyl-2-{[4-(4-methylpiperazin-1-yl)phenyl]amino}pyrido[2,3-d]pyrimidin-7-yl)-3-(1,3-oxazol-5-ylmethyl)urea;
1-(5-ethynyl-2-{[4-(4-methylpiperazin-1-yl)phenyl]amino}pyrido[2,3-d]pyrimidin-7-yl)-3-[(3-methylmidazol-4-yl)methyl]urea;
1-(5-ethynyl-2-{[4-(4-methylpiperazin-1-yl)phenyl]amino}pyrido[2,3-d]pyrimidin-7-yl)-3-(1,2-oxazol-5-ylmethyl)urea;
1-(5-ethynyl-2-{[4-(4-methylpiperazin-1-yl)phenyl]amino}pyrido[2,3-d]pyrimidin-7-yl)-3-[(2-methylpyrazol-3-yl)methyl]urea;
3-(5-ethynyl-2-{[4-(4-methylpiperazin-1-yl)phenyl]amino}pyrido[2,3-d]pyrimidin-7-yl)-1-(pyridin-2-ylmethyl)urea;
1-(5-ethynyl-2-{[4-(4-methylpiperazin-1-yl)phenyl]amino}pyrido[2,3-d]pyrimidin-7-yl)-3-(oxolan-2-ylmethyl)urea;
1-(5-ethynyl-2-{[4-(4-methylpiperazin-1-yl)phenyl]amino}pyrido[2,3-d]pyrimidin-7-yl)-3-(oxolan-3-ylmethyl)urea;
3-[(3R)-1-acetylpyrrolidin-3-yl]-1-(5-ethynyl-2-{[4-(4-methylpiperazin-1-yl)phenyl]amino}pyrido[2,3-d]pyrimidin-7-yl)urea;
3-[(3S)-1-acetylpyrrolidin-3-yl]-1-(5-ethynyl-2-{[4-(4-methylpiperazin-1-yl)phenyl]amino}pyrido[2,3-d]pyrimidin-7-yl)urea;
3-[(3R)-1-acetylpyrrolidin-3-yl]-1-(5-ethynyl-2-{[4-(4-methylpiperazin-1-yl)phenyl]amino}pyrido[2,3-d]pyrimidin-7-yl)urea;
1-(5-ethynyl-2-{[4-(4-methylpiperazin-1-yl)phenyl]amino}pyrido[2,3-d]pyrimidin-7-yl)-3-(oxetan-3-ylmethyl)urea;
N-(5-ethynyl-2-{[4-(4-methylpiperazin-1-yl)phenyl]amino}pyrido[2,3-d]pyrimidin-7-yl)pyrrolidine-1-carboxamide;
1-(5-ethynyl-2-{[4-(4-methylpiperazin-1-yl)phenyl]amino}pyrido[2,3-d]pyrimidin-7-yl)-3-(1-methanesulfonylazetidin-3-yl)urea;
N-(5-ethynyl-2-{[4-(4-methylpiperazin-1-yl)phenyl]amino}pyrido[2,3-d]pyrimidin-7-yl)-1,3-dihydroisoindole-2-carboxamide;
1-(5-ethynyl-2-{[4-(4-methylpiperazin-1-yl)phenyl]amino}pyrido[2,3-d]pyrimidin-7-yl)-3-(1H-imidazol-2-yl)urea;
1-(5-ethynyl-2-((4-(4-methylpiperazin-1-yl)phenyl)amino)pyrido[2,3-d]pyrimidin-7-yl)-3-(1-(trifluoromethyl)cyclopentyl)urea;
1-(5-ethynyl-2-((4-(4-methylpiperazin-1-yl)phenyl)amino)pyrido[2,3-d]pyrimidin-7-yl)-3-(3-methyloxetan-3-yl)urea;
1-(5-ethynyl-2-((4-(4-methylpiperazin-1-yl)phenyl)amino)pyrido[2,3-d]pyrimidin-7-yl)-3-(3-methyloxetan-3-yl)urea;
1-(2-((3-((cyclobutylmethyl)amino)-4-(4-methylpiperazin-1-yl)phenyl)amino)-5-ethynyl)pyrido[2,3-d]pyrimidin-7-yl)-3-cyclopentylurea;
1-(5-ethynyl-2-{[4-(4-methylpiperazin-1-yl)phenyl]amino}pyrido[2,3-d]pyrimidin-7-yl)-5-isopropylmidazolidin-2-one;
cyclopentyl (5-ethynyl-2-((4-(4-methylpiperazin-1-yl)phenyl)amino)pyrido[2,3-d]pyrimidin-7-yl)carbamate;
cyclohexyl (5-ethynyl-2-((4-(4-methylpiperazin-1-yl)phenyl)amino)pyrido[2,3-d]pyrimidin-7-yl)carbamate;
benzyl (5-ethynyl-2-((4-(4-methylpiperazin-1-yl)phenyl)amino)pyrido[2,3-d]pyrimidin-7-yl)carbamate;
3-(5-ethynyl-2-{[4-(4-methylpiperazin-1-yl)phenyl]amino}pyrido[2,3-d]pyrimidin-7-yl)-4-isopropyl-1,3-oxazolidin-2-one;
(4R)-3-(5-ethynyl-2-{[4-(4-methylpiperazin-1-yl)phenyl]amino}pyrido[2,3-d]pyrimidin-7-yl)-4-isopropyl-1,3-oxazolidin-2-one;
(4S)-3-(5-ethynyl-2-{[4-(4-methylpiperazin-1-yl) phenyl]amino}pyrido[2, 3-d]pyrimidin-7-yl)-4-isopropyl-1,3-oxazolidin-2-one;
3-(5-ethynyl-2-{[4-(4-methylpiperazin-1-yl)phenyl]amino}pyrido[2,3-d]pyrimidin-7-yl)-1,3-oxazolidin-2-one;
3-Cyclopentyl-1-(5-ethynyl-6-methyl-2-{[4-(4-methylpiperazin-1-yl)phenyl]amino}pyrido[2,3-d]pyrimidin-7-yl)urea;
3-Cyclopentyl-1-(5-ethynyl-2-{[4-(4-methylpiperazin-1-yl)phenyl]amino}pyrido[2, 3-d]pyrimidin-7-yl)-1-methylurea;
3-Cyclopentyl-1-(5-ethynyl-2-{[4-(4-methylpiperazin-1-yl)phenyl]amino}pyrido[2, 3-d]pyrimidin-7-yl)-3-methylurea;
3-Cyclopentyl-1-(5-ethynyl-2-([4-(morpholin-4-yl)phenyl]aminopyrido[2,3-d]pyrimidin-7-yl)urea;
3-Cyclopentyl-1-[5-ethynyl-2-({4-[4-(oxetan-3-yl)piperazin-1-yl]phenyl}amino)pyrido[2,3-d]pyrimidin-7-yl]urea;
N-[4-((7-[(Cyclopentylcarbamoyl)amino]-5-ethynyl)pyrido[2,3-d]pyrimidin-2-ylamino)phenyl]-N-methylacetamide;
2-(Azetidin-1-yl)-N-[4-((7-[(cyclopentylcarbamoyl)amino]-5-ethynyl)pyrido[2,3-d]pyrimidin-2-ylamino)phenyl]-N-methylacetamide;
3-Cyclopentyl-1-[2-((4-[2-(dimethylamino)ethoxy]phenylamino)-5-ethynyl)pyrido[2,3-d]pyrimidin-7-yl]urea;
3-Cyclopentyl-1-[5-ethynyl-2-((4-[(1-methylazetidin-3-yl)oxy]phenylamino)pyrido[2,3-d]pyrimidin-7-yl]urea;
3-Cyclopentyl-1-{5-ethynyl-2-[(4-methanesulfonylphenyl)amino]pyrido[2,3-d]pyrimidin-7-yl}urea;
3-Cyclopentyl-1-(2-([4-(dimethylsulfamoyl)phenyl]amino-5-ethynyl)pyrido[2,3-d]pyrimidin-7-yl)urea;
N-[4-({7-[(Cyclopentylcarbamoyl)amino]-5-ethynyl)pyrido[2,3-d]pyrimidin-2-yl}amino)phenyl]-2-(dimethylamino)-N-methylacetamide;
3-Cyclopentyl-1-(2-[(4-([2-(dimethylamino)ethyl](methyl)sulfamoyl)phenyl)amino]-5-ethynyl)pyrido[2,3-d]pyrimidin-7-ylurea;
3-Cyclopentyl-1-(5-ethynyl-2-[(3-([3-(methanesulfonylmethyl)azetidin-1-yl]methyl)phenyl)amino]pyrido[2,3-d]pyrimidin-7-ylurea;
3-Cyclopentyl-1-(5-ethynyl-2-[(3-([3-(methanesulfonylmethyl)pyrrolidin-1-yl]methyl)phenyl)amino]pyrido[2,3-d]pyrimidin-7-ylurea;
3-Cyclopentyl-1-(5-ethynyl-2-[(3-([4-(methanesulfonylmethyl)piperidin-1-yl]methyl)phenyl)amino]pyrido[2,3-d]pyrimidin-7-ylurea;
3-Cyclopentyl-1-{5-ethynyl-2-[(2-methyl-3,4-dihydro-1H-isoquinolin-5-yl)amino]pyrido[2,3-d]pyrimidin-7-yl}urea;
3-Cyclopentyl-1-[5-ethynyl-2-((2-[3-(methanesulfonylmethyl)pyrrolidin-1-yl]phenylamino)pyrido[2,3-d]pyrimidin-7-yl]urea;
3-Cyclopentyl-1-[5-ethynyl-2-({2-[4-(methanesulfonylmethyl)piperidin-1-yl]phenyl}amino)pyrido[2,3-d]pyrimidin-7-yl]urea;
1-(5-Ethynyl-2-{[4-(morpholin-4-yl]phenyl}aminopyrido[2,3-d]pyrimidin-7-yl)-1,3-diazaspiro[4.4]nonan-2-one;
1-[5-Ethynyl-2-({4-[4-(oxetan-3-yl)piperazin-1-yl]phenyl}amino)pyrido[2,3-d]pyrimidin-7-yl]-1,3-diazaspiro[4.4]nonan-2-one;
N-{4-[(5-Ethynyl-7-{2-oxo-1,3-diazaspiro[4.4]nonan-1-yl}pyrido[2,3-d]pyrimidin-2-yl)amino]phenyl}-N-methylacetamide;
2-(Dimethylamino)-N-{4-[(5-ethynyl-7-{2-oxo-1,3-diazaspiro[4.4]nonan-1-yl}pyrido[2,3-d]pyrimidin-2-yl)amino]phenyl}-N-methylacetamide;
1-[2-({4-[2-(Dimethylamino)ethoxy]phenyl}amino)-5-ethynyl)pyrido[2,3-d]pyrimidin-7-yl]-1,3-diazaspiro[4.4]nonan-2-one;
1-{5-Ethynyl-2-[(4-methanesulfonylphenyl)amino]pyrido[2,3-d]pyrimidin-7-yl}-1,3-diazaspiro[4.4]nonan-2-one;
4-[(5-Ethynyl-7-{2-oxo-1,3-diazaspiro[4.4]nonan-1-yl}pyrido[2,3-d]pyrimidin-2-yl)amino]-N, N-dimethylbenzenesulfonamide;
N-[2-(dimethylamino)ethyl]-4-[(5-ethynyl-7-{2-oxo-1,3-diazaspiro[4.4]nonan-1-yl}pyrido[2,3-d]pyrimidin-2-yl)amino]-N-methylbenzenesulfonamide;
(5S)-1-(5-Ethynyl-2-{[4-(4-methylpiperazin-1-yl)phenyl]amino}pyrido[2,3-d]pyrimidin-7-yl)-5-isopropylmidazolidin-2-one;
(5R)-1-(5-Ethynyl-2-{[4-(4-methylpiperazin-1-yl)phenyl]amino}pyrido[2,3-d]pyrimidin-7-yl)-5-isopropylmidazolidin-2-one;
(5S)-1-(5-Ethynyl-2-{[4-(4-methylpiperazin-1-yl)phenyl]amino}pyrido[2,3-d]pyrimidin-7-yl)-5-(methoxymethyl)imidazolidin-2-one;
3-(5-Ethynyl-2-{[4-(4-methylpiperazin-1-yl)phenyl]amino}pyrido[2,3-d]pyrimidin-7-yl)-4-isopropyl-1-methylmidazolidin-2-one;
1-(5-Ethynyl-2-{[4-(morpholin-4-yl)phenyl]amino}pyrido[2,3-d]pyrimidin-7-yl)-3-methyl-1,3-diazaspiro[4.4]nonan-2-one;
1-[5-Ethynyl-2-({4-[4-(oxetan-3-yl)piperazin-1-yl]phenyl}amino)pyrido[2,3-d]pyrimidin-7-yl]-3-methyl-1,3-diazaspiro[4.4]nonan-2-one;
N-{4-[(5-Ethynyl-7-{3-methyl-2-oxo-1,3-diazaspiro[4.4]nonan-1-yl}pyrido[2,3-d]pyrimidin-2-yl)amino]phenyl}-N-methylacetamide;
2-(Dimethylamino)-N-{4-[(5-ethynyl-7-{3-methyl-2-oxo-1,3-diazaspiro[4.4]nonan-1-yl}pyrido[2,3-d]pyrimidin-2-yl)amino]phenyl}-N-methylacetamide;
1-[2-({4-[2-(Dimethylamino) ethoxy]phenyl}amino)-5-ethynyl)pyrido[2,3-d]pyrimidin-7-yl]-3-methyl-1,3-diazaspiro[4.4]nonan-2-one;
1-{5-Ethynyl-2-[(4-methanesulfonylphenyl)amino]pyrido [2,3-d]pyrimidin-7-yl}-3-methyl-1,3-diazaspiro [4.4]nonan-2-one;
4-[(5-Ethynyl-7-{3-methyl-2-oxo-1,3-diazaspiro[4.4]nonan-1-yl}pyrido[2,3-d]pyrimidin-2-yl)amino]-N, N-dimethylbenzenesulfonamide;
3-Cyclopentyl-1-[5-ethynyl-2-((2-[3-(methanesulfonylmethyl)azetidin-1-yl]phenylamino)pyrido[2,3-d]pyrimidin-7-yl]urea;
4-Cyclopentyl-3-(5-ethynyl-2-{[4-(4-methylpiperazin-1-yl) phenyl]amino}pyrido[2,3-d]pyrimidin-7-yl)-1,3-oxazolidin-2-one;
4-(Cyclopentylmethyl)-3-(5-ethynyl-2-{[4-(4-methylpiperazin-1-yl)phenyl]amino}pyrido[2,3-d]pyrimidin-7-yl)-1,3-oxazolidin-2-one;
3-(5-Ethynyl-2-{[4-(4-methylpiperazin-1-yl)phenyl]amino}pyrido[2,3-d]pyrimidin-7-yl)-5-isopropyl-1,3-oxazolidin-2-one;
1-(5-Ethynyl-2-{[4-(4-methylpiperazin-1-yl)phenyl]amino}pyrido[2,3-d]pyrimidin-7-yl)-3-oxa-1-azaspiro[4.4]nonan-2-one;
4-Cyclopentyl-3-(5-ethynyl-2-{[4-(piperazin-1-yl)phenyl]amino}pyrido[2,3-d]pyrimidin-7-yl)-1,3-oxazolidin-2-one;
3-(5-Ethynyl-2-{[4-(4-methylpiperazin-1-yl)phenyl]amino}pyrido[2,3-d]pyrimidin-7-yl)-4-(methoxymethyl)-1,3-oxazolidin-2-one;
1-cyclopentyl-3-(5-ethynyl-2-((2-methylsoindolin-4-yl)amino)pyrido[2,3-d]pyrimidin-7-yl)urea;
1-cyclopentyl-3-(5-ethynyl-2-((2-(3-((methylsulfonyl)methyl)piperidin-1-yl)phenyl)amino)pyrido[2,3-d]pyrimidin-7-yl)urea;
1-cyclopentyl-3-(5-ethynyl-2-((3-(methyl((1s,3s)-3-((methylsulfonyl)methyl)cyclobutyl)amino)phenyl)amino)pyrido[2,3-d]pyrimidin-7-yl)urea;
1-cyclopentyl-3-(5-ethynyl-2-((3-(methyl((1s,4s)-4-((methylsulfonyl)methyl)cyclohexyl)amino)phenyl)amino)pyrido[2,3-d]pyrimidin-7-yl)urea;
1-cyclopentyl-3-(5-ethynyl-2-((3-(methyl((1r,4r)-4-((methylsulfonyl)methyl)cyclohexyl)amino)phenyl)amino)pyrido[2,3-d]pyrimidin-7-yl)urea;
1-cyclopentyl-3-(5-ethynyl-2-((3-(methyl((1r,3r)-3-((methylsulfonyl)methyl)cyclobutyl)amino)phenyl)amino)pyrido[2,3-d]pyrimidin-7-yl)urea;
1-cyclopentyl-3-(5-ethynyl-2-((4-(4-methylpiperazin-1-yl)phenyl)amino)pyrido[2,3-d]pyrimidin-7-yl)-1,3-dimethylurea; and
5-cyclopentyl-1-(5-ethynyl-2-((4-(4-methylpiperazin-1-yl)phenyl)amino)pyrido[2,3-d]pyrimidin-7-yl)imidazolidin-2-one.

22. A pharmaceutical composition comprising a compound according to any one of claims 1 to 21, or a pharmaceutically acceptable salt, hydrate or solvate thereof, and a pharmaceutically acceptable excipient.

23. A compound according to any one of claims 1 to 21, or a pharmaceutically acceptable salt thereof, or a pharmaceutical composition according to claim 22 for use in:
(i) therapy;
(ii) the treatment of a disease or condition in which EGFR activity is implicated;
(iii) the treatment of a disease or condition associated with aberrant activity of EGFR;
(iv) the treatment of proliferative disorders (e.g. cancer or benign neoplasms);
(v) the treatment of a cancer;
(vi) the treatment of an EGFR positive cancer, optionally selected from head and neck cancer, brain cancer, breast cancer, colon cancer and/or lung cancer;
(vii) EGFR positive non-small cell lung cancer;
(viii) the treatment of a cancer expressing a mutated form of EGFR (for example EGFR comprising a T790M mutation, a deletion in exon 19 (e.g. A740-A750), an exon 20 insertion, a mutation at L858R and/or a C797S mutation);
(ix) the treatment of a cancer resistant to treatment with a third generation EFGR inhibitor, e.g. osimertinib, lazertinib (YH25448), EGF816, olmutinib, PF-06747775, avitinib and/or rociletinib;
(x) the treatment of non-small cell lung cancer resistant to treatment with osimertinib.
